(19) United States
(12) Patent Application Publication
(10) Pub. No.: US 2011/0236897 A1
(43) Pub. Date: Sep. 29, 2011

(54) NOVEL MARKERS FOR DIAGNOSING BRAIN DISEASE CAUSED BY BRAIN INJURY AND USE THEREOF

(76) Inventors: Suk Woo NAM, Seoul (KR); Jung Woo Eun, Seoul (KR)

(21) Appl. No.: 12/877,084

(22) Filed: Sep. 7, 2010

(30) Foreign Application Priority Data
Mar. 26, 2010 (KR) 10-2010-27210

(51) Int. Cl. C12Q 1/68; C07H 21/04; C07K 14/00; C12N 9/50; C12N 9/16; C12N 9/12; C12N 9/90; G01N 33/53

(52) U.S. Cl. 435/6.12; 536/23.5; 530/350; 435/219; 435/194; 435/196; 435/233; 435/7.92; 436/501; 435/7.21; 435/6.16

(57) ABSTRACT

The present invention relates to novel markers for diagnosing brain disease caused by brain injury and the use thereof. More particularly, the present invention relates to novel diagnostic markers for brain disease caused by brain injury, which are identified by administering an MDMA drug, a diagnostic composition for brain disease caused by brain injury, a kit, a microarray, and a method for diagnosing brain disease caused by brain injury using the same, and relates to a method for screening a material for preventing or treating brain disease and a composition for preventing or treating brain disease caused by brain injury including the material. It was found that the expression amount of the marker genes of the present invention in tissues or cells of a patient with brain injury was over-expressed or under-expressed compared with that in normal tissues or cells. Thus, when used as diagnostic markers for brain disease caused by brain injury, the marker genes of the present invention can quickly and accurately diagnose and predict brain disease caused by brain injury at an early stage, and, particularly, can diagnose and predict brain disease caused by brain injury, which could be induced by an MDMA drug in the next generation.

### TECHNICAL FIELD

The present invention relates to novel diagnostic markers for brain disease caused by brain injury, which can effectively diagnose and predict brain disease caused by brain injury at an early stage, a diagnostic kit for brain disease caused by brain injury, a microarray, a diagnostic composition for brain disease caused by brain injury, and a method for diagnosing brain disease caused by brain injury using the diagnostic markers for brain disease caused by brain injury.

### BACKGROUND ART

“Brain damage” or “brain injury” (BI); means the destruction or degeneration of brain cells, often with an implication that the loss is significant in terms of functioning or conscious experience. It is a common and very broad in scope, such that in medicine a vast range of specific diagnoses exist. Brain injuries occur due to a wide range of internal and external factors. A common category with the greatest number of injuries is traumatic brain injury (TBI) following physical trauma or head injury from an outside source, and the term acquired brain injury (ABI) is used in appropriate circles, to differentiate brain injuries occurring after birth from injury due to a disorder or congenital malady.

Although the exact causes of brain damage are yet to be identified, possible causes of widespread (diffuse) brain damage include prolonged hypoxia (shortage of oxygen), poisoning by teratogens (including alcohol), infection, and neurological illness. Chemotherapy can cause brain damage to the neural stem cells and oligodendrocyte cells that produce myelin. Other common causes of brain damage are traumatic brain injury, stroke, aneurysm, surgery, other neurological disorder, and heavy metals poisoning including.

MDMA (3,4-methylenedioxymethamphetamine; ecstasy) has a ring-substituted phenyl-isopropylamine structure and is similar to amphetamines and hallucinogens in composition. MDMA is a psycho-stimulant in humans and causes hyperactivity and hyperthermia in rats and mice (Salzmann, J., Canestrelli, C., Noble, F., Marie-Claire, C., 2006, Analylsis of transcriptional responses in the mouse dorsal striatum following acute 3, 4-methylenedioxymethamphetamine (ecstasy): identification of extracelluar signal-regulated kinase-ontrolled genes. Neuroscience 137, 473-482).

According to the data reported so far, exposure to MDMA damages brain serotonin (5-hydroxytryptamine, 5-HT) and dopamine (DA) neurons, depending on the animal species and conditions of administration, including non-human primates (Green et al., 2003; Mithoefer et al., 2003). MDMA blocks the reuptake of 5-HT, reverses its flow, and induces the cytosolic release of 5-HT (Banasr et al., 2004; O'Hearn et al., 1988; Ricaurte et al., 2000; Shankaran et al., 1999; Thiriet et al., 2002). In addition, MDMA has been associated with neurotoxic effects on the dopaminergic system (Camarero et al., 2002; Itzhak et al., 2003; Mithoefer et al., 2003; Ricaurte et al., 2002; Xie et al., 2004; Yan et al., 2002).

Recently, it was reported that the recreational use of MDMA has increased among adolescents and college students. As the majority of MDMA users are young adults of reproductive age, studies on neurological changes due to the long-term use of MDA are being carried out. In addition, it has also been reported that prenatal exposure to MDMA may result in very severe brain damage (Schuster et al., 1998).

However, to explain the mechanisms that mediate prenatal modulation of MDMA responses is still difficult and no systematic approaches assessed the long-term responses to MDMA during the prenatal and developmental periods.

### DISCLOSURE

### Technical Problem

The present inventors have completed the present invention upon identifying novel markers for predicting or diagnosing brain disease caused by brain injury by using functional genomics methods which can provide mechanism-based assays for predicting toxin risks for the brain of a human.

Accordingly, it is an object of the present invention to provide novel markers for diagnosing brain disease caused by brain injury, which can predict or diagnose brain disease caused by brain injury.

It is another object of the present invention to provide a diagnostic composition for brain disease caused by brain injury, including a material for measuring the expression level of a gene or the amount of protein expressed from the gene that can be used as a diagnostic marker for brain disease caused by brain injury, and a diagnostic microarray for brain disease caused by brain injury.

It is still another object of the present invention to provide a method for predicting and diagnosing brain disease caused by brain injury, the method including the steps of: (a) measuring the expression levels of one or more genes selected from 90 genes listed in the following Table from a biological sample of a patient suspected of brain disease caused by brain injury or the amount of protein expressed therefrom; and (b) measuring the expression levels of genes from samples of a normal control group or the amount of protein expressed therefrom and comparing the same with a measurement result of step (a).

It is yet another object of the present invention to provide a method for screening a material for preventing or treating brain disease.

It is a further object of the present invention to provide a method for screening diagnostic markers for brain disease caused by brain injury.

### Technical Solution

To accomplish the aforementioned objects of the present invention, there are provided markers for diagnosing brain disease caused by brain injury, including one or more genes selected from 90 genes capable of diagnosing brain disease caused by brain injury or protein expressed therefrom.

In one embodiment of the present invention, the brain disease caused by brain injury may be induced by MDMA (3,4-methylenedioxymethamphetamine hydrochloride).

In one embodiment of the present invention, the brain disease caused by brain injury may be disease associated with serotonin or dopamine neurons.

In one embodiment of the present invention, the markers may predict or diagnose brain disease caused by brain injury, which is induced by MDMA (3,4-methylenedioxymethamphetamine hydrochloride) in the next generation.

Furthermore, the present invention provides a diagnostic composition for brain disease caused by brain injury, the composition including a material for measuring the expression levels of one or more genes selected from 90 genes identified in the present invention or the amount of protein expressed from the genes.

In one embodiment of the present invention, the material may be a primer for amplifying one of the 90 genes or an antibody against protein expressed from the gene.

Furthermore, the present invention provides a diagnostic kit for brain disease caused by brain injury, including the diagnostic composition for brain disease caused by brain injury of the present invention, and a diagnostic microarray for brain disease caused by brain injury, including the markers of the present invention.

Furthermore, the present invention provides a method for predicting and diagnosing brain disease caused by brain injury, the method including the steps of: (a) measuring the expression levels of one or more genes selected from 90 genes listed in the following Table from a biological sample of a patient suspected of brain disease caused by brain injury or the amount of protein expressed therefrom; and (b) measuring the expression levels of genes from samples of a normal control group or the amount of protein expressed therefrom and comparing the same with a measurement result of step (a).

In one embodiment of the present invention, the biological sample may be a cerebral cortical tissue or cell.

In one embodiment of the present invention, the assay may be selected from the group consisting of reverse transcriptase-polymerase chain reaction, real time-polymerase chain reaction, Western blotting, Northern blotting, ELISA (enzyme linked immunosorbent assay), RIA (radioimmunoassay), radioimmunodiffusion, and immunoprecipitation assay.

In one embodiment of the present invention, if the expression level of any one of the 1^{st} to 36^{th} genes among the 90 genes or the amount of protein expressed therefrom is under-expressed compared with samples of a normal control group, it can be concluded that brain disease caused by brain injury was induced.

In one embodiment of the present invention, if the expression level of any one of the 37^{st} to 90^{th} genes among the 90 genes or the amount of protein expressed therefrom is over-expressed compared with samples of a normal control group, it can be concluded that brain disease caused by brain injury was induced.

Furthermore, the present invention provides a method for screening a material for preventing or treating brain disease.

Furthermore, the present invention provides a method for screening diagnostic markers for brain disease caused by brain injury.

### Advantageous Effects

It was found that the expression amount of the marker genes of the present invention in tissues or cells of a patient with brain injury was under-expressed or over-expressed compared with that in normal tissues or cells. Thus, when used as diagnostic markers for brain disease caused by brain injury, the marker genes of the present invention can quickly and accurately diagnose and predict brain disease caused by brain injury at an early stage, and, particularly, can diagnose and predict brain disease caused by brain injury, which could be induced by an MDMA drug in the next generation.

### DESCRIPTION OF DRAWINGS

The above and other objects and features of the present invention will become apparent from the following description of the preferred embodiments given in conjunction with the accompanying drawings, in which:

FIG. 1 is a schematic view of an experimental design according to an embodiment of the present invention;

FIG. 2 shows differential expression profiles of mouse brain cerebral cortex after prenatal exposure of MDMA (20 mg/kg/day) according to one embodiment of the present invention, in which (A) shows the marked transcriptional changes in cerebral cortex in both male and female pups following the prenatal exposure to MDMA after single dose ingestion, both female (B) and male (C) offspring reflecting transcriptomic changes on their cerebral cortex;

FIG. 3 shows the identification of large-scale genetic elements changed by prenatal MDMA exposure on female and male offspring according to one embodiment of the present invention, in which (A) and (B) shows transcriptional changes on the cerebral cortex of the females and males, respectively; and

FIG. 4 shows the functional classification of differentially expressed genes after exposure to MDMA using KEGG pathway database, in which (A) shows changes in the females and (B) shows changes in the males.

### BEST MODE FOR THE INVENTION

The present invention is characterized in that: there is provided diagnostic markers for brain disease caused by brain injury, which can diagnose and predict brain disease caused by brain injury, and more particularly, the present invention is characterized in that: there is provided a marker for diagnosing brain disease caused by brain injury which is induced by exposure to an MDMA drug.

In general, in the case of adult exposure to MDMA, MDMA has been shown to cause acute and long-lasting neurotoxicity (Green et al., 2003). However, there is limited information on the intracellular transcriptional response to MDMA. Most prior studies have focused on the effects of MDMA in adult animals. However, because of the increase in use of MDMA among the young that are of childbearing age, it is important to determine whether MDMA has deleterious effects on the developing fetus and the next generation after in utero exposure.

Hereupon, the present inventors screened genes, whose expression patterns differ due to MDMA exposure, by using young mice as experimental animals in order to investigate the effects of MDMA during adolescence or prenatal life.

That is, according to one embodiment of the present invention, we assumed that the offspring of parents (F1 generation) exposed to MDMA might be indirectly affected by MDMA. Thus, to investigate whether this indirect exposure of MDMA to F1 (first generation of offspring), during the prenatal period of early fetal development, affected the transcriptomic configuration of the cerebral cortex of the brain in the F1 generation, we analyzed the differential gene expression of the F1 at 11-weeks after delivery, and compared the results with a control group not exposed to MDMA.

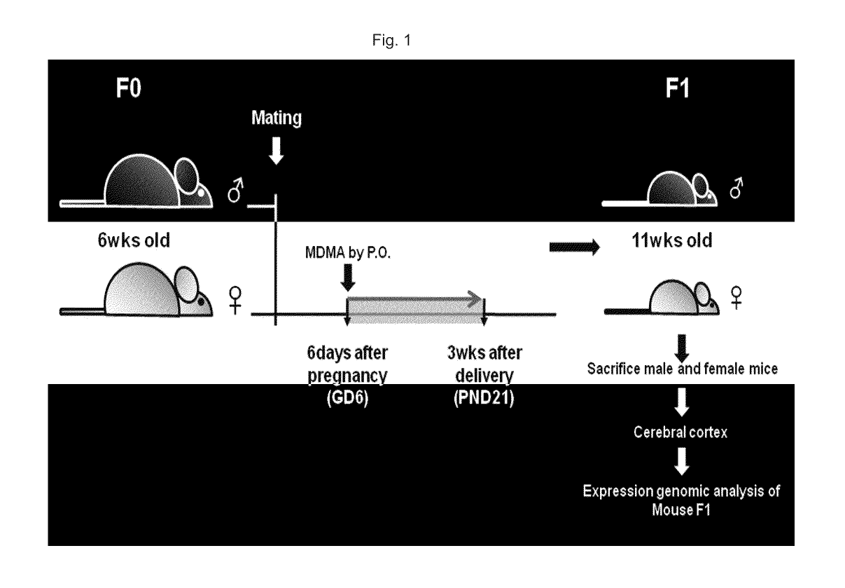

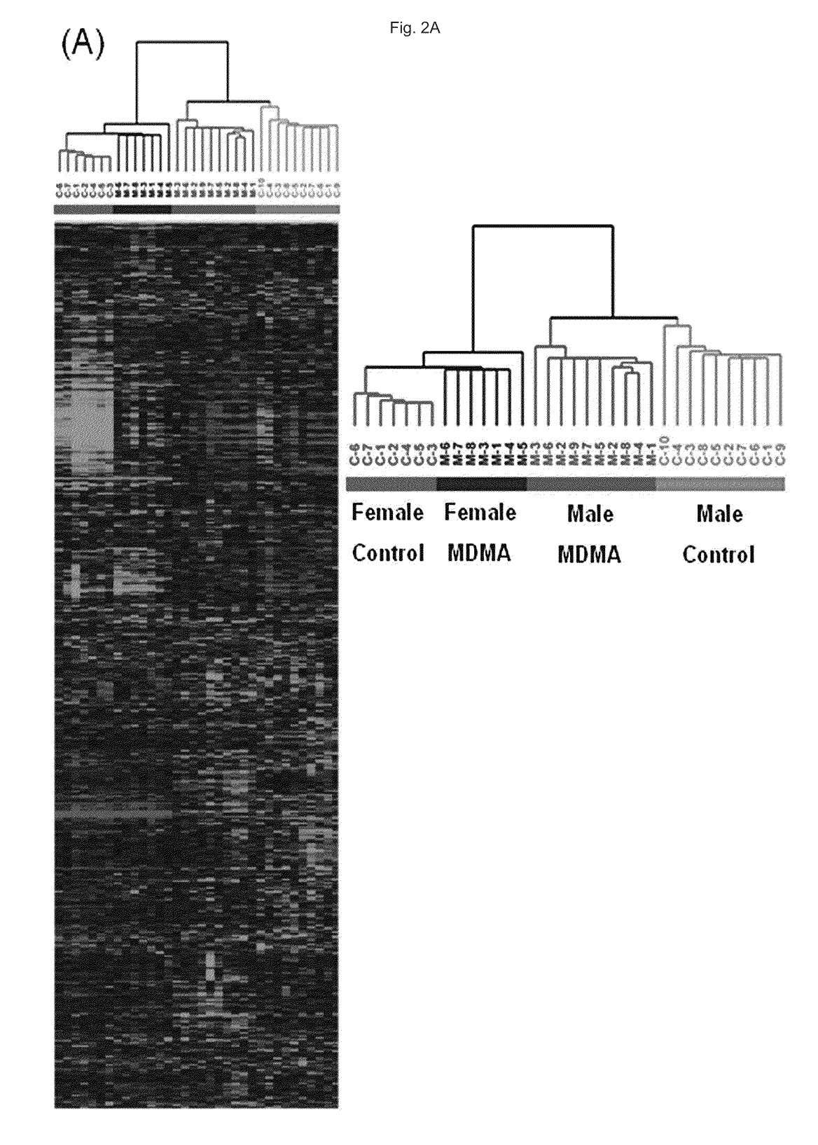

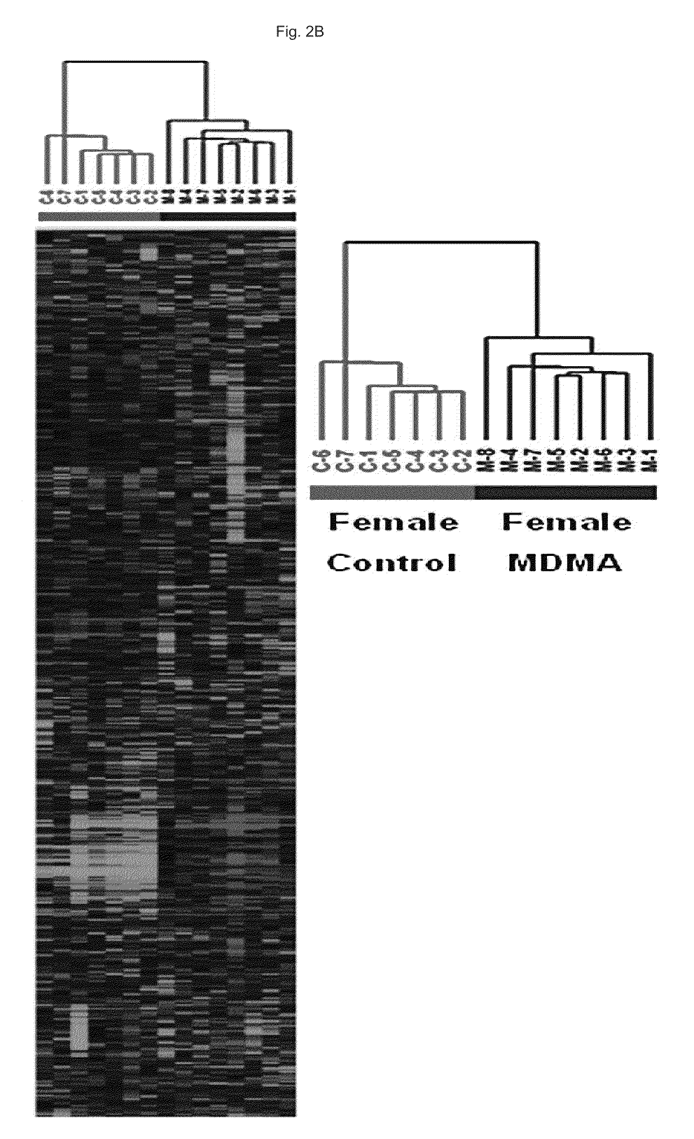

The results showed that the transcriptomic configuration of the prenatally exposed F1 generation had a different molecular signature compared to the control group; these comprehensive molecular changes associated with the prenatal exposed F1 generation are shown in the dendrogram (see FIGS. 1 and 2).

The results showed that prenatal exposure to MDMA elicited transcriptomic configuration changes in the mouse cerebral cortex in the F1 generation. There were 1,784 and 804 genetic elements identified as MDMA associated molecular signatures in both F1 females and males. The altered epigenetic regulation appeared to be permanent during development after continuous exposure to MDMA.

As a result, the present inventors have found that the genes showing changes in expression can be used as markers for diagnosing brain disease caused by brain injury.

Further, the present inventors selected 90 common genes from 1,784 genes of females and 804 genes of males identified in one embodiment of the present invention, and presented a list of the genes in the following Table. In the following Table, the 1^{st} to 36^{th} genes are genes whose expression was reduced following MDMA exposure, and the 37^{st} to 90^{th} genes are genes whose expression was over-expressed following MDMA exposure. The base sequences of the 12 genes, whose names are not defined, among the genes of the table, are listed in the sequence list accompanying this specification.

| | | | GenBank | Expression |
|---|---|---|---|---|
| No. | Probe ID | Gene Name | accession # | change |
| 1 | 299424 | Unknown (Sequence No. 1) | AK033066.1 | down- |
| 2 | 305559 | Unknown (Sequence No. 2) | | regulation |
| 3 | 328759 | fibroblast growth factor 20 | NM_030610.1 | (under- |
| 4 | 354854 | RIKEN cDNA 4930451A11 gene | AK015436.1 | expression |
| 5 | 355052 | Unknown (Sequence No. 3) | AK037760.1 | compared |
| 6 | 355535 | cytotoxic T lymphocyte-associated | AY034578.1 | with |
| | | protein 2 beta | | control |
| 7 | 361748 | T lymphoma oncogene | NM_011601.1 | group) |
| 8 | 368174 | RIKEN cDNA 2010323F13 gene | NM_177157.2 | |
| 9 | 392437 | RIKEN cDNA 4930566F21 gene | AK016234.1 | |
| 10 | 401554 | Ribosomal protein S2 | AK004568.1 | |
| 11 | 424366 | unknown(Sequence No. 4) | | |
| 12 | 428440 | otogelin | NM_013624.1 | |
| 13 | 432053 | RIKEN cDNA 4933407A17 gene | AK016710.1 | |
| 14 | 433675 | similar to T-cell receptor alpha chain V | M23096.1 | |
| | | region PHDS58 precursor | | |
| 15 | 460185 | predicted gene 3364 | AK090251.1 | |
| 16 | 478590 | ring finger protein 149 | AK040602.1 | |
| 17 | 497466 | cyclic nucleotide gated channel alpha 2 | NM_007724.2 | |
| 18 | 513268 | RIKEN cDNA C030016D13 gene | NM_175354.2 | |
| 19 | 515525 | unknown(Sequence No. 5) | AK053269.1 | |
| 20 | 520252 | unknown(Sequence No. 6) | | |
| 21 | 524988 | early growth response 1 | NM_007913.2 | |
| 22 | 525785 | hypothetical protein 9130430E04 | NM_183203.1 | |
| 23 | 530939 | RIKENcDNA9330176C04gene; | NM_175420.1 | |
| | | solutecarrierfamily22 | | |
| | | (organiccationtransporter), member13 | | |
| 24 | 566320 | unknown(Sequence No. 7) | | |
| 25 | 615742 | olfactory receptor 1350 | NM_146389.1 | |
| 26 | 749804 | RIKEN cDNA 1200002N14 gene | NM_027878.1 | |
| 27 | 762310 | RIKEN cDNA 4833417J20 gene | AK014716.1 | |
| 28 | 781797 | olfactory receptor 704 | AY317825.1_{—} | |
| | | | CDS_1 | |
| 29 | 783471 | unknown(Sequence No. 8) | AK037520.1 | |
| 30 | 824655 | unknown(Sequence No. 9) | AK037572.1 | |
| 31 | 838496 | RIKEN cDNA A430057L12 gene | AK078093.1 | |
| 32 | 841082 | RIKEN cDNA 9330198N18 gene | AK020395.1 | |
| 33 | 847317 | myogenic differentiation 1 | NM_010866.1 | |
| 34 | 850104 | gene model 44 | XM_907821 | |
| 35 | 910205 | RIKEN cDNA F630021I08 gene | NM_178743.2 | |
| 36 | 932668 | early growth response 2 | NM_010118.1 | |
| 37 | 298812 | RIKEN cDNA 1700007H16 gene | NM_027945.1 | up- |
| 38 | 334928 | matrix metalloproteinase 15 | NM_008609.2 | regulation |
| 39 | 338997 | Ras association (RalGDS/AF-6) domain | NM_175445.3 | (over- |
| | | family 2 | | expression |
| 40 | 339413 | cDNA sequence BC020354 | NM_198110.1 | compared |
| 41 | 357505 | high density lipoprotein (HDL) binding | NM_133808.2 | with |
| | | protein | | control |
| 42 | 357970 | cyclin-dependent kinase 19 | AK019224.1 | group) |
| 43 | 370504 | potassiumvoltagegatedchannel, | NM_008420.3 | |
| | | Shab-relatedsubfamily, member1 | | |
| 44 | 391067 | kelch repeat and BTB (POZ) domain | NM_001164493.1 | |
| | | containing 9 | | |
| 45 | 396150 | transmembrane protein 120B | BC022593.1 | |
| 46 | 398003 | phosphofructokinase, muscle | NM_021514.2 | |
| 47 | 444821 | unknown(Sequence No. 10) | | |
| 48 | 463085 | breast carcinoma amplified sequence 3 | NM_138681.2 | |
| 49 | 466313 | POU domain, class 3, transcription | NM_011141.1 | |
| | | factor 1 | | |
| 50 | 471089 | RIKEN cDNA B930093C12 gene | NM_172945.1 | |
| 51 | 472358 | sterile alpha motif domain containing | BC049740.1 | |
| | | 4 | | |
| 52 | 475067 | Rho guanine nucleotide exchange factor | NM_027144.1 | |
| | | (GEF) 12 | | |
| 53 | 488058 | cDNA sequence BC052066 | NM_172761.1 | |
| 54 | 491436 | fuzzy homolog (Drosophila) | BC045601.1 | |
| 55 | 498825 | SEC5-like 1 (S. cerevisiae) | NM_025588.2 | |
| 56 | 501289 | presenilin 1 | NM_008943.1 | |
| 57 | 526436 | insulin-like growth factor binding | NM_010517.2 | |
| | | protein 4 | | |
| 58 | 531607 | Unknown (Sequence No. 11) | | |
| 59 | 540378 | RNA binding motif protein 8 | NM_025875.1 | |
| 60 | 544433 | RIKEN cDNA A830097P10 gene | NM_173780.2 | |
| 61 | 555234 | protein phosphatase 5, catalytic subunit | NM_011155.1 | |
| 62 | 555859 | BMS1-like, ribosome assembly protein | NM_194339.1 | |
| | | (yeast) | | |
| 63 | 559958 | gene rich cluster, A gene | NM_013533.2 | |
| 64 | 563270 | unknown(Sequence No. 12) | AK043558.1 | |
| 65 | 563705 | RIKEN cDNA 2210009G21 gene | NM_028834.1 | |
| 66 | 574782 | zinc finger, DHHC domain containing 14 | NM_146073.2 | |
| 67 | 597728 | RIKEN cDNA 6430548M08 gene | NM_172286.2 | |
| 68 | 602012 | dishevelled associated activator of | AY426536.1 | |
| | | morphogenesis 2 | | |
| 69 | 606287 | 3-monooxgenase/tryptophan 5-monooxgenase | NM_018871.2 | |
| | | activation protein, gamma polypeptide | | |
| 70 | 607746 | RIKEN cDNA 9330161C17 gene | NM_172614.2 | |
| 71 | 638437 | histocompatibility2, Tregionlocus9; | NM_010399.2 | |
| | | histocompatibility2, | | |
| | | Tregionlocus22; histocompatibility2, | | |
| | | Tregionlocus17; histocompatibility2, | | |
| | | Tregionlocus10 | | |
| 72 | 658280 | double C2, alpha | NM_010069.1 | |
| 73 | 668016 | RIKEN cDNA D930014A20 gene | NM_178811.3 | |
| 74 | 669220 | zinc finger, FYVE domain containing 20 | NM_030081.2 | |
| 75 | 675690 | RIKEN cDNA 2310075C17 gene | AK010164.1 | |
| 76 | 703524 | synaptotagmin 12 | NM_134164.2 | |
| 77 | 734020 | myosin XVIIIa | NM_011586.1 | |
| 78 | 734899 | wingless-related MMTV integration site | NM_009524.2 | |
| | | 5A | | |
| 79 | 746773 | RIKEN cDNA 2610511O17 gene | NM_152817.2 | |
| 80 | 757556 | XPA binding protein 2 | NM_026156.1 | |
| 81 | 769898 | UDP-N-acetyl-alpha-D-galactosamine: | NM_139272.2 | |
| | | polypeptideN- | | |
| | | acetylgalactosaminyltransferase2 | | |
| 82 | 829347 | RAD52 homolog B (S. cerevisiae) | NM_025654.1 | |
| 83 | 849739 | peptidylprolyl isomerase (cyclophilin)- | NM_026845.1 | |
| | | like 1 | | |
| 84 | 858428 | toll interacting protein | NM_023764.2 | |
| 85 | 894752 | ets variant gene 5 | NM_023794.2 | |
| 86 | 896014 | eukaryotictranslationinitiationfactor2, | NM_012010.2 | |
| | | subunit3, structuralgeneX-linked | | |
| 87 | 898183 | glucose phosphate isomerase 1 | NM_008155 | |
| 88 | 917144 | sodium channel, voltage-gated, type I, | NM_011322.2 | |
| | | beta polypeptide | | |
| 89 | 923175 | TRIO and F-actin binding protein | NM_138579.2 | |
| 90 | 925691 | mitochondrial solute carrier protein | NM_030054.2 | |

There is evidence to support gender differences in the psychological and physiological responses to MDMA. For example, female rats exposed to MDMA have significantly stronger locomotor activation than males, and in human studies, women have been reported to have more significant depression scores, perceptual changes, thought disturbances, and fear of loss of body control compared to men (Liechti et al., 2001; Verheyden et al., 2002; Walker et al., 2007). However, the data on gender differences is limited with regard to the psycho-stimulant effects, 5-HT function and subjective effects.

On the other hand, according to the results of the present invention, 1,784 genes in the F1 females and 804 genes in the F1 males, after prenatal MDMA exposure, were identified to be associated with molecular signatures. Thus, the transcriptomic configurations of the male and female F1 brain after prenatal exposure to MDMA may provide additional information on gender differences in response to stimulants.

Accordingly, the present invention can provide markers for diagnosing brain disease caused by brain injury, including one or more genes selected from 90 genes capable of diagnosing brain disease caused by brain injury or protein expressed therefrom.

In the context of the present invention, the term “diagnosis”, as used herein, refers to evaluation of the presence or properties of pathological states. With respect to the objects of the present invention, the diagnosis is to determine the incidence of brain disease caused by brain injury by determining the expression or lack of expression of diagnostic markers for brain disease caused by brain injury. In addition, in the context of the present invention, the “diagnosis” includes determining the onset, progression, or amelioration of brain disease caused by brain injury by determining the expression or lack of expression of diagnostic markers for brain disease caused by brain injury and the expression levels thereof.

The terms “markers for diagnosing”, “markers for diagnosis”, or “diagnostic markers”, as used herein, are intended to indicate substances that can diagnose brain disease by distinguishing diseased brain cells from normal cells, and includes organic biological molecules, quantities of which increase or decrease in diseased brain cells compared to normal cells, such as polypeptides or nucleic acids (e.g., mRNA, etc.), lipids, glycolipids, glycoproteins, and sugars (monosaccharides, disaccharides, oligosaccharides, etc.). The diagnostic markers for brain disease caused by brain injury provided in the present invention may be the aforementioned 90 genes, whose expression is over-expressed or under-expressed compared with normal cells, or protein expressed from the genes.

In the present invention, the “brain disease caused by brain injury” includes all types of diseases that occur in the short term or in the long term due to all types of brain injury causing the destruction or degeneration of brain cells. Although not limited to specific types of diseases, the “brain disease caused by brain injury” may be preferably disease induced by MDMA (3,4-methylenedioxymethamphetamine hydrochloride). Further, in the present invention, the brain disease caused by brain injury may be preferably disease associated with serotonin or dopamine neurons.

Further, in the present invention, the markers may predict or diagnose brain disease caused by brain injury, which is induced by MDMA (3,4-methylenedioxymethamphetamine hydrochloride) in the next generation.

The present invention provides a diagnostic composition for brain disease caused by brain injury, the composition including a material for measuring the expression levels of the marker genes of the present invention or the amount of protein expressed from the genes.

Further, the present invention provides a method for screening diagnostic markers for brain disease caused by brain injury according to the present invention, preferably, a method for screening diagnostic markers for predicting or diagnosing brain disease caused by brain injury, which is induced by MDMA (3,4-methylenedioxymethamphetamine hydrochloride).

The method for screening diagnostic markers for brain disease caused by brain injury according to the present invention may include the step of analyzing the expression profiles of genes expressed from a sample administered with MDMA and a sample not administered with MDMA, and analyzing and identifying genes showing differences in the level of gene expression in a group administered with MDMA, compared to a group not administered with MDMA.

In one embodiment of the present invention, total RNA was isolated from tissue administered with MDMA and tissue not administered with MDMA, and then genes whose expressions are over-expressed or under-expressed in a group administered with MDMA, compared with a group not administered with MDMA, were screened by expression profiling analysis of DNA microarray data.

In the present invention, the expression levels of the genes preferably refer to the level of mRNA, i.e., the amount of mRNA, at which the genes are expressed. A material for measuring the level may include a primer or probe specific to the genes. In the present invention, the primer or probe specific to the genes may be a primer or probe capable of specifically amplifying the entire or specific regions of each of the 90 genes, and the primer or probe can be designed by methods known in the art.

In the present invention, the “primer” used herein is a single-stranded oligonucleotide functioning as an origin of polymerization of template DNA under an appropriate conditions (i.e., 4 types of different nucleoside triphosphates and polymerases) at a suitable temperature and in a suitable buffer solution. The length of the primer may vary according to various factors, for example, temperature and the use of the primer. The sequence of the primer is not required to be perfectly complementary to a part of the sequence of the template. The primer may have sufficient complementarity to be hybridized with the template and perform intrinsic functions of the primer. Thus, the primer of the present invention is not required to be perfectly complementary to the sequence of the nucleotide that is a template. The primer of the present invention may have sufficient complementarity to be hybridized with the sequence and perform intrinsic functions of the primer. Moreover, the primer of the present invention is preferably used for gene amplification.

The “amplification” refers to nucleic acid amplification. Examples of amplification techniques of genes are known in the art, and may include polymerase chain reaction (PCR), reverse-transcription polymerase chain reaction (RT-PCR), ligase chain reaction (LCR), transcription-mediated amplification (TMA), nucleic acid sequence-based amplification (NASBA), etc.

The term “probe” as used herein refers to a linear oligomer of natural or modified monomers or linkages, including deoxyribonucleotides, ribonucleotides and the like, capable of specifically hybridizing with a target nucleotide sequence, whether occurring naturally or produced synthetically. The probe of the present invention may be single stranded, preferably, an oligodeoxyribonucleotide. The probe of this invention can be comprised of naturally occurring dNMP (i.e., dAMP, dGMP, dCMP and dTMP), nucleotide analogs, or nucleotide derivatives. The probe of this invention can also include ribonucleotides. For example, the probe of this invention may include nucleotides with backbone modifications such as peptide nucleic acid (PNA) (M. Egholm et al., Nature, 365:566-568 (1993)), phosphorothioate DNA, phosphorodithioate DNA, phosphoramidate DNA, amide-linked DNA, MMI-linked DNA, 2′-O-methyl RNA, alpha-DNA and methylphosphonate DNA, nucleotides with sugar modifications such as 2′-O-methyl RNA, 2′-fluoro RNA, 2′-amino RNA, 2′-O-alkyl DNA, 2′-O-allyl DNA, 2′-O-alkynyl DNA, hexose DNA, pyranosyl RNA, and anhydrohexitol DNA, and nucleotides having base modifications such as C-5 substituted pyrimidines (substituents including fluoro-, bromo-, chloro-, iodo-, methyl-, ethyl-, vinyl-, formyl-, ethynyl-, propynyl-, alkynyl-, thiazolyl-, imidazolyl-, pyridyl-), 7-deazapurines with C-7 substituents (substituents including fluoro-, bromo-, chloro-, iodo-, methyl-, ethyl-, vinyl-, formyl-, alkynyl-, alkenyl-, thiazolyl-, imidazolyl-, pyridyl-), inosine, and diaminopurine.

In the present invention, materials for measuring protein levels may include “antibodies” including polyclonal antibodies, monoclonal antibodies, and recombinant antibodies that may specifically bind to proteins expressed from the 90 marker genes of the present invention.

The “antibodies” may be produced using techniques widely known in the art. Polyclonal antibodies may be produced using a method widely known in the art, which includes injecting the protein antigen into an animal and collecting blood samples from the animal to obtain sera containing antibodies. Such polyclonal antibodies may be prepared from a certain animal host, such as goats, rabbits, sheep, monkeys, horses, pigs, cows and dogs. Monoclonal antibodies may be prepared by a method widely known in the art, such as a hybridoma method (see, Kohler and Milstein (1976) European Journal of Immunology 6:511-519), or a phage antibody library technique (Clackson et al., Nature, 352:624-628, 1991; Marks et al., J. MoI. Biol., 222:58, 1-597, 1991). In addition, the antibodies of the present invention include complete forms, each of which consist of two full-length light chains and two full-length heavy chains, as well as functional fragments of antibody molecules. The functional fragments of antibody molecules refer to fragments retaining at least an antigen-binding function, and include Fab, F(ab′), F(ab′)2 and Fv.

Further, the present invention provides a diagnostic kit for brain disease caused by brain injury, including the diagnostic markers for brain disease caused by brain injury or the diagnostic composition for brain disease caused by brain injury, and the kit may include a material for measuring the expression levels of one or more genes selected from the 90 genes or the amount of protein expressed from the genes according to the present invention.

The material for measuring the expression levels of the genes or the amount of protein expressed from the genes may include primers, probes, or antibodies that are specific to protein, the definitions of which were described previously.

If the diagnostic kit for brain disease caused by brain injury of the present invention is used in the PCR amplification procedure, the kit of the present invention may selectively include reagents required for PCR amplification, for example buffer, DNA polymerase (for example, thermostable DNA polymerase obtained from Thermus aquaticus (Taq), Thermus thermophilus (Tth), Thermus filiformis, Thermis flavus, Thermococcus literalis or Pyrococcus furiosus (Pfu)), DNA co-polymerase and dNTPs. The kit of the present invention may be made of a plurality of packagings or compartments including the above reagent components.

Further, the present invention provides a diagnostic microarray for brain disease caused by brain injury, the microarray including the diagnostic markers for brain disease caused by brain injury.

In the microarray of the present invention, the primers, probes, or antibodies serve as hybridizable array elements and are immobilized on substrates. A preferable substrate includes suitable solid or semi-solid supporters, such as membrane, filter, chip, slide, wafer, fiber, magnetic or nonmagnetic bead, gel, tubing, plate, polymer, microparticle and capillary tube. The hybridizable array elements are arranged and immobilized on the substrate. Such immobilization occurs through chemical binding or covalent binding such as UV. In an example, the hybridizable array elements are bound to a glass surface modified to contain epoxy compound or aldehyde group or to a polylysine-coated surface by UV irradiation. Further, the hybridizable array elements are bound to a substrate through linkers (e.g. ethylene glycol oligomer and diamine).

If samples to be applied to the microarray of this invention are nucleic acids, they may be labeled, and hybridized with array elements on microarray. Various hybridization conditions are applicable, and for the detection and analysis of the extent of hybridization, various methods are available depending on labels used.

Further, the present invention provides a method for predicting and diagnosing brain disease caused by brain injury by measuring the expression levels of the marker genes or the level of protein expressed therefrom, the method including the steps of: (a) measuring the expression levels of one or more genes selected from 90 genes listed in the following Table from a biological sample of a patient suspected of brain disease caused by brain injury or the amount of protein expressed therefrom; and (b) measuring the expression levels of genes from samples of a normal control group or the amount of protein expressed therefrom and comparing the same with a measurement result of step (a).

The aforementioned method for measuring the expression levels of the marker genes or the level of protein may be carried out by including a known process for isolating mRNA or protein from a biological sample by using the known art.

In the present invention, the term “biological sample”, as used herein, refers to samples derived from living organisms which show a difference in the expression levels of the genes or in the level of protein from a normal control group depending on the degree of the onset or progression of brain disease caused by brain injury, and the samples may include tissues, cells, whole blood, serum, plasma, saliva, or urine, but are not limited thereto.

An assay of expression levels of the genes involves measuring the level of mRNA. Methods for measuring the level of mRNA include reverse transcriptase-polymerase chain reaction (RT-PCR), real time reverse transcriptase-polymerase chain reaction, RNase protection analysis, Northern blotting, DNA chip, etc, but are not limited thereto.

The measurement of protein levels can be carried out using an antibody. In this case, a marker protein in a biological sample and an antibody specific thereto form complexes, i.e., antigen-antibody complexes. The amount of formed antigen-antibody complexes may be quantitatively determined by measuring the signal size of a detection label. Such a detection label may be selected from the group consisting of enzymes, fluorescent substances, ligands, luminescent substances, microparticles, redox molecules and radioactive isotopes, but the present invention is not limited to the examples. Analysis methods for measuring protein levels include, but are not limited to, Western blotting, ELISA, radioimmunoassay (RIA), radioimmunodiffusion, ouchterlony immunodiffusion, rocket Immunoelectrophoresis, immunohistostaining, immunoprecipitation assay, complement fixation assay, FACS, and protein chip assay.

Accordingly, by the detection methods of the present invention, the mRNA expression level or protein level of a control group and the mRNA expression level or protein level of a patient with brain disease caused by brain injury or a patient suspected of having brain disease caused by brain injury can be determined, and the onset, progression, or prognosis of brain disease caused by brain injury can be predicted and diagnosed by comparing the expression level with that of the control group.

Moreover, in the method for predicting and diagnosing brain disease caused by brain injury according to the present invention, if the expression level of any one of the 1^{st} to 36^{th} genes among the 90 genes, which are brain damage marker genes, or the amount of protein expressed therefrom is under-expressed compared with samples of a normal control group, it is concluded that brain disease caused by brain injury was induced. On the other hand, if the expression level of any one of the 37^{st} to 90^{th} genes among the 90 genes or the amount of protein expressed therefrom is over-expressed compared with samples of a normal control group, it is concluded that brain disease caused by brain injury was induced.

Furthermore, the present invention provides a method for screening a material for preventing or treating brain disease, the method including the steps of: (a) contacting a sample to be analyzed with cerebral cortical cells of an individual with brain injury containing one or more genes selected from the group consisting of the 1^{st} to 36^{th} genes among the 90 marker genes for diagnosing brain disease caused by brain injury according to the present invention or protein expressed therefrom; (b) measuring the expression level of the selected gene or the amount or activity of protein expressed therefrom; and (c) as a measurement result of step (b), if the expression level of the selected gene or the amount or activity of protein expressed therefrom is over-expressed, determining the sample as the material for preventing or treating brain disease caused by brain injury.

In addition, the present invention provides a method for screening a material for preventing or treating brain disease, the method including the steps of: (a) contacting a sample to be analyzed with cerebral cortical cells of an individual with brain injury containing one or more genes selected from the group consisting of the 37^{st} to 90^{th} genes among the 90 marker genes for diagnosing brain disease caused by brain injury according to the present invention or protein expressed therefrom; (b) measuring the expression level of the selected gene or the amount or activity of protein expressed therefrom; and (c) as a measurement result of step (b), if the expression level of the selected gene or the amount or activity of protein expressed therefrom is under-expressed, determining the sample as the material for preventing or treating brain disease caused by brain injury.

According to the screening method of the present invention, damaged brain cells containing the gene or protein are first contacted to a sample to be analyzed. The term “sample” refers to a material used in the screening method for analyzing the influence on the expression level of the gene, the amount of protein, or the activity of protein. The sample includes chemical substances, nucleotides, antisense-RNA, siRNA (small interference RNA) and extract of natural source, but not limited to. Afterwards, the expression level of the gene, the amount of protein, or the activity of protein in the cells treated with the sample is measured. As a result of the measurement, if the expression level of the gene, the amount of protein or the activity of protein is measured to be over-expressed or under-expressed, the sample is determined to be a material for treating or preventing brain disease caused by brain injury.

The measurement of the expression level of the gene could be carried out by a variety of methods known in the art. For example, these methods can be carried out by using reverse transcriptase-polymerase chain reaction, real time-polymerase chain reaction, Western blotting, Northern blotting, ELISA (enzyme linked immunosorbent assay) RIA (radioimmunoassay), radioimmunodiffusion, and immunopreciptation assay, but are not limited thereto.

Further, the present invention provides a pharmaceutical composition for preventing or treating brain disease caused by brain injury, which includes, as an active ingredient, a material for over-expressing one or more genes selected from the group consisting of the 1^{st} to 36^{th} genes or the amount or activity of protein expressed therefrom or a material for under-expressing one or more genes selected from the group consisting of the 37st to 90th genes or the amount or activity of protein expressed therefrom.

The pharmaceutical composition of this invention may include, as an active ingredient, chemical substances, nucleotides, antisense oligonucleotides, siRNA oligonucleotides or extract of natural source. Preferably, the pharmaceutical composition of this invention may include, as an active ingredient, antisense or siRNA (small interference RNA) oligonucleotide having a complementary sequence to the nucleotide sequence of the marker genes of this invention.

In the present invention, the term “antisense nucleic acids”, as used herein, refers to DNA, RNA or derivatives thereof containing a nucleic acid sequence complementary to a specific mRNA sequence, and bind to a complementary sequence in mRNA and inhibit translation of mRNA into a protein. The antisense sequences of the present invention mean DNA or RNA sequences that are complementary to mRNA of the gene and are able to bind mRNA, and may inhibit translation, cytoplasmic translocation or maturation of mRNA or all other activities essential for overall biological functions.

The antisense nucleic acids may be modified at one or more positions of bases, sugars or backbones in order to have improved effectiveness (De Mesmaeker et al., Curr Opin Struct Biol., 5(3):343-55, 1995). The nucleic acid backbone may be modified, for example, with phosphorothioates, phosphotriesters, methyl phosphonates, short chain alkyl or cycloalkyl intersugar linkages, or short chain heteroatomic or heterocyclic intersugar linkages. Also, the antisense nucleic acids may contain one or more substituted sugar moieties. The antisense nucleic acids may also contain modified bases. Examples of the modified bases include hypoxanthine, 6-methyladenine, 5-methyl-pyrimidines (especially 5-methylcytosine), 5-hydroxymethylcytosine (HMC), glycosyl HMC, gentiobiosyl HMC, 2-aminoadenine, 2-thiouracil, 2-thiothymine, 5-bromouracil, 5-hyroxymethyluracil, 8-azaguanine, 7-deazaguanine, N6(6-aminohexyl) adenine, and 2,6-diaminopurine. In addition, the antisense nucleic acids of the present invention may be chemically bonded to one or more moieties or conjugates enhancing the activity and cellular uptake of the antisense nucleic acids. For example, liphophilic moieties include, but are not limited to, a cholesterol moiety, a cholesteryl moiety, cholic acid, a thioether, a thiocholesterol, an aliphatic chain, a phospholipid, a polyamine, a polyethylene glycol chain, adamantane acetic acid, a palmityl moiety, an octadecylamine moiety, and a hexylamino-carbonyl-oxycholesterol moiety. A method of preparing oligonucleotides comprising lipid moieties is well known in the art (U.S. Pat. Nos. 5,138,045, 5,218,105 and 5,459,255). The modified nucleic acids may have enhanced stability in the presence of nucleases and enhanced binding affinity to target mRNA.

Antisense oligonucleotides may be synthesized in vitro by an ordinary method and administered to the body, or may be synthesized in vivo. A method for synthesizing antisense oligonucleotides in vitro employs RNA polymerase I. A method for synthesizing antisense RNA in vivo involves performing transcription of antisense RNA using a vector containing a multicloning site (MCS) in the opposite direction. Such antisense RNA preferably contains a translation stop codon in its sequence to block translation into a peptide sequence.

The term “siRNA” used herein refers to a nucleic acid molecule mediating RNA interference or gene silencing (see WO 00/44895, WO 01/36646, WO 99/32619, WO 01/29058, WO 99/07409 and WO 00/44914). The siRNA to inhibit expression of a target gene provides effective gene knock-down method or gene therapy method.

The siRNA molecule of this invention may consist of a sense strand (having sequence corresponding to the mRNA sequence of the marker genes) and an antisense strand (having sequence complementary to the mRNA sequences), which are opposite to each other, and form a duplex structure. Alternatively, the siRNA molecule of this invention may have a single strand structure comprising self-complementary sense and antisense strands.

The siRNA of this invention is not restricted to a RNA duplex of which two strands are completely paired and may comprise non-paired portion such as mismatched portion with non-complementary bases and bulge with no opposite bases. The siRNA may comprise either blunt or cohesive end so long as it enables to silent the MLN 51 expression due to RNAi effect. The cohesive end may be prepared in 3′-end overhanging structure or 5′-end overhanging structure.

The siRNA may be constructed by inserting a short nucleotide sequence between self-complementary sense and antisense strands. The siRNA expressed forms a hairpin structure by intramolecular hybridization, resulting in the formation of stem-and-loop structure. The stem-and-loop structure is processed in vitro or in vivo to generate active siRNA molecule mediating RNAi.

The siRNA may be prepared by a method in which siRNA is directly synthesized in vitro and introduced into cells by transfection, or another method in which a siRNA expression vector or PCR-induced siRNA expression cassette, which is constructed to express siRNA in cells, is transformed or transfected into cells.

Moreover, the composition of this invention including gene-specific siRNA may include an agent for promoting the introduction of siRNA into a cell. The agent for promoting the intracellular introduction of siRNA may be generally a promoter for the introduction of nucleic acids. For example, liposomes may be used alone or in combination with a lipophilic carrier selected from among sterols including cholesterol, cholate and deoxycholic acid. For the intracellular introduction of siRNA, cationic polymers, such as poly-L-lysine, spermine, polysilazane, PEI (polyethylenimine), polydihydroimidazolenium, polyallylamine, chitosan, etc. or anionic polymers, such as succinylated PLL, succinylated PEI, polyglutamic acid, polyaspartic acid, polyacrylic acid, polymethacrylic acid, dextran sulfate, heparin, hyaluronic acid, etc., may be used.

Further, when an antibody specific to the protein is used as a material for over-expressing or under-expressing the expression and activity of the marker protein, the antibody can be coupled (e.g., covalently bonded) with pre-existing therapeutic agents directly or indirectly, that is, via a linker. Examples of the therapeutic agents coupled with antibodies include, but not limited to, radionucleotides, such as 131I, 90Y, 105Rh, 47Sc, 67Cu, 212Bi, 211At, 67Ga, 125I, 186Re, 188Re, 177Lu, 153Sm, 123I, and 111In; biological response modifiers or biological response modifying drugs, such as methotrexate, adriamycin, and lymphokines including interferons; toxins, such as ricin, abrin, and diphtheria; heterofunctional antibodies, that is, complexes formed by conjugating heterotype antibodies with each other, which are able to bind both to cancer cells and to effector cells (e.g., killer cells such as T cells); and natural, that is, non-related or non-complexed antibodies.

The pharmaceutical composition according to the present invention may additionally include a pharmaceutically acceptable carrier. As used herein, the term “pharmaceutically acceptable” refers to a physiologically acceptable composition which, when administered to human beings, will generally not cause allergic reactions, such as gastrointestinal disturbance, dizziness, and similar reactions. Examples of the pharmaceutically acceptable carrier comprise carriers for oral administration, such as lactose, starch, cellulose derivatives, magnesium stearate, and stearic acid, and carriers for parenteral administration, such as water, suitable oil, saline solution, aqueous glucose, and glycol. A stabilizer and a preservative can additionally be used in the present invention. Suitable stabilizers comprise antioxidants, such as sodium bisulphite, sodium sulphite and ascorbic acid. Suitable preservatives comprise benzalkonium chloride, methyl- or propyl-paraben, and chlorobutanol. Other pharmaceutically acceptable carriers can be found in the following literature: Remington's Pharmaceutical Sciences, 19th ed., Mack Publishing Company, Easton, Pa., 1995. The pharmaceutical composition according to the present invention can be formulated into a suitable form together with the above pharmaceutically acceptable carrier according to any method known in the art. Namely, the inventive pharmaceutical composition can be formulated into various parenteral or oral forms according to a conventional method. The parenteral dosage formulations typically include an injection formulation, preferably, an isotonic solution or a suspension. The injection formulation may be prepared using a suitable dispersing agent, wetting agent or suspending agent according to any method known in the art. For example, the injection formulation can be prepared by dissolving necessary components in saline or buffer. Also, the oral dosage formulations include, but are not limited to, powders, granules, tablets, pills and capsules.

The monoclonal antibody formulated as described above may be administered in an effective amount by various routes, including oral, transdermal, subcutaneous, intravenous and intramuscular routes. The term “administration”, as used herein, refers to the introduction of a predetermined material into a patient using any suitable method. The material may be administered via any of the common routes, as long as it is able to reach the desired tissue.

As used herein, the term “effective amount” refers to an amount sufficient to affect prevention or treatment when administered to a patient. The dose of the pharmaceutical composition of the present invention may differ depending on various factors, such as disease type and severity, age, body weight, sensitivity to drugs, type of current therapy, mode of administration, target cell, etc., and may be easily determined by those of ordinary skill in the art.

The pharmaceutical composition of the present invention may also be administered in combination with existing pharmaceutical agents, sequentially or simultaneously with the existing pharmaceutical agents, and in single dose or multiple doses. Preferably, with all of the factors taken into account, the minimum dose required to achieve the maximum effect without side effects can be administered. An effective dose ranging more preferably from 1 to 10000 μg/kg by weight/day and yet more preferably 10 to 1000 mg/kg by weight/day can be repeatedly administered several times a day.

Hereinafter, the present invention will be described in detail with respect to Embodiments. However, these Embodiments are intended to describe the present invention in further detail and should not be construed as limiting the scope of the invention.

### Embodiment 1

Animals and Drug Treatments

MDMA ((±)-3,4-methylenedioxymethamphetamine hydrochloride, CAS #92279-84-0, purity ≧99.9%) was obtained from the Reproductive and Development Toxicology Division of the Toxicological Research Department, The National Institute of Toxicological research, Seoul, Korea. MDMA was dissolved in isotonic saline at doses of 20 mg/kg at volumes corresponding to 3 ml/kg, and was administered orally (p.o.). Male and female C57BL/6N mice (6 weeks), purchased from the Orient Co. Ltd., were kept under SPF-conditions. All mice were acclimatized in the laboratory according to the ‘Guide for the Care and Use of Laboratory Animals’ (US NIH Publication No. 86-23, 1985 Ed.). To investigate the postnatal effects of MDMA in the F1 generation, we designed the following experimental protocols (FIG. 1). In this experiment, gestation day (GD) 0 was defined as the day when positive evidence of mating was first detected (copulatory plug in the vagina) and postnatal day (PND) 0 was defined as the date of birth. The mice were mated in a separate cage and recognized as pregnant on the day (GD 0) when vaginal plugs were detected. These females were given a single daily oral dose (20 mg/kg) of MDMA or vehicle from GD 6 through PND 21 (3 weeks after delivery). Control animals received the vehicle at the same dose volume (3 ml/kg). Following delivery, the dams (F0 generation) and their offspring (F1 generation) remained together until the completion of weaning on PND 21. F1 male and female mice were sacrificed at 11 weeks, and the brains were removed and placed on ice. Brain regions where then dissected free, frozen on dry ice, and stored in a LN2 tank until further analysis. The overall procedure of the experiment of the present invention is shown in FIG. 1.

### Embodiment 2

RNA Isolation from the Brain Tissue

Total RNA was isolated from the brain tissue using the TRI reagent (Molecular Research Center, Cincinnati, Ohio, USA) <according to the manufacturer's instructions (Life Technology, Rockville, Md., USA). Briefly, the tissue were frozen in liquid nitrogen, pulverized, homogenized in 1 ml of TRI reagent, and incubated for 5 min at room temperature; 200 ml if chloroform was added, vigorously mixed, and incubated for 3 min at room temperature. The samples were centrifuged at 1000 rpm for 20 min, aqueous phases were transferred to fresh tubes with an equal volume of isopropanol, and incubated on ice for 30 min. After centrifugation at 14000 rpm for 15 min, RNA pellets were washed in 75% ethanol and dissolved in RNAse-free water. RNA qualities were assessed using the Bionalyzer 2100 (Agilent Technologies, Palo Alto, Calif., USA), and concentrations were determined using a ND-1000 spectrophotometer (Thermo Scientific, Wilmington, Del., USA). Samples with a minimum 28S/18S ratio of >1.6 were selected for further study.

### Embodiment 3

Mouse Genome Survey Microarray

A 24-mer oligo internal control probe (ICP), co-spotted at every feature with a 60-mer gene expression probe on the microarray, was used. Digoxigenin-UTP labeled cRNA was generated and linearly amplified from 1 μg of total RNA using an Applied Biosystems Chemiluminescent RT-LVT Labeling Kit, v2.0. Array hybridization, chemiluminescence detection, image acquisition, and analysis were performed using Appliedi Biosystems Chemiluminescence Detection Kits and an Applied Biosystems 1700 Chemiluminescent Microarray Analyzer, according to the manufacturer's instructions. Images were auto-gridded and chemiluminescent signals were quantified, corrected for background, and spatially normalized.

### Embodiment 4

Expression Profiling Analysis of DNA Microarray Data

Applied Biosystems Mouse Genome Survey Microarray expression data were analyzed using GenPlex software (Istech Inc., Korea). For primary data filtering, spots with a SNR (Signal to Noise Ratio) <1.5 were excluded, and the remaining filtered data were used for further analysis. Quantile normalization was used to normalize data. Hierarchical clustering of log ratios was performed using Cluster and TreeView 2.3; Euclidean correlation, median centering, and complete linkage were used for all clustering applications.

To maximize the prenatal exposure to MDMA, the F1 generation was indirectly exposed during pregnancy (GD6) and up to three weeks after birth (PND21). Female and male offspring were then sacrificed at 11 weeks and their brain sections were prepared to analyze expression profiles of the cerebral cortex.

As a result of the analysis, the cerebral cortex of the mice associated with memory and learning was considerably affected by MDMA. As shown in FIG. 2a, gene expression of 13,848 genetic elements resulted in two major clusters. This showed that there existed distinct transcriptomic configuration of the male and female cerebral cortex. In addition, there were two more distinct sub-clusters within the first two major clusters. Each sub-cluster was then distinguished by a different molecular signature; the prenatal exposure group (MDMA, 20 mg/kg daily single dose ingested) and vehicle group, of both the male and female pups.

Moreover, both male and female offspring of the MdMA treated dams, clearly showed transriptomic changes in the F1 cerebral cortex when each gender was reanlzyed separately (see FIGS. 2b and 2c).

Therefore, prenatal exposure to MDMA after maternal ingestion during pregnancy and lactation affected the transcriptomic configuration of the cerebral cortex among offspring, and this change was permanent during development of the F1 generation.

Furthermore, to identify the molecular signature associated with prenatal exposure to MDMA in the mouse cerebral cortex, the present inventors analyzed the differential gene expression between a group exposed to MDMA and a vehicle using Welch's T-test and fold-change analysis. The Welch's T-test (P<0.05) and a 1.5 fold change cut-off were used to identify outlier genes.

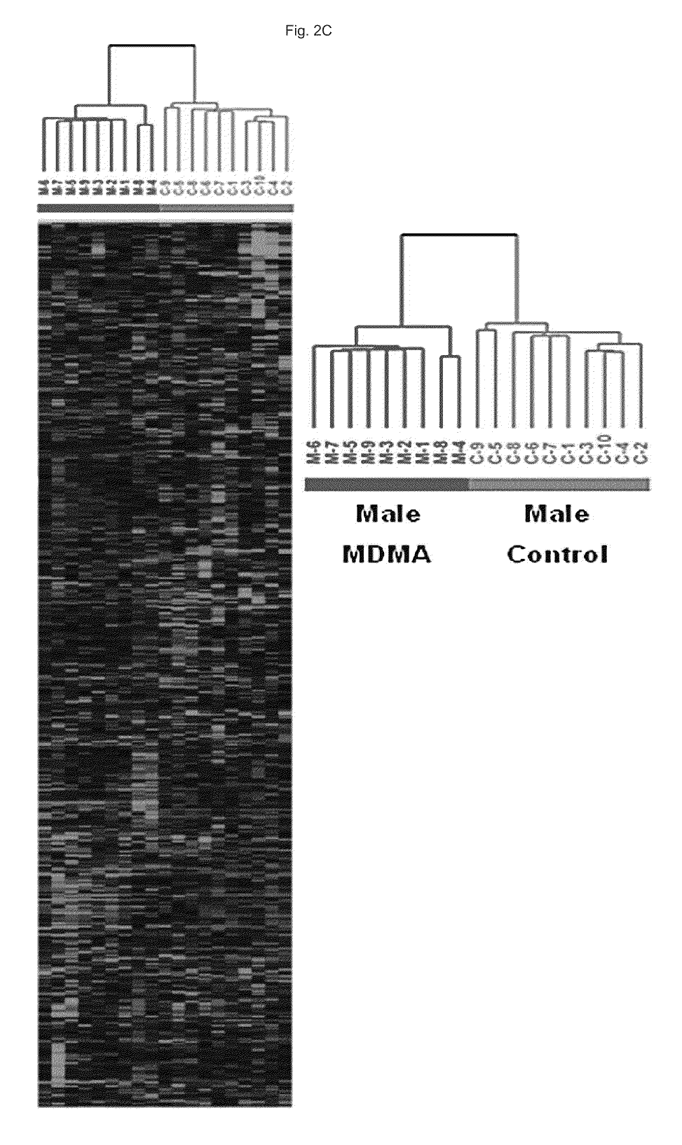

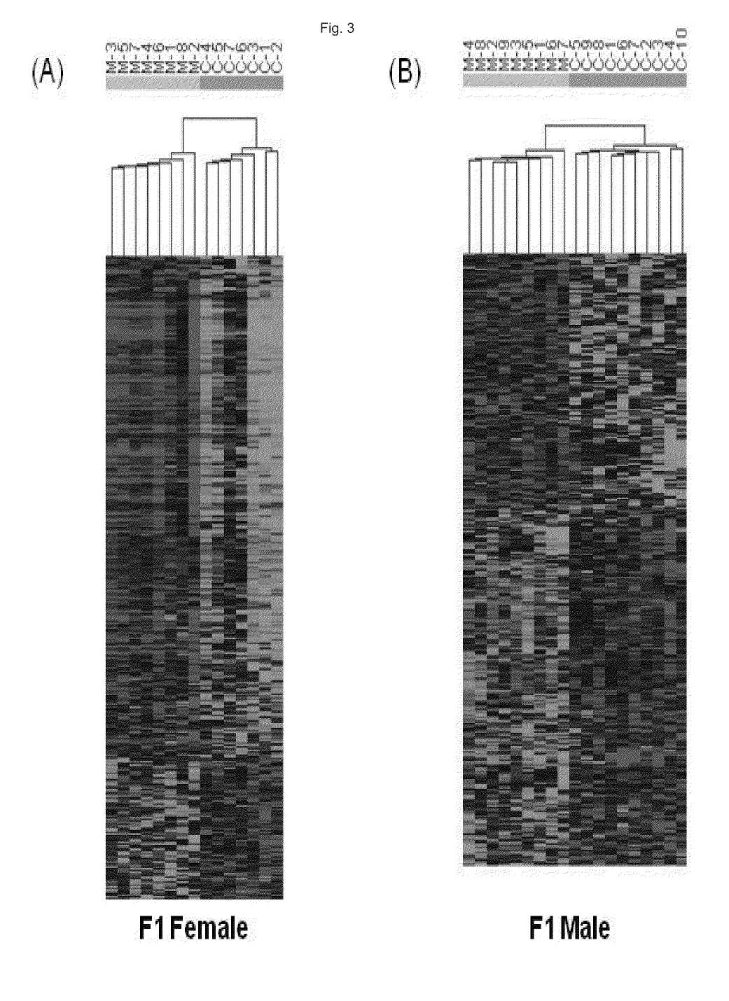

As a result, 1,784 genes (female group) and 804 genes (male group) were identified as differentially expressed genes after prenatal exposure to MDMA. Of these MDMA characteristic signatures, 1,385 genes were up-regulated (i.e., over-expressed), and 399 were under-expressed in the female cerebral cortex (see FIG. 3a). Similarly, 335 genes were up-regulated and 469 were under-expressed in the male cerebral cortex (see FIG. 3b). The relative expressions are shown in the heat-map in FIG. 3.

Moreover, the present inventors selected common genes from among the 1,784 genes (female group) and 804 genes (male group) that show differential expression after exposure to MDMA, and listed these common genes in the following Table. As the genes listed in the following Table showed abnormal expression as compared to normal samples, it was found out that these genes can be used as markers for predicting or diagnosing brain disease caused by brain injury.

| | | | GenBank | Expression |
|---|---|---|---|---|
| No. | Probe ID | Gene Name | accession # | change |
| 1 | 299424 | unknown(Sequence No. 1) | AK033066.1 | down- |
| 2 | 305559 | unknown(Sequence No. 2) | | regulation |
| 3 | 328759 | fibroblast growth factor 20 | NM_030610.1 | (under- |
| 4 | 354854 | RIKEN cDNA 4930451A11 gene | AK015436.1 | expression |
| 5 | 355052 | unknown(Sequence No. 3) | AK037760.1 | compared |
| 6 | 355535 | cytotoxic T lymphocyte-associated | AY034578.1 | with |
| | | protein 2 beta | | control |
| 7 | 361748 | T lymphoma oncogene | NM_011601.1 | group) |
| 8 | 368174 | RIKEN cDNA 2010323F13 gene | NM_177157.2 | |
| 9 | 392437 | RIKEN cDNA 4930566F21 gene | AK016234.1 | |
| 10 | 401554 | ribosomal protein S2 | AK004568.1 | |
| 11 | 424366 | unknown(Sequence No. 4) | | |
| 12 | 428440 | otogelin | NM_013624.1 | |
| 13 | 432053 | RIKEN cDNA 4933407A17 gene | AK016710.1 | |
| 14 | 433675 | similar to T-cell receptor alpha chain V | M23096.1 | |
| | | region PHDS58 precursor | | |
| 15 | 460185 | predicted gene 3364 | AK090251.1 | |
| 16 | 478590 | ring finger protein 149 | AK040602.1 | |
| 17 | 497466 | cyclic nucleotide gated channel alpha 2 | NM_007724.2 | |
| 18 | 513268 | RIKEN cDNA C030016D13 gene | NM_175354.2 | |
| 19 | 515525 | unknown(Sequence No. 5) | AK053269.1 | |
| 20 | 520252 | unknown(Sequence No. 6) | | |
| 21 | 524988 | early growth response 1 | NM_007913.2 | |
| 22 | 525785 | hypothetical protein 9130430E04 | NM_183203.1 | |
| 23 | 530939 | RIKENcDNA9330176C04gene; | NM_175420.1 | |
| | | solutecarrierfamily 22 | | |
| | | (organiccationtransporter), member13 | | |
| 24 | 566320 | unknown(Sequence No. 7) | | |
| 25 | 615742 | olfactory receptor 1350 | NM_146389.1 | |
| 26 | 749804 | RIKEN cDNA 1200002N14 gene | NM_027878.1 | |
| 27 | 762310 | RIKEN cDNA 4833417J20 gene | AK014716.1 | |
| 28 | 781797 | olfactory receptor 704 | AY317825.1_{—} | |
| | | | CDS_1 | |
| 29 | 783471 | unknown(Sequence No. 8) | AK037520.1 | |
| 30 | 824655 | unknown(Sequence No. 9) | AK037572.1 | |
| 31 | 838496 | RIKEN cDNA A430057L12 gene | AK078093.1 | |
| 32 | 841082 | RIKEN cDNA 9330198N18 gene | AK020395.1 | |
| 33 | 847317 | myogenic differentiation 1 | NM_010866.1 | |
| 34 | 850104 | gene model 44 | XM_907821 | |
| 35 | 910205 | RIKEN cDNA F630021I08 gene | NM_178743.2 | |
| 36 | 932668 | early growth response 2 | NM_010118.1 | |
| 37 | 298812 | RIKEN cDNA 1700007H16 gene | NM_027945.1 | up- |
| 38 | 334928 | matrix metalloproteinase 15 | NM_008609.2 | regulation |
| 39 | 338997 | Ras association (RalGDS/AF-6) domain | NM_175445.3 | (over- |
| | | family 2 | | expression |
| 40 | 339413 | cDNA sequence BC020354 | NM_198110.1 | compared |
| 41 | 357505 | high density lipoprotein (HDL) binding | NM_133808.2 | with |
| | | protein | | control |
| 42 | 357970 | cyclin-dependent kinase 19 | AK019224.1 | group) |
| 43 | 370504 | potassiumvoltagegatedchannel, | NM_008420.3 | |
| | | Shab-relatedsubfamily, member1 | | |
| 44 | 391067 | kelch repeat and BTB (POZ) domain | NM_001164493.1 | |
| | | containing 9 | | |
| 45 | 396150 | transmembrane protein 120B | BC022593.1 | |
| 46 | 398003 | phosphofructokinase, muscle | NM_021514.2 | |
| 47 | 444821 | unknown(Sequence No. 10) | | |
| 48 | 463085 | breast carcinoma amplified sequence 3 | NM_138681.2 | |
| 49 | 466313 | POU domain, class 3, transcription | NM_011141.1 | |
| | | factor 1 | | |
| 50 | 471089 | RIKEN cDNA B930093C12 gene | NM_172945.1 | |
| 51 | 472358 | sterile alpha motif domain containing | BC049740.1 | |
| | | 4 | | |
| 52 | 475067 | Rho guanine nucleotide exchange factor | NM_027144.1 | |
| | | (GEF) 12 | | |
| 53 | 488058 | cDNA sequence BC052066 | NM_172761.1 | |
| 54 | 491436 | fuzzy homolog (Drosophila) | BC045601.1 | |
| 55 | 498825 | SEC5-like 1 (S. cerevisiae) | NM_025588.2 | |
| 56 | 501289 | presenilin 1 | NM_008943.1 | |
| 57 | 526436 | insulin-like growth factor binding | NM_010517.2 | |
| | | protein 4 | | |
| 58 | 531607 | unknown(Sequence No. 11) | | |
| 59 | 540378 | RNA binding motif protein 8 | NM_025875.1 | |
| 60 | 544433 | RIKEN cDNA A830097P10 gene | NM_173780.2 | |
| 61 | 555234 | protein phosphatase 5, catalytic subunit | NM_011155.1 | |
| 62 | 555859 | BMS1-like, ribosome assembly protein | NM_194339.1 | |
| | | (yeast) | | |
| 63 | 559958 | gene rich cluster, A gene | NM_013533.2 | |
| 64 | 563270 | unknown(Sequence No. 12) | AK043558.1 | |
| 65 | 563705 | RIKEN cDNA 2210009G21 gene | NM_028834.1 | |
| 66 | 574782 | zinc finger, DHHC domain containing 14 | NM_146073.2 | |
| 67 | 597728 | RIKEN cDNA 6430548M08 gene | NM_172286.2 | |
| 68 | 602012 | dishevelled associated activator of | AY426536.1 | |
| | | morphogenesis 2 | | |
| 69 | 606287 | 3-monooxgenase/tryptophan 5-monooxgenase | NM_018871.2 | |
| | | activation protein, gamma polypeptide | | |
| 70 | 607746 | RIKEN cDNA 9330161C17 gene | NM_172614.2 | |
| 71 | 638437 | histocompatibility2, Tregionlocus9; | NM_010399.2 | |
| | | histocompatibility2, | | |
| | | Tregionlocus22; histocompatibility2, | | |
| | | Tregionlocus17; histocompatibility2, | | |
| | | Tregionlocus10 | | |
| 72 | 658280 | double C2, alpha | NM_010069.1 | |
| 73 | 668016 | RIKEN cDNA D930014A20 gene | NM_178811.3 | |
| 74 | 669220 | zinc finger, FYVE domain containing 20 | NM_030081.2 | |
| 75 | 675690 | RIKEN cDNA 2310075C17 gene | AK010164.1 | |
| 76 | 703524 | synaptotagmin 12 | NM_134164.2 | |
| 77 | 734020 | myosin XVIIIa | NM_011586.1 | |
| 78 | 734899 | wingless-related MMTV integration site | NM_009524.2 | |
| | | 5A | | |
| 79 | 746773 | RIKEN cDNA 2610511O17 gene | NM_152817.2 | |
| 80 | 757556 | XPA binding protein 2 | NM_026156.1 | |
| 81 | 769898 | UDP-N-acetyl-alpha-D-galactosamine: | NM_139272.2 | |
| | | polypeptideN- | | |
| | | acetylgalactosaminyltransferase2 | | |
| 82 | 829347 | RAD52 homolog B (S. cerevisiae) | NM_025654.1 | |
| 83 | 849739 | peptidylprolyl isomerase (cyclophilin)- | NM_026845.1 | |
| | | like 1 | | |
| 84 | 858428 | toll interacting protein | NM_023764.2 | |
| 85 | 894752 | ets variant gene 5 | NM_023794.2 | |
| 86 | 896014 | eukaryotictranslationinitiationfactor2, | NM_012010.2 | |
| | | subunit3, structuralgeneX-linked | | |
| 87 | 898183 | glucose phosphate isomerase 1 | NM_008155 | |
| 88 | 917144 | sodium channel, voltage-gated, type I, | NM_011322.2 | |
| | | beta polypeptide | | |
| 89 | 923175 | TRIO and F-actin binding protein | NM_138579.2 | |
| 90 | 925691 | mitochondrial solute carrier protein | NM_030054.2 | |

### Embodiment 5

KEGG Pathway Analysis

To identify the major pathways of the biological processes in which these 1,784 genes in the females and 804 in the males participants, the present inventors analyzed molecular pathways associated with differentially expressed genes exposed to MDMA by using the pathway mining tool in the Kyoto Encyclopedia of Genes and the Genomes (KEGG) pathway database (www.genome.jp/kegg/).

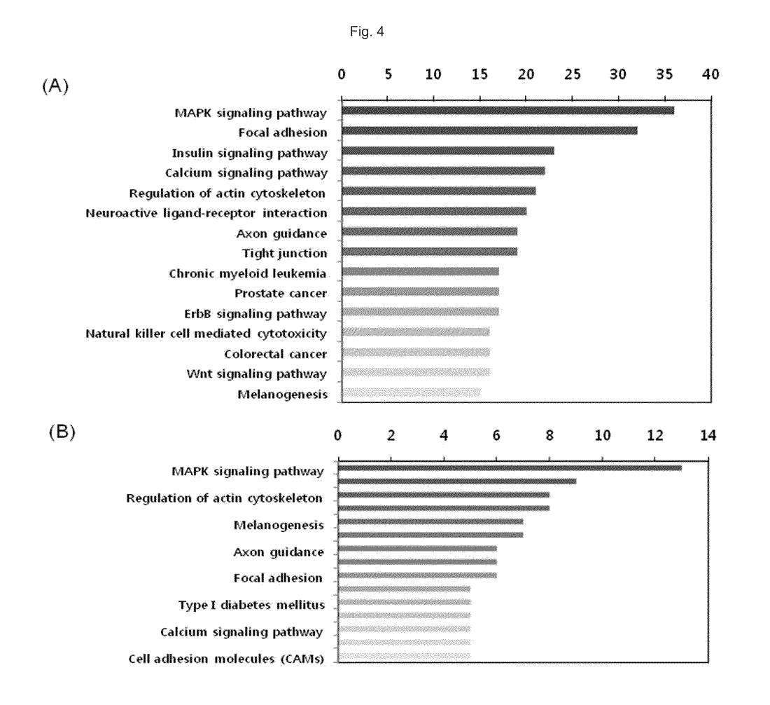

As a result, the present inventors identified the most common 15 pathways mapped from 1,784 F1 female MDMA-specific molecular signatures and 804 F1 male MDMA-specific characteristic molecular signatures (see FIG. 4). A large number of genes were mapped to four major pathways. Among these 15 biological processing pathways correlated with the F1 MDMA associated genes, nine were identified as common pathways in both the F1 males and females, and included the following: the MAPK signaling pathway, focal adhesion, insulin signaling pathway, calcium signaling pathway, regulation of actin cytoskeleton, Wnt pathway, neuroactive ligand-receptor interaction pathway, axon guidance and colorectal cancer signaling.

Although the invention has been described focusing on the preferred embodiments, those skilled in the art will appreciate that the invention may be carried out in modified forms without departing from the essential characteristics of the present invention. Therefore, the above embodiments should be construed in all aspects as illustrative and not restrictive. The scope of the invention should be determined by the appended claims and their legal equivalents, not by the above description, and all changes coming within the equivalency range of the appended claims should be construed as being embraced in the invention.

### SEQUENCE LISTING FREE TEXT

As regards the use of nucleotide symbols other than a, g, c, t, they follow the convention set forth in WIPO Standard ST.25, Appendix 2, Table 1, wherein k represents t or g; n represents a, c, t or g; m represents a or c; r represents a or g; s represents c or g; w represents a or t and y represents c or t.

1.
gagaaggccagcaacgttcctgactgcacgcaggagtctattagtctattagtctacagcttgggcaaatggaag
acttgtcccagcctcagtttctccatccacaaacagggacgatgataatgatacgcacccagagagctggatgag
gatcaaatgggctttttggcacttagaggcgctgtgcagcaagcaggcatagctggcttacatcttcaccaccaa
ccgccctctcccaaagacccactccagaaacctgcccccgaccgaggcgcccatctcacctggcttcgtacacag
gtaggagtaaaagccctcggacttgagcatgatgagcagcgaaccccagcccaggaggactgcggagaaaaggag
gttttccaacacagcggtgcaggccatccaccagcggcgccgatgggcagtggccagggtgggcgccatggtgcg
gtgtggcggtggtcgggctccggttctgcagacacctgcgcgcagacctcagcttcagcggtggctctacccaac
ccgtgtagccgctggcgcacagcctgtcagtcccttgcagcccaggctataggtgcccgccgcaaaagaaaagaa
ccctagccctgtcagggacctgggccaccccgtacccgcagctacctggtaacgctaccttaggccacagtggag
ccccgctggccaagaggaaaggcttctcccttagttcactcggaggtaaactgaggcatggcagctggaaaaggg
ctaattatgctcaaggacgtggagcgggtcctgatcccaagcaagactgaacagccactttgggggccggaaagg
gaggcagctcgtagtaaggtcacctgagcagaggctcacctgagagctgccactcccagggaccctgacaggctc
ccgcccgcccgcactcgagacccttccttagtgcttgttccttcaaccacaaaatcagcacagggagccccgaaa
cggcccgcccacggcgctttcattggcgggaccgcacaggaactgccgcgccattggttagtgtttaggtcgcta
ggcgctaccgccacgcgaaccccgcatgggagaggcgaagccggttcaaaccggcggcgcgagccaaccgcgcga
cccccgtgtcgggctgttaccccggcgattagcccgtttccatagaagcggatcccgtccctctgtcctcctggc
ctttttcgttacgtctccctcccggtgtcgtccctctgacaagtatgcaccttccctccaacccctgcctgcgag
cggcttcaggttctcgcaccctcgtctgagacccggatcctgggagtccagctcacccggctcctaccagtgtgt
atcgggcccgcccggcgccttttctgtcccctttatttccagcccttttattcctatcctaagatgaaaaacact
gccgcccgttgattggctgagtggggctggaaacaggcgtcggccaatcagcagctacccacccgccccccggcg
agcagaagccggttccggccggacttggagacaaacaagaggaatgggggggctggggggcggctcgggcaggcc
cagctctacccagttaaactggagtgcccctcgacccggtgaccgccgcgggcatcgccccacgcccttctcgtt
gccctcttccttgtcaagttcatgggtcagcgtttcagggagtcttggtccacagcgagctgggctccaagcacc
gtgcggttcggcccgcagtatagaccaccgttctcgcccggagagaaccagggccggatgctccaactccgcagg
ccattaccgcccttttgtgctgtttcccggcctgctccttgggctttgtgtccgggctcctcgctgcctagcccc
cgttgtcctatccaggcgctgtctacgggtatcagaacaccccgacccaggtcgtggcataattaccgcatccct
gctagatctcccgactcaccgggtccttgcctggaatcaggtgcctcctgcctggtctttaatcggtgtccctta
agggtctgtagtcaggattttggactaagagcccctggtcctccagttgtcgccatccccatcccgcgccctctt
ttctcggtgccttcagagaccggtcagtgttcaggccggtcaccttggcatgccctgccctcccactttcccaaa
cgactctttcatgtggctggcacactttctcgtccacccttcttcatgctgtgagcaatcccgagcacctagtta
gtctaagtctgactttggagagcgcgatagatcactaggggtccaaagagctccgtcctttctgtatcatcagcg
gagctgcgctgctctgaggacctgcttggacgtggaatggagcctcagcacgaaagatttggctgtccgtccgag
tctccctgacgagaggcactaacctcccctcagaaaacactcggtatccctcagattctccacacttcaagagcc
ggcttgctttagagctaagggtgtccccagcagcctgactcagcttccctcagctcgagtgcgccccctggtgcc
cagaaccttcgccagccattctgctagtgcatgctctcccagacttggtctaggcttgctggtgggtttgggggc
ctgatgacctactgtgcagaaatctcataagctccaaattggacctactatctgccaatagattaagcccttcaa
tccttttcctaagcaatctgcttgaggaccttctccagacatatatctaaaatgttccctccccctcttcaaata
aatagtgcccccttctccctc (SEQ ID NO: 13) describes the adult male corpus
striatum cDNA, RIKEN full-length enriched library, clone: 7630402D21
product: unclassifiable, full insert sequence. NCBI reference sequence is
AK033066.1.
2. atggctcccttgaccgaagtcggggctttcttgggcggcctggagggcttgggccagcaggtggggtcgc
acttcttgctgcctcctgcaggggagaggccaccgctgttaggggagcggcggggcgcgttggagagggg
cgcccgcggcgggcccggttccgtggagctggcgcacctgcacggcatcctgcgccgccggcagctctac
tgccgcaccggcttccacctgcagatcctgcccgacggcaccgtgcagggcacccggcaggatcacagtc
tcttcggtatcctggaattcatcagtgtggcagtgggactggtcagtatcagaggtgtggacagtggcct
gtaccttgggatgaatgacaaaggagaactttatggatcagagaaattgacttctgaatgcatcttcagg
gaacaatttgaagagaactggtataatacctattcgtccaacatatataaacatggaaacacgggtcgca
ggtattttgtagcacttaacaaggatggaactccaagagatggtgccaggtccaaaagacgtcaaaagtt
tacccactttttaccaagaccagtagacccagaaagagttccagaattatacaaagacctactgatgtac
actggatga (SEQ ID NO: 14) describes fibroblast growth factor 20
(Fgf20), mRNA. Gene name is Fgf20 fibroblast growth factor 20. NCBI
reference sequence is NM_030610.1.
3.
gcctttgggacgggaatagcgtgaggacaagatgaggcccagagtctcctggtttcagtggcagatctgtcatga
ttcttcaccggaggatttagatttaataaggcttacaagattaggattttagttgctgcacccagtgattgagtt
accgttgtttctgaactaggtttgtgttgtgttttccttcacgtggtgactcatctgggtccaagagaggaaggt
atggcggcaaagcgtgggtttgcctgatgtgcaccacaaagaccgtgggggatttgaagcacggggttggcatgg
tagcgacccgccagtaggaatttaataagctgggtgaaaacattgtcgagttatgaatcaaagtctccacaagtt
aaagacaggtcggccattcccgccagtgtctggccggccaggccactgggacagaggcacaagtgaaggacatga
cggttcttttttttttaatatttcctgcaacatgtggcaatatatgaagtgccagagggcacagctcgcatgaag
gcaggtcctcatctctgggactcgtttgttgtgttttaaagttttttagaaaagttataaagaaaatccatctac
atgtatcattttgataaaaataaaaaatttaatgttc (SEQ ID NO: 15) describes adult male
testis cDNA, RIKEN full-length enriched library, clone: 4930451A11
product: unclassifiable, full insert sequence. Gene name is RIKEN cDNA
4930451A11 gene. NCBI reference sequence is AK015436.1.
4.
gaggaaataacatatataattttaaattttaaccatgatgtagaacacaagtgtagtcatgaacgtatttgctgc
cttgtatgtttgttgtaggagacaggtgtttgagaagtggcatacggtcttcaagaggaatgagtcaaaggatag
attgtggataccttggaggccacaggtttgttaccgctttgctgagttatatgggtttgtcttctcaagggggtg
aggaacagaaaacatcctttaccatctgaacactatgaaaccgattagatgaacagacactggagagaatgtcag
atactattctgaacaatggttgctggtgccttcagtctgtacagcaagagcagaccatagaaacaaactgatttt
cttgaagttaaaggagtgtgttagtctcctggtgctgctataagagaccaccacagcctgggttattcctcacac
tggcagctctgttggatgaaggccccacccttttacctcacctaaaacttcatcaccagaaaagtcatattggaa
tataaggcttgtgcataggagttttagaggagaaatagctaagtctataactgagaaattcaggaagtccacact
gtcctggaaggctcggcaaggcctgcagagttcctgatttttgtctcatgtctatcaagctccaaggtggtgctg
atgctgttgctctgggaactgtacttggaggattgcttttagattgcgggggtgggatgggggtgtcacccttga
gaggtgtgagacaggggtatggcagaaacagaaggcagtgggacgttctggagactttcttggttcagcttacca
agttatgtttagtcccgatttgagacattcaaagaggtttgattgaagctccatcactgacattgtctgggaaat
aagtgctaat
tttattaacagtcttactccctggaatattcagaattttggtcaacttttgtccccctgcctttgccacttccga
tcttagtctcctgtcccagtctgatgcgtgcttctgaggctctggctcctctcagtgcatacgtacacaggggat
tcccgatcctctcctcttctgttctgtcatgtgcttctctttacaagtagcctttcttctgctcgctctctctct
ctctctctctctctctctctctctctctctctctctccctccctccctccctccctccccagaataaacagactt
gcatagctgtcctctttcttgtattcttcacaaaaggctgacacgttcccttgtacttggtatattttagaaact
ccaccatattggctggtagacagcagcttccctgtcacaggtgtgttgtgttctgatctgtgggccattctgttt
gcaaccactgccctccgtttggagcttaaaaggaggctcccagtattctgaagtttaagtgatgccgcagagcat
aaccctgtgcctatgtattccacattactaaggtatatcttcagagtacatttctagttcttttgggtagagtgc
tgttgatgtctgtctccactccccaaattcctgccatgctcttgtctgtggactgggttcatgatgtaaggatgc
cttattaaaataatgaagtttagaaacaatagataatggtggtagtgaggagaactccaggggaatctgtgtcct
tggttcgtttctttggtttttacagcagccttaatttccttcctggtcgcgtctgctgcttctccagaagcactg
tttatgtgagtgtgccatttgccttgctgttgccttgcaaccttctgtgtaattgggtgttaccatgaccatccg
tatttgaata
ttcatccttttggcacttggtatttgattttttttttcccctctgtgagtgacttcatctcgtttgtctgtgtca
tttggtctcctccttttcacaagatttcttaagtctccttttactcccgtgataggtgccatagactcgggtggg
ctgtgattagtgcccacagactcatggacggtggaggccctgttggtttgtggtgtttattatgcacagtcattc
gaagtctctccttagaatattggaagccatgtgtggctttgtttgtacttcctgtggttttaagctttgagaaag
gagagtttgggatttagggttttgcagttatcctcctggctcagaagttgtaaaggaacctttcataatcaaggt
tttcgtatgctaatctaggtcacatactctcctcctgtagggctctgtgtctaagtctttgcattacatatacat
gtacatgtacatatacaagcatgcagatgtagatgttcttacaatgacctagtgcagacctagtttgccaagctg
tttaccgtaggccacggataagcagactcttctttaaaaggtcattttttaaatttttgaagttacagaaaccaa
ataatctttgtgaaattcttgctcctccctatccttttcccctcttctttcccctcccc (SEQ ID NO: 16)
describes 16 days neonate thymus cDNA, RIKEN full-length enriched
library, clone: A130047I21 product: unclassifiable, full insert sequence.
NCBI reference sequence is AK037760.1.
5.
ctggacaacaaagttctggtttctatctgtgaacagaagctgcagcacttcagtgctgtcttcctgctcatcctc
tgcttgggaatgatgtcagctgctccatcccctgatccaagtttggataatgagtggaaagaatggaagacaaca
tttgcaaaagcctacagtctggatgaagaaagacacagaagactcatgtgggaggagaataagaagaaaattgag
gcacacaatgcagactatgagcggggcaagaccagcttctacatgggcctgaatcaatttagtgacttgactcca
gaagaattcaggacaaattgctgtggaagctcaatgtgtagaggagaaatggctcctgatttgcctgaatatgaa
gatttgggaaagaacagctatctgacacctggaagggctcagccagagtaacagttgtggcttgactggtaacaa
tatcatagtctaggagtgaagcaagcactctgcattgtcttgggagtcttctgtctcctggtatgggaggaatga
tgctgtagaggctcaacagcaggaagcagcccatcacagtgtccagcatgtgctgtgtgatatgcaaaactgaac
tcatataagatcaacactgtggtgtgaactctgaggcacagtcatactgagaaatcattgccaatttttagctct
gaagttccttaaatgattctcacaaggatctttagttttcactttaataagatgaatatatgccatgatgtcttc
aaattaaagctagatattaaattaaaa (SEQ ID NO: 17) describes cytotoxic T
lymphocyte-associated protein 2 beta precursor (Ctla2b) mRNA, partial cds.
Gene name is Ctla2b cytotoxic T lymphocyte-associated protein 2 beta. NCBI
reference sequence is AY034578.1.
6.
atgagaacccatctccttctctccagacctcagtcatctagaggacctgcggctgggcacagagcaagacacact
gaccttctggtgcttttagagtccccagcccctgttctctccactgttatgatgtgtctcctctggtggccagct
cctgccaagatcacttcacactctctcatccaattaacaaatcaatgcgtttgcctttactttgctccagggcat
atgtgtgtgtgggtgctcccaggccaagcctgcagtatggcactacagagcttcttccttgtgtctctctggcta
cttccctggcaaaacccagcacggtttttccttccttctctaatcaacagaggattctttgattcaatgtcaccc
ctccaatgcactcttgttctgtctgtggtcctagcctctcttgttcaatggttggctgtgagcatccatctctgt
atttgtaaggctctgtcagggcctctcaggagacagccatatcaggctcctttcagcatgtacttcttggtatcc
acaatagtgtctgggtttggtaactgtatatgggatgaatctccaggtggcacagtgagtggtctttcctttagt
atctcttctacacttttatctccatatttgctcatgtattttgttcttcttctaagaaggcacggtagtactatg
cccaattttctgaggaactgtcaaactgatttccagagtggttgtactagcttgcaatctcaccagcaatggagg
agtgtccttctttctccacatcctcgtcagcatctgttgccatctgaatttttgattttagccattttgactggt
gtgagatggaatctcagggttgttttgatttgcatttccctgatgactaaggatgtctttaactgcttctcagcc
attcgagatt
Cctcagttgataatcctttgtttagctctgtaccccatttttaa (SEQ ID NO: 18) describes T
lymphoma oncogene (Tlm), mRNA. Gene name is T lymphoma oncogene. NCBI
reference sequence is NM_011601.1.
7.
gagagcagggagtaacgggactgtggtcgcgggcagccccagccacgcaccatgccctatctgctcatcagcact
cagatccgtatggaggtgggccccaccatggtgggtgatgaacactcggatccggagctgatgcagcacctgggg
gcctccaagagaagtgtcttgggaaacaacttttacgaatactacgtcaacgaccctcctcgcatagtcctggac
aagctggaatgcaagggcttccgagtgctgagcatgacaggggtgggccagacgctggtgtggtgtctgcacaag
gaatgagctgcttgagctgacctgttcagagaccgccatgtgccctgcccacatccttgcatcactgtttgccct
gcctatctctttccaaattgtcaactcccttgcccaatactggtgggctgtgaacagcctaagggacctcgctcc
agctggttgggcacagcaggctcctgccctggtgctttcagcttcccccagctaggcctgaccctggctcttgct
gtaggccacatgctgaaggcaagccaacccgaggacgaggacagtccctggatcctacacagctaatccaagcca
ataaagggctctcacaaagtggttgttcctc (SEQ ID NO: 19) describes GTP
cyclohydrolase I feedback regulator (Gchfr), mRNA. Gene name is Gchfr GTP
cyclohydrolase I feedback regulator. NCBI reference sequence is
NM_177157.2.
8.
gacaactctccacgccaaccagagtagtagacgttacactcccctactctttaggcccgggccttcggaaagctg
ccaattcgcagcaagacaagttcagctgtaccaagtgtgcattaaggttgcccaggagccggcctcaagtgaccc
aggctagggttcgaggcttcgggaaggaaaggagtcagaggacagctcgagaaaaccacgcgcccggacccgcag
attccccagacaacttcctcccttcttcccgggcctgaggcgccactaccgcttctgcacggtgcacgtcacctg
tacaaaggtgtgtctttcgctgcaagaactgcaagtgtgcctggtggtccttccgagccgtctcagctttcacat
cctccagttccttctctaacatctggattttcatctcctggctcttctgtatggaggaaatgtgctccattgact
gaaactgctgctccaactgggactgcttgatttttccctggaagagttgtgtttgaagctcctcagtttgcttgg
cgaacctgagtgtcagttcttgtttcttcgaataatctccccaaggtcctgaacatactgatctcacagttgcgc
gtgtttctttttacactgctcactctgcttccttaagactctcaaaaagcagttcctctcagcgtccacacagag
agtgtcacgctgcagcagcctgctgtcttgcagcaggtgctcctgctggagcctggcctcttctatttttctgct
caggctctcaagtgcctccattgtcttgctttttacctccttctctaattctgtcagcctctctgacttgaaaag
ttcatttagaatagtaagatacgttgatggctgaggagaaccttttgaactctgggcaaacctggaggaagaaca
aaggaaatat
tcttttatcagttctctaggcacaagtgagaaaataatatctggaaactaaacctttgggttccatcatgcccaa
gaagggtcagtgcgggttagatttttgacaagttggtacattcaggaactatctgggaggaggaactgaatcgag
aaaagggactccatcagattatctgtagaacagacagacactctctctctcaaagatttatttatttttatatct
taagtacactgtagctgtcttcagacacaccagaagagggcatcagatctcattacggttggttgtgaggcacca
tgtggttgctgggatttgaactcaggacctccggaagagcagtcagtgctcttaactgctgagccatctctccag
cccctctgtctctcttcaaattaaagatagattgatgtgggtaggcccagcccttgagggtaaggtgacacccct
aggcaggtggtccttaactgaatacgaaagcagtccaagcaactgaacaatttatctctgtggcttctgcttcag
ttcctgcctccaggttcctgtcttgagctcctggccctgacttctttggatgatgaactgtgatacagaagaaaa
agatgaaataaaccctttctcaatg (SEQ ID NO: 20) describes adult male testis cDNA,
RIKEN full-length enriched library, clone: 4930566F21 product:
unclassifiable, full insert sequence. Gene name is 4930566F21Rik RIKEN cDNA
4930566F21 gene NCBI reference sequence is AK016234.1.
9.
gcttcttcttcttcgggagaacaccaaatggcggatgacgccggtgcagcgggagggcccggaggacccgggggc
ccaggattaggtggccgcggcggcttccgcggaggattcggcagcggtcttaggggccgtggtcgtggccgaggc
cgtggccgtggtcgaggccgcggggctcgtggaggtaaagctgaagacaaggagtggatccccgtcaccaagctg
ggccgtctggttaaggacatgaagatcaagtccttggaggagatctacctgttctccctgcccattaaggagtct
gagatcattgatttcttcctgggtgcgtccctaaaggatgaggttctgaaaatcatgccagtgcagaagcagact
cgggctggccagcggaccaggttcaaggtgacaggccgctgtggctctgtgctggtgcgtctcatccctgccccc
agaggcactggcattgtctctgctcctgtgcccaagaagctcctgatgatggccggtatagatgactgctacact
tcagccagaggctgcactgccaccctgggcaactttgctaaggccacctttgatgccatctccaagacttacagc
tacctgactcccgacctctggaaagagactgtcttcaccaagtctccttatcaggaattcacggatcatcttgtg
aaaacccacaccagagtctctgttcagaggagccaggctccagctgtggctaccacataagggtttttatatgag
aaagataaaagaattaagtctgctgaaaaaaaaaaaaaaaaaaaaaacaaaaaagaaacaacaaaaagagaaaaa
agaaaaaaagagacaacccgaaaaatagaagacgagtgtggggcggggggggggcgggggggcgcgccgctcggc
gacgtattat
aaattcatacgcatgtttgtataaatagagagagggtatatacgggcgccaccgccctctggcagcacatcctct
cccagcgggcggacgagacggtgcgcg (SEQ ID NO: 21) describes adult male lung cDNA,
RIKEN full-length enriched library, clone: 1200003H09 product: ribosomal
protein S2, full insert sequence. Gene name is Rps2 ribosomal protein S2.
NCBI reference sequence is AK004568.1.
10.
agcagagccctgggccaggctctctgattgcctggcaccctgatgggagcccgcatgcccagacgatgcttgctt
cttctctcttgcttttgtctacttagggttgagtccactgcagaggtgcagcaccaggcctcagcactcacatgg
aaaatcagtgcagagctgcaacaggagcctgctcctgagcccagccacacataccaggagatgtcccttgcagtg
gaggatgtcacaacagttatggaaggccaggaggctgaagccctagccgcatcggccatgtcttcgtgggaaaga
cggctccatcgcgccaagtgtgcaccgtcgtacttgttctcctgcttcaatgggggtgagtgtgtgcacccagct
ctctgtgactgcaggcgcttcaatgccactggaccccgttgccagttggtgtataatgtaggtcctgagcgggac
agcatctgccggacctgggggcagcaccacgtggagacgtttgacggcctgtactactacttttctggaaagggc
agctacacgctggtgggacaccacgaacccgagggtcagagtttttccatccaggtgcacaatgacccacagtgt
ggctctgcccactacacctgcccgaggtctgtcagcctcttcctctcaggtgaacgtgagatctgcctggccaag
gaagtcacccatggaggtgtaagggtccagctgccgcaggtggtggggggtgtgcagctgcagcagctggctggt
tacgtcatcgcgaggcatccgtcagccttcaccctggcctgggatggtatctcagccatttacatcaagatgagc
cctgagtttctgggctggacccatgggctgtgtgggaacaacaatgctgaccctcaggatgacctggtgaccagt
tacgggaagg
tgactgatgatgtaggggagtttgtacacagctggcaggagcaggtccccaacagccctccaggacctgtgacta
cttccctgcctcgcccaccatgcctgcagcagagcccagcatccatgcagggtgtgtatgagcggtgtgaagtac
tgttgaggcctcctttcgacacctgccatgcttatgtcagccctctgcccttcgcagccagctgtaccagtgacc
tctgccaatcaggaggcgatgaagccacttggtgccgagcactgatggaatatgcccgggcatgtgcacaggcag
ggcgccctctgcagggctggaggacccaactctcacaatgcactgtgcactgcaaagagaaggccttcatctata
acgagtgtattgcttgctgtccggcttcctgccaatcccgtgcttcatgtgtagacagtgagatcacctgtgtgg
atggctgctactgccccaatgggctgatctttgaggatggaggctgcatgtcaccagctgagtgtccctgtgaat
tccatggaaccctgcacccgcctggctctgtggtgacagaagactgcaatgcctgtacgtgcacagcagggaagt
gggtgtgcagctcttccgtctgtccagctgagtgctcagtgactggtgacattcacttcacgacctttgatggac
gccggtacacattccctgccacgtgtcagtacatcctcgccaagagccgttcctcaggcacatttacagtgacgc
tgcagaacgccccgtgtggcccgaaccaggatggggcctgtgtccagtccgtgtcagtgatcctgcaccaggacc
ctcggagacaggtgaccctgacccaggcaggagatgtccttctatttgaccagtacaagatcaccccaccctact
cagatgatgc
ttttgagatccggaggctgtcctccgtgttcctgagggtgaggaccaatgtgggcgtgcggatcctctatgaccg
ggaagggttgcggctttacctgcaagtggaccagcgatgggtggaggacaccgtgggcctctgtggcaccttcaa
cggcaacacacaggacgacttcctgtctccagtgggtgtacctgagagcacaccacagttgtttggcaattcgtg
gaaaaccctgtctgcctgtgtcccgctggtccctggctctttgctggacccctgcgatgtgcatctgcaagctgc
ctcctatgcactgcagtcctgcagtgtgctcacgggagagctctttgcaccctgctctgcgtatctgagcccaat
accctactttgagcaatgccgcagggatgcctgccgctgtgggcagccctgcctttgtgccacgctggcccacta
cgcacgactatgccggcgccatggactcccggttgacttccgtgcccatctgccagcctgtgcactgtcctgtga
ggccactaaagagtacagcccttgtgtgaccctgtgcgcaccaacatgccaggacctggccagccctgacgtctg
tggggctaacggaggtggcaacttcagcagggaggagtgcgtagagggttgcacctgcccaccggacacatttct
ggacacacaggctgacctctgtgtccccaggaaccaatgctcttgccatttccaaggagtggactacccccctgg
ggacagtgacatcccttccctgggccactgccactgcaaagatggggtcatgagctgtgacagccgagccccagc
tgctgcatgtccagcaggccaggtgttcgtgaactgcagtgagctgcacccagaccctgagctcagcagggagag
gacgtgtgag
cagcagctgctgaacctgagcatgccagcacgtggcccctgcctgtcgggctgtgcctgtccccagggcctgctc
agacatggggacgcatgcttcccaccagaggaatgtccatgtacctggaaggggaaggaatatttccctggggac
caggtagtctcttcctgccacacctgtgtgtgccagcggggctcgttccagtgcaccctgcacccctgcgcctcc
acctgtaccgcctatggggaccgacactaccgcacatttgacgggcttccgtatgactttgtaggggcgtgcaaa
gtgcacctggtcaagagtacatccgagctcagcttctctgtcatggtggaggatgtgaactgctatggctccggc
gtcatctgcaggaagtccatctccatcaacgttggcagctcactcatcatctttgatgacgactccggggatccc
agtcccgagagcttcctggatgagaagcaggctgtccacatctggagagcaggctttttcacactggtgcatttc
ccacgggaacacatcaccctcttgtgggaccagagaaccactgtgcatgtccaggcgggccctcagtggcagggt
cagttggtgggcctctgtgggaactttgacctgaaaaccatcaatgagatgcggaccccagagaacctggagtta
acaaacccccaggagtttggaagcagttgggctgcggtggagtgcccagatacccctgatcccggggacacttgt
gtcctgaatcctctccgagaaccatttgcaaggaaggagtgtggtattctgctcagtgaggtgttcgagacctgc
cacccagtggttgatgtcacttggttttactcaaactgcctgacggacacatgtggctgcagccggggtggcgac
tgtgagtgct
tctgtgccagtgtagctgcctatgcccatcagtgttgtcagcacggggtggtcattgactggcggacaccccgca
tctgcccatatgactgtgacttcttcaacaaagcgctaggtaagggtccctaccagctgtccagtgtggcggctg
gcggtactcttgtggccacgaaggcagtagacagtgacatagcccttgtgagggcagaggatctggcgccagggg
acatttcaagcttcctgctgacagctgctctgtacaaggccaaggcccatgaccctgacgtggtgtccctggagg
cggctgacagacccaacttcttcctgcacaccacagccaatggctctatagggctggctaagtggcagcgcgatg
aggccttccatcagcacgcttccttctcgctgcaccgggggacatggcaggctggcctggtggctctggagtccc
tggcaaagcctggctccttcctccactcatcaggccttgagctggcactgcgggcatacgaacacacggaggtgt
ttcgtggtggtgcgctcttccgccttctggatgccaagcctttgggggctgcctaccccacctgtgagtggcgct
acgatgcctgtgctagcccctgtttccaaacctgccgggacccacgggcaaccagctgccaggatgtgccaggta
gaagctgtgtccctgtgtgtcccactcctaaggtcctggatgaagtcacacagagatgtgtctacctggaagact
gtgtggagccagccccgcggggtcccacggagacccttggcaatgagactctggtacctggtcaagtgccaccca
ccaccagtgccgagcagcagcttccacaggggctccctggagcctctgcctacagccccgcccctgttccagtag
ccccacccac
ctcagccccaaacccaccaatggctgccacggaggggcaagcgccatctccaggatccacccaacccacactgca
gacacccctgggccttacaacatctaactttcctgctggccacacagaggccacagccagagaggaaggggctgc
cagccttctaaccacttctcatcccccagggttctcgtcctccctccctagttccctgcagatgcccacatctgg
cattgtatcaggtgcaacagagacaaccaaagtgaccataacctttacagggagtcccaacaccaccgtcgcttc
ccggtcaccccctatacctcgcttcccactcatgaccaaagctgtgacagtcccaagtcatgactcctttcctgt
taaaaccacacctctgcagccctcttggctatggagcctctcttccagacctatgacttcccttggagctacgtc
ctggccccccacctccccgggatcccacctgtcaactgcagtaactaaggtggcaaataagacaatgacatccct
gtctgtcctggcccagagcacttcaagttccagccagcccctggctgcagtgaccacagcacacagggctcctgc
cagtccccttgtcaccaagggcttggaggtggtgtcagccacagagaagggagaagctgggcacagccagcttac
agagttgccagtgtccccacccccaagcccagcccccatcgacctgcctcacccagcccaacataccaccacagc
ccctgggccttctgctctgtccccggggatcctggctgctggcagtccatccacaggtgctcacagaccgggggc
cacagctttagcttctctagagccaactcggccaccccaccttctctctggcctacctctggacaccagcctgcc
cttggctaag
gtgggaacctctgccccagtggccacacctggctccaaaggctatatccccacccctccacctcagcaccaggct
acaactttggctacggccatgactgtgagcccacttactcagtcactcagtctgacagtgccactcatgtctgct
gtagaggaacaggcacactcacctagccccaagcctccccaaggaacaggtatggctccagaccagatgctggga
gccacattgccaagctttggagcctcctctgtgattgctggggtgccccccacagtgtctgcggccccacggaag
agcaccacccagagagcagccattctatctaagaaggtgtctccacccaccctcatctctgactctgtccagggt
gggttcacagaactcacacctatagtgagccatactgttacccccttggcgactgaggctgaaggcccccgggct
ggcacagtgccactggtgcccacaacctactccctaagccgggtgtcagccaggacggcctcccgggaaggcccg
ctagttctgttgcctcagctggcagaggcctatgggaccccagcaggactccagccccaagaagatctaatgcgc
caggccactacggagcagtcagggcgctcagctccagcccagagtatagcagaggagtcgatggaggccgaagtg
aatacttctgccacctgtgtgcccatcgctgaacaagactgtgtccatcacatatgcctagaaggccagctgata
cgtgtgaaccagacccagcactgtcctcagggtgccgtacgccctcgctgtggggtcctgggcctcgctgtgcgg
gtgggcggggatcgctgctgcccacaatgggaatgtgcctgccgatgctcaatcttcccagacctgagctttgta
actttcgatg
gcagccatgcggctctgttcaaggaggccatctatgtcctcagccagagcccagatgaaaccatctctgtgcatg
tcctggactgcaagagtgccaacctgggacatctgaactggcccccattctgtctggtgatactgaatgtgactc
atctggcccaccatgtcagcatcgaccgcttcaacaggaaggtgactgtggactctcaagttgtgtggccaccca
tgagccgatatggcttccgaattgaggatacaggacacatgtatatggtccggacaccctcacacatccagatcc
agtggcttcacagctcaggactcatgatcttggaagccagcaaagtcagcaagacccagggccatggcctgtgtg
gtatctgtgatggagatgcagccaatgacctcaccctgaaggatggctctgtgctgggtgaggcagaggaccctg
ctccctttctggacagctggcaggtgcccagctccctgacttcagagggtcagacccgcttccgcccagacagtt
gtgccacagctgactgttcgccctgccttcgcatggtgtccaaccgaaccttcagtgcctgccacagctttgtct
ccccagagtcattctgtgagctgtggatccgggacaccaagtacgtgcagcagccctgtgtggcactcaccgtgt
atgtggctatgtgtcacaaattccacgtgtgcatcgagtggcggggctccgactattgccccttcctgtgctcca
gtgactctacctaccaggcctgtgtggcagcttgtgaaccccctgacacatgccaggacggggtactgggccccc
tggacccagagcagtgccaggtgctgggtgagggctgtgtgtgcacggagggcaccattctgcaccgacgccact
cggcactctg
catccctgaggacaagtgtgcctgcactgacagcaccggggttccacgtgctctgggggagacctggaacagctc
cctcagtggctgctgtcagcagcaatgccaggctccagacaccatcatacccgtggacctggactgccctggtcc
ccgacctgagagctgcccccgctttggtgaagtgatcctgttacagcccacggaggacccctgctgcctggggag
tgtctgcgtgtgtaaccagaccctgtgcgagggcctcgctcccacctgccgcccaggccacagcctcatcaccca
cttccaggaggattcctgctgtcccagctacagctgtgagtgtgaccccggtctgtgtgaggctgagcaggtccc
cacctgtcgtgaggaccagatcttgatcgagggccgcctaggagactcctgctgcacctcctacttctgtggctg
tggtgagtgttcagatcccatgcctgagtgtcaggaaggagaggcactcactgtgcacaggaacaccacagagct
ctgctgccctctgtatcagtgtgtgtgtgagaacttccgctgcccacaggtacagtgtggcatgggcacttccct
ggtggaggtgtggagcccagaccgatgctgcccctacaagtcctgcgagtgtgactgtgacactattcctgtgcc
cagatgccatctgtgggaaaagtcgcagctggatgaggagtttatgcacagcgtggagaatgtgtgcggctgcgc
caagtacgagtgcgtgaaagcccctgtatgcctgagccgtgagctgggtgtgatgcagccaggccagactgtggt
agagctctcagcagatggtgtgtgtcatacgtcccgatgcacagacgtgctggaccctctcaccaacttctacca
gatcaacatc
acctctgtcctctgtgacatgcactgcgaggcgaaccaggagtatgaccacccacgggacctggcggcctgctgc
ggctcctgcagaaacgtgtcctgcctcttcaccttccccaatggcaccacttccctgttcttgcctggtgcatcc
tggatcgcggactgtgcccgccatcactgtggcagcacccccctgggtgctgtgcttgtccgctcgcccatcagc
tgccctccattcaatgaaaccgagtgtgccaaggttggtggctctgtggtaccatccttggaaggatgctgcaga
gcatgtaaggaggatggacggtcttgcaagaaggtgaccattcgcatgaccatccgcaagaatgactgcaggagc
aatactcctgtgaacctggtgtcttgcgatgggaggtgcccttctgccagcatctacaaccacaacatcaacacc
tatgcccgcttctgcaagtgctgccgtgaggtgggactgcagcgtcgctctgtgcagctcttctgtgccacgaat
gctacctgggtgccctatacagtacaggagcctacagactgtgcctgccagtggtcctgaagcctgggtgccagg
cctggctgggtcacccttgtcactttcatgttctccttccagctagtctaggtgacaaaggggagaggttaaagg
tgggggatcttgtttgtatgtgcacgtgtgtgtgtgtgtgtgtgtgtgtgtgtgtgtgtgtgtgtgtaatccaca
aggactagaataggccccacttgcccatcctggctctctctctctgagtccttttacatgcacatagaaagtgcc
attgctcagggcttttgggatccaactctacatggcttgaggtcaggagggaagctgtaggcccaggaactgaag
gcctgatcca
tctgtgtctctggccaggacttctagggggaggggccaaggtttcctgatacactgcaaccaaagcagaacccaa
tgctggcttggggttaagatttggtggcctgcttctttcttgtccctcttaacaatggaaaacccataatataag
gaaaggggatttaactaacatttctgactgtctcaagaggtctagaactccttgaagtgaagagggtgagcttgg
ggctcagggatgcccatgaagatctgtgagaccatgctgtgactgagcaggtggctctagactcctcaagcctca
gagcgttccctgacattctggtatctagaagagcaggtccaggtctgtgcctctgtgtgtcacatgctcaccatt
gagccaggtttgagatacatgaaggataggcttctcagtgagggcctgggtgccaatcccagagggtgctggaca
gcccaaccctccaagcagttctggagtacacagctggcacttccagcctaaagccctgtcccttttctccaagca
tgtgaaaagtgtatctgcactaggtcttactgtgttaggtggtggctcagcgctgcctgggactgtgttggtgga
gaatgtctgtaggtgaaaatgcagccctgtgagtttgcaacccaggtggccccgagtgtccgtcagagccttgtc
tccaaaccaaagtgtcttccctaaagccaggatcagagctcctgtccacaagccactttgcagatcccctccttt
ctgcactgctttgtgctactgggtctcaaaggtcatgacctgctgctgttcaggtcccagcactgttctgacggc
cccatggggtaaaggtcctgtgtcgagatggtcccaacatccgctttgtctctgtgactcttctgtatcttcttc
atgtatcaat
aaactgcaagaagttttccaagcaaaaaaaaaaaaaaaaaaa (SEQ ID NO: 22) describes
otogelin (Otog), mRNA. Gene name is Otog otogelin. NCBI reference sequence
is NM_013624.1.
11.
aggtgtcagattgttcatttgaattgttacttattaatgtttgtttgtattctattttcaaacttcctat
taatccactgagtctttttttttaaacatttaaggtttttatcatgaatattttgtttcttctctaatta
taatttttactttattaattaaatatttatttatttgctttctatcctgactatagcttccctcgctcct
ctcctctcagtcctttcccccagcgtgctcccccctccactcctcctctttttctcttcagaagaggcca
ggcctcctgtggatatcagctagccctggctatgaagttgcagtaagactaggcgcatcctttcctgttt
aggctagaggaggcagcccagtagggagaaagggtcccaaaggcaggcaacagggttagagagagcccct
gcttctgctattataagttccacatgaagaccaagctatacaactgaaacatatgtgcagagggcccagg
ccagtcccatgcaggctctctaggtggtgtttccatctctgtgagcccccactggcccaggttagttgat
tctgtaggttttcctgtggtatatttggcacctctgcctcctatgatgcttcctccccctattactttgg
attccccaggctctgcctaatgtttggctgtgcatctctgtgtctgtttccatcagctgctgggggaagc
ctctttgatgatggttaagctaggctcctgcctggaagtgtagcagaatcattagtgtcagtatcaggga
tatataatatcatatataatatataatatcattagtgtcagtatcagggatatgctcctctcctctgtca
tggcctgatatcaagctggaccactccctcaatgtccgctctatctttaccccagcatgtcctgtaggca
ggacaaactgtaggttgaagtttttgtggctgggttggtgcctcagtccctccattgaaagtcttacctg
gttacaggagatggcagatgactctatagcctccattgctaagagtcttagctagggtcacactgggagc
ttccatttccctaggtttctagctcattccagagatgccccccagccctgattccagttgtctctcctgg
tactctctccttccattttcctggcacctgattcttcctatttccatcccaagaccctacactcatccag
cacacccccatctacccacaaggtctattctatttccccttttcagtgggattcaaacatccccacctta
ggccttccttgttacttagcttctttgggtctgaggattgtagcgtggttatcttttactttatggctaa
tatccacttattaataagtacataccatgtttgtctttctgggtctgggttatctctctctggatgattt
ttcccctagttctactcatttgtctgcaaatctcatagtgtcattgtttttaacagctgaataatactcc
attgagtaaatgtagcccattttctttatccactctttggttgagggatatctagattgtttccagtttc
tagctaatatgaataaagctggtatgaacatagttgaacaagtgtcctcacggtatggtagagcatcctt
tgggtatatgctcaggagtggaactgggtcttgagatcaagtcccagttttctgagaaactgccaaattg
atttccaaagtgactgtgcaagttaacactcacaccaacaataaaggagcactccccacaagtttacact
cacacgaagaatggaggggcgctccccacaagtttacactcacacgaagaatggaggagtgctccccaca
agtttacactcacacgaagaatggaggcatgctccccttactccacatccttgccagcatgagctgccac
ttgaggttttgatctcagccattctgacaggtgtaagatggaatctcaaagttgttttgacttttttccc
tgatggccaaggatgttgaacatttctttaagtgtttcttagccattaaagattcctctgtcaaaactta
ctgtttagatttgtatccctttttttaaattggatcatttggtttgttgatgtctttgtacttttgaaat
tatccctaaatgtttgagttttaaaataaacataatgtaatatt (SEQ ID NO: 23) describes
adult male testis cDNA, RIKEN full-length enriched library, clone:
4933407A17 product: unclassifiable, full insert sequence. Gene name is
4933407A17Rik RIKEN cDNA 4933407A17 gene. NCBI reference sequence is
AK016710.1.
12.
ccctcctgtcccctccctagacaaagcaatagggatggttaaagagagtccagcagccctcctccacgct
gtgcccctgtaccgactggaaggatgattcttgccgcgacccttacccttctgtttgcctacaaggatgt
gctgtgcatcacgctgacccagagctccactgaccagacagtggcaagcggcactgaagtaacactgctc
tgcacgtacaatgcggattctccaaacccagatttattttggtatcgcaaaaggccagacagatccttcc
agttcatcctttatagggacgacactagttcccatgatgcagattttgttcaaggtcgattttctgtgaa
gcacagcaaggccaacagaaccttccatctggtgatctctccagtgagccttgaagacagcgctacttat
tactgtgcctcggggtattataccggagggatacgagctaccgacaaa (SEQ ID NO: 24)
describes T-cell receptor delta-chain delta7.3 processed pseudogene,
VDNDJ region. Gene is similar to T-cell receptor alpha chain V region
PHDS58 precursor NCBI reference sequence is M23096.1.
13.
gagattctgacctcttccgtcaacaccatccaactaattcacctgcgtgccaccatgaagccagtgatgc
ttacaaatggaaaccacagcttcttctgtgagcactttggaaagagagagcagaaaatcgggctgccttt
cgggaatcgtgaggccttacacaggatcctagacctccatcgggagctattaaatgggattgaggttaga
ggtggggcaggcattaggggaggatcctgaatttacctctctcctcacaatattttctcagcatcgattg
ccttttctccaggttgtggggaagtaggtggggctcaaaacatattttttttcctactcctgaaatgtag
aacttgtatatgtattttaaaggactactgagcagttctagatggaccaatccaattttctaattcaact
ttataggatgaatgatggaagattactttgactagctctttgtagttctatgaagaaaaccttagcacac
tgggtcagttcttgggtccgctagtagtttagttacctgttctgtggtgcattttcttaatccagtacat
ccaggagtgtgctagcctttctttctctctgtgtttcttagcttggaaacaatcttcctcccttttggca
ctcaaaatggtttcatttacatccagttttatttactcagacaatattgaacttgactatattttgtacc
acatcttctgtttggagctgggaatgaatacacaaggaagtcaatcaaaggcagttctagccttgccctc
accacatgcagtcaaatgtgggctacaacagttaagcaggcttgtgacacgaggaaaagactatgtgaca
cagagctcaagagccttttgggagccctcagggtgtgctgttttccaaccttgcagtgttcggagccagt
agctttcttcagaggcagggatgaagtggagccggcatacactgtggcaatcctcctcctctgaaactct
gcaggataagagaaatcctctcctctctggactttgggatgtggagaggtcttttttctttggaatagtg
tatggcctatctgttccatcgcagcaagagtagcaggttgtcttgggaagacaaaatgtgttactttaca
gggaagcaggagcagggaggaggctgacatttataaagtaactatattagatacaacatttaagttgcct
tccggactttaaatgtacccaactcact (SEQ ID NO: 25) describes 14 days embryo
lung cDNA, RIKEN full-length enriched library, clone: G630026J20
product: unclassifiable, full insert sequence. Gene name is
Gm3364 predicted gene 3364. NCBI reference sequence is AK090251.1.
14.
cttcaagcctgcttcgggctgtgtagccagactgctacaaaggggttaagagagagagagagagagagag
agcaagagagagagagggagagcacgcgcgagagcgagagcgagcattcagctggcttgagctgtgtagt
ctgcagagacttcagaaccacacattccttcttatgtggaattgtgaaaagaacactagatgattttatg
atttacatttctacttttattgcctaatttcttttcacacttggaaggagaagggaatttatccatgttt
ttctacaaactaaaaaaaaaaaaaaaaaaaaacctggcaaaaaatgccatattgcatgttaaaaaaagca
ttaaaatatggattcattaagaaaatcaagtaggcatacagtaatgtctttaaatgattagtatattttg
gtggatttgatctttacaattaaaaatacttaattaaaagtttgattctgttgttgttagactaaaataa
cattttcctaaagagattttaagctgattcaatactgtgaccttttatccttcttttctgtttaatgttc
tcgctggtgaaagtctttgaagactgttgtctaaattctgtatagtgcttagatacatactttgaagagc
aaggagtccattgttgttgattctaaacttgtgttgtatgtctactttgtagacaaaaataaatgcttat
tgacaaaaatttttctatgtaacaaattttatttagcataatcagctccaacaaccagcataaaaagttt
acagagaacagaaagtacattaatgtaagctagtctttacaaaaaggggaaaaatatgttcttgatcaaa
atagtttacacttcagtgataaactgagaatgttttttaaaggggactttatgtgtatgggtactttgcc
tgtatgtacatctgcgcgtcatctacaagcctaatgccaagggaggccagaaagagtatcagatctgctg
gaactggagtgtgagaggctggtgaactgccgtgtggttaccaggcattgaacctgagttctctaggaaa
gctgccagtgcttttagccactgaaccatctctccagcccaaaaaagctttaagaagttggtctctgcct
gacagcagaagtcagtagaggtcactgttccgtgtgttacctatgcattagtgccctcagctggaactgc
ctacaggacaggagacagacacagtcaggaacacatcagctctgtcactctacccgtgaaggcttcttct
gcctagtaaaagatgtgtcttcatagcatgaagttggggataaattcatctttctcagatctattttcca
tcttctccagggcagagcttagttcctcgtctttcttcacaagtttgtatgtcagctacttcttgatcta
aatgagaccgttccttctagtttaactagctgtgacccattttctccatctgccatctttgtcactgact
gccgagagatgctccgatcgtgtcctcacggattgttttgtcacctctgggaccaagagtagtcacagga
gccttgagaagcctttccagcagtgagatcacttcagactaagcagtgatgtgaagactggaggtagtgt
ccatgacacagaactgacacggaagttgtttgctgtggttcctgctctcctggctttcactcagcagtac
tgactttggctcgcccagcagagaactggaactgtgagcacatggtctcactcactttgcaggtgattcg
aagacttaagttggataaaaaacttcgatgaagttataacctttattaaactcaattaataaactt
(SEQ ID NO: 26) describes 0 day neonate thymus cDNA, RIKEN full-length
enriched library, clone: A430108H20 product: RIKEN cDNA 1600023E10
gene, full insert sequence. Gene name is Rnf149 ring finger protein
149. NCBI reference sequence is AK040602.1.
15.
cggactacattgactaagggacaggggcacgatgtactaggctctcaagctcacttagctgtggttatgt
gctccattggtcctctgttggcttcctgggctggtatcagacaacgtagggtaaatctctgattgggagg
ctgctgctgtttgttcgggtctcagagaaccttccctggctaggcaacagaagaagcaagaaatctccac
aggtttcagcgctcatgagctcccaagaccttctacttgattggagctgttgtggacagaacaacagatg
ctgactttggccccaggactctgaaaccatctgactggtgagagccctggatgtacatggatgatgaccg
aaaaatccaacggtgtgaagagctctccagctaataaccataaccatcatcctcctccttctatcaaggc
caatggcaaagatgaccacagggcaggtagcagaccacagtctgtggcagctgatgatgacacttcctca
gaactgcaaaggttggcagagatggatactccacgcaggggaaggggtggcttccgaaggattgtccgcc
tggtggggatcatcagagactgggccaacaaaaatttccgtgaggaggaaccaagacctgactccttcct
agagcgtttccgtgggcctgagctccagactgtgacaacccatcagggggatggcaaaggcgacaaggac
ggcgagggaaagggcaccaaaaagaaatttgaactgtttgttttggacccagctggagactggtattacc
gttggttgtttgtcattgccatgcctgttctttacaactggtgtctgttggtagccagagcctgcttcag
tgatctacagagaaactattttgtggtatggctggtgctggattacttctcagacactgtttatattgca
gacctcatcattcggctgcgcacaggcttcctagaacaagggctcctggtcaaagaccccaagaaattgc
gagacaactatatccacactttgcagttcaaattggatgtggcttctatcatccccactgacctcattta
ttttgctgtgggtatccacagccctgaggtacgctttaatcgtctgttacactttgcccgtatgtttgag
ttctttgaccgcactgagacacgtacaagctaccccaacatcttccgaatcagcaacctggtcctctaca
tcttggtcatcatccactggaatgcttgtatttactatgctatctctaagtccattggctttggggttga
cacctgggtttaccccaacattactgaccctgaatatggctacctggctagggagtacatttactgcctt
tactggtccacactgaccctcaccaccattggagagacaccaccccctgtaaaggatgaggagtacctat
ttgtcatctttgacttcctgattggtgtcctcatctttgccactattgtgggaaatgtgggctccatgat
ctccaacatgaatgccacacgagcagagttccaggccaagattgatgctgtcaaacactacatgcagttc
cgaaaggtcagtaaagacatggaagccaaggtcatcaaatggtttgactacttgtggaccaataagaaga
cagtagatgaacgagaagtcctaaagaacctgcctgcaaagcttagggcagagatagccattaatgttca
tttgtccactctgaagaaagtgcgcatattccaggattgtgaagctggcctgctggtggaactggtactg
aagcttcgtcctcaggtctttagtcctggagattatatttgccgtaagggggacattggcaaggaaatgt
acatcatcaaggagggcaaattggcagtggtagctgatgatggtgtgactcagtatgccttgctgtcagc
tgggagctgctttggtgagattagtatccttaacattaagggtagcaaaatgggcaatcggcgcactgcc
aatatccgtagtctgggctactcagatctcttctgcttgtccaaggatgatcttatggaagctgtgactg
agtatcctgatgccaagaaagtcctggaagaacggggtagggagatcctgatgaaggaaggtctactgga
tgagaatgaagtggcagctagtatggaggtagatgttcaggagaagctggagcagctggagacaaacatg
gagaccttgtacactcgttttgcccgcctgctggctgagtacactggagcccagcagaagctcaaacagc
gcatcacagtgctggagaccaagatgaaacagaaccatgaggatgattacctatcagatgggataaacac
ccctgagccagctgttgctgaatagccataggtgcctgtccagccttgctttgaccccaggagttagaag
tgctgtatagaactttacatttacacacattatgctcatgtccctccgaactcccaaagccttgctgagg
cttaaggttttggctacatcttggagtccccttccaagtccagctaacagccaagcttgtggataatgga
gatcatgtgggttgaatttctaagagcgtgacctcctaagtctcacaaggaatcagagaatagctaaatt
gtccttcctgaggcttttctgggactagataactagacagtgttctctgaagaacactgtgcaccacaat
gcctgcctgactccttttagcttctttatatcgagtcactccctactgcattctgccccaaatacctttt
taatgtgttctctgaacagcctgtttccatgcacttgtacacatttaagaattggctgcagacactgggc
cccctaccctgcctcccaaggcatgcaaggtctgtgaggggagtgggaaggtgggtttgagtgttggtgc
tcagggtcatacttccttgtcagacaatgtcactctgagaaggaggtggctggcagctttggccatcaca
ccttatgcacacaagttctgaagagtttgtgaaggctactgctgcatgagatactgtgaattagaggcac
ttaaaagttaataaattcctttcagctaaaaaaaaaaaaaaaaaaaaaaaaaaaaaaaaaaaa (SEQ
ID NO: 27) describes cyclic nucleotide gated channel alpha 2 (Cnga2),
mRNA. Gene name is Cnga2 cyclic nucleotide gated channel alpha 2 NCBI
reference sequence is NM_007724.2.
16.
agatttctgggaacgacgcgggttctcgcgagggccacccaggtcctgaaggagttaaaagacagcgaag
tctgcaagctgggttagcgttgggacagctcgctcgttagtgtctgtttgcgtcgttagtctagcctgga
gggtaaaagctgtaccggagtctcttctgttgtaatcgtgtttgccaacacccatgtctcccttatttcc
caccctagctgcgaatctccggataaattagatccctcgggaaccataagtgtaaagaacggagcacagt
caaatattgtgaaaagctctttccataaatgtgtgtgtggcgtttttaatgtaggcccgttcttgggcat
gcattttttcccccacggttccactttaggatatatatttaaggtattactccattttagcgaggaagca
atagaaacaaacttggtcttgggggctgcagagatggctaagctgttttctgctcttggagaggacttgg
gttcatttcccagcacctacatggcggcagttccaggggatcccacgtcgcgccaaacaacagaacctta
ctgcctaactgttctgaaatgcagatgttagttggttgagcagggggccctttttgaagccccaaggagt
gagtggacgtcccatgtatcttcagcatctggtgcttcagacgttccttggcttatacctgactcctgcc
cctgcctttcattctcttccgtctttgttgtgagaacacttgtcagtttgttaagatagcttgcaaataa
gcttagagactataattccctgtggacactgtctcttgtgtcgctggtaacacatctttaagtgttacgg
ctctgttaggggaaggcttcagctgctgggaaagtttccctgatgcaggtgttacagctttgctctggct
catagaaaattttccctgtgacagttctgctccaaaaggtatcctggcagtcaggagccaaggaatacca
caaagtcttgctgcacaactaagctcaggcaagggattaatttggagggaaaagctaaggagggtggttg
ccctgcttggaagagaagcaacagagaactgggcagaattgggggatggggaaatatatcaggattctgg
ggtggggggttactttccagggtggtctgggattttattttacttttttcttgtacggcgagacctgggc
tgggaatggctattacagcctttaaaagatgaccattagggaagtcaagggcacatgtgtgtgtgggggg
gggggtctggctatttcctgccttgaagcactagaaaggcagttacagctgttttagcctttacaccctt
gctagagctcctgaggccaagacctagccacttttgtgccttgaggatttacaacggctactaagtatac
aaaatttacaaagggaggtcatggcagaaaagcctgttgcttgagttggtgtaaatggccagcaatacct
atttgaagaaagtggaaaataccacagttgatcctttttagaagtaccatgtgaaggatgtattataact
tcccctcaatatttaatattatacagttacaagaggcttatgaaagcaaattccatagatattccatgaa
ggaacggagttgcagggctctattgtgaccaatcaaggaaaggttataaatatcctgataaagtctggct
atattattttgtttcactttaattttaaaaagatcttcaggttttgtttggttgttgttgttgttgatga
tgatgatgaaaaaaatagacccaagttgtacctctgttgctgggtatgacttagaacacatatattctaa
actgatcagaaaataaaattacaaaaacatatcagaccctccagttaatccagatctgcctgtcaagcat
ctggacaggaaatgttctggaaaccttaacgaaatataataaacttaaaagcatgttaagatgggagcat
gcttgtctcctcagtgagccatgcacttatgaatatccccgaagcttctctcacaaggcagtcgcttaca
gatgctctctgctctgagaccctgctttcggaattacagctgactccaactcttccacctccctttttat
atctccctgctctggataacagcccgatcccaaactggattgccttcactccctggacagttgttttctc
tgaagatccctccccgactggccatgcactaggctacatgttactgatcctgcctactttcttgactaat
aatttgaaaaacttagctttcttcttacccagtgctctcttcctggaagctgtcaagagctctagcttgc
tgaccctttttgttttctcaaacacacacaacatacaccacacacacacacacattgctgttaagatagc
tcagcaagtgaaggtgcttgccccaggcctgaagacagacatgaatttaatccctggagcccaacttatc
ctctgacttccatacatgtaccattgtaaatacacacacaatatgattttttaaaagcacccttaaaaat
caataaaagcaaactcc (SEQ ID NO: 28) describes RIKEN cDNA C030016D13 gene
(C030016D13Rik), mRNA. Gene name is RIKEN cDNA C030016D13 gene. NCBI
reference sequence is NM_175354.2.
17.
ttttttagaggaattatcatgactgctcccaaatatatatggaaagaatatgatcagatgccaaaactgt
gattaatatttaaatattgaataggattcacatccacagcaggatgctttaaggaatggaatgtgttgta
gcacccagagtatttgctaagaggctgaggcagcatgcctgtgtctcagtcttcacaggcccaagtcagg
ccctgatgtcagacagagatgacgtcataggccctagcccactgttgcagagccatctctatttctgctt
tgaattctgatgggcttgccttggagaggtttcatacctcagctaaactttcttttatcaagataaatta
tgggcagtggtcagtactcacttgtcactaagaagatacacaatctaacgagtcctagcacagtctgact
tttttcttagtcatactagctagtcatacactgcaggatctaagttattttatcctgtgccctgtctgct
agtcctcattgttaacagatattcaaagtcagattcacttcattatcatactgtactagaatttgccatc
aaaactttctgctttctgctctttggcactgttctctgttttcatatctagaatgttttacccatcgtta
tcctcatgggtatcaaatcactcacataatttccttgaatatcactttgtgctaagtgtgacttgacaaa
tatatttggatacgattaagagtctgtgaggattactatagatttggcaatgtctagttggagattacag
taccatgtcaaacctccataacaacaaaactagtaaaaagatttggaattaaaacagagaccatctccac
cctagggagttagaaaagccgtgttggatgtcataatctgggctaagtgttcagtaactgtgtagtgctg
gccagatggagcctggtgtgtgagagcagagacgtgatggcaggcagggtcagacattcatggcgaaggg
ctctgcaagctgcaggcatcatttggtctccattcagttctccacttgcactgcatggggataaatctga
cttaaggaagctggcaatgagagccactactggaatgctttcatacacaagctcagccacaaaaggacat
gctgacaaccataacaaataaccgaatgagtgatgaatgaactctgctgagaagcaaagaaataaaaaca
gtttgaggatatgtccaatcatattgtttttcataactgggaactgagtactttttattcatgaagcatg
gtgctggatgctaaggaagacagtactcctcagtctaagaagaaaatcgcatgttaaagagctcttatac
aagtagacatgactgcaaggcttagtaaatgcaagatagataaagtacagggtgccgaggagttggatgg
agcagttttaattaagtgtaagtggagagccagttagcactttcaaggaagagaaagtctgctgagcctg
aaaacagggcacatggtagaaccttctcaggagtgagatgtgaagatgctgaaatggaatagtgctaagc
ttcaaagacccgagaaaacaggaatgaggaataggaaggatagaacacctgatacaggaggagggtctga
ggatggtgggaggtgtttggagggtttagtcaattgaagaggaaattggaagacacgaggaagcatctcg
tgtctgctttctgtgtttttgtagaactaacacagatcattaggaggctgggaactccagttctccttca
gtgcagtcaggagatgcaagtggagtgacgacactgttttctttttcattttttaaagatttatctattt
atttattattagatattttctttatttacatatcaaatgttatcccctttcctagtttcccctctgaaaa
atcccctatcctctcccatcctccccctgctcaccaacccatccactctctcctccattcccctatactg
cggcatagaacatttaaaggaccaaggtcctctccttccattgatgaccgactaggccatcctttgctac
atatgcatctagagccatgagtcccaccatgtgttttctttgattggtggtttagtcccagggagctctg
ggggtactggttagtatatattgttgttcctcctatggggctgcaaacctcttcggctccttgggtactt
tctctagctccttcattggagaccctgtgtactgtctaatagatgactgtgagtatccacttctgtatta
gtcaggcactggcagaacctctcaagggacagctataccaggctcctgtcagcaagctcttgttggcatc
cacagtagtgtctggttttggtggttgtttatgggatggatcccctagtgggacagtctctggatggtca
ttccttcagtctctgctctgaactttgtctctgtaactctgtccacgggtattcttgttcccccttctaa
gaaggatcaaagtatccacactttagtcttccttcttcttgagtttcatgtgttttgcaaattatatctt
gaatagtctaagtttctggaataatatccacttatcagtgagtgcatatcttgtgtgttcttttgtgttt
gggtatcctcactcaggatgataccctccagatccatccatttgcctaagaatttcataaattttaataa
ctgagtagtactccattgtgtaaatgtaccacattttctgtatccattgctctgttgagagacatctgag
ttctttccaacttctggctattataaataatgctgctatgaacatagtgaagcatgtgtccttattacaa
gttggaacatcttctgggtatatgcccagaagaggtattgctggatcttccgagagtattatgtccaatt
ttctgaggaaccaccaaactgatttccagagttgttgtactagcttgcaatcccaccagcaatggagaag
tgttcctctttcaccacatccttgtcagcatctgctgtcaccttagttttggatcttagccattctgact
ggtgtgaggtggaatctcagggttgttttgatttgcaatttcctgatgactaaggatgttgaacattttt
ttaggtgcttttcagccatttggtattcctcagttgaaaattctttgtttagctctgtaccctattttaa
aatatggttatttggttttctggagttttatatatattagatattagtatgaaactattttctacactaa
gaggacaaattagaagaactcatcactttcctttctggatatggcgtcacaaagctttaatcccagcact
gttaaagcagaaactgagagatctctatgaagtcagcctgatctacatagcaagttcccgatagccaagg
ctacacaataagatcttgtctcaaaacaacaagaacaagaatgacagttccatgattcataagagtcttt
tctgaggcttgctttatagtcatttcctctaacataaagcagaccatattggaaggaagctgcttaagct
aagaacctaaacaactcaaaagctttaggaaattcctgaaactgatcaggctcactagacaccaccccct
acttactcacgcttataaaaccagtataaactactgagagatactctcagacaagtagagctgctgagac
gaagctcagattacaggtggtgactgaaagagatgctcaacaatggaaaatagacattttccaagatgcc
aagctgccaacaaatgcactgcaggtttctaattttcataaactcatgcagatgtgggtttttgagatgt
agctatctttgagtcatttttgatcctgtaattaatccttcaatcatactcctgtaagtaatcccaataa
aacacactgcttc (SEQ ID NO: 29) describes 0 day neonate eyeball cDNA,
RIKEN full-length enriched library, clone: E130003N16 product:
unclassifiable, full insert sequence. NCBI reference sequence is
AK053269.1.
18.
tgggcgctttggcagtggcggttccctcgggactgcggggaaggcccaggccgccgccgcctgctcagtt
ctccctcactgcgtctaaggctctcccggcctggctccgcgcccagcccagactacgggagggggaacgt
ggaggcgacggaagagcccgtcgcgcctggggctcccgaaatacaaccagagacctacagagggcagcac
cgagccgtaaacgggtcctccgcactgcaagcttggggtcgccagactgcccaaagccaagtccccctct
ttaggacagggcagggttcgtgcccgaccagtccctggcctggataaaagtcaggaatgtgtctaaccat
cacaagaaccaacagatcctggcggggacttaggactgacctagaacaatcagggttccgcaatccaggt
cccaaaggtgggatcctcaaccgcaggacggagggaatagcctttcgattctgggtggtgcattggaagc
cccaggctctaaaacccccaacctactgactggtggccgagtatgcacccgactgctagctaggcagtgt
cccaagaaccagtagccaaatgtcttggcctcagttttcccggtgacacctggaaagtgaccctgccatt
agtagaggctcaggtcagggccccgcctctcctgggcggcctctgccctagcccgccctgccgctcctcc
tctccgcaggctcgctcccacggtccccgaggtgggcgggtgagcccaggatgacggctgtagaaccccg
gcctgactcgccctcgcccccgcgccgggcctgggcttccctagcccagctcgcacccgggggccgtcgg
agccgccgcgcgcccagctctaccggcctggcgccctccccacgcgggcgtccccgactcccgcgcgcgc
tcaggctcccagttgggaaccaaggagggggaggatgggggggggggtgtgcgccgacccggaaacgcca
tataaggagcaggaaggatcccccgccggaacaggccttatttccccagcgccttatatggagtggccca
atatggccctgccgcttccggctctgggaggaggggcgagcgggggttggggcgggggcaagctgggaac
tccaggcgcctggcccgggaggccactgctgctgttccaatactaggctttccaggagcctgagcgctcg
cgatgccggagcgggtcgcagggtggaggtgcccaccactcttggatgggagggcttcacgtcactccgg
gtcctcccggccggtccttccatattagggcttcctgcttcccatatatggccatgtacgtcacggcgga
ggcgggcccgtgctgttccagacccttgaaatagaggccgattcggggagtcgcgagagatcccagcgcg
cagaacttggggagccgccgcgattcgccgccgccgccgccagcttccgccgccgcaagatcggcccctg
ccccagccctgccccagcctccgcggcagccctgcgtccaccacgggccgcggctaccgccagcctgggg
gcccacctacactccccgcagtgtgcccctgcaccccgcatgtaacccggccaacccccggcgagtgtgc
cctcagtagcttcggccccgggctgcgcccaccacccaacatcagttctccagctcgctggtccgggatg
gcagcggccaaggccgagatgcaattgatgtctccgctgcagatctctgacccgttcggctcctttcctc
actcacccaccatggacaactaccccaaactggaggagatgatgctgctgagcaacggggctccccagtt
cctcggtgctgccggaaccccagagggcagcggcggtaatagcagcagcagcaccagcagcgggggcggt
ggtgggggcggcagcaacagcggcagcagcgccttcaatcctcaaggggagccgagcgaacaaccctatg
agcacctgaccacagagtccttttctgacatcgctctgaataatgagaaggcgatggtggagacgagtta
tcccagccaaacgactcggttgcctcccatcacctatactggccgcttctccctggagcccgcacccaac
agtggcaacactttgtggcctgaaccccttttcagcctagtcagtggcctcgtgagcatgaccaatcctc
cgacctcttcatcctcggcgccttctccagctgcttcatcgtcttcctctgcctcccagagcccgcccct
gagctgtgccgtgccgtccaacgacagcagtcccatctactcggctgcgcccacctttcctactcccaac
actgacatttttcctgagccccaaagccaggcctttcctggctcggcaggcacagccttgcagtacccgc
ctcctgcctaccctgccaccaaaggtggtttccaggttcccatgatccctgactatctgtttccacaaca
acagggagacctgagcctgggcaccccagaccagaagcccttccagggtctggagaaccgtacccagcag
ccttcgctcactccactatccactattaaagccttcgccactcagtcgggctcccaggacttaaaggctc
ttaataccacctaccaatcccagctcatcaaacccagccgcatgcgcaagtaccccaaccggcccagcaa
gacacccccccatgaacgcccatatgcttgccctgtcgagtcctgcgatcgccgcttttctcgctcggat
gagcttacccgccatatccgcatccacacaggccagaagcccttccagtgtcgaatctgcatgcgtaact
tcagtcgtagtgaccaccttaccacccacatccgcacccacacaggcgagaagccttttgcctgtgacat
ttgtgggaggaagtttgccaggagtgatgaacgcaagaggcataccaaaatccatttaagacagaaggac
aagaaagcagacaaaagtgtggtggcctccccggctgcctcttcactctcttcttacccatccccagtgg
ctacctcctacccatcccctgccaccacctcattcccatcccctgtgcccacttcctactcctctcctgg
ctcctccacctacccatctcctgcgcacagtggcttcccgtcgccgtcagtggccaccacctttgcctcc
gttccacctgctttccccacccaggtcagcagcttcccgtctgcgggcgtcagcagctccttcagcacct
caactggtctttcagacatgacagcgaccttttctcccaggacaattgaaatttgctaaagggaataaaa
gaaagcaaagggagaggcaggaaagacataaaagcacaggagggaagagatggccgcaagaggggccacc
tcttaggtcagatggaagatctcagagccaagtccttctactcacgagtagaaggaccgttggccaacag
ccctttcacttaccatccctgcctcccccgtcctgttccccttttgacttcagctgcctgaaacagccat
gtccaagttcttcacctctatccaaaggacttgatttgcatggtattggataaatcatttcagtatcctc
tccatcacatgcctggcccttgctcccttcagcgctagaccatcaagttggcataaagaaaaaaaaatgg
gtttgggccctcagaaccctgccctgcatctttgtacagcatctgtgccatggattttgttttccttggg
gtattcttgatgtgaagataatttgcatactctattgtattatttggagttaaatcctcactttggggga
ggggggagcaaagccaagcaaaccaatgatgatcctctattttgtgatgactctgctgtgacattaggtt
tgaagcattttttttttcaagcagcagtcctaggtattaactggagcatgtgtcagagtgttgttccgtt
aattttgtaaatactgctcgactgtaactctcacatgtgacaaagtatggtttgtttggttgggttttgt
ttttgagaatttttttgcccgtccctttggtttcaaaagtttcacgtcttggtgccttttgtgtgacacg
ccttgccgatggcttgacatgcgcagatgtgagggacacgctcaccttagccttaagggggtaggagtga
tgttttgggggaggctttgagagcaaaaacgaggaagagggctgagctgagctttcggtctccagaatgt
aagaagaaaaaatttaaacaaaaatctgaactctcaaaagtctatttttctaaactgaaaatgtaaattt
atacatctattcaggagttggagtgttgtggttacctactgagtaggctgcagtttttgtatgttatgaa
catgaagttcattattttgtggttttattttactttgtacttgtgtttgcttaaacaaagtaacctgttt
ggcttataaacacattgaatgcgctctattgcccatgggatatgtggtgtgtatccttcagaaaaattaa
aaggaaaaataaag (SEQ ID NO: 30) describes early growth response 1
(Egr1), mRNA. Gene name is Egr1 early growth response 1. NCBI
reference sequence is NM_007913.2.
19.
gagagtgccagtctccattgcaaacagtggcccttgaaagagcaagcttcaaggaaggacaggcaggggg
ggcatcctgaagcctgtcagaaccgtgggccttatccaaagggaaaaagtagctgtcacttgttagagca
gagtcctcggcacagtgtgtccgtcctgtttggaagtgttcccaaaaggactcgtgagctctgtgccatc
aagtacttcccttcatcaagctgaggaggccacagcctccgagaggaactggtgagtgcttatggagcaa
agagcatgtagaaccttctagtctctgaccacgtggtaagatggagcatctgtctcagcaagctttgctc
tctcttttctctctggcctattctggtcccctttctgcaagagtttgattttccctctcttttctctaca
tgtctgcaggtgtgtcagtacatggatggatagacaaacacacacgctcatgcacacatacttaggcaca
tacacacttagatacacacttatacacacacacacacatgctcatgcacacatacttaggcacatacaca
ctcagacacatgcacacactcactcacatacactcagacacacactcacacacacacatgctcatgcata
cattctcaggcacacactcagacacacacacactcagacacacacattcagatacactcagacacacaaa
cacactcaggcacacacacactcacacacacactgagacatgcatgtcccatgtggggataggttgcact
tcctctttcagctgtggcttctgggcagctgctctactctgtcatttctgccttatgaagctttgtccct
attcaagtaacagaaagcagaaagtggcccgggaaactcgaccttcaagcacacagaacaaaagagagag
ccactgtcttcctggaaatggtgcatcaaaactatttgctgggattggcttagagctggcttgatggcta
tggtctatttttggatgggtcaagctggtataaaaaggatgtgtgaacgtgaagatgaagaaatgcactt
tctatggacagaactaagactcacctgaatgagtctaggaaagaaacttaactcagttacaaaatgctgg
ataaagccaagagacttaataaagtactttcttgtcaaaaaaaaaaaaaagaaaagagaaaagaaaggaa
ggaaggaaggaaggaagaaagaaagaaaggagagaaagaaagaaagaaag (SEQ ID NO: 31)
describes hypothetical protein 9130430E04 (9130430E04), mRNA. Gene
name is hypothetical protein 9130430E04. NCBI reference sequence is
NM_183203.1.
20.
gagccgaagccgccgagccgccgcccgcagccgtggtccctacagtcttcatggtccttgacgaggctca
ccactgttcagtgtcctgggttaagaaccacactttcaacctaagtgccgctgagcagctggctataagc
atccccaacgacacagcgggcagacccgagtcctgcctcatgttccggccacctcctgacagtgccagcc
tggaagacatcctgagccaccgcttcaatgagacacaggcctgtgactcaggctgggactatcctgagaa
ccggcctcagtccctaaagaatgagtttgacctggtgtgtgattgaaagaacctgaagaggacctctcag
tcggtgttcatggctggacttcttgttggagccctggtctttgggcctgtctgtgactggtttgtggaca
gtctggtgtactacagcctcagcttccaagtgggggactttggcctggatatctatgtgacacagctgat
atttggagcagtggaaatgcctggccgcttcctcagtgtcctcatgatggagaagctgggtcgcaagtgg
agccagctgtgtactctgactctggctggcatcatgtacatcattatcatcttcatcccaggagatcttc
ccacggtggtcactgtgctggctgtggtggggaagtttgcctcggctgcggccttcaccatctcctatgt
gtacactgctgagctcttccccaccatcatcaggcaaacaggcatggggttggtgagcatgttctccagg
gtcggtggcatcatcacaccccttgtgatgctgctggaacagtaccaccaggcgattcccatggtcatct
ttggcagcctccccattggagcaggcctactgtgcgctcttcttcctgagtcccggggccagaccctgaa
ggacaccctccaggacctggagcaggggctccctgcagggtcccacaaaacagcaccccaaagacaagac
atggaggcttcgggaagaacttccagtgtgagggtggctgttgtgaagagctcgtacttctgactgtggc
tgctgagggccagcctatcaagagcagaggacaggtttctgttttgtcacacaagctgcattcccagacc
tgcctttggggtctttgtaggcattttctcttccatttcttaggggttatctatgtctaggagagatagt
tccattctgtaatgcttaattttcattgtcaccctgaccggatgggaatcgctaaaggaggagcatctct
gggtggtgtctgtcaaggtgttgtcagaagtttaactgaggaggggagactgtccctgaacatgggcagt
atcctattggctaaggtcctgaactaaataaataaaaccatgaagggaaccg (SEQ ID NO: 32)
describes RIKEN cDNA 9330176C04 gene (9330176C04Rik), mRNA. Gene name
is 9330176C04Rik RIKEN cDNA 9330176C04 gene. NCBI reference sequence
is NM_175420.1.
21.
atggccaatctctccacagtgtctgtgttcattctccagggcttctctgctgttcctgcattacagttgctgagc
atggccatctttcttctcatatacctggctgcggttctggggaatgtctctatcatgatagctgtgactcttgac
tcccatctgcacacacccatgtacttcttcatcaaacacctctccctagtggatctctgttctacatccaccacc
ctgcctcgggccctggtggctaccatggcagacaccaaagagatatccctcccagcctgtgcatctcaactcttt
gcttttgtctgctttggctctctggaatgttttctcatcactgccatggcttttgaccgctgtctagccatctac
agacccctgacatatggggtaaccatgagttctcagacctgtgtgtccctggtagtggtggcctgggtcagtgga
ctcctcttttctaccttccacatggtcaacactttctccttgcccttctgtggtcccaacatgattgatcacttc
ttctgtgacatccccccactcatgcatctagcttgtggtgacacccaaggccatgaggctgcaggttttatagtt
agtggatgtgtcatcatgacttgctttgccctcacttgcctgtcttatgttcttattgtatacacagtggttcac
atccgttctgcagctggccgctggaaggccttctccacgtgttcctcccatctggccacagtgcttctgttttat
ggcactggaagttcagcctacatgcagcccacagcccactactccccactgcaagggcgcatggccgccatattc
tattctatcctcatgccaactctgaatccacttatctacagcctcagaaacaaggacatgaaagcagctctgcgg
aagctttatc
cacaggtgccatcctag (SEQ ID NO: 33) describes olfactory receptor 1350
(Olfr1350), mRNA. Gene name is Olfr1350 olfactory receptor 1350. NCBI
reference sequence is NM_146389.1.
22.
gcgcttccctccctccggggctgggcctgtcccggccgtcccggagtctctgtcccgccgcgccgttagctgtct
gtgtcttggccaccgcctccaggcagtccgcagcaagccagcttcttggtccgacgagctcagcgccctctcacc
gcgatgctgtgcttcctcaggggaatggctttcgtccccttcctcctggtgacttggtcgtccgcagccttcatc
atctcctacgtggtcgcggtgctctctgggcacgtcaacccctttctcccctatatcagtgacacaggaacaact
cctccagagagtggtatttttggattcatgataaacttctctgcatttcttggcgcagctacgatgtacacaaga
tacaagatagtggagaagcagaatgagacctgctacttcagcactcccgtttttaacttggtgtccttggcgctt
ggattggtgggatgcatcggaatgggcatcgtagccaacttccaggagttagccgtgcctgtggtccatgatggc
ggtgcgcttctggctttcgtctgcggggtggtgtacacgctcctgcaatcgatcatctcctacaaatcctgtccc
cagtggaacagtctcaccacgtgccatgtcaggatggccatctccgctgtttcgtgcgcagctgtcgtccccatg
attgcctgtgcttcactcatttctataaccaagctggaatggaatccaaaagaaaaggattatatatatcacgtg
gtgagcgccatctgtgagtggaccgtggcttttggttttattttctatttcctaacattcatccaagatttccag
agtgtcactctaaggatatccacagaaatcaatgacgacttttgaaagatcgagaatcctgtctcattcagggag
tgtcgcagac
agtttctggaagtggacagaggacggacgggcttggatgtcaccctgatggggactttatctgtggcacatccgg
gacttgaatttcattaagagttcctagtagttcaatttacaaaggtatgtttccctggaggatggatagcaccaa
cgacactgtagcaatatttttatattttctaaaacaatcttttatgaacaaattcatatgcaaagaagacgaggc
attgcagaaaggggaggattattcttgtatagattctttagactttttatgtaataatgatttatgaaaatacac
taagagaaaaaaatgttaagtttagtactttttattaaagaagccaaatcagggcatattcactttaaaatttca
ttttttaaatacagtgacctgcatacattttcatcacaagagcacttatacaattcaattcatagtgattatata
ccctaatggtatagatttaggtaataaacgaacactttaaagcactctgaattttcagtgcattaaacaaatgct
tttatagtgaaggactcaaaaccatttacagtgcacattaaccagcgaatgtggaagacctcggttcgaaaattt
agccccctcatttacttctccaagggacccacagctttactgccgctgttaatggtgggcccgggaactaattcc
aggtaggatgggctcatcaggccagcttagaaatgatcaaactgcccttctctgtgaccgggcagcacaagttca
attcacttctcagtttccctgtaagccaaagagaatgcagatccaagtcagggagaaaggagagcgctcatagaa
acttccagatgtgggctgctgtttatctgctcctatcaatgcctgtgggccactataagggagtcaggccctttc
cgaagcaagg
cctggagactctacctttcatgcagttcaccaatggcaaagaaacgccagctgttgtggaggaagaagtggataa
caagaccccccagtctgggccagtaggagctgccccaagggcacgttccaaggacagagaccatttggacagcct
tagtgaaagggtacctggcgatgaaggggcacacagcagtgcctcctttggctgctaagtgaagggtgctgctca
gcaggcacttctgccatgacatcctgtgtcttcttcctcagtgtgcagtggtggcaaccaacgtccatttctgag
tcccctgtaacttttcacagtaccgaacatgcccattgtaacactggaacagaaagacagtggctgtcattctga
tgagtgagagaggagtcgatacatttcctgtggaagctggggcggactgaatgcatgctgttctttgctttgagc
gggagccttggttgatgccttcccagaatgcacttggctctcttcgcttccactggagacccgaccacgtgcctt
tacccatagtggaacacagtgccttgtggcatgcaataggtgcttaataaatactcattgaatgtatgcacgcat
aaatggatgaacaagtaacgactagggatgtttgaggtgctaagggttttttttactctagttcacgggtattct
aaggtcaacagctagtctgtgccaattagtaatgttgtctgttttgctttgtgaactattctcgtttccacctgt
tccagctgtgtgagcttcatgattgtgtgacaactctctcctggacagatagcacagaaattgttacatgggcta
aacctgtcttggcaaaccgaggaggcccccaaatcacactctgcagattccatgcgacttctagttttatccctg
ttttggtgtt
atttttaatttctacaaatatgtatttccttggactttgtacccgagaaagtaaaataaaatatttctttatttt
agc (SEQ ID NO: 34) describes RIKEN cDNA 1200002N14 gene (1200002N14Rik),
mRNA. Gene name is RIKEN cDNA 1200002N14 gene. NCBI reference sequence is
NM_027878.1.
23.
tcctcatgtcatctgtgtcttaactgctgagccatctctccagaccagcactgagtttcttcatcaggctcttcc
aagcatctgctctaggcacggagaataaagaactagctccctgcctctagttctggatgttagctccagttccaa
gacagccaactgaaggaaggatcctaagtattgcaactcgagtgaggtctgggcacgatgtcatggacctcgaat
cccagtactaggaaggtgaaggtaggaagatcatgactatgaggccaacctgggctacatagagagttccagact
aacctgatctatatagtgaaaccctgtctcatagaatgaaacaacatgaaactggacatagcaattaagaacact
tgttgctcttccaggggacctggggtcaatgcccagcactcatggtaacttgcaaccacctgtaactccagttca
gagcatctgacaccctcttctgacctccaacacaggcatgcacatggtgcacatacagacagacaaaacatccat
tcacataaaatttttttttttttttggtttttcgagacagggtttctctgtatagtcctggctgtcctggaactc
actctgtagaccaggctggcctctaattcagaaatctgcctgcttctgcctcccgagtgctgggattaaaggggt
gcgccaccaccacctggcaaaaataaatctttaaaaaatgtaaaaataaacaagagccccaagagccagatgtag
gaagtggaagctggcacaaggaagtgcccagctgggcatgggttagaactgtgaccaacaagaataacaagacag
aggcagctttagccaacctactgggacagcagcagtcatggaggtgccaggcttcattctgaccagcttggtgac
ataccagtgt
ctgtgtttatctggtggataggtgtctgagactccttaccattctttcttggcaatttttgattgaatgcactag
gattctcaggctagcccttaatgcagtgtgcttattacagctgtggaacatgtgtgcacagacccaggagagcat
agggagatacgagccattgtgacagtctggtgcacgacaagatgtcagcctcaccttccatttaaacagttggtg
agagactttcatagtatgtaactgagtcggggctcgcctcattttaactggaagaagccgccagagcactgtata
gaatccttctcaacgatatgggaatgcaaggtgcaaaggaattcttgctgcactgtgggatgtggattagcttac
agagtgcttgcctggcatgcaagaagccctgggtttaattcccatactaaataagctgatgatagtggtgcatac
ctattatcctagcactcagtaggcagaggcaggaaaatgagaagttcaagattgcctttgattacatagtgaatt
caaggaagtctgggccacatgagaccttacctttaaaagatataagtaaaacatgagttggaagccaacctggac
taaatagtgatcaccaggtcaactagagtcacgcaacaagaccctgtctcaaacagactaacaagaaactaaaat
ggctgatgaggtagcttagtgggtaaaggtgtgtgctgccaagtctgacaacctgagtttgatcccagaagccgc
acagggaaggagagacaactctcgactctggaacgttttcctttccttcagattaatgccatggtgtgtccacaa
atgtatatgcaaatacgcattaagtgtaaataaacaaaaccctgaaagc (SEQ ID NO: 35) describes
0 day neonate head cDNA, RIKEN full-length enriched library,
clone: 4833417J20 product: unclassifiable, full insert sequence. Gene name
is RIKEN cDNA 4833417J20 gene. NCBI reference sequence is AK014716.1.
24.
atggaagtctgtaactccaccttgagaagtggcttcatcttgatgggtattttggatgacaatgattttccagaa
ctgctctgtgctactatcacagccctgtaccttttagctttgacaagcaatggactactgctgctggtcatcacc
atggatacccggctccacgtgcccatgtatcttctgctctggcaactctctctcatggacctgctcctcacatca
gtcatcactccaaaggccatactagattatcttctcaaagacaacactatctcatttgggggatgtgcccttcag
atgttcctggctttgacacttggtactgcagaggacctccttctgtcctttatggcctatgacaggtatgtggcc
atttgtcatcctctgaactacacaattctaatgagtcagaaagtctgctgtctcatgatagccacttcatggagt
cttgcatctctcagtgctctaggatatagcatgtataccatgcaatatcctttctgcaagtccaggcagatcagg
catctcttctgtgagattcctcccttgctaaagctggcctgtgcagacacctccacatatgaactcatggtttat
ctgatgggtgtgaccttgctctttccagctcttgctgccatcctggcttcctactcactaattctattcactgtg
ctgcacatgccctctaatgagggcaggaggaaagcccttgtcacctgctcttcccatctgactgtggttgggatg
tggtatggaggtgctattgtcatgtatgtattacccagttccttccacagccccaaacaagacaatatcagctca
gttttttatacaattttcactccggctttgaatcccctcatctacagcctaaggaataaggaggtcactggagct
ttgaggagag
tccttgggaaaaggttgtcagtacagtctacattctag (SEQ ID NO: 36) describes olfactory
receptor Olfr704 (Olfr704) gene, complete cds. Gene name is olfactory
receptor 704. NCBI reference sequence is AY317825.1_CDS_1.
25.
acttaacttgactctcaagccatgtctgcttatgaagttcactgccacagtaagcaatagactcattcaaaatgg
cgggcgtctggtcttcacttctgcctgcaaatgacaagattcttttaaactgtttttctggaggttaacaaatgc
ccttctgcaggttggtagttttaggctattccagtaaaacacacagtaacaacatagaaactgaattttatcaac
tagatttactttatggagtgaaaaacaatctgacatagcaagcaattgaaaatctcatgtgtgtatgtgcatctg
tgtgtgcctgtgtgtgtgtatgggtgtatctgtgtgtgcctgtgtatatgtatatctgtgtgtatctctgtgtgt
ctgtgtatattgtgtctttgtacatatgtgtgtgagtgtctgtaccttaaaaaataatcccattctgaaaatgca
ttcttagtaacacttatttccagtaacgtatgggaagtgttttctagggaaatatagaggaagtgacgacaagat
cacttatattcctcaagtacttgagaatgttagctatagaacaagacttagtacattgtcaagcatccagattct
gaaagatgtgttagatataaatgagatttgagagagcagggagggaggaaaggaaggagggaggaagggagggag
aatatgcaagtagctgtctggtattgtgtaggcacattcctggtcagatggtggctaggtgcagtatcactgaga
tattaaaaatctacaatgtgatctctgccttcaggatatctatccccatgaattgaataaattgctctcaaacac
ttagcttttaatttgcaatttatctcacagtcgaaactaattttaaaattaaccttctagtcccaagaatattct
ttcacactct
ctacccttgtaaagggacaccgattgtcacttgtttaaaaatggagagacttggatctctttgctcatagttcat
caactgtattctatccgccgccacagagttatttgttataattctttactcttcaataatgggtaattagtgcag
aaagacctagtgcggtttgatgtggaagttaaaagcctcaagaggattccttaaccatgcacctgcctcagctgg
cttatcccagctcagtttggggtttaatgaaaaacatatttttgttgttatagctgagtctcctattcttgacgc
cacccctcattaagtggggttcccctagagtgccacttggaaaggtatctgttgttaggcgagaggcaggggact
taactaatggctgaagcatgcattctacaacatcataggacaggcatggtgaatagttttacacacacacttggg
gactactaaatagaggagttgatacagttttgtggaatttgcaatttttataaaacatgtaaataactatataaa
tatgttaccaaggttcttcctaagcattggttacaatgaaacttaaatgtgcctaatactaagatctttccatct
ctgactcatggggtaatgcaaaattgtttttaaaatagtaacttttcatttgaaaggtactttatatcaggctca
aatttagtacctatttatatgctgagcaataggggtgggcacatacatcaggagctatgaagtgttatacttcgc
ttgcctgctgcttgaagaatctgaagaaagcatatgaaagtaggacctgacttgggaaggtgtgtcacagactac
agagtcagccttttgggaagttttcagtgtcttcagtattaaaaaaacttgtatgtcttactgccatttgctgtt
gatcagtagc
agtgatttgaatacactattgcacagacgtctgatggctgggatgggaaccatacttaaaatccaatgggcagag
ccagctctgtcaccctagctttgttggtcttgaaagagatctctaaagttgagtgtcagttaacagactgttaac
tgttagttgtgattgcttcttcatccacaagaatagagtagctgtttggttgatcatatttccattcttaaaatg
tacactgaaatagtttaagatgtattgattaaacatagcatatttatgactagagttttc (SEQ ID NO:
37) describes 16 days neonate thymus cDNA, RIKEN full-length enriched
library, clone: A130023E07 product: unclassifiable, full insert sequence.
NCBI reference sequence is AK037520.1.
26.
gagagagagagagaagagagagagagagagagagagagagagagagagagaaagagaaacccagggacctcctaa
taggccccatgtcttcaaatcctgcctctttaaacatgtgaaccattatcactagggtacactcaagtgctagcc
actccatactacatccctgttctggagatacccacaagtttttacagatcgggaaactgaggctctgagaggtgg
ggtgagaggtatagagcttatggcacagtcaggatcagaaagtgggatgtcacctgaaagcagccacttggagca
aaggcagaggaccctgggagctccctggatagggtccatacacacacagacacacacacacacagacacacacac
acacacacacacacacacacacacacacacacacggagacaggggaggggggaggcatcaatgtggagaagggac
ctgtggccagccttggagaggcagtggctggtgagtgtgtgtgggtgtgagagggtggaaagggtatatcataga
gcaatctctatgaaagtgtcgtgtggggtgagtgaccgttctggacacccctctaatatagacttttgagcagag
gagtgcctggcccaggagtacctgggagtggggcttgagggaatgaagctggctggtaatctccacctctggctg
gagcctgcaagtccttggaggccccagagcctcatgttctcagagtggggccagggagggaggggcttctcggca
gctggcccaggggccatggcggaaaaacaagccgtgc (SEQ ID NO: 38) describes 16 days
neonate thymus cDNA, RIKEN full-length enriched library, clone: A130027B06
product: unclassifiable, full insert sequence. NCBI reference sequence is
AK037572.1.
27.
tgatcagtataggttgtgttcaaataccagtcgttttattttattctttttgaggtagtaagtattaaattcagt
cacacgccggcccttttaaagagttttattaataagctggagagatgcccacagttaaagaccactggctgctct
tccagaggacagttttcagtactcacaagggatcaccaccatctttaactccagttccatgagatctgatgcctt
ctgctctccagaggcactgggcaggtacatggtatacatacatgcatacatgtaggcaaaacacttatacatata
aaattatttaaaaagatttaagtatagggtttcatgcagtccaggctgacctcaaactctctctgtcatctgatc
ttcctgcctcctctcctcaggttctaggagactggcatgctccactaggcttgatttttttttttttaatgctgt
gctttcgaatgacctcagagcttggtccatgctaggcgagcactgtgccccctgagccacatctcccagtctctc
taggaggggggattttttttttaatagaagggatttgagcatacgtagatttggtatgagaggtgtccctgaaat
cagtccccttgagggtatagaaggacatcatatatcagggatgtccacacttgttgcttttacaccagacaaaac
tgggcactgaattaacacgtctccatttgttcagccacacttccagcacctgcctctgtcagctggcaggtgtga
ggaggtagcccggaagcagagcccgtggaaagatcttttaaaattaatgaacataaatacatctggaccaaaaca
ccgtttaggcacctttgatgttgagatctaaagcgcccgtctcaaattgtcaaacttttgagagctgacataact
ctgtgactgg
aacattccacgctcgaactcctgtgtgacaggtggtaaaatgtggctgtagcaaaatattgtatgaaattacctt
cagaccatgtgtatgaggaatatacatgaaaaaaaaaacataaatgagttgaatgtttaaacctggctacgattc
cctagctagcttgctgtgtaagtgaaatatttcaaaatgataataataataaaaagaatcccacaaatgtaaaac
atttgtggtccaagcattttgtaaaacctctgctaaacctgtatgactttggtaaaaactgtaaacaaaacaaaa
caaaacaaaacaagacagaacaaaacaaacaaaaccaaaaaccaaaaacaaccaaaaacatcaaaaaataaaaca
aacc (SEQ ID NO: 39) describes adult male medulla oblongata cDNA, RIKEN
full-length enriched library, clone: 6330442C03 product: unclassifiable,
full insert sequence. Gene name is A430057L12Rik RIKEN cDNA A430057L12
gene. NCBI reference sequence is AK078093.1.
28.
gacacaattctaagaggagatgttggcagagctggcgtcttctaagccctctgtccttggcagatcgatatcttc
ttccagcattcccctgccatgtcattcatatggccaactcatctgctccagattctgtagcatgcccaaggtgaa
acaaactagataaaacctgtactccggacaatggaagctccctcatcttgcgccacacaaaatagccactcccca
ccgtctggtgatgtctggaatctgtgtgaagtctcacaagagataggaaatctaatctaggaaggaattacaggc
acctgccacagaacaactcaggggtaagaatgtgcttccggcacggtttggtcaagaagctcacagctgtggagc
actgcctgggcttctcgtccattccttcctctctcccctcctcacagtgttggctgggtgtgcccacttggagct
gcgatggctgctgagtcccaacactccacttcttcaccaccctggaggagcctgaagcagctttggagctctgag
gtagacaagcacaccttctcagaggtgcttggacatcctatgcaggaagctctgaagagtcttccagcccgaggc
tactgtctaccttttcctccaagattgaaacatttcatcttgcagagaaggggcttacgcgaatggtcaactcag
aatggtttcaggaagtccacgaccctaaccaggttcttgtagctccttcttgccaagtcaagctgctgagagccc
ctctcagacaggacaagccgaaaagaagaccagaccagctgcctggaaggagcagacacccgctgaccggcctgg
aagaggtttagcccaactgagtcacctgaagagagtactcatcatcctgctgagctgcctgtcagccatgcagtc
cttccagatt
cccagctctgtgagctgtcacccaggctgcggtgggcttcggtgaggcagctgtctttgagtcacttctgttcct
gtcatggatggtcctgactacccaacttctcaagcatccaatgtcctcagagaaagggagaccagagcctgaaac
ctgcttgctcaggggtggagcttcccgatcaatcgttctgccacgcccactgctggaacctgctgctttgctgct
tggagccatactcgtggtcaccctgctactggactctgagattatttggtgggaatcgggccccttcccctgctt
cataactgcgtgcagaacagtaaatttgagctttgatc (SEQ ID NO: 40) describes adult male
diencephalon cDNA, RIKEN full-length enriched library, clone: 9330198N18
product: unclassifiable, full insert sequence. Gene name is RIKEN cDNA
9330198N18 gene. NCBI reference sequence is AK020395.1.
29.
ggacgccccaggacacgactgctttcttcaccacacctctgacaggacaggacagggaggaggggtagaggacag
ccggtgtgcattccaacccacagaacctttgtcattgtactgttggggttccggagtggcagaaagttaagacga
ctctcacggcttgggttgaggctggacccaggaactgggatatggagcttctatcgccgccactccgggacatag
acttgacaggccccgacggctctctctgctcctttgagacagcagacgacttctatgatgatccgtgtttcgact
caccagacctgcgcttttttgaggacctggacccgcgcctggtgcacgtgggagccctcctgaaaccggaggagc
acgcacacttctctactgcggtgcacccaggcccaggcgctcgtgaggatgagcatgtgcgcgcgcccagcgggc
accaccaggcgggtcgctgcttgctgtgggcctgcaaggcgtgcaagcgcaagaccaccaacgctgatcgccgca
aggccgccaccatgcgcgagcgccgccgcctgagcaaagtgaatgaggccttcgagacgctcaagcgctgcacgt
ccagcaacccgaaccagcggctacccaaggtggagatcctgcgcaacgccatccgctacatcgaaggtctgcagg
ctctgctgcgcgaccaggacgccgcgccccctggcgccgctgccttctacgcacctggaccgctgcccccaggcc
gtggcagcgagcactacagtggcgactcagacgcgtccagcccgcgctccaactgctctgatggcatgatggatt
acagcggccccccaagcggcccccggcggcagaatggctacgacaccgcctactacagtgaggcggtgcgcgagt
ccaggccagg
gaagagtgcggctgtgtcgagcctcgactgcctgtccagcatagtggagcgcatctccacagacagccccgctgc
gcctgcgctgcttttggcagatgcaccaccagagtcgcctccgggtccgccagagggggcatccctaagcgacac
agaacagggaacccagaccccgtctcccgacgccgcccctcagtgtcctgcaggctcaaaccccaatgcgattta
tcaggtgctttgagagatcgactgcagcagcagagggcgcaccaccgtaggcactcctggggatggtgcccctgg
ttcttcacgcccaaaagatgaagcttaaatgacactcttcccaactgtcctttcgaagccgttcttccagaggga
agggaagagcagaagtctgtcctagatccagccccaaagaaaggacatagtcctttttgttgttgttgttgtagt
ccttcagttgtttgtttgttttttcatgcggctcacagcgaaggccacttgcactctggctgcacctcactgggc
cagagctgatccttgagtggccaggcgctcttcctttcctcatagcacaggggtgagccttgcacacctaagccc
tgccctccacatccttttgtttgtcactttctggagccctcctggcacccacttttccccacagcttgcggaggc
cactcaggtctcaggtgtaacaggtgtaaccataccccactctcccccttcccgcggttcaggaccacttatttt
tttatataagacttttgtaatctattcgtgtaaataagagttgcttggccagagcgggagccccttgggctatat
ttatctcccaggcatgctgtgtagtgcaacaaaaactttgtatgttttattcctcaagcgggcgagtcaggtgtt
ggaaataaag
acgctaatttata (SEQ ID NO: 41) describes myogenic differentiation 1 (Myod1),
mRNA. Gene name is Myod1 myogenic differentiation 1. NCBI reference
sequence is NM_010866.1.
30.
atgcctcgaggtcaaaagagtaagctaaatgccagggagagacgccaccgtgccagaaattatgctcaggcttct
gaaggtgctgaggaaacagtagtggcaacagaagagtcctctcctaaatcatgtggcacctctgagatgccagaa
aaaaaatcagtttccacagtaactgttgatctcttttcgatatcagatgacagttttattgatcttgaggatgct
gaagacctttttagggattaccacactattagtcaccaagatattctgacccaaaaggtctttgtgctggtgcag
atcctcctcaaaaactacaaaacaaagcagcttactacaatagaagatatgatgcaggttattgatgaggaggaa
atggatgacttccccgagatcctcaggaaagctgctgaaagacttgcagatgtctttgcagttgaactgagggaa
gtcgaatcaagccggcgagtgtatgatcttatcagtaagctgaaacttcccaacaatgggagggttcgtgctggc
cagggcttcccaaagactggtttcctgatgactgtacttggcataatcttcatgaatggcaactgtgccggtgaa
gaagacatctggagaacactaagatctatgggtatatattctggaaagaagcaccaaatctttggagagccccgg
aagctcatcactcaaaatttcgtgaagctaaagtatgtggagtgccggcaggtagccaacagcaagcctccacga
tatgagttcctatggggtcccaaagcatatgctgaaaccaacaagatggccatcctaaagtttgtggccaaggtc
aatgaaatctcccctagttacttcaaagatctttataaagaggctttaaaagatgatcaagggaagaatcagggc
aatcataatg
tcagctctgccattcatctcaaagatagggcattttcatttgtgatgtgtccacggcttgctttctccaatgttg
aagtttaa (SEQ ID NO: 42) describes gene model 44, (NCBI) (Gm44), mRNA. Gene
name is gene model 44. NCBI reference sequence is XM_907821.
31.
atagggagacttttctcaagagaattaaatacccaccgcatcagcccagtgggagccaggatgcatggccttttc
cttatgccagaaattatttggggagccacccaagctcagggcaatctgctgggctccccaatgcctgaagccagt
ggcagggtgcttcctgtctgccctggtgatggcaggggagtgagactggctattcacactcattacagttagtgc
agtgggcagggtgtcactgactatgtaatcccaaggtctccaaacctcttgggaacagactaagcatccataggc
tagaggactggaaaactgggccaattgtccagttaccatggtgatgaggggcctctcacctcccagccccacagg
ggtgtggccttcagaggccagagtagctctgaaatagaatagagacagagattattcagaaggtagaaagtggga
cagggagagagacttgcagcaaaagttaccactgatagttattctcttgtctgtgatcctgtcattcgggatgct
gaggcaggaggattacacgtgcaagaactgtatagagaaaccctgtctcagtaaaggaaagagcagccataaact
gagcccatgctgactgcccgcctccttgccctccactccctcccctaccactcctctttcctctcctcctcccac
cctcttccctcttggttcccctcccgcttccctctgcaccttatccccacatgcatgcacccacccccgtctgca
gggtaggcgacgctgtcctcggactggcctccatgttgctgctgcttgtgctgaagtgtatgcgggaacacatgc
ctcctccccatcctgagatgccccttgccgtgaagttcagccgtgggctggtgtggactgtcacaacagctcgca
atgccctggt
ggtctcctccgcggctctgattgcttacgccttcgaggtgacaggatcccatccctttgttctgactggaaagat
cgccgaggggctccctccggtgcggatcccacccttctcagtgaccagggacaataagaccatctcgttctctga
gatggtgcaggacatgggggccggactggctgtggtacctctgatggggctcctggagagcattgccgtggccaa
atccttcgcgtctcagaataactaccgcattgatgctaaccaggaactactggccattggcctcaccaatgtgct
gggctccctcgtctcctcttacccagtcactggcagctttgggcggacagctgtgaatgcccagacaggggtgtg
taccccggcaggaggcctggtgactggtgccctggtgctgctgtccctgaactacttgacctcactcttctccta
tatccccaagtctgccctggctgccgtgatcatcacggctgtgaccccactctttgatgtcaagatcttcaggag
tctctggcgcgttcagaggctggatctgctaccactgtgtgtgacgttcctgctgtccttctgggagatccagta
cggtatcctggccggtagcctggtgtctttgctcattctcctgcactcggtagctaggcccaagactcaggtgtc
agaaggacaaatttttgttcttcagccggccagcggcctgtacttccctgcaattgatgccctccgagaggcaat
aacgaaccgggcactggaagcatccccaccacgttccgcggttctggagtgcacgcatatcagcagtgtagacta
caccgtgatcgtgggactcggtgagctcctggaggacttccagaagaaaggagtcgccctggcctttgttggcct
acaggtgccc
gtgctccgcacactgttggccgctgacctcaaggggttccgttacttcaccactctggaggaggctgagaaattc
ctgcagcaggaaccaggaactgagcccaacagcatccatgaagatgctgttccagagcaaaggagctccctgctc
aagtctccctccggcccctgaagagcagatggtataggaagggtttctggaaggttctgtcaccatgacttggag
tcacctgatagactcaccaacctggtgggacttaaaaggcactgcataggtggctctggggaacagcagggagcc
atgtatgatttccagggtgtcactttcctgctgtcccctaggtgtgagtatttgagggctgggctgactgaaaag
tctttcagagagagagagagagagacagagacccagagacacacacatggcttctggcctggtctggcagggtaa
ggtgacactctccagatcccagattcttctttggaatcaggtcctactggagaaaaatcaaagagattgggcatc
tcggagatgtgtctgaccatgtcatgaagtctaatctgagctgaagaggtggccacagcatgccacacaaggtca
attctgtttaaaatcatgtgttttttaaatggaagtcactctggtgtttttgtaaagcaaaacaaaaacatttat
gcttactttaaaaaaaaatccaagaactcatgcatcctgttcactattctcttaactgaggtcccctgcccggac
aggtgtgtatccgtcagtgtgtgcacacggtgccgtctgatgtgaagacccgaatcactgtattaaggtgctttg
taaactgtcttcatgcagtcattaattgtgaattatttattgtgatgatcaatgacattaaatgcaagatttatt
taccttatta
aaatacaagatttatttattg (SEQ ID NO: 43) describes solute carrier family 26,
member 11 (Slc26a11), mRNA. Gene name is Slc26a11 solute carrier family 26,
member 11. NCBI reference sequence is NM_178743.2.
32.
agcgattgattaatagctgggcgaggggacacactgactgttataataacactacaccagcaactcctggctccc
caacagccggatcacaggcaggagagagtcagtgacggatagactttttttttctttaagaagccaacaacttgg
ttgctagttttatttctgttaatttttttcttttttttggtgtgtgtggatgtgttgtggtggtcttttctaagt
gtggagggcaaaaggagataccatcctaggctcagttcaacccctctccaaaaaaaacggcttctctggcgctcc
agctgcctgacagcctctacccggtggaagacctcgccgcctcgtcggtgaccatcttccccaatggtgaactgg
gaggcccctttgaccagatgaacggagtggcgggagatggcatgatcaacattgacatgactggagagaagagac
ccctggatctcccgtatccgagtagcttcgctcccatctctgcacctagaaaccagaccttcacctacatgggca
aattctccattgacccacagtaccctggtgccagctgctatccagaaggtatcatcaatattgtgagtgcgggca
tcttgcaaggggtcacccctccagcttcaaccacagcctcctccagcgtcacctccgcctcccccaacccactgg
ccacgggacccctgggtgtgtgtaccatgtcccagactcagcctgaactggaccacctctactctccgccaccac
ctcctcctccttattcgggctgtacaggagatctctaccaggatccttcagcattcttatcgccgccatccacca
cttccacctcctctctggcctaccagccacctccttcctacccatcccccaagccagctatggacccaggtctca
ttcctatgat
cccagactatcctggattttttccatctccgtgccagagagatccacacggtgctgctggcccagatcgaaagcc
gtttccctgtcctctggactccctgcgagtgccccctccactcacgccactctctaccatccgtaattttactct
ggggggtcccggtgctggagtcacgggaccaggagcaagtggaggtggtgagggacctcggctgcctggcagtgg
gtctgcagcagtgactgccaccccttataatccgcaccacctgccattgcggcccatcctgcgacctcgaaagta
ccctaacaggcccagcaaaacgccagtgcacgaaaggccctatccctgcccagcagaaggttgtgataggaggtt
ctcacgctctgatgagctgaccaggcacatccgaatccacacgggccacaagcccttccagtgtcggatctgcat
gcgaaacttcagccgaagtgaccaccttactactcacatccgaacccacaccggggagaagccctttgcctgtga
ctattgtggccgcaagtttgccaggagtgacgaaaggaagcgccacaccaagatccaccttcggcagaaggaacg
gaagagcagtgctccctctgcacctccatctgcccagtcttcagcctctggtcctgggggctcgcaggccggggg
cagcctgtgcggtaacagcgccattggaggaccactggcctcctgcacctctcgaaccaggacaccgtgagatga
agctctggctgacacaccagtttctccaggccccagaggccctctgtccgagctgccaacactaccacccttccc
tgttcctccccatgatcccgtgatctgggcaaaggaccttgatggagcccagctctgtcccaccttctcacggac
ggccttccga
aaacttaggccatttgaagggagttgactgtcactccaagaaatgggggagcaaaaagagggctgggtgagggcc
cctggcctacagggctgcgctctgaccctgactgagagatgtctgactatggtctgctagccctttccgttgacc
ctggatgccagttgttctgagactttttctacaataggttgggagttgctgattcctttgatcgaggacagcgga
aaagactaaattaaagcaaaactgatgtggcactttaatggcttgggactgacttgggggtgggggtggggagtt
gtacagtgagcagagcctcatcctggctgctgcactctggccctagagcagtgaatggaggtttctctggccatc
tcaacccttaagcaatatgtcctagaaactcaagagaatggaagtgcaatgtcgggcaggacaaagcaatattgg
ctcctttttttgttgtagttgaggaacaaagattattttttcagtgtatatccatttagattttgtgtatttttg
atgtgcactgctctccgagttctgaaccttcgggaaaaaaatgtaaagcatttatgatctcttgaaatgagtcaa
aggttaactaacttatttaaagggggacgtacataggatgcatgcagtgagtggtgttgcaagtgtcctctgtgc
cttgtgtgatgtgggcagtgttaacagggtctgcatgtgtacaggatgccttactatgggaacagaaaaatcact
ccttgggtttaagtatggctgtatatttctgcctattaatatttggaatttttttagaaagtatatttttgtatg
ctctgttttgtgacttaaaagtgttacctttgtagccaaatttcaggtgagaatgtgcttcaatgtcactgccgc
tgatttgttt
ggttattagctcttaatagttgtggagagagaaacaatctattctaacataaaaaaaccactaactggagttcag
ataatggatggcttattgactatggtgtaaataaatacttttcaacaataaaaaaaa (SEQ ID NO: 44)
describes early growth response 2 (Egr2), mRNA. Gene name is Egr2 early
growth response 2. NCBI reference sequence is NM_010118.1.
33.
gagtctaccatgtgggagaagttctaaaccgccacataggttgcggttacccctcctaagtaagttgaactttga
aaacgtataattactcaccaaggctaagctgaccccatttctcaaccgactgagcctcttggaggaacgtttatt
acgtaatcactacccgtggaagaacagagaccgatctgagcctcggagggctcagacatcaggacatcagcatcc
caagtggcttcgttcctttgaaaaaattgctgtagcacttagcagtctgcttaaaggagggagagaatcacactt
tgatctctgataagcgcaaattcaagtccttgagctcagcctttcaacatcactggcggttggcgtggtctccat
cacagtggcggccattccgaactgttgtcgctgcagctctctccctttgatcccatcctgccaggtctcggagtt
cgcctgccatggctctacttactgcggcagcctggttcttgggaaccaaaaacccaccctgccttgtccttgccg
cccggcatgccagtgcttcttccacgaacttgaaagacgtgctgaggaatctgatacccaaggagcaagccagaa
ttaagaccttcagaaagaaacacgggaagacagttgtgggccagatcactgtggacatgatgtacggtggcatga
gaggcatgaagggacttgtgtatgagacatcggttcttgatcctgatgagggcatccgtttccgaggctacagta
tccctgagtgccagaaactgctgcctaaggctaagggtgggaaagaacccctgcctgagggcttattttggctgc
tggtaactggacagatgcccacagaggaacaggtatcttggctctcacaagaatgggtaaaaagggcagctctac
cttctcatgt
cgtcaccatgctggacaattttccgaccaaattgcaccccatgtctcagctcagtgcagccatcacagtcctcaa
caatgaaagcaactttgcccgggcatatgcgcagggaatgaaccgaactaagtactgggagctcacctatgaaga
ctgtatggacctgcttgccaagcttccgtgtgttgcagcaaagatctaccggaatctgtaccgggaagataggaa
tattgaggccattgactctaagctggactggtcccacaattttaccaacatgttaggctacacggaccctcagtt
caccgagctcatgcgtttgtacctcaccatccacagtgaccatgagggtggtaatgtaagtgcccacaccagcca
tttggtgggcagtgccctttctgatccttacctgtcctttgcagcagccttgaatgggctggcggggcctctcca
tgggctagcaaatcaggaggtgcttgtctggctgacacagctacagaaggaagttggcgaagatgcatcagatga
gaagttaaaaaactacatctggaacacactcaactcaggacgggttgtcccaggctatggccatgcagtactgag
gaagactgaccctcgctattcctgtcagcgagagtttgctctgaaacatctgcctaaggatcccatgttcaagct
ggttggtcagctgtacaagattgtacccgatatcctcttagagcaagggaaggttaagaacccttggcccaacgt
agacgctcacagtggggtgctgctccagtactacggcatgagggagatgaattactacacagtcctgttcggagt
gtcgcgggcactgggcgttctgtcccagctcatctggagcagagccctaggcttccctctggaaaggcccaagtc
catgagcacg
gatgctctgatgaagtttgtgaactctgagtcaggataaaatgggagacggggggtggggggggacgactgccca
taagaaaacgaggataaaaatttattaaaaataaattctaatttttgttttgttccagggc (SEQ ID
NO: 45) describes citrate synthase like (Csl), mRNA. Gene name is Csl
citrate synthase like. NCBI reference sequence is NM_027945.1.
34.
cagggagcgtcgcaagtttcccagccgcgtgcgaggacgcggtgcgggccagccagctagccccgcgtgcagcgc
tcaggaccgggctgactgcctaggagcatgggcagcgaccggagcgcactcggacgtccaggctgcactggcagc
tgcctcagcagccgagcttcgctgctcccgctgctactggtgcttctggactgtctgggccatggtacagcgtct
aaagacgccgaagtgtacgccgcggagaactggctgcggctctatggctacctaccccagcccagccgccacatg
tccaccatgcgctctgcccagatcctggcctccgcccttgccgagatgcagagtttctatgggatccctgtcacg
ggtgtgcttgatgaagagacgaaaacgtggatgaagcggccccgatgtggggttcctgatcagtttggggtacat
gtgaaagccaacctgcgtcgacggcggaaacgttacaccctgacaggaaaggcatggaacaattaccacctgacc
ttcagcatccagaactacactgagaagctgggctggtacaactccatggaggcggtgcgcagggctttccaagtg
tgggagcaggtcacaccattggtcttccaggaagtatcctatgatgacattcggctacgaaggcgagcggaggct
gacatcatggtactctttgcctctggcttccatggcgacagctcaccgtttgatggcgtgggtggctttctggcc
cacgcttatttccccggccctggtctgggtggggacacccatttcgacgcagatgaaccctggaccttctccagc
actgacctgcatggaatcagcctctttctggtggccgtgcatgagctgggccatgccctggggctagaacactca
agtaacccca
gcgctattatggcacccttctaccagtggatggataccgacaacttccagctgcccgaagatgaccttcggggca
tccagcagctgtatggctccccagatggtaagccacagcccacccggcctctccccactgtgaggccccggcggc
caggacggccagatcaccagccacctcggcctccccagccaccacatccaggtgggaagccagaaaggcccccca
aaccagggcccccaccccagccccgagccacagagaggcctgaccagtatggccccaacatctgcgatggcaact
tcgacacagtggctgtgctccgcggagagatgtttgtgttcaagggccgctggttctggcgagtcaggcacaacc
gcgttctagacaactaccccatgccaattggccacttctggcgcggtctgccggggaacatcagtgctgcctacg
agcgccaggatggacattttgtcttcttcaaaggtaaccgctactggcttttccgagaagccaatctggagcccg
gctacccacagccgctgagcagctatggcacagacatcccctatgaccgcattgacacagccatctggtgggagc
ccacaggtcacaccttcttctttcaagcagacaggtactggcgcttcaacgaggagacacagcatggagaccctg
gctaccccaagcccatcagtgtctggcaggggatccccacctctcccaaaggggccttcctcagcaacgatgcag
cctacacctacttctacaagggcaccaagtactggaaattcaacaacgaacgcctacggatggaacccggccacc
ccaaatccatcctgcgggacttcatgggctgccaggagcacgtggagccccgatcgcgatggcccgatgtggctc
gtccaccctt
caaccccaacgggggtgccgagcctgaggcagatggtgacagcaaggaagagaacgcgggtgacaaggatgaggg
cagccgcgtggtggtgcagatggaggaggtggtccggacagtgaacgtggtgatggtgttggtccccttgctgct
gttgctctgtattctgggcctggcctttgctctggtgcagatgcagcgcaagggcgcccctcgcatgctgctcta
ctgcaagcgctcactgcaggagtgggtgtgatcaggtagcacccgcagcacccggtgctcaggtctgcagggaca
ggagacgtgtggatcccagatgctccctcggcgcggcaggatgccctcctagccacacacatccccctgccagcc
ttgccccccacccccgtcctcctttatttatgcccaggtgcctcttctttttggcaccctctctcagcctttggt
tccgctttcctgactcgggccaggagatgctttgagatctcccggagtgtagtaccgccccaggcaggaacgaag
ggacagaggctcacacctctggaagatggcagagggctgggaagcttctctggctctggcttttcagcctgaacc
tctcctcccacgacaagaactgtgtttgccttgaaggagcccacctcgggctctgccctcctgaatttgccttag
ggagagtatatgctcccccccctggggggggcaccctgataaccttgagacttggttccttcttcttaggctcct
aagaagcccagaagcctaaaagccctcccccatcctagcattctgcagctcctacttgccccgggcctgcagctt
ctttcctctaggaaggggccctggacatgtctcacctaggaccaaagggagcctgccaagctcccctcccaatgc
tagtgactag
ctcctccttccctaggctgcccatattctctttcagtgccccataggtctccggaggctgggcactccccgcccg
cccccacccccagcttctgcagtccttgtcccaactcttaatgacatggggcagacacacccactagggtaccag
aggtccctgggcagtgtgtgtgattgtggatgctcagcaggtcacagaccaagcttatgggcttcctcccttcca
gattcctgtccccacacaacactactaggctcaggccagcagctcaccccacacagagtctgtggaacccccaaa
ggaatccactaaggcctgggaagtctgtcctaagggtgaaaggggccctcccttcctcttctcatcctggagtca
gcaggaggcttctgtgggaaatggtactgtacagctgtggctctgtctcctctgcctccctaaagccttgagcag
taaagggctgccagccaggatcctctgggtcaccgccgctttccaagtggtggattcttatagcctggcactaga
ggtgacagatcggaggggcatgtcctgcagcactctagatgtcccctctgcagaacctgagagtcagcaaggggt
ggggcgtccacccccccactccccatcgtttagttctgtagataatcagcactgattaaatgtccagccagcgag
cgtggctgcacttgtacgctcagacccaagcatgccgcaaggaagacctgcttggcagggtcggggccaagcgag
gatgccgaggaataccctcctcccagtttggtaggacctttgggatggagtccaggaaggtcagggaagagtggg
gggggggggcagtaggagtctggttgggctgaggtttactgggtttacttaacatttggtagtgataagaggaga
ggacagccca
gcttcccaaatgactccacatctggcggtctggaagaaccagacaaggccgcccaatcgtcccacccacctggat
tggattttgggcagcctgagaggtggcttgaacaagaactcagggatgggtctggagagaattgtgtaacggggc
cactttacctccagaatcttccctccatgttcagagaataaaatgcactggaacctgcaaaaaaaaaaaaaaaaa
aaaaaaaaaaaaaaaaaaaaaaaaaaaaaaaaaaaaaaaaaaaa (SEQ ID NO: 46) describes
matrix metalloproteinase 15 (Mmp15), mRNA. Gene name is Mmp15 matrix
metallopeptidase 15. NCBI reference sequence is NM_008609.2.
35.
gcggcgcccccactttccaacccctgcggcgagagcggatgcagatggtccgggagacaggcctctctttctgat
gaaaagaggagggaaggatggactacactcaccaacctgccctaatcccatgcggacaagacaagtatatgccca
agagtgaacttctcctacatctgaagacctacaacttgtactatgaaggtcaaaacctgcaactccgacaccgag
aggaggaagacgagttcattgtggaggggttactgaacatctcctgggggctgcgcaggcccattcggctgcaga
tgcaggatgaccacgaacgcatccgtcctcctccatcctcctcctcctggcactccggctgtaacctgggcgcac
agggcaccacactgaagcccctcaccatgcccactgttcagatctcagaagtggatatgccagttgaaggcctcg
agacacacagcccaacagattccagaggcctgaagcctgtgcaggaggacaccccacagttgatgcgcacacgca
gcgatgttggggtgcgtcgccgtggcaacgtgaggacatccagtgaccagcgacggatcagacgccatcgcttct
ccatcaatggccatttctataaccacaagacatcagtgttcacaccagcctatggctctgtcaccaacgtccgca
tcaacagcaccatgaccactccgcaggtcctgaagttgctgctcaacaagtttaagattgagaactcggcagagg
agtttgcgctgtacgtggtccacaccagcggtgaaaagcagaggctgaagagctccgactaccctctgatcgccc
gcatcctccagggcccttgtgaacagatctccaaagttttcctgatggagaaggaccaggtggaggaggtcacct
atgacgtggc
ccagtatataaagttcgagatgccagtccttaaaagcttcattcagaaactccaggaggaggaagaccgggaggt
ggagaagctgatgcgcaagtacactgtgctccggctaatgattcggcaaaggctcgaggagatagcagagacccc
cgagactatctgagccacaggaatgaagacagtccagacactccaggggccgaggctgacctgagaagacccact
ggaagctaggcatcctacagaaacgttttcacacgatccttgcagccttccaaaagccagcactgctccctacag
taggacacagaagcagcagacagctccccacctttggacccctgatcttctttgtctcattctcaaggacttttg
tcctaaggaggacccacaatgtgtatgcttgcatacctgagtgtgtgtatgtgtatgtgcacgcatacgcaagtg
tgcatatgtgtacttaccatgtccacgtgcctctctgatgttcttacatttggttaaattcccagcagccagcag
gtgtcacaaggcaggagggcgcacgtgctgctcctgtgtgggaagaacagggaagtgactgaggcttttggcttc
ctccagtgccaacggcagccgcacggatctgccacgcttgacagggaagaggaggcagcaggctcaccgtttgga
atgtctgaaggtttacagcagagattcttctcatttgccaaggggtggccccatgcccttggggcaagtccaacg
aaatggaaaaagcaagatcacctcagggcggatgcaatgaatctatgagtttttgcactggaaaggttatcaatg
tagaaagcacaattaatgtccccgtcctggatggaaacctagaaagaatggattctgccttcgttcttggctttg
tggaggcttc
tgggtgtttctgctctccagctgccacttcaattcaccctgacctgctcatgtgcctgggccaggtgtgtgctgg
gataaacggcactcgaaagtttttaagccaccagctgagatgagaggtacctttaagcctagcccaccatcacat
cggtgagagtatctcccacgtggaggtggagcagtgtgtttgaagtcgcatgtctttctttctggtctctgcaca
cttgcaagctccttcacctgatacacggagatggattccaagaatggagacctgtcctgttgctcagtcaacaca
gggagtcaggaagctctttggggtgttccatgagggtgggcagagaggacggtgcttgcttagcctttgagaagc
catgctcccgattttcagaaccagcctcattggcagaaagggcagggatgatcctgtaacccctccattcgggat
gctgtcatgtagctggtcacctgtgtggcttgtaccatttgagtttggacggatggatgggcttgctgctggtct
atgaaactcagccaagcaaggggagcagcccataactcaagcatctctgagttttgctacaacagggtgttcttg
tctacctgctctcaagaaagctctagaagcacagcagcggagagactgaggggccccgaccagtgcgccttgtaa
ggaaggtggacctctgcagcagatctgttctagtgtaggaatgttactgtcctttgcccttctcagaggctcaca
gctgtgatatcattgtgggctttcaaacagatggctcatggccagccattttgctgggtatcacttagcaacctt
gctcttcttctaggacagcacaatccatcccatgagcagtgggatggaaacctgtgggtctgagtctttatctta
gactcaaaag
acagcaaagcacagaactgagcacagatactgagtgtatctttatgagtacacacttcagtaaactctgagctca
gtgggcagctggggtgggggttcctttatgacagacaaagaacagctttgtgaatgcaaatacttgagaaaattt
ggtttggataacttctcttcccgttatctttatcttgaatgttcagagcctatgttggtccacaattctcccttg
ggagcctgggggctttccatggagcacacgaactgggaagttatttctgatttatgaaagctcattgtgtaagtg
tcaatatgtgactgttgctgggcatcgggttgccaggctttccaagatggtggtgccaagatagacagcttttaa
agactggggttcagaaagggagttggggtacatgttcactaatccatcttggcacattccctagagatcatgcct
tatgtccaaagaactgggcaggagttttaggaaattggctgggaatgagactcagtggtggaacattttcttagt
gtgtgtgaaaagccgacttcaatctctagaaacacacacacacacacacacacacacacacacacacacacacac
acacacacacacatgcacgcatgcacgcacgcacgcagcagtggtgtagctcagtgatagagggattgcttagag
tgtcagaggctggttcccgacgcctgcaccaagcaccacactaccccctgcactttggaagcctttgacatagcc
gcagcagaacagacctctacagtgacgactggattttctcactggtctcacttcacagctttgaactccaagact
gattagtgtgggcttatgtaattgggtgcaatggacctgaaatcctgagaaataaaagactaacgcataaggcgg
cggtgggggt
gctaatccagcatgtgaaaagtggttcggctgcctctggactcttcccctcccccacccccacccccacaaaggc
ttattttacaaggtgatcaagtgccgagaacaccagcctccgcctgagcgaggtggtgaggccaagcacaggttg
actttctcggtgaccatgttagcggggagctgatgcctcacagtgggtgggggtgttagttggaaaggggcccaa
agcacaaacacccgtcatgcctgtgctactggtgaagggcacacagctaacaccatggacaaacgcagctttgta
acgtggaaagtttggggttagggatacatttccaagaaagaaatatgtccagtgaccaaaggagttttaatgtcc
cacaggagtcagctttgccctggtgagggaggaggtaggaggctgccctagctcacccccacagcactgcctatc
ttaaagcatatcacctaggttgttttctggaaagcctggcttccgccggatgagagagcatctatgaacctgatg
tgacatcccttccttcagacataattgacagtggttgccattccttggggtacaccttcctcccttcctcctgct
cccattaaactgagcacagaggtgcttgaactgtgaactttggagaagcctcagtctccaggttgctggattctg
gggttgctgggttctggggttactactagtgtttcttcgggttttttcttagttgagtggagtcttttttttttt
tttttcttcatcataacttgcctgttaggaatggtacatgacttggcttaactgttggttatgaactgaaaatcg
gatgtgcttaggaatcttaacatgattggatgggaccttgatggggagccatggttttgtagccttttatttttt
aattattatt
ttttttgtaatgtatccatcttagaccttatttaaataaagcacttttggaagcgaaaaaaaaaaaaaaaaaaaa
aaaaaaaaaaaaaaaaaaaaa (SEQ ID NO: 47) describes Ras association (RalGDS/AF-
6) domain family member 2 (Rassf2), mRNA. Gene name is Ras association
(RalGDS/AF-6) domain family 2. NCBI reference sequence is NM_175445.3
36.
cgacgttgtgcttctaacagctctccgaggtccctgccggaagtgtaggaagaagcagacagatttgaacatctc
tgtttccagcttctctgatcatcatgatgaagattagacacaaaaacaaaaaaccaggtaaaggttccaaaggct
gtaagaagcctgcaaggcaaaatgggaagaaagtaacctccagaccatcatctgctccccagattgttcatggca
atgaccatgccagtcgcgaggccgaattaaagaagaaaagggtcgaggagatgagggagaagcagcaagttgccc
gggagcaagagagacagagacacaggaccatggagagctattgtcaggatgtcctgaaacgtcagcaggaatttg
aacaaaaggaggaagttttgcaggaattaaacatgtttcctcagttggatgatgaggccacaaggaaggcctatt
acaaggaattccggaaggtggtagagtactctgatgtgattctggaagtcctagatgccagagacccattgggct
gccgctgtttccagatggaggagactgtccttcgtgcagaaggcaacaagaagctggtcttggtcttaaataaga
tagatctcgttcccaaggagattgtggaaaagtggctggaataccttctcaatgaactgccaactgtggctttca
aggccagcacccagcatcatcaggtcaaaaacttgactcgttgtaaagttccagtggaccaggcctctgagtcgc
ttttgaaaagcagagcctgctttggagccgaaaatctcatgagggtcctggggaactattgtcgcctgggggaag
tgcgtggccacattcgtgtgggtgttgtaggccttcccaatgtggggaagagcagtctgatcaatagcctgaagc
gcagccgtgc
ttgtagtgtgggagctgttcctggtgtcacaaaattcatgcaggaggtctacctagacaagtttatcaggcttct
ggatgcaccaggcattgtcccaggacccaattcagaggtgggcaccatcctgcgtaattgcatccatgtgcagaa
gctggcagaccctgtgaccccggtggagaccatccttcagcgctgcaacctggaggagatttccagctactatgg
tgtatctggattccagacgactgagcactttctgactgcagtggcccatcgcttgggaaagaagaagaagggagg
tgtatatagtcaggaacaggctgccaaagctgtgctggctgactgggtgagtgggaagatcagcttctatacact
accaccgcccactcacactctgcccacccatctcagtgctgagattgttaaggagatgactgaggtctttgatat
agaagatactgagcacgccaatgaagacaccatggaatgcttagctgtgggagaatccgatgagctgttgggtga
catggacccacaagaaatggaggtcaggtggctccattctccactggtgaaaatagcagatgctattgaaaatag
aagcaccgtgtataagattggaaatctcactgggtattgtaccaaaccaaaccgtaatcagatggggtggcctaa
acgcaatgtggaccaccattgcccccaaaataaccgtgtagtagaggtcagttctgtggaccgccgcccgatgtt
gcagaggatcctggagacagacccacttcagcaaggccaggctctggcatctgccttgaagaataagaaaaaatt
gcagaagcgttcagataaaatcgccactaagttgtctgactccatgatgtccatgcttgacctctctggcaactc
cgatgactgt
gcaggtgactgagcagctgaccttccccctcatactgcaagtactgcttcccgtgcgatgggagagtccagatgc
ctttccattctccctgatgtacttaataggagaacacctccatgtcatgtatcttctgctgccgtgccctccatt
cttatcaaaggtctggagctgccaaaccctactaccctctacttaatccttcctgtctttgaaaatgaccttgtg
tttgttttgtggtgccagggattgaacataataggttctcacaaatgttagacaaccattctattaccaagctgt
agtattaccatcattaccatccatcattatcattggggagtctgcttactcaacctcccaagtactgggattaca
ggcttttgccaccattcccagctcccactgtgttttctaaggtaaataaaatcagcagactcttggcatggtggc
aacatatctataaccctgacacttgggagactggagctaggaggactgtaagttatgggccagcctgaactatct
ggcaagaccctttctcaacaaaacagcccaaaacaaataagcagagtccagctgggcatacacctttaatcctag
cactctggaggcagaggcagatgaatctcgtgagtttcaggccagcctggtctaccgagcaagttccaggatagc
cagggctgcatagagaaactccgtctcgaaaagtgaacaataaaacaacaaaaagaatttgtaattatttaaaaa
gcaaataagcagagtccaagtgctgacaatgtaccttggactctaaattcctgtgtcgtaccctgtgagttaggc
tgataagggttaaccttttggacaggtaaatgtcctaaaggctagactgctgagaaacctggtagcagtggcggg
ggaggtggcg
gttgtccctggatgggtgatccttttcatttttatcaccctacaagatctcagtgccaaggctcttgcctgtgta
ttttccaaatcaatagctagctcctcatctgggtagtgtaggagtagagaaggatagtgtgagtctcctgtctct
ggaagtgtctggggatagtgtagaggatgacgattctagaaagggtatacctatcatagtaagttggaaagccat
tggctgcaggatgttcaggaaatgtagaccgtacctgttttctggacggttaggggctgagcacaaactactaaa
caatgagaaacagttaccacctttaactttcactatatccgtatcaggaaggggtaaggctcagaaagccgacag
aatgtgctcatgtcaacagtaagtggtggtgtgctagatgtcaaacttgagtctaatttaaaccttatgctagcc
ttttggtttgcattattttgccttttcaacaagggcacaccactctggtattttctgttgtcatatatttaaaat
atattttctctactctgttttttatttttatttatttatttattttttgggtttttcgagacagggtttctctgt
atagtcttggctgtcctggaactcactttgtagaccaggctggcctcgaactcagaaatccgcctacctctgcct
cccaagtgctgggattaaaggcgtgcgccaccaccgcccggctctgttttttaaaaaatggtagtatttattgaa
aaactatgctttctaagtctctttaaatgatgtccaacttaattcactgatctcatcttaagttttgctagacaa
taatatattagaaataaaagtatctagattcttgggaacataatcatgtcaaaataaaatagaactaaaaccagg
tgggtggctc
acaccagtgctccaagcactaagaatgctgaagcaggaagatgtctgaagctgagtccagcctgggctgcagagt
aagaggcagtctcagggcgaaacaaaataatgtgggactttagatattctaactctaatgtatctcaaggttggg
ggtagcatgcgcacgcacgcacagagagagagtttcccccatataacccagtgtggtcctgaactttttaggccc
cccccccattagctttccaagtgctgggattgcaggtgtttgctttcatgcctggatatttctttgttcttaaaa
caatttcttgggtctttactctccccttaaaaatcatttttatacaataaatctcatccttcatggttctatggt
tctgtaagtttagacacctttttttttttttttttttttccgagacagggtttctctgtgtaatcctggctgtcc
tggaactcaccaggctggcctcgaactcagaaatccgcctgcctctgcctcccaagtgctgggattaaaggcgtg
cgccaccacgcccggttatttagacaccttttaacaggggtggcctgccatcataatcaagataagagaattttg
tggcatttccccaaacttattgtgtcctcttgtagtatagtcctgcccccatcctcaaatcctagcaggcacaga
cctctttcctgttccttaatataagtgtcgtaagtcacacgggggcttattttatggcattaagcttttagcttt
ttccttttagatcttacattgaagttagatgcagttgtataagctcggataaatggataaagccgagtaatcatt
aatacaggacagaagagttctattagcctctcctttaccattcctggtaatttatagtcaaaccttgttctctcc
ctgacagtca
cggacttgtgctttcactttatggcttgtcttttgaaaagagttgtaagtggaatttaatagtatgtaacttttg
agattaatgtgttataaattatagacttgagatccatatcactggatgtatcaatagtttgatttctgtttcata
tgctattgtgtggatgtcttattgcttattcaaccctaagttggattacattctttacagttgaggaggaagtta
taaataaagctgctaaaaatcaaaaaaaaaaaaaaaaaaaaaaaaaaaaaaaaaaaaaaaaaaaaaa (SEQ ID
NO: 48) describes guanine nucleotide binding protein-like 3 (nucleolar)-
like (Gnl3l), mRNA. Gene name is Gnl3l guanine nucleotide binding protein-
like 3 (nucleolar)-like. NCBI reference sequence is NM_198110.1.
37.
ccacgcgtccgctcctttaccaagatggcgtcttgttcctgtttcgccacagttcctaccttatgagcttggatt
cctcacgctaataaaagaggagcagtaaaagctggcaggctgacagaggcaacctcaggacggactttctggcta
ctgaccattttgctgtgtcttactaggatcgtgtgtggacgtgagatcaaccatgagctccgttgcagttttgac
ccaagagagttttgctgaacatcgaagtgggctggttccacaacaaatcaaagttgccaccttaaattcagaaga
ggaaaatgaccctccaacctacaaggatgccttccctccacttcctgagaaagcagcttgcttggagagtgccca
agaacctgccggagcctggagtaacaagatccggcccatcaaggcctctgtcatcactcaggtgtttcatgtacc
cctggaagagagaaaatataaggacatgaaccagtttggagagggtgaacaagcaaaaatctgccttgagatcat
gcagaggactggtgctcacctggaactctctctggctaaagatcaaggtctctccatcatggtctcagggaagct
ggatgccgtcatgaaagcccgcaaagacattgttgctagactacagacacaggcctcagcaactgttcccattcc
taaagagcaccatcgctttgttattggcaaaaacggagagaaactgcaagacttagaactaaaaactgcaaccaa
aatccagatcccacgcccagatgaccccagtaatcagatcaagatcactggcaccaaagaaggcatcgagaaagc
tcgccatgaagtcctcctaatctctgctgagcaggacaagcgtgctgtagagaggctagaagtggagaaggcttt
ccaccccttc
atcgctgggccgtacaacagacttgtgggtgaaatcatgcaggagacagggacgcgaatcaacatccctccaccc
agtgttaaccgtacagaaatcgtcttcactggagagaaggagcagctggctcaggctgtggctcgcattaagaag
atttatgaggaaaagaagaagaagactacaaccattgcggtagaggtgaagaagtctcagcacaagtatgttatt
gggccaaagggcaattcactgcaagaaatcctggaaagaactggagtttctgttgagatcccaccctccgacagc
atctctgagactgtgatacttagaggcgaacctgaaaagttgggacaggcgttgactgaagtctatgccaaggcc
aacagttttacagtctcctcagtctctgccccttcctggcttcatcgtttcatcattggtaagaaagggcagaac
ctggccaaaatcactcagcaaatgccaaaggttcacattgagttcacagaaggtgaggacaagatcaccctggag
ggccccacagaggatgtgaatgtagcccaagaacagattgaaggaatggtcaaagacctgattaataggatggac
tatgtagagatcaacattgaccataaattccacagacacctcattggaaagagcggagccaacatcaacagaatc
aaagaccagtacaaagtgtctgtgcgcatcccccctgacagtgaaaagagtaacctgatccgaattgagggagac
ccacagggtgttcagcaggccaagcgggagctgctggagcttgcttctcgcatggaaaatgagcgcaccaaggat
ctaatcatcgagcaaagatttcatcgcacaatcattgggcagaaaggtgaacggattcgagaaattcgtgacaaa
ttcccagagg
ttatcatcaactttccagacccagcacaaaaaagtgatattgtacaactcagaggccccaagaatgaggtggaga
agtgcactaagtatatgcagaagatggtcgcagacctggtggaaaatagctattcaatttctgtaccaatcttca
aacagtttcacaagaatatcatcgggaaaggaggcgcaaacattaaaaagatccgtgaagagagcaacaccaaga
ttgaccttcctgcagagaacagcaactctgagaccattattatcactggcaagcgagccaactgtgaagctgccc
gcagccggattctgtctatccagaaagatctggccaacatagctgaagtggaggtctcaattcctgccaaactac
acaactctctcattggcacaaagggccgtctgatccgctccatcatggaggaatgtggtggcgtccatattcact
ttcctgtggaaggttctggaagcgacactgttgtcatcaggggtccttcctcagatgtagaaaaggccaagaagc
agctcctgcacctggctgaggagaagcaaaccaagagttttactgtggacatccgagccaagccagaataccaca
aattcctcattggcaaagggggaggtaaaatccgaaaggtgcgtgacagtactggagcccgcatcatatttccgg
ctgctgaagataaggaccaggacctgatcacaatcataggaaaggaggatgctgtccgtgaggcccaaaaggagc
tggaagcattgatccagaacctggagaatgtggttgaagactatatgctggtggaccccaaacaccaccgccatt
ttgtcatccgaagaggacaagttttacgagaaattgctgaagagtatggcggggtgatggtcagcttcccacgct
ctggcacgca
gagtgataaagtcactctcaagggtgccaaggactgtgtggaggcagccaagaaacgcattcaggaaatcattga
ggatctggaagctcaagtgacagtggaatgtgctataccccagaagtttcaccgatcggtcatgggtcccaaagg
ttcaagaatccagcagatcactcgggactacaatgttcaaatcaagttcccagatagagaggaaaatccagttca
cagtgtagagccttccatccaggagaatggagatgaggctggagagggcagagaggccaaggagaccgaccctgg
ctctcccaggaggtgtgacatcataatcatctctggccggaaagagaaatgcgaggctgccaaggaagccctgga
ggcactggttccagtcaccatcgaggtagaggtaccctttgatcttcaccgctacatcattgggcaaaaaggaag
tggaatccgcaagatgatggatgagtttgaggtgaacatccatgtaccagcccccgagctgcagtcagacaccat
cgccatcactggccttgcagcaaatttggacagggctaaggcggggctactggatcgagtaaaggagctgcaggc
tgagcaggaagatcgggctttaaggagttttaagctgagtgtcactgtagaccccaaataccatcccaaaattat
cgggagaaaaggagcagtgatcacccagatccgcttggaacatgaagtgaacatccaatttcctgataaggatga
tgggaaccagccccaggaccaaatcaccatcacagggtatgagaagaacactgaagctgcccgtgatgctatcct
gaaaattgtgggtgagcttgagcagatggtttctgaggatgttccactggatcaccgtgtgcacgctcgaatcat
tggtgcccgg
ggcaaagccatccggaaaataatggatgaattcaaggtggacattcgcttcccacagagtggagcgccagacccc
aattgcgtcactgtgacaggactcccagagaatgtggaggaagctatcgaccatatcctcaacctagaagaggaa
tatctggcagatgtggtggacagtgaggcactgcaagtttacatgaagcccccagcacatgaagagtccagggca
ccatccaaaggctttgtggtgcgagatgctccctggacctccaacagcagtgagaaggctcctgatatgagcagc
tctgaagaatttcccagctttggggctcaagtggcccccaagaccctcccttggggccccaaacgataatgatca
aaaagaaacctttccagcctgctgcccaaacccaaccacacaatggtttgtctcaatctgacccagcagctggac
cctcgtaaattgttgatgctctttcccctccccgaggtctcgcagcaaagcctggtgggctggccttgtgtggct
gcttctccaggcctggtcagcacccagcaggctcagggtctgctcctgatgctgtgctctgggacaggcacgcca
ctgtgtgaaacactaagcaaggtaatcgagcatttcgtgatcacagactccagcttcctggtccacccagcatgt
agtcagcactctgaccttcacaccagagctccacagcggctaggagttgacttcctgtgtcatgacctcaggaaa
taaatttccttgactttataaaagccaaaacgtttgccctcttcctttcccacctccctcctgcccaccacctgt
cgcccagaccttcctctgttgaaagcagtcagcttcctttagctgtcaaaatctgcttagcacaggcttcaggat
aggaggaagg
catggtatcggacagcaagaggtcagatgtcctggtgatagagtgggctgagaagtagagggtggcctgggtcct
cagatagtgctgaccgtgcccactttgggttccgaaagcatcaggggcttctattgcagctttagcctcatgatc
ctatgtatcttagaggcacagctgtgtgtattccacttgggttccctgtgactatcacagtggactagggtggct
tgagactttgggggaaaggggaggtagagactggcttttcttttggtgatggggagggggctcaaccctaagtga
cagctcagctttcccacgacctttagcctctgcctgccttgttcagttgccttctttctacctcagccagtgtgg
tagtactctcatgtgggttagtgccacccaaggctcttcttgtgatgtagacatttctggtgattctgggcttgc
cattgagttgagggtggaggccagtgctttccctcagtccacatgattcagggactgggccagagctgggtgttc
cttcccagccagcctgaccatgcaggctctggcaagcataacctggatgctttatgcagtcctaaggaacaggct
taaggggcaggtctcgtcgatagaggtggtcatttagggtggattcacagggaaggcagaatgaaaacaacccaa
agcagggagcacagctatctgaaggagatcttttcctcaggtacaagaggtaatttttggaccactggcctccag
ctagcttggctgtaggccaaccctcaccaagaaaagtggtcccgagcagacaaacccctgtcaggctgtggaaac
tgaggacagaccttttatctccaggtcatctgatcaactagcaacatgtcccctcccagatacttgggttactag
aaaagccaac
ttgtggcgatctcatggccttccctgggaactgtcacttctactatgatacctttccccctcaagtctttgactc
ccccatttaataactcaggcttggctacctgtagactctaatcctaagtggatccagaattgctttcccaagtct
aacttcagttccctttggatggaaccagtcccacatgtacccacttagaagaacgatactgagatgggaaaagaa
catctactttgttcagtaggctggcagcttaagccaggtgggtgagacggggagagcgcttccctgcctttccct
ggagccgtctcttctcccatctgcatgagactggggacaccttgcttccctccttcccttccagccccctcttca
tcttcattgtcatcctacaacatccaatctcccctcactgaaggtaaagtttaaattatcaccaggattttgcac
atctccttttattgctaagtgctgtagatgtaagccctatcctaagctgttcagggtctcactccttttctctga
ttgttaataaggactatgaaattgactgtacccaggtcacctctctgcagcctccaagatgttgaggagcctagg
gcaggggtgggagtgtcttttcctccttgttgaatgtcttccatgtcctatgtttccatagctacatccatccct
tggccttaactttggaatttggagattatatgcaaacatgtgtaaaggctcatgaatatggatgacactggaatt
ttataaattctaaaataaaacccaaaaccagaaaaaaaaaaaaaaa (SEQ ID NO: 49) describes
high density lipoprotein (HDL) binding protein (Hdlbp), mRNA. Gene name is
Hdlbp high density lipoprotein (HDL) binding protein. NCBI reference
sequence is NM_133808.2.
38.
Ctcaaagaggtaagctccacccttaagaagcttcacaggccccattagaggacccaccccatagactatggcctt
catgtatacgaaaggcttcgcttccttcacagagaaaggaaataatgaacagaaattatttctgagagtactggg
cacattaccaaccctgaggcaagaagagaaaatcaaagggccagaatcagtgcagtgatgtggcacccagctgta
cagagagtggagttttctggctatgtcctggaaaacactatattggaagcaaatgacaaagtgccctgtttgagt
cagttttaccagaacagtcctgtgaatgtgtaagtgcatactgccagaagtgtgtatccagtttgtgtacaaaga
actgttttaatatatatacacatacatacacagactaggggctaatgtcttttattaaatt (SEQ ID
NO: 50) describes 11 days embryo whole body cDNA, RIKEN full-length
enriched library, clone: 2700084L06 product: unclassifiable, full insert
sequence. Gene name is Cdk19 cyclin-dependent kinase 19. NCBI reference
sequence is AK019224.1.
39.
ctgcctcgcagccgtcgggatggaggtgagacggtagccgcgacgcccgccctcgcgcacccctgcaccacagcg
gcctgcagagctccccgccccccgcccccgcggcagcgggccttgccgtcgagtgacagcggcctgggggggcag
ggggggcgggggcggccagaccagcgatgccggcgggcatgacgaagcatggctcgcgctccaccagctcgctgc
cgcccgagcccatggagatcgtgcgcagcaaggcgtgctcgcgccgggtgcgcctcaacgtcgggggcctggcgc
acgaggtgctgtggcgcactctggaccgcctgcctcgcacgcggctgggcaagctccgggactgcaacacgcacg
actctctgctccaggtgtgcgacgactacagcctcgaggacaacgagtacttcttcgaccgccaccctggcgcct
tcacctctattctcaatttctaccgcaccggccggctgcacatgatggaggagatgtgcgcgctgagcttcagcc
aggagctggactactggggcatcgatgagatctacctggagtcctgctgccaggcccgctaccaccaaaagaagg
agcagatgaacgaggagctgaagcgggaggctgagacgctgcgggagcgggagggcgaggagttcgacaacacgt
gctgtgctgagaagaggaagaaactttgggacctgctggagaagcccaactcatcggtggccgccaagatcctgg
ccatcatctccatcatgttcattgtcctctccaccattgccctgtcactcaacacactgcctgagctacagagcc
tggacgaattcggccagagcacggacaacccgcagctggcacacgtggaggctgtgtgcatcgcgtggttcacca
tggagtactt
gctgaggttcctgtcctcgcccaagaaatggaagttctttaagggccccctcaacgccattgacttactggccat
cctgccctactacgtcaccatcttcctcacagaatccaacaagagcgtgctgcagttccagaatgtgcgccgtgt
ggtccagatcttccgcatcatgcgcatcctgcgcatcctgaagttggcccgccactccaccggtctgcagtcctt
gggcttcacgctgcgcaggagctacaacgagctgggcttgctcatcctcttcctcgccatgggcatcatgatctt
ctccagcctggtcttctttgccgagaaggatgaggatgacaccaagttcaaaagcatccccgcctctttctggtg
ggctaccatcaccatgacgaccgttggttacggagacatctaccctaagactctcctggggaaaatcgtgggggg
cctctgttgcattgccggtgtcctggtgattgccctccccattccaattatcgtcaataacttctccgagttcta
caaggagcagaagcgccaggagaaagccatcaagcggagagaggctctggagagagccaagaggaacggcagcat
cgtgtccatgaacatgaaggatgccttcgcccggagcatcgagatgatggacatcgtggtggagaaaaatggaga
gggcgtggctaagaaggacaaggtgcaggataaccacctgtctcccaacaagtggaaatggaccaagagggcgct
ctccgagaccagctcgagcaagtcctttgaaaccaaggagcagggatctcctgagaaggccaggtcctcgtctag
tccgcagcacttgaacgtccagcagctgcaagacatgtacagcaagatggccaagacccagtctcaacccatcct
caacaccaag
gagatggcgccgcagagccagccgcaggaagaactggagatgggcagcatgcccagccccgtggcccctctgccc
acgcgcacagagggcgtcatcgacatgcgcagcatgtctagcatcgacagcttcatcagctgcgccacggacttc
cctgaagccaccagattctcccacagtcctctggcatccctctccggcaagtctgggggcagcacagccccggag
gtgggctggcggggggctctgggtgccagcggcgggagactcatggagaccaaccccatccccgaggccagccgc
tctggtttcttcgtggagagcccccggagttccatgaagacccacaaccccatgaagctgcgagcgctcaaggtt
aacttcctggagggcgatcccaccccgctgctaccggctctgggcttgtatcacgatcctcttaggaacagagga
ggcgcagcggctgcagtcgccggactggagtgtgcctccctcttagacaagcccgtgctgagcccggagtcctcc
atctacaccacagcaagtgccaggacgccccctcgctccccagagaaacacacagcaatagcgttcaacttcgag
gcgggggtccaccagtacatagacaccgacactgatgacgagggtcagctgctctacagcgtggactccagtcct
cccaagagtctccacgggagtaccagccccaagttcagccttggagctagaacagaaaagaaccacttcgagagc
tccccgttgcccacctcccctaagttcttaaggccgaactgtgtctacgcctcagaagggttacctgggaaaggt
cccggggcccaagagaaatgcaagctggagaaccacacctcccccgatgtgcacatgctgcctgggggaggagca
cacgggagca
ctcgggatcagagtatctgagctccctaccacagagggtatttgcaggagacatcgaggaggagggatgcttggc
ctgcctgccgctgagttctgcgtgaactcggagacccaaggaccccacacacacacaattttccccacccccgca
gagccccagcgacaggagagaccctgagagccacgacaagtctgtggacaccaactaggatggggaccacaccta
acagcttcttccttagatggcaggagcagagctgacagccacatactcctagtctcctttagaggctctgtctgc
ctctgagtgcgtcatcgccatctctcatggcgtcagctgaggcaggtgttggagcaaggggttgctagtgagaga
gtgggctggccaagcagtagtgggtgtcccccctcttgttagaggggtacagaggacacttgatcctgagggagt
ctccacttcatcagccacactcctagggcataacgcttaaacaagcagcggctcaaatgattcagtattctgtgg
tatctggggctttagtctgagaagctgtcaggaggagctgacaatttaaactcaatcctttctgctcacctggca
atccagaaagggttccaacagttggcacgcagccaaggggatataagccagggctgtcacaggaatgcgggtccc
aaacagggctagcccggactgaaaagataagcgtcaaatatgcaaacgagatagattattcaaaaagactgtgtg
tgactgaaaaacaacattaaaaaaataataaatttcattttctacatgaaaaaaaaaagaaaaaagaaaagaaaa
atccttcactgaactgccctgtgaagaattgtgactactttctgctgtggtgtggtagggaacacacctactatg
agaggaagtt
ctctctctctttttttttctttctttttttgtttcctgtagtattcaagctaaaaaataataataataataagtt
aataaaaaaaaaaaaaaa (SEQ ID NO: 51) describes potassium voltage gated
channel, Shab-related subfamily, member 1 (Kcnb1), mRNA. Gene name is Kcnb1
potassium voltage gated channel, Shab-related subfamily, member 1. NCBI
reference sequence is NM_008420.3.
40.
ggtccccgcgccgcggccgcgccaatccccgcgccgccgccgccgtcgccgctgtccctgccaccgccgatctag
cagcagctctaaccccggctccggctacccagcgcacgtccgcagcccaggtcattggcgagcctaagctcagcc
ccctcccggagcccgagcccggagcccggcgcccggagccagccccagccccgcggccgccgcggctgcggggag
agggcgggggcgatgctgccggagccgccgccgctgccgccgcctcgatgagagccgcgccgcaccgcgcatagc
cgcgcaggctgacaggcagaaagactgacttccctctcccgggcatcctccctgggctgccgggaggcggcggcg
gaggaggaggaggagggaaaaggggagaaggcggagagcaggaacgagaggaggtggacctggatccgtttctcc
gggccaggactcgagcggccccagctaccgctacccgccggcgccgtcccctatccatcaaccctcctgcaccca
tccgcgaccctgggctctctgcgcgtcgggccggggctggagcggcgcggccgcagactgtctggcttccccact
atgcccatgctttgctaagtgctggtggccatcgaagtttttgcatcctggggacgaatcctgagctgaccagag
acggacggcgcaaggtccgggctctggttccctgtgagaagctgccacggcccaccgagatgtccaggcaccaca
gccgcttcgaaagagattaccgcataggctgggaccgccgtgagtggagcgtcaatgggacccacggggccacca
gcgtctgcagcgtcacctccggtgctggtggcagcacagccagcagtctcagcgcaagaccgggcctcctaccac
tgcctgtggt
accctcccggctgcccacacccgccacggctcctgctccctgcacgaccggcagcagtgaagctatcaccagtct
tgtggtcagctcggcctccgcagccaccaccaaggctcccggcatctccaaagctgacaatcagtctcaaggact
cacgaccagcatccgatgggggcagacgcctgtcaatcagtccacgccctgggacactgatgagccgccctccaa
acagatgagggagagcgacaatccaggcacagggccatgggtgaccacggtggccgctggaaaccagccttccct
gatcacacactcctatggagtgacccagcctcccaccttcagtcctgctgtgaacgtccaggcccccgtcatcgg
ggtgactccctcactgcctccccatgtgggaccccagctcccgctgataccaggccactactcactcccacagcc
gccctcccaaccgctgagcagtgtggtggtgaacatgcctgctcaggccctgtatgccagccctcagcccctggc
agtatccactttgcctggagtagggcaggtatcccgcccgggacccacccctgtgggcaacggccacatggcagg
gcccctgctgcctccacctccaccagcccagccgtccgcagctctccccagtagtgttcctgccaccaatgggcc
gcccacaacagactcggcccatgggctgcaaatgcttcggaccattggagtggggaagtatgagttcaccgaccc
agggcaccctaaagaaatgttgaaggaattgaaccagcaacgcagagcgaaagcatttacagacctgaaaattgt
tgttgaaggcagagagtttgaagtccaccaaaatgttctagcttcctgcagcttgtatttcaaggacctgattca
aaggtcggtg
caggacagcagccagagcagccgggagaagttggagctggtgctctccaacctgcaggctgatgtcctggagcta
ctgctagagtttgtctacacggggtcgctggtcatcgactcagccaatgccaagactctactcgaggcggccagc
aagttccagtttcacaccttctgcaaagtctgtgtgtcctttctagagaagcagctgactgccagcaactgtctg
ggagtgctggccatggctgaggccatgcagtgcagtgagctctaccacatggccaaggccttcgcgctgcagatc
ttccccgaggtggccgcccaggaggagatcctcagcatctctaaagatgacttcattgcctacgtctccaatgac
agcctcaataccaaggccgaggagctggtctacgagactgtcatcaagtggatcaagaaggacccagcaacacgt
gcacagtatgcagcagagctcctggctgccgtccgcctcccgttcatccatcccagctatctgctcaatgtggtg
gacaacgaagagctgatcaagtcatcagaagcatgtcgggacctggtcaatgaggctaaacgctaccacatgcta
ccccacgcccggcaggagatgcagacaccccgaacccggccccgcctctctgcaggtgtggccgaggtcatcgtt
ttggttgggggccgtcagatggttgggatgacccagcgctcactggtggctgtcacctgctggaacccacagaac
aacaagtggtaccccttggcctcgctgcctttctatgaccgcgagttcttcagtgtagtgagtgctggggacaac
atctacctctcaggtggtatggaatcaggggtgacgctggctgatgtctggtgctacatgtccctgttggataac
tggaacctgg
tctccagaatgactgtccctcgttgtcgccacaatagtctcgtctatgatgggaagatctacactcttgggggac
ttggagtggccggcaacgtggaccatgtggagaggtacgacactatcactaaccaatgggaggcagtagcccctc
tgcccaaggcggtacattcggcagcggccactgtgtgtggtggcaaaatctacgtgtttggtggcgtgaatgagg
caggccgagcggcaggtgtcctccagtcctatgtgccacagaccaacacgtggagcttcattgagtccccaatga
tagacaacaaatatgcacctgctgtcaccctcaatggctttgtgttcatcctgggaggagcttatgccagagcca
ccaccatctacgacccagaaaaaggcaacatcaaggcggggccaaacatgaatcactctcggcagttctgcagtg
ccgtggtgctcgatggtaaaatttatgctactggaggcatcgtgagcagtgagggaccagccctgggcaacatgg
aagcctacgagcccacaaccaacacatggaccctcttaccccacatgccctgtcctgtgttcagacacggctgtg
ttgtgataaaaaagtatattcaaagcggctgacgtcagcagaaagcccaagacaagtctatgggacaagtccagt
gaggcgaatgccacacaacatggttatctttcaacgccaacgagaggcccacaaaacacaatcgagaaactcact
gcgctaacatttcgaatattctctacattgaatgtagaaaatcatcctcgccttttgggtgaagaggggcacagt
gctccgggcagcaggaacctggaatgcacggagcccgggctcccaggccagcgaggcacttgctctgaacagagg
gcgctcgctc
tgcccggtgcaatagtttcacatatttttcaactgggagagaagctgtttttttccttcttgcagagcaagcttg
atccctaaaccaccatagatcagttatcctgtgacaatttggggcatcacgctcttttggaataaggtcaaagtg
tccttatcactttgattcctactttttttttttttttttggagttttttgggttttgtttttgtttttcttaaac
ctatctatactttcaagtggccgagagaaaaaggaggaatggaactgtgcagcaccaaggcctcggagagcgcct
ggtgccactagacaggatggagggccagctgcacgtgcagagccagggctggcagccgcaggggtcgctgggaag
agcagccagggtcggcagccgccagggtcgctgggaagagccgcctgtccttcatggctttttctaccatgtttt
tgctgcaaaagaacatggtgggtatgctagctttctcttagccagtatgaattatttagatttccaaggcatttt
cttgatacacaaaaggctatttttaagtaccgagagaaggaggccacaggagggacaacgctgtgggacttccca
aagctctttgtaggtagcgccagaggtagggcccttgctctcatttttctatgtgcagaatagaggatctctcct
gaggtgggccatgccccccactttatttttggaaaaagttaactcccagacagtcctggaagaccatgccatgtg
gaggcattgtcagagggaagggtctattctagaagaacatggttctggcttctgacaacagtccagctccctcca
tgaggtaaagctgacgacccaggccccgggaaagggactgagggagggagggagggagaagaaatgtctacaaag
cacaggagac
tatttttgatatttatagctatatattcaaggcacctgccatgagagctctcaggatggggacagccttcttaga
cgagccatgacagtcacggcctgagaggcacaagcagaaacactcttcttgttgcctttctgcatagtaacctga
gaaaagggaaggcaggagaggtcccagcgaggctcaaaagagaaaggccttccctttagtccttagaccctcaaa
cctcacccggtcatcagtttccattccagggcaggaagaaggccgccgccaccgtgctgtttagttgaagttagt
accaaatacacaccattttgctacctggaaagtcagcttgccatcacatcatgacaagaaccattgacagagaga
aagagacactacatgctttcatcattcagtattggatgacccaaaattccagtcctaacgacaggcatcagcctc
tagcattacaagtgtggctcccacttgacaagataccgagcttcgttatgcagtttttaatattatttattattt
taaaaaagtaataagcacaaaactacatacattgtatgtcatttaaagtatttatgtcaaacagggtgcaagtgt
gaacccaaggactggagcacaaatttctaactgcctggggcagggcgaatgtgagcattggtgtgtgtctgcctc
caagggaggtgttagtggtcaacaagactccgcggtgacactgaaattccgtttctctcctcatctatcacactg
gagcaaaactggctatttctgtgaatgatataaaacagggttctctgtaatggtattgtatatagtatatgttta
ttgttaagttgttatattataataaatatatttatagatctagactcggaagccaatgcaatgtctttgtgtgcc
attgtctttg
cctcagtgacactacagtcctgttcttcctcttggtcatcaagagtgtgtatccttctacagatgtaggtttgga
tgattagagaggacccttttgcaaagggacagagacagatgaacggtaacctccagtaaggaacagctgaaagtc
aaaagccgacttagagacttagagataccatggagttgatgcagcctgttccctgagctcctatgttcttcttct
cacagcatgtaaaagccccacagcttgcttttgcccccttcacttctgcccaaaggcccccttttcttattttct
caattcatcagcctggagctacaagacacttcctccaggaagaaaatcagtgatttccatgctgagacccaggca
gggtagccttcctgcttcctcagttgtgtctaaagggttctctggtctatcacacagacccgctttgccatgccc
aggacacctaggcaagactgcttagcatgaaatacggaaatgcatctgtcaggaaggaagtttaggaagaaaggg
tagattttccagcatagtcagccacattaagcctccatgtcctccatccctaaatcgtaccaagacctagtgtct
ccttgaaagcaaaaccttaataagcaaaaaccagcacaggaggtgagaggagaggttctactggaaactctgttg
aaacccttgggacaagaggaccattctcctgtcaaatcagtactttcctaggaacaactgtcctacaagggtgga
cacagactgtctagaagaagccaatcgtgaaggggaaggtgataatctgtgataaaaacagtctgccagctccat
tattaactatttcatttgtttaagtgaaggaacatgttcttgtggtccatgaagaatctctgtgttttataccta
actctgtgtg
gtaaagagatcccattgtaccatagacagacagctgagcacacgggagcaggagacccgccatggggactgtcaa
gacacggctgtctgatctaacaaagatgatgagaacagtgcacactgtgtgcatagattttttattttaaagaac
ttcaaaataagagtgcaatcaggggaagtacctcctgtgtgataaacagtatcttcattaaagaaaataggtaat
gtaattggaaaatattagttaagaaataggtgttaggcctaaaagaaaaaaaaaaggtattagcaaacggaatcc
aaaagtaaaaaaccaagttcaacactaaaatctggcatgaatcggtaacacctcatagcttttgaataaccatag
gggaatctgtgctcaggaggagcaggagactccaacgtagtcctgagggctctgcagagggacagacagcaaggc
cgcaggcaccgtgtatcatgagctggaggagcactgagaaatcacttctagacagccataaagaaatgacatgaa
ctctgggaaaataatgccaactattctattacaatggcgcgtgaaaagtgaccggtacttgtttgtcgacactaa
tgatgcgctgaccaccatgctaagctctcaaagccctgaatcctcaagattcaggtacaaaagtcatacacacaa
aaa (SEQ ID NO: 52) describes kelch-like 29 (Drosophila) (Klhl29), mRNA.
Gene name is Klhl29 kelch-like 29 (Drosophila). NCBI reference sequence is
NM_001164493.1.
41.
cgggaggcggctagtgagcgggcgggcgtgggctgctggtggccggacgggtcacgctgcgggtttcgtc
gccgccgccgccgccgccttgcaccattgcaccatgtccggccagctggagcgttgcgagcgcgagtggc
acgagctggagggagaatttcaggagctgcaggagacacacaggatctataagcagaagctggaggagct
gacctccctgcagacgctgtgcagcacctccatcagcaagcagaagaggcacctgaaggacctgaagcac
acactccagaggtacaagcgccattccagccatgaggaggcggcgctcatccaacagatgaccgccaaca
ttaaggagcgacagaatgtcttctttgacatggaggcctacctaccgaagaagaatgggctctacttgaa
tctggttcttggcaacgtgagcgtgaccctcctcagcaatcaggccaagtttgcttacaaggatgagtat
gagaagtttaagctctacctgaccataattcttcttctgggtgctgtggcgtgtcgatttgtccttcatt
acagggtgaccgatgaagtcttcaacttcctgctcgtgtggtattactgcaccttgaccattcgagagag
catcctcatcagcaatggttccagaatcaagggctggtgggtgtctcaccattatgtctccacgttcctg
tctggagtgatgctgacttggcctaatggactaatttaccagaagtttcgaaatcagtttttagcgttct
ccatttttcagagctgcgttcagtttctacagtactattaccagaggggctgtctctacagactgcgggc
cctgggggagaggaaccacctggacctcacagtggaagggttccaatcctggatgtggcggggcctcacc
ttcctcctgcctttcctcttctgtggccatttctggcagctctacaatgccgtcaccctgtttgagctct
ccacccatgaggaatgcaaagaatggcaggtattcgtgctggctctcaccttcctcatcctcttcctggg
caattttctgaccacgctcaaagttgtccatgccaaactccacaagaacagaaacaagacaaagcagcca
tgaacctccggacccttgtgccacctgggggccttgggacctaagactgctgggcgttccagtcagcacc
ctggctgccggtcagtggtagccatgggcagcaacatctcaggggccagcagcatcaccgggagcagggc
caaggccccggtccccggcggacatgaggaatgtgaaggccacctgcgcgcgcagtattaacctgtccca
gctccctatttataacatatttaatatcgggtcttccctgcttccctggaatccgaggcctgccttccct
ggagctctgtgtaatggccgtgggcaggcctgtgtctgtctttcagggaggggccagaagcctcagggaa
cccctctcttcctactcctatcatttgaatccctcactggggtttagatgtgcctcactcgagttggctt
tagccgacctgctactttactgtgtcccaggcaccgggaggaatgaaactttgagcatcctgtggagcct
cccctgtcctgggccaccacctcttctgtaagcctcccacagccccaccttctgcggtggacaaatattt
aagttctcagagaacctaaaaaaaaaaaaaaaaaaa (SEQ ID NO: 53) describes trans-
membrane protein 120B, mRNA (cDNA clone MGC: 31087 IMAGE: 4038363),
complete cds. Gene name is Tmem120b transmembrane protein 120B. NCBI
reference sequence is BC022593.1.
42.
cccacgcgtccgcccacgcgtccggtgcatcttaaccgaccattgtcttgaattctagagtggatcatga
cccatgaagagcatcatgcagccaaaaccctggggatcggcaaggccatcgccgtgttgacctctggtgg
agatgcccaaggtatgaatgctgccgtcagggctgtggtccgagttggtatcttcacgggcgcccgcgtc
ttctttgtccatgagggttaccaaggcctggtggacggtggcgagcacatcagggaggccacgtgggaga
gcgtgtctatgatgcttcagctgggtggcacagtgattggaagtgcccgatgcaaggacttccgggagcg
agaaggacgactccgggccgcccacaacctggtgaagcgggggatcaccaatctgtgtgtcatcggaggc
gatggcagcctcactggggctgacactttccgttcagagtggagcgacttgctgaatgatttccagaaag
acgggaagatcacagccgaggaggctacaaagtccagctacctgaacatcgtgggcctggttggctcaat
cgacaatgacttctgtggcactgatatgaccattggtactgactctgccctgcacaggattgtggagatc
gtagacgccatcaccaccacggctcagagccaccagaggacgtttgtgttagaagtgatgggccgccact
gtggatacctggcccttgtcacctctctgtcctgtggggccgactgggtttttattcctgagtgtccgcc
agacgatgactgggaagaacacctttgtcgccggctcagtgagacaaggacccgtggctctcgtctcaac
atcatcattgttgctgaaggtgccatcgacaagaacgggaagccaatcacctcagaagacatcaagaacc
tggtggtgaagcgccttggatatgacacccgggtcactgttctgggacatgtacagcggggtgggacacc
atcagcctttgaccggatcctgggcagcaggatgggtgtggaagcagtgatggcacttttggaggggacc
ccagacaccccagcctgtgtggtgagcctctctggtaaccaggctgtgcgtttgcccctcatggagtgcg
tgcaggtgaccaaagacgtgaccaaggctatggatgagaagagatttgatgaagccattaagctgagagg
ccggagcttcatgaacaactgggaggtatacaagcttctagctcatgtcagacccccagtctctaagggt
gggttgcacacagtggccgtgatgaatgtgggggccccagctgctggcatgaatgccgctgttcgctcta
ccgtgaggattggccttatccagggcaaccgggtgctggtcgtacatgatggctttgagggtctggccaa
aggtcagattgaggaggctggctggagctatgttggaggctggactggtcaaggtggttccaaacttggt
actaaaaggactctccccaagaagaacctggaacagatcagtgccaacataaccaagtttaacatccagg
gccttgtcatcattgggggctttgaggcttacacagggggcttggagctgatggaaggcaggaagcagtt
tgatgagctctgcatcccgtttgtggtcattccggccacggtttccaataacgtccctgggtcagacttc
agcatcggggctgacacagcactgaacaccatctgcacgacttgtgaccgaatcaagcagtctgcagcag
gcaccaaacgtcgggtgtttatcatcgagactatgggtggctactgtggctacctggccaccatggcagg
gctggccgctggggctgatgctgcctacatttttgaggaacccttcaccattcgcgatctccaggtgaat
gttgaacatctggtgcagaagatgaaaacaactgtgaagagaggcctggtgctgaggaatgagaaatgca
atgagaactacactactgacttcattttcaacctgtactctgaggaggggaagggcatctttgacagcag
gaagaacgtgcttggccacatgcagcagggtggaagcccaactccctttgacaggaattttgccactaag
atgggtgctaaggctatgaactggatgtctgggaaaatcaaggagagttaccgtaatggacggatctttg
ccaacacccctgactcaggctgcgttctggggatgcgtaagagggccctggtctttcagccagtaactga
gctgaaggaccagacagactttgaacaccgaatccccaaagaacagtggtggctgaagctgaggccaatc
ctcaaaatcctagccaagtacgagattgatctggacacctccgaccacgcccacctggagcacatttcaa
ggaaacggtctggagaagccgccgtctagacctctctggagtgagggggaaagatcatgtgaccatggtc
agctcacaccctggtagatcatggcttctcagtgttctagcccagcccctccccttctaggtttccctgt
actccatgcctgcaaccaggatcagctgtggccaggagctggaggaggggcggtgagtgactctcctagg
tggctgtccttcctcccttccactccagcttcatcggtcacacaggctgggtctctctagtgctacggct
agatttcagctactcaagagttttcctaaaaataagctttatttatttctttgtgataacaaagagtctt
aggtccctttactcgcaccacagtgatgaagtgtaactacactaataaatgccagctggccactgtgttt
taaaaaaaaaaaaaaaaaaaaaaaaaaaaaaaaaaaaaaaaaaaaaaaaaaaaaaaaaaaaaaaaaaaaa
aaaaaaaaaaaaaaaaaaaaaaaaaaaaaaaaaaaaaaaaaaaaaaaaaaaaaaaaaaaaaaaaaaaaaa
aaa (SEQ ID NO: 54) describes phosphofructokinase, muscle (Pfkm),
mRNA. Gene name is Pfkm phosphofructokinase, muscle. NCBI reference
sequence is NM_021514.2.
43.
ggtttaccggcggagcggccgttataaattataggaagattctgccaaagactgaactgtcttgactttc
agatactgcatagttcaaggtcagcctcaattcctgggtctcatgaatgaaacaatggctacagattctc
ctcggagacccagtcgttgtactggcggagtcgtggtccgccctcaggccgtcacggagcagtcctacat
ggagagcgtcgtgacttttctgcaggatgttgtgccacaggcttacagtgggtctcccctaacagaagaa
aaggagaagatagtctgggtcagatttgagaatgcagatctgaacgacacatcacagaatctagaatttc
atgaactgcatagcactggaaatgagcctcctctgctggttatgatcggctacagtgacggaatgcaggt
ctggggcatccctatcagcggggaagcccaggagctcttctctgtacgacatggtccagtccgagcagct
agaatcttgcctgctccacagttgggtgcccaaaagtgtgataactttgctgagaaaagacctcttcttg
gtgtttgtaagagcattggatcttcaggcacgacaccaccgtactgctgtgtggatctgtattcactccg
gactggggaaatggtcaagtccattcagtttaaaacacccatttatgatcttcactgtaacaagcggatc
ctcgtcgtagtcttgcaggagaaaattgctgcctttgatagctgtactttcacaaaaaaattttttgtca
caagttgttatccttgtcctgggccaaacatgaatcccattgctcttgggagccgctggcttgcttatgc
agaaaataagctgattcgctgtcaccagtcccgtggaggagcctgtggagacaacattcagtcttacact
gccactgtccttagtgctgctaaaacactgaaaagtggcctgacgatggttgggaaagtggtgactcaac
tgacaggcacactgccctcaggggtgacagaagatgacgttgccctacactgcaactcaagacggagccc
cttggttccaggcatcatcacagtcatagacactgagacggttggagagggccaggtgcttgtaagtgag
gactcggacagtgatggcattgtggcccacttccctgcccatgagaagccagtatgctgtatggctttta
atactagtggaatgcttttagtcacaacagatactcttggccatgactttcatgtcttccaaatcctgac
tcatccttggtcatcatcacagtgtgctgtccatcatctgtacacacttcacaggggagagaccgaggcc
aaagttcaggacatctgtttcagccatgattgtcgttgggtggtggtcagtaccctccggggtacgtccc
acgttttccccatcaatccttatggtggccagccttgtgtccgcacacacatgtcaccacgagtagtcaa
tcgcatgagccgtttccagaaaagtgctgggctggaagagattgaacaagagctgacatctaagcaaggc
ggccgctgtagtcctgttccaggcctgtccagcagcccttctggctccccgctgcatgggaagctgacca
gccaagactcttacaataactttacaaacaacaaccctggcaatccccggctttctcccctccccagctt
gatggtagtgacgcctcttgcgcaaatcaaacagccaatgacactgggaaccatcacgaagcgaacaggg
ccttatctctttggagcggggtgtttttccataaaggctccatgcaaagttaaatcacctccacaaattt
ctccaagcaaatcaatgggcggtgagttttgcgtggctgcagtgtttgggacttccaggtcgtggtttgc
aaacaacgctggtctgaaacgggaaaaagatcagtccaaacaagttgtagttgaatctctttacatcatc
agctgctacgggaccttagtggagcacatgattgagccccggccgatcagtactgctcccaagataagtg
acgacacaccactggagataatgacgtcaccccgtgccagctggacacttgttagaacgcctcagtggaa
tgaactgcagccgccattcaatgcgaatcaccctctgctcctcgctgcagaagcagtgcagtattaccag
ctcctgctggccggctctcttccccctggaagtcctggccccattactcgacatgggtcctatgacagct
tagcctctgaccatagtggacaggaagatgaagaatggctttcccaggttgaaattgtgacacacaccgg
accccacaggcgcctgtggatgggcccacaattccatttcaaaaccatccaaacctcaggccagaccacg
gtcatatcaactagttcttctgtgttacaatcgcacggacctagcgacacaccacagccgctcttggact
ttgatacggatgatctagatcttaacagcctcaggatccagcctgtccgctctgacccagtcagcatgcc
agggtcatcccgtgctgtctctgatcggaggggagtttccacagtgaccgatgctgcctcaggtaccttt
gaccggagcgtgaccctgctggaggtgtgtgggagctggcctgagggcttcgggctgcggcatatgtcct
ccatggagcactcagaggagggcctccgggaacggcttgcagatgccatggccgagtcaccaagccgaga
cgttgtgggatctggaacagaacttcagcgagagggaagcatcgagactctgagtaacagctccggctcc
accagcggcagcatcccaagaaacttcgatggctaccgatctccgctccctaccaacgagagccagccgc
tcagcctcttccccactggcttcccctaggcaccagccacctgcttctgactggctggcccgctcacccc
tagagggactgactgggaggggggagtacccccatctccctacaccaccaccaccacccctgtcccgcac
gcctctgttcccacccttttcagtctcctcaaccccaccttccctaagcttagtggcaacagctgttcat
cccaaactggacgagaagagaccttcagaacacctcggtacaccctgacctcccccactcctgtcagtgg
gggctgtggcccaaagagactgcagaaactcagtcccttctctctttgcacacaacgtcccgcactggat
ccgcttttgtattttctaactactcacaggggactggaacaggggccagatcttagggcctctggtaggg
gagagttgaaagatgcagccaggcattttggtggacatcagccctagcccagttcaggctgggatgcaca
ttgcccccaggtacccctgctcagttctaaaacaggagtgaactgagacctagagcgatagagaggaaga
aggaaagaggcctccccttgcccttgcttccctgttggctcatggagagatttgtctgtcttatctttaa
gcccttgtcccctggtcctgtactgttccatgaagggaagtgcagttaccgaggccgtgtcaaaagtgca
cgtcttgtccaataaatctcgctgcaattg (SEQ ID NO: 55) describes breast
carcinoma amplified sequence 3 (Bcas3), mRNA. Gene name is Bcas3
breast carcinoma amplified sequence 3. NCBI reference sequence is
NM_138681.2.
44.
cgcagacggagcgaggcggcggcggcggcggggcggcgcagggcgcggggcggcatggccaccaccgcgc
agtatctgccgcggggccccggcggcggagctgggggcacagggccgctcatgcatcccgatgccgccgc
ggcggcggcagcggcggccgagcggctgcacgcgggggccgcgtaccgcgaagtgcagaagctgatgcac
cacgagtggctgggcgcgggcgcgggccaccccgtgggcctagcgcaccctcaatggctacccacgggag
gaggcggcggcggcgactgggcgggcggcccgcacctggaacacggcaaggcaggcggtggcggtaccgg
ccgagctgacgacggcggcggtggcggcggtttccacgcccgcctggtgcaccaaggggcggcccacgcg
ggcgcggcatgggcacaaggcggcacagcgcaccacttgggccccgccatgtcgccgtcgcccggggccg
gcgggggtcaccagccccagccgctcgggctgtacgctcaggcggcctaccccggtggcggcggcggcgg
cctggccgggatgctggcggcgggaggcggcggcgcgggacccggcctgcaccacgcactgcacgaggac
ggccacgaggcacagctggagccgtcgccaccaccgcacctgggcgcacacggacacgcacacggacatg
cacacgcgggcggcctgcacgcggcggcggcgcacctgcacccgggcgcgggcggtggtggctcgtcggt
gggcgagcactcggacgaggatgctcccagctccgacgacctggagcagttcgccaagcagttcaagcaa
cgacgcatcaagctgggcttcacccaggccgacgtgggactggcgctgggcaccctctacggtaacgtgt
tctcgcagaccaccatctgccgtttcgaggccctgcagctgagcttcaagaacatgtgcaagctcaagcc
gctgctcaacaagtggctggaggagaccgactcgtccagcggcagccccaccaacctggacaagatcgcg
gcgcagggccgcaagcgcaagaagcgcacgtccatcgaggtgggtgtcaaaggcgcgctcgagagccact
ttctcaagtgtcccaagccgtctgcgcacgagatcaccggcctggccgacagcctgcaactggagaagga
ggtggtgcgtgtctggttctgcaaccggcggcagaaggagaagcgcatgacccccgcggccggcgcgggc
cacccgcccatggacgacgtttatgcgcctggggagctggggcctggcgggggcagcgcgtcgccacctt
ctgcgcccccgccacccccgccggccgcgctgcaccaccaccaccaccacacactgcccggctctgtgca
gtgaccctgcggactgggttccccgccggcgcagcggtgcctccggcgcgcagttagcgcgcgcggcctg
gactctttttgttgtttattcggttttgctttggattttacaaaaag (SEQ ID NO: 56)
describes POU domain, class 3, transcription factor 1 (Pou3f1), mRNA.
Gene name is POU domain, class 3, transcription factor 1. NCBI refer-
ence sequence is NM_011141.1.
45.
ttttgccaagctccgggacttcatcacgttgcgcctgcctcctggcttcccagtcaagattgaaatcccg
atttttcacatcctcaacgcccgcatcaccttcgggaacctcaatggctgcgatgagccagtgccctcgg
tgcgaggtagccccggcagcgagacgccctcccctggcagtgactcttccagcgtcagcagctccagctc
taccacctcctgtcgtgcctgtgagatctcccctgcactgttcgaggccccacgtggctacagtgtgctg
ggcggccagcgagaggctgtgcctcgtgatgaagatgatgacctgctgcggtttgccatccagcagagcc
tgcttgaggcgggcagtgagtatgaccaggtcaccatctgggaggcgctaaccaacagcaagccaggcac
ccaccccatgtcctatgaaggccgccgacaggacaggtcagtgcccacgaagccaaagagagcctagagg
tgcctgttccctctcttaccccagacccacatgcacagagatcctcactaagccatcatactgcacagtg
acactggaaccctactaggcccagagaaccctcagtgccttcaacacctggtatcgagaacccgttatca
agatggcttctgccaaccccctttcctggcacagggagggtggccagtgctccaggcatgtgcagagatc
agggacctagctccctggaccttcagaccctcaacaacagacaggttggcgggacctgggatctgccatc
actgggtggggcccagacagcccagcgagactcatggcttccccaagagggtgggcaagcctgcccagcc
ctgggacctccacgatgtctcctggccccgcttacgagcaggtgtcccaggtgtgccgggagcggggagc
gctcgacaaatcctccccgcccccaggagcgccccacccacgccgcagcgccagcccatgcccccggctc
cagtgcccagccctcggcccagcccaggtcccggctccagcagccatgtgttccggagctacgacgagca
gctgcggctggctatggagttgtccgctcaggagcaggaggagaggcggcggcgagtgcgccaggaggag
gaagagttggagcggatcctgcggctctcgctgaccgaacagtagcgcctcctgctgggaccctcgccca
ctccacgcaggacccgggtctgcgcacctgccgagcggttgctggagtctggagccatcgcattgcgggt
gtagcagcgcatcaggcttgagatgccaggggtttgatacaggcgtggaatcactttccaggccagagcc
ctagaggggtgctggtctccctgctttgctgtatggtgacccctaatccgcttctctgggctcagatctg
tccaagggcggaggcaggtggtattgaaaaggaaacgaatccttgagaggagcactgtcccgtcccttgc
tccttatggctggtcctccttcttgctcactgacccctctccaggccagagccctagaggggtgctggtc
tccctgctttgctgtatggtgacccctaatccgcttctctgggctcagatctgtccaagggcggaggcag
gtggtattgaaaaggaaacgaatccttgagaggagcactgtcccgtcccttgctccttatggctggtcct
ccttcttgctcactgacccctctccaggacactgtccctgaagccttcgcatctcagctcttcacccctg
ggaggcagctgatcgccctgcgtgctgtgtcgcgctgctttgtcccagacacaaaccagcacgtcaaggg
cctagctcctccccaccccggcatttcagcgtcaagtgcacttagcggggtacccagcccccccagtccc
aacacctgtcctgtgtctcagggtggtcctagcttctccttaggcagccaggaattggaacccccatgcc
cgcagcacgtttttcttatgatcagggagtgtgcctaaggtgccccctagtttgggcaaggcctggttcc
ctcacggtgccttggggaatgggggcagcatattggcttgggggcaagcactagaaccgacacaaaaggg
ttgatgagggccctggcaccactgtcaccacccaccccttcctaccagctgctgctagccctctgtggtt
gtgcacccctcttgcccccattcaggggccgactaacaccctttatccaatggtgctaaccccggctctc
ccttttccaccctacccacctaaagaagcacaggccccacaaagcaggggccacacccttgtcctgggct
gccttctggctggcttctctctgccaggggctcagaaggggcacaaaagggggtcaagaaaagagcaaca
aagcaaaattgttcttcctcttcccttccctgatgccaggggcaccagactgattctgaggcacaaataa
aagcggtttcaaacc (SEQ ID NO: 57) describes ankyrin repeat domain 13b
(Ankrd13b), mRNA. Gene name is Ankrd13b ankyrin repeat domain 13b.
NCBI reference sequence is NM_172945.1.
46.
gccccaagcccttccgttccgctactgaatgcttgctgaacagatgtttggacgcactttgcccatcctt
gcaggttaactgtgagtggacatcatcgaagggggcagcctgcgcacccccctccaggagctacatcaga
tgatcctgacacccatcaaggcctacagctctcccagtaccacccctgaggtgcgctgccgggagccctc
actgatggagagcccaagccctgactgcaaagacagcgctgcagcagtcaccagcgccacagccagtgcc
tcagccggtgccagcggtggactgcaaccaccccagctgagcagctgcgacggagagctggctgtcgccc
ctctgccggagggggacctcccggggcagttcacccgagtcatggggaaagtgtgcacacagctcttggt
ctccagacccgatgaagaaaatataagctcctatctacagctcttagacaagtgtcttgttcatgaggca
tttacagagacacagaaaaaaagattgttgtcatggaaacagcaggtgcagaagctctttcggtctttcc
ctcggaaaacccttctagacatatcaggatatcgacagcaaagaaatcgtggctttgggcagtccaactc
cctcccaacagccagctctgtgggcagcggcatgggcagacggaacccacgacagtaccagatcgcatct
cggaatgtcccttccgcccgccttggtctcctgggcaccagtggatttgtcagctccaaccagcgccaca
ccgctgccaaccccaccatcatgaaacaaggaagacagaacctctggtttgccaaccctgggggcagcaa
cagcatgcccagccgcacccacagctccgtccagaagacccgctcgctgcccgtgcatacttccccacaa
aacatgctgatgttccagcaaccagaatttcagcttcctgtgactgaacctgacatcaacaataggctgg
agtccttgtgcctcagtatgacggagcatgccctgggagacggggttgaccggacctccacaatctagaa
actgaagatgacagtgaccgcgctggccgtgaaatggactgctgcgggcccagtgtcagccatcttcagg
gttgcagagactcttccaagacactttgcagcctttttcccctggtccctcgcccattttgattttgtga
gagcgtaggtcatccttgtaaacatatcagtagacctgcaaaaaaaaaaaaaaaaaaaaaaaaaaaaaa
(SEQ ID NO: 58) describes sterile alpha motif domain containing 4,
mRNA (cDNA clone IMAGE: 6771595), complete cds. Gene name is Samd4
sterile alpha motif domain containing 4. NCBI reference sequence is
BC049740.1.
47.
tccgtcgttgctgaggatcgctcttagttcttgagccgccttgtgccgacttgctcggtttgtttgggga
gataacttgttttgcctcagcccgcatcccctttccttgaccccttgcaatcggatgttctagatgactg
aatggagttttgagttggacttttgtgtccctgccgaaattgggcctgatcccagagcactgggggtggg
gtgtggaggtgttactgtaaaatgcaagttggataaaaagaagacctctcgccaagggccccaatgagtg
gcacacagtctactatcacggacaggtttcccctcaaaaaacctataaggcatggaagtattttgaaccg
agagtcaccaacagataagaaacagaaagttgaacgcagttcatcgcatgattttgaccccacagatagc
tcctccaagaagacaaagtctagttcagaggagagtagatccgagatctatggtcttgttcagcgctgtg
tgatcatccagaaagacgacaatggcttcgggctgacagtcagtggagacaatccggtcttcgtacaatc
tgtcaaagaagatggagcagccatgcgtgctggagtacaaacaggtgatcgaatcatcaaggtgaatggt
accctggtgactcattcaaatcatctggaggtggtgaaactaatcagatctggttcctatgtagctctca
ctgtccagggacgcccacctgggtcgccccagatcccacttgctgactctgaagtagagccatcagtcac
tggacatatgtctcccattatgacatcccctcattcccctggagcagccgggaatatggagagaatcacc
agtcctgtgctcgtgggggaggaaaacaatgtggtccataaccagaaagtagagattctgagaaagatgt
tacagaaggaacaggaacggctgcagttgctgcaggaagattacaaccgaacggctacccaaagattgct
caaggagatccaggaggccaagaagcacatcccgcagctgcaggagcagctgtccaaagccacgggctct
gctcaggacggagctgtaattgcaccctccaggccattaggtgatgccctaacactcagtgaggcagaag
cagatcctggagatggcctgtgcaggactgactggagcagtggagatgcttctcggcccagtagtgacag
tgctgatagtcccaagagtagcctgagagaaaggagttactcggaggaagccccagagagaagtgaagga
gttcaggacgctgagccgcagtcgctggtcggcagtccctccacccggggcgcccctcacatcattggag
cagaggatgatgattttggtactgagcatgagcagatcaatggacagtgcagctgtttccagagcattga
attgctgaagtctcgccctgctcacttggctgttttcttacaccatgtagtctcacagtttgaccctgca
acattgctctgttatctctattcagacctgtataagcagacaaactccaaggagactcggcgtgtcttcc
tggagtttcatcagttcttcttggaccggtctgcacacctgaaggttccagttcctgaggaaatctccgt
ggatctagaaaagagaagacctgagcttattcctgaggatctgcatcgcctctacattcagacaatgcag
gagagagtccaccctgaagttcagaggcacttggaggatttccgacagaaacgcagcatggggctgacct
tagctgagagtgagctgactaaacttgatgcagagagagacaaggaccggggaactctggagaaggagcg
agcatgtgcggaacagatcgttaccaaaattgaagaagtgttgatgactgctcaggctgtagaagaagaa
aggagttccacaatgcagtatgtaattctcatgtatatgaagtatttgggagtaaaagtgaaagaacctc
gaaacttggagcacaagcggggtcggatcggcttccttccaaagatcaagcaaagcatgaagaaagatag
agagggcgaagaaaaagggaagcgaagaggattccccagcattttgggacccccaagaagaccaagccgc
cacgacaacagtgcaattggcagggccatggagatacagaagtcacgccacccgaagcacttatccactc
cctcgtctgtgagtcctgagcctcaggaccctgccaagttgcgccagagtggagtagccaatgaaggaac
agacactggatacctgcccgccagttccatgtcctctgccacctcagggactgctctttcccaagaggga
gggagagagaatgatacaggaacaaaacaagtgggagaagcatcggcgcctggagactgcttggacagca
cacctcgagtccccactactgttttcgacttccctcctcctctattagatcaagtgcaggaggaggagtg
tgaagtagagagggtggcagaacatgggaccccaaaaccctttcggaagtttgacagcatcgcctttgga
gaaagtcagagtgaggatgagcagtttgaaaatgacctggagacagatccacccaactggcagcagcttg
ttagcagagaagtgttactgggactgaaaccttctgagatcaaaagacaggaagtgattaatgaattgtt
ctacactgagagagctcatgtccggacactgaaggttcttgaccaagtattctatcagcgagtgtccaga
gaagggattctatcaccttcagaactacgcaagattttttcaaacttggaagatatccttcaacttcatg
ttgggttgaatgagcaaatgaaagctgtccgaaaaaggaatgaaacttctgttattgatcatattgggga
agatctgctcatctggtttagtggaccaggagaggagaagctgaaacatgctgctgccacattctgcagt
aaccaacctttcgccctggaaatgatcaagtctcgccagaaaaaggattcacgattccatacttttgtgc
aagacgctgagagtaaccccctgtgccgtcgtcttcagctgaaggacatcattcccacccaaatgcagag
actcactaagtacccgcttcttttggataacattgccaagtacacagaatggcctccagaaagggaaaag
gtgaagaaggctgcagaccattgtcgtcagatcttaaattatgtaaatcaggctgtcagggaggcagaaa
acaagcagcgcttagaagactatcagcgtcgccttgacacctccaatctcaagttgtccgagtacccaaa
tgttgacgagctcaggaatttggatttaacaaaaagaaagatgattcatgaagggccgttggtctggaag
gtgaatagagataaaagcatcgatctttatacactgctgctggaagacatccttgtgctgctacagaagc
aggatgacaggctggtgctaaggtgccacagcaagattctggcatccacagccgacagcaaacacacctt
cagccctgtcattaagctgagcacggtgctggttcggcaggtggcgacagataacaaagctttattcgtc
atctccatgtcagataatggagcccaaatttatgaattggtagcacagacggtttctgaaaagacagtct
ggcaagacctcatctgtcgaatggctgcgtcagtgaaggagcagtctacaaagccgattcccctcccaca
gccgccaccttgcgaaggagacaatgatgaagaagaaccagccaagttaaaagtggagcaccatgatctt
tcagtcgctggtctgcaaagtccagacagagttttgggattagaatctcccttaatttcgtcaaagcctc
agtctcattcactgaatacccctggcaaatcagcggcagaacatctctttgtgaccgcaacacagtttgc
aaaggaacagcatgcaaatggggcactcaaggaaggtgatggcgggtatccagtcacaatcccaggtcca
cacttgcctgtgtcagaggagcggtgggcattggacgcactgagaaatttgggcttgttgaagcagttgc
tggtacagcagttaggtctcaccgagaagagtactcaggaagactggcagtccttctcacgatacgggcc
agcttctgaagaggtacaggcagacagtggcatccgggacttggaaaatgttaaggcttgtcatgccagg
gaaggacagatgtcttttaaaactggaactggtgatattgcaacttgtgacagtccacggacttcaactg
agtcttgtgctgcacaggattcagtgatactggcatcccaagacagccaggcaagtaacgttttagtaat
ggaccacatgatcctgaccccagagatgcctcctgcagagccagaagggggtctggatgagagtggagag
cacttttttgatgcccgtgaagcacatagtgatgataatccatcagaaggtgatggagcagttaaaaagg
aagagaaggatgttaatttacgcatctcaggaaactgtttgatccttgatggctatgatgcagttcagga
gagctccacagatgaggaggttgcctcctcgttccccctgcagcctgtgacgggcatccccgctgtggac
tccagccaccagcagcaacattcccctcagaatgtgcatcccgaggggccggtttcaccattcaccccag
aattcctggtccagcggcactggcgagctatggaggatacctgttttgagatccagagtccctcatgtac
agattcacaaagccaaatcctggagtacattcataaaatagaggctgacctggaacacttaaagaaggtg
gaggaaagttacgctcttctttgccaaaggctggctggctcagccctccccgacaagctctcagataaaa
gttagagccccgtgtcctggaggtgactgcagcttgttggattttgagtgtcggccctgtatcaaccaca
tcctggctccagtgtggacacagagagagtgcgacagagggatctgcctggaaccacctggcctcagcca
ggccccaggggccccgcggggactagttcttccttctggcagctgcacacatctgtcagtgagggaatgc
caggtctctcacgcctctcctcactatcagaaatgcattttgattcagagtaaaaaccaaatgtatagag
ctttgggtgtaggatataaaattgtacgtaggtaagaaaaagagaaaatcagatgtatttatttgacttc
attccgtatttgaaagcacactttaatttttactttgccttgttttgtttttaattgagtagtgagagtt
ttagccctttgtatttagcacacccaaggatcaattgttcctgaagccgaggtcaggctggggatggaga
ggttagtagacaggcagtgcagaagagcaaagaggactacctggccctcgctaatctgtaacccagccct
cctgtccagaaggctgcctttctgaggatcatctcagaggtgctgcatgactcctgtgtgtggatgccat
gagctgtgtgtgcccgggtgctcggagcaggaggacctcctcccagcccaaggcgagcatctcctactgg
gttccattcccaggaaacaggaatcctcagtacactaaatggcaggcacttgaaaatgggtggatttccc
ttgtcgttgtcttttctattgatagttgggatttgggagagaagaaggaggttttcgaaactgaatcact
actgagttgaaccatggctgtcactagccacgggtactgctgattataaggccagtaagaaaattagtac
ctggatgtttgactcttaaggttttgcactgatggtgccaaagggctctgagtcacacgcagagccaagg
gagggcagaggaaggcatgaaggagagttgctcgcatcgcatcccttcagagagctttctctgacctcac
tgccatagccctgtctgtctggtgccaggactggccttgggaacggggaccagcggtgagcagttgcagc
cgccaaggtgctgacgtgctgcagtgtggacagcgcaggtggaaggccagctggtcagactgtggcctga
caactcttaattctgatggtgttcagtggcttgctttgtgcatttgataaatatgtttttagagtgttct
gtaaaaaaaaaaaatcatttgtctaatctaaattctgttaagaaggagctcacatgttgcttacatcttg
caggagggggcctctgaactctgggggcagtcaggatgtaattttattctccccttaatgcccctcacac
aaaggaagctcactgtcgccaagaatagtcccccagctgcttttatgaacacctctacaacttaacaagc
tcatctgtctgggtccagattctcgtttctgtcctctaagctcttacgtcgattggctaggacccctgta
cctcatctataaattggaatcatattttgagtcatagtcaaccctaactcataatggagctttgagattt
tggagtagaatctaatatataagtgaagtttgggatttggacctccctgtatctcttaggagaagcaagt
agagaccgaatggttccgttgtcttccaggcagcctcttacactacaccatctgggtagctcatcagaca
gcagtgttccgacctcagacactacagatccctccgtggttctccagcacagttcagccggaactctgtt
ctcctgcttgtttacttccctctttctgagtcccagcctctcagagtcacagcagggcttactggacagg
tactctggcgttgctccagtacttccaaataaatgcatatcattacaccttcagatgaaacctcagaatg
aagtgcattctgaaagacaacagcaacaaaccactttgtaagttttttttttactctaccagccttactt
cttgcctgttcccctccctcgggcctcctgtgccttccactccattctggccattagaatccatttgact
ttgacattacatctcccttccgtggtggcgacagcagtgttcctcctgttctggaatgagatggtcattg
tcatagtcctcttaaaggtgggtgacaagaaggtgcagtgtacttctgctccaaagaacactaggcaagc
ggaaagccatgtgtgccatgggtgcaacctgtgaccctgctctcctgtgagcttagtggctctcctgtca
ctaactggcaggttatgtgtggagtgtcaccttttcagagagaaaaactaaccaagccaaaccagcaaac
cctccaagtactgtaatgtaaaggccggctttggcttccccacctgtatagtttcatggcagccccctgg
actgttgagttctgctgctttcacaacctaaactgtaactatttattgtatcacacgcaggttgttcatg
agtattttttcctttgtttccccaaacaacttcttttttttcatgaagtcctggaagcttatggtttatt
gtggattgttctcaggagagtaagagacaagaaaagcaacatatatgtggggggaggggttgaagtggtg
gtggagcaaagccagctggagacaggctccagcctagacccttgaagaccagagagcagcaatgcaccat
actcttgatttcatggggagaaagggggatggaaccccaggccaggaggtgtaactcctagctgcacctg
tttccatatgaccaagagtaatcatccgggaatgagagagtctataaagaacactcattgctgctgcagg
tgagacagacatggccaggtgacaccagccatgtctctgatttcagtcaactcttttcattagtttcctg
aatagcaaacacttccttgaaactcttctcaaacttggactccagtctaagtcccatttcgccaaactta
gggtccttgaacgtgattttgtgattccggaagatgagcctcatgtcttgcacaaagtcttctacttggc
agtaagcctggtcactcagctttttcttgattatgttcaaccacataggctcttgcacacccacagtcat
ctctctaaaataataataatatggcatcttggaaaaaaaggagctctcagaacagcaatagactctcaag
agaacatactcgcacttcaactgttcctggggagccatctccctctgcagtatctcagattctggaagac
tcggttgacttcctaaggactgcatcctacagaagatgcaactccatggtgttatctcagcttccactgt
tgggatatgacagtcctcatggaaggctctggaacaagtatcacagcagaagagtattcctccatcccgg
cacacctcacattcatctgagttctgaatactagggtcagctggggaacgatgcaagttctgtgttctct
gctcttttcttttgcgatgtatccgtggaggattgggtagaaattttctctggatcaggagtttgagggg
ccaattatggcagcgcaggctcagtttccagttctttgactttccatagcctcccatcatttcaaattcc
gagggtgtgaaccagtttccattctgacactgtatggacttcacagatatcccttgtttgaatttatcct
tgtgtaacactcccttcaagttaccacacgtcacgggaagcaactcagcactaaaatcagcattttcttt
tctgcgtcttcttagacccctgatcttagcagtgcgtcctgggttctttttcattttctttctcttgaaa
gctgcctggacaacagctattctgccacgttttctcagtacactcattggctttctccatttggtttgac
cagttgtggctttcttttttcttctctgcttcttgggttttctgcagaatgttcctcctggtgcccacac
attctccttggctgttccatcctgcttccagtcttctggcacatatggaacacatgacacaggaacactt
cttttcacgtgtgggttgctgtgctcctgggacaaaattgtgggtgtacataggtttgttatctaatttt
gtaatactggggaccaagctcagggtctcatttgtgcaaggcaagcagtctaccagtgccccaaacaact
tctaatgacctctagaaagaacaagcccttttttagaaagtattctgtttcattgtcttttggggagtga
agaagagaaaagtccttgttgccagaattttagaagctaaaagactttatgctgcctctactgcaggggc
tcgttttcccgtcgctcctgggcactggcgctggcctgctgcctgcagctggtaccctgcccaagctgtc
ttctctgcagccgcctgcttgagcttgctctgtgcctgcttccctccacgctccgaaaaccatgatctac
aaaaacagacttctgcaaggtctcgctcgcctgcccacccgccccctaacgttgtgtggttttggttttg
gttttggtttttgacagttcacccccatttagtgtgagctgtaaaactgtactttatttaaaggcgtgct
cttgttcccgtctggctgccttccccatgggtgctgagggtttgggtaatacactgatcacggaaaaggt
ggtcttctcagaaaccagtccctccctcccagccattgccaggtgccacccgctaagcagacatactaca
gcaggcgaccgtagcgtgcacactgttggtggtttttaatgagcctctctttccccactaggctgtctca
ctttacttgttcaccatacaatcatttactctttaacaaaaattgcttttaagagaaatctggaactatc
tttaaaaaaaaaaccttattaataatcatgtatttttactgatcacattttgaaatgcctaaaagacttt
tattgctctaattatccagatgcacctttgtaaaaagctcttttatgaattagctgataaggctgtatgt
ttctggaacaaaatattgggtcatctaaaactttctgttttctggggtctgggaaaatagaaaataagaa
ttcaaatattaaataagcttaaaagaactg (SEQ ID NO: 59) describes Rho guanine
nucleotide exchange factor (GEF) 12 (Arhgef12), mRNA. Gene name is Rho
guanine nucleotide exchange factor (GEF) 12. NCBI reference sequence
is NM_027144.1.
48.
gggtgacgtgttagccatggagacccaggagggtgaggaagaccgagggtgatggggaaaccgaggctgg
gacggcgcggttgttcccgtggtccgcatgcgtgggggaggacgcggctcggcctggtgtctccaaacaa
agcatacgtgaacggatttgggactacatggaatcccatgatatagctgactttccccgacccgttcatc
acaggatccccaacttcaagggtgctgctcaggctgcggggcacctcccacacctgcaagctttccatgt
ggccagaaccattaaagtcaacccggacgctcctcagagaaacgcccgcttcctggtgctggagagcaaa
aaaacactgctggtcccaacaccacgactgcgaacagggctgtttaacaagatcacgccaccccctggag
ccaccaaagacattctgaggaaatgtgccacctcccagggtgtgcggaatttcagcgtcccagtgggctt
ggattccagtgttcttgtggatctagttgtggtgggatctgttgctgtgtctgagaaaggctggcgaatt
gggaaaggggaaggctatgctgatcttgaatacgccatgatggtgtcaatgggagccgtccacaagggga
caccggttgtcacaattgtccacgactgccaggtcgtagacatccccgaggctcttgtggaggaccacga
cctcacagttgactacatcctcactcccaccagggtcatcactacaggctgtgcacgcccaaagccaacg
ggcatcatgtggtccaaggtcagctgtgagatgcttacgaaaatcccagtgctcaggaacctccgggaga
gagagaaacaggctgggaaggatgtcactctccgggacgagcctggcagccagcagccagctccaggccc
catcaggagaccccaggatagaccacaaacaggttctcgaggagggtcccgtagtcccctgcagggggcg
gacacccaactagctgccactgtgtgtgtgggaaacctgcccttcactactcgtgtgcgtgagctgaaga
gagtcctccaggagctgggggtggtgcccctgaggcttacctggcaggggccacagcacagggcagttct
acactatacagactcagctgctgcccagcaggccgcctctctcctacagggcctacgtctgggtgccaac
gccttgagggtgtcactggggcagcagagggatatgtgacctggtgagtaacccccagcagaggagtagc
gccctttctgctgctgggatgtgtctgccgtgtgacagtttgccgtgtgacagtttgccattggtgatga
ccggctgccgtgtgaggcacctgctctgttcttcctgggaagggggccatgcttccctaatgggcaggga
tgacggcgagcagcccatctcgacacccaggatgctgtgagctctggcccattcctggtctggttcttgt
gaaatccacgtgaggttctctgggtgcagaaggttcagggacagagcagtccagacaagaggtagacaat
ggagacctgtttcagggtgttcaaggagagcggaggcctgcagagaagaggccgtctcggggatcaaggg
catgtggccgagcagggtgtccttacgaccactgggaacccacttagcagaacaggagcggggagggcgg
gcttctgcatccagagaacacaaacttggagctctgttcctggagacgctgcattgtgagcatgcgtgat
ggctgctacccaggcagcttagtcatgccatctctcaggtgaacatggcggctgcctgtgtgctttcaga
agcacttgtgccgttgagcctaaggaaaaacaagcattaggtggagggtttgtcctgttcaaactgcaca
cctgggcagtttcttgtttttaccaatgggtcttccacagcgtctattagcattgtcctttgtcccaaaa
gggcaggcctcgctcatattaaaggaaaagagcttgtgacacg (SEQ ID NO: 60) describes
methenyltetrahydrofolate synthetase domain containing (Mthfsd), mRNA.
Gene name is Mthfsd methenyltetrahydrofolate synthetase domain con-
taining. NCBI reference sequence is NM_172761.1.
49.
gttggaggggctgagttgatgaaggcaggaatttagccatccggcttctcgccctctggcggctccaaac
ggcaggccgacttgctttcactacccgcatgctcctagatccacaccgagagtctccaggggggtgactt
gtcgggtgcgggcttccagatgggggatgaggggccgggaagcccggtgcatctgctgtgcctcgcagcc
tctagcggggttcccctgttttgtcggagtagcagcggcggcgccccctcccgtcaacagcttcctttct
ctgtcatcggctctctcaatggagtccacatgttcggccagaatctggatgtgcaactgaactctgcaag
aacggaagacacaaccgtagtgtggaagaacttccatgacagtatcactctcattgttctgtcatctgag
gagggcacctctgagctgaggctggaaagaatgctccacatggtgtttggggccatggttctcattgtgg
gacttgaggagctgaccaatatccggaatgtagaaagactgaagaaagaattgagggccagctactgtct
catcgacagcttcctggggaactcagaactcatcggagacctgacccagtgtgtggactgtgtcattcct
ccagaggggtctgccatgcaggtattggggcagctatagaaataactgtgtccccacggttctgctcagc
cttcctttgttttctttctctggacattgtcctaactccctcttatcatcctggaatcaagttttacctg
gaaccttctcaccagggtacactgactgcttgcctcctgtcctgcagcttcggcctcacaagcaccctgc
cttcctgccttgcagggaaccctgtctaggcaaatccgcctcaccccattctaagacagggctcccaggc
tgtcttgggactctctctgtaggccagtccggccgacctccaacttacaactcacaaacacccaccagcc
tttgcctctgagtgctgggattaaaggcgtgctccaccatgcctgcccaatcttcctttgctaaagttat
gcctctcctggcagcatgtcctgacctcagagcttccgtaggtcaggactgtgccttctcagcaagttta
gatgctgttgttaaaactattggcatcccaggacctcctggtgcaggcctgtcaccccagctactctgga
ggctggggagagtatgcaaagtttaaggccggcctaggcagctcagtgaacacatgactcaaaaattcag
tatgacttggcaggagcagccccataactgaaggagaaataaattttaagaagcaaacaaacaaacagat
aaaataagtgtatggagaattgagagtgctgactgatcaggaaggttaaagtattctcgttcacagagag
ggtgtgagacaggagcttggcagccttgcgtgctagggtgtctctggtctcatggctgctcttcccctcc
ctgactagccgggaaggtccctccttgccctatgacatctagtctgaccttttcaccaggaaaccctttc
agggtttgcagaggccactggcacagcctttgtcagcctgcttgtatcaggccgagtggtggcagcgaca
gagggctggtggcgcctaggcatgcctgaagctgtactactcccctggctggtggggtccttgccaccac
aagctgctcgagactacccggtgtatctgccccacgggagccccacggtcccacacaggttgttgacctt
gacgctgctgcgtggcctggagctgtgtctgctgtgcgggccgcgcccgccccttggccagctggaccca
cagttaatggaacgctggtggcagcctctactggagcccctccgggcctgcctgccactgggccctaggg
cgctacccgaagggttcccactacacagcgatattcttgggtaaggctcgatggttgggcccagggccgg
gaaggccatttgctctgccagttccccacagcctgatagtgctttcccccagtctgttgttactccactt
ggaactgcgacgctgcctctttactgtggagccctccaaggataaaggtgcgtgggaacgcagtgagtgg
gagggccgtgagccagaggccacctagtcaaggtgctccctcttctttagagccttcccctgagcagcgc
aggcgcctccttagaaacttctacacgctggtggccaccacccacttcccgccagaaccagggcctgcag
agaaacaagaggacacagtctacccagcccagatgcccagagcctgttatctcgtgctagggccaggcat
ggggtggcagctggtggctgtgcaactagggctccggctgctactattgctgctctcccctcacaccccc
acccacgggctgcgaagcctagccacccgcactctgcaagccctcaccccgctcctctgatcactcaggg
tgggcagtcaatagctggggtaatattagcgtctctctgtttattcgctcacaataatacacagcccctg
gatggggaaagggaggaagggggagggtgacagggaaggggggggagaggtacctacttcctcggcccct
ctcccctctgtgcttcgggggacaagggagggagggtgcttcccttgacccccccctagaatgtaaacag
cagcagatgaacaaaaataaaatacaaaaggccagagggagctcccaggcgcccccaaaaaaaaaaaaaa
aaaaaaaaaaaaaaaaaaaaaaaaaaaaaaaaaaaaaaaaaaaaaaaaaaaaaaaaaaaaaaaaaaaaaa
aaaaaaaaaaaaaaaaaaaaaaaaaaaaaaaaaaaaaaaaa (SEQ ID NO: 61) describes
fuzzy homolog (Drosophila), mRNA (cDNA clone IMAGE: 4507676), complete
cds. Gene name is Fuz fuzzy homolog (Drosophila). NCBI reference
sequence is BC045601.1.
50.
gcggcggcctggaaccggttggctgggacctgacaccgcctgcaagctgcaggttgcttctataactgga
tcacgagtttgctgcacaaagtaccatgtctcgatcccggcagcccccacttgtaacaggcatctctcca
aatgaaggaataccttggaccaaagtcacaattagaggggagaacctgggtactggtcccactgacctca
taggcttgacaatttgtggacataattgcctcctcacggcagaatggatgtctgcgagtaaaattgtctg
tcgagtgggacaagccaaaaatgacaaaggagacattattgtcacaactaagtcagggggtaaaggaacc
tcaactgtctcttttaagctactcaagcctgaaaaaataggcattttagatcagtctgcagtgtgggtgg
atgaaatgaattattatgatatgcggactgacaggaacaaagggattcctcccttgtctctccgtcctgc
gaatcctcttggcattgaaattgagaaatgtaaacttcctcagaagaacttagaagtgttattccatggc
atgagtgctgattttacaagtgagaatttttcagcagcctggtatctcatagagaatcactccaccacca
gttttgagcagctcaagatggcggtcaccaacctcaaaaggcaggccaacaagaagagtgagggtagtct
ggcatatgtgaagggcggcctcagtaccttctttgaagctcaagatgctctctcagctatccatcaaaaa
ctagaagcagatggaactgaaaaagtagaaggatccatgacacagaaattggagaatgtcctcaatagag
caagtaacaccgctgacacattgttccaagaagtattaggtcggaaagacaaggcagattccactaggaa
cgccctcaacgtacttcagcgatttaaatttctcttcaaccttcctctcaatattaaacggaatattcaa
aagggggattatgatgtagttattaatgattatgaaaaagccaaatcgctctttgggaaaacggaggtgc
aagttttcaagaaatattatgctgaagtagaagcaggaattgaagatctaagagagttacttctaaagaa
attgctcgagactccatcaaccttacatgaccagaagcgctacataaggtatctttccgacctccatgca
cctggtgaccctgcttggcagtgtattggagctcagcacaagtggaccctcaagctcatgcaagactgca
aagaaggccacatgaagagcctaaaaggtcacccaggcccgcacagccccatgctcgacctagacaacga
tgtgcgcccctcggtgttgggccacctcagtcagacagcgtcactgaagagggggagcagctttcagtct
ggccgagatgacacatggaggtataagactccccacagagtggcatttgttgaaaaattaaccaagcttg
tgttaagtcagctgcctaacttctggaaactctggatttcatatgttaatggaagtcttttcagtgagac
ggctgagaagtcaggccagagtgaaagatcaaagaatgtgaggcagagacaaaacgatttcaagaaaatg
attcaagaagtaatgcactccctggtgaagctgatccggggagccctgcttccactcagcctccgagaag
gggatgggaggcagtatggcggctgggaggtgcaggcagagctttcggggcagtggctcgcacacgttat
ccagaccataaggctaacatatgaatcactgaccgccctggagattcccaatgacatgttacagattatc
caggacctaattttggatctccgaatacgttgcataatggtgacattgcagcatacggccgaagagataa
agagattagctgaaaaagaagactgggttgttgataatgaaggactgacttccctaccatgtcagtttga
acagagcattgttcactccctgcagtccttgaagggtgttgtcgattgcaaacctggagaagccagcgtc
tttcaacagcctaaaacgcaggaggaggtttgccagttatgcatcaatatcatgcaggtttttatatact
gcctggaacaactgagcaccaagcctgatgcagacatagatactactcatctttctgttgatgtttcttc
tcctgatttgtttggaagcatccatgaagacttcagtttgacttcagagcagaggcttttgatagtcctg
agtaattgctgctatttagaacgtcacaccttcctaaacatagcagaacattttgaaaaacacaacttcc
agggaatagagaaaataacacaggttagcatggcatcgctgaaagaactggatcaacgactctttgaaaa
ttacattgagctaaaagcagatcccattgttggatccttagaacctgggatctacgcaggctactttgat
tggaaggactgcctgcctccggcaggtgtcagaaactatttaaaagaagcattggtgaatataattgctg
tgcatgcagaggtattcactatttccaaggaactggtgccacgggtcttggccagagtggtagaagcagt
ctctgaagagctgagccggctgatgcagtgcgtctcatccttcagcagaaatggagcactgcaggccaga
cttgaaatctgtgctctgagggacactgtagccatttacctgacctcggaaagcaggtctagttttaaac
aggccctggaggccctgcctcagcttgcaagtggtgcggacaaaaagtcactggaagagctgctcaacaa
gttcaagagcagcatgcacttgcagctcacctgcttccaagctgcgtctccagcagtcatgaaaacatag
gctctgcaagaagagcatcagacatggcggtggagactgtcctcgtgctgtgcagcagagattacacgtc
aagcactgcatttaacgtctcttttccccagcatggtggtgcagaaagatgcccatggatatttaatttc
aatctatttaaactaccatagtttaaacatccaaataaacagtttttgttattgctagctttggtcctga
aaggctgtgtagcatactcttcaaaatgctgtgagtctggggatttctccatcatggtccagagcggccc
atggtgtgtgtgtgtgcccttctcagtgcccctcttctaggcctactgttggcagtttccagcccaggtg
agacatttgagaagagatttgaagtcacagtttttagagctttgtcctaaactttaaaataaccatttca
tacaagtggttctaagctccccaccctgtttctgtaaagaaaaatatacagtctacacataaaaatgtta
atatttggataaatttgtgatccacaagtcctcgtatgaactctgaatttcatagtctgactttcatttc
tcccagtgctaaacactgaagcttgtggttctcactcagccttaagctgatggatcactgcagtggtgtg
gggagggttcacaaggacctgtatgtggaggccctgtcacacccacaaggcctggggaaggcttgtggtc
ttcaatgcagcctaaagatgaagccaggctcctcggcaaggcagcactccctccccctctgccacttccc
acgcggtgaaatgtcttgctccatagctggaaagaggctgctctggcatgggctgtgttttgtaaagatt
tgaggtgccccaagtgaccaggcactgagctgtgactcagtggtgagtggcttgcgcagtagagtcaggt
cattgtgacctgcaggcagctttcacctcactccatccttccgtcagcctctgttgcacttcactcttca
gactgatctgtggataatgtaggctcctcttctagagttgacaaaactaattccagagcacagcatatgt
accctgtggattatttaaggtaaatgagtgtatgtttatttatcacagtgactggaagtagaaattgtaa
tcctttgttgcaaaataagtatgtaccgcattgtctcctccctgtgtgctgttattatatctgacataaa
caaactaaattccaaagcagaagtacagaaccatttgttattttaaacacctaataaatacagtgcacaa
agccagtagttgtccttctctgtaagacccctatctttatttcaccatttttattagctctgtaattgct
tatattcctgattttaaaatccacaataaatagtggagttctctcttttaaaaaaaaaaaaaaaaaaaaa
aaaaaaa (SEQ ID NO: 62) describes exocyst complex component 2 (Exoc2),
mRNA. Gene name is Exoc2 exocyst complex component 2. NCBI reference
sequence is NM_025588.2.
51.
ccgggagcagatggctggcatcaggggtggcctctcgatcagagtggagctagagatagaggaagcgccctaggc
tgggtcgccttgagcaactggtgaaactctgcgtctggtgccccgagtgtgtcatagtccagaagtgagtgagtg
gcactcggggctaacttctcagtgtgtctgttggcaggcgaagagtcgtatggcgcttgttctttcttcagaact
gtcacgatgcatgtgtctgacgcttgtaggcgcctttagtgtttgctagttcttccctttcacgttttcaaatgt
ggcactgccgcgaacagaggctcaggcacatccgcctggtagtggaggacggagtgcatgagacaattgaggaac
caacacaagacagcagcccttcgaggtctttaggcagcttggaggagaacacatgagagaaagaatcccaagagg
ttttgttttctttgagaaggtatttctgtccagctgctccaatgacagagatacctgcacctttgtcctacttcc
agaatgcccagatgtctgaggacagccactccagcagcgccatccggagccagaatgacagccaagaacggcagc
agcagcatgacaggcagagacttgacaaccctgagccaatatctaatgggcggccccagagtaactcaagacagg
tggtggaacaagatgaggaggaagacgaagagctgacattgaaatatggagccaagcatgtcatcatgctctttg
tccccgtgaccctctgcatggtcgtcgtcgtggccaccatcaaatcagtcagcttctatacccggaaggacggtc
agctaatctacaccccattcacagaagacactgagactgtaggccaaagagccctgcactcgatcctgaatgcgg
ccatcatgat
cagtgtcattgtcattatgaccatcctcctggtggtcctgtataaatacaggtgctacaaggtcatccacgcctg
gcttattatttcatctctgttgttgctgttctttttttcgttcatttacttaggggaagtatttaagacctacaa
tgtcgccgtggactacgttacagtagcactcctaatctggaattttggtgtggtcgggatgattgccatccactg
gaaaggcccccttcgactgcagcaggcgtatctcattatgatcagtgccctcatggccctggtatttatcaagta
cctccccgaatggaccgcatggctcatcttggctgtgatttcagtatatgatttggtggctgttttatgtcccaa
aggcccacttcgtatgctggttgaaacagctcaggaaagaaatgagactctctttccagctcttatctattcctc
aacaatggtgtggttggtgaatatggctgaaggagacccagaagcccaaaggagggtacccaagaaccccaagta
taacacacaaagagcggagagagagacacaggacagtggttctgggaacgatgatggtggcttcagtgaggagtg
ggaggcccaaagagacagtcacctggggcctcatcgctccactcccgagtcaagagctgctgtccaggaactttc
tgggagcattctaacgagtgaagacccggaggaaagaggagtaaaacttggactgggagatttcattttctacag
tgttctggttggtaaggcctcagcaaccgccagtggagactggaacacaaccatagcctgctttgtagccatact
gatcggcctgtgccttacattactcctgctcgccattttcaagaaagcgttgccagccctccccatctccatcac
cttcgggctc
gtgttctacttcgccacggattaccttgtgcagcccttcatggaccaacttgcattccatcagttttatatctag
cctttctgcagttagaacatggatgtttcttctttgattatcaaaaacacaaaaacagagagcaagcccgaggag
gagactggtgactttcctgtgtcctcagctaacaaaggcaggactccagctggacttctgcagcttccttccgag
tctccctagccacccgcactactggactgtggaaggaagcgtctacagagaacggtttccaacatccatcgctgc
agcagacggtgtccctcagtgacttgagagacaaggacaaggaaatgtgctgggccaaggagctgccgtgctctg
ctagctttgacccgtgggcatggagatttacccgcactgtgaactctctaaaggttaaacaaagtgaggtgaacc
aaacagagctgccatcttccacaccatgttggaaataaaacacgtcctagctgaacccttactgtccaggaagtt
ccgtgtggaagtggcactgggccgggcctccctctcaggctcctttgctgcccacttgtaagtttaaataaggac
accgccctacacaaacctcaccctgtcacatcagtgactctgaccacttttgttctcaaactctctcactattat
ctgtggttgccgtttcttcccaaggccagcctggacgaatttggggttgctctatcctgagagttgtaacctcaa
cttccaaagtttatattttcttgaaatgatggatctattgctcaacagtccctgtcatccttaagtgacttctgg
gtttcccacaaattcctcacttttagacacactctaagcttacttctggcctgnatgcttcctctccctgtctct
cccttgcccc
acagcggttccctgacagcagacaaggcagctctgggaggtagctagtatccaataacccaggggtttcctcatg
tgatgcaaatactacgtgtccaaccaatcagtgctgtcaacgggctgccatagctccttcgatggcaaataggat
gtgtgcccaaagaattaaagcgatgagtggctggtgaagcgctctctgtcctggctggttttttgtcaccgcaat
ccactgccaaaggcagaatttaaactaggggctgaaggatagctccgagtaatgtccaatgctggga (SEQ ID
NO: 63) describes presenilin 1 (Psen1), mRNA. Gene name is Psen1
presenilin. NCBI reference sequence is NM_008943.1.
52.
gtccgccgccgcctgctgggccggccgaggatgcagcgcagcgcctcggtggccaggctcgctctgctccagcgt
gcttgctaacttccccggctccgtctctgcctgccggggtcgccccgtgtccctgcgccctccgggtctggttct
ctaagctctctgggcgctgcctccgggtcccttgcagcccgctcgcgagcccgcgctcccctcctgcgccccacc
ctcgtcctcgccatgctgcccttcggcctcgtggccgccctgctgctggccgcagggcctcggccgagcctgggc
gacgaagccatccactgccccccttgctccgaggagaagctggcgcgctgccgcccccccgtgggttgcgaggag
ttggtgcgggagccaggctgcggttgttgcgccacttgcgccctgggcttggggatgccctgcggggtgtacacc
ccacgctgtggctcaggcatgcgctgctaccctccccggggggtggagaagcccctgcgtacattgatgcacggg
caaggcgtgtgcacggagctgtcggaaatcgaagccatccaggaaagcctgcagacctctgacaaggatgagagc
gaacatcccaacaacagcttcaacccctgcagtgcccatgatcacaggtgcctgcagaagcatatggccaaaatt
agagatcggagcaagatgaagatcgtggggacacctcgggaggaaccccgtcctgtgccccagggttcctgccag
agcgagctgcaccgggccctggagcggctggccgcctcacagagccgtacccacgaagacctcttcatcatcccc
attccaaactgtgaccgcaacggcaacttccaccccaaacagtgtcaccccgccctggacggacagcgtggcaag
tgctggtgtg
tggatcggaagacaggggtgaagcttcctgggggtttggaacccaagggggagctggactgccaccagctggctg
acagcttccaagagtgagacctgccagcaggtgggggactcagtgccccctactaggcccctatgctctggggtt
gtagagttgacctggcttggagtctgagtcctggctgtctgcctctggccctgcccaaaatttccctttgtgtgt
gtgtgcacacgcgtgcacatgctcgtgtgtgtgtgtttataggatgtgccctacaggataagcctgagccttctc
gtgcccatgatgggcttagatacccccaagaggtctgagtgtagcaataggaattcccagtgtgctttcaaatca
atatggaattcattcattcattcattcattcatccacctgaaaacacgtattgaaaacatactatgtgccagctt
tagttttcagcgccgggaagcctcgttgtgatttcctccgattgtagaatgtgacgaggtgttctcaactcaagg
cccacgggagaagagaaacctcattcttggagtgagagagaaagaaagtggtgaacattgaacgccctccctctc
catcttggccagagcgtgggaaggtgtagagtagaggctcacggctcctgagacccacctctgctctgtgctgct
gcgtagagctctctccttggggcctccagggcacaagcttgagggacagggcccatctttggtcagggtattcgg
tcatccgacaatgccttgcaggtagatctctgctgtgggaaaggaatggggtgaggaaggccggaaggacagggg
gcagggaggtgggctcatcactctgagcctgcaggagagaggaaagaatgtgagattggattgtgtgtgtctgag
gaatggcccc
attctggtccctatttgtggttattggtcatgaagtcaccaggatgactctatctttctagttgcccaccacctg
tggctttgcctccgtccccatatttccctacccttatacctcctatcccgccatctctacccccctgggcatttt
ctggcttgcctggatagatttagagacctgcttgttggctataatgtcttttcattctttcttcttcctttttta
aaaaaatcagaacaaaaccaaaaaaaaaaaaaaa (SEQ ID NO: 64) describes insulin-like
growth factor binding protein 4 (Igfbp4), mRNA. Gene name is Igfbp4
insulin-like growth factor binding protein 4. NCBI reference sequence is
NM_010517.2.
53.
gagactcagaaagcgccgagagaagcaagatggcggacgtgctggatcttcacgaggcggggggcgaaaatttcg
ccatggatgaggatggggacgaaagcatccacaaactaaaagaaaaagcaaagaaacggaagggccgcggctttg
gctccgaggggtcccgagcgcggatgcgggaggattacgacagtgtggagcaggacggcgatgaacctggaccac
agcgctctgttgaaggttggattctctttgtcactggagtccacgaagaagccactgaagaagatatccatgaca
aattcgctgaatatggggaaataaaaaatattcaccttaatttggacaggcgcacgggatacttgaaggggtata
ctctagttgaatatgaaacatacaaagaggctcaggctgccatggaaggactaaatggtcaagatttgatggggc
agccaatcagtgtggactggtgttttgttcgtggaccaccaaagggcaagaggagaggaggccgaagacgaagca
ggagtccagaccggagacgccgttgataaatcctctgtcgactgggtggtctctctacaagtccatttggttatg
ctgccttggataaacagggctagggcggaccttgtgtttatatttaatttcttaccctatctacttggcattgct
tttgagttttagaaaaaataaatgttccattttgttttctatgtttattactttactgttctggaaactgaacaa
ctacagtggggcattgtgaatgattgtgaagatccttctgtctggcgtggcattctaagtttctagaaaggctca
tggagactgttcagcatttctttgtcttagaagataagggtagggataatgagatttttcttgtggaattaagga
atgtattttg
cataactcttgtgagcagatgttatattagtgtctcttgcttctctgtcagagcctgtgcagctggccttgaact
tcaggggctttgtgatgcttagcctactctatgttgaatatggtgctgaactgtgaagtttattagttcttacaa
tgtaaaagaaacccagcttgatgggctggagagatggctcagtagttaagggcactgactgtccttccagaggtc
ctgagttcaattcccagcacccacatggtggctcaccatgggatcccatgccctctgaagatagctacaacatac
tcatataaataatttttttttttaagaaaaaaaacccagcttgggtactatgtaacaatatgcacacctttaatc
ccagcacttgggaagcaggtggatcttttggtttaaggccaacctggtctacaggagagttttaggatagccagg
gctgcacagagagatcctgtctcaagaaaaaaagacattgctatccctttatcatagggttttggttgttttttt
tttttgggggggggttttgttttttttttttaatttttctgagacagggtttctctgtgtagccctgactgtctc
ccctgtgcctcccaggtgctaggattaaaggcgtgcaccaccactgcccggcttttatcatagtttttagttgaa
attattttttggtatgtctgtagcagctggtttgttgtttgtgtaggtccctatgtgacccacactggcctcaga
ctgatgttcctgtctctctctccccagtgcataatggcaaatattaaacttaacgttttttcagatgc (SEQ
ID NO: 65) describes RNA binding motif protein 8a (Rbm8a), mRNA. Gene name
is Rbm8a RNA binding motif protein 8a. NCBI reference sequence is
NM_025875.1.
54.
aggcaggggctgcaaggaaaggatgccttcggaggcgcagaaccccaaagaagccagctcctgaattctgttact
ccatcagtggcttggaaagtcagatacatcttgagaactctgaaataatatggataaattcatagacaacatgga
tgtccgaattaaatcagaaagtggttcgatgcaggtattcaaacaggtcactggccctgttccgaccagagatcc
ttctgcgagagcagacagaaggaatatgacttctccatcattcctggctgccagcccaatggagaacccagctct
gtttaatgatatcaagatcgagcccccagaagagctcctggagagtgatttcaacatgccccaggtggaacctgt
tgatctctcctttcacaaaccgaaggctccgctccagcctgctagcatgctgcaagctccaatccgtcctcccaa
gccaccgactgcacctcaggccattatggtgcctacttcagctgacacagtcacttcggcagccattcctactgt
tctcactccgggctctatcctggcctcgtctcaggggaccggtggccagccgattttacatgtgatccacaccat
cccctcagtcagtttgccaaataagatgagtggactaaagacaatcccactggtggtccagtctttgcctatggt
ctataccagtctgcctacagatgggagccctgcagccattactgtcccactcattggaggagacggcaaaagtgc
tggatcggtcaaagttgaccccgcctccatgtgcccactggagtttccaagtgatagtgatgagagtgcaattga
aagtggctcttcagccttacagagtctccagggatttcatcatgagccagcgacaatggtccacatgcagggaga
agagtctctt
gacttaaagagaagacggattcatcagtgtgactttgcaggatgcagcaaagtgtacaccaaaagctctcacctg
aaagctcaccgcagaatccatacaggagagaagccttacaagtgcacctgggatggctgttcctggaaatttgct
cgctcggatgagcttactcgccatttccgtaagcacacgggcatcaagccattccggtgcacagactgcaaccgc
agtttttctcgatctgaccacttgtccctccaccgccggcgtcatgacaccatgtgaatatcacaggccacagca
ggcacgctgcaatggcctcctgagtgtgtgctgaagttgtaactacgtttgcctctttgactacaacttgccccc
atgattggggtccgaacagcctactgagccaggttaatgagactggaaaacaagttagctggtctcccatgcggc
tcttcatattccactttcacctcttcactgtccagacttcagtttttgcaacctcttcctgggttgaattcagtg
cggcgcccatggctgccctccctgatccctcatgctacattatagacatttttcctacaataacaaacaaaacag
gaatcaaactcaaggctgtgaacaaacatccgctgctctcccctcccccctggtttctcttgttttctaaagatt
ttattcacatgtgtgtatatatatatacacatatatgtgtatgtatatatatatatatatatatatactgtaacc
agtacttaatatcacttagcaatgtcctaaaatgagggttttttttcaaagttgtagctacaaggggcataacag
tagagttggttgatcaaatttacatagaattgagtctgaaatacagagtctcatgctaaccgtgaaggttttgta
gatccagttt
tacttgttcttaacagtttggtttacaggcagtgccagccctccttttgaagttcatcaagagcaatgaagggat
tccttgacctccttttcctcctcatgctcctttagaataaaaggttttatggcacaattccattctttttaggtt
aagtttggacaaatattcaaagcacagaaagatagatcagtagggagaagtattctaggaaaatacgtgggcagt
gacaggcaagagcagacctgggagcaccctgtgaacaattacttggataaggtgttagaactgctgttaatccca
agcctcgttcagagggcagcctgttgaaacatcactttcgatgggctccactgtactgttgattcttttgtttac
ataaggcaaccattcagcgtattacctgatgagaaattgcacactgagcacgttatagagaggtgaggggttgca
ctaaaggaattttgtttacttcatgccattttgggcatttgttcttctctttctaatgggatggaagaggttttc
tttctgacactgacatatagaaagcatttttttggatgttttacaacttctattaatacccaagccatttatttt
atttagatataacaacaagatgcgtaatatacatttggttgcccaatcagactgttcataatattctctggggca
taaaaccttctgttcctggccctggacctcatatctacctcttcgtggcatatttcctctcctggggaaaaagga
gaggggctttgtattagtaggattggacacagcctctcgcctaaaggttttcttcatatgaaacctttatttctt
ggcagatattcaaaatgttctggcttgaacaatttaggttactacctcctgtggctgtactttctaactaaagcc
tagaattcta
ccatccagaaagggtcctgtctacttctacccatccctcacgttttacagatgaggcaaatacgggtcagattct
ttctccagataattccttgaggccttacagacctgtatacaccatctcccagggatcacactagtaaaccaaatt
aaagaaaaagaagttatgtgattggttgcaatatttgtccaattttatgttacacttcagaaaactttaaattca
tgtatatttggtgacaaagtaagtagggtgtgcttatggtcatgaccacttaacaccaggcacacagtgtttcaa
gtcacattttttgataattctagatttgttttaatttaccttgtttcaagtttcaatgtctttgcagagcataaa
ttctccataagttaaggcctacttgtacttttcaacgtggcatgtaggttttactgttactgctttggtaaggga
tctgtcctgactctgggtgtacaagttccaggctgtatactaattactaagcactaatcaatatctagggctagt
ctactaatggggaccacaatgtaaaacagattgaaaaatgtatattcctaccatatatgggcttaagcttataat
tgagtagttggtacaatactatagaacagagtccaaagggttatgtacttctgttccactttggatcctaaattt
gttttccagagattggaattggcatgtgccccttagacttcaggatattgagacatatgtttattaatgagcact
atgaaattatagtactcataagggtgatcagctgaggatcccagatactgatgttcctagcatctctgagccttt
attcaagtatgtttctgcagagtcttaatagactttgcaaatgctttgtccattgtctttagccaagaaccaatt
ctaaccatat
tctctttgctctttttgttcccaaatggcaaaaatctttccagttacaagcatagtctttacaaatgtgtcatga
ttaagagaacttcgcagagtaaatgaactcaaacagtacagtgcagaattactgcaccaaaagcacatacgtttg
ctacatagtgggatataaaggaaaccattccacttctcttggttaacatctgaggtttaactaagtgtaatgggc
ttacaacagccaaagactgttaggatgacatttgcctaggatatggaaattgtgtctggatggataatattcagg
agtttattgtagtgaagtttctccctctgtagacatctggcagctgaacataattgttggtaattgccattgtaa
agtgtaagaaatgtaaactggtttgatggattagtggagttcattatgtacaaacccacgaatgtttgtaaagag
aatttctttttaaataaactttacatgttggaactctttttttgagattttcaggaaaatgtgaaggtagaacag
agttcctgctgactctacattatatatttgtttggtatacttgttacaattagtgaaattataccgatacattaa
tattaactacaatcaatgctttcgtcagactaccttggcttttatctgctgttctttttttctgttttaataccg
ccttcaaaatacaacattgtattaataataacagctccttattctcctcgttgtgatagcttcttggagttcttt
gttctcaaggaccttggagtatgtttttcagttggggtgtattttatattcttctcatgaaaattttccc (SEQ
ID NO: 66) describes Kruppel-like factor 8 (Klf8), mRNA. Gene name is Klf8
Kruppel-like factor 8. NCBI reference sequence is NM_173780.2.
55.
gacggttcggcctggtgccgcgagcggccgttgccagggtgggggtcgctttgcggcatggcgatggcggagggc
gagcggactgagtgtgctgagaccccccgggacgaacccccggccgatggcgctctgaagcgggcagaggagctc
aagacacaggccaacgactacttcaaagccaaggactacgagaacgcgatcaagttctacagtcaggccatcgag
ttgaaccccggcaatgccatctactatggcaaccgcagcctggcctacctgcgcactgagtgctatggctatgca
ctgggcgacgccacacgggccatcgagcttgacaagaagtacatcaaaggctactaccgccgggcggccagcaac
atggcactgggcaagttccgggctgccctgcgtgactacgagacggtggtgaaagtgaagcctaatgacaaggat
gccaagatgaagtaccaggagtgcagcaagattgtgaagcagaaggcctttgagagggccattgcaggtgacgag
cacagacgctctgtcgtggactctctggacattgaaagcatgaccattgaagatgagtacagcgggcccaagctt
gaggatggcaaagtgacaatcaccttcatgaaagacctcatgcagtggtacaaggatcagaagaaactgcaccgg
aagtgcgcctaccagatcctagtacaggtgaaagaagtcctctgcaagctgagcacgctggtggagacgacgctg
aaagagacagagaagattacagtgtgcggggacacccatggccagttctacgacctcctcaacatatttgagctc
aacggtttaccctcagagaccaacccctatatatttaatggcgattttgtggaccgtggttccttctccgttgaa
gtgatcctca
ccctcttcggctttaagctcctgtatccagatcatttccatctacttcgaggcaaccacgagacagacaacatga
accagatctacgggttcgagggcgaggtgaaggccaagtacacagcccagatgtatgagctcttcagcgaggtgt
tcgagtggcttccgctggcgcagtgtatcaatggcaaagtgctgatcatgcacggaggcctattcagcgaagatg
gtgtcactctggatgacatccgaaagattgagcggaatcggcagcccccagactcaggtcccatgtgtgacctgc
tgtggtcagatccccagccacagaatgggcgctccgtcagcaagcgtggtgtgagttgccagtttgggcctgatg
tcaccaaggccttcctggaggagaatcaactggactatatcatccgcagccatgaagtcaaagccgagggctacg
aggtggcccatggtggccgctgtgtcactgtcttttctgcccccaactattgtgaccagatgggaaacaaagcct
cctacatccacctccagggctccgacctgcggccccagttccaccaattcacagcagtgcctcaccccaatgtca
agcccatggcatacgccaacacgcttctgcagctaggaatgatgtgaggctgtcagcagcagtgagccgggactt
gcccagctgaatggccccgccctcggcctgctgccccgccccagggcaacgttggacccccttttactttgtaaa
gtttgtatttattcccctttaggttgcagagggggtgggggcagcgtggggcagcagacgacaggctcctggtca
gagaaaggagactggaggtagggcacagcctgggcatttggaggaaggccagcctgggtaggggtggggccaaga
ggaggtcttg
ccccttcacctgcatggctcctcccctggtaaagcaataggaccctgccataggaagaccccaaaagagggtcat
cagggaggcctcctcctggtagcctcagggtggggcaaggctggggcccaccccccagctattttatgttctgta
attaactatgttaaaataaaataaagtcattattgtaagtcaaaaaaaaaaaaaaaaaaa (SEQ ID NO:
67) describes protein phosphatase 5, catalytic subunit (Ppp5c), mRNA.
Gene name is Ppp5c protein phosphatase 5, catalytic subunit. NCBI
reference sequence is NM_011155.1.
56.
gcgtcgtgcttgcgggctggagcggttgctgccgtgagtccgagtgctttcgcgagccccgtcggcttcccggtt
cggtcggtcctcgacctcccgacaggctgctcccaggctgcgtctgggctggaaggtaagacttatttgtagtag
ataaatagccaccatggagaccaaggaccacaagaaacataggaagaagcacagtgggcctaaagctgagaagaa
aaagaaacggcacctgcaagacctacagcttggagatgaagaggatgcccggaagagaaaccccaaagctttcgc
agtgcagtcagctgtgcggatggcgaggtcctttcacaggactcaggacttaaagacgaaaaagcatcacattcc
agtggttgacagaactccactggagcccccaccaatagtggtagtggtgatgggacctccaaaagttggaaagag
cactttgatacgatgcctgattcggaattttaccagacagaaattgagtgagatcagaggccctgtgacaattgt
gtcaggtaaaaagcgcagacttacaattattgagtgtgggtgtgacatcaatgtaatgattgatctagctaaagt
agcagatctggtgctaatgcttatagatgccagctttgggtttgagatggagacatttgagtttctaaacatctg
tcaagtacatggctttcctaaaattatgggagttctcacccatttggactccttcaagcataacaagcaacttaa
gaagacaaagaagcgtttaaagcacagattctggacagaagtctacccgggcgccaagctattctacctttctgg
aatggtgcatggagagtatcagaaccaagagattcacaacctgggccggtttattacagttatgaagtttaggcc
tctcacatgg
cagacttctcatccttacatcttggcagacaggatggaagacttgacaaatcctgaggctatccgcacaaacgtc
aagtgtgaccgaaaggtgtctctttatggttatttacgaggggcctacctgaaaaataacagccagatccacatg
ccaggcgtgggggattttgtcgccagtgatgtcagttttctcccagacccctgcgctcttccggaacagcagaag
aagcgctgtttgaatgagaaggagaaactggtttacgcgcctctctctggggttgggggtgtgctgtacgacaaa
gatgctgtctatgtcgacctaggtggcagccatggcttccaggcagcggaagaaacaggccctacgcatgaactg
gtccagagcctcatctccacccatgccagtatcgatgctaagatggcttcaagtagagtgacacttttctctgat
tccaaacctctggggtcagaagacatagataatcaagggctgtggatgccgaaagaggaaaagcaggtggatgtg
gaaacgggccgagtgcgtcggaaagccatttttggagatacagaagatgagtctggagatgaagagagtgaagat
gaagaagacatgtctgaagctgacgggatggaacctggctctagtgatgatgaggcggaagaggcagaggaagaa
ggtgctgagagcagcagcggtaactacatgatgggtaggggacgcaaacgaccaaagcttgaggagcaggaggaa
gacagtgaggcagactcgccagcgtttgctgatagcgatgatgaccttgagaggagctctgggggcgaggaggag
gccgaggaggcggatgaaagcagtgctgacggtgcctccacaggggagagggacgctggggaatgcggctctgtt
ggagaagcta
gtgaggagagcccgtcagcagcccaccagtggaggccccatgcggatgcacagaagccctccccggtgagaaaag
cagccctcaccacttctgactctggccactgtacagccgaggaggcgtttccatctgaggatgagtctgaggact
ctgagctcagtgcagaggaagaagactccgaccacagagaggttagggggaagcttgcacaccctctgcgcgaca
gcggacagaaacctgcctcggagagcctggttgatgagaccagcgacattgaaagtttactcaaagaggaggaag
agtacaaggaggacaatagcagctctgtagaaacgtcaggtgctctgaagtggaaggaagacctttccagaaaag
cagcagaggcctttctcaggcagcagcaagcagctccgaatcttcgaaagctgatttatgggacagtgacagagg
ataatgaagatgaagatggagaccccgcagaagagcttggagggctgtttcatgtcagccagccagatagagggt
gcaagcacaaggctgactctctggactgctccagattccctgtggaggctcctcacgactgggatttagaggagg
ttatgaacagtatcagagactgctttgtgactgggaaatgggaagatgataaagatgcagccaagatcttagcag
aggaagaggagctctatggtgactttgaagacctagaaacgggggacgtgcacaaaggaaaaccaggcccggata
ctcagagtgaagacatagaggaagaagaagtgaaggaagaaactgacccaagtgaggaggaaagtgctaggaaaa
agcatctggataagaagagaaagctgaaggagctgtttgatgcagagtatgatgaaggagaaagcacctactttg
atgatcttaa
gggggaaatgcagagacaagcgcagcttaaccaggctgaatttgaagatcaagaagatgaaaccagagttcagta
tgaggggtttcgacctggaatgtatgttcgaattgagatcgaaaacatcccttgtgaatttgtgcagaactttga
cccacactacccaattattctgggtggtttgggcaatagcgagggcactgtggggtatgtgcagatgcgcatcaa
gaaacatcgttggtataagaaaatcctcaagtctcgagaccccattattttttctgtaggatggaggagatttca
gaccatcccattgtattacattgaagaccacaatggaagacaaaggcttctaaaatacaccccacaacacatgca
ttgtggagcaactttttggggtcctatcactccacaaggaactggatttttggcgatacagtctgtcagtggtgt
aatgcctgagtttcggattgctgccactggagttgtccttgacctggataaatccataaaaattgtgaagaaatt
aaaactaactggttttccttttaaaatattcaagaatacttcatttattaagggaatgtttaattctgcattgga
agtagccaaatttgaaggtgctgtcattcgaactgtcagtggaataaggggacagatcaagaaagcactccgggc
tcccgaaggagccttccgcgctagctttgaggacaagttgctaatgagtgatattgttttcatgcgaacttggta
ccctgtctccattccggccttctacaatccagtaacatctttgttgaaaccagtgggtgagaaggatacctggtc
agggatgcggacaacacatcagctgaggcttgcccatggaatcaagttaaaggcaaacaaggattccttgtataa
gccaatcctg
agacaaaagaaacattttaattcactgcacattcccaaagccttgcagaaggctctgccatttaagaacaaaccc
aagactcaagcaaaggcaggcaaagtgccgagggacaggcagagaccggccgtcatacgtgaacctcatgagagg
aaggtcctggctctgctagatgctctgagcacaatacacagtcagaaaatgaaaaaggccaaagagcagagacgc
ctgcacaacaaggagcatgtgaagatgaagcagaaggaggaagaggacaagctgaggcggcagaaggacctgagg
aagaagctcttccggatgcagggtcagaaggaaaaacgaagtcagaagtctagtctgagaggagctgaggatacc
tgaatggagggggggtcttccagagctgcagccgtgatggcttcagtgcttgtgacaagtcatgacgagagcttg
tggagactctgggatgtgtggtcccgccgtgcctggaggaactgtagaaagcctgctggtggcatccgtgctcat
tcacggcatccagcaagtcacagttcaaggctgggaaacttggggctttgtgaattcactcattggttcttcctg
acttggtttattttgtgctataaaaatattaaactattttcccattaaaaaaaaaaaaaaaaaaa (SEQ ID
NO: 68) describes BMS1 homolog, ribosome assembly protein (yeast) (Bms1),
mRNA. Gene name is Bms1 BMS1 homolog, ribosome assembly protein (yeast).
NCBI reference sequence is NM_194339.1.
57.
caagactggtcagcggctccctaaccccgggctcgcaggctgatgaccctgggagacaggggcctcgaggggccg
caaactagcgcgccgcgggctgagcccgagaggccaggctggggggagggaggcccggagggagggagtgaagga
aggaggagggctggcgagctggagccggcaggcggcgggagcccgggcgagcctcgtcgggagtgcgtctcctcg
gcagagcaggacgcagccagagcttgtccaggtgactgagctccacccctgtgcaccaagacacccaccccaccc
cccactggtcaagcctcgctggagaaagagaccgggaagtcccagcatgagaatcagagctgcccagccagcttc
atagatggggaagcagcctgcttgcctcccccatcaattacagggataactgagaaaaagccccacccaatatgg
aggctgaggactaccgcctctattaattccctgccttctcccctgccctgcccacctggccttagctgggtcact
catgcaatgctgagaagtaagcaagacttggggtcagcttgcctggctaccaggcaaggactgtgggcatcatgg
gggtgtgtgtgagcggggctccagggtgagacctagcccccgcccccaggaattcaagggggtgggggtggggcc
gaggatagaatggctcggggagggttgggggcagaagaggcctccctacgctcaaacgcattgtcctggctggcc
tgtgggctcctggccctgctggccaacgcttggatcatccttagcatctcagccaaacagcagaaacacaagcca
ctggagttactgctctgcttcctggccggtactcatatactcatggcggctgtgccccttaccacctttgctgtg
gtgcagctcc
ggcgccaggcttcctctgactatgactggaacgagagcatctgcaaggtctttgtgtccacctactacacgctgg
ccctggccacctgcttcacggtcgcctcgctctcctaccatcgcatgtggatggtgcgatggcccgttaactacc
gcctcagcaacgccaagaagcaggcgctgcatgccgtcatgggcatctggatggtcagcttcatcctctccacgc
tgccctccatcggctggcacaacaacggggagcgctactatgcccgaggctgccagttcatagtctccaagatcg
gcctcggttttggtgtctgcttcagcctcctgcttcttgggggcattgtcatggggttggtctgtgtggctatca
cattctatcagacactgtgggcccggccccggagggctcggcaggcccggagagcggggggcagtgtgggcacca
aagcaggggggctgggggggttgggtacccggccagcttttgaggtgccagccattgtggtggaggatactcgag
ggaagcggcggtcctcgctggatggctctgaatctgccaagacatccttgcaggtcaccaacttggtcagcgcca
tcgtctttctctatgactcactcacgggggtgcccattttggtggtgagcttcttctccttgaagtcagactcag
ctccgccatggatggtgttggcagtgctgtggtgctccatggcacagacactgctcctgccctccttcatctggt
cctgtgagcgctaccgtgcagatgtgcgcacagtgtgggagcagtgtgtggcgatcatgtccgaggatgacggtg
atgacgatggcacctgtgacgactacacagatggccgagtgtgcaaaattcgctttgatgctaacggggccacag
ggtcagggag
tcgggaccccacccaggtgaagctgctgcctgggcggcacatgctcttcccaccgctggagagagtacactactt
acaggtccctttgtcccgacgattatcccacgatgagaccaacatcttttctactcctcgggcaccaggctcctt
cctgcacaagtggtcctcatcggatgatatccggatcctcccagcccagagccgagccctcggaggccctccaga
atacctgggacagagacacaggctggaggatgaggaggatgaggaagaggctgaaggcgggggcttggccagcct
tcgacagtttctagaaagtggggttctggggtcgggtgggggacccccacgaggtcccggttttttccgggagga
gatcaccaccttcatcgatgagacacctctgccttctccaactgcgtcaccaggaccctctcctcgtaggcctag
accactgggtttttcacctcgacggctctcccttgggtctccggatagccgagccgttggacttcctttggggtt
aagtgcaggaagacgctgttccctgaccgggagcgaagggagttcaagggcttggggaagaccctgggggccagg
taaccccatcttcccccagttgaccctgtgaggccgcataggcctgcttcactggaggggggggggaggcacgag
cgagtgagtatgcctcatcctggccctgagtctatggcagctgcctccagggagatgggggggggggggcctaca
tccgggctctagagccccagctctctcccatccaagtgaccagatacccaacgcacccgtcttcacccctagcaa
aatgtattaaaaggctgagctgccgtggaaaaaaaaaaaaaaaaaaaaaa (SEQ ID NO: 69) de-
scribes G protein-coupled receptor 162 (Gpr162), mRNA. Gene name is Gpr162
G protein-coupled receptor 162. NCBI reference sequence is NM_013533.2.
58.
agcatggagggtctgtacttgagtctaactaggtcagatgctggggatctttaaagctgattcctgcaataatgc
caggcagtctggttttatgacaaggttttgacctggacttctggaccagatgcctgggaacagatagtgtcaagc
ctttctgaatctgtcgcagatcatttatgaaaattgattttagccaatcataaattgtttgctactgccttccaa
ggtaagtgaaaaacgattggactgagtctatgttatttgacctcaggtgatattcccaaagagaaatgacagaag
cttatctttgaatgcaaacaagaaatgtgaacctcctgatcagtgcagcactgagtgtgttactaatctatgtat
ccctaccccttccctctctctcttttaccctcctccctcctctcttctccctcttctatcctaccttcactttca
agaaactagagagccacctttttataagaaagttaagtgtcttaaaatttttaggtagaaaattttgtacgtttt
catccgtgtggctataaacaccaacataggactctatttgcatattaaaacaaacactgcagacactgatctaac
cttccgcagtgttcaactggaatttgttcagatgtcagccatgatgctgattggtaatgtatcagtggaaaacat
taaggaaattggggtgtgacctaatggacagtaattacaaagctattacaaatctcattgcataagtaatgtctg
gaaactttgtgctctctcaaataaacctcgacagccagacgagtgcttcgcaatcacacagtggcatcataatcc
cgaaattgactgttctgtgctaattcacgctgatgacctcttttaaaattatttttaatttggcttcataagaac
aattccaaag
tagaagtgtgttgtcttttctggcattgtttttctttttccaatttgaaatgcaaccagctgtttctgtgtaata
aaaactgaaccatgaaatacaatggctctttcaagcttcaacctgcaggcagctcacaattagcaaccttgtgtt
atactggaaatagccaattaaaaaaatgctgaacattctacattacaaaatatcaaatggcatgcaatcaaggtc
cctttaaaaggcaagctgctctttctctataaatttgccaataaaacgattaggatattattctaaaatcagtat
gtaaaaatttccaaaagtcttttttaaaaaatctgtcagtgtcagtgttgaccagttccttctatgaggcttcac
aaatgccagacatgatggcatttccattttgtgggtatctgccatatcaccaccttcaactaacatatccaccat
gcaactgtattttccaggaggcttctggaaagattgcctgcatacatgaggatgatgtccagctgagttcagtgt
ctagagaatggactctgtatataggctgctcagtggtgacacgcggtgggcccacaataatttctgctaagtggg
aattctgttggctctttggcagtactcaggaaactggggtacagtcaggtttccagagccaaatggggataagct
ggactctgaccagtacatacactttgacaatgtgtaaggaagtgttactttgatgtgataacacagatcactttt
ttttatttaagaaaatgcatgtccaggggtagccaggatttatgcatctacacctataacatttgcatcttagct
gctcaccaactcaccaatgtggaaaactaagtcagctcagatctgaccatagaaactcaagtcttactctgctcc
gaagaaaagt
gctctaggctagactagcatgtctaccataggaaccactggcttaatgtggctcatgaccgcccgatgtaaacat
gcttaaactgttaatttagaaacagtataaaatagattcaaattatcttattaattttaattttgatgtaataat
atgtgtatactggattaaaatatagtattaattccatattttctttttagaatgtagctaatagaaaatgtaaag
atacatgcatggttatgggagtggtggcatatgcttataaccccagatccagggaaagatgaggcaggaagatca
ctgtgtgagtctgaggtcagcctagattacaaagtgaattcaaggccagcctgcaattcataataaaaccttgtc
tgagaattgtttttacatgtgtaccttattatactttttaatataaacacttatttcagaagataactttgtaac
cattctattctaatctctcatttatgtgctgttgtggcaagagggctaacagtttgagaaaccactgtaagcaca
ggtggactttctaaataggagaaaaaaatttgcagactgtcctctgtggtgaaagaaccccgttaagctatatgt
ctgatcttgaaataaaaaccaataatgaatgg (SEQ ID NO: 70) describes 10 days neonate
cortex cDNA, RIKEN full-length enriched library, clone: A830007O09
product: inferred: calcium/calmodulin-dependent protein kinase IV, full
insert sequence. Gene name is LOC328931 hypothetical gene supported by
AK043558. NCBI reference sequence is AK043558.1.
59.
gagtctgagttttcccgttcaggtactatactagtccaccagtgaccccggacagtaacaaagcattacagcccg
aacccacctcccgccaccatcgtaggttctgtacctcacttgggaatcagtaaaccatggcatctaagaaatttg
ccgttaaatgtgggaacttcgcagtcctggtggatcttcatgtattgccacaaggttcaaacagagactccagtt
ggttttctgaacagaagaaagaggaagtttgtttgctgttaaaagaaaccattgattccagagttaaggagtatg
tgggcatttataaacagcgcaagccatcaagtgcagaattcacaagatccagtcccttgtccttaaaaggttatg
gctttcagatcacagcttatttcctcaagagagggatacaccttcactgtatccagaactcccagaatactgaac
ttcgtgtatttcctgaaagatttgtggtgtgtgttagtcagcttgcatttggtcatgatatttgggcaaaccaga
atgaaaaatcgacaaaaaaagccctccatggagtgtctgattattttcctgagtgtgcagagagttcaccgtctc
ctggtacaaagctcaagaggaacgctctgaaggaaattgtgagaagaactaaatcaaaaggcactgatgtgagca
aaccacagcccagcggggacctcgtgggaagatctagtgactcagtaattactgtggtaccatggaggagggatg
cctcagccattctcctgtcagaatctgtgggtcaagcacaagatgacataagagcagccaagagccaccaggagc
ttcctgttcaaaagctggaaaatgtttctcagactcagccaggagacactcgcagccagcagcaacttcatcctg
gggagtggtt
gaagacggggcttctaagcaggagccctgcgtataactatgagtcagcatcaccaggtccaaagcaaagtctgag
agcagccaagacacagcagaaacacaggaattgtggctctgtggaagactgtgaccaccgcaggagagtttctct
tggaaatgaaggattagtcccggaggacgcggatagggaaaggagcacagctgtcagggttttaccagccttaga
gttgtcagatcctgggttgcttttgaaacaagatttggcaaaagccaaggctaaggaagagctacatgctttgga
aaatctctcctccagacatcttgtgacaaacaacccggggcaggcacagcagagcgactcagctgcaattactga
acaattagctacagatcagggcggccccagcaagaaaagaaagaaactacaaagttacaacagaggatgtagtgg
caagaaaaactgaggatacagtacctggggtattggaaggaattaggacaattactttaaataaataaatttagg
agc (SEQ ID NO: 71) describes RIKEN cDNA 2210009G21 gene (2210009G21Rik),
transcript variant 1, mRNA. Gene name is RIKEN cDNA 2210009G21 gene. NCBI
reference sequence is NM_028834.1.
60.
tcctgggagaatccgtcaccgggaaagagtagaaaccacctggtcgaggcgcagaagtggctccggagggagccg
gcgtaaggtctgctgcctcgtgtccgctaagggcgcggcgatccgggatccgccggccggagcccaagcgcagcc
gggagcttgcgggtggcgaagccaggcgcaccgccttctgggtgtctgctgagcggttccccgcgtcccgcgggg
tcctgcagccccgaccccccggctggaagccacggagaaccgctgccgaggactgcggaccaacatcgtccctcc
cgaaagcggattctgctatgacagctgggctctccaaggggttaacctgggtgtcctccgtaaagttgtcgccga
gccgggagcccgtttaggggtcgcagcgccgcggctcggggggcggccgagtggccggcggcggccgtggctcgg
cggaacccgcgcggccggggggcttctgggggtgtgcgcccccagccggctgccctcgtggatgcctcccggcgg
cggcgggcccatgaaagactgcgagtacagtcagatcagcacccccagctcctctcccatggaggcgccccacca
gaaaaagaatatcgctgcccggaggaaatgggaggtgttcccaggaagaaacaagttcttttgncaacgggagga
tcatgatggcccggcagaccggcgtcttctacctgactctcattctcatcctggtcactagtggactcttcttcg
ccttcgagcagctgtcggtacctggcagagaagatcacccctgccatccccgtggtcggtggcatcctgttcttc
tttgtgatggggacactgcttcggaccagcttcagtgacccgggagtcctccctcgagccactcctgatgaagcc
gctgatctgg
aaaggcaaatagatatagccaacggcaccagttcaggggggtatcgcccgcctcccagaaccaaagaagtcgtca
tcaatggccagaccgtgaaactaaagtactgttttacctgcaagattttccgcccccctcgagcctcccactgca
gcctgtgtgataactgcgtagaacagtttgatcaccactgtccctgggtaggcaactgtgtggggaaaagaaact
acagatttttttatatgtttattttatccctgtcctttctgacagtctttatatttgcattcgttatcacacacg
tcatccatcgttcacagcaaaagggattcctcgatgcccttaaggacagtccagcgagtgtgctagaggctgtaa
tatgcttcttctctgtctggtccatcattggcctttcaggtttccacacgtgcctgatcagctccaaccagacca
caaacgaagacatcaaaggctcttggtccaataaaagaggtaaagaaaattacaatccctacagctatggaaaca
tcttcacaaattgctgtgtcgccctatgtgggcccatctctcccagcctaattgacagaagaggctatgtccagc
ccgacacaccacagccagcagcaccatcaaatgggatcactatgtatggggccacacagtcgcagagtgacatgt
gcgaccaagaccagtgcattcagagcaccaaattcgttttgcaggccgcagccacgcccctgcttcagagcgagc
ccagcctcaccagcgaagagctgcatatgcctgggaaacccggcctgggcaccccatgtgctagcctgacgctgg
gccagcccacaccaccctcctccatgcccaaccttgccacggaggccacactctcagacattatgcccctgaagg
atgagcatgg
gggccatcagtttctgaccccagatgaggcaccctcaccgccaaggatgctgggggcaggcagccctctagcaca
tagccgtaccatgcacatgttgggtctggccagccaggactccctgcacgaagactccgtgaggggvttggtaaa
gctcagctccgtgtgacctgcatggccagccctggggaccacaggtgacaaacaaaaacaaattactggacatag
ccaagaggacgttttgtttggaatctgcagagtggaccgggaagaggagactccctcaatgggaggagcctggaa
ttgaaagaggcagcgtggatgcagcgtgcagcgccctctgtcggctgcttttgggacagacacgaggaaaatttt
cgacactggtggccagactgaaattggacctaatgttacagctactaattgaaatccacccgccgttccatttgt
tcattaaaacaaaacgaaacaaaagttccaaagggaaaagccggccagacatgggattgcatgccacactatagt
ctgtgttacagcagtggtattccagaagaaaagatgtctctctgttttccttctttttaatcttgtgatactttc
caatgtgacagaccgaacccggtgggtaatgccatccttcgcgacttactggacttagctgtatgaggatgggcg
tttcctgatgaagagggttactgggggcatcccctgttgcagacggcacctggggtctatcctgccctggacaga
cattttaaccctctgtcaatgctcagatggatgtcatattgtgccaccatggggcagtcctcgcctcctgtgtga
caatgaccttcctatggacagccaataaaatgactcctctgttaaaaaaaaaaaa (SEQ ID NO: 72)
describes zinc finger, DHHC domain containing 14 (Zdhhc14), mRNA. Gene name
is Zdhhc14 zinc finger, DHHC domain containing 14. NCBI reference sequence
is NM_146073.2.
61.
tagacttcggccgtggtggccaggcgctccccacacattgtatacgcttgcagcttcaggaagctgctcc
tcaggctgtgcccatcctgtttggcagcccagctaccatggaggccccagaagtccctgtgggctccctg
attgactttgagactgaaccacctacctcccctcctttggaagcagcaccgtcaacgctccaggacccag
atggctccctgggtgatggtgcatctgagagtgagaccaccgagtccgcagacagtgagaacgacatggg
cgagtcaccttcacacccatcctgggaccaggaccgacgctcttcctccaatgagtccttctcctccaat
cagagtgctgattccgccccagatgaggagaccctggcacttcgagagtttatgcgcagctacgtagaga
agatcttctctggaggggaggacttggaccaggaagagaaagccaagtttggggagtactgcagcggaga
agacgggaaaggccgggaatggttcgcacgctttgtgagcgcacagcgctgcaagtccaagtgtgtgtcg
gagccaaccttgtaccgcctggtgcagtccttcgcggtagtgctgtttgaatgccaccagatggacgact
tcgggcctgccaagaacctcatgaccatgtgcttcacttactaccatctgggcaagccccagctgccccc
gacagagcccagggagaagccagcaggcagtattgactcctacctgaagtcagccaacagctggctggca
gagaagaaggacattgccgagcggctgctgaagaacacagagaacatgaaaggctttttcggggggctgg
agaccaagctgaaaggaccgctggtcaggaaaaatgaggaagatgagaacaaacctaaggacagacagac
gaagactgtgaccatgatcagccctgaggatgagcagaagggggagaaggtttacctgtacacgcacctg
aggcagcagcccatctggcacacgctgaggttctggaatgctgccttttttgatgctgtccattgtgaga
ggcggaagcgatcccccaccaccagaggggatgctggagagcaggaggagaagagggagaaatggtgcca
catgacccaggaagaacgggatgacagcctgcgcttcaacgagaacatcaccttcgggcagctgggcacg
ttcacccacaacatgctggcctttggcttgaacaagaagctatgcagcgacttcctgaagaagcaggctg
tgattggcaatctagatgaagagcaatacaagctgctaagtgaccacatcgagcaaatggccacagagta
ggagatcagcacgtgctcgtctcctaggatctgtcaccccagtgatgaccctgcatgacaccagcagcac
ccagagccgctccttgctgccctagaactagcagttaaaagactccagaagcctgtctcccctgcctgtg
tctgctctccctctatgtctatggtgtcccctgggttacacagctaaacgtgagggaccatctacccccc
acctatcctcgagcaccaggcttgtgagatgagacagagggtggaggaagccatgagggatggtccccac
ggatcctgccgagagccacggacatgtggttacagaggcgcaggaggggatccacacagctgcaacgtga
ggacagaatcagtgctgtgaatgacagcctgccactccctgccacaggactccctgtgagctgtacagag
gaccatggagccttcctgaaaggtctcggccatctgttcttctccacctcctcatgggataggaactgaa
agagaactcaagggttgtcactttcctagagggtgtgagcgttctgccagccacaaccatgtctcagact
caagagctccgtcaacagagcctcggaactggccactcaatacagtctctccctcaatagcacagaaggt
ccttgagctcccaggccgcagcaacacatgcagtgggacgcaggaagcgtgggcgagcattctcctcggc
agctctgggggggacagagctaagagcacacagaatggatctgactgagggaggagaccacgggaaagca
aggttgcctggtagctccagcccacagtcctccccttctgaaagacccacagcgatctgcctcctcagca
tccagcgtgctggctccagagctgctgttcccatccacgccagtggcctcggtcccttctgtgccaggca
ttctggtcactgaccttgaactaggcctgcaggctctcctgtcaccccgcatcacctcacccttctctgt
ccctcagccttcggtcacatttagtcatggtctgcctgtgagcgcagtgagttctctcctgacagctgtg
agctcagctggtgtgggtgagcctttcctgcaggaagagcctcccaagggatggccaaagaagagcagga
taggcaaagctgtggaatgtacatttcctggatgtcaggggtgaccaaaacagtacatcagttcctgaga
tattcaagcaaatgcccccgatagcatagtgtgtgccaaccggtacacctgactcagcattaggatccta
taaccatgcctgtccctgctctgtgttttagggaagtgacataaagtgaggtcgcccttgctaagtcctg
ctgtcctctctagagccttcctcagagctgtgcaagcatgggagcctctccttgctcctcccagtcctgc
actggcatgagttcgggtgtaggaggacatgactccaaggtgctgggagatgcaccagtcttgcgcagca
gcacctgtggtctggcctctgccttgccctcatgcagccatgagctgccttaaggggaagagtgccaatg
gtcttcgtgggcttcagaagtgactttgagactgttcctgctgaccttttgagatagtcacttaaggaag
gatggctctctctcatctgtctcttgttccttattgttgcagagctagctccacagagtcagaaagttct
gacccacattagacccgtgtgcgctgctaaggccatggtaaccctctggggacaggcagccattgatggg
agcaaagcttgcaggtgcctggaggtggtcccaccattgcctgcagatgtccttcctctgttcacagtta
ggtttagtgagcatttcatgtcagggagcctctggccagtgtctctgggaggtcactaatctacccctga
ggtacccactctttctgtcgtagctttcttctagggccccaacaagcaagcataaaccaggcttgctggc
cagcactccttggctcccctgccgaaccacagacagacagttcagattaccaggataatcccaagcagta
tctccagtctcggctggtagtggcccagatctatgcaggcccattagcacacattcaaaccttccggggc
tgtgaccttcaaagctgtgtaaatcagtgtcaagcacattatagtgtttgccctc (SEQ ID NO:
73) describes RIKEN cDNA 6430548M08 gene (6430548M08Rik), mRNA. Gene
name is RIKEN cDNA 6430548M08 gene. NCBI reference sequence is
NM_172286.2.
62.
gcggccgcgaattcggcacgaggggcggcgcgaggccggccgggagggaacagcctgctttctccggagt
ccggagcagccaaggactgagctgagcaagcggagcgcggtgcctgcgggaagccgggacagcagcagcc
tcaccgtgccatgcgggcctcaggattgtgccaatgaggacctaaggtgcctgcccgccaaccatcccag
cctgcagtgaccatggccctccgcaagaggagtcctcatggcctgggcttcctttgctgctttggaggca
gtgaccttcccgagatcgacctccgggacagccacccactgcagtatctggagttctctggtcccatccc
gaatcctgaagagctcaatgtccgctttgcagagctggtggatgaattggacctcacagataaaaaccga
gaggctgtgtttgcactaccacccgagaagaaatggcagatctactgcagcaagaggaaggagcaggagg
atcccaacaagctggcaaccagttggcctgagtactacattgatcgtatcaatgccatggccgccatgca
gaatctgtatgaaactgaagatgaggagacagacaagaggaaccaagttgtagaagacctgaagacagcc
ctccggacacaacccatgaggtttgtgacccgcttcatcgatctggaaggcctgacctgtctgttgaatt
tcctccggggcatggaccacaccacctgtgaaagccgcatccacacatctctcattggctgcatcaaggc
tctgatgaacaactctcagggccgggcacacgtcctggcacaacctgaggccatcagcatcatcgctcag
agcctgcgcacagagaacagcaagaccaaagttgcggtgctggagatcctgggtgcagtgtgccttgtac
ccggaggccacaagaaggttctacaggccatgctacactatcaggcgtatgcagccgagcgcactcgttt
ccagactttgctgaatgaactggatcgaagtttaggccgctaccgggatgaggtaaacctgaagacagcc
atcatgtccttcatcaatgctgtcctcaatgctggcgctggagaggataacctggaattccgcctgcatc
tccggtatgagtttctgatgctggggatccagccagtgattgataaactccggcaacacgagaatgccat
cctggacaagcatttagacttctttgagatggtccggaatgaggatgacctggagctagccaggaggttt
gacatggtccacattgacacaaaaagtgcctcccagatgtttgagctgatccacaagaagctgaagcaca
cggaagcctatccctgcctgctctctgtcctgcaccactgcctgcagatgccctacaagcggaatggtgg
ctacttccagcagtggcagctcctggaccgaatccttcaacagattgtcctccaggatgagcgaggggtg
gaccctgacctggccccactggaaaactttaatgtcaagaacatcgttaacatgctcatcaatgagaatg
aggtgaagcagtggagggaccaggcagagaagtttcggaaagaacacatggaactcatgagccggctgga
gagaaaagagcgggaatgtgagacaaagacactggagaaggaggagatgatgcggacgctcaacaagatg
aaggacaaactggcccgcgagtcccaggagctgcgccaagctaggggacaagtggcagagctggtagccc
gacataatgagagctcaaccggcccggtgtcctccccacctccccctggaggtccactcaccttgtcttc
ctccaggacaaccaatgatctgcctccacccccaccacctctcccctttgatagctgcccacccccaccc
gcaccacccctccctcccggagggcctcccatccccccaggtgccccaccctgcttcagctcaggcccac
ccccatctcatgaccccttctccagcaatgaagccccactcaggaaaaagcgcattccccagccttcaca
cccactgaagtccttcaactgggtcaagctgaatgaggagcgtgtttctggcaccgtgtggaatgaaatt
gatgactcgcaggtatttcggattctggacctggaagactttgaaaaaatgttctcggcatatcagaggc
accaggcttgcatgcaggaggggccccagagggagagaggaaatgtgagagacggaggggcggcgagcag
acctctgccagctgtggaggcgagtgcccacagaactgagaaggcttctaggtctatggtatcagctaca
ggtgccaagaaagaactgggctccacggaggacatctacataacctcccgcaaggttaaagagctgtcag
tcatcgatggccggagggcccagaactgtatcattcttctctccaagctgaagctttctaatgatgaaat
acggcaggccatcttgcggatggatgaacaagaagacctcgctaaggacatgctggagcagcttctcaag
ttcatccccgagaagagcgacattgatctcctagaagagcacaaacatgagattgagaggatggcgcgtg
cagatcgcttcctctatgaaatgagcaggatcgaccattaccagcaacggctacaggccctgttcttcaa
gaagaagttccaggaacgcctggctgaggccaagcccaaagtagaagccatcctgctggcctcccgggag
ctgactctcagccagcgcctgaagcaaatgctggaggtggtcctagccatcggcaacttcatgaacaagg
ggcagcgtggcggtgcctatggcttccgggtagccagtctcaacaagatcgctgacaccaagtccagcat
tgacaggaacatctctctgcttcattacctgatcatgatcctggagaagcacttcccagatatcctgaac
atgccctcagagctaaagcacctatcagaagctgccaaagtcaacctggcagagctggagaaggaggtga
gcattctcaggagaggcctccgagcagtggaagtggagttggaatatcagagacaccaggcacgagaccc
caatgacaagttcgtccctgtcatgagtgacttcatcacagtgtctagcttcagcttctcagagctggag
gaccagctgaatgaggccagggacaagtttgccaaggcactgacacatttccgagagcaggatagtaaga
tgcaaccggatgaattctttggcatcttcgacaccttcctgcaggccttcttggaggcccggcaggacct
ggaggccatgcggaggaggaaggaggaagatgagcggagagcacgtatggaattcatgttgaaggaacag
cgtgagaaagagagatggcagaggcagaggaaggtcttggctggcggcgccctggaggagagcggcgagt
ttgatgacctagtgtcagccctgcgctctggagaagttttcgataaggacttaagcaagttcaagcgcaa
ccggaagcggcccgggagccaggttccggaggtcacccgcgagcgggccataaacaggctaaattattga
cttggagactggggactaaccacagaccagcaagcctggagacagggattagaagaaaggagttgaatag
taaaagcaatgacttaagtgattttggaaaaaaaaaaaaaaaaaa (SEQ ID NO: 74)
describes dishevelled associated activator of morphogenesis 2 (Daam2)
mRNA, complete cds. Gene name is Daam2 dishevelled associated
activator of morphogenesis 2. NCBI reference sequence is AY426536.1.
63.
cgtgggtcggtccctccgacacacgagctccaagccgctcctctcttttcctgaagcccttcaaggcggt
cttcggtttccttctttccagccgatcctcttcagccctgtgaagatggtggaccgcgagcaactggtgc
agaaagcccggctggccgagcaggcggagcgctacgacgatatggccgcggccatgaagaacgtgaccga
gctgaacgaaccactgtccaatgaggaacggaacctcctgtcggtggcctacaagaacgtggtgggggct
cgccgctcctcctggagggtcatcagcagcatcgagcagaagacgtctgcggacggcaacgagaagaaga
tcgagatggtccgagcctaccgggagaagatcgagaaggagctggaggccgtgtgccaggacgtgctgag
cctgctggacaactacctgatcaagaactgcagcgagacccagtacgagagcaaggtgttctacctgaag
atgaaaggggactattaccgttacctggcagaagtggccaccggggagaagagggcgaccgtggtggagt
cgtctgagaaggcctacagcgaagcccacgagatcagcaaggagcacatgcagcccacccaccccatccg
gctgggcctggcgctcaactactcggttttctactatgagatccagaacgccccggagcaagcgtgccac
ctggccaagaccgccttcgacgacgccatcgccgagctcgacactctgaacgaggactcctacaaggact
ccactctgatcatgcagctgctccgagacaacctcacgctctggacgagcgaccagcaagacgacgacgg
cggtgaaggcaacaactaaggccccaggtggactggcagcgcccgctgatgctactactgcagtctttat
ttttcccatgagttgggggtcgggtgggggaggggaaagggaggggtgaccttcccagggagaaacccac
gactgtcctgtctttgattgcctctttgacatttttgccaaaataccactagtggaaagtcaggctagct
atgctggtatgggagtagcagcctcacacagacgtccggactgttgtgcaggctcggaacgagcaagcgg
agctgtctgcctttagtttaacttattgctagacggtagggttctaagaagaaaaaccggagatggatgg
ccctcattcagtgcgttctgtggttccagtaaggattcttctgtccatgcccttgtcctcagttccgtgg
ctgttttctctctttggctgtgctttctctctctctctctctctctctctctctctctctctctctctct
ctctctctctcttttttccccctggggtgtacaccagagaagttatagtttaaattacccaactcgacag
gcttagagtggaaggcacaccttttgttagtgagttagacgtggccctgtatttggaagtctctgacggg
ttgacagtattaacactcaattgtagtttacagtatttatacatgacagccatacatgacgccaagccat
gggttctcagttttcctttgtcaggtcggctttcatccgcgtccgggttggtctggctcttcatcatttc
gtaacgtggggagagtcagagctccctgttccgttttggttgatgggtgtttgggagggggctgcgggga
tacttttttttttagttcttctttacccttaaggtcagcccatcacaaaaacagaaaccgttaaataccc
cccacacagagaaaacggagaaaattcaggtctgtatgtcatttatcagagactctgctctcagcagcca
gggctccgtctcagtgtcactgcctgttgaatgttcctcagcacccaagtctactctgcaccttgtgcta
gcagcatgcaggtgccccgaggcgtaggactggagtgtagcgggtgggggattatcatccggtgccagca
gctgagcctacagggaacccactaggaagaggcagttcacttgaggcagcctggtccctaagccagttgt
ccctgcctccctccagccccggcactcctttcccaagtgcttagtggagggtagccgaggttcggcagga
ggccatgctcaccctgtttgacctaagctttcctggtttcccctttttgaatgaaatcgtagttgttgag
attggcttctctctcatgaggtcgctgcacatgtgacattgaaggaagctctgctgctatgtcctacatg
gtccgtctcagccctaggtccctcattctcactgtggatagccctttccataaacacgcccggttgcatg
cagagtgtgcagtttatgtcttctggagaactcatggtctcgtgggttcccaagacgggcagcgccccaa
acttgcttaacttgtcttcagaggcccagcagccactttgtcgcccacttgagctggtgccgtggctgcg
gaaggatgacgcccctctgcacagtcccagcatgccctttctcggagcccctccccatgctagtgtgcca
gcaccccctaaccgagaccgcgcaaggccgcagcagaggaagcttggccaccagagtggacagaggggcc
ggagaggcttcgcccgggggtttgtctccttgtccgcagtgagcgtgtctgtgtcccgcttgtactgttt
ggaaatgacctactggaattgcaaaacctagtttctctttacatttcagccttgacctttgctgtgttct
agaacagtcatgtgagggtagaactgaagagggagagcgggagacctgccgtgagcttggttccttctcc
ttcctttactgttgagaagtgtgcctgcctctgatgctgtccccagtgccgcgcggctgctcctccatcc
tattgatacagtattgtgcatgctggtgttgtatttctatacggtagcttgacatttattaccttacact
gtaggtgttatgtatcctatgacaaaaaaacttaagacttcaatttttttttttaaacttctttttttta
atttaattttttcctttcaggggtaaagtttgctttaccaaatagtgattgtaacagattgatctgtttt
ggatgttgctatagtgacatgcagttctatattttgtttttcaggggggaggggacaaaagaaacaccag
tgttagcttactcttaataatgtctggtgtttgtcatggtgaaactataactatcgcagtgcaggagaac
gcgcggacgttctcagaacgcgcctctgtgctttcttgtcatgttatgcagtgaaaatatttttaaggtc
taatcaagtgattttttcccccccaactccgtgtttcgctaaggaattatttcgcacacggaccattctt
agcagtttcctcagtgatggaatattcatgaatgtgagtcattatgtagccgtcgtacattgagcaaaat
aaacttacagatctgaaaaaaaaaaaaaaaaaaaaaaaaaaaaaaaaaaaaaaaaaaaaaaaaaaaaaaa
aaaaaaaaaa (SEQ ID NO: 75) describes tyrosine 3-monooxygenase/trypto-
phan 5-monooxygenase activation protein, gamma polypeptide (Ywhag),
mRNA. Gene name is Ywhag tyrosine 3-monooxygenase/tryptophan
5-monooxygenase activation protein, gamma polypeptide. NCBI reference
sequence is NM_018871.2.
64.
gcgggaactttccccggggtcggcggaggctcggccgtgccatggaggagggggctagcgccgcgccccc
gctcttgaactgggactacctggagcgctgcttcgcccgccgccgcgtctgcatctccttcggcctgtgg
atctgcgccgcctgctgctggatcgctgcccacacgctgattctctacctgagatgcacaaagaagtgta
gccaagaccagtcgacactgtgcgctgcgttctgcctcctaacgagtctgtgtgacaccgtgggggcgat
tctggccaggcagctcacgatccaggtgttcactggggcctacctagcagctgtcgatttcatgaacttc
atgttcactctctttcctgtctgtggatccaaatccaagtccaagtccggtcagagcagcagggagagga
gaagaaggcatctgagagccagtgtgttcgccctggctctgcctctgagcctgggtccaggctgggcaat
ctggaccgctatccccaaggctccagccccagtccgtgggccacaacggaggctcctgggaagccttctg
caggaaaacccggaagtcttcggctacctgctgggtgctattgctgcctttggctcctgggcatcccgga
tccctccattctccaatatcgtgagactgggttggggctggggctatagggcatctaggggcatcaggag
atcctggaggtgggatgtccctgtgtggggcacacatgtgttttttctgaggagccagtatatgacagca
gtgaggggcgtatgtgcatgggtctgtgtgtgtatgtgtgagtgagtctgtgtgtgtgtgtgtgtgtgtg
tgtgtgtgtgtgtgtgtgtttatgaaggtccatggctaaacagtacagtcctgagacccacaggaacatt
ccaggaaggctgggtgtttctccttacggaaaaaggtcacaggggtggaacaaactagaggctcacaaga
gaacaagaaagacacaggaagtctaacttggttagctacccaaggccttatcggcagatatttccagatc
tctgggctgagggaagacttacgagccaggcctgttgttgccaaatgggacctgtccctgggagctctga
gtcccacggaagacacagatcacggtgtggtgggagggaggtttccttgctctccgaatagaggaagcaa
gtgtttagggtgggaagcaggagagaaggctcctgtgctgcgatggagtgagggcagaaaccattccagt
gtcaggctgcaggtagtctttcacagtggtgcagagggagagtgggaccagaggaggcaacagctaagaa
agggatgtctgtacccaggggaggacgtggagaagccaccatacaggacagtgcctgaatgagctcaggg
attggtgtagacactgcccagccaggttttctccatagaagactctgtggtaatgcccctgcctcggtaa
taggaccagcaccacgcctgatactagaaagtgagctgggcctgttatttaacccgaggagaagcacttt
cccaggagcaccgtgctttagtgccagggtcagaagaacaagggctgggaggagggcagcttgcgactga
gcatgaagagccgggctgtgctttgaggcctgagcctgcaagtcctgtctccaggctaaaggaagagaca
gagatgctgccctggctgcagcagtctctgggtgagcttcacatgtcagggagaccttccatggatccag
ggcagtgtcctgacctcctgttccagttccctcgggcctgcagcgggatggtcaggctggggaactcaca
ggccctcaatagacctgagaggcttccactgtgctgtgggaaccatgcagaggggcaccattacccccag
ccattactggggccagggaccagacccaccctcacatccaggcactccacagttaggtgcttgactgggt
ctctctattcaggggccttcagcaggtggaggccagaacagtggctgggaagaaactagctgagatacag
gggagatgcagcagtccctacagtgccttgtttgtttgttctgtgctgaacattgagccccagacctcac
atgtaccaggcaggcagtcttccacggagctgcatccacagtccatctgagtattccttggggattcaaa
acaaaaatgaaagaaaatcagccaaaatatacttttctcccgaaatttgttccattccttagccaacacc
ctaaatagttctaaatgccgccacttagcaagattttaaaaccagattatacatgttttatacttgcata
tatatatatgtaaatttaaatatatattatgcaatttagtacatagtctttatatgcacatataaatgta
tatataaaatgacttaaatattaatttatatttattaatttattaattgctggagacatggcacatgtat
accatggctcaggttttacgaccacaggacaactttcagacctttctttcttctttgtgggttccaggca
tccatcaaccttaagttgtcaacagcaggtgtctttatctgctgagccatctcactggccctaaagtgac
ctcatgcctacagacaactactaccatgaatattgcatatgtccctttttgtgagccagtccatcctctc
agaatgtctccgtggcttcacaattccatgtcatctctcccttgatctccctgtttggtgtggttccaca
ggggaatagctggccagtggctctttgtaacccattctccctcaccttcccccgctggcatttccaagtt
cctacccactgtcagaagggacggtctcaggtctgctcccaggagtgccccgaatgtgtagagtctcacg
catccctccgtgtcaggcaggtgccaggtgttagggtccagaggagaatcagtattccataggctgcaaa
cagtaagaaaagcccccaccccgttttaagatttaaatcaggttctgaggagtttgccaggcaaaagtag
cagcttgaagaacagctcgatcgatgcctagcagaaggagcgccctttgtaagtggccgctcatagaggg
cttgcccaatgccaccaagagatccagtgtcctcctgcccggcccctcctctctatgcagtgtcggggca
agtcactctcctatatccacctgtggacccggttcctgtcagctctggctggtctcctctacgcctcggc
catcgtggctcatgaccggcagcctgaatacctccttcaggccacaccctggttcctgatttcactgggc
cgagctgcgctggaccttgctatcatctttctgtcctgcgtgataaagagcaggatgagccgagccttca
gatttgctacagctgaagccagagagagtgccgacacacaagcccttttgacctgtgcagagaaggaaga
agaaaaccaggaggccaggacggaggacaagaactcagactgggtgcctctgaccagcctgtcacactgc
aagcccctgaggacaatgacagccatcagtcgctacatggagctgaccattgagcctgcccagcaggcag
gctgtagcgccaccaggctgcccggtgacgggcagacaagcaccagagacgccgcctcgcaagagcctcc
ctcataccctcccatccaggtcatccaggccagagtgtcttccagcagctcctccgaggtctcctccatc
aactcggacctggagcagaagtattgggaagccctaaactcggagcaggcagctggccgtgtcctcactt
gcaataccagatgggcatatttcccgacagtcacctgtgcctgtcttctatcacccaggagtgtttgtga
cacaagaatacatcactcacccac (SEQ ID NO: 76) describes transmembrane
protein 44 (Tmem44), mRNA. Gene name is Tmem44 transmembrane protein
44. NCBI reference sequence is NM_172614.2.
65.
gagcaagaaggtgcaggatgtaggcctgcagaattgtcagacccagaggcggcttttgccactggaagaa
tttccaacataaaacagatgatcagtttgggacaatcgattctgcgaccagagtttataaagctgtccaa
gatctgcccagtcccagatgtcctgggtcctcagggcggctgtggtctgcgcccttctcctgcagctgga
tgccagaccatcctggactcggatccctttgggttcacactcgcttaggtatttctacaccgctgtgtcc
cggcctggccttggggagccctggttcataatcgtcggctatgtggacgacatgcaggtcctgctcttca
gcagcaaggaggagactccgaggatggcaccctggctggagcaggaggaagcaggtgactgggagcagca
gactcgtatagtcacaattcaaggacagctgtctgaaaggaatctgatgaccctggttcatttttacaac
aagagcgtggacgactctcacacactacagtggctgcaaggctgcgatgtggagccagatcggcacctgt
gtctctggtacaaccagctcgcctatgatagcgaggatctccacaccctgaacgaaaacccaagttcctg
tacagtgggaaacagcactgtacctcacatctctcaggacctgaagagccactgctcagatctgctgcag
aaatacctggaaaaagggaaggagaggctgctgcgttcagaacccccaaaggcacatgtgacccatcacc
ccagacctgcaggtgatgtcaccctgaggtgctgggccctgggcttctatcctgctgacctcaccctgac
ctggcagaaggatggggaggagctgacccaggacatggagtttgtagagaccaggcctgcaggggatgga
accttccagaagtgggcagctgtggtggtgcctcttgggaaagagcagagttacacatgccatgtgtacc
atgaggggctgcctgagcccctcaccctgagatgggagcctgcatggtaccaaaagccttggatttggat
tgttgccatggttttcattttgctcatcatttgtctctgtgtggtttgcatatgcatgaagaagaatgca
ggtgggagaggaaggcgtgacacccaagaagcaggcagagacagtccccaagactctagcaagactgttg
tggatgatgaggagatgggggtttgcttttggaagattaagtactgtaagacttgtctaggccactcccc
aggaacttcagttggcgagtctttactgtcaccttgactggatttaggatcatctgggagatgccccttt
gaatggctgggctgtgaggacagcagaccagttcttgccaccctggacagaaacacatctcacctttctg
gctcgaggatctgaacacctgtctcttgcctacttggcttctagtcaggcattttgtcaccttgtcaagg
gtcccagggacacaaaactccctcctctcacccacagcactctgggtcctaccctcagtgcttcagggac
atttaatcacgtcaaattgggatcaatggctttgatgcagaaaagaactgtggactaatagagatagggt
ttaattaaaaaatatatctttttaatttt (SEQ ID NO: 77) describes histocompati-
bility 2, T region locus 9 (H2-T9), mRNA. Gene name is H2-T9 histo-
compatibility 2, T region locus 9. NCBI reference sequence is
NM_010399.2.
66.
tgctttctaggatgggggtgtgagtggaaaccagcgtcgctatggcaaccaggccgggtggtcgggccct
ggagccctgggagccagcgaaggcgagaggcggggcagcgggaggcgccagggctgcgcggcggggcggg
ggcctgggtctcccgggaggagctggggaacaccgggcgcctctcgcggaggtgcacgccaagttctcgg
gtgagtgtgcacgcgtgtgagctggcccaaggctcacgggcgggcgtgagcacgagcacacgtgtcgagt
gtgcactcgcgtgcgttctggggcccgtgtctggccctctgcgccccgcgcccctcccggccgcccgcag
cagctcccgccactcccgccccgcgccgcttctcgctcggtcctcgcccgccgcgccgcagtcgccgccg
ccgcccagcctgcgcccacgggccgcccgtgccgggcacctgttggaggctcagggacaggactgatctg
gcaacccacccagccctttgtgaagccaggcctcctgcctgcccaccagccagcgcagatcatcttttcc
ctcgacacccaggaagggagggcagtgaggttcctacagaccccagccggccactccagctctacacccg
tctcccagtcagaggtgctgcatgaggggccgcaggggcgatcgcatgaccatcaacatccaggagcaca
tggccatcaacgtgtgccctggacccatcaggcccatccgccagatctccgactacttccctcgcagggg
gccaggaccagagggtggcggcggcggcggtggcacgggctgcggggaagccccagctcatctggcccct
ctggctctggccccccctgcggctctccttggggccactacacccgacgatggagctgaggtagacagct
acgactcggatgataccaccgccctgggcacactggaatttgaccttctctatgatcaggcttcctgcat
gctgcactgtagaatcctcagggccaagggcctcaagcccatggatttcaatggcctggctgacccctat
gtaaagcttcacctcctgccaggagcctgcaaggccaataagctaaaaaccaagacacagaggaacacac
tgaaccctgtgtggaatgaggagctgacgtacagcgggatcacggatgatgacatcacccacaaggtgct
caggatctctgtctgtgatgaggacaagctgagccacaatgaattcattggggagatccgagtgcccctc
cgccgcctcaagccttcacagaagaagcattttaacatctgccttgagcgccaggtcccgcttccttcac
cctcttcaatgtctgcggcgctgaggggcatatcctgttacctgaaggagctggagcaggcagagcaggg
acctgggctgctggaagagcgcgggcgcatcctgctgagcctcagctacagctctcggcggcatgggctg
ctggtgggcattgttcgctgtgcgcacctggctgcaatggatgttaacggctactctgacccttatgtga
agacgtacttgagaccagatgtggataagaaatccaagcacaaaacatgtgtaaagaagaagacactaaa
tccggaatttaatgaggaattcttctatgagattgaactctccactctggccactaagaccctggaggtc
acagtctgggactacgacattggcaaatccaatgacttcataggtggtgtgtctctggggccaggagccc
ggggagaggcccagaaacactggaatgactgtctacatcagccggacacagccctggagcgctggcatac
tctgaccagcgagctgccccctgcagcaggggcttaccctttggcttgagtgaacagcagctgtccatca
caggccctctggggccgggtccagtacccaaccttcgcacgagtgtgttgcacgtttacatggggtggga
tgcccctgccccacactacctgtcttatttttgtgagtctctgtgacgggcctgtcttcttgtgagggac
tgtggagagctatattcacatatgcaaacttcctcctgcctgccttgctgttacctgcaaatatgcaacc
acccgtgcacagggccacgtgggagtctcctgccagtgccccaccccatcctgcagctcctttcttggcc
tttccaggctgcctggtcccctgccccacagggagggggtcggattagggaagattagaggaggacgttt
ccaaatagaggaaaggacaaggaaagaaagagccaactctttaatacagagccttcactaaatcccagcc
ctgcgtagctgctcttctaaaggggcggccaccgtaggtctctacctcccgaagatgtagcagacccttt
tggccactcgtttaaataactgttccctggatggggctgggatgtaagggcaggaccctgccaccttcct
cggggacattgtgcatgtttacgccttttctgattgtgtcagtggccgaagcatggcaactgggcatcaa
taaacatctcttgaggaaaaaaaaaaaaaaaaaaaaa (SEQ ID NO: 78) describes double
C2, alpha (Doc2a), mRNA. Gene name is Doc2a double C2, alpha. NCBI
reference sequence is NM_010069.1.
67.
cggacgcgtgggtctcttcggacccttttcgcctgtgggtgacccgacctcaggggcgggtggcaggtga
ggagcgggtcgcagtgaggagggtcgccggtaccctcgaccttcccacagagggcggcgtcctcgcgtaa
tcaaccgctctcggccacgcagagagtagccccacgaggggctggctccttcgggctttagccaattctg
tgtgctccctgtggttttggacaagacaacatggaaacagctaaagatggtgagcagagcccaagtgagg
catctccttcagcccaggcaggtcctgaaaacatcccggagcctatgtctcgggaggaggagagccagcc
attgtaccatgaagagaccattgatcttggtggagatgagtttgcatctgaagagaacgagcccacatcg
gaagggtctcacaacttcggagataagcttaatgagcacatgatggaaagtgtgctcatctccgactctc
ctaacaacagcgagggggacgttggcgacctcggctgtctacaggatgttggggagccccccagaggtgc
cactgatcaccgactgcccagtagcacagacaaagaagtggtggacactctgagtaatggcagtgaaacc
gatggagacgacactcccagagacatctcagacatgacacctgatagcagagcctctctgaaggaagact
caacacaggaagatgtgacgagcatgcctgcactggagaatgctgccacggaggaagtcgggcctaagga
cagcctcgcaccccgagaagagcagacctcagaagtgtcctccaatcagtcctcctccaaagacgagccc
ttgcccgtctgcaccatcttcagccaggctaccgccactccctcccagccacacctgtttctacaggatg
gcttcgagtcccagatggtgaaatcacccagtttcagtagcaccagtgagatgtcagccaagacccctcc
tccgatggtccagcccagtcccagcctcagtacattttttggagatacaatgagctctaattccttggcc
tctgacttctttgattccttcacaacttctactttcatttcagtcagtaatcccaatgcaggttccccag
ttccagaaaagctgtcttcactcactgcccctgtaggagagaagtccccggattccaccagcccatctta
ctccacaaggatggatagatcggaatcaggtgtttctcgggcccctctggatgtccctgaatctccaaag
ccattttcacagatccaggctgtgtttgctggaagtgatgaccccttcgccacagccctgagcatgagcg
agatggaccggaggaatgacgcatggcttcccagtcaggcgaccagagaagtcctgatgtcagtagcaac
ccagcagtatggcactgtgttcatagacaaggagaacctcactatgcctggcctcaagtttgacaacatt
cagggagatgcagttaaagacttgatgcttcgcttcctgggtgagaaggctgcagcaaagagacaggtct
taaatgccagctcagtggagcagtcctttgtggggctgaagcagctcattagctgcaggaactggagggc
agcagtcgacctgtgtggacgcctgctcacagcccacggacaaggctatggcaagaatgggctgcccacc
agccacaccacagactccttgcagctctggtttgtgaggttggcgctgctggtgaagttgggccttttcc
agaatgctgagatggagtttgaaccctttggaaatctggatcaaccagacctatactatgaatactaccc
acacgtgtacccaggacgcaggggctccatggtccccttttcgatgcgtattttgcatgcagaactgcag
cagtatctggggaacccacaggagtccctggaccgattacacagggtcaagaccgtctgcagcaagatct
tggccaacttggagcaaggcttagctgaagatggtggtctgagcagcgtcacccaggaaagcaggcaagc
ctctgttcagctgtggaggtctcgtctgggccgtgtgctgtactccatggcaaactgtctgctcctgatg
aaggactatgtgctggctgtggacgcctacctcacagtaatcaagtactacccggagcaggagccacagc
tgctgagtggaatcggccgcattctcctccagatcggagacataaaaacagctgaaaaatattttcaaga
cgttgagaaagtaactcagaagttggatggactgcagggtaaaataatggtcctaatgaacagagccttt
ctgtaccttggccagaataacttcgcagaagctcacaagttcttcacagagattttgagaatggacccaa
caaatgcagtggccaacaacaatgctgctgtgtgtctgctttacctgggaaagctcaaggactccctgcg
gcagctggaggccatggtgcagcaggaccccaggcactacctgcacgagagcgtgctcttcaacctgacc
accatgtacgagctggagtcctcaaggagcatgcaaaagaagcagagcctacttgaggccgttgctagca
aagaaggggacagcttcaacacacagtgcctgaagctggcttagttccctccaggtctctgtacctgcag
acacttgagcagatgggtcagtaaaagcatttgatgggtgtgcaggagcatgccttcggctggctgggct
accctcagacccgctgacctgcctaccatgtagttgcacaaaactgcctgttggtgaaggtgagccatat
gcatggcattcagaaacagggaacataaggacgtggggttcccttggtttgttcatggaagctaacttgt
tctttggtcacttctgaatactcgtttggtaaataatactgcctgttctcactataggcctaaaaaaaaa
aaaaaaaaaaaaaaaaaaaaaaaaaa (SEQ ID NO. 79) describes tetratricopeptide
repeat domain 15 (Ttc15), mRNA. Gene name is Ttc15 tetratricopeptide
repeat domain 15. NCBI reference sequence is NM_178811.3.
68.
ccggcagtcagaacccgaggtggaaaggcaggctgccgactgcctgtcggaagccgcagggagtgagaaa
gggtgtcgcagagagcgcggcggtggctgagatttcttctgctgaagagaggaaatctcatccaaggcca
caatgtcacttcccaattatccgagtgctgcctgctcaagctaacactgccagagtcctgctccctcggg
aggagtcaagcttacttggcatgaggttcctgctttcctggggttgagagaagcacaactatggcatctc
ttgatgacccaggggaggtgagggaaggcttcctgtgccctttgtgcctgaaggaccttcagtctttcta
tcaacttcagtcacattatgaggaagaacacttggaagatcgtgatgtcaaagggcaaattaaaaatctt
gttcagaaggctaggaaagcaaagaacaagttgttgaaacgagaaggagatgatcgagtggaaccaggga
cccaaggatatgaatcattcagttatggtggggttgatccttacatgtgggaaccccaagagcttggtgc
tatgagaagccatctttctgacttcaaaaaacatcgtgctgcaagaattgaccactatgttgttgaagtc
aataaattaatcatcaggttggaaaagcttactgcatttgacagaacaaatactgagacttcaaagatta
gagcaatagaaaagtccgtggtgccttgggtcaatgaccaggatgtccccttctgtccagactgtgggaa
taaattcagcatccggaaccgccgtcaccactgccgcctctgtgggtctatcatgtgcaagaagtgtatg
gagctcatcggcctgcccttagcacataagctcaccagtgccagcaaggactctctgagcacccacacca
gccccagccagtctcctaacagtgtccatggctcccgccggggcagcatcagcagcatgagcagtgtcag
ctcagtcctagacgagaaggatgatgaccgcatccgctgctgcacacactgcaaggacaagctgctcaag
cgagagcagcagatggacgagaaggagcacacgcctgacatcgtgaagctctacgagaaacttcggcttt
gcatggagaaagttgaccagaaagctcccgaatacatcaggatggcagcatctttaaatgctggggagac
gacctacaatctggagcatgccaatgaccttcgagtggaagtgcagaaagtatatgagctaatagacgct
ttaagtaagaagatcttaaccttgggcttgaaccaagacccttcaccacatccaaacactttgcggctac
agagaatgatcagatactcagctacattatttgtgcaggaaaagttactgggtttgatgtcactgccaac
caaagaacagtttgaagaactgaaaaagaaaaggaagcaggacctggagcagaagaggaccgtggagaga
caggctgctctggagtcacggcggaagcttgaggaaaggcagagtggtttggcatctcatacagccaatg
gggacgtgaggtctcttcgaggcatccctccccccttgcgaaaggctgagggctggctcccactgtcaga
aggtcagggacagagtgaagatcctgaccctctcctccagcagatctataacattacatcgttcatcagg
caggccaaggctgcaggccgcacagatgaggtgcgcacacttcaggagaacctgcggcagctgcaggatg
agtatgaccagcagcagactgagaaggccattgagctgtcccggaagcaggccgaggaggaggagctgca
gcgggagcagctacagatgctccgcaagcgagagcttgaacgagaacaggagcagttcctggcagcatcc
ctgcaaacacggactcgtgttctagaactccgagaagttatacccttccaattggaggccagtcgagggc
ctcacattgatctctcctattccttggatcaggactcctccccagttcagagcagcacagctcctgatat
ccttacacctggctcagctctagcacccatgcacttgtggtctgggcccccagcccttggccaggaaaca
ctccctcagagcaccatgtcacagcaaagtgataaggcctcactcaacccgtttgatgaagacgacctct
ccagccctacagagggtgctatcagccctgctgctgtagaggctttcctgggccctccagctgctgtcac
caaagaatacaatccttttgaggaagatgctgaggaagaggaggtggcagagttgggggcaggaaatccc
ttcaccgacccagacagtccagcacccaacccttttgatgaggatgacggtcccaggcctgccagtccag
ccgcccctggaaacccttttgaagagtgcccctccaccaaccccttcgaggtggacagtgatagtggcat
ggaggccgaggaacacatcgaggaggagctgctcctgcagcaaattgacaacatcaaggcttacatcttt
gatgccaagcagtgtgggcgaatggacgaggttgaggttctaacggagaacctccgggagctgaagtgca
ccctggctaagcagaagggggcccctaattgacagcaatgggcaggaactgggcagagtcttgggcagca
ggatatcaagtgtgaggtggggatgggcaggcatctgaacacacacaggttggggtgggaacagtagtct
cttgctgtgcactttgagacatttccactacagttgagcaagaacgggaaaaacaatcggcattcatgtg
ctggtttttctgtattctttttttaatgaagtattccattttcaggggactttaccattttattttcaag
atacagctctgttgggccaaggcagaaatggctcatgggcacctcttagttcattttctgcggactgttg
ttggtcattatgcagtattatagggcagcccttgacaggcccttcctgtagccaggataggcagggctct
agcagaagtctgtcctgacttggttgttccctttcagcacgtcacttagggagatactgctgtgaagccc
agtgggaccagggcctctttatggatggcaggaatgttgccagtgttgtcccctcccagttaaaggaagt
aaaattcccaagtgtgagggtagtgggctggtggtgtcagtgtgagcttgactgtgagatagtagtgttc
aactttttggcggatgttatggtttggagaagtttctacttatgcagctgtctgatcccctgggaacctc
cttccaggtctttggtagaatgaccgttctgcaggtcacattcaagggcctgttgatttggaattgtgac
tgggaagcttctgtggagtattagcaggtgatggaacacttctgagtcaggatgattctagtaacttcaa
ggccctccagtcagccctcttccttgtggtttatcctgacctgggaaactggtaagatgtactgagaaat
ggaaatatttgaggcacagcctgagaccattccacagccctgaagtacttgaactcttgatagaaaacag
aaagggaagttatgtagcttagtactttggtttacaggaacagatcgattgacagatgaccttgtaaggt
tcttggtgtgctcagctctgggtaatttctttggtgatgatactctttagttaatggacaataactgtcc
actagtcactttcccctcagctttctctgggccatgccttttgttggttactggtgtataagctgaaagt
cttctgtcacattctggtgaagatccatctaggtggtaagaatgttatctgggtgcaaagatctctatgt
tggcagctttaatcccagttcattagtacatgttggggctttggctggatagagagccctgtatggggct
atgggaagggctggagaaggagggagcgtatgaaccctttgagttctgctgacaggtgtttactggcctg
ctagtagattaccagccatttgccagctctgttggtgtcaagatgttctggaaggtaggcagcctggcat
tttatgttacctggggcaggctacctctttctaaagcccaggctccaggcatgcccaccatatctgccct
gcttggtctgctattgtgtcatggacaaagaggcagtgctaacacaagaatgaagctaaattctggtttc
caccattacttttccagactcaaatcaggtgttcagggtggtacaactgtagacaatgcttttcttataa
gactgggcttcttaattagctgtgagtgactcgatgggcagtagtggcacatgacttttatcccagcact
tgggaagtagaggcaggaatttctgagttccaatccagcctggtctacagagtgagttccaggacagcca
gggctacacagagaaaccctgtctcaaaaaacaaacaaataaataaataaacaaaaataaaaattgagaa
aaaaattatctagtgacttattaaatgtacttctttctggaactcaccccaggtagcagtctgactgaga
gcatggagaaaacaaatgcttttagactgactgactattaaaccgggtgaacccagatcccagagggttt
cctgagtttctcattgaattcctcgctgaccatattctgaaagtccaacagaacacgagagacttgtctg
ccccttgtctggatcagctcccagaatgcatgcaggtcagagttggtttgcagagggggtttggcctgtg
agggaaccacagtgccagcttcttaccgtgggtgaatactcgatgattcccaaggatggagcacgtcctt
gatacggtgttggcagatcccttaggtgtaggtcctgttcatagtggacaggcttcttgcttattccctt
ctaacaacactaaggaactggagcctcatcactgcacttaccacatatcatgtctttgtcgtcagacagt
aaaacatctttctaagtcatcatgagccatattgtgtagctatgccatgcggttcaaattgtggaattgg
cctcggtacgaggtcagactagaggagctcggtgtgctggtaatcatgatgactgtcagcaagatggtca
ttgatgcgcatgcactacactggggccttgtattagaggcttttattaaaaacattaaaatatcatttat
gtaaggcttttattaaaaaaataacatgttcatggctggaaaaaaaaaaaaaaaaaaaaaaa (SEQ ID
NO: 80) describes zinc finger, FYVE domain containing 20 (Zfyve20),
mRNA. Gene name is Zfyve20 zinc finger, FYVE domain containing 20.
NCBI reference sequence is NM_030081.2.
69.
tgagagcacgcatgacatgctggggcaagcgtgatttttacagcacgcttcggaagctcgatcaagaccg
ctttgctggatgtttccagaacattcttaaggaatatttgaaaaccaggattggaaaaaaacccaaaaca
acaacaacaacaacagattagatcaactctttgctgtggagattctggcttccaaaaatcctgctgactg
cagcacagctgatgatgtcattgcatcatcacagcggaactggaattcctactgaaattacacttcacga
caaacaagacacaagccaagaacgccactcactgtgttttcccacaaataagtgctataatgtcctgtga
gaattttgtctgtgcttttagagacatgggaatcatttgacagtatttaattttccagttgtgctcctta
tatacaaatg (SEQ ID NO: 81) describes adult male tongue cDNA, RIKEN
full-length enriched library, clone: 2310075C17 product: unclassifi-
able, full insert sequence. Gene name is RIKEN cDNA 2310075C17 gene.
NCBI reference sequence is AK010164.1.
70.
agactgcggcgctgctccattcggccgccgggccccgcggagcccccatcggtgaggcgaggctccagca
ggagacgagcgccccgctacactgcaggggtggcgtagcagcgggcggccgagcgcctgtgagagccaac
tgcgggaggccagcgtgggagcagagatagaaccaggagtcggacccgcgtcccctccgcggaaccccgg
ttcctgtcactgtgggggacatcatggccgtggacgtgacagaatatcatctgagcgtcatcaagagccc
cccaggctgggaggtgggtgtctatgctgccggggccctggctctgctgggaattgctgctgtgagtcta
tggaagctctggacatcggggagcttccccagcccctccccgttccctaattacgactacaggtacctgc
agcagaaatatggagaggcctatgtggaggctaagctgaagcgggtccctccttggaatgaccagcggac
cactactcggggaccacccagccgcaaaggcagccttagtatagaagacacctttgaaagcatcagtgag
ctgggacccctggagctgatgggccgggaactggacttagccccctatggtactctccgaaagtcacagt
cggctgactctctgaattccatctcctctgtgagcaacacctttgggcaggatttcaccctgggccaggt
ggaggtgagcatggactatgatggagcctcccacaccctccacgtggcagtactacaaggcaaagacctc
ctggagcgcgaggaggccaccttcgagtcctgcttcatgcgtgtcagtctgctgccagacgaacagatcg
tgggcatctcccggatccagaggaatgcctattccatcttcttcgatgagaagttctctgtccccctgga
tcccacagccctggaggagaagagccttagattttctgtgtttggtatcgacgaggatgagcggaatgtg
agcaccggcgtggtggagctgaagctgtccgttctggatctcccgctgcagcccttcagtggctggctat
acttgcaggaccagaataaggctgctgacgctgtgggagagatcctgctgtccctcagttacctgcccac
agctgagcggctgactgtggttgtggtgaaagccaagaatctcatctggaccaatgagaagagcacagca
gaccccttcgtcaaagtatacctgctgcaggatgggaggaaaatgagcaagaagaagacggctgtgaaga
gggatgaccccaacccagtgttcaacgaagccatgatcttctcagtgccagccattgtgctacaggacct
gtctctccgggtgacagtggctgagagcagcagcgacggccgtggggacaacgtgggccatgtcatcatt
gggccaggggtcagtggcatgggcaccacacattggaaccagatgctggccacactacgcaggcctgtgt
ccatgtggcatccagtccggcgaaactaacagccagagcaggccaggttgggcatcagagcctcaggagc
ctggcaccatctcagttctatccagcgatgccctctccatagccagctggagctgtgaacaccagggctg
ccccacctcctgggggatttctcatcagccatcttggatcctctgtccagactgtcatggaagaatgggt
gtggctcttcatccggggacccagggtaatttccccactgaggtctacctgtagctggctggggacccac
cagactgacctactgtattgaagagccttaggaagtatgtcccttggcctgcagaaggatgtgggcttca
gaacttgccaactccccttctgccattctcctagaggacagctgttaataaatcttgaggggatagttcc
tgtggtggcaaggctaatattaacaagacatagcactcaggatgccaggaacccaagatgccctctctca
tgctggcaccgagtaacaacccgggcagatgaacttgggaaggagccctgaggggtggggtaacagcgag
gctctctcagacagggaaggagtttcactttgagggtgagagaaaaaatagcatcccatctttaccatgg
gggtcgtggggatcatggtgagcagtcacacaatagaagagacacaaactaccccatcccaaacacagac
tctgcccactacagtggacacaagaagaggccaggagaccaggcccaacagaagccagtaggccgtatga
aggctcccctgaccatccactctgagggaaccagaagtccccagtggcccaggtccagatccttgtgtgg
atccaaagcaaatcagcacagattccacagacaccagtgttggctcgcgtccctccaggccaggcttctc
tagaaaggaaggtgtcaagccggtctaatccaaccctaggaattgtaggtcaagttccctaaaactaaga
ctgctccttagtcttgagccagtaggaactgggggaaatggtgttcctaagcaaagggcttggttgtaca
ccactaccagaatcgttctttggtcagagttattgaggggatcgcatcatacgtgaggtgctggctgtca
cttgagaaaggcagttctcctagagaaatggtggcttttggctccttctctctggaacctgaagattttt
ttgttttttgatttttgggttttttttttttttttccgagacagggtttctctgtgtagccctggctgtc
ctggaactcactttgtagaccaggctggcctcaaactcagaaatctgcctgcctctgcctcccaagtgct
ggaattaaaggtgtgtaccaccacggcagactcgaacctgaagatttaaaagggcagtgggcgtgggagt
ggtcagaagagggctttggtccttggccctagtgaatcatggaggcccagatggaagcaggtctaataat
ggtcaatgataaggagaagggcagatagaatagggacacaggtcaacaggacaccagccacagctggacc
cactgaagcttctcaccctgtggccaatattcaagaggtgaaaacgggccagggatagtggtggctgggc
caggccttgtcatctggaagcccccagactctttgtaatccaatggctttgcagggaagaccacaccccc
caccctccatccccttattccttctttccttccttctatactgcatgaaacagtgtgcccagatggctgt
ggtggcagccagggatgattcttgaaacaaaataaaactggggcaggaaggaggc (SEQ ID NO:
82) describes synaptotagmin XII (Syt12), mRNA. Gene name is Syt12
synaptotagmin XII. NCBI reference sequence is NM_134164.2.
71.
aagcactcatagtcagctggggatacaacccctaaaaacagcacacccaccatgtttaacctcatgaagaaagat
aaggacaaagatggcgggcggaaggagaagaaggaaaaaaaggagaagaaggagaggatgtctgcggcagaactg
cggagcctggaagagatgagcatgcgccgtggctttttcaacttgaaccggtcctccaagcgggaatctaagacg
cgcctggaaatctccaaccccatccctatcaaggtggccagtggctctgacctgcacctgacggacattgactct
gacagcaatcggggtagcatcatcctggactcaggacacctaagcacagccagctccagtgatgacctcaagggt
gaagaaggcagcttccggggctctgtgctgcagcgggcagccaagttcggttcactggccaagcagaactctcag
atgattgtcaaacgtttttccttctctcaacggagccgggatgagagtgcctcggagacctcaaccccctcagag
cactctgcagccccatcacctcaggtagaggtgcggacgcttgagggacagctaatgcagcatcctgggctaggc
attcctcgaccaggaccacggtctcgagtccctgagctggtgactaaaagattcccagctgatctacgtcttccc
gctctggtgccaccaccacctcctgctctccgggagctggagttacaacgaagacccacaggagactttggcttc
tcactgcggcgcacaaccatgctggatcgggcccctgagggtcaggcttatcgacgggtggttcactttgctgag
ccaggggcaggcaccaaggacctggccttggggctggtaccaggagatcgactggtggagattaatggacagaat
gtggagaata
aatccagggatgaaattgtggagatgatccggcagtctggggacagtgtgcggctcaaggtacagcccatcccag
agcttagcgagctgagccggagctggcttcggactggcgagggacaccgcagggagccggctgatgccaagacgg
aagagcagattgccgctgaagaggcctggtatgagacggagaaggtgtggctggtccatagagatggattctcct
tagccagccagctcaagtctgaggagctcagcctacccgaggggaaggcgcgtgtgaagctggaccatgacggag
ccatcctggatgtggatgaagatgatatagagaaggctaatgctccttcatgtgaccgtctagaagatctggcct
cactggtatacctcaacgagtctagcgtcctgcatacactgcgccagcgctatggggctagcctgctacacacct
atgctggccccagcctgcttgtccttagcacccgaggggctcctgctgtgtattcagagaaggtgatgcacatgt
tcaaggggtgtcggcgggaggacatggcaccccacatctacgctgtagcccagactgcatatagggcgatgttga
tgagtcgtcaggaccagtccattgtccttctgggtagtagtggcagtggcaagactaccagctttcagcatctgg
tgcagtatctggctaccatcgcgggtaccagtgggaccaaggtgttctcagtggagaagtggcaggctctgtcca
ccctcctggaagcctttgggaacagccccaccatcatgaatggcagtgccacccgcttctcccagatcctctccc
tggactttgaccaagctggccaggtggcttcggcctccatccagacaatgctcctggagaagctgcgtgtggccc
ggcgcccagc
cagtgaggctactttcaatgtcttctactacttgctggcctgtggggatgccaccctcaggacagagctccacct
gaaccacttggcagagaacaatgtgtttggaattgtgccactgtccaagcctgaggagaagcagaaggctgcaca
gcagttcagtaagctccaggctgccatgaaggtgctggccatctcccccgaggaacagaagacctgctggctcat
cctggcttccatctaccacctgggggctgctggagctaccaaagaagctgctgaagctggacgaaagcagtttgc
ccgccacgaatgggcccagaaggcagcatacctgctgggctgcagcttggaagagctgtcctcggctatcttcaa
acaccagctaaaaggaggcaccctgcaacggtccacctccttccgtcagggccctgaggagagcggcctgggaga
gggcaccaaactgagtgctctggagtgcttagagggtatggcgtctggcctctacagtgagctctttactctcct
catctccttggtaaacagggctctcaagtccagccagcactcactctgttccatgatgatcgtggacacaccagg
cttccagaaccctgaatggggcggctcggcccgtggagcctccttcgaggagctgtgccacaactatgcccagga
caggctgcagaggctgttccacgagcgcaccttcctccaggagttggagagatacaaggaggataacatagaact
ggcgtttgatgacttggagccagttgcagatgactccgtggctgctgtggaccaggcttcccacctggttcgctc
actggcccatgccgatgaggcaagagggctactgtggctcctggaagaggaggcactggtgcctggtgcgacaga
ggatgccctc
cttgatcgtcttttctcctattatggcccccaggagggtgacaaaaaaggccagagtcctcttctgcgaagtagc
aaacctcggcactttctcctgggccacagccacggtaccaactgggtggagtacaacgtggctggctggctgaat
tacaccaagcagaacccagccacccagaatgcaccccggcttctgcaggactcccagaaaaagatcatcagtaac
ctgtttctgggccgggcgggcagtgccacggtgctctcaggctcgattgcaggcctggaaggtggctcccagctg
gccttgcgccgggcaaccagcatgaggaaaaccttcaccaccggcatggccgctgtcaagaagaagtcgctgtgc
atacagattaagctgcaagtagatgccctcattgataccatcaagaggtccaagatgcatttcgtgcactgcttc
cttcctgtggccgagggctggcctggggagccccggtctgcctcttcccgcagagtcagcagcagcagtgagctg
gacctgcccccgggagacccctgtgaggctgggctgctacaactggatgtgtccctgctccgagcccagctccgg
ggatcccgcctgcttgatgcgatgcgcatgtaccgccaaggttacccagaccacatggtgttttctgagttccgc
cggcgctttgatgtcctggctccacacctgaccaagaaacatggtcgtaactacatcgtggtggatgagaagcgg
gctgtggaagagctgctggagtccctggacctggaaaagagcagctgctgtctgggcctaagccgggtgttcttc
cgggcaggcacgttggcacggttggaagaacaacgagacgagcaaaccagcaggcacctgaccctgttccaggca
gcctgccggg
gctaccttgcccgccagcacttcaagaagagaaagatccaggacctggccattcgctgtgtgcagaagaacatca
agaagaacaagggggtgaaggactggccctggtggaagctcttcaccaccgtgcggcccctcatccaagttcagc
tgtcagaggagcagatccgaaacaaagacgaggagatccagcagctgcgcagcaagcttgagaaggtggagaagg
agaggaacgagctccgactcagcagtgaccggctagagacccggatctcagaactgacatcggagctgactgatg
aacgcaacacgggagagtccgcctcccagctgctggacgcggagacagccgagaggctccggaccgagaaggaga
tgaaagagctacagacccagtatgacgcactgaagaagcagatggaggtgatggagatggaagtgatggaagccc
ggctcattcgggcagccgagatcaacggggaggtggatgatgatgatgcagggggagagtggaggctgaagtacg
agcgagctgttcgggaggtggacttcaccaagaagcggctccagcaggagctggaagacaagatggaggtggaac
agcagagcaggaggcagctggagaggcggcttggggacctgcaagcagatagtgatgagagtcaacgggcactgc
agcagctcaagaagaagtgccaaaggctcacagctgagctgcaggacaccaagctgcacctggagggccagcagg
tccgaaaccacgagctggagaagaagcagaggaggttcgacagtgagctttcccaggcccacgaggagacgcagc
gggagaagctgcagagggagaaactccagcgggagaaggacatgctcctggctgaggctttcagcttgaagcagc
aaatggagga
aaaagacttggacattgcggggtttacccagaaggtggtttccttggaggctgagcttcaggacatttcttctca
agagtctaaggatgaggcttctctggccaaggtcaagaagcagctccgggacttggaggccaaggtcaaggacca
ggaagaggagctggatgagcaggctgggagtatacagatgctggagcaggccaagctgcgtctggagatggagat
ggagagaatgagacagacccattccaaggagatggagagtcgggatgaggaggtggaagaggcccggcagtcatg
tcagaagaagttaaaacagatggaagtgcagctcgaggaggagtatgaagacaagcagaaggcgctgcgggagaa
acgggagctggagagcaagctctccacgctcagtgaccaggtgaaccagcgggactttgaatcagagaagcggct
acggaaagacctgaagcgcaccaaagcgctgctggcagatgcccagatcatgctggaccacttaaagaacaatgc
cccgagcaagagggagattgcccagctgaagaaccagctggaagagtcggaattcacctgtgcagcagctgtaaa
ggcacggaaagcgatggaggtggagatggaagacctgcacctgcagattgatgacatcgccaaagccaagacagc
gctggaggagcaactgagtcgacttcagcgtgagaaaaacgagattcaaaaccggctggaagaggatcaggagga
catgaatgagctgatgaagaagcacaaggcagctgtggcccaggcctcccgggacatggcacagatgaacgatct
ccaggctcagattgaagagtccaacaaggagaagcaagagctacaggagaagctacaagctctgcagagccaggt
ggagttcctg
gagcagtccatggtggacaagtccctcgtcagcaggcaggaagcgaagatcagggagctggagacacgcctggag
ttcgaaaagacccaagtgaagcgcctggagaacctggccagtcggctcaaagaaaccatggagaagctgactgag
gaacgggaccagcgcgccgcagctgagaaccgtgagaaggagcagaacaagaggctccagcgacagcttcgtgac
accaaggaggagatgagcgagcttgccaggaaggaagccgaggctagccgcaagaagcatgaactggagatggac
ctggagagcctggaagctgctaaccaaagcctgcaagccgacctaaagctggcgttcaaacgcattggggacttg
caagctgccattgaggatgagatggaaagtgacgagaacgaggacctcatcaacagtgagggggactcagatgtg
gactcagagctggaggaccgggtcgacggggtcaagtcctggttgtcaaagaacaagggaccttctaaggcacct
tctgacgatggcagcttgaagagttccagcccaaccagccactggaagccactcgcccctgacccatcggatgat
gagcatgatcctgtggacagcatctccagaccccggttctcccacagctatctgagtgacagcgacacagaggcc
aagctgacagagaccagtgcatagcctgggatggctcacgactcttccaccccacagcctctcccaggatagagg
ggcaccactgcccccacctgactgccgatctgcatggaaaacaccttggctttctgtcagggggactttccaggc
tgtgggccgtctgacagctccacgcggcagaggtgggcgaagcagagtctctccaaagagagcttccgctcttgc
ctctgaacgc
catgctcttagctcccgctctgggccactatggaccacgtcaggtggcctggcattccatggagccggtggtgga
cctccatctcagcctcctgctcagggatggtggataggttacctgctgggccgctcgaagtcgctgggccccatg
ggaaagagtcaggagggagcagtgatgcctcccctctgctggggtgagggctttctcttctcagtgcctgctgtc
tttttactgtttaatttaaatacccaaacctccatgcagctgcatccctgagagtgggaggggctgccgctgcag
ctggggcctgtgggatgccttgggaatctcccctccatcttcacttctccagaggcccttttctcctgcagcctg
ctgcaggggacagcggttagcagaccggggccctggttctttgaaggcagggtggggaggatggcagctgggtgc
ttctggcttggaccgagccagcctcgttagagtattaagtgcaatgagcagttatttaagagctgatctcttcca
cctgcacccacccgcggcttccagctctgcctgcctctgaccgtattatagccggggccgctcattcggcgctga
ggacctgtggtgggcacagtggaaggtagatgttttggtctgtttatcagactggagtcctaaccctttgtcatc
tttcttctgcccataaccctgtccccacacacttaccccagacacagcaacaagtggtctgttacttgttacttc
tcggtttcttaccataaagctcatctgacttttccactggatctgggggccccaaggcagtacctggtgctggac
ctgtttgccatcccccttcccttctgccattgctttgtactcattgggctgtgctctccctgtggacctgattga
tgtaaggaaa
taaaaggccccttccctcctggcaaaaaaaaaaaaaaa (SEQ ID NO: 83) describes myosin
XVIIIa (Myo18a), mRNA. Gene name is Myo18a myosin XVIIIA. NCBI reference
sequence is NM_011586.1.
72.
agtccctggaagcagacgtttcggccacagacccagagaggaggagctgacaatcaggaggcgtgagccgcctgg
agtctgcagaattcgtggtgtgaatgaactgggggcatcttgggcacagggattgccccccctccttccccgcct
cgggccacagttgagtagtggggcattttttttcaccttcttgtgaagaattttttttattatttgttgtaaagt
cttttgcacaatcacgcccacatttggggttggaaagccctaattaccgccgtcgctgatggacgttagagaggg
agcgcctcgccgcggaacagtcgcctgcgcgccctcgtcggacccgcggctcctgcactgtgtccccgctcggcc
ctgcgcttgctgctcgcccgcgcgcgccggcgccctctcggttcctgggcacatttccacgctataccaactcct
ctgcccgagtccgggcgccagtgctcgcttccgctccgggtcgctgcgcccacccgacgcgcccaggaggactcc
gcagccctgctttggattgtcccccaaggcttaaccccgacgcttcgcttgaattcctcggccgccttcgctcgg
gtggcgacttcctctccgtgccccctccccctcgccatgaagaagcccattggaatattaagcccgggagtggct
ttggggaccgctggaggtgccatgtcttccaagttcttcctaatggctttggccacgtttttctccttcgcccag
gttgttatagaagctaattcttggtggtctctaggtatgaataaccctgttcagatgtcagaagtatatatcata
ggtgcacagcctctctgcagccaactggcaggactttctcaaggacagaagaaactctgccacttgtatcaggac
cacatgcagt
acattggagaaggtgcgaagacaggcatcaaggaatgccagtaccagttccggcatcggagatggaactgcagca
cagtggacaatacttctgtctttggcagggtgatgcaaataggcagccgagagacggccttcacgtacgcggtga
gcgcagctggggtggtgaacgccatgagccgagcatgccgggagggcgagctgtctacctgtggctgcagccgcg
ctgcgcgccccaaggacctgcctcgggactggttgtggggcggctgcggagacaacatcgactatggctaccgct
tcgccaaggagttcgtggacgctagagaaagggaacgaatccacgctaagggttcctatgagagcgcacgcatcc
tcatgaacttacacaacaatgaagcaggccgtaggacagtatacaacctggcagatgtagcctgtaagtgtcatg
gagtgtctggctcctgtagcctcaagacgtgctggctgcagctggcggacttccggaaggtgggcgatgccctca
aggagaagtatgatagcgcggcggccatgaggctcaacagccggggcaagctggtgcaggtcaacagccgcttca
actccccgaccacgcaggacctggtctacatcgaccccagtccggactactgtgtgcgcaacgagagcactggct
cgctgggcacgcagggacgcctgtgcaacaagacctcagaggggatggacggctgcgagctcatgtgctgtgggc
gtggctatgaccagtttaagacagtgcagaccgaacgctgtcattgcaagtttcactggtgctgctatgtcaaat
gcaagaagtgcacggagattgtggatcagttcgtgtgcaaatagtggtgtgcctgcccttcacccagtcccactc
ccaggaccca
cttatttatagaaagtacagtgcttctggttctttttatttctcccccaagaattgcagctggaaccatgtgttt
tgttttgttttattttgttttttcttttctgttaccatctaagaactctgtggtttattattaatattataatta
atatttggcaatagtgggggaaactaagaaaaatatttattttgaggatctttgcaaagttagtacaaaatttct
ttcttctgatgctacaggataaaggggaaaaactatgtattcgaacttagctgtgcagttgggggttcacatcta
gaaggtgtaggagccattttcttctcaaacagagagtcctttgagatgggtggtatccaggtgaaggaggaggta
cagacccatgaataacagttcctgtgaccaaaatgaattgcaggtgctctggtacaaaagatcttaaatatagat
atattaaatatacatatatgccaaaaatacagaatatgagacactccctaacccagaggttaccagcctggtttt
gtgggttttttgttttgttttgttttttctttttttgggttttgtttgtttgtttgtttgtttgtatttttggtg
tgtgtgtgtgtgtatttctagaatgatcttttagaaggtacaagcaagaatctcatatcttcagaagcaggcata
tcatgtatgttactgtgtcccacctacagatactccattcatgaatgggcctttttctaacagttcatgaatatt
ggggagccggtgggctgggggagggaggtccccagaaattagaaaacttgaagtttcctacattgaggccataat
cttgtgttagcccagctgattcttaataccagacttttagatccataaaggaatttttgactaaaaaaaaaaaat
cttgttttga
aagccatcttattttcttaaaaatgaaaaattacccatgaatcccatttgcaacccctcacccccacaggcaaca
agaaagtcccatgtagttgagcactgcgaacacctctgtgaggagatgatggcagccatcttcctgcatgatccc
atgccctttctggactctctgctggccatgcttccgaatggcagccctggtggacactcactgctggtagggcag
aaaatgtacacgaggagccatgttcagaaccagccacttaggggttgttctctgaggcttttctttggaggtacg
gtaacttgatgtgttttgatgatatctcttggcccagggagtccacagaggtgttgcagctgtttggttgttatc
ctcctgcgtttagactttccatttgtgcttttcctattaccctgcaggtgtaccctaaaactgttcctagtgtac
ttgaacagttgcatttataaggggggatgtggtttaatggtgcctgatatctcagtttttttgtatataacatat
atataaatatacatatataaatatagatataattatatctcagtgcagtctgggatttagacctacagttttctc
tgggcttgctctctgcctggagtatcgtccttcattgcagtccaattgggatttctttttttccaaaaattttga
gtcttaacattgacctgtgacaggatcctaccacgaataccaggaagcaagctaagactcggaggaagctctcag
ggctcatgtcctgaatgtatgttggttagaaagtagcctttctgcttcctgcccatggccagttctccaccctct
ctttggtgttctttgtggggagggcactgtggtttgtcgcagccctggacttcgagaggctcccagaacccagga
tcaccagcct
cctgtctgtttgcttcactcctttcccagggaggacttgggactgtcctgtctgacaggacggatctgagttccc
gaagcaaaccagctcaccacatagatagctagtttaaacaatgttttaaaataagggcacctctgtttcaaaagt
gacatctgctgtgttgttttcgaggcctgatactcttacaaggtttgaaaaaaaatgtgtgtatccattcatggg
cttggtagccttctggtcacctcagtcctgtggctcttaacttattgcccaacaatattcatttcccctcagcta
caatgaattgcaagcaaaagatgttgaaaaaaagcactaatttagtttaaaatgtcactttttggtttttattct
acaaaaaccatgaagttctctctctctctctctctctctcttatttgttaaatcagattatgttctttttttgtt
tttgtttttagtgattcatgtttatgagcagagtggagtttaacaatcctagctttaaaaaaaacctatttaatg
taagatattctacgcatccttcagatattttgtatatcccctatggcctttattctgtacttttaatgtacatat
ttctgtcttgtgtgatttgtatatttcact
ggt (SEQ ID NO: 84) describes wingless-related MMTV integration site 5A
(Wnt5a), mRNA. Gene name is Wnt5a wingless-related MMTV integration site
5A. NCBI reference sequence is NM_009524.2.
73.
gatgatgctctgcgttgctgtcatggctgcctccatgcatgtataatctcacatgggagtctaagtttgtgagcg
ctatccgtcgtaacaagacgccggcgactttggaccctcctcctctcgtttttctcctccttagtgggacactgt
atcgggtgcagcgatgccgggaatgatgtggacgccggagctggcgcttctgaggggcttctccacggaggcgga
gcggctggtttggaagcaggagggcatctgtggttcagatattggagttttcttagaattgctgttagaagggag
ctatgaggctttgttcttccattcaacgacacaaactattttaaattcaacaatgatggctgaagaaaagattga
cagctacctggagaagcagatagtgaatttcctggattgctctacagatttggaagaaattgaaagacagcagct
ggtattcctacttggtgtgagcagcttgcagctttttgtccagagcaactggactggacctcttgtggacttgca
tcctcaggatttcttgccatctggtctgctggagcaattcagtgaggtcaaaggactggatgctattattatggg
tctcctcattctagatggcgaatcagtctacagcctcacttcaaagcccatactgctgctaatagcacgcattat
cctagtgaacatcagacataaattgacagctctccagagcttgccatggtggacgttgagatatgtgaatattca
tcagcaattgcttgaggagcgatcacctcagctttttgctcttgccaaaaactgtattgatcaagtgatgaaaca
ggagaatttatttgaaggtgattcaggtcgacttttggctattcaatttcatctggaatgtgcacatgtattttt
atattattat
gaatataaagaagcaaaggaccagttcagtactgctaaagacatcagcaagttagagattgacctgacaggtgcc
ttgggaaaaaggacacgattccaggaaaattacgtggcgcagctgattgtagatgtcaggagaaaagaggctgtc
cctttcagttgtgagttcagcccagcacccacgccccaggagtgtttggccaagaatcttgagctcaatgatgat
actgttctgaatgaaataaagttagcagatagtgaacggtttcagatgccagacctgtgtgcggaggagcttgcc
gtggtccttggagtctgcacaaatttccagaagaacaacccagttcataagctaacggaggaggagctgctggcc
ttcacatcgtgtttactttcacaaccaaagttctgggccattcagatgtcagccttgatcctccggacaaaactt
gagagaggaagcacacggcgagtggagcgggccatgaggcagacccaggctcttgcagaccaattcgaagataaa
gccacatctgtactggaacgtctgaagattttctattgttgtcaagtaccccctcactgggctgttcagcgccag
ctcgcaggtttgctgtttgagttgggatgcacctcctcagccctgcagatatttgagaagctggagatgtgggag
gatgtggtcatctgccacgagagggctgggcggcacgggaaggcagaagaaatacttaggcaagaactggagaaa
aaggaaacaccaggtttatattgcttacttggagatgtcctgcaggaccattcctgctacgacaaggcctgggag
ctgtcccggcatcgcagtgcacgtgcccagcgctccaaagctctcctccatcttcggaacaaggagtttcgggaa
tgtgtggagt
gcttcgagcgctcagtgaagatcaatcccatgcagctcggggtctggttctctctaggctgcgcctacttggcct
tggaagactatggtggatcagcaaaggcgtttcagcgttgtgtgaccctagaacccgataatgctgaggcttgga
acaatttatctacttcctatattcgattaaagcaaaaagtcaaagcttttagaacgttacaagaagccctcaagt
gtaactacgaacactggcaaatctgggaaaactacattctcaccagcaccgatgtcggggagtttggggaagcca
tcaaagcttaccaccggctcctggacctccgagacaaatacaaagatattcaggtccttaaaattctggtccagg
cagtggttaatgatatgaccgaccgaagtggagctgttgcctccagcctcaaaggcaagctgcaggagttgtttg
gcagaataacttcaagggtgaccaatgacggagaagtctggagactgtatgcccaagtgcatgggaatgggcaga
gtgagaagccagatgagaacgacaaggcattccagtgtctctcgaaggcgtacaagtgtgacacacagtccagtt
gttgggagaaagatgctacagcatttaaggaagttgttcaaagagccataggactcgctcatgtggctatgaaat
gcgctgagagcaagtccattccccaggaagctgtacaaacactttcttcagttcgactcaatttacgtggcttgt
tatccaaagcaaagcagaattttacagatgtggtaagtggagaagtctctggagagctcgacagtgaaatagcag
ctgtgaatgctctggaggcagagctgcaagacctcagcaaccagcttcgaaaccggtactgactgaaaagcagct
tgtggcgcag
tctcttctgctttctccaattcctccagtacctgtatttctgaaacacaagatacccgtttttaaaataaatttg
tttgctggcaaacgcc (SEQ ID NO: 85) describes tetratricopeptide repeat domain
27 (Ttc27), mRNA. Gene name is Ttc27 tetratricopeptide repeat domain 27.
NCBI reference sequence is NM_152817.2.
74.
gacctggagagccaggaagatggtggtgatggcgcgagttccgcgttccgagcggccggaccttgtttttgagga
agaggaccttccctatgaggaagaaatcatgaggaaccagttctccgtcaaatgctggctccgctacatcgagtt
caaacagggggcccccaagccccgactcaaccagttgtatgagcgggcactcaaactgcttccctgcagctacaa
actctggtaccgatatctgaaggcacgccgggcacaggtgaaacatcgctgtgtgactgatcctgcctatgaaga
tgtcaacaactgccatgagagggcctttgtgttcatgcacaagatgccacgcctgtggttagactactgccagtt
tctcatggaccagggaagggtaacacatacccgccgcacctttgaccgtgccctcagggcactgcccatcacaca
acactctcgcatctggcccctgtatctacgtttcctgcgctcccacccactgcctgagactgctgtgcgaggcta
ccgtcgcttcctcaagctgagtcctgagagcgctgaggaatacatcgagtatctcaaatccagtgaccggctaga
cgaggctgcacagcgcctggccaccgtggtgaatgatgagcgctttgtatccaaggctggtaaatccaactacca
gctgtggcacgagctgtgtgaccttatctcccagaatccggacaaggtacagtctctcaatgtggatgccataat
ccgtggtgggctcacccgtttcactgaccagctgggcaagctgtggtgctctttggcagactactatatccgcag
tggccacttcgaaaaggctcgggatgtgtatgaggaggccatccgcacggtgatgaccgtgcgggacttcaccca
ggtgttcgac
agctacgcccagtttgaggagagcatgattgcagcgaagatggagactgcctctgaactggggcgtgaggaggag
gacgatgtggacctagagctgcgcctggcccgcttcgaacagctcatcagccgacggcccctgcttctcaatagt
gtccttctgcgccagaacccacaccacgtccacgagtggcacaagcgtgtagccctccatcagggccgccctcgg
gagattatcaacacatacacagaggctgtgcaaacagtagaccccttcaaagccacaggcaagccccacacactg
tgggttgcattcgccaagttttacgaagacaatggacagctggatgatgctcgtgtcatcctagagaaggccacc
aaggtgaacttcaagcaggtggatgacctggcaagtgtgtggtgccagtgtggggagctagagctcaggcacgag
aattacgatgaggccttaaagctgctgcggaaagccacggctttgcctgcccgccgagctgagtactttgatggt
tcagaacctgtgcagaaccgtgtatataagtcgctgaaggtgtggtccatgcttgccgacttggaggaaagcctg
ggcactttccagtcaaccaaggccgtgtatgaccgaatcctggacctgcgcatagccacgccacagatcgttatt
aactatgccatgttcctggaggagcacaagtactttgaggagagcttcaaggcatatgagcgtggcatctcactg
ttcaagtggcccaatgtttcagacatctggagcacctacctgaccaaattcatctcgcgctatgggggccgaaag
ctggagcgtgcacgggatctctttgagcaggcactcgatggctgcccaccaaaatatgccaagactttatacctg
ctctacgcac
agctggaggaggagtggggccttgcccgccatgccatggctgtgtatgaccgtgcgaccagggcagtggagccag
cacagcagtacgacatgtttaacatctacatcaagcgagctgctgagatctatggcgtcacccacactcgtggca
tctaccagaaggccattgaggtgctgtctgatgagcatgcccgtgagatgtgcctgcggtttgcagacatggagt
gcaagctcggtgaaatcgaccgcgcccgggccatctacagcttctgctcccagatctgtgatccccggacaactg
gggcattctggcaaacatggaaggactttgaggttcggcacggcaatgaggataccatcagggagatgctgagga
tacggcggagtgtgcaggccacatacaatactcaggtcaacttcatggcttcacagatgctcaaggtgtcgggca
gtgccacaggcaccgtgtctgacctggctcctgggcagagtggcatggatgacatgaagctgctggaacagagag
cagaacagctggcagctgaggcggagcgggaccagccccccagggcccagagcaagatcttctttgtgaggagtg
atgcatccagggaggaattggcagagctggctcagcaggccaaccctgaggagatccagctgggcgaggatgagg
acgaggacgagatggacctggagcccaatgaagtccgactggaacagcagagtgtaccagctgctgtgttcggga
gcctaaaggaagactgactccctccacactttttcttccatggtcactcccccataactacccttctaatacatc
ccctctttgt (SEQ ID NO: 86) describes XPA binding protein 2 (Xab2), mRNA.
Gene name is Xab2 XPA binding protein 2. NCBI reference sequence is
MM_026156.1.
75.
cgggcgccgagcgccgagcgccgagcgccgcccgccgagcgccgcgtcttcccgcgccgccgccccgagctccgc
ccgccgcgcgctccgcccgccgcgccgccaggctgcccggcggccgcccagagcgaggatgcggcggcgctcgcg
gatgctgctgtgcttcgcgctgctctgggtcctgggcatcgcctactacatgtactcggggggcggctccgcgct
ggccgcgggcgggggcggcgccggcaggaagggtgactggaatgacatcgactccattaagaagaaagaccttca
ccacagcagaggagatgagaaagcacagggtgtggagaccctccctccaggcaaagtccggtggccagactttaa
ccaggaggcatacgttggcggtacaatggttcgctctgggcaggacccgtacgcacgcaacaagttcaaccaggt
ggagagtqacaagctgcacatggacagaggcatccccgacacccggcatgaccagtgtcagcgcaagcagtggcg
agtggacctgccagccaccagcgtggtgatcacattccacaatgaagccaggtcggccctgctgcggacggtggt
cagtgtcctcaagaggagtccacctcacctcatcaaggagataatcctggtggatgactacagcaacgaccctga
ggacggggccctcttggggaaaatcgagaaggtgcgggtcctcagaaatgaccggagagaaggtctaatgcgctc
tcgggtccggggtgccgacgccgcccaggccaaggtcctgaccttcctagacagtcactgcgagtgtaacgagcg
atggctggagccactcttggagcgggttgctgaggacaggacccgagttgtatcccccattatcgatgtcattaa
catggataat
ttccagtatgtgggcgcctctgctgacctcaagggtggttttgactggaacttggtgttcaagtgggattacatg
acaccagagcagaggcgatcccggcaggggaacccagttgcccctataaaaactccgatgatcgctggcggcctc
ttcgtgatggacaagctctattttgaagagctggggaagtacgacatgatgatggacgtgtggggaggagagaac
ttggagatctcattccgagtgtggcagtgtggtggcagcctggagatcatcccgtgcagccgcgtgggccacgtg
ttccggaagcagcatccctacacgttccccggtggcagcggcactgtctttgcccggaacacacgccgggcagct
gaagtctggatggatgagtacaaacatttctactacgcagcggtgccttctgcacgaaacgtgccctatgggaat
attcagagcaggctggagctcaggaaaaagctgggctgcaagcccttcaagtggtacctggacaacgtctacccg
gagctgagggttccagaccaccaggacatagctttcggggccttacagcagggaaccaactgcctggacaccttg
ggacacttcgcggatggagttgttggaatttatgagtgtcacaacgctggaggaaaccaggaatgggccttgaca
aaggagaagtcggtgaagcacatggacctgtgccttactgtggtggaccgttcgcctgggtcactcatcaggctg
cagggctgccgggagaacgacagcagacagaagtgggagcagatcgaaggcaactccaaactgcgacacgtgggc
agcaacctgtgcctggatagccgcactgccaagagcggaggcctgagtgtggaggtgtgtggcccggccctgtca
caacagtgga
agttctcactcaacctgcagcaataagaggcccccggggcccaacgtccacccgcacaccggcccagtttgttga
tcacgtaattacgtttctgaaactttccgcaaaactatatacctcagtgttccatcacggtctgacagtcttaag
tctcaagtcttcatcgaggcatgagcggggcagacagcagtgcagaagaggaaaggagcccctggggctcaggac
agagggtccctcccaaggctggcgccctctgcctccctctttctgcttcctgctcacaccccagcactggccgga
acaagtgggcaagcagcaatcatgtttatatctcagacagaggaagcaggggcaggcgcccccacagtgagagct
cgccaatctccactctgttttacctattcaaaatcgtgtgtgtgtgtgtgaaccaggcatggtggccagtgcctg
taatcccatcactcaggaggctgaggcaggcaaggccctgtctaaggaaaaaaaaaaaaatgatttccacaaagc
tggcctttccttgtctgtccctgcacccgggctcagctggcctcccagccacgtcccctcttccattgacatact
tgctgtatttgaacgtgaactttcctctggtggacactaaatagagataaagagaatcatggtccgttccctacg
cttccccgacatcatcttgtgaaccctgcaacgcagcgcagcccagagcaaaggtgcatctctggggcctcacca
gccggcccacgctgctcctccagtaggggcatccccaaaccactggccccacccacaagcgtcaatgtttaactt
cagcgaggggatcgcagctgggttagaaagtctgctttgtgcccattgtcatctgtaaaactaaataggtcacct
cttttggcaa
gtattgctttgggttttcttttaaaattagatttcttttctttaggaaaactcaaccccacagcttctaactaat
gtgagccagcaggaccctactgccttgtgacagaccagggacactggacagctgtcacccactatccctaggctc
acagtggtttgagaccctgtatgttgggtctttggtactaagctgccggcagagcacagaagagcagctcttcag
cctgtgggtggaccccacgtgcccgtagccctggtgtgggtaatacccccggtacatccacaggcaactgcagga
attatgggtagagttcagatcacaaggcctgcgcactcccactgaggacctaggcatgtgttagccacagaggtg
acctggatgtggggaagtgacacctggtgggctgcaggcatcatacgtgctcatctcgggtatcccagagatctc
gggaatcagagttccaggtggtcagagggcctgtaggtgagtgctgtgcctcccgcctgctccagaaggggggta
gctggtttcttctacctgaggggctccatgtgtgtactcaccatgtgtgtattcgccatgaccatggaccctctg
cgaaggcctggccaggtgctgaccctctgtctttctacccctgccaagcaaccaacagaagagacattgtaacag
acagggtgcccacatgtctaattaagttcatacaatgtccaccttccaatgtaggctctgggaactgtcactccc
aggtacaggtgcaaagctgtcaaggacaaggcaggagtttgctggagtccggtcaccacgacctttgctaaccct
gacactcctgcactgcttctatcagagtgactccacagatgtgagacaacatggggcgtcccaaccacacgtgta
ccccacccca
ccctgtggcctcttaacctttgtaccatctgattgtctggccaccagggtgccaactcaacccagctccgagcag
aggggggtgtgggcagggcccgtcggtgagccatgtttacatgacagtgtgccaaagtgactcgccgagcttgta
ttgctttgtagtcatctgggctatcccaccctcccaactcctgtttttaaaactaaaaactcccaggagccctgg
gcgcacacaggcaaaggtggctgtcttctgtggtggtccttgagtctccgtgaagctgaaaatgatggtgtctgt
gagtatgttttgcaaattcaaaatatagtttggtaattttttttccagttgatttttaaaaaaacaaaacaaaaa
actgctgtacagagcttgtactttgtccattttatagatggaaaccatccttgaaattgtttaacttaaataaag
agaagatactttatagataaaaaaaaaaaaaaaaaaaaaaa (SEQ ID NO: 87) describes UDP-N-
acetyl-alpha-D-galactosamine: polypeptide N-acetylgalactosaminyltransferase
2 (Galnt2), mRNA. Gene name is Galnt2 UDP-N-acetyl-alpha-D-
galactosamine: polypeptide N-acetylgalactosaminyltransferase 2. NCBI
reference sequence is NM_139272.2.
76.
ggcaggcgcactggctttaccatggcggagttgatctctttcgtagttcccacccagagcgacaaagttttgttg
gtgtgggatctgagcactgggcccccagccgaagccttaagtcattctctgttcacagtgttctctcagtttggc
cttctgtattcagtccgagtcttcccgaacgctgcagtggctcgtcctggtttctacgccatcatcaagttttac
tcctcgcgggacgcacagagagcccagaaggcttgcgatgggaaacccctttttcagacatcaccagtgaaggtt
cgtcttggaaccagacacaaggcactgcagcatcaggcctttgctctaaacagctcacgatgccaggaactggcc
aattactactttggcttcagtggatggtcgaaaaggatcatcaagctgcaggagctctccggactggaggatgca
gctctcgctgtgcccatgcagaaggggagcccccagttcctctgcgctgtagaggtggtgctgcccccctacgga
tgcaggagccctggggttggcatctctgaggagcctctgcgccagctggaggaagggcagtcctcgtttcttatg
aagaggaagacagctcagaagcttgccttccaggcggctgtatcagatgccttccagaagctgaccatcgtggtt
ctggaaagtggcaggatcgcggtggagtacaggcccacggcggaggatctagatgccagaagtgaagaagagctg
cagaatttaatccaggtcagctgctcctcttggagtcagtccagccaaagggaggaagaatgtctctcggatttc
agcctggaggaggaagacctgaagctgtgtgatccgcactagcacctctgaccgtgtcaggtgacctgacctgcc
ttctgacttt
gacaggaagttctgcgtgtccaatcagtgggttgctgccctccctccctgagcatccaagtgtgaagccttgatc
ttgctgggctttgtaggagggctctgggggagaggtctaaaggaggcttgagccccgtcactaaggtggaaaaag
gttccccatgcaggcgtcccatcctaataaagactacgt (SEQ ID NO: 88) describes RAD52
motif 1 (Rdm1), mRNA. Gene name is Rdm1 RAD52 motif 1. NCBI reference
sequence is NM_025654.1.
77.
ggccggaagtagtgattgctagcgggggggataccttcgctcagcatggcggcgattcccccagacacctggcag
ccgcccaacgtctacctggagactagcatgggagtcattgtcctggagctatactggaagcatgcgcccaagacc
tgcaagaacttcgcggagctggctcggcggggctactacaatggcaccaagttccacaggatcatcaaggacttc
atgatccaaggcggcgacccgacaggcacaggtcgaggtggcgcatctatttatggcaaacagtttgaagatgag
cttcatccagacctgaagttcactggggctgggattctcgccatggccaacgcaggaccagacaccaatggcagc
cagttctttgtgaccctggcccccacgcaatggctggacggcaagcatactatttttggccgagtgtgccagggt
atagggatggtgaatcgagtgggcatggtggaaacaaactcccaggaccgtcctgtggacgacgtgaagattctt
aaggcgtacccctctggataggtgggatgctgtcccagcactcccaggccccgggagctggtctccggatgacgt
agggtgctgtgtgattctgcgtcatgcatacctactctgaagcagaggggtcgtctctggggacgtttgtattct
gccagacagtcacagacttgaaaagcgggccgcgttgatgagcgcactagctgcgcaccccctggtcacagcccc
gttcagcctccagcgtgagttcttccaggaatatgcacagctcactcgctcttccctctgagcctgagcatcaat
actgctgcttctgacactacacatcccacgcaaagccatattgaattgttgccaaatgaaaaatgcatccaacag
tcaagtgctg
agaaggtgtcaagtggggatgataatgtgtaatgattgggcaatgaatctctgagaggaagcaggaaggagcctt
ggccatttttccccggggagggatgggagtctagtgagcacaaggccgtcacaggcttcccacaggaagtgacat
gctttagggctttagagaaaataggtaagaggtttttgtgcatgatgtccttggttttttttttactatttggat
ttcaagggagtggggcaagaaaggtattaatacctatgtaatacatagaaacttacagaataaaccaccgatgtg
gttg (SEQ ID NO: 89) describes peptidylprolyl isomerase (cyclophilin)-like
1 (Ppil1), mRNA. Gene name is Ppil1 peptidylprolyl isomerase (cyclophilin)-
like 1. NCBI reference sequence is NM_026845.1.
78.
ggcgggacaacggtcggcgacgcagcttgtggtgggagcggaagagctctcccggcgggtctctgtgcag
ttggcgaggcagagggccgccgcagcaccatggcgaccaccgtcagcacgcagcgcgggccggtgtacat
tggtgaactcccccaggacttcctccgtataacacccacacaacagcagcagcagatccagctggatgcc
caggcagcccagcagctgcagtatggaggaacagttggtaccgtgggtcgcctcagcatcactgtggtac
aggcaaaattggcaaagaattatggcatgactcgtatggacccttactgccgtctgcgtctgggctatgc
tgtttatgaaactcccacagcacacaatggggccaagaaccctcgctggaacaaagtcattcagtgcaca
gtgcccccaggtgtggactcgttctaccttgagatcttcgatgagcgcgccttctccatggatgaccgca
tagcttggacccacatcaccatccccgagtccctgaagcagggccaagtggaggacgagtggtatagcct
gagtgggaggcagggcgatgacaaggagggcatgattaacctggtcatgtcctacacgtccctcccagct
gccatgatgatgcctcctcagcctgtggtcctgatgccaactgtgtaccagcagggtgttggctatgtgc
ccattgcagggatgcctgctgtctgcagccctggcatggtgccgatggccatgcccccaccagctgtggc
tcctcagccccgctgtaatgaagaggacctcaaagctatccaggacatgtttcccaacatggacagggaa
gtaatccgttctgtgcttgaagcccagagagggaacaaagatgctgccatcaattccttgctgcaaatgg
gtgaagagtcctagacccgcctgtggcccttggcttcacaccactgacccttggacttgtccacccaggg
ctccccagggtcaccatccccacaagattccaatgaaagaccatccacataccccttccttggacatctg
cgccgccccttcctacatatttcggggatgcatgtggatttttggttctaagcaggcagtcctcttcatt
gttgctgggggacagagttaggggacagttctctcctagtctctagctgggtgatggtggcatggccctg
ttattgcacagatcagtggtcctgtttggggacctgcatctcctgtcctcaccctattctggaaagcatt
cttacagaaagcaactgcctctgccaagatactagaaattgtacagcatccttctgagttgatggatgga
tggcatggagttactcagtccagctgggatggttgttgagggtgcactgagttcatgtgcacttacttac
aggttatttttagaactttcaagctaaaagctccttccctcttctgatgttgtatgtgaacatgggcccc
tcagctagtcctggccagtgagatatgcttcccagccatatccagtgggtctctaacctcctgtctagct
tatctgccactgtctggctaggcttcagccacagaaatccaacttgaatacagaaggtatccagacctgg
tgcttggttggccaggccccacattagccctcactgctggaggctgctttccacatcctcttgtttagta
ccagcatcatgggtcttactccaggggctgttcttgttgatagaccttcatcacagagcccagagtcaag
cgaaggagatgggtgggtgtggcactatgtgcttactactaaaattcaagcattgtgacactactgacct
gcagtatactctgtggacccatactgtagcttggcttgagtgataaaaattcatgcttaaattaaacttt
cataaaggtagaggtgcctgtacctgaccagtggatattgtatagtgtattttaggttgcaaacctttga
gcccgtgacctggccatacatgtatttagggagaaattgagtggtgccttgtgctaagatacagaccgtg
aaagctttctcagaaccagtactgtcacaaggtgctgttaggaagctccagcattgccacagggttatgg
tgggacagttttgccttacattgccgttactttttgttttctgagaatctgcacacccggtttggatatg
tgatccaccaataagggcatgaaggtgactcctagcagtttggactgttgtgaaggacttcaaataagaa
aattagtgcttcagtttttggtctttgcctcctaaaaacgcggtaaaatatcctacttgctgttatctac
aggggtgctttttgtcaaatgtattgtttataagtatttatttttgtaagcctaaaggcagtatgtggtg
atggtgagcatgggatggtggtaacaagcacttttctttagttgtaggtctttaaaccttttaactgcaa
aatggagaagaaagaccaatgtttattaaatagattcaaccacttactttaacacaaccaggcactggaa
gcctccattctagactgttgatcggccttgtgcttggtaagcagatgccctcttgcagggtggatgagtc
aggcaggcagaacctccttaagtggtccacttagtgttaccagcagactgcagacactgcagagcaggaa
cctcactcagtacctgtctctaggtctcaggtgtggcggctgtgtgcatccgcatctgccttacggaggt
ataggttgaacacagggccactttgttctgtgaggttctataggaagcatcacgatttgctgccagcccc
tgagcagtcccacagcactcagaggcagcgttcactaacttacgactgcttaaatgaggaagagtgatgg
ttttaagacagatttaagacttgagctgaacacagactcttccctttgtcatgaacattttattttttta
atagattgaagaaagatcacattttataaggcctgagtgatactcctaggttttgctgtttttttgtctt
ggaatttcatgtcagcgcccatctagtgcaaggagagaggcagcacttgtcagcgcctgtgtaggtacca
gggcatcagagcatggcttgttggcttccatcccatcctgaatcttgttgagatgaagagtcatccctag
aaggagctcagaggcagaggccacagccgagtcagacaggcttctgcatggggaccaggctctcactggg
tgtaattgctgctcttgccaagtttgaagattgatgttggtagcaaaatttctcaaggtgttcactggag
ccccctcccctcgggccagctctagtgtaggcatgcatctagggatggcctggcctgttttcttctctgg
acatgttggttggctgaagttgtaacctgctgcacgcttgtgggctgacatggccagaggctttcatctc
accatgtgagtgatgcctacccgatgacagaagtgttgtgcgactgggctctgccctcgccctcccaggc
caccctttacagacactggtatcatctggtacactgtctggaggttggtgggaatggttctcagaactgt
ctgtgattattttgggaatgggggggtccatgtccatatttttctttgattatatgatatatttttactt
ttcagccttctaatttaataagtggtctatataaaatagaaaaataaagtcctctgaggaaatgtt
(SEQ ID NO: 90) describes toll interacting protein (Tollip), mRNA.
Gene name is Tollip toll interacting protein. NCBI reference sequence
is NM_023764.2.
79.
gcggcccagcccgccacggaggcgcgcaggcgggcgcgcggagtggccgctcaggagtatctctttacca
cccccaagaaactactcaatgctgaagcccttcaaagtgatagcggagactttggaagcaccatggatgg
gttttgtgatcagcaagtcccttttatggtcccagggaaatctcgatcagaggactgtcgagggcgacct
ttgattgacagaaagaggaagtttgtggacacagatctggctcacgattctgaagagttgtttcaggatc
ttagccagctgcaggaggcttggttagctgaagcacaagttcctgatgatgagcagtttgtcccagattt
tcagtctgataacttggtgcttcatgctccacctcccaccaagatcaaacgggagctgcacagcccctcc
tccgagttgtcgtcctgtagccacgagcaggctcttggtgctaagtatggagaaaagtgcctctacaact
attgtgcctacgataggaagcctccctctgggttcaagccattaaccccgcccgccactcctctgtcacc
cacccatcagaattccctatttcccccacctcaggcaactctgcccacctcaggtctcacccctggagct
ggcccagttcaaggggtgggtcctgccccgactcctcactcacttccagaacctggatcacagcaacaaa
catttgcggtcccccggccaccacatcagcccctgcaaatgccaaagatgatgcctgaaagccagtatcc
atcagaacagagatttcagcgacagctgtctgagcccagccatcccttccctcctcagtcaggagttcct
ggagatagtcgccccagctaccaccggcagatgtcagagcccattgtccccgcagctccgccaccccttc
agggattcaagcaggaataccatgacccactgtatgaacatggggtccctgggatgcctggtcctccagc
acatgggttccagtcacccatggggatcaagcaggagccccgagattactgtgctgactcagaagtgcct
aactgccagtcatcctacatgagaggcgggtatttctccagcagccatgaaggattcccgtatgagaagg
accccaggctgtactttgatgatacttgcgtggtacccgagagactggaaggcaaagtcaagcaggagcc
caccatgtatcgagaggggcctccttatcagagacgaggctctctgcagctgtggcagttccttgtcacc
ctcctggatgacccagccaatgcccacttcatcgcctggactggccgaggcatggaatttaagctcatag
aaccggaagaggttgctcgccgttggggtatccagaagaatcggccagccatgaactatgacaagctgag
ccgctctctccgctattactatgaaaagggcatcatgcagaaggtggctggggaacgctacgtctacaaa
tttgtgtgtgacccggatgcactcttctctatggccttcccggacaatcagcgcccgttcctgaaggcag
aatccgaatgccccctcaatgaggaggacacgctgcctctgactcactttgaagacaaccctgcttacct
cctggacatggatcgctgcagcagcctcccctacaccgaaggcttcgcttactaagtttctgaatggccg
agtagccacaccctagagctagcagtcctcatccaggcaaccgagggcagtggttttgtttgtgttcttg
gctgttcctaaagcttgccctttgagtattatcaggagaacccaagctgtctctggattcgacacccttt
aagacaaataccttggctcgggaggagtgggaacagggagagacaaaaaccaccacttcctggggagacg
tggataccgtcctccttgccaccatggccagtatgtgcagagcagtactgcagtgtgttcagggttagcg
cccatgaaaggggaccgagtgtatgacaatctgcgttgatcatggactactaaaatgccttcacacagga
ggctttgacttgcacaattctttgggaagaaactgcacacccataggacagtcgataaggctgagtcggg
gaggctcagaccattgaggcttgaaattgcaaaactcttgtagctcattctgaaatctttcccctgtagt
ccagaactggggaggccaggagagccgttactcactccatggctctcagaccgcgttcctctttggcctt
ccttccagccgagccgtcaccataagtaaatcaagtcccttcctcccagtcacacggctgagcgcagacg
gcttgttttaaaaagatgatgaacatgacagtattttgttttgctttgatttgggggggaagggttcatc
ttgcttttggaggttgttttggggaaaaaacagtttatataaactgatttttgtagttttggtatttaaa
gcaaaaacaaacaaaaaacaaacccctttttggtacctgcactgcatccctcgggtggggcagtgcctac
tttggaagatgctgcagcctgcgaggggatctgctggggcttccccttcgccacacagaagcctgcttag
tgctttctgcaccagacaacctgttgaggcgtataacctgatctgtacatttttttaagtgtgcagggca
gcctggacacagcttagcgttctacgtgtatagttctccatgctcactaactctccttctctgggaagca
gatgtacatactacatacatgtcacatactacatacatgtcatgtactacacacatgtcatatagtacat
acatgtcatgtccatttaacgactggtcacctgctggggacccaggcatctgccttgttttcctttttct
ttcagttttggactttggaccctgcacagccgttccccagggtctgaaacttacctgtcacgcacaggta
gagcagtgctagctagcattactggcttcctgctactttttcgtttttttaaattttttattctgattgc
tgtattatattggggatgttctaaacaaccaaactaaagctgtgtacagattgggtttggagcggtgggg
acgtcagcaggaaacgccagtgctttgctgcctgattgacaggaatttgctgtccttgacactccacaca
ttgtgttcctcacgtgtgggacaggacacagagctgactgagcgtgggacactccacagccttctcatga
cttgtgaaggaggttcacaggaacgaggggtatgttttagagctatgagaacctagtcagcccacagagg
cgtcaaacctgaagcttgggccttaattagcattgtactctgatgaaaggactctttcataagcatattt
atagctttctaatctatacacagtctatccatagatgcatattttaccccaactggctagagatttattt
gttgtaaatgctgtatagattttgggttttttttttttgttcttcctctcttcacttatactggtgtggg
gtttgttttagttttctttagtgaatttatgctgttttggggggcggattgagaagaatcttccgtagcc
tgagctgtccctcctgttttgacggcacatgtgccagtcaagggacatgggacagtcgcgccactttgca
ttcccacgggactcatgtacctcccgcagagccgtccccgtcaaggtgtggggctcctttcatgcatttg
cttcttttccagagtgtggatttgaactacttccccctgcaaagggccgcgtgcaccctgccgagctgcg
tatattctgtactgtgtacaataaagaagattgctttccttaaaaaaaaaaaaaaaaaaaaaaaaaa
(SEQ ID NO: 91) describes ets variant gene 5 (Etv5), mRNA. Gene name
is Etv5 ets variant gene 5. NCBI reference sequence is NM_023794.2.
80.
cttttctctccgagcaagatggctgggggtgagggtggagtgactctcggtcagccccatctttcccgac
aggatctcgccacattggatgttaccaagttgacaccactttcacatgaagttatcagcagacaagctac
aattaatattggtacaattggtcacgtagctcatgggaagtccactgtggtgaaggctatttctggagtt
cacactgtcagattcaaaaatgaactcgaaaggaacattaccatcaagcttggatatgctaatgctaaga
tttataaacttgatgacccaagttgtcctagaccagaatgttacagatcgtgtggaagcagcacaccaga
tgagtttcctacagacattccggggaccaaagggaacttcaagttagtcaggcatgtttcctttgttgac
tgtcctggccatgatattttgatggctacgatgctgaacggtgcagcagtgatggatgcggcccttctgt
tgatagctggtaacgaatcatgccctcagccccagacttccgaacacctggctgccatagaaatcatgaa
actcaagcatattttgatcttacagaataaaattgatctggtaaaagaaagtcaggctaaggaacaatat
gagcaaatcctcgcatttgtgcaaggaaccgtagcagaaggggcaccaattattccaatttctgctcaac
tgaagtacaatattgaagttgtctgtgagtatatagtgaagaaaataccagtgcccccaagagactttac
ttcagagccccgtctcattgtcatcaggtcatttgatgtcaacaaacctggctgtgaagtcgatgacctt
aagggaggtgtagctggtggtagtatcctaaaaggagtgttaaaggtaggccaggagatagaagtgagac
ccggtattgtctccaaagacagtgaaggaaaactcatgtgtaaaccaatcttttccaaaattgtgtcact
ttttgcggaacataatgatcttcaatatgctgccccgggtggtctcattggagttggaacaaagatcgac
cccaccttgtgccgagctgacagaatggtgggacaagtgctcggtgcagttggagctttacctgaaatat
tcaccgaactggagatttcctatttcctgcttagacgtcttctgggtgtacgtactgaaggagacaagaa
agcagcaaaggtccaaaagctgtctaagaatgaagtgcttatggtcaacataggctccctgtcgacagga
ggaagagttagtgcagtcaaggctgatttgggcaaaattgttttgaccaatcccgtgtgcacagaagtag
gagaaaaaattgcccttagcagaagagttgagaaacactggcgtttaattggttggggtcagataagaag
aggagtgaccattaagccaactgtagatgatgactgaagacttcaagttaaacaatgccttgggaaggag
ttggaatttcttttactagcctaggggtatattttcaaagcaatactagaaagaattaatttcctagctc
attaccttactagtagcagctataaatttattcttatattttggcgatgaaaatgtgacctctacttttt
tttttctttttattgattccttgtttgacattatgtagcccaatcccattcatcccctcatatccaacct
ttgcccatgcgacctcccccaaagtagcacaccaacaacagaacaaagcctagaaaacatgccatcgtgg
aagctgtagcttctgtgacaacaacaatgttggctcctcatcaggactccctccagttatcctgttgctg
cgctgtgtcatagagatcctgcagctttggaacagcagggtggccctttcacgatccccaccattcgaac
atgacatagattttaggtgggtcaattcagagccccggataagggccaggatggtagctgagctggtcag
ctggggcccattcttccaagtgctgcggcttgtgagagcctgggccaattctccctacagaaggtggcaa
cgggggagggtgtcccatccacaccacctcataacagacaagtggcagggccagctgtccagagtgctat
atccaatgagggatggtggcagacctgcacagcctcagacacccagtgttccctagtggtaataatgagc
catggacactgacccaccactgtggatccacagactcaaacaaggccctgatggcagggatgcccactta
taacaggctacttctctccacccttgaattcctttttctctcccgtctgtcatggtgacaagcaggtatc
tatgcttatgcactggagggcaggactgtgggtggcatggaggtctacaagaccttgtgttcctcttctc
atgccgtgctgccttgctttaatttgatttttatgagtcctaggcataagacaaggtttggccatcaagc
taggatgaacaaaggactgccatctgctctgcccctgataaagaacaataccaccaataaggggtctctg
ctggttgctggggttgtaacctacttctaatatggggcttaaaattaaatgtgaaaagtggtatactata
tacattggatcaagtctatatagtagctaaaattaagcattccaaagtaatgtctaatttatcaatcaat
gaaagtttgaagtgaaacttggctgcaaccagtctttggcgtagcacttgtatcatgaacctgtctaaga
tgaaaatgatctcttctcatgattcatctgtgagtagctccatagtgaagggcaattagtaaaggctaaa
gacttacccctttatttgtccttctatttggggttctgttgtctgaaatttgaaggaatacgaccagtgg
gctcaaatgagcaccactcttagaagttgtgtgcccattcccttaacacaggtagatctgtcttacccgt
ttcctgggtgggaccttaggaatccagtgccctgagactatgtctttattatttatgcctagtatctcag
tcttcccacactggcaaagagtatagcaactggtggttagtgataattgacaaggttctgtccaggctgc
ttttttgaggtctttgttttagtaagctatgaatggagctgtctcttgcatgatgaataaacactctacc
attgtctccactgcccctagctgtagtactggctgataggattatggtggaaattgtgttattggtaatc
tttgagccttgagagaggtagaaattgttcagaaactgtgtcctctggtttctaaatctattccccacgt
gcctccaattttcttgtgctgtgcattttaaattaatcatgttttttcttaattaaatactctttcacag
caatgatttccaactctggctgacgtaagaattgctggggaagtttgaaggggggaaaaagaaagctctg
gggtggagtccaggctttgatagttttttttgtttgtttgtttgtttgttttttttttttgaggaaacct
attgatatatgtatttgattgactctaaaaagttgttgtattggaacatgaagaaaacatttaaataaaa
caagattctgaaacaagaaaaaaaaaaaaaaaaaaaaaaaaaaaaaaaaaaaaaaaaaaaaaaaaaaaaa
aaaaaaaaaaaaaaaaaaaaaaaa (SEQ ID NO: 92) describes eukaryotic trans-
lation initiation factor 2, subunit 3, structural gene X-linked
(Eif2s3x), mRNA. Gene name is Eif2s3x eukaryotic translation initi-
ation factor 2, subunit 3, structural gene X-linked. NCBI reference
sequence is NM_012010.2.
81.
gcgcactggtaccgacttgccgcgcgccgccgctcctctttccttcctcttccgagcacgtcctgctccg
tgtacctctcgggtccctcgccatggctgcgctcacccggaacccgcagttccagaagctcctggagtgg
caccgcgcgaactctgccaacctcaagctgcgcgaactttttgaggcggatccggagcgcttcaacaact
tcagcttgaacctcaacaccaaccatgggcatattctggtggactactccaagaaccttgtgaacaagga
ggtgatgcagatgctggtggagctggccaagtccagaggcgtggaggctgcacgggacaacatgttcagt
ggttccaagatcaactacaccgagaatcgggcggtgctgcatgtggcccttcggaaccggtccaacacac
ccatcaaggtggacggcaaagatgtgatgccggaggtgaacagggttctggacaagatgaagtctttctg
ccagcgggtccggagtggtgactggaaagggtacactggcaaatccatcacggacatcatcaacatcggc
atcgggggctctgacctgggacccctcatggtgactgaagctctcaagccttactcgaaaggaggtcccc
gtgtctggtttgtctctaacattgatgggacccacattgccaaaacactggccagcttgtcccctgagac
ttccctctttataatcgcctccaagaccttcaccacccaggagaccatcaccaatgcagagacagcaaag
gagtggtttctcgaagcggccaaggatccatctgcagttgcaaagcactttgtcgccctgtctacgaaca
cggccaaagtgaaagagtttggaattgaccctcaaaacatgttcgagttctgggattgggtaggtggccg
ctattcgctgtggtcagccattggactttccattgctctgcatgtaggttttgaccacttcgagcagctg
ctgtccggggctcactggatggaccagcacttcctcaagacgcccctggagaagaatgcccccgtcctgc
tggctctactgggcatctggtacatcaactgctacggctgtgagacccacgccttgctgccctatgacca
gtacatgcaccgctttgctgcctatttccagcagggtgacatggagtccaacggaaagtacatcaccaag
tccggggcccgtgtggaccaccagacaggccccatcgtgtggggggaaccagggaccaatggtcaacatg
cattctaccagctcatccaccaaggcaccaagatgataccctgtgactttctcatccctgtccagaccca
gcaccccatacggaaaggtctgcatcacaagatcctcctggctaacttcttggcccagactgaggccctg
atgaaggggaagttgcctgaagaggccaggaaggagctccaggctgccggaaagagcccagaagacttgg
agaaactcttgccacacaaggtctttgaaggaaaccgcccgaccaactctattgtgtttaccaagctgac
acccttcattctgggggccttgattgccatgtatgagcacaagatctttgttcagggcatcatgtgggac
atcaacagcttcgaccagtggggagtggagctggggaagcagctggccaagaaaattgagccggagctgg
agggcagctctgctgtgacctcccatgattcctccactaacggactgatcagcttcatcaagcaacagcg
ggacaccaaactagaataactccagccgcggccctactgactggtcctccgtgtcccttctcaccatatg
cactgcatggtcctgcccctccctgcccagagcgcaccaccggtagttggcctggactacaaggctgttg
ggagaagctggtctggaactgccatccacccactacgcaccctccctgttgaagctgatggaagggcttt
gacgtgtcatgttgttctgacctgtatttcacaccccagctagaataaagacacctagaggaggcatggg
ggctcagcctcttcagtttactgagtcagtacttttatctgttacttgtgggtgaccctgcttatgcctt
gtttggttggctctttacttggtggtgggctggtccacagtctcggaggattgatcgttatgaggggaca
gctgggtgataggggcacttgggaggcacaggcaggtctatctgaattcaaggccagcctggtccacaaa
atgagttcccggatagccagggctacacagagaaaccctgtctacaaaaaaggggttgggtagggtgggg
tcagggatctgcagttaggtgggcaacttgctctccagtcacgtgttcacctccttcccctcctcacctt
ggtgacgtggaaaaagtaattctgcttcactaggtaggtttttgcttgcttgtttgtttgtttaccttcg
ctttatttttggtatcaaaacctaggaatggggctggagagatggctcaacagttaagagtactgactgc
tcttccagaggtcctgagttcaattcccagcaaccacatggtggctcacaaccacctgtaatgagatccg
atgccctcttctggtgtgtctgaggacagctacaatgtactcacatacataaaataaataagtcttaaaa
acaaacaaaagaacccagggatgaactcgattatacccttcaaaagacagttttagcagggtgtcattac
tgtcacattcatatcctggctatcagtgagtccttgtgagactgagtgacagccctgtcattctactttc
caccatgttaattaggcccatttgatggccagtcaataaaaagacttggtacatgt (SEQ ID NO:
93) describes glucose phosphate isomerase 1 (Gpi1), mRNA. Gene name is
Gpi1 glucose phosphate isomerase 1. NCBI reference sequence is
NM_008155.
82.
ccaggcaggggtcgccgcagcctggatgcgccctgtggcgcgcgcgcacgcagcatcccgagcctcccgc
cgcgcggcggggatgcctgctctccgagccccggggctgggcccccggcggtaaccggagcgggggggcg
cgcccccccagcagcagctgccgcgcccgcgcccgggccagtcgccgccggggcccatcgcctgtcgccg
cgctctgcgcacccaccgccttgcgcggccatggggacgctgctggctctcgtggtgggcgcggcgctgg
tatcctcagcctgggggggctgcgtggaggtggattccgataccgaggctgtgtatgggatgaccttcaa
aatcctgtgcatctcctgtaagcgtcgtagtgagaccaccgccgaaaccttcacggagtggaccttccgc
cagaaggggacagaggaatttgtcaagatcctacgctatgagaatgaggtgctgcagctggaggaagatg
agcgctttgagggccgagtggtgtggaacggtagtcggggcaccaaggacctgcaggacctgtccatctt
catcaccaacgtcacctacaaccactctggcgactacgaatgtcacgtctaccgtctcctcttctttgat
aattatgagcacaacaccagcgtcgtcaagaagatccacctggaggtggtggacaaggccaacagagata
tggcatccatcgtgtcagagatcatgatgtacgtgctcattgtggtgttgaccatatggcttgtggcaga
gatggtgtactgctacaagaagattgctgctgctacggaggctgctgcacaagagaacgcctcagaatac
ctggccattacatccgagagcaaagaaaactgtacaggcgtccaggtggctgaatagcgctggctctggg
ctccgcctcatggaagagccagcccctacgggtaccctcccgccctgcagtggggatcagcccctggtgg
gtaccctcccccggcagtggggatcagcccttcggtctccccagcctcacagttctgcagtggagccacg
agggtgggagcgggcagggactgatcccacctcacccaccgcctcccacctaccctcccaccgccatgca
tgatgggtgaagcaatatggccgccccaccctgcttttgctgcctgtttgggggagggggcggtgaggcg
agggggcaggccccgccccttctttttgctgatttgcacataggccacttcccacacgcactgccaggcc
agccggccccacccctgcttgatggggtcaagaggggtcgggacagggacagtagtgggcaggaggttct
gggcctcatctcccctccgcttcctccggctggacctggggttcccttcctgtgacacctcctagccctg
gcccacccgccctctctcaccagccttcaattgtggtctcttgggaagggcctcttcggcctcctctctt
tacagaagtagtttttgttcatgaaataaagattcttggactcgaaaaaaaaaaaaaaaaaaaaaaaaaa
aaaaaaaaaaaaaaaaaaaaaaaaaaaa (SEQ ID NO: 94) describes sodium channel,
voltage-gated, type I, beta (Scn1b), mRNA. Gene name is Scn1b sodium
channel, voltage-gated, type I, beta. NCBI reference sequence is
NM_011322.2.
83.
ggaaggggccggggcagctccggggaagggaagggctggagctacggcgtccggcggccgagaggggcgg
tgggctgggtgcgccgcggagcccggcccgagtgtcccgtgcagcgtccccgggtgccgccatgacgccc
gatctgctcaacttcaagaaaggatggatgtcaatcttggatgagcctggagagtggaaaaagcattggt
ttgtgctgacggattcaagcctcaaatattacagagactccacggctgaggaggcagatgagctggacgg
tgagatcgacctgcggtcctgtactgacgtcacagagtatgcggtccagcgcaactatggcttccagatc
cacaccaaggacgccgtttataccctgtcggccatgacctcgggcatccggcggaactggattgaggctc
tcaggaaaactgtgcggccaacttcagccccagatgtcaccaagctctccgactgcaataaagaaaacac
gctgcatggctatggcacccagaagagctccctgaagataggggagcagcggacaggctctgaggttatc
ggccggggtggtcctcgcaaggcggatggaccacggccgtcactggactacgtggagctctctccattag
cgccaagctccccacagcgcatgcgcactctctcccgctccactcccgagcgccccaccaagcaggagga
tctggagcgggacttggcccaacgctctgaggaaagacgcaagtggttcgagtccacggacggcaggacc
ccagagacgcccagtggcgatgggtctcgaaggggcctgggtgcccccctgactgatgaccagcagagca
ggctcagtgaggagattgagaagaagtggcaggagttggaaaagctgcccctgcgggagaacaagcgagt
gccactcaccgccctgctcaaccaagcccacaacgatcgtcggggacctacaagtgacagccatgaggcc
ctggagaaggaggtccagtctctccgtgcccagctggaggcttggcgtctccgaggggaagctcctcaga
atgcaccgagactccaggaggacagccatatccccccaggctacatctcgcaggaagcctgtgagcgcag
cctggcagagatggagtcctcacaccagcaggtgatggagcagctgcagcggcaccatgagcgagagctg
cagcggctgcagcaggagaaggagtggctcctggccgaggagacggctgccacggcctcagctattgaag
ccatgaagaaggcgtatcaagaagagctaagccgggagctgagcaaaacccggagtctccagcagggccc
agagagccttcggaagcaacaccagctggacatggaggcgctaaagcaggagctgcaggtgctatctgag
aggtactcgcagaagtgcctggagatcggggccctgacaaggcaggcagaagagcgtgagcacaccctgc
ggcgctgccagcaagagggccaggagctgctgcgccacaaccaggagctgcactcccacctgtccgagga
gatcgaccgtctgcgcagcttcatcgcctcgcagggcacaggcaacagctgcgggcgcagcaatgagagg
agttcctgtgagctggaggtgctgctgcgagtgaaggagaatgaactccagtacctgaagaaggaggtgc
agtgtctccgagatgagctccaggtgatacagaaggacaagcgcttcacggggaagtaccaggatgtgta
cgtggagctgaaccacatcaagactcggtcggagcgtgagatcgagcagctgaaggagcacctgcgcctg
gccatggctgccctgcaggagaaggaggctgtgaggaacagcttggctgagtagaggtccctgtggttcc
gatcagctgaggaccctccagccccaggggaccagccaccctccttctctggcagaagtcagaagccaag
ctttctccccaccctgggtcacatgtgcactcacaccacacacacccacacacacacgcacacatacaca
cacacacacacacacacgcatatgcacacacatgcacacacacatacacgcacacacatgcacagacact
gcggatctgagctcgtcccttgcacagggtctttaccttgcaccttcttcaggattttatatgtgaagag
atttttatatagatttttttttttgcctttttttttttctaaaacatttaaactttttaaaaaggcaatt
tcttggtggcgtgtgcctcaagcactggcttctctccctcttgtcccctgcttggccctccttcctacct
gggtgtctcacaaaggcccaaggagagccagggtgagtcaaccagagggaaaaggcagccctcagttccc
tgccctggcctctgggcagagggaggagccagtggctggccccatatccactcctctgaggggcgctggg
cgtgggccaccagcgccaccagcgccacccatgaagacacttctcttctgttccgtgagtcaccctaact
taataaaaccttccggcaaaaaaaaaaaaaaaaaaaaaaaaaaaaaaaaaaaaaaaaaaaaaaaaaaaaa
aaaaaaaaaaaaaaaaaaaaaaaaaaaaaaaaaaaaaaaaaaaaaaaaa (SEQ ID NO: 95)
describes TRIO and F-actin binding protein (Triobp), mRNA. Gene name
is Triobp TRIO and F-actin binding protein. NCBI reference sequence
is NM_138579.2.
84.
tggggtcagccgccgcaacccgggggaggaggttcccactttaagaagtgaagttttcctccccctcccc
ctcctgcccacctcctgccgcctcccgcgccccgcggggccgcggatggagctccgccgtggcggcgtgg
ggaaccaggctgcgggccggaggatggatggggactgccgcgatggcggctgcggcagcaaggacgccgg
gtcggaggactacgagaacctaccgaccagcgcctcggtgtccacccacatgacagcgggagcgatggcc
gggattctggagcactccatcatgtacccggtggactccgtgaagacacggatgcagagtttgaatccag
atcccaaagcccggtatacaagcatctatggcgccctcaagaggatcatgcacactgaaggcttctggag
gcccctgcggggcctgaacgtgatgatgatgggtgcagggcccgcgcacgccatgtattttgcctgctat
gaaaacatgaaaaggactttaaatgacgttttcagccaccaaggaaacagccatctagctaatggtattt
tgaaagcgtttgtctggagctgggaagctcttctctctggggcttcctcccccggacccagcaaccttca
tcctcggcaaacagagaactctagggtcacgtgaatgcggtcacgtgaacgcgcgctttttttttttttt
tctttcaattcaatccctctttctctgggggaagctggtggtagatgtgttttggtatttggtatttatt
attgtgtggcttttttttttttcttttaagaaaacactcgaaacaattaatgatcagtcataggaaaaaa
tatcttttgaatgctggatgcaaaccaaaaatacacagaaaaggacagcttctttgagactatttaaaaa
ctggtacagcaggtatctccagcgccaggatctaactaagctttgaaaggtcaagacgttttatgctgac
agaggactccgagccactgtggaaggccatccccactttaagctgccggagactcgggcctttgaccccc
ccccacagcgtcctccgtgtgtctgagtcgcatctttctgcaagccagggctgaactagtttcctgtggt
tgatctagggagggcgggtggaccgcaatggggcacttgtggacagcttttacataaggcaggagtcgat
gggctgggactttaacaggcttttcagtgggtgggagaggaccgtcgatagcagcaaagcaacttaaaat
aatgtttattttgacaaaatttactctgaggaatggagaagtggatgtgatcaactctcccctttgcttt
ttattcgacccacaataaaaacggtttcccttctgtcagccacccttagacccaagcatacccgggtgct
ttgctgaagtcctttgctttttcatttggtgaccattctttgaatttattcctttggtcaccaagagcca
tgcccccatgcttctgagatggtggtaacagaccctcatttcacccaaactaccttacgtaatggcttgg
ctcctttaaggaggaggctaggggacttgacaaaacccatccctgcaccagggtctgcccaatgctgtcc
ctgctgcattggtgtaaatgctgtggtgtctgccacctttctgggaataattgtgtgtgtgtctatgtgt
gcactaagtaagaggtgccttcctgagaggtggcctgtagtctgacgctcatccctccctctttccacct
gttataattaacgggcagctgcattgctgtggaagtcactgggaggccagaagcagaaattccagggcca
attcaggctttcaaagccaggctaaaggagacttcagatgaaattcttcacttacaaggctggtcctaag
tctgcttagacttcagctaaggcaactagccttctgcatgattgtatcctcttcagtagccttagcttaa
gggcccagagagctctctgtacctaaaggcttgggacaagggaataaaattctaggcagtgggaaatgcc
tgaggtatggtttctgagctctaacttttcccaaggttgatttacaaacaaaaggagtctctgaggttta
gctgcaacggtgctccagatagctgctctccctattgctgttcagcgtttctgccaccactttgggattt
taaaattttctcaccttggctgagctgctaggcaactttgagccttagaaagctaagtccagagtttatc
tgacttaagttcatggaacttcccatgagcaccaggtgacagcgggtaattttctggagtttgttttgca
ggggtagctgggagtatggccaccctactccacgatgcagtaatgaatccagcagaagtggtgaaacagc
gcttacagatgtacaactcccagcaccagtcagccttcagttgtatccggacagtgtggcggaccgaggg
gttgggggccttctacaggagttacaccacacagctgaccatgaatatccccttccagtcaattcacttc
atcacctatgagtttctgcaggagcaagtcaaccctcgccgggactacaacccacagtctcacatcatct
caggaggcctggccggggcactggccgcagctgccaccaccccgctggacgtctgcaaaaccctcctcaa
cacgcaggagaacatggctctctccctggccaacgtcagcggccggctgtcgggcatggccaatgccttc
cggacggtgtaccagctcaacggccttgccggctatttcaaaggcatccaggctcgagtcatctaccaga
tgccttccaccgccatctcctggtccgtttatgagttcttcaagtacatccttacaaagaggcagctgga
gaatcgaactctgtactaaaggaatggggcagtgggaagtggttctgacatcctttgttccagcttcatc
ctcttgttctcgcacccagggcttgtcctgaagggtgcttgcgccaggcccttcgttccctggtgccgta
gagagggaggaccggcctccactgaccagcatgctgtctgtggggacatccgaggtggtggtaggcggga
gaaacttggggagagggagaagttgcttttctcctccttcctccagaggaatgtagcttttctgcctcac
tgtggccttctccctagatctttagatcacaccgcagaggcaaacagtgacacagcaaagaaaaagttta
tgtacataaaagtaccattaccccagtctgaaataggtggataatgttatcctttatttttctacataga
gatttttgaaattgtgtgtgcttgtgtgtgtctataaataatattagagttgggaatgatggacctgttt
tgtttttttgttttttaaataagagagttccattcttccatcatgagaagtaaaacaaacaacaacaaca
acaaacaaggggaaaaaggcaccaaagtaataaatggctttatgtggcctgaaattgtgtgtagcgcccc
tcccatgggagtttaacttgaatgtaaaataataaatataaagtttatattttgaatttctcttttcatg
gggaaaaaggtacgttgaaaacaaatcaaaatagtctatcacttggagcttttttttgttttgtttttaa
ttttaattttaaatacagttaccctgcgtgcctttctgtcttttctaagacttccaggaccaaaattagg
gtcctggagcattttctccctctgtagacacccccgtacagtcctcagcaccatggctgtctcagtagaa
taagaatggctggccttaaccttccgtgtgagtgatgtgagcgtgtgcctccctgttcagctcttagcac
tgggaatgccactgtatgcctagccaattgatgagacagttcctgcctttgtcttaggagtaatctccat
agcaacctcctccttgggcacttctgctaacctggtctataaggtttgttagtagtctcctgttgaccca
tttccacggcagtctagggattttgagtatttctggtactcatctgtcacctaccctgttcagctccacg
acttaaacatcagagcctcttcactgcactgaggccagccctccagccgggactgaagcctgccacgatc
ctccgcccctcccgagctgctcagcacgcttgtctcaccctgtgtcacttgagaacctccgtcctttccg
ctttgttttctgaagtggagtcagatcatctcagagaaagcacaagcctgtgttgtccagtctcccatcc
tcatggagatgacaggataggctcagtttccttcttccagttgtcaacgagtaagctgggaagacgctca
tgttttcgtgtccattgccctggtctcatttgggagccacgactagcatgctccctcactggggaggctt
agaccttgagttcccacggatggtcatgctgctgcctgggaagaaattgagcttgtacttggagaatttc
atcttggacccacgtaccacttggtgccagtagatgcagctttttggttttgtccagcagctatgagaag
ttacacactggtcagctgactttttttttttccccagagagaaccacctctctcaagtactcctatgtat
tccaggagggcttcctggctggctgcagtttgtcttactagtgtcctgggctggctggtgtcctattatc
actgacaatgtttctattcttaccctgctagcattgagtagagttaaattctataaatggagagggaagt
caaattgtagagtctaaagtcaaatttaaatcaaaccccaaactattcctataccacgcttagatacatt
gttgactaatcctgctgcaagaccaatattttcccttggattttctttacagttatctaatactaggata
gcccctttggtcatggccttataaggtaggaccaggacttcaggactctgtgagcttgaagtccaggggt
cccatcctggctatcctctcctcatgattggtgggtctgcaggaagatagcttttttaacctgcccatcc
aattcagtcaccctaaggttgagcgagtgatatgtctgcaaaaaagttgttaaaggttgggatacattca
acgtaagttctgtgtcagtcagtcccccctcccttagctccaacattgaaattgtccttgaccctcacgc
acatgatagccagtgtgttgattgtcatttttgacgttgcaaatttcaagataggaaaaggctcagtgac
cagtttaggatagccataacctatctcggtcattggcatctttggagtgacatccaaggatggaccatcc
cacctgtgtacaatgataatactttagctgtgtgtgtgtgtgtgtgtgtgtgtgttcaggccatgtgtca
gtcaaccatttaatagatgagtgttaatttattatagataaatattgatctttaaaaatgggatgctctt
aattcatgactttttaaagaattggattaaatatagctacttttatgtgagctttcccctctgtccccct
gccccttctctctgtctcatttctcacaaccatcaccctccagtgtcacccccaaggctatatctccaac
atactgctatgccattaaggttttatttaactttatttatggataattatgtctatgaacaacaaatgtg
ccgttaccggtcacacagtgtgtgggataagagtggcctagtgttgctttccgaatgtcttaaaatacct
gtggtcaagattgtttttgcctttgtggcctttgggtttggagagcagcattgtgttcacatgtgtggat
gtctgtcgtgctgctacagttgtaacactggagctggaaaaaataaagcattgatcttg (SEQ ID
NO: 96) describes solute carrier family 25, member 37 (Slc25a37),
transcript variant 2, mRNA. Gene name is mitochondrial solute carrier
protein. NCBI reference sequenceis NM_030054.2.

What is claimed is:

1. A diagnostic marker for brain disease caused by brain injury, comprising one or more genes selected from 90 genes listed in the following Table or protein expressed therefrom:

| | | | GenBank |
|---|---|---|---|
| No. | Probe ID | Gene Name | accession # |
| 1 | 299424 | unknown(Sequence No. 1) | AK033066.1 |
| 2 | 305559 | unknown(Sequence No. 2) | |
| 3 | 328759 | fibroblast growth factor 20 | NM_030610.1 |
| 4 | 354854 | RIKEN cDNA 4930451A11 gene | AK015436.1 |
| 5 | 355052 | unknown(Sequence No. 3) | AK037760.1 |
| 6 | 355535 | cytotoxic T lymphocyte-associated | AY034578.1 |
| | | protein 2 beta | |
| 7 | 361748 | T lymphoma oncogene | NM_011601.1 |
| 8 | 368174 | RIKEN cDNA 2010323F13 gene | NM_177157.2 |
| 9 | 392437 | RIKEN cDNA 4930566F21 gene | AK016234.1 |
| 10 | 401554 | ribosomal protein S2 | AK004568.1 |
| 11 | 424366 | unknown(Sequence No. 4) | |
| 12 | 428440 | Otogelin | NM_013624.1 |
| 13 | 432053 | RIKEN cDNA 4933407A17 gene | AK016710.1 |
| 14 | 433675 | similar to T-cell receptor alpha chain V | M23096.1 |
| | | region PHDS58 precursor | |
| 15 | 460185 | predicted gene 3364 | AK090251.1 |
| 16 | 478590 | ring finger protein 149 | AK040602.1 |
| 17 | 497466 | cyclic nucleotide gated channel alpha 2 | NM_007724.2 |
| 18 | 513268 | RIKEN cDNA C030016D13 gene | NM_175354.2 |
| 19 | 515525 | unknown(Sequence No. 5) | AK053269.1 |
| 20 | 520252 | unknown(Sequence No. 6) | |
| 21 | 524988 | early growth response 1 | NM_007913.2 |
| 22 | 525785 | hypothetical protein 9130430E04 | NM_183203.1 |
| 23 | 530939 | RIKENcDNA9330176C04gene; | NM_175420.1 |
| | | solutecarrierfamily22 | |
| | | (organiccationtransporter), member13 | |
| 24 | 566320 | unknown(Sequence No. 7) | |
| 25 | 615742 | olfactory receptor 1350 | NM_146389.1 |
| 26 | 749804 | RIKEN cDNA 1200002N14 gene | NM_027878.1 |
| 27 | 762310 | RIKEN cDNA 4833417J20 gene | AK014716.1 |
| 28 | 781797 | olfactory receptor 704 | AY317825.1_{—} |
| | | | CDS_1 |
| 29 | 783471 | unknown(Sequence No. 8) | AK037520.1 |
| 30 | 824655 | unknown(Sequence No. 9) | AK037572.1 |
| 31 | 838496 | RIKEN cDNA A430057L12 gene | AK078093.1 |
| 32 | 841082 | RIKEN cDNA 9330198N18 gene | AK020395.1 |
| 33 | 847317 | myogenic differentiation 1 | NM_010866.1 |
| 34 | 850104 | gene model 44 | XM_907821 |
| 35 | 910205 | RIKEN cDNA F630021I08 gene | NM_178743.2 |
| 36 | 932668 | early growth response 2 | NM_010118.1 |
| 37 | 298812 | RIKEN cDNA 1700007H16 gene | NM_027945.1 |
| 38 | 334928 | matrix metalloproteinase 15 | NM_008609.2 |
| 39 | 338997 | Ras association (RalGDS/AF-6) domain | NM_175445.3 |
| | | family 2 | |
| 40 | 339413 | cDNA sequence BC020354 | NM_198110.1 |
| 41 | 357505 | high density lipoprotein (HDL) binding | NM_133808.2 |
| | | protein | |
| 42 | 357970 | cyclin-dependent kinase 19 | AK019224.1 |
| 43 | 370504 | potassiumvoltagegatedchannel, | NM_008420.3 |
| | | Shab-relatedsubfamily, member1 | |
| 44 | 391067 | kelch repeat and BTB (POZ) domain | NM_001164493.1 |
| | | containing 9 | |
| 45 | 396150 | transmembrane protein 120B | BC022593.1 |
| 46 | 398003 | Phosphofructokinase, muscle | NM_021514.2 |
| 47 | 444821 | unknown(Sequence No. 10) | |
| 48 | 463085 | breast carcinoma amplified sequence 3 | NM_138681.2 |
| 49 | 466313 | POU domain, class 3, transcription | NM_011141.1 |
| | | factor 1 | |
| 50 | 471089 | RIKEN cDNA B930093C12 gene | NM_172945.1 |
| 51 | 472358 | sterile alpha motif domain containing | BC049740.1 |
| | | 4 | |
| 52 | 475067 | Rho guanine nucleotide exchange factor | NM_027144.1 |
| | | (GEF) 12 | |
| 53 | 488058 | cDNA sequence BC052066 | NM_172761.1 |
| 54 | 491436 | fuzzy homolog (Drosophila) | BC045601.1 |
| 55 | 498825 | SEC5-like 1 (S. cerevisiae) | NM_025588.2 |
| 56 | 501289 | presenilin 1 | NM_008943.1 |
| 57 | 526436 | insulin-like growth factor binding | NM_010517.2 |
| | | protein 4 | |
| 58 | 531607 | unknown(Sequence No. 11) | |
| 59 | 540378 | RNA binding motif protein 8 | NM_025875.1 |
| 60 | 544433 | RIKEN cDNA A830097P10 gene | NM_173780.2 |
| 61 | 555234 | protein phosphatase 5, catalytic subunit | NM_011155.1 |
| 62 | 555859 | BMS1-like, ribosome assembly protein | NM_194339.1 |
| | | (yeast) | |
| 63 | 559958 | gene rich cluster, A gene | NM_013533.2 |
| 64 | 563270 | unknown(Sequence No. 12) | AK043558.1 |
| 65 | 563705 | RIKEN cDNA 2210009G21 gene | NM_028834.1 |
| 66 | 574782 | zinc finger, DHHC domain containing 14 | NM_146073.2 |
| 67 | 597728 | RIKEN cDNA 6430548M08 gene | NM_172286.2 |
| 68 | 602012 | dishevelled associated activator of | AY426536.1 |
| | | morphogenesis 2 | |
| 69 | 606287 | 3-monooxgenase/tryptophan 5-monooxgenase | NM_018871.2 |
| | | activation protein, gamma polypeptide | |
| 70 | 607746 | RIKEN cDNA 9330161C17 gene | NM_172614.2 |
| 71 | 638437 | histocompatibility2, Tregionlocus9; | NM_010399.2 |
| | | histocompatibility2, | |
| | | Tregionlocus22; histocompatibility2, | |
| | | Tregionlocus17; histocompatibility2, | |
| | | Tregionlocus10 | |
| 72 | 658280 | double C2, alpha | NM_010069.1 |
| 73 | 668016 | RIKEN cDNA D930014A20 gene | NM_178811.3 |
| 74 | 669220 | zinc finger, FYVE domain containing 20 | NM_030081.2 |
| 75 | 675690 | RIKEN cDNA 2310075C17 gene | AK010164.1 |
| 76 | 703524 | Synaptotagmin 12 | NM_134164.2 |
| 77 | 734020 | myosin XVIIIa | NM_011586.1 |
| 78 | 734899 | wingless-related MMTV integration site | NM_009524.2 |
| | | 5A | |
| 79 | 746773 | RIKEN cDNA 2610511O17 gene | NM_152817.2 |
| 80 | 757556 | XPA binding protein 2 | NM_026156.1 |
| 81 | 769898 | UDP-N-acetyl-alpha-D-galactosamine: | NM_139272.2 |
| | | polypeptideN- | |
| | | acetylgalactosaminyltransferase2 | |
| 82 | 829347 | RAD52 homolog B (S. cerevisiae) | NM_025654.1 |
| 83 | 849739 | peptidylprolyl isomerase (cyclophilin)- | NM_026845.1 |
| | | like 1 | |
| 84 | 858428 | toll interacting protein | NM_023764.2 |
| 85 | 894752 | ets variant gene 5 | NM_023794.2 |
| 86 | 896014 | eukaryotictranslationinitiationfactor2, | NM_012010.2 |
| | | subunit3, structuralgeneX-linked | |
| 87 | 898183 | glucose phosphate isomerase 1 | NM_008155 |
| 88 | 917144 | sodium channel, voltage-gated, type I, | NM_011322.2 |
| | | beta polypeptide | |
| 89 | 923175 | TRIO and F-actin binding protein | NM_138579.2 |
| 90 | 925691 | mitochondrial solute carrier protein | NM_030054.2 |

2. The diagnostic marker of claim 1, wherein the brain disease caused by brain injury is induced by MDMA (3,4-methylenedioxymethamphetamine hydrochloride).

3. The diagnostic marker of claim 1, wherein the marker predicts or diagnoses brain disease caused by brain injury which is induced by MDMA (3,4-methylenedioxymethamphetamine hydrochloride) in the next generation.

4. A diagnostic composition for brain disease caused by brain injury, the composition comprising a material for measuring the expression levels of one or more genes selected from 90 genes listed in the Table of claim 1 or the amount of protein expressed from the genes.

5. A method for predicting and diagnosing brain disease caused by brain injury, the method comprising the steps of:
(a) measuring the expression levels of one or more genes selected from 90 genes listed in the Table of claim 1 from a biological sample of a patient suspected of brain disease caused by brain injury or the amount of protein expressed therefrom; and
(b) measuring the expression levels of genes from samples of a normal control group or the amount of protein expressed therefrom and comparing the same with a measurement result of step (a).

6. The method of claim 5, wherein the biological sample is a cerebral cortical tissue or cell.

7. The method of claim 5, wherein the assay is selected from the group consisting of reverse transcriptase-polymerase chain reaction, real time-polymerase chain reaction, Western blotting, Northern blotting, ELISA (enzyme linked immunosorbent assay), RIA (radioimmunoassay), radioimmunodiffusion, and immunoprecipitation assay.

8. The method of claim 5, wherein, if the expression level of any one of the 1^{st} to 36^{th} genes among the 90 genes or the amount of protein expressed therefrom is under-expressed compared with samples of a normal control group, it is concluded that brain disease caused by brain injury was induced.

9. The method of claim 5, wherein, if the expression level of any one of the 37^{st} to 90^{th} genes among the 90 genes or the amount of protein expressed therefrom is over-expressed compared with samples of a normal control group, it is concluded that brain disease caused by brain injury was induced.

10. A method for screening a material for preventing or treating brain disease, the method comprising the steps of:
(a) contacting a sample to be analyzed with cerebral cortical cells of an individual with brain injury containing one or more genes selected from the group consisting of the 1^{st} to 36^{th} genes among the 90 marker genes listed in the table of claim 1 for diagnosing brain disease caused by brain injury according to the present invention or protein expressed therefrom;
(b) measuring the expression level of the selected gene or the amount or activity of protein expressed therefrom; and
(c) as a measurement result of step (b), if the expression level of the selected gene or the amount or activity of protein expressed therefrom is over-expressed, determining the sample as the material for preventing or treating brain disease caused by brain injury.

11. A method for screening a material for preventing or treating brain disease, the method comprising the steps of:
(a) contacting a sample to be analyzed with cerebral cortical cells of an individual with brain injury containing one or more genes selected from the group consisting of the 37^{st} to 90^{th} genes among the 90 marker genes listed in the table of claim 1 for diagnosing brain disease caused by brain injury according to the present invention or protein expressed therefrom;
(b) measuring the expression level of the selected gene or the amount or activity of protein expressed therefrom; and
(c) as a measurement result of step (b), if the expression level of the selected gene or the amount or activity of protein expressed therefrom is under-expressed, determining the sample as the material for preventing or treating brain disease caused by brain injury.

12. The diagnostic marker of claim 2, wherein the marker predicts or diagnoses brain disease caused by brain injury which is induced by MDMA (3,4-methylenedioxymethamphetamine hydrochloride) in the next generation.


SEQUENCE LISTING (96 sequences)

SEQ ID NO: 1 - DNA, 180 bp, Artificial Sequence
  probe ID 299424 gene sequence
gcatgctctcccagacttggtctaggcttgctggtgggtttgggggcctgatgacctactgtgcagaaatctcataagctccaaattggacctactatctgccaatagattaagcccttcaatccttttcctaagcaatctgcttgaggaccttctccagacatatatctaaaatgttcc

SEQ ID NO: 2 - DNA, 180 bp, Artificial Sequence
  Probe ID 305559 gene sequence
aagaggagacagctgagcagagccatccaggagcccgatgtcccaactcaccttccagccagtctcctggaatccaaccacatgagtggacctacgaggaacagttcaagcagctgtatgaactcgatgcggaccccaagaggaaggagtttctggatgacctatttagcttcatgcaga

SEQ ID NO: 3 - DNA, 180 bp, Artificial Sequence
  Probe ID 355052 gene sequence
gcaaccactgccctccgtttggagcttaaaaggaggctcccagtattctgaagtttaagtgatgccgcagagcataaccctgtgcctatgtattccacattactaaggtatatcttcagagtacatttctagttcttttgggtagagtgctgttgatgtctgtctccactccccaaattc

SEQ ID NO: 4 - DNA, 105 bp, Artificial Sequence
  Probe ID 424366 gene sequence
gtgccctggactgggcgaagtgggagtgctgggttctgatgatgatatatacaaaacctgatgcctgcaggtgggtcacaccatgcctctgtctgactacgaaga

SEQ ID NO: 5 - DNA, 180 bp, Artificial Sequence
  Probe ID 515525 gene sequence
ggctacacaataagatcttgtctcaaaacaacaagaacaagaatgacagttccatgattcataagagtcttttctgaggcttgctttatagtcatttcctctaacataaagcagaccatattggaaggaagctgcttaagctaagaacctaaacaactcaaaagctttaggaaattcctg

SEQ ID NO: 6 - DNA, 180 bp, Artificial Sequence
  Probe ID 520252 gene sequence
aaatccattctatgaacaactgaaattacagaacaagcaggtcatgtcctctgtgtacaaatcattgaagaagttaattgaggcccaagagaacatccacaagctcaatcaggagataagtctctattcaaacctacatagccggctcatggtggagaagaatcttactaagatgtccgt

SEQ ID NO: 7 - DNA, 180 bp, Artificial Sequence
  Probe ID 566320 gene sequence
aggaatccaaatgcgttagacccagccagagttctacacagccgccctcgcctttcaagccgctcaggtgacaaccggtttgctccatgaaaactacctgacttcagcttgctgtctataccctgaagatccccagagtaacagcttgtccttcctcaaggctctttgcttaggaagaaa

SEQ ID NO: 8 - DNA, 180 bp, Artificial Sequence
  Probe ID 783471 gene sequence
taagcattggttacaatgaaacttaaatgtgcctaatactaagatctttccatctctgactcatggggtaatgcaaaattgtttttaaaatagtaacttttcatttgaaaggtactttatatcaggctcaaatttagtacctatttatatgctgagcaataggggtgggcacatacatca

SEQ ID NO: 9 - DNA, 180 bp, Artificial Sequence
  Probe ID 824655 gene sequence
ccccatgtcttcaaatcctgcctctttaaacatgtgaaccattatcactagggtacactcaagtgctagccactccatactacatccctgttctggagatacccacaagtttttacagatcgggaaactgaggctctgagaggtggggtgagaggtatagagcttatggcacagtcagga

SEQ ID NO: 10 - DNA, 180 bp, Artificial Sequence
  Probe ID 444821 gene sequence
gaagtctactggcatcttcaccaccatggagaaggctggggcccacttcatgggtggggccaagaggatcatcatccccccaccttctgttgatgcccccatgtttgtgatgggtgtgaaccacaagaaatatgtcagcaatgcatcctgcaccaccaactgcttagctcccctggccaa

SEQ ID NO: 11 - DNA, 180 bp, Artificial Sequence
  Probe ID 531607 gene sequence
attgttgccatcaacaaccccttcattgacctcaactacatggtctacgtgttccagtatgactccacccatggcaaatccaatggcacagtcaaggttgagaatgggaaacttgtcatcaatgggaagcccatcaccgtcttccaggagcgagatcccgctaacatcaaatggggtgat

SEQ ID NO: 12 - DNA, 108 bp, Artificial Sequence
  Probe ID 563270 gene sequence
ttctaatctctcatttatgtgctgttgtggcaagagggctaacagtttgagaaaccactgtaagcacaggtggactttctaaataggagaaaaaaatttgcagactgt

SEQ ID NO: 13 - DNA, 2796 bp, Homo sapiens
gagaaggccagcaacgttcctgactgcacgcaggagtctattagtctattagtctacagcttgggcaaatggaagacttgtcccagcctcagtttctccatccacaaacagggacgatgataatgatacgcacccagagagctggatgaggatcaaatgggctttttggcacttagaggcgctgtgcagcaagcaggcatagctggcttacatcttcaccaccaaccgccctctcccaaagacccactccagaaacctgcccccgaccgaggcgcccatctcacctggcttcgtacacaggtaggagtaaaagccctcggacttgagcatgatgagcagcgaaccccagcccaggaggactgcggagaaaaggaggttttccaacacagcggtgcaggccatccaccagcggcgccgatgggcagtggccagggtgggcgccatggtgcggtgtggcggtggtcgggctccggttctgcagacacctgcgcgcagacctcagcttcagcggtggctctacccaacccgtgtagccgctggcgcacagcctgtcagtcccttgcagcccaggctataggtgcccgccgcaaaagaaaagaaccctagccctgtcagggacctgggccaccccgtacccgcagctacctggtaacgctaccttaggccacagtggagccccgctggccaagaggaaaggcttctcccttagttcactcggaggtaaactgaggcatggcagctggaaaagggctaattatgctcaaggacgtggagcgggtcctgatcccaagcaagactgaacagccactttgggggccggaaagggaggcagctcgtagtaaggtcacctgagcagaggctcacctgagagctgccactcccagggaccctgacaggctcccgcccgcccgcactcgagacccttccttagtgcttgttccttcaaccacaaaatcagcacagggagccccgaaacggcccgcccacggcgctttcattggcgggaccgcacaggaactgccgcgccattggttagtgtttaggtcgctaggcgctaccgccacgcgaaccccgcatgggagaggcgaagccggttcaaaccggcggcgcgagccaaccgcgcgacccccgtgtcgggctgttaccccggcgattagcccgtttccatagaagcggatcccgtccctctgtcctcctggcctttttcgttacgtctccctcccggtgtcgtccctctgacaagtatgcaccttccctccaacccctgcctgcgagcggcttcaggttctcgcaccctcgtctgagacccggatcctgggagtccagctcacccggctcctaccagtgtgtatcgggcccgcccggcgccttttctgtcccctttatttccagcccttttattcctatcctaagatgaaaaacactgccgcccgttgattggctgagtggggctggaaacaggcgtcggccaatcagcagctacccacccgccccccggcgagcagaagccggttccggccggacttggagacaaacaagaggaatgggggggctggggggcggctcgggcaggcccagctctacccagttaaactggagtgcccctcgacccggtgaccgccgcgggcatcgccccacgcccttctcgttgccctcttccttgtcaagttcatgggtcagcgtttcagggagtcttggtccacagcgagctgggctccaagcaccgtgcggttcggcccgcagtatagaccaccgttctcgcccggagagaaccagggccggatgctccaactccgcaggccattaccgcccttttgtgctgtttcccggcctgctccttgggctttgtgtccgggctcctcgctgcctagcccccgttgtcctatccaggcgctgtctacgggtatcagaacaccccgacccaggtcgtggcataattaccgcatccctgctagatctcccgactcaccgggtccttgcctggaatcaggtgcctcctgcctggtctttaatcggtgtcccttaagggtctgtagtcaggattttggactaagagcccctggtcctccagttgtcgccatccccatcccgcgccctcttttctcggtgccttcagagaccggtcagtgttcaggccggtcaccttggcatgccctgccctcccactttcccaaacgactctttcatgtggctggcacactttctcgtccacccttcttcatgctgtgagcaatcccgagcacctagttagtctaagtctgactttggagagcgcgatagatcactaggggtccaaagagctccgtcctttctgtatcatcagcggagctgcgctgctctgaggacctgcttggacgtggaatggagcctcagcacgaaagatttggctgtccgtccgagtctccctgacgagaggcactaacctcccctcagaaaacactcggtatccctcagattctccacacttcaagagccggcttgctttagagctaagggtgtccccagcagcctgactcagcttccctcagctcgagtgcgccccctggtgcccagaaccttcgccagccattctgctagtgcatgctctcccagacttggtctaggcttgctggtgggtttgggggcctgatgacctactgtgcagaaatctcataagctccaaattggacctactatctgccaatagattaagcccttcaatccttttcctaagcaatctgcttgaggaccttctccagacatatatctaaaatgttccctccccctcttcaaataaatagtgcccccttctccctc

SEQ ID NO: 14 - DNA, 639 bp, Homo sapiens
atggctcccttgaccgaagtcggggctttcttgggcggcctggagggcttgggccagcaggtggggtcgcacttcttgctgcctcctgcaggggagaggccaccgctgttaggggagcggcggggcgcgttggagaggggcgcccgcggcgggcccggttccgtggagctggcgcacctgcacggcatcctgcgccgccggcagctctactgccgcaccggcttccacctgcagatcctgcccgacggcaccgtgcagggcacccggcaggatcacagtctcttcggtatcctggaattcatcagtgtggcagtgggactggtcagtatcagaggtgtggacagtggcctgtaccttgggatgaatgacaaaggagaactttatggatcagagaaattgacttctgaatgcatcttcagggaacaatttgaagagaactggtataatacctattcgtccaacatatataaacatggaaacacgggtcgcaggtattttgtagcacttaacaaggatggaactccaagagatggtgccaggtccaaaagacgtcaaaagtttacccactttttaccaagaccagtagacccagaaagagttccagaattatacaaagacctactgatgtacactggatga

SEQ ID NO: 15 - DNA, 637 bp, Homo sapiens
gcctttgggacgggaatagcgtgaggacaagatgaggcccagagtctcctggtttcagtggcagatctgtcatgattcttcaccggaggatttagatttaataaggcttacaagattaggattttagttgctgcacccagtgattgagttaccgttgtttctgaactaggtttgtgttgtgttttccttcacgtggtgactcatctgggtccaagagaggaaggtatggcggcaaagcgtgggtttgcctgatgtgcaccacaaagaccgtgggggatttgaagcacggggttggcatggtagcgacccgccagtaggaatttaataagctgggtgaaaacattgtcgagttatgaatcaaagtctccacaagttaaagacaggtcggccattcccgccagtgtctggccggccaggccactgggacagaggcacaagtgaaggacatgacggttcttttttttttaatatttcctgcaacatgtggcaatatatgaagtgccagagggcacagctcgcatgaaggcaggtcctcatctctgggactcgtttgttgtgttttaaagttttttagaaaagttataaagaaaatccatctacatgtatcattttgataaaaataaaaaatttaatgttc

SEQ ID NO: 16 - DNA, 2479 bp, Homo sapiens
gaggaaataacatatataattttaaattttaaccatgatgtagaacacaagtgtagtcatgaacgtatttgctgccttgtatgtttgttgtaggagacaggtgtttgagaagtggcatacggtcttcaagaggaatgagtcaaaggatagattgtggataccttggaggccacaggtttgttaccgctttgctgagttatatgggtttgtcttctcaagggggtgaggaacagaaaacatcctttaccatctgaacactatgaaaccgattagatgaacagacactggagagaatgtcagatactattctgaacaatggttgctggtgccttcagtctgtacagcaagagcagaccatagaaacaaactgattttcttgaagttaaaggagtgtgttagtctcctggtgctgctataagagaccaccacagcctgggttattcctcacactggcagctctgttggatgaaggccccacccttttacctcacctaaaacttcatcaccagaaaagtcatattggaatataaggcttgtgcataggagttttagaggagaaatagctaagtctataactgagaaattcaggaagtccacactgtcctggaaggctcggcaaggcctgcagagttcctgatttttgtctcatgtctatcaagctccaaggtggtgctgatgctgttgctctgggaactgtacttggaggattgcttttagattgcgggggtgggatgggggtgtcacccttgagaggtgtgagacaggggtatggcagaaacagaaggcagtgggacgttctggagactttcttggttcagcttaccaagttatgtttagtcccgatttgagacattcaaagaggtttgattgaagctccatcactgacattgtctgggaaataagtgctaattttattaacagtcttactccctggaatattcagaattttggtcaacttttgtccccctgcctttgccacttccgatcttagtctcctgtcccagtctgatgcgtgcttctgaggctctggctcctctcagtgcatacgtacacaggggattcccgatcctctcctcttctgttctgtcatgtgcttctctttacaagtagcctttcttctgctcgctctctctctctctctctctctctctctctctctctctctctctctccctccctccctccctccctccccagaataaacagacttgcatagctgtcctctttcttgtattcttcacaaaaggctgacacgttcccttgtacttggtatattttagaaactccaccatattggctggtagacagcagcttccctgtcacaggtgtgttgtgttctgatctgtgggccattctgtttgcaaccactgccctccgtttggagcttaaaaggaggctcccagtattctgaagtttaagtgatgccgcagagcataaccctgtgcctatgtattccacattactaaggtatatcttcagagtacatttctagttcttttgggtagagtgctgttgatgtctgtctccactccccaaattcctgccatgctcttgtctgtggactgggttcatgatgtaaggatgccttattaaaataatgaagtttagaaacaatagataatggtggtagtgaggagaactccaggggaatctgtgtccttggttcgtttctttggtttttacagcagccttaatttccttcctggtcgcgtctgctgcttctccagaagcactgtttatgtgagtgtgccatttgccttgctgttgccttgcaaccttctgtgtaattgggtgttaccatgaccatccgtatttgaatattcatccttttggcacttggtatttgattttttttttcccctctgtgagtgacttcatctcgtttgtctgtgtcatttggtctcctccttttcacaagatttcttaagtctccttttactcccgtgataggtgccatagactcgggtgggctgtgattagtgcccacagactcatggacggtggaggccctgttggtttgtggtgtttattatgcacagtcattcgaagtctctccttagaatattggaagccatgtgtggctttgtttgtacttcctgtggttttaagctttgagaaaggagagtttgggatttagggttttgcagttatcctcctggctcagaagttgtaaaggaacctttcataatcaaggttttcgtatgctaatctaggtcacatactctcctcctgtagggctctgtgtctaagtctttgcattacatatacatgtacatgtacatatacaagcatgcagatgtagatgttcttacaatgacctagtgcagacctagtttgccaagctgtttaccgtaggccacggataagcagactcttctttaaaaggtcattttttaaatttttgaagttacagaaaccaaataatctttgtgaaattcttgctcctccctatccttttcccctcttctttcccctcccc

SEQ ID NO: 17 - DNA, 777 bp, Homo sapiens
ctggacaacaaagttctggtttctatctgtgaacagaagctgcagcacttcagtgctgtcttcctgctcatcctctgcttgggaatgatgtcagctgctccatcccctgatccaagtttggataatgagtggaaagaatggaagacaacatttgcaaaagcctacagtctggatgaagaaagacacagaagactcatgtgggaggagaataagaagaaaattgaggcacacaatgcagactatgagcggggcaagaccagcttctacatgggcctgaatcaatttagtgacttgactccagaagaattcaggacaaattgctgtggaagctcaatgtgtagaggagaaatggctcctgatttgcctgaatatgaagatttgggaaagaacagctatctgacacctggaagggctcagccagagtaacagttgtggcttgactggtaacaatatcatagtctaggagtgaagcaagcactctgcattgtcttgggagtcttctgtctcctggtatgggaggaatgatgctgtagaggctcaacagcaggaagcagcccatcacagtgtccagcatgtgctgtgtgatatgcaaaactgaactcatataagatcaacactgtggtgtgaactctgaggcacagtcatactgagaaatcattgccaatttttagctctgaagttccttaaatgattctcacaaggatctttagttttcactttaataagatgaatatatgccatgatgtcttcaaattaaagctagatattaaattaaaa

SEQ ID NO: 18 - DNA, 954 bp, Homo sapiens
atgagaacccatctccttctctccagacctcagtcatctagaggacctgcggctgggcacagagcaagacacactgaccttctggtgcttttagagtccccagcccctgttctctccactgttatgatgtgtctcctctggtggccagctcctgccaagatcacttcacactctctcatccaattaacaaatcaatgcgtttgcctttactttgctccagggcatatgtgtgtgtgggtgctcccaggccaagcctgcagtatggcactacagagcttcttccttgtgtctctctggctacttccctggcaaaacccagcacggtttttccttccttctctaatcaacagaggattctttgattcaatgtcacccctccaatgcactcttgttctgtctgtggtcctagcctctcttgttcaatggttggctgtgagcatccatctctgtatttgtaaggctctgtcagggcctctcaggagacagccatatcaggctcctttcagcatgtacttcttggtatccacaatagtgtctgggtttggtaactgtatatgggatgaatctccaggtggcacagtgagtggtctttcctttagtatctcttctacacttttatctccatatttgctcatgtattttgttcttcttctaagaaggcacggtagtactatgcccaattttctgaggaactgtcaaactgatttccagagtggttgtactagcttgcaatctcaccagcaatggaggagtgtccttctttctccacatcctcgtcagcatctgttgccatctgaatttttgattttagccattttgactggtgtgagatggaatctcagggttgttttgatttgcatttccctgatgactaaggatgtctttaactgcttctcagccattcgagattcctcagttgataatcctttgtttagctctgtaccccatttttaa

SEQ ID NO: 19 - DNA, 631 bp, Homo sapiens
gagagcagggagtaacgggactgtggtcgcgggcagccccagccacgcaccatgccctatctgctcatcagcactcagatccgtatggaggtgggccccaccatggtgggtgatgaacactcggatccggagctgatgcagcacctgggggcctccaagagaagtgtcttgggaaacaacttttacgaatactacgtcaacgaccctcctcgcatagtcctggacaagctggaatgcaagggcttccgagtgctgagcatgacaggggtgggccagacgctggtgtggtgtctgcacaaggaatgagctgcttgagctgacctgttcagagaccgccatgtgccctgcccacatccttgcatcactgtttgccctgcctatctctttccaaattgtcaactcccttgcccaatactggtgggctgtgaacagcctaagggacctcgctccagctggttgggcacagcaggctcctgccctggtgctttcagcttcccccagctaggcctgaccctggctcttgctgtaggccacatgctgaaggcaagccaacccgaggacgaggacagtccctggatcctacacagctaatccaagccaataaagggctctcacaaagtggttgttcctc

SEQ ID NO: 20 - DNA, 1535 bp, Homo sapiens
gacaactctccacgccaaccagagtagtagacgttacactcccctactctttaggcccgggccttcggaaagctgccaattcgcagcaagacaagttcagctgtaccaagtgtgcattaaggttgcccaggagccggcctcaagtgacccaggctagggttcgaggcttcgggaaggaaaggagtcagaggacagctcgagaaaaccacgcgcccggacccgcagattccccagacaacttcctcccttcttcccgggcctgaggcgccactaccgcttctgcacggtgcacgtcacctgtacaaaggtgtgtctttcgctgcaagaactgcaagtgtgcctggtggtccttccgagccgtctcagctttcacatcctccagttccttctctaacatctggattttcatctcctggctcttctgtatggaggaaatgtgctccattgactgaaactgctgctccaactgggactgcttgatttttccctggaagagttgtgtttgaagctcctcagtttgcttggcgaacctgagtgtcagttcttgtttcttcgaataatctccccaaggtcctgaacatactgatctcacagttgcgcgtgtttctttttacactgctcactctgcttccttaagactctcaaaaagcagttcctctcagcgtccacacagagagtgtcacgctgcagcagcctgctgtcttgcagcaggtgctcctgctggagcctggcctcttctatttttctgctcaggctctcaagtgcctccattgtcttgctttttacctccttctctaattctgtcagcctctctgacttgaaaagttcatttagaatagtaagatacgttgatggctgaggagaaccttttgaactctgggcaaacctggaggaagaacaaaggaaatattcttttatcagttctctaggcacaagtgagaaaataatatctggaaactaaacctttgggttccatcatgcccaagaagggtcagtgcgggttagatttttgacaagttggtacattcaggaactatctgggaggaggaactgaatcgagaaaagggactccatcagattatctgtagaacagacagacactctctctctcaaagatttatttatttttatatcttaagtacactgtagctgtcttcagacacaccagaagagggcatcagatctcattacggttggttgtgaggcaccatgtggttgctgggatttgaactcaggacctccggaagagcagtcagtgctcttaactgctgagccatctctccagcccctctgtctctcttcaaattaaagatagattgatgtgggtaggcccagcccttgagggtaaggtgacacccctaggcaggtggtccttaactgaatacgaaagcagtccaagcaactgaacaatttatctctgtggcttctgcttcagttcctgcctccaggttcctgtcttgagctcctggccctgacttctttggatgatgaactgtgatacagaagaaaaagatgaaataaaccctttctcaatg

SEQ ID NO: 21 - DNA, 1012 bp, Homo sapiens
gcttcttcttcttcgggagaacaccaaatggcggatgacgccggtgcagcgggagggcccggaggacccgggggcccaggattaggtggccgcggcggcttccgcggaggattcggcagcggtcttaggggccgtggtcgtggccgaggccgtggccgtggtcgaggccgcggggctcgtggaggtaaagctgaagacaaggagtggatccccgtcaccaagctgggccgtctggttaaggacatgaagatcaagtccttggaggagatctacctgttctccctgcccattaaggagtctgagatcattgatttcttcctgggtgcgtccctaaaggatgaggttctgaaaatcatgccagtgcagaagcagactcgggctggccagcggaccaggttcaaggtgacaggccgctgtggctctgtgctggtgcgtctcatccctgcccccagaggcactggcattgtctctgctcctgtgcccaagaagctcctgatgatggccggtatagatgactgctacacttcagccagaggctgcactgccaccctgggcaactttgctaaggccacctttgatgccatctccaagacttacagctacctgactcccgacctctggaaagagactgtcttcaccaagtctccttatcaggaattcacggatcatcttgtgaaaacccacaccagagtctctgttcagaggagccaggctccagctgtggctaccacataagggtttttatatgagaaagataaaagaattaagtctgctgaaaaaaaaaaaaaaaaaaaaaacaaaaaagaaacaacaaaaagagaaaaaagaaaaaaagagacaacccgaaaaatagaagacgagtgtggggcggggggggggcgggggggcgcgccgctcggcgacgtattataaattcatacgcatgtttgtataaatagagagagggtatatacgggcgccaccgccctctggcagcacatcctctcccagcgggcggacgagacggtgcgcg

SEQ ID NO: 22 - DNA, 10052 bp, Homo sapiens
agcagagccctgggccaggctctctgattgcctggcaccctgatgggagcccgcatgcccagacgatgcttgcttcttctctcttgcttttgtctacttagggttgagtccactgcagaggtgcagcaccaggcctcagcactcacatggaaaatcagtgcagagctgcaacaggagcctgctcctgagcccagccacacataccaggagatgtcccttgcagtggaggatgtcacaacagttatggaaggccaggaggctgaagccctagccgcatcggccatgtcttcgtgggaaagacggctccatcgcgccaagtgtgcaccgtcgtacttgttctcctgcttcaatgggggtgagtgtgtgcacccagctctctgtgactgcaggcgcttcaatgccactggaccccgttgccagttggtgtataatgtaggtcctgagcgggacagcatctgccggacctgggggcagcaccacgtggagacgtttgacggcctgtactactacttttctggaaagggcagctacacgctggtgggacaccacgaacccgagggtcagagtttttccatccaggtgcacaatgacccacagtgtggctctgcccactacacctgcccgaggtctgtcagcctcttcctctcaggtgaacgtgagatctgcctggccaaggaagtcacccatggaggtgtaagggtccagctgccgcaggtggtggggggtgtgcagctgcagcagctggctggttacgtcatcgcgaggcatccgtcagccttcaccctggcctgggatggtatctcagccatttacatcaagatgagccctgagtttctgggctggacccatgggctgtgtgggaacaacaatgctgaccctcaggatgacctggtgaccagttacgggaaggtgactgatgatgtaggggagtttgtacacagctggcaggagcaggtccccaacagccctccaggacctgtgactacttccctgcctcgcccaccatgcctgcagcagagcccagcatccatgcagggtgtgtatgagcggtgtgaagtactgttgaggcctcctttcgacacctgccatgcttatgtcagccctctgcccttcgcagccagctgtaccagtgacctctgccaatcaggaggcgatgaagccacttggtgccgagcactgatggaatatgcccgggcatgtgcacaggcagggcgccctctgcagggctggaggacccaactctcacaatgcactgtgcactgcaaagagaaggccttcatctataacgagtgtattgcttgctgtccggcttcctgccaatcccgtgcttcatgtgtagacagtgagatcacctgtgtggatggctgctactgccccaatgggctgatctttgaggatggaggctgcatgtcaccagctgagtgtccctgtgaattccatggaaccctgcacccgcctggctctgtggtgacagaagactgcaatgcctgtacgtgcacagcagggaagtgggtgtgcagctcttccgtctgtccagctgagtgctcagtgactggtgacattcacttcacgacctttgatggacgccggtacacattccctgccacgtgtcagtacatcctcgccaagagccgttcctcaggcacatttacagtgacgctgcagaacgccccgtgtggcccgaaccaggatggggcctgtgtccagtccgtgtcagtgatcctgcaccaggaccctcggagacaggtgaccctgacccaggcaggagatgtccttctatttgaccagtacaagatcaccccaccctactcagatgatgcttttgagatccggaggctgtcctccgtgttcctgagggtgaggaccaatgtgggcgtgcggatcctctatgaccgggaagggttgcggctttacctgcaagtggaccagcgatgggtggaggacaccgtgggcctctgtggcaccttcaacggcaacacacaggacgacttcctgtctccagtgggtgtacctgagagcacaccacagttgtttggcaattcgtggaaaaccctgtctgcctgtgtcccgctggtccctggctctttgctggacccctgcgatgtgcatctgcaagctgcctcctatgcactgcagtcctgcagtgtgctcacgggagagctctttgcaccctgctctgcgtatctgagcccaataccctactttgagcaatgccgcagggatgcctgccgctgtgggcagccctgcctttgtgccacgctggcccactacgcacgactatgccggcgccatggactcccggttgacttccgtgcccatctgccagcctgtgcactgtcctgtgaggccactaaagagtacagcccttgtgtgaccctgtgcgcaccaacatgccaggacctggccagccctgacgtctgtggggctaacggaggtggcaacttcagcagggaggagtgcgtagagggttgcacctgcccaccggacacatttctggacacacaggctgacctctgtgtccccaggaaccaatgctcttgccatttccaaggagtggactacccccctggggacagtgacatcccttccctgggccactgccactgcaaagatggggtcatgagctgtgacagccgagccccagctgctgcatgtccagcaggccaggtgttcgtgaactgcagtgagctgcacccagaccctgagctcagcagggagaggacgtgtgagcagcagctgctgaacctgagcatgccagcacgtggcccctgcctgtcgggctgtgcctgtccccagggcctgctcagacatggggacgcatgcttcccaccagaggaatgtccatgtacctggaaggggaaggaatatttccctggggaccaggtagtctcttcctgccacacctgtgtgtgccagcggggctcgttccagtgcaccctgcacccctgcgcctccacctgtaccgcctatggggaccgacactaccgcacatttgacgggcttccgtatgactttgtaggggcgtgcaaagtgcacctggtcaagagtacatccgagctcagcttctctgtcatggtggaggatgtgaactgctatggctccggcgtcatctgcaggaagtccatctccatcaacgttggcagctcactcatcatctttgatgacgactccggggatcccagtcccgagagcttcctggatgagaagcaggctgtccacatctggagagcaggctttttcacactggtgcatttcccacgggaacacatcaccctcttgtgggaccagagaaccactgtgcatgtccaggcgggccctcagtggcagggtcagttggtgggcctctgtgggaactttgacctgaaaaccatcaatgagatgcggaccccagagaacctggagttaacaaacccccaggagtttggaagcagttgggctgcggtggagtgcccagatacccctgatcccggggacacttgtgtcctgaatcctctccgagaaccatttgcaaggaaggagtgtggtattctgctcagtgaggtgttcgagacctgccacccagtggttgatgtcacttggttttactcaaactgcctgacggacacatgtggctgcagccggggtggcgactgtgagtgcttctgtgccagtgtagctgcctatgcccatcagtgttgtcagcacggggtggtcattgactggcggacaccccgcatctgcccatatgactgtgacttcttcaacaaagcgctaggtaagggtccctaccagctgtccagtgtggcggctggcggtactcttgtggccacgaaggcagtagacagtgacatagcccttgtgagggcagaggatctggcgccaggggacatttcaagcttcctgctgacagctgctctgtacaaggccaaggcccatgaccctgacgtggtgtccctggaggcggctgacagacccaacttcttcctgcacaccacagccaatggctctatagggctggctaagtggcagcgcgatgaggccttccatcagcacgcttccttctcgctgcaccgggggacatggcaggctggcctggtggctctggagtccctggcaaagcctggctccttcctccactcatcaggccttgagctggcactgcgggcatacgaacacacggaggtgtttcgtggtggtgcgctcttccgccttctggatgccaagcctttgggggctgcctaccccacctgtgagtggcgctacgatgcctgtgctagcccctgtttccaaacctgccgggacccacgggcaaccagctgccaggatgtgccaggtagaagctgtgtccctgtgtgtcccactcctaaggtcctggatgaagtcacacagagatgtgtctacctggaagactgtgtggagccagccccgcggggtcccacggagacccttggcaatgagactctggtacctggtcaagtgccacccaccaccagtgccgagcagcagcttccacaggggctccctggagcctctgcctacagccccgcccctgttccagtagccccacccacctcagccccaaacccaccaatggctgccacggaggggcaagcgccatctccaggatccacccaacccacactgcagacacccctgggccttacaacatctaactttcctgctggccacacagaggccacagccagagaggaaggggctgccagccttctaaccacttctcatcccccagggttctcgtcctccctccctagttccctgcagatgcccacatctggcattgtatcaggtgcaacagagacaaccaaagtgaccataacctttacagggagtcccaacaccaccgtcgcttcccggtcaccccctatacctcgcttcccactcatgaccaaagctgtgacagtcccaagtcatgactcctttcctgttaaaaccacacctctgcagccctcttggctatggagcctctcttccagacctatgacttcccttggagctacgtcctggccccccacctccccgggatcccacctgtcaactgcagtaactaaggtggcaaataagacaatgacatccctgtctgtcctggcccagagcacttcaagttccagccagcccctggctgcagtgaccacagcacacagggctcctgccagtccccttgtcaccaagggcttggaggtggtgtcagccacagagaagggagaagctgggcacagccagcttacagagttgccagtgtccccacccccaagcccagcccccatcgacctgcctcacccagcccaacataccaccacagcccctgggccttctgctctgtccccggggatcctggctgctggcagtccatccacaggtgctcacagaccgggggccacagctttagcttctctagagccaactcggccaccccaccttctctctggcctacctctggacaccagcctgcccttggctaaggtgggaacctctgccccagtggccacacctggctccaaaggctatatccccacccctccacctcagcaccaggctacaactttggctacggccatgactgtgagcccacttactcagtcactcagtctgacagtgccactcatgtctgctgtagaggaacaggcacactcacctagccccaagcctccccaaggaacaggtatggctccagaccagatgctgggagccacattgccaagctttggagcctcctctgtgattgctggggtgccccccacagtgtctgcggccccacggaagagcaccacccagagagcagccattctatctaagaaggtgtctccacccaccctcatctctgactctgtccagggtgggttcacagaactcacacctatagtgagccatactgttacccccttggcgactgaggctgaaggcccccgggctggcacagtgccactggtgcccacaacctactccctaagccgggtgtcagccaggacggcctcccgggaaggcccgctagttctgttgcctcagctggcagaggcctatgggaccccagcaggactccagccccaagaagatctaatgcgccaggccactacggagcagtcagggcgctcagctccagcccagagtatagcagaggagtcgatggaggccgaagtgaatacttctgccacctgtgtgcccatcgctgaacaagactgtgtccatcacatatgcctagaaggccagctgatacgtgtgaaccagacccagcactgtcctcagggtgccgtacgccctcgctgtggggtcctgggcctcgctgtgcgggtgggcggggatcgctgctgcccacaatgggaatgtgcctgccgatgctcaatcttcccagacctgagctttgtaactttcgatggcagccatgcggctctgttcaaggaggccatctatgtcctcagccagagcccagatgaaaccatctctgtgcatgtcctggactgcaagagtgccaacctgggacatctgaactggcccccattctgtctggtgatactgaatgtgactcatctggcccaccatgtcagcatcgaccgcttcaacaggaaggtgactgtggactctcaagttgtgtggccacccatgagccgatatggcttccgaattgaggatacaggacacatgtatatggtccggacaccctcacacatccagatccagtggcttcacagctcaggactcatgatcttggaagccagcaaagtcagcaagacccagggccatggcctgtgtggtatctgtgatggagatgcagccaatgacctcaccctgaaggatggctctgtgctgggtgaggcagaggaccctgctccctttctggacagctggcaggtgcccagctccctgacttcagagggtcagacccgcttccgcccagacagttgtgccacagctgactgttcgccctgccttcgcatggtgtccaaccgaaccttcagtgcctgccacagctttgtctccccagagtcattctgtgagctgtggatccgggacaccaagtacgtgcagcagccctgtgtggcactcaccgtgtatgtggctatgtgtcacaaattccacgtgtgcatcgagtggcggggctccgactattgccccttcctgtgctccagtgactctacctaccaggcctgtgtggcagcttgtgaaccccctgacacatgccaggacggggtactgggccccctggacccagagcagtgccaggtgctgggtgagggctgtgtgtgcacggagggcaccattctgcaccgacgccactcggcactctgcatccctgaggacaagtgtgcctgcactgacagcaccggggttccacgtgctctgggggagacctggaacagctccctcagtggctgctgtcagcagcaatgccaggctccagacaccatcatacccgtggacctggactgccctggtccccgacctgagagctgcccccgctttggtgaagtgatcctgttacagcccacggaggacccctgctgcctggggagtgtctgcgtgtgtaaccagaccctgtgcgagggcctcgctcccacctgccgcccaggccacagcctcatcacccacttccaggaggattcctgctgtcccagctacagctgtgagtgtgaccccggtctgtgtgaggctgagcaggtccccacctgtcgtgaggaccagatcttgatcgagggccgcctaggagactcctgctgcacctcctacttctgtggctgtggtgagtgttcagatcccatgcctgagtgtcaggaaggagaggcactcactgtgcacaggaacaccacagagctctgctgccctctgtatcagtgtgtgtgtgagaacttccgctgcccacaggtacagtgtggcatgggcacttccctggtggaggtgtggagcccagaccgatgctgcccctacaagtcctgcgagtgtgactgtgacactattcctgtgcccagatgccatctgtgggaaaagtcgcagctggatgaggagtttatgcacagcgtggagaatgtgtgcggctgcgccaagtacgagtgcgtgaaagcccctgtatgcctgagccgtgagctgggtgtgatgcagccaggccagactgtggtagagctctcagcagatggtgtgtgtcatacgtcccgatgcacagacgtgctggaccctctcaccaacttctaccagatcaacatcacctctgtcctctgtgacatgcactgcgaggcgaaccaggagtatgaccacccacgggacctggcggcctgctgcggctcctgcagaaacgtgtcctgcctcttcaccttccccaatggcaccacttccctgttcttgcctggtgcatcctggatcgcggactgtgcccgccatcactgtggcagcacccccctgggtgctgtgcttgtccgctcgcccatcagctgccctccattcaatgaaaccgagtgtgccaaggttggtggctctgtggtaccatccttggaaggatgctgcagagcatgtaaggaggatggacggtcttgcaagaaggtgaccattcgcatgaccatccgcaagaatgactgcaggagcaatactcctgtgaacctggtgtcttgcgatgggaggtgcccttctgccagcatctacaaccacaacatcaacacctatgcccgcttctgcaagtgctgccgtgaggtgggactgcagcgtcgctctgtgcagctcttctgtgccacgaatgctacctgggtgccctatacagtacaggagcctacagactgtgcctgccagtggtcctgaagcctgggtgccaggcctggctgggtcacccttgtcactttcatgttctccttccagctagtctaggtgacaaaggggagaggttaaaggtgggggatcttgtttgtatgtgcacgtgtgtgtgtgtgtgtgtgtgtgtgtgtgtgtgtgtgtgtgtaatccacaaggactagaataggccccacttgcccatcctggctctctctctctgagtccttttacatgcacatagaaagtgccattgctcagggcttttgggatccaactctacatggcttgaggtcaggagggaagctgtaggcccaggaactgaaggcctgatccatctgtgtctctggccaggacttctagggggaggggccaaggtttcctgatacactgcaaccaaagcagaacccaatgctggcttggggttaagatttggtggcctgcttctttcttgtccctcttaacaatggaaaacccataatataaggaaaggggatttaactaacatttctgactgtctcaagaggtctagaactccttgaagtgaagagggtgagcttggggctcagggatgcccatgaagatctgtgagaccatgctgtgactgagcaggtggctctagactcctcaagcctcagagcgttccctgacattctggtatctagaagagcaggtccaggtctgtgcctctgtgtgtcacatgctcaccattgagccaggtttgagatacatgaaggataggcttctcagtgagggcctgggtgccaatcccagagggtgctggacagcccaaccctccaagcagttctggagtacacagctggcacttccagcctaaagccctgtcccttttctccaagcatgtgaaaagtgtatctgcactaggtcttactgtgttaggtggtggctcagcgctgcctgggactgtgttggtggagaatgtctgtaggtgaaaatgcagccctgtgagtttgcaacccaggtggccccgagtgtccgtcagagccttgtctccaaaccaaagtgtcttccctaaagccaggatcagagctcctgtccacaagccactttgcagatcccctcctttctgcactgctttgtgctactgggtctcaaaggtcatgacctgctgctgttcaggtcccagcactgttctgacggccccatggggtaaaggtcctgtgtcgagatggtcccaacatccgctttgtctctgtgactcttctgtatcttcttcatgtatcaataaactgcaagaagttttccaagcaaaaaaaaaaaaaaaaaaa

SEQ ID NO: 23 - DNA, 2144 bp, Homo sapiens
aggtgtcagattgttcatttgaattgttacttattaatgtttgtttgtattctattttcaaacttcctattaatccactgagtctttttttttaaacatttaaggtttttatcatgaatattttgtttcttctctaattataatttttactttattaattaaatatttatttatttgctttctatcctgactatagcttccctcgctcctctcctctcagtcctttcccccagcgtgctcccccctccactcctcctctttttctcttcagaagaggccaggcctcctgtggatatcagctagccctggctatgaagttgcagtaagactaggcgcatcctttcctgtttaggctagaggaggcagcccagtagggagaaagggtcccaaaggcaggcaacagggttagagagagcccctgcttctgctattataagttccacatgaagaccaagctatacaactgaaacatatgtgcagagggcccaggccagtcccatgcaggctctctaggtggtgtttccatctctgtgagcccccactggcccaggttagttgattctgtaggttttcctgtggtatatttggcacctctgcctcctatgatgcttcctccccctattactttggattccccaggctctgcctaatgtttggctgtgcatctctgtgtctgtttccatcagctgctgggggaagcctctttgatgatggttaagctaggctcctgcctggaagtgtagcagaatcattagtgtcagtatcagggatatataatatcatatataatatataatatcattagtgtcagtatcagggatatgctcctctcctctgtcatggcctgatatcaagctggaccactccctcaatgtccgctctatctttaccccagcatgtcctgtaggcaggacaaactgtaggttgaagtttttgtggctgggttggtgcctcagtccctccattgaaagtcttacctggttacaggagatggcagatgactctatagcctccattgctaagagtcttagctagggtcacactgggagcttccatttccctaggtttctagctcattccagagatgccccccagccctgattccagttgtctctcctggtactctctccttccattttcctggcacctgattcttcctatttccatcccaagaccctacactcatccagcacacccccatctacccacaaggtctattctatttccccttttcagtgggattcaaacatccccaccttaggccttccttgttacttagcttctttgggtctgaggattgtagcgtggttatcttttactttatggctaatatccacttattaataagtacataccatgtttgtctttctgggtctgggttatctctctctggatgatttttcccctagttctactcatttgtctgcaaatctcatagtgtcattgtttttaacagctgaataatactccattgagtaaatgtagcccattttctttatccactctttggttgagggatatctagattgtttccagtttctagctaatatgaataaagctggtatgaacatagttgaacaagtgtcctcacggtatggtagagcatcctttgggtatatgctcaggagtggaactgggtcttgagatcaagtcccagttttctgagaaactgccaaattgatttccaaagtgactgtgcaagttaacactcacaccaacaataaaggagcactccccacaagtttacactcacacgaagaatggaggggcgctccccacaagtttacactcacacgaagaatggaggagtgctccccacaagtttacactcacacgaagaatggaggcatgctccccttactccacatccttgccagcatgagctgccacttgaggttttgatctcagccattctgacaggtgtaagatggaatctcaaagttgttttgacttttttccctgatggccaaggatgttgaacatttctttaagtgtttcttagccattaaagattcctctgtcaaaacttactgtttagatttgtatccctttttttaaattggatcatttggtttgttgatgtctttgtacttttgaaattatccctaaatgtttgagttttaaaataaacataatgtaatatt

SEQ ID NO: 24 - DNA, 468 bp, Homo sapiens
ccctcctgtcccctccctagacaaagcaatagggatggttaaagagagtccagcagccctcctccacgctgtgcccctgtaccgactggaaggatgattcttgccgcgacccttacccttctgtttgcctacaaggatgtgctgtgcatcacgctgacccagagctccactgaccagacagtggcaagcggcactgaagtaacactgctctgcacgtacaatgcggattctccaaacccagatttattttggtatcgcaaaaggccagacagatccttccagttcatcctttatagggacgacactagttcccatgatgcagattttgttcaaggtcgattttctgtgaagcacagcaaggccaacagaaccttccatctggtgatctctccagtgagccttgaagacagcgctacttattactgtgcctcggggtattataccggagggatacgagctaccgacaaa

SEQ ID NO: 25 - DNA, 1218 bp, Homo sapiens
gagattctgacctcttccgtcaacaccatccaactaattcacctgcgtgccaccatgaagccagtgatgcttacaaatggaaaccacagcttcttctgtgagcactttggaaagagagagcagaaaatcgggctgcctttcgggaatcgtgaggccttacacaggatcctagacctccatcgggagctattaaatgggattgaggttagaggtggggcaggcattaggggaggatcctgaatttacctctctcctcacaatattttctcagcatcgattgccttttctccaggttgtggggaagtaggtggggctcaaaacatattttttttcctactcctgaaatgtagaacttgtatatgtattttaaaggactactgagcagttctagatggaccaatccaattttctaattcaactttataggatgaatgatggaagattactttgactagctctttgtagttctatgaagaaaaccttagcacactgggtcagttcttgggtccgctagtagtttagttacctgttctgtggtgcattttcttaatccagtacatccaggagtgtgctagcctttctttctctctgtgtttcttagcttggaaacaatcttcctcccttttggcactcaaaatggtttcatttacatccagttttatttactcagacaatattgaacttgactatattttgtaccacatcttctgtttggagctgggaatgaatacacaaggaagtcaatcaaaggcagttctagccttgccctcaccacatgcagtcaaatgtgggctacaacagttaagcaggcttgtgacacgaggaaaagactatgtgacacagagctcaagagccttttgggagccctcagggtgtgctgttttccaaccttgcagtgttcggagccagtagctttcttcagaggcagggatgaagtggagccggcatacactgtggcaatcctcctcctctgaaactctgcaggataagagaaatcctctcctctctggactttgggatgtggagaggtcttttttctttggaatagtgtatggcctatctgttccatcgcagcaagagtagcaggttgtcttgggaagacaaaatgtgttactttacagggaagcaggagcagggaggaggctgacatttataaagtaactatattagatacaacatttaagttgccttccggactttaaatgtacccaactcact

SEQ ID NO: 26 - DNA, 1816 bp, Homo sapiens
cttcaagcctgcttcgggctgtgtagccagactgctacaaaggggttaagagagagagagagagagagagagcaagagagagagagggagagcacgcgcgagagcgagagcgagcattcagctggcttgagctgtgtagtctgcagagacttcagaaccacacattccttcttatgtggaattgtgaaaagaacactagatgattttatgatttacatttctacttttattgcctaatttcttttcacacttggaaggagaagggaatttatccatgtttttctacaaactaaaaaaaaaaaaaaaaaaaaacctggcaaaaaatgccatattgcatgttaaaaaaagcattaaaatatggattcattaagaaaatcaagtaggcatacagtaatgtctttaaatgattagtatattttggtggatttgatctttacaattaaaaatacttaattaaaagtttgattctgttgttgttagactaaaataacattttcctaaagagattttaagctgattcaatactgtgaccttttatccttcttttctgtttaatgttctcgctggtgaaagtctttgaagactgttgtctaaattctgtatagtgcttagatacatactttgaagagcaaggagtccattgttgttgattctaaacttgtgttgtatgtctactttgtagacaaaaataaatgcttattgacaaaaatttttctatgtaacaaattttatttagcataatcagctccaacaaccagcataaaaagtttacagagaacagaaagtacattaatgtaagctagtctttacaaaaaggggaaaaatatgttcttgatcaaaatagtttacacttcagtgataaactgagaatgttttttaaaggggactttatgtgtatgggtactttgcctgtatgtacatctgcgcgtcatctacaagcctaatgccaagggaggccagaaagagtatcagatctgctggaactggagtgtgagaggctggtgaactgccgtgtggttaccaggcattgaacctgagttctctaggaaagctgccagtgcttttagccactgaaccatctctccagcccaaaaaagctttaagaagttggtctctgcctgacagcagaagtcagtagaggtcactgttccgtgtgttacctatgcattagtgccctcagctggaactgcctacaggacaggagacagacacagtcaggaacacatcagctctgtcactctacccgtgaaggcttcttctgcctagtaaaagatgtgtcttcatagcatgaagttggggataaattcatctttctcagatctattttccatcttctccagggcagagcttagttcctcgtctttcttcacaagtttgtatgtcagctacttcttgatctaaatgagaccgttccttctagtttaactagctgtgacccattttctccatctgccatctttgtcactgactgccgagagatgctccgatcgtgtcctcacggattgttttgtcacctctgggaccaagagtagtcacaggagccttgagaagcctttccagcagtgagatcacttcagactaagcagtgatgtgaagactggaggtagtgtccatgacacagaactgacacggaagttgtttgctgtggttcctgctctcctggctttcactcagcagtactgactttggctcgcccagcagagaactggaactgtgagcacatggtctcactcactttgcaggtgattcgaagacttaagttggataaaaaacttcgatgaagttataacctttattaaactcaattaataaactt

SEQ ID NO: 27 - DNA, 3073 bp, Homo sapiens
cggactacattgactaagggacaggggcacgatgtactaggctctcaagctcacttagctgtggttatgtgctccattggtcctctgttggcttcctgggctggtatcagacaacgtagggtaaatctctgattgggaggctgctgctgtttgttcgggtctcagagaaccttccctggctaggcaacagaagaagcaagaaatctccacaggtttcagcgctcatgagctcccaagaccttctacttgattggagctgttgtggacagaacaacagatgctgactttggccccaggactctgaaaccatctgactggtgagagccctggatgtacatggatgatgaccgaaaaatccaacggtgtgaagagctctccagctaataaccataaccatcatcctcctccttctatcaaggccaatggcaaagatgaccacagggcaggtagcagaccacagtctgtggcagctgatgatgacacttcctcagaactgcaaaggttggcagagatggatactccacgcaggggaaggggtggcttccgaaggattgtccgcctggtggggatcatcagagactgggccaacaaaaatttccgtgaggaggaaccaagacctgactccttcctagagcgtttccgtgggcctgagctccagactgtgacaacccatcagggggatggcaaaggcgacaaggacggcgagggaaagggcaccaaaaagaaatttgaactgtttgttttggacccagctggagactggtattaccgttggttgtttgtcattgccatgcctgttctttacaactggtgtctgttggtagccagagcctgcttcagtgatctacagagaaactattttgtggtatggctggtgctggattacttctcagacactgtttatattgcagacctcatcattcggctgcgcacaggcttcctagaacaagggctcctggtcaaagaccccaagaaattgcgagacaactatatccacactttgcagttcaaattggatgtggcttctatcatccccactgacctcatttattttgctgtgggtatccacagccctgaggtacgctttaatcgtctgttacactttgcccgtatgtttgagttctttgaccgcactgagacacgtacaagctaccccaacatcttccgaatcagcaacctggtcctctacatcttggtcatcatccactggaatgcttgtatttactatgctatctctaagtccattggctttggggttgacacctgggtttaccccaacattactgaccctgaatatggctacctggctagggagtacatttactgcctttactggtccacactgaccctcaccaccattggagagacaccaccccctgtaaaggatgaggagtacctatttgtcatctttgacttcctgattggtgtcctcatctttgccactattgtgggaaatgtgggctccatgatctccaacatgaatgccacacgagcagagttccaggccaagattgatgctgtcaaacactacatgcagttccgaaaggtcagtaaagacatggaagccaaggtcatcaaatggtttgactacttgtggaccaataagaagacagtagatgaacgagaagtcctaaagaacctgcctgcaaagcttagggcagagatagccattaatgttcatttgtccactctgaagaaagtgcgcatattccaggattgtgaagctggcctgctggtggaactggtactgaagcttcgtcctcaggtctttagtcctggagattatatttgccgtaagggggacattggcaaggaaatgtacatcatcaaggagggcaaattggcagtggtagctgatgatggtgtgactcagtatgccttgctgtcagctgggagctgctttggtgagattagtatccttaacattaagggtagcaaaatgggcaatcggcgcactgccaatatccgtagtctgggctactcagatctcttctgcttgtccaaggatgatcttatggaagctgtgactgagtatcctgatgccaagaaagtcctggaagaacggggtagggagatcctgatgaaggaaggtctactggatgagaatgaagtggcagctagtatggaggtagatgttcaggagaagctggagcagctggagacaaacatggagaccttgtacactcgttttgcccgcctgctggctgagtacactggagcccagcagaagctcaaacagcgcatcacagtgctggagaccaagatgaaacagaaccatgaggatgattacctatcagatgggataaacacccctgagccagctgttgctgaatagccataggtgcctgtccagccttgctttgaccccaggagttagaagtgctgtatagaactttacatttacacacattatgctcatgtccctccgaactcccaaagccttgctgaggcttaaggttttggctacatcttggagtccccttccaagtccagctaacagccaagcttgtggataatggagatcatgtgggttgaatttctaagagcgtgacctcctaagtctcacaaggaatcagagaatagctaaattgtccttcctgaggcttttctgggactagataactagacagtgttctctgaagaacactgtgcaccacaatgcctgcctgactccttttagcttctttatatcgagtcactccctactgcattctgccccaaatacctttttaatgtgttctctgaacagcctgtttccatgcacttgtacacatttaagaattggctgcagacactgggccccctaccctgcctcccaaggcatgcaaggtctgtgaggggagtgggaaggtgggtttgagtgttggtgctcagggtcatacttccttgtcagacaatgtcactctgagaaggaggtggctggcagctttggccatcacaccttatgcacacaagttctgaagagtttgtgaaggctactgctgcatgagatactgtgaattagaggcacttaaaagttaataaattcctttcagctaaaaaaaaaaaaaaaaaaaaaaaaaaaaaaaaaaaa

SEQ ID NO: 28 - DNA, 2537 bp, Homo sapiens
agatttctgggaacgacgcgggttctcgcgagggccacccaggtcctgaaggagttaaaagacagcgaagtctgcaagctgggttagcgttgggacagctcgctcgttagtgtctgtttgcgtcgttagtctagcctggagggtaaaagctgtaccggagtctcttctgttgtaatcgtgtttgccaacacccatgtctcccttatttcccaccctagctgcgaatctccggataaattagatccctcgggaaccataagtgtaaagaacggagcacagtcaaatattgtgaaaagctctttccataaatgtgtgtgtggcgtttttaatgtaggcccgttcttgggcatgcattttttcccccacggttccactttaggatatatatttaaggtattactccattttagcgaggaagcaatagaaacaaacttggtcttgggggctgcagagatggctaagctgttttctgctcttggagaggacttgggttcatttcccagcacctacatggcggcagttccaggggatcccacgtcgcgccaaacaacagaaccttactgcctaactgttctgaaatgcagatgttagttggttgagcagggggccctttttgaagccccaaggagtgagtggacgtcccatgtatcttcagcatctggtgcttcagacgttccttggcttatacctgactcctgcccctgcctttcattctcttccgtctttgttgtgagaacacttgtcagtttgttaagatagcttgcaaataagcttagagactataattccctgtggacactgtctcttgtgtcgctggtaacacatctttaagtgttacggctctgttaggggaaggcttcagctgctgggaaagtttccctgatgcaggtgttacagctttgctctggctcatagaaaattttccctgtgacagttctgctccaaaaggtatcctggcagtcaggagccaaggaataccacaaagtcttgctgcacaactaagctcaggcaagggattaatttggagggaaaagctaaggagggtggttgccctgcttggaagagaagcaacagagaactgggcagaattgggggatggggaaatatatcaggattctggggtggggggttactttccagggtggtctgggattttattttacttttttcttgtacggcgagacctgggctgggaatggctattacagcctttaaaagatgaccattagggaagtcaagggcacatgtgtgtgtgggggggggggtctggctatttcctgccttgaagcactagaaaggcagttacagctgttttagcctttacacccttgctagagctcctgaggccaagacctagccacttttgtgccttgaggatttacaacggctactaagtatacaaaatttacaaagggaggtcatggcagaaaagcctgttgcttgagttggtgtaaatggccagcaatacctatttgaagaaagtggaaaataccacagttgatcctttttagaagtaccatgtgaaggatgtattataacttcccctcaatatttaatattatacagttacaagaggcttatgaaagcaaattccatagatattccatgaaggaacggagttgcagggctctattgtgaccaatcaaggaaaggttataaatatcctgataaagtctggctatattattttgtttcactttaattttaaaaagatcttcaggttttgtttggttgttgttgttgttgatgatgatgatgaaaaaaatagacccaagttgtacctctgttgctgggtatgacttagaacacatatattctaaactgatcagaaaataaaattacaaaaacatatcagaccctccagttaatccagatctgcctgtcaagcatctggacaggaaatgttctggaaaccttaacgaaatataataaacttaaaagcatgttaagatgggagcatgcttgtctcctcagtgagccatgcacttatgaatatccccgaagcttctctcacaaggcagtcgcttacagatgctctctgctctgagaccctgctttcggaattacagctgactccaactcttccacctccctttttatatctccctgctctggataacagcccgatcccaaactggattgccttcactccctggacagttgttttctctgaagatccctccccgactggccatgcactaggctacatgttactgatcctgcctactttcttgactaataatttgaaaaacttagctttcttcttacccagtgctctcttcctggaagctgtcaagagctctagcttgctgaccctttttgttttctcaaacacacacaacatacaccacacacacacacacattgctgttaagatagctcagcaagtgaaggtgcttgccccaggcctgaagacagacatgaatttaatccctggagcccaacttatcctctgacttccatacatgtaccattgtaaatacacacacaatatgattttttaaaagcacccttaaaaatcaataaaagcaaactcc

SEQ ID NO: 29 - DNA, 3863 bp, Homo sapiens
ttttttagaggaattatcatgactgctcccaaatatatatggaaagaatatgatcagatgccaaaactgtgattaatatttaaatattgaataggattcacatccacagcaggatgctttaaggaatggaatgtgttgtagcacccagagtatttgctaagaggctgaggcagcatgcctgtgtctcagtcttcacaggcccaagtcaggccctgatgtcagacagagatgacgtcataggccctagcccactgttgcagagccatctctatttctgctttgaattctgatgggcttgccttggagaggtttcatacctcagctaaactttcttttatcaagataaattatgggcagtggtcagtactcacttgtcactaagaagatacacaatctaacgagtcctagcacagtctgacttttttcttagtcatactagctagtcatacactgcaggatctaagttattttatcctgtgccctgtctgctagtcctcattgttaacagatattcaaagtcagattcacttcattatcatactgtactagaatttgccatcaaaactttctgctttctgctctttggcactgttctctgttttcatatctagaatgttttacccatcgttatcctcatgggtatcaaatcactcacataatttccttgaatatcactttgtgctaagtgtgacttgacaaatatatttggatacgattaagagtctgtgaggattactatagatttggcaatgtctagttggagattacagtaccatgtcaaacctccataacaacaaaactagtaaaaagatttggaattaaaacagagaccatctccaccctagggagttagaaaagccgtgttggatgtcataatctgggctaagtgttcagtaactgtgtagtgctggccagatggagcctggtgtgtgagagcagagacgtgatggcaggcagggtcagacattcatggcgaagggctctgcaagctgcaggcatcatttggtctccattcagttctccacttgcactgcatggggataaatctgacttaaggaagctggcaatgagagccactactggaatgctttcatacacaagctcagccacaaaaggacatgctgacaaccataacaaataaccgaatgagtgatgaatgaactctgctgagaagcaaagaaataaaaacagtttgaggatatgtccaatcatattgtttttcataactgggaactgagtactttttattcatgaagcatggtgctggatgctaaggaagacagtactcctcagtctaagaagaaaatcgcatgttaaagagctcttatacaagtagacatgactgcaaggcttagtaaatgcaagatagataaagtacagggtgccgaggagttggatggagcagttttaattaagtgtaagtggagagccagttagcactttcaaggaagagaaagtctgctgagcctgaaaacagggcacatggtagaaccttctcaggagtgagatgtgaagatgctgaaatggaatagtgctaagcttcaaagacccgagaaaacaggaatgaggaataggaaggatagaacacctgatacaggaggagggtctgaggatggtgggaggtgtttggagggtttagtcaattgaagaggaaattggaagacacgaggaagcatctcgtgtctgctttctgtgtttttgtagaactaacacagatcattaggaggctgggaactccagttctccttcagtgcagtcaggagatgcaagtggagtgacgacactgttttctttttcattttttaaagatttatctatttatttattattagatattttctttatttacatatcaaatgttatcccctttcctagtttcccctctgaaaaatcccctatcctctcccatcctccccctgctcaccaacccatccactctctcctccattcccctatactgcggcatagaacatttaaaggaccaaggtcctctccttccattgatgaccgactaggccatcctttgctacatatgcatctagagccatgagtcccaccatgtgttttctttgattggtggtttagtcccagggagctctgggggtactggttagtatatattgttgttcctcctatggggctgcaaacctcttcggctccttgggtactttctctagctccttcattggagaccctgtgtactgtctaatagatgactgtgagtatccacttctgtattagtcaggcactggcagaacctctcaagggacagctataccaggctcctgtcagcaagctcttgttggcatccacagtagtgtctggttttggtggttgtttatgggatggatcccctagtgggacagtctctggatggtcattccttcagtctctgctctgaactttgtctctgtaactctgtccacgggtattcttgttcccccttctaagaaggatcaaagtatccacactttagtcttccttcttcttgagtttcatgtgttttgcaaattatatcttgaatagtctaagtttctggaataatatccacttatcagtgagtgcatatcttgtgtgttcttttgtgtttgggtatcctcactcaggatgataccctccagatccatccatttgcctaagaatttcataaattttaataactgagtagtactccattgtgtaaatgtaccacattttctgtatccattgctctgttgagagacatctgagttctttccaacttctggctattataaataatgctgctatgaacatagtgaagcatgtgtccttattacaagttggaacatcttctgggtatatgcccagaagaggtattgctggatcttccgagagtattatgtccaattttctgaggaaccaccaaactgatttccagagttgttgtactagcttgcaatcccaccagcaatggagaagtgttcctctttcaccacatccttgtcagcatctgctgtcaccttagttttggatcttagccattctgactggtgtgaggtggaatctcagggttgttttgatttgcaatttcctgatgactaaggatgttgaacatttttttaggtgcttttcagccatttggtattcctcagttgaaaattctttgtttagctctgtaccctattttaaaatatggttatttggttttctggagttttatatatattagatattagtatgaaactattttctacactaagaggacaaattagaagaactcatcactttcctttctggatatggcgtcacaaagctttaatcccagcactgttaaagcagaaactgagagatctctatgaagtcagcctgatctacatagcaagttcccgatagccaaggctacacaataagatcttgtctcaaaacaacaagaacaagaatgacagttccatgattcataagagtcttttctgaggcttgctttatagtcatttcctctaacataaagcagaccatattggaaggaagctgcttaagctaagaacctaaacaactcaaaagctttaggaaattcctgaaactgatcaggctcactagacaccaccccctacttactcacgcttataaaaccagtataaactactgagagatactctcagacaagtagagctgctgagacgaagctcagattacaggtggtgactgaaagagatgctcaacaatggaaaatagacattttccaagatgccaagctgccaacaaatgcactgcaggtttctaattttcataaactcatgcagatgtgggtttttgagatgtagctatctttgagtcatttttgatcctgtaattaatccttcaatcatactcctgtaagtaatcccaataaaacacactgcttc

SEQ ID NO: 30 - DNA, 4494 bp, Homo sapiens
tgggcgctttggcagtggcggttccctcgggactgcggggaaggcccaggccgccgccgcctgctcagttctccctcactgcgtctaaggctctcccggcctggctccgcgcccagcccagactacgggagggggaacgtggaggcgacggaagagcccgtcgcgcctggggctcccgaaatacaaccagagacctacagagggcagcaccgagccgtaaacgggtcctccgcactgcaagcttggggtcgccagactgcccaaagccaagtccccctctttaggacagggcagggttcgtgcccgaccagtccctggcctggataaaagtcaggaatgtgtctaaccatcacaagaaccaacagatcctggcggggacttaggactgacctagaacaatcagggttccgcaatccaggtcccaaaggtgggatcctcaaccgcaggacggagggaatagcctttcgattctgggtggtgcattggaagccccaggctctaaaacccccaacctactgactggtggccgagtatgcacccgactgctagctaggcagtgtcccaagaaccagtagccaaatgtcttggcctcagttttcccggtgacacctggaaagtgaccctgccattagtagaggctcaggtcagggccccgcctctcctgggcggcctctgccctagcccgccctgccgctcctcctctccgcaggctcgctcccacggtccccgaggtgggcgggtgagcccaggatgacggctgtagaaccccggcctgactcgccctcgcccccgcgccgggcctgggcttccctagcccagctcgcacccgggggccgtcggagccgccgcgcgcccagctctaccggcctggcgccctccccacgcgggcgtccccgactcccgcgcgcgctcaggctcccagttgggaaccaaggagggggaggatgggggggggggtgtgcgccgacccggaaacgccatataaggagcaggaaggatcccccgccggaacaggccttatttccccagcgccttatatggagtggcccaatatggccctgccgcttccggctctgggaggaggggcgagcgggggttggggcgggggcaagctgggaactccaggcgcctggcccgggaggccactgctgctgttccaatactaggctttccaggagcctgagcgctcgcgatgccggagcgggtcgcagggtggaggtgcccaccactcttggatgggagggcttcacgtcactccgggtcctcccggccggtccttccatattagggcttcctgcttcccatatatggccatgtacgtcacggcggaggcgggcccgtgctgttccagacccttgaaatagaggccgattcggggagtcgcgagagatcccagcgcgcagaacttggggagccgccgcgattcgccgccgccgccgccagcttccgccgccgcaagatcggcccctgccccagccctgccccagcctccgcggcagccctgcgtccaccacgggccgcggctaccgccagcctgggggcccacctacactccccgcagtgtgcccctgcaccccgcatgtaacccggccaacccccggcgagtgtgccctcagtagcttcggccccgggctgcgcccaccacccaacatcagttctccagctcgctggtccgggatggcagcggccaaggccgagatgcaattgatgtctccgctgcagatctctgacccgttcggctcctttcctcactcacccaccatggacaactaccccaaactggaggagatgatgctgctgagcaacggggctccccagttcctcggtgctgccggaaccccagagggcagcggcggtaatagcagcagcagcaccagcagcgggggcggtggtgggggcggcagcaacagcggcagcagcgccttcaatcctcaaggggagccgagcgaacaaccctatgagcacctgaccacagagtccttttctgacatcgctctgaataatgagaaggcgatggtggagacgagttatcccagccaaacgactcggttgcctcccatcacctatactggccgcttctccctggagcccgcacccaacagtggcaacactttgtggcctgaaccccttttcagcctagtcagtggcctcgtgagcatgaccaatcctccgacctcttcatcctcggcgccttctccagctgcttcatcgtcttcctctgcctcccagagcccgcccctgagctgtgccgtgccgtccaacgacagcagtcccatctactcggctgcgcccacctttcctactcccaacactgacatttttcctgagccccaaagccaggcctttcctggctcggcaggcacagccttgcagtacccgcctcctgcctaccctgccaccaaaggtggtttccaggttcccatgatccctgactatctgtttccacaacaacagggagacctgagcctgggcaccccagaccagaagcccttccagggtctggagaaccgtacccagcagccttcgctcactccactatccactattaaagccttcgccactcagtcgggctcccaggacttaaaggctcttaataccacctaccaatcccagctcatcaaacccagccgcatgcgcaagtaccccaaccggcccagcaagacacccccccatgaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcgagaagccttttgcctgtgacatttgtgggaggaagtttgccaggagtgatgaacgcaagaggcataccaaaatccatttaagacagaaggacaagaaagcagacaaaagtgtggtggcctccccggctgcctcttcactctcttcttacccatccccagtggctacctcctacccatcccctgccaccacctcattcccatcccctgtgcccacttcctactcctctcctggctcctccacctacccatctcctgcgcacagtggcttcccgtcgccgtcagtggccaccacctttgcctccgttccacctgctttccccacccaggtcagcagcttcccgtctgcgggcgtcagcagctccttcagcacctcaactggtctttcagacatgacagcgaccttttctcccaggacaattgaaatttgctaaagggaataaaagaaagcaaagggagaggcaggaaagacataaaagcacaggagggaagagatggccgcaagaggggccacctcttaggtcagatggaagatctcagagccaagtccttctactcacgagtagaaggaccgttggccaacagccctttcacttaccatccctgcctcccccgtcctgttccccttttgacttcagctgcctgaaacagccatgtccaagttcttcacctctatccaaaggacttgatttgcatggtattggataaatcatttcagtatcctctccatcacatgcctggcccttgctcccttcagcgctagaccatcaagttggcataaagaaaaaaaaatgggtttgggccctcagaaccctgccctgcatctttgtacagcatctgtgccatggattttgttttccttggggtattcttgatgtgaagataatttgcatactctattgtattatttggagttaaatcctcactttgggggaggggggagcaaagccaagcaaaccaatgatgatcctctattttgtgatgactctgctgtgacattaggtttgaagcattttttttttcaagcagcagtcctaggtattaactggagcatgtgtcagagtgttgttccgttaattttgtaaatactgctcgactgtaactctcacatgtgacaaagtatggtttgtttggttgggttttgtttttgagaatttttttgcccgtccctttggtttcaaaagtttcacgtcttggtgccttttgtgtgacacgccttgccgatggcttgacatgcgcagatgtgagggacacgctcaccttagccttaagggggtaggagtgatgttttgggggaggctttgagagcaaaaacgaggaagagggctgagctgagctttcggtctccagaatgtaagaagaaaaaatttaaacaaaaatctgaactctcaaaagtctatttttctaaactgaaaatgtaaatttatacatctattcaggagttggagtgttgtggttacctactgagtaggctgcagtttttgtatgttatgaacatgaagttcattattttgtggttttattttactttgtacttgtgtttgcttaaacaaagtaacctgtttggcttataaacacattgaatgcgctctattgcccatgggatatgtggtgtgtatccttcagaaaaattaaaaggaaaaataaag

SEQ ID NO: 31 - DNA, 1240 bp, Homo sapiens
gagagtgccagtctccattgcaaacagtggcccttgaaagagcaagcttcaaggaaggacaggcagggggggcatcctgaagcctgtcagaaccgtgggccttatccaaagggaaaaagtagctgtcacttgttagagcagagtcctcggcacagtgtgtccgtcctgtttggaagtgttcccaaaaggactcgtgagctctgtgccatcaagtacttcccttcatcaagctgaggaggccacagcctccgagaggaactggtgagtgcttatggagcaaagagcatgtagaaccttctagtctctgaccacgtggtaagatggagcatctgtctcagcaagctttgctctctcttttctctctggcctattctggtcccctttctgcaagagtttgattttccctctcttttctctacatgtctgcaggtgtgtcagtacatggatggatagacaaacacacacgctcatgcacacatacttaggcacatacacacttagatacacacttatacacacacacacacatgctcatgcacacatacttaggcacatacacactcagacacatgcacacactcactcacatacactcagacacacactcacacacacacatgctcatgcatacattctcaggcacacactcagacacacacacactcagacacacacattcagatacactcagacacacaaacacactcaggcacacacacactcacacacacactgagacatgcatgtcccatgtggggataggttgcacttcctctttcagctgtggcttctgggcagctgctctactctgtcatttctgccttatgaagctttgtccctattcaagtaacagaaagcagaaagtggcccgggaaactcgaccttcaagcacacagaacaaaagagagagccactgtcttcctggaaatggtgcatcaaaactatttgctgggattggcttagagctggcttgatggctatggtctatttttggatgggtcaagctggtataaaaaggatgtgtgaacgtgaagatgaagaaatgcactttctatggacagaactaagactcacctgaatgagtctaggaaagaaacttaactcagttacaaaatgctggataaagccaagagacttaataaagtactttcttgtcaaaaaaaaaaaaaagaaaagagaaaagaaaggaaggaaggaaggaaggaagaaagaaagaaaggagagaaagaaagaaagaaag

SEQ ID NO: 32 - DNA, 1382 bp, Homo sapiens
gagccgaagccgccgagccgccgcccgcagccgtggtccctacagtcttcatggtccttgacgaggctcaccactgttcagtgtcctgggttaagaaccacactttcaacctaagtgccgctgagcagctggctataagcatccccaacgacacagcgggcagacccgagtcctgcctcatgttccggccacctcctgacagtgccagcctggaagacatcctgagccaccgcttcaatgagacacaggcctgtgactcaggctgggactatcctgagaaccggcctcagtccctaaagaatgagtttgacctggtgtgtgattgaaagaacctgaagaggacctctcagtcggtgttcatggctggacttcttgttggagccctggtctttgggcctgtctgtgactggtttgtggacagtctggtgtactacagcctcagcttccaagtgggggactttggcctggatatctatgtgacacagctgatatttggagcagtggaaatgcctggccgcttcctcagtgtcctcatgatggagaagctgggtcgcaagtggagccagctgtgtactctgactctggctggcatcatgtacatcattatcatcttcatcccaggagatcttcccacggtggtcactgtgctggctgtggtggggaagtttgcctcggctgcggccttcaccatctcctatgtgtacactgctgagctcttccccaccatcatcaggcaaacaggcatggggttggtgagcatgttctccagggtcggtggcatcatcacaccccttgtgatgctgctggaacagtaccaccaggcgattcccatggtcatctttggcagcctccccattggagcaggcctactgtgcgctcttcttcctgagtcccggggccagaccctgaaggacaccctccaggacctggagcaggggctccctgcagggtcccacaaaacagcaccccaaagacaagacatggaggcttcgggaagaacttccagtgtgagggtggctgttgtgaagagctcgtacttctgactgtggctgctgagggccagcctatcaagagcagaggacaggtttctgttttgtcacacaagctgcattcccagacctgcctttggggtctttgtaggcattttctcttccatttcttaggggttatctatgtctaggagagatagttccattctgtaatgcttaattttcattgtcaccctgaccggatgggaatcgctaaaggaggagcatctctgggtggtgtctgtcaaggtgttgtcagaagtttaactgaggaggggagactgtccctgaacatgggcagtatcctattggctaaggtcctgaactaaataaataaaaccatgaagggaaccg

SEQ ID NO: 33 - DNA, 927 bp, Homo sapiens
atggccaatctctccacagtgtctgtgttcattctccagggcttctctgctgttcctgcattacagttgctgagcatggccatctttcttctcatatacctggctgcggttctggggaatgtctctatcatgatagctgtgactcttgactcccatctgcacacacccatgtacttcttcatcaaacacctctccctagtggatctctgttctacatccaccaccctgcctcgggccctggtggctaccatggcagacaccaaagagatatccctcccagcctgtgcatctcaactctttgcttttgtctgctttggctctctggaatgttttctcatcactgccatggcttttgaccgctgtctagccatctacagacccctgacatatggggtaaccatgagttctcagacctgtgtgtccctggtagtggtggcctgggtcagtggactcctcttttctaccttccacatggtcaacactttctccttgcccttctgtggtcccaacatgattgatcacttcttctgtgacatccccccactcatgcatctagcttgtggtgacacccaaggccatgaggctgcaggttttatagttagtggatgtgtcatcatgacttgctttgccctcacttgcctgtcttatgttcttattgtatacacagtggttcacatccgttctgcagctggccgctggaaggccttctccacgtgttcctcccatctggccacagtgcttctgttttatggcactggaagttcagcctacatgcagcccacagcccactactccccactgcaagggcgcatggccgccatattctattctatcctcatgccaactctgaatccacttatctacagcctcagaaacaaggacatgaaagcagctctgcggaagctttatccacaggtgccatcctag

SEQ ID NO: 34 - DNA, 2808 bp, Homo sapiens
gcgcttccctccctccggggctgggcctgtcccggccgtcccggagtctctgtcccgccgcgccgttagctgtctgtgtcttggccaccgcctccaggcagtccgcagcaagccagcttcttggtccgacgagctcagcgccctctcaccgcgatgctgtgcttcctcaggggaatggctttcgtccccttcctcctggtgacttggtcgtccgcagccttcatcatctcctacgtggtcgcggtgctctctgggcacgtcaacccctttctcccctatatcagtgacacaggaacaactcctccagagagtggtatttttggattcatgataaacttctctgcatttcttggcgcagctacgatgtacacaagatacaagatagtggagaagcagaatgagacctgctacttcagcactcccgtttttaacttggtgtccttggcgcttggattggtgggatgcatcggaatgggcatcgtagccaacttccaggagttagccgtgcctgtggtccatgatggcggtgcgcttctggctttcgtctgcggggtggtgtacacgctcctgcaatcgatcatctcctacaaatcctgtccccagtggaacagtctcaccacgtgccatgtcaggatggccatctccgctgtttcgtgcgcagctgtcgtccccatgattgcctgtgcttcactcatttctataaccaagctggaatggaatccaaaagaaaaggattatatatatcacgtggtgagcgccatctgtgagtggaccgtggcttttggttttattttctatttcctaacattcatccaagatttccagagtgtcactctaaggatatccacagaaatcaatgacgacttttgaaagatcgagaatcctgtctcattcagggagtgtcgcagacagtttctggaagtggacagaggacggacgggcttggatgtcaccctgatggggactttatctgtggcacatccgggacttgaatttcattaagagttcctagtagttcaatttacaaaggtatgtttccctggaggatggatagcaccaacgacactgtagcaatatttttatattttctaaaacaatcttttatgaacaaattcatatgcaaagaagacgaggcattgcagaaaggggaggattattcttgtatagattctttagactttttatgtaataatgatttatgaaaatacactaagagaaaaaaatgttaagtttagtactttttattaaagaagccaaatcagggcatattcactttaaaatttcattttttaaatacagtgacctgcatacattttcatcacaagagcacttatacaattcaattcatagtgattatataccctaatggtatagatttaggtaataaacgaacactttaaagcactctgaattttcagtgcattaaacaaatgcttttatagtgaaggactcaaaaccatttacagtgcacattaaccagcgaatgtggaagacctcggttcgaaaatttagccccctcatttacttctccaagggacccacagctttactgccgctgttaatggtgggcccgggaactaattccaggtaggatgggctcatcaggccagcttagaaatgatcaaactgcccttctctgtgaccgggcagcacaagttcaattcacttctcagtttccctgtaagccaaagagaatgcagatccaagtcagggagaaaggagagcgctcatagaaacttccagatgtgggctgctgtttatctgctcctatcaatgcctgtgggccactataagggagtcaggccctttccgaagcaaggcctggagactctacctttcatgcagttcaccaatggcaaagaaacgccagctgttgtggaggaagaagtggataacaagaccccccagtctgggccagtaggagctgccccaagggcacgttccaaggacagagaccatttggacagccttagtgaaagggtacctggcgatgaaggggcacacagcagtgcctcctttggctgctaagtgaagggtgctgctcagcaggcacttctgccatgacatcctgtgtcttcttcctcagtgtgcagtggtggcaaccaacgtccatttctgagtcccctgtaacttttcacagtaccgaacatgcccattgtaacactggaacagaaagacagtggctgtcattctgatgagtgagagaggagtcgatacatttcctgtggaagctggggcggactgaatgcatgctgttctttgctttgagcgggagccttggttgatgccttcccagaatgcacttggctctcttcgcttccactggagacccgaccacgtgcctttacccatagtggaacacagtgccttgtggcatgcaataggtgcttaataaatactcattgaatgtatgcacgcataaatggatgaacaagtaacgactagggatgtttgaggtgctaagggttttttttactctagttcacgggtattctaaggtcaacagctagtctgtgccaattagtaatgttgtctgttttgctttgtgaactattctcgtttccacctgttccagctgtgtgagcttcatgattgtgtgacaactctctcctggacagatagcacagaaattgttacatgggctaaacctgtcttggcaaaccgaggaggcccccaaatcacactctgcagattccatgcgacttctagttttatccctgttttggtgttatttttaatttctacaaatatgtatttccttggactttgtacccgagaaagtaaaataaaatatttctttattttagc

SEQ ID NO: 35 - DNA, 1784 bp, Homo sapiens
tcctcatgtcatctgtgtcttaactgctgagccatctctccagaccagcactgagtttcttcatcaggctcttccaagcatctgctctaggcacggagaataaagaactagctccctgcctctagttctggatgttagctccagttccaagacagccaactgaaggaaggatcctaagtattgcaactcgagtgaggtctgggcacgatgtcatggacctcgaatcccagtactaggaaggtgaaggtaggaagatcatgactatgaggccaacctgggctacatagagagttccagactaacctgatctatatagtgaaaccctgtctcatagaatgaaacaacatgaaactggacatagcaattaagaacacttgttgctcttccaggggacctggggtcaatgcccagcactcatggtaacttgcaaccacctgtaactccagttcagagcatctgacaccctcttctgacctccaacacaggcatgcacatggtgcacatacagacagacaaaacatccattcacataaaatttttttttttttttggtttttcgagacagggtttctctgtatagtcctggctgtcctggaactcactctgtagaccaggctggcctctaattcagaaatctgcctgcttctgcctcccgagtgctgggattaaaggggtgcgccaccaccacctggcaaaaataaatctttaaaaaatgtaaaaataaacaagagccccaagagccagatgtaggaagtggaagctggcacaaggaagtgcccagctgggcatgggttagaactgtgaccaacaagaataacaagacagaggcagctttagccaacctactgggacagcagcagtcatggaggtgccaggcttcattctgaccagcttggtgacataccagtgtctgtgtttatctggtggataggtgtctgagactccttaccattctttcttggcaatttttgattgaatgcactaggattctcaggctagcccttaatgcagtgtgcttattacagctgtggaacatgtgtgcacagacccaggagagcatagggagatacgagccattgtgacagtctggtgcacgacaagatgtcagcctcaccttccatttaaacagttggtgagagactttcatagtatgtaactgagtcggggctcgcctcattttaactggaagaagccgccagagcactgtatagaatccttctcaacgatatgggaatgcaaggtgcaaaggaattcttgctgcactgtgggatgtggattagcttacagagtgcttgcctggcatgcaagaagccctgggtttaattcccatactaaataagctgatgatagtggtgcatacctattatcctagcactcagtaggcagaggcaggaaaatgagaagttcaagattgcctttgattacatagtgaattcaaggaagtctgggccacatgagaccttacctttaaaagatataagtaaaacatgagttggaagccaacctggactaaatagtgatcaccaggtcaactagagtcacgcaacaagaccctgtctcaaacagactaacaagaaactaaaatggctgatgaggtagcttagtgggtaaaggtgtgtgctgccaagtctgacaacctgagtttgatcccagaagccgcacagggaaggagagacaactctcgactctggaacgttttcctttccttcagattaatgccatggtgtgtccacaaatgtatatgcaaatacgcattaagtgtaaataaacaaaaccctgaaagc

SEQ ID NO: 36 - DNA, 948 bp, Homo sapiens
atggaagtctgtaactccaccttgagaagtggcttcatcttgatgggtattttggatgacaatgattttccagaactgctctgtgctactatcacagccctgtaccttttagctttgacaagcaatggactactgctgctggtcatcaccatggatacccggctccacgtgcccatgtatcttctgctctggcaactctctctcatggacctgctcctcacatcagtcatcactccaaaggccatactagattatcttctcaaagacaacactatctcatttgggggatgtgcccttcagatgttcctggctttgacacttggtactgcagaggacctccttctgtcctttatggcctatgacaggtatgtggccatttgtcatcctctgaactacacaattctaatgagtcagaaagtctgctgtctcatgatagccacttcatggagtcttgcatctctcagtgctctaggatatagcatgtataccatgcaatatcctttctgcaagtccaggcagatcaggcatctcttctgtgagattcctcccttgctaaagctggcctgtgcagacacctccacatatgaactcatggtttatctgatgggtgtgaccttgctctttccagctcttgctgccatcctggcttcctactcactaattctattcactgtgctgcacatgccctctaatgagggcaggaggaaagcccttgtcacctgctcttcccatctgactgtggttgggatgtggtatggaggtgctattgtcatgtatgtattacccagttccttccacagccccaaacaagacaatatcagctcagttttttatacaattttcactccggctttgaatcccctcatctacagcctaaggaataaggaggtcactggagctttgaggagagtccttgggaaaaggttgtcagtacagtctacattctag

SEQ ID NO: 37 - DNA, 2105 bp, Homo sapiens
acttaacttgactctcaagccatgtctgcttatgaagttcactgccacagtaagcaatagactcattcaaaatggcgggcgtctggtcttcacttctgcctgcaaatgacaagattcttttaaactgtttttctggaggttaacaaatgcccttctgcaggttggtagttttaggctattccagtaaaacacacagtaacaacatagaaactgaattttatcaactagatttactttatggagtgaaaaacaatctgacatagcaagcaattgaaaatctcatgtgtgtatgtgcatctgtgtgtgcctgtgtgtgtgtatgggtgtatctgtgtgtgcctgtgtatatgtatatctgtgtgtatctctgtgtgtctgtgtatattgtgtctttgtacatatgtgtgtgagtgtctgtaccttaaaaaataatcccattctgaaaatgcattcttagtaacacttatttccagtaacgtatgggaagtgttttctagggaaatatagaggaagtgacgacaagatcacttatattcctcaagtacttgagaatgttagctatagaacaagacttagtacattgtcaagcatccagattctgaaagatgtgttagatataaatgagatttgagagagcagggagggaggaaaggaaggagggaggaagggagggagaatatgcaagtagctgtctggtattgtgtaggcacattcctggtcagatggtggctaggtgcagtatcactgagatattaaaaatctacaatgtgatctctgccttcaggatatctatccccatgaattgaataaattgctctcaaacacttagcttttaatttgcaatttatctcacagtcgaaactaattttaaaattaaccttctagtcccaagaatattctttcacactctctacccttgtaaagggacaccgattgtcacttgtttaaaaatggagagacttggatctctttgctcatagttcatcaactgtattctatccgccgccacagagttatttgttataattctttactcttcaataatgggtaattagtgcagaaagacctagtgcggtttgatgtggaagttaaaagcctcaagaggattccttaaccatgcacctgcctcagctggcttatcccagctcagtttggggtttaatgaaaaacatatttttgttgttatagctgagtctcctattcttgacgccacccctcattaagtggggttcccctagagtgccacttggaaaggtatctgttgttaggcgagaggcaggggacttaactaatggctgaagcatgcattctacaacatcataggacaggcatggtgaatagttttacacacacacttggggactactaaatagaggagttgatacagttttgtggaatttgcaatttttataaaacatgtaaataactatataaatatgttaccaaggttcttcctaagcattggttacaatgaaacttaaatgtgcctaatactaagatctttccatctctgactcatggggtaatgcaaaattgtttttaaaatagtaacttttcatttgaaaggtactttatatcaggctcaaatttagtacctatttatatgctgagcaataggggtgggcacatacatcaggagctatgaagtgttatacttcgcttgcctgctgcttgaagaatctgaagaaagcatatgaaagtaggacctgacttgggaaggtgtgtcacagactacagagtcagccttttgggaagttttcagtgtcttcagtattaaaaaaacttgtatgtcttactgccatttgctgttgatcagtagcagtgatttgaatacactattgcacagacgtctgatggctgggatgggaaccatacttaaaatccaatgggcagagccagctctgtcaccctagctttgttggtcttgaaagagatctctaaagttgagtgtcagttaacagactgttaactgttagttgtgattgcttcttcatccacaagaatagagtagctgtttggttgatcatatttccattcttaaaatgtacactgaaatagtttaagatgtattgattaaacatagcatatttatgactagagttttc

SEQ ID NO: 38 - DNA, 787 bp, Homo sapiens
gagagagagagagaagagagagagagagagagagagagagagagagagagaaagagaaacccagggacctcctaataggccccatgtcttcaaatcctgcctctttaaacatgtgaaccattatcactagggtacactcaagtgctagccactccatactacatccctgttctggagatacccacaagtttttacagatcgggaaactgaggctctgagaggtggggtgagaggtatagagcttatggcacagtcaggatcagaaagtgggatgtcacctgaaagcagccacttggagcaaaggcagaggaccctgggagctccctggatagggtccatacacacacagacacacacacacacagacacacacacacacacacacacacacacacacacacacacacacggagacaggggaggggggaggcatcaatgtggagaagggacctgtggccagccttggagaggcagtggctggtgagtgtgtgtgggtgtgagagggtggaaagggtatatcatagagcaatctctatgaaagtgtcgtgtggggtgagtgaccgttctggacacccctctaatatagacttttgagcagaggagtgcctggcccaggagtacctgggagtggggcttgagggaatgaagctggctggtaatctccacctctggctggagcctgcaagtccttggaggccccagagcctcatgttctcagagtggggccagggagggaggggcttctcggcagctggcccaggggccatggcggaaaaacaagccgtgc

SEQ ID NO: 39 - DNA, 1289 bp, Homo sapiens
tgatcagtataggttgtgttcaaataccagtcgttttattttattctttttgaggtagtaagtattaaattcagtcacacgccggcccttttaaagagttttattaataagctggagagatgcccacagttaaagaccactggctgctcttccagaggacagttttcagtactcacaagggatcaccaccatctttaactccagttccatgagatctgatgccttctgctctccagaggcactgggcaggtacatggtatacatacatgcatacatgtaggcaaaacacttatacatataaaattatttaaaaagatttaagtatagggtttcatgcagtccaggctgacctcaaactctctctgtcatctgatcttcctgcctcctctcctcaggttctaggagactggcatgctccactaggcttgatttttttttttttaatgctgtgctttcgaatgacctcagagcttggtccatgctaggcgagcactgtgccccctgagccacatctcccagtctctctaggaggggggattttttttttaatagaagggatttgagcatacgtagatttggtatgagaggtgtccctgaaatcagtccccttgagggtatagaaggacatcatatatcagggatgtccacacttgttgcttttacaccagacaaaactgggcactgaattaacacgtctccatttgttcagccacacttccagcacctgcctctgtcagctggcaggtgtgaggaggtagcccggaagcagagcccgtggaaagatcttttaaaattaatgaacataaatacatctggaccaaaacaccgtttaggcacctttgatgttgagatctaaagcgcccgtctcaaattgtcaaacttttgagagctgacataactctgtgactggaacattccacgctcgaactcctgtgtgacaggtggtaaaatgtggctgtagcaaaatattgtatgaaattaccttcagaccatgtgtatgaggaatatacatgaaaaaaaaaacataaatgagttgaatgtttaaacctggctacgattccctagctagcttgctgtgtaagtgaaatatttcaaaatgataataataataaaaagaatcccacaaatgtaaaacatttgtggtccaagcattttgtaaaacctctgctaaacctgtatgactttggtaaaaactgtaaacaaaacaaaacaaaacaaaacaagacagaacaaaacaaacaaaaccaaaaaccaaaaacaaccaaaaacatcaaaaaataaaacaaacc

SEQ ID NO: 40 - DNA, 1248 bp, Homo sapiens
gacacaattctaagaggagatgttggcagagctggcgtcttctaagccctctgtccttggcagatcgatatcttcttccagcattcccctgccatgtcattcatatggccaactcatctgctccagattctgtagcatgcccaaggtgaaacaaactagataaaacctgtactccggacaatggaagctccctcatcttgcgccacacaaaatagccactccccaccgtctggtgatgtctggaatctgtgtgaagtctcacaagagataggaaatctaatctaggaaggaattacaggcacctgccacagaacaactcaggggtaagaatgtgcttccggcacggtttggtcaagaagctcacagctgtggagcactgcctgggcttctcgtccattccttcctctctcccctcctcacagtgttggctgggtgtgcccacttggagctgcgatggctgctgagtcccaacactccacttcttcaccaccctggaggagcctgaagcagctttggagctctgaggtagacaagcacaccttctcagaggtgcttggacatcctatgcaggaagctctgaagagtcttccagcccgaggctactgtctaccttttcctccaagattgaaacatttcatcttgcagagaaggggcttacgcgaatggtcaactcagaatggtttcaggaagtccacgaccctaaccaggttcttgtagctccttcttgccaagtcaagctgctgagagcccctctcagacaggacaagccgaaaagaagaccagaccagctgcctggaaggagcagacacccgctgaccggcctggaagaggtttagcccaactgagtcacctgaagagagtactcatcatcctgctgagctgcctgtcagccatgcagtccttccagattcccagctctgtgagctgtcacccaggctgcggtgggcttcggtgaggcagctgtctttgagtcacttctgttcctgtcatggatggtcctgactacccaacttctcaagcatccaatgtcctcagagaaagggagaccagagcctgaaacctgcttgctcaggggtggagcttcccgatcaatcgttctgccacgcccactgctggaacctgctgctttgctgcttggagccatactcgtggtcaccctgctactggactctgagattatttggtgggaatcgggccccttcccctgcttcataactgcgtgcagaacagtaaatttgagctttgatc

SEQ ID NO: 41 - DNA, 1833 bp, Homo sapiens
ggacgccccaggacacgactgctttcttcaccacacctctgacaggacaggacagggaggaggggtagaggacagccggtgtgcattccaacccacagaacctttgtcattgtactgttggggttccggagtggcagaaagttaagacgactctcacggcttgggttgaggctggacccaggaactgggatatggagcttctatcgccgccactccgggacatagacttgacaggccccgacggctctctctgctcctttgagacagcagacgacttctatgatgatccgtgtttcgactcaccagacctgcgcttttttgaggacctggacccgcgcctggtgcacgtgggagccctcctgaaaccggaggagcacgcacacttctctactgcggtgcacccaggcccaggcgctcgtgaggatgagcatgtgcgcgcgcccagcgggcaccaccaggcgggtcgctgcttgctgtgggcctgcaaggcgtgcaagcgcaagaccaccaacgctgatcgccgcaaggccgccaccatgcgcgagcgccgccgcctgagcaaagtgaatgaggccttcgagacgctcaagcgctgcacgtccagcaacccgaaccagcggctacccaaggtggagatcctgcgcaacgccatccgctacatcgaaggtctgcaggctctgctgcgcgaccaggacgccgcgccccctggcgccgctgccttctacgcacctggaccgctgcccccaggccgtggcagcgagcactacagtggcgactcagacgcgtccagcccgcgctccaactgctctgatggcatgatggattacagcggccccccaagcggcccccggcggcagaatggctacgacaccgcctactacagtgaggcggtgcgcgagtccaggccagggaagagtgcggctgtgtcgagcctcgactgcctgtccagcatagtggagcgcatctccacagacagccccgctgcgcctgcgctgcttttggcagatgcaccaccagagtcgcctccgggtccgccagagggggcatccctaagcgacacagaacagggaacccagaccccgtctcccgacgccgcccctcagtgtcctgcaggctcaaaccccaatgcgatttatcaggtgctttgagagatcgactgcagcagcagagggcgcaccaccgtaggcactcctggggatggtgcccctggttcttcacgcccaaaagatgaagcttaaatgacactcttcccaactgtcctttcgaagccgttcttccagagggaagggaagagcagaagtctgtcctagatccagccccaaagaaaggacatagtcctttttgttgttgttgttgtagtccttcagttgtttgtttgttttttcatgcggctcacagcgaaggccacttgcactctggctgcacctcactgggccagagctgatccttgagtggccaggcgctcttcctttcctcatagcacaggggtgagccttgcacacctaagccctgccctccacatccttttgtttgtcactttctggagccctcctggcacccacttttccccacagcttgcggaggccactcaggtctcaggtgtaacaggtgtaaccataccccactctcccccttcccgcggttcaggaccacttatttttttatataagacttttgtaatctattcgtgtaaataagagttgcttggccagagcgggagccccttgggctatatttatctcccaggcatgctgtgtagtgcaacaaaaactttgtatgttttattcctcaagcgggcgagtcaggtgttggaaataaagacgctaatttata

SEQ ID NO: 42 - DNA, 993 bp, Homo sapiens
atgcctcgaggtcaaaagagtaagctaaatgccagggagagacgccaccgtgccagaaattatgctcaggcttctgaaggtgctgaggaaacagtagtggcaacagaagagtcctctcctaaatcatgtggcacctctgagatgccagaaaaaaaatcagtttccacagtaactgttgatctcttttcgatatcagatgacagttttattgatcttgaggatgctgaagacctttttagggattaccacactattagtcaccaagatattctgacccaaaaggtctttgtgctggtgcagatcctcctcaaaaactacaaaacaaagcagcttactacaatagaagatatgatgcaggttattgatgaggaggaaatggatgacttccccgagatcctcaggaaagctgctgaaagacttgcagatgtctttgcagttgaactgagggaagtcgaatcaagccggcgagtgtatgatcttatcagtaagctgaaacttcccaacaatgggagggttcgtgctggccagggcttcccaaagactggtttcctgatgactgtacttggcataatcttcatgaatggcaactgtgccggtgaagaagacatctggagaacactaagatctatgggtatatattctggaaagaagcaccaaatctttggagagccccggaagctcatcactcaaaatttcgtgaagctaaagtatgtggagtgccggcaggtagccaacagcaagcctccacgatatgagttcctatggggtcccaaagcatatgctgaaaccaacaagatggccatcctaaagtttgtggccaaggtcaatgaaatctcccctagttacttcaaagatctttataaagaggctttaaaagatgatcaagggaagaatcagggcaatcataatgtcagctctgccattcatctcaaagatagggcattttcatttgtgatgtgtccacggcttgctttctccaatgttgaagtttaa

SEQ ID NO: 43 - DNA, 2751 bp, Homo sapiens
atagggagacttttctcaagagaattaaatacccaccgcatcagcccagtgggagccaggatgcatggccttttccttatgccagaaattatttggggagccacccaagctcagggcaatctgctgggctccccaatgcctgaagccagtggcagggtgcttcctgtctgccctggtgatggcaggggagtgagactggctattcacactcattacagttagtgcagtgggcagggtgtcactgactatgtaatcccaaggtctccaaacctcttgggaacagactaagcatccataggctagaggactggaaaactgggccaattgtccagttaccatggtgatgaggggcctctcacctcccagccccacaggggtgtggccttcagaggccagagtagctctgaaatagaatagagacagagattattcagaaggtagaaagtgggacagggagagagacttgcagcaaaagttaccactgatagttattctcttgtctgtgatcctgtcattcgggatgctgaggcaggaggattacacgtgcaagaactgtatagagaaaccctgtctcagtaaaggaaagagcagccataaactgagcccatgctgactgcccgcctccttgccctccactccctcccctaccactcctctttcctctcctcctcccaccctcttccctcttggttcccctcccgcttccctctgcaccttatccccacatgcatgcacccacccccgtctgcagggtaggcgacgctgtcctcggactggcctccatgttgctgctgcttgtgctgaagtgtatgcgggaacacatgcctcctccccatcctgagatgccccttgccgtgaagttcagccgtgggctggtgtggactgtcacaacagctcgcaatgccctggtggtctcctccgcggctctgattgcttacgccttcgaggtgacaggatcccatccctttgttctgactggaaagatcgccgaggggctccctccggtgcggatcccacccttctcagtgaccagggacaataagaccatctcgttctctgagatggtgcaggacatgggggccggactggctgtggtacctctgatggggctcctggagagcattgccgtggccaaatccttcgcgtctcagaataactaccgcattgatgctaaccaggaactactggccattggcctcaccaatgtgctgggctccctcgtctcctcttacccagtcactggcagctttgggcggacagctgtgaatgcccagacaggggtgtgtaccccggcaggaggcctggtgactggtgccctggtgctgctgtccctgaactacttgacctcactcttctcctatatccccaagtctgccctggctgccgtgatcatcacggctgtgaccccactctttgatgtcaagatcttcaggagtctctggcgcgttcagaggctggatctgctaccactgtgtgtgacgttcctgctgtccttctgggagatccagtacggtatcctggccggtagcctggtgtctttgctcattctcctgcactcggtagctaggcccaagactcaggtgtcagaaggacaaatttttgttcttcagccggccagcggcctgtacttccctgcaattgatgccctccgagaggcaataacgaaccgggcactggaagcatccccaccacgttccgcggttctggagtgcacgcatatcagcagtgtagactacaccgtgatcgtgggactcggtgagctcctggaggacttccagaagaaaggagtcgccctggcctttgttggcctacaggtgcccgtgctccgcacactgttggccgctgacctcaaggggttccgttacttcaccactctggaggaggctgagaaattcctgcagcaggaaccaggaactgagcccaacagcatccatgaagatgctgttccagagcaaaggagctccctgctcaagtctccctccggcccctgaagagcagatggtataggaagggtttctggaaggttctgtcaccatgacttggagtcacctgatagactcaccaacctggtgggacttaaaaggcactgcataggtggctctggggaacagcagggagccatgtatgatttccagggtgtcactttcctgctgtcccctaggtgtgagtatttgagggctgggctgactgaaaagtctttcagagagagagagagagagacagagacccagagacacacacatggcttctggcctggtctggcagggtaaggtgacactctccagatcccagattcttctttggaatcaggtcctactggagaaaaatcaaagagattgggcatctcggagatgtgtctgaccatgtcatgaagtctaatctgagctgaagaggtggccacagcatgccacacaaggtcaattctgtttaaaatcatgtgttttttaaatggaagtcactctggtgtttttgtaaagcaaaacaaaaacatttatgcttactttaaaaaaaaatccaagaactcatgcatcctgttcactattctcttaactgaggtcccctgcccggacaggtgtgtatccgtcagtgtgtgcacacggtgccgtctgatgtgaagacccgaatcactgtattaaggtgctttgtaaactgtcttcatgcagtcattaattgtgaattatttattgtgatgatcaatgacattaaatgcaagatttatttaccttattaaaatacaagatttatttattg

SEQ ID NO: 44 - DNA, 2862 bp, Homo sapiens
agcgattgattaatagctgggcgaggggacacactgactgttataataacactacaccagcaactcctggctccccaacagccggatcacaggcaggagagagtcagtgacggatagactttttttttctttaagaagccaacaacttggttgctagttttatttctgttaatttttttcttttttttggtgtgtgtggatgtgttgtggtggtcttttctaagtgtggagggcaaaaggagataccatcctaggctcagttcaacccctctccaaaaaaaacggcttctctggcgctccagctgcctgacagcctctacccggtggaagacctcgccgcctcgtcggtgaccatcttccccaatggtgaactgggaggcccctttgaccagatgaacggagtggcgggagatggcatgatcaacattgacatgactggagagaagagacccctggatctcccgtatccgagtagcttcgctcccatctctgcacctagaaaccagaccttcacctacatgggcaaattctccattgacccacagtaccctggtgccagctgctatccagaaggtatcatcaatattgtgagtgcgggcatcttgcaaggggtcacccctccagcttcaaccacagcctcctccagcgtcacctccgcctcccccaacccactggccacgggacccctgggtgtgtgtaccatgtcccagactcagcctgaactggaccacctctactctccgccaccacctcctcctccttattcgggctgtacaggagatctctaccaggatccttcagcattcttatcgccgccatccaccacttccacctcctctctggcctaccagccacctccttcctacccatcccccaagccagctatggacccaggtctcattcctatgatcccagactatcctggattttttccatctccgtgccagagagatccacacggtgctgctggcccagatcgaaagccgtttccctgtcctctggactccctgcgagtgccccctccactcacgccactctctaccatccgtaattttactctggggggtcccggtgctggagtcacgggaccaggagcaagtggaggtggtgagggacctcggctgcctggcagtgggtctgcagcagtgactgccaccccttataatccgcaccacctgccattgcggcccatcctgcgacctcgaaagtaccctaacaggcccagcaaaacgccagtgcacgaaaggccctatccctgcccagcagaaggttgtgataggaggttctcacgctctgatgagctgaccaggcacatccgaatccacacgggccacaagcccttccagtgtcggatctgcatgcgaaacttcagccgaagtgaccaccttactactcacatccgaacccacaccggggagaagccctttgcctgtgactattgtggccgcaagtttgccaggagtgacgaaaggaagcgccacaccaagatccaccttcggcagaaggaacggaagagcagtgctccctctgcacctccatctgcccagtcttcagcctctggtcctgggggctcgcaggccgggggcagcctgtgcggtaacagcgccattggaggaccactggcctcctgcacctctcgaaccaggacaccgtgagatgaagctctggctgacacaccagtttctccaggccccagaggccctctgtccgagctgccaacactaccacccttccctgttcctccccatgatcccgtgatctgggcaaaggaccttgatggagcccagctctgtcccaccttctcacggacggccttccgaaaacttaggccatttgaagggagttgactgtcactccaagaaatgggggagcaaaaagagggctgggtgagggcccctggcctacagggctgcgctctgaccctgactgagagatgtctgactatggtctgctagccctttccgttgaccctggatgccagttgttctgagactttttctacaataggttgggagttgctgattcctttgatcgaggacagcggaaaagactaaattaaagcaaaactgatgtggcactttaatggcttgggactgacttgggggtgggggtggggagttgtacagtgagcagagcctcatcctggctgctgcactctggccctagagcagtgaatggaggtttctctggccatctcaacccttaagcaatatgtcctagaaactcaagagaatggaagtgcaatgtcgggcaggacaaagcaatattggctcctttttttgttgtagttgaggaacaaagattattttttcagtgtatatccatttagattttgtgtatttttgatgtgcactgctctccgagttctgaaccttcgggaaaaaaatgtaaagcatttatgatctcttgaaatgagtcaaaggttaactaacttatttaaagggggacgtacataggatgcatgcagtgagtggtgttgcaagtgtcctctgtgccttgtgtgatgtgggcagtgttaacagggtctgcatgtgtacaggatgccttactatgggaacagaaaaatcactccttgggtttaagtatggctgtatatttctgcctattaatatttggaatttttttagaaagtatatttttgtatgctctgttttgtgacttaaaagtgttacctttgtagccaaatttcaggtgagaatgtgcttcaatgtcactgccgctgatttgtttggttattagctcttaatagttgtggagagagaaacaatctattctaacataaaaaaaccactaactggagttcagataatggatggcttattgactatggtgtaaataaatacttttcaacaataaaaaaaa

SEQ ID NO: 45 - DNA, 1956 bp, Homo sapiens
gagtctaccatgtgggagaagttctaaaccgccacataggttgcggttacccctcctaagtaagttgaactttgaaaacgtataattactcaccaaggctaagctgaccccatttctcaaccgactgagcctcttggaggaacgtttattacgtaatcactacccgtggaagaacagagaccgatctgagcctcggagggctcagacatcaggacatcagcatcccaagtggcttcgttcctttgaaaaaattgctgtagcacttagcagtctgcttaaaggagggagagaatcacactttgatctctgataagcgcaaattcaagtccttgagctcagcctttcaacatcactggcggttggcgtggtctccatcacagtggcggccattccgaactgttgtcgctgcagctctctccctttgatcccatcctgccaggtctcggagttcgcctgccatggctctacttactgcggcagcctggttcttgggaaccaaaaacccaccctgccttgtccttgccgcccggcatgccagtgcttcttccacgaacttgaaagacgtgctgaggaatctgatacccaaggagcaagccagaattaagaccttcagaaagaaacacgggaagacagttgtgggccagatcactgtggacatgatgtacggtggcatgagaggcatgaagggacttgtgtatgagacatcggttcttgatcctgatgagggcatccgtttccgaggctacagtatccctgagtgccagaaactgctgcctaaggctaagggtgggaaagaacccctgcctgagggcttattttggctgctggtaactggacagatgcccacagaggaacaggtatcttggctctcacaagaatgggtaaaaagggcagctctaccttctcatgtcgtcaccatgctggacaattttccgaccaaattgcaccccatgtctcagctcagtgcagccatcacagtcctcaacaatgaaagcaactttgcccgggcatatgcgcagggaatgaaccgaactaagtactgggagctcacctatgaagactgtatggacctgcttgccaagcttccgtgtgttgcagcaaagatctaccggaatctgtaccgggaagataggaatattgaggccattgactctaagctggactggtcccacaattttaccaacatgttaggctacacggaccctcagttcaccgagctcatgcgtttgtacctcaccatccacagtgaccatgagggtggtaatgtaagtgcccacaccagccatttggtgggcagtgccctttctgatccttacctgtcctttgcagcagccttgaatgggctggcggggcctctccatgggctagcaaatcaggaggtgcttgtctggctgacacagctacagaaggaagttggcgaagatgcatcagatgagaagttaaaaaactacatctggaacacactcaactcaggacgggttgtcccaggctatggccatgcagtactgaggaagactgaccctcgctattcctgtcagcgagagtttgctctgaaacatctgcctaaggatcccatgttcaagctggttggtcagctgtacaagattgtacccgatatcctcttagagcaagggaaggttaagaacccttggcccaacgtagacgctcacagtggggtgctgctccagtactacggcatgagggagatgaattactacacagtcctgttcggagtgtcgcgggcactgggcgttctgtcccagctcatctggagcagagccctaggcttccctctggaaaggcccaagtccatgagcacggatgctctgatgaagtttgtgaactctgagtcaggataaaatgggagacggggggtggggggggacgactgcccataagaaaacgaggataaaaatttattaaaaataaattctaatttttgttttgttccagggc

SEQ ID NO: 46 - DNA, 3909 bp, Homo sapiens
cagggagcgtcgcaagtttcccagccgcgtgcgaggacgcggtgcgggccagccagctagccccgcgtgcagcgctcaggaccgggctgactgcctaggagcatgggcagcgaccggagcgcactcggacgtccaggctgcactggcagctgcctcagcagccgagcttcgctgctcccgctgctactggtgcttctggactgtctgggccatggtacagcgtctaaagacgccgaagtgtacgccgcggagaactggctgcggctctatggctacctaccccagcccagccgccacatgtccaccatgcgctctgcccagatcctggcctccgcccttgccgagatgcagagtttctatgggatccctgtcacgggtgtgcttgatgaagagacgaaaacgtggatgaagcggccccgatgtggggttcctgatcagtttggggtacatgtgaaagccaacctgcgtcgacggcggaaacgttacaccctgacaggaaaggcatggaacaattaccacctgaccttcagcatccagaactacactgagaagctgggctggtacaactccatggaggcggtgcgcagggctttccaagtgtgggagcaggtcacaccattggtcttccaggaagtatcctatgatgacattcggctacgaaggcgagcggaggctgacatcatggtactctttgcctctggcttccatggcgacagctcaccgtttgatggcgtgggtggctttctggcccacgcttatttccccggccctggtctgggtggggacacccatttcgacgcagatgaaccctggaccttctccagcactgacctgcatggaatcagcctctttctggtggccgtgcatgagctgggccatgccctggggctagaacactcaagtaaccccagcgctattatggcacccttctaccagtggatggataccgacaacttccagctgcccgaagatgaccttcggggcatccagcagctgtatggctccccagatggtaagccacagcccacccggcctctccccactgtgaggccccggcggccaggacggccagatcaccagccacctcggcctccccagccaccacatccaggtgggaagccagaaaggccccccaaaccagggcccccaccccagccccgagccacagagaggcctgaccagtatggccccaacatctgcgatggcaacttcgacacagtggctgtgctccgcggagagatgtttgtgttcaagggccgctggttctggcgagtcaggcacaaccgcgttctagacaactaccccatgccaattggccacttctggcgcggtctgccggggaacatcagtgctgcctacgagcgccaggatggacattttgtcttcttcaaaggtaaccgctactggcttttccgagaagccaatctggagcccggctacccacagccgctgagcagctatggcacagacatcccctatgaccgcattgacacagccatctggtgggagcccacaggtcacaccttcttctttcaagcagacaggtactggcgcttcaacgaggagacacagcatggagaccctggctaccccaagcccatcagtgtctggcaggggatccccacctctcccaaaggggccttcctcagcaacgatgcagcctacacctacttctacaagggcaccaagtactggaaattcaacaacgaacgcctacggatggaacccggccaccccaaatccatcctgcgggacttcatgggctgccaggagcacgtggagccccgatcgcgatggcccgatgtggctcgtccacccttcaaccccaacgggggtgccgagcctgaggcagatggtgacagcaaggaagagaacgcgggtgacaaggatgagggcagccgcgtggtggtgcagatggaggaggtggtccggacagtgaacgtggtgatggtgttggtccccttgctgctgttgctctgtattctgggcctggcctttgctctggtgcagatgcagcgcaagggcgcccctcgcatgctgctctactgcaagcgctcactgcaggagtgggtgtgatcaggtagcacccgcagcacccggtgctcaggtctgcagggacaggagacgtgtggatcccagatgctccctcggcgcggcaggatgccctcctagccacacacatccccctgccagccttgccccccacccccgtcctcctttatttatgcccaggtgcctcttctttttggcaccctctctcagcctttggttccgctttcctgactcgggccaggagatgctttgagatctcccggagtgtagtaccgccccaggcaggaacgaagggacagaggctcacacctctggaagatggcagagggctgggaagcttctctggctctggcttttcagcctgaacctctcctcccacgacaagaactgtgtttgccttgaaggagcccacctcgggctctgccctcctgaatttgccttagggagagtatatgctcccccccctggggggggcaccctgataaccttgagacttggttccttcttcttaggctcctaagaagcccagaagcctaaaagccctcccccatcctagcattctgcagctcctacttgccccgggcctgcagcttctttcctctaggaaggggccctggacatgtctcacctaggaccaaagggagcctgccaagctcccctcccaatgctagtgactagctcctccttccctaggctgcccatattctctttcagtgccccataggtctccggaggctgggcactccccgcccgcccccacccccagcttctgcagtccttgtcccaactcttaatgacatggggcagacacacccactagggtaccagaggtccctgggcagtgtgtgtgattgtggatgctcagcaggtcacagaccaagcttatgggcttcctcccttccagattcctgtccccacacaacactactaggctcaggccagcagctcaccccacacagagtctgtggaacccccaaaggaatccactaaggcctgggaagtctgtcctaagggtgaaaggggccctcccttcctcttctcatcctggagtcagcaggaggcttctgtgggaaatggtactgtacagctgtggctctgtctcctctgcctccctaaagccttgagcagtaaagggctgccagccaggatcctctgggtcaccgccgctttccaagtggtggattcttatagcctggcactagaggtgacagatcggaggggcatgtcctgcagcactctagatgtcccctctgcagaacctgagagtcagcaaggggtggggcgtccacccccccactccccatcgtttagttctgtagataatcagcactgattaaatgtccagccagcgagcgtggctgcacttgtacgctcagacccaagcatgccgcaaggaagacctgcttggcagggtcggggccaagcgaggatgccgaggaataccctcctcccagtttggtaggacctttgggatggagtccaggaaggtcagggaagagtggggggggggggcagtaggagtctggttgggctgaggtttactgggtttacttaacatttggtagtgataagaggagaggacagcccagcttcccaaatgactccacatctggcggtctggaagaaccagacaaggccgcccaatcgtcccacccacctggattggattttgggcagcctgagaggtggcttgaacaagaactcagggatgggtctggagagaattgtgtaacggggccactttacctccagaatcttccctccatgttcagagaataaaatgcactggaacctgcaaaaaaaaaaaaaaaaaaaaaaaaaaaaaaaaaaaaaaaaaaaaaaaaaaaaaaaaaaaaa

SEQ ID NO: 47 - DNA, 4646 bp, Homo sapiens
gcggcgcccccactttccaacccctgcggcgagagcggatgcagatggtccgggagacaggcctctctttctgatgaaaagaggagggaaggatggactacactcaccaacctgccctaatcccatgcggacaagacaagtatatgcccaagagtgaacttctcctacatctgaagacctacaacttgtactatgaaggtcaaaacctgcaactccgacaccgagaggaggaagacgagttcattgtggaggggttactgaacatctcctgggggctgcgcaggcccattcggctgcagatgcaggatgaccacgaacgcatccgtcctcctccatcctcctcctcctggcactccggctgtaacctgggcgcacagggcaccacactgaagcccctcaccatgcccactgttcagatctcagaagtggatatgccagttgaaggcctcgagacacacagcccaacagattccagaggcctgaagcctgtgcaggaggacaccccacagttgatgcgcacacgcagcgatgttggggtgcgtcgccgtggcaacgtgaggacatccagtgaccagcgacggatcagacgccatcgcttctccatcaatggccatttctataaccacaagacatcagtgttcacaccagcctatggctctgtcaccaacgtccgcatcaacagcaccatgaccactccgcaggtcctgaagttgctgctcaacaagtttaagattgagaactcggcagaggagtttgcgctgtacgtggtccacaccagcggtgaaaagcagaggctgaagagctccgactaccctctgatcgcccgcatcctccagggcccttgtgaacagatctccaaagttttcctgatggagaaggaccaggtggaggaggtcacctatgacgtggcccagtatataaagttcgagatgccagtccttaaaagcttcattcagaaactccaggaggaggaagaccgggaggtggagaagctgatgcgcaagtacactgtgctccggctaatgattcggcaaaggctcgaggagatagcagagacccccgagactatctgagccacaggaatgaagacagtccagacactccaggggccgaggctgacctgagaagacccactggaagctaggcatcctacagaaacgttttcacacgatccttgcagccttccaaaagccagcactgctccctacagtaggacacagaagcagcagacagctccccacctttggacccctgatcttctttgtctcattctcaaggacttttgtcctaaggaggacccacaatgtgtatgcttgcatacctgagtgtgtgtatgtgtatgtgcacgcatacgcaagtgtgcatatgtgtacttaccatgtccacgtgcctctctgatgttcttacatttggttaaattcccagcagccagcaggtgtcacaaggcaggagggcgcacgtgctgctcctgtgtgggaagaacagggaagtgactgaggcttttggcttcctccagtgccaacggcagccgcacggatctgccacgcttgacagggaagaggaggcagcaggctcaccgtttggaatgtctgaaggtttacagcagagattcttctcatttgccaaggggtggccccatgcccttggggcaagtccaacgaaatggaaaaagcaagatcacctcagggcggatgcaatgaatctatgagtttttgcactggaaaggttatcaatgtagaaagcacaattaatgtccccgtcctggatggaaacctagaaagaatggattctgccttcgttcttggctttgtggaggcttctgggtgtttctgctctccagctgccacttcaattcaccctgacctgctcatgtgcctgggccaggtgtgtgctgggataaacggcactcgaaagtttttaagccaccagctgagatgagaggtacctttaagcctagcccaccatcacatcggtgagagtatctcccacgtggaggtggagcagtgtgtttgaagtcgcatgtctttctttctggtctctgcacacttgcaagctccttcacctgatacacggagatggattccaagaatggagacctgtcctgttgctcagtcaacacagggagtcaggaagctctttggggtgttccatgagggtgggcagagaggacggtgcttgcttagcctttgagaagccatgctcccgattttcagaaccagcctcattggcagaaagggcagggatgatcctgtaacccctccattcgggatgctgtcatgtagctggtcacctgtgtggcttgtaccatttgagtttggacggatggatgggcttgctgctggtctatgaaactcagccaagcaaggggagcagcccataactcaagcatctctgagttttgctacaacagggtgttcttgtctacctgctctcaagaaagctctagaagcacagcagcggagagactgaggggccccgaccagtgcgccttgtaaggaaggtggacctctgcagcagatctgttctagtgtaggaatgttactgtcctttgcccttctcagaggctcacagctgtgatatcattgtgggctttcaaacagatggctcatggccagccattttgctgggtatcacttagcaaccttgctcttcttctaggacagcacaatccatcccatgagcagtgggatggaaacctgtgggtctgagtctttatcttagactcaaaagacagcaaagcacagaactgagcacagatactgagtgtatctttatgagtacacacttcagtaaactctgagctcagtgggcagctggggtgggggttcctttatgacagacaaagaacagctttgtgaatgcaaatacttgagaaaatttggtttggataacttctcttcccgttatctttatcttgaatgttcagagcctatgttggtccacaattctcccttgggagcctgggggctttccatggagcacacgaactgggaagttatttctgatttatgaaagctcattgtgtaagtgtcaatatgtgactgttgctgggcatcgggttgccaggctttccaagatggtggtgccaagatagacagcttttaaagactggggttcagaaagggagttggggtacatgttcactaatccatcttggcacattccctagagatcatgccttatgtccaaagaactgggcaggagttttaggaaattggctgggaatgagactcagtggtggaacattttcttagtgtgtgtgaaaagccgacttcaatctctagaaacacacacacacacacacacacacacacacacacacacacacacacacacacacacatgcacgcatgcacgcacgcacgcagcagtggtgtagctcagtgatagagggattgcttagagtgtcagaggctggttcccgacgcctgcaccaagcaccacactaccccctgcactttggaagcctttgacatagccgcagcagaacagacctctacagtgacgactggattttctcactggtctcacttcacagctttgaactccaagactgattagtgtgggcttatgtaattgggtgcaatggacctgaaatcctgagaaataaaagactaacgcataaggcggcggtgggggtgctaatccagcatgtgaaaagtggttcggctgcctctggactcttcccctcccccacccccacccccacaaaggcttattttacaaggtgatcaagtgccgagaacaccagcctccgcctgagcgaggtggtgaggccaagcacaggttgactttctcggtgaccatgttagcggggagctgatgcctcacagtgggtgggggtgttagttggaaaggggcccaaagcacaaacacccgtcatgcctgtgctactggtgaagggcacacagctaacaccatggacaaacgcagctttgtaacgtggaaagtttggggttagggatacatttccaagaaagaaatatgtccagtgaccaaaggagttttaatgtcccacaggagtcagctttgccctggtgagggaggaggtaggaggctgccctagctcacccccacagcactgcctatcttaaagcatatcacctaggttgttttctggaaagcctggcttccgccggatgagagagcatctatgaacctgatgtgacatcccttccttcagacataattgacagtggttgccattccttggggtacaccttcctcccttcctcctgctcccattaaactgagcacagaggtgcttgaactgtgaactttggagaagcctcagtctccaggttgctggattctggggttgctgggttctggggttactactagtgtttcttcgggttttttcttagttgagtggagtcttttttttttttttttcttcatcataacttgcctgttaggaatggtacatgacttggcttaactgttggttatgaactgaaaatcggatgtgcttaggaatcttaacatgattggatgggaccttgatggggagccatggttttgtagccttttattttttaattattattttttttgtaatgtatccatcttagaccttatttaaataaagcacttttggaagcgaaaaaaaaaaaaaaaaaaaaaaaaaaaaaaaaaaaaaaaaa

SEQ ID NO: 48 - DNA, 4842 bp, Homo sapiens
cgacgttgtgcttctaacagctctccgaggtccctgccggaagtgtaggaagaagcagacagatttgaacatctctgtttccagcttctctgatcatcatgatgaagattagacacaaaaacaaaaaaccaggtaaaggttccaaaggctgtaagaagcctgcaaggcaaaatgggaagaaagtaacctccagaccatcatctgctccccagattgttcatggcaatgaccatgccagtcgcgaggccgaattaaagaagaaaagggtcgaggagatgagggagaagcagcaagttgcccgggagcaagagagacagagacacaggaccatggagagctattgtcaggatgtcctgaaacgtcagcaggaatttgaacaaaaggaggaagttttgcaggaattaaacatgtttcctcagttggatgatgaggccacaaggaaggcctattacaaggaattccggaaggtggtagagtactctgatgtgattctggaagtcctagatgccagagacccattgggctgccgctgtttccagatggaggagactgtccttcgtgcagaaggcaacaagaagctggtcttggtcttaaataagatagatctcgttcccaaggagattgtggaaaagtggctggaataccttctcaatgaactgccaactgtggctttcaaggccagcacccagcatcatcaggtcaaaaacttgactcgttgtaaagttccagtggaccaggcctctgagtcgcttttgaaaagcagagcctgctttggagccgaaaatctcatgagggtcctggggaactattgtcgcctgggggaagtgcgtggccacattcgtgtgggtgttgtaggccttcccaatgtggggaagagcagtctgatcaatagcctgaagcgcagccgtgcttgtagtgtgggagctgttcctggtgtcacaaaattcatgcaggaggtctacctagacaagtttatcaggcttctggatgcaccaggcattgtcccaggacccaattcagaggtgggcaccatcctgcgtaattgcatccatgtgcagaagctggcagaccctgtgaccccggtggagaccatccttcagcgctgcaacctggaggagatttccagctactatggtgtatctggattccagacgactgagcactttctgactgcagtggcccatcgcttgggaaagaagaagaagggaggtgtatatagtcaggaacaggctgccaaagctgtgctggctgactgggtgagtgggaagatcagcttctatacactaccaccgcccactcacactctgcccacccatctcagtgctgagattgttaaggagatgactgaggtctttgatatagaagatactgagcacgccaatgaagacaccatggaatgcttagctgtgggagaatccgatgagctgttgggtgacatggacccacaagaaatggaggtcaggtggctccattctccactggtgaaaatagcagatgctattgaaaatagaagcaccgtgtataagattggaaatctcactgggtattgtaccaaaccaaaccgtaatcagatggggtggcctaaacgcaatgtggaccaccattgcccccaaaataaccgtgtagtagaggtcagttctgtggaccgccgcccgatgttgcagaggatcctggagacagacccacttcagcaaggccaggctctggcatctgccttgaagaataagaaaaaattgcagaagcgttcagataaaatcgccactaagttgtctgactccatgatgtccatgcttgacctctctggcaactccgatgactgtgcaggtgactgagcagctgaccttccccctcatactgcaagtactgcttcccgtgcgatgggagagtccagatgcctttccattctccctgatgtacttaataggagaacacctccatgtcatgtatcttctgctgccgtgccctccattcttatcaaaggtctggagctgccaaaccctactaccctctacttaatccttcctgtctttgaaaatgaccttgtgtttgttttgtggtgccagggattgaacataataggttctcacaaatgttagacaaccattctattaccaagctgtagtattaccatcattaccatccatcattatcattggggagtctgcttactcaacctcccaagtactgggattacaggcttttgccaccattcccagctcccactgtgttttctaaggtaaataaaatcagcagactcttggcatggtggcaacatatctataaccctgacacttgggagactggagctaggaggactgtaagttatgggccagcctgaactatctggcaagaccctttctcaacaaaacagcccaaaacaaataagcagagtccagctgggcatacacctttaatcctagcactctggaggcagaggcagatgaatctcgtgagtttcaggccagcctggtctaccgagcaagttccaggatagccagggctgcatagagaaactccgtctcgaaaagtgaacaataaaacaacaaaaagaatttgtaattatttaaaaagcaaataagcagagtccaagtgctgacaatgtaccttggactctaaattcctgtgtcgtaccctgtgagttaggctgataagggttaaccttttggacaggtaaatgtcctaaaggctagactgctgagaaacctggtagcagtggcgggggaggtggcggttgtccctggatgggtgatccttttcatttttatcaccctacaagatctcagtgccaaggctcttgcctgtgtattttccaaatcaatagctagctcctcatctgggtagtgtaggagtagagaaggatagtgtgagtctcctgtctctggaagtgtctggggatagtgtagaggatgacgattctagaaagggtatacctatcatagtaagttggaaagccattggctgcaggatgttcaggaaatgtagaccgtacctgttttctggacggttaggggctgagcacaaactactaaacaatgagaaacagttaccacctttaactttcactatatccgtatcaggaaggggtaaggctcagaaagccgacagaatgtgctcatgtcaacagtaagtggtggtgtgctagatgtcaaacttgagtctaatttaaaccttatgctagccttttggtttgcattattttgccttttcaacaagggcacaccactctggtattttctgttgtcatatatttaaaatatattttctctactctgttttttatttttatttatttatttattttttgggtttttcgagacagggtttctctgtatagtcttggctgtcctggaactcactttgtagaccaggctggcctcgaactcagaaatccgcctacctctgcctcccaagtgctgggattaaaggcgtgcgccaccaccgcccggctctgttttttaaaaaatggtagtatttattgaaaaactatgctttctaagtctctttaaatgatgtccaacttaattcactgatctcatcttaagttttgctagacaataatatattagaaataaaagtatctagattcttgggaacataatcatgtcaaaataaaatagaactaaaaccaggtgggtggctcacaccagtgctccaagcactaagaatgctgaagcaggaagatgtctgaagctgagtccagcctgggctgcagagtaagaggcagtctcagggcgaaacaaaataatgtgggactttagatattctaactctaatgtatctcaaggttgggggtagcatgcgcacgcacgcacagagagagagtttcccccatataacccagtgtggtcctgaactttttaggccccccccccattagctttccaagtgctgggattgcaggtgtttgctttcatgcctggatatttctttgttcttaaaacaatttcttgggtctttactctccccttaaaaatcatttttatacaataaatctcatccttcatggttctatggttctgtaagtttagacacctttttttttttttttttttttccgagacagggtttctctgtgtaatcctggctgtcctggaactcaccaggctggcctcgaactcagaaatccgcctgcctctgcctcccaagtgctgggattaaaggcgtgcgccaccacgcccggttatttagacaccttttaacaggggtggcctgccatcataatcaagataagagaattttgtggcatttccccaaacttattgtgtcctcttgtagtatagtcctgcccccatcctcaaatcctagcaggcacagacctctttcctgttccttaatataagtgtcgtaagtcacacgggggcttattttatggcattaagcttttagctttttccttttagatcttacattgaagttagatgcagttgtataagctcggataaatggataaagccgagtaatcattaatacaggacagaagagttctattagcctctcctttaccattcctggtaatttatagtcaaaccttgttctctccctgacagtcacggacttgtgctttcactttatggcttgtcttttgaaaagagttgtaagtggaatttaatagtatgtaacttttgagattaatgtgttataaattatagacttgagatccatatcactggatgtatcaatagtttgatttctgtttcatatgctattgtgtggatgtcttattgcttattcaaccctaagttggattacattctttacagttgaggaggaagttataaataaagctgctaaaaatcaaaaaaaaaaaaaaaaaaaaaaaaaaaaaaaaaaaaaaaaaaaaaa

SEQ ID NO: 49 - DNA, 6256 bp, Homo sapiens
ccacgcgtccgctcctttaccaagatggcgtcttgttcctgtttcgccacagttcctaccttatgagcttggattcctcacgctaataaaagaggagcagtaaaagctggcaggctgacagaggcaacctcaggacggactttctggctactgaccattttgctgtgtcttactaggatcgtgtgtggacgtgagatcaaccatgagctccgttgcagttttgacccaagagagttttgctgaacatcgaagtgggctggttccacaacaaatcaaagttgccaccttaaattcagaagaggaaaatgaccctccaacctacaaggatgccttccctccacttcctgagaaagcagcttgcttggagagtgcccaagaacctgccggagcctggagtaacaagatccggcccatcaaggcctctgtcatcactcaggtgtttcatgtacccctggaagagagaaaatataaggacatgaaccagtttggagagggtgaacaagcaaaaatctgccttgagatcatgcagaggactggtgctcacctggaactctctctggctaaagatcaaggtctctccatcatggtctcagggaagctggatgccgtcatgaaagcccgcaaagacattgttgctagactacagacacaggcctcagcaactgttcccattcctaaagagcaccatcgctttgttattggcaaaaacggagagaaactgcaagacttagaactaaaaactgcaaccaaaatccagatcccacgcccagatgaccccagtaatcagatcaagatcactggcaccaaagaaggcatcgagaaagctcgccatgaagtcctcctaatctctgctgagcaggacaagcgtgctgtagagaggctagaagtggagaaggctttccaccccttcatcgctgggccgtacaacagacttgtgggtgaaatcatgcaggagacagggacgcgaatcaacatccctccacccagtgttaaccgtacagaaatcgtcttcactggagagaaggagcagctggctcaggctgtggctcgcattaagaagatttatgaggaaaagaagaagaagactacaaccattgcggtagaggtgaagaagtctcagcacaagtatgttattgggccaaagggcaattcactgcaagaaatcctggaaagaactggagtttctgttgagatcccaccctccgacagcatctctgagactgtgatacttagaggcgaacctgaaaagttgggacaggcgttgactgaagtctatgccaaggccaacagttttacagtctcctcagtctctgccccttcctggcttcatcgtttcatcattggtaagaaagggcagaacctggccaaaatcactcagcaaatgccaaaggttcacattgagttcacagaaggtgaggacaagatcaccctggagggccccacagaggatgtgaatgtagcccaagaacagattgaaggaatggtcaaagacctgattaataggatggactatgtagagatcaacattgaccataaattccacagacacctcattggaaagagcggagccaacatcaacagaatcaaagaccagtacaaagtgtctgtgcgcatcccccctgacagtgaaaagagtaacctgatccgaattgagggagacccacagggtgttcagcaggccaagcgggagctgctggagcttgcttctcgcatggaaaatgagcgcaccaaggatctaatcatcgagcaaagatttcatcgcacaatcattgggcagaaaggtgaacggattcgagaaattcgtgacaaattcccagaggttatcatcaactttccagacccagcacaaaaaagtgatattgtacaactcagaggccccaagaatgaggtggagaagtgcactaagtatatgcagaagatggtcgcagacctggtggaaaatagctattcaatttctgtaccaatcttcaaacagtttcacaagaatatcatcgggaaaggaggcgcaaacattaaaaagatccgtgaagagagcaacaccaagattgaccttcctgcagagaacagcaactctgagaccattattatcactggcaagcgagccaactgtgaagctgcccgcagccggattctgtctatccagaaagatctggccaacatagctgaagtggaggtctcaattcctgccaaactacacaactctctcattggcacaaagggccgtctgatccgctccatcatggaggaatgtggtggcgtccatattcactttcctgtggaaggttctggaagcgacactgttgtcatcaggggtccttcctcagatgtagaaaaggccaagaagcagctcctgcacctggctgaggagaagcaaaccaagagttttactgtggacatccgagccaagccagaataccacaaattcctcattggcaaagggggaggtaaaatccgaaaggtgcgtgacagtactggagcccgcatcatatttccggctgctgaagataaggaccaggacctgatcacaatcataggaaaggaggatgctgtccgtgaggcccaaaaggagctggaagcattgatccagaacctggagaatgtggttgaagactatatgctggtggaccccaaacaccaccgccattttgtcatccgaagaggacaagttttacgagaaattgctgaagagtatggcggggtgatggtcagcttcccacgctctggcacgcagagtgataaagtcactctcaagggtgccaaggactgtgtggaggcagccaagaaacgcattcaggaaatcattgaggatctggaagctcaagtgacagtggaatgtgctataccccagaagtttcaccgatcggtcatgggtcccaaaggttcaagaatccagcagatcactcgggactacaatgttcaaatcaagttcccagatagagaggaaaatccagttcacagtgtagagccttccatccaggagaatggagatgaggctggagagggcagagaggccaaggagaccgaccctggctctcccaggaggtgtgacatcataatcatctctggccggaaagagaaatgcgaggctgccaaggaagccctggaggcactggttccagtcaccatcgaggtagaggtaccctttgatcttcaccgctacatcattgggcaaaaaggaagtggaatccgcaagatgatggatgagtttgaggtgaacatccatgtaccagcccccgagctgcagtcagacaccatcgccatcactggccttgcagcaaatttggacagggctaaggcggggctactggatcgagtaaaggagctgcaggctgagcaggaagatcgggctttaaggagttttaagctgagtgtcactgtagaccccaaataccatcccaaaattatcgggagaaaaggagcagtgatcacccagatccgcttggaacatgaagtgaacatccaatttcctgataaggatgatgggaaccagccccaggaccaaatcaccatcacagggtatgagaagaacactgaagctgcccgtgatgctatcctgaaaattgtgggtgagcttgagcagatggtttctgaggatgttccactggatcaccgtgtgcacgctcgaatcattggtgcccggggcaaagccatccggaaaataatggatgaattcaaggtggacattcgcttcccacagagtggagcgccagaccccaattgcgtcactgtgacaggactcccagagaatgtggaggaagctatcgaccatatcctcaacctagaagaggaatatctggcagatgtggtggacagtgaggcactgcaagtttacatgaagcccccagcacatgaagagtccagggcaccatccaaaggctttgtggtgcgagatgctccctggacctccaacagcagtgagaaggctcctgatatgagcagctctgaagaatttcccagctttggggctcaagtggcccccaagaccctcccttggggccccaaacgataatgatcaaaaagaaacctttccagcctgctgcccaaacccaaccacacaatggtttgtctcaatctgacccagcagctggaccctcgtaaattgttgatgctctttcccctccccgaggtctcgcagcaaagcctggtgggctggccttgtgtggctgcttctccaggcctggtcagcacccagcaggctcagggtctgctcctgatgctgtgctctgggacaggcacgccactgtgtgaaacactaagcaaggtaatcgagcatttcgtgatcacagactccagcttcctggtccacccagcatgtagtcagcactctgaccttcacaccagagctccacagcggctaggagttgacttcctgtgtcatgacctcaggaaataaatttccttgactttataaaagccaaaacgtttgccctcttcctttcccacctccctcctgcccaccacctgtcgcccagaccttcctctgttgaaagcagtcagcttcctttagctgtcaaaatctgcttagcacaggcttcaggataggaggaaggcatggtatcggacagcaagaggtcagatgtcctggtgatagagtgggctgagaagtagagggtggcctgggtcctcagatagtgctgaccgtgcccactttgggttccgaaagcatcaggggcttctattgcagctttagcctcatgatcctatgtatcttagaggcacagctgtgtgtattccacttgggttccctgtgactatcacagtggactagggtggcttgagactttgggggaaaggggaggtagagactggcttttcttttggtgatggggagggggctcaaccctaagtgacagctcagctttcccacgacctttagcctctgcctgccttgttcagttgccttctttctacctcagccagtgtggtagtactctcatgtgggttagtgccacccaaggctcttcttgtgatgtagacatttctggtgattctgggcttgccattgagttgagggtggaggccagtgctttccctcagtccacatgattcagggactgggccagagctgggtgttccttcccagccagcctgaccatgcaggctctggcaagcataacctggatgctttatgcagtcctaaggaacaggcttaaggggcaggtctcgtcgatagaggtggtcatttagggtggattcacagggaaggcagaatgaaaacaacccaaagcagggagcacagctatctgaaggagatcttttcctcaggtacaagaggtaatttttggaccactggcctccagctagcttggctgtaggccaaccctcaccaagaaaagtggtcccgagcagacaaacccctgtcaggctgtggaaactgaggacagaccttttatctccaggtcatctgatcaactagcaacatgtcccctcccagatacttgggttactagaaaagccaacttgtggcgatctcatggccttccctgggaactgtcacttctactatgatacctttccccctcaagtctttgactcccccatttaataactcaggcttggctacctgtagactctaatcctaagtggatccagaattgctttcccaagtctaacttcagttccctttggatggaaccagtcccacatgtacccacttagaagaacgatactgagatgggaaaagaacatctactttgttcagtaggctggcagcttaagccaggtgggtgagacggggagagcgcttccctgcctttccctggagccgtctcttctcccatctgcatgagactggggacaccttgcttccctccttcccttccagccccctcttcatcttcattgtcatcctacaacatccaatctcccctcactgaaggtaaagtttaaattatcaccaggattttgcacatctccttttattgctaagtgctgtagatgtaagccctatcctaagctgttcagggtctcactccttttctctgattgttaataaggactatgaaattgactgtacccaggtcacctctctgcagcctccaagatgttgaggagcctagggcaggggtgggagtgtcttttcctccttgttgaatgtcttccatgtcctatgtttccatagctacatccatcccttggccttaactttggaatttggagattatatgcaaacatgtgtaaaggctcatgaatatggatgacactggaattttataaattctaaaataaaacccaaaaccagaaaaaaaaaaaaaaa

SEQ ID NO: 50 - DNA, 436 bp, Homo sapiens
ctcaaagaggtaagctccacccttaagaagcttcacaggccccattagaggacccaccccatagactatggccttcatgtatacgaaaggcttcgcttccttcacagagaaaggaaataatgaacagaaattatttctgagagtactgggcacattaccaaccctgaggcaagaagagaaaatcaaagggccagaatcagtgcagtgatgtggcacccagctgtacagagagtggagttttctggctatgtcctggaaaacactatattggaagcaaatgacaaagtgccctgtttgagtcagttttaccagaacagtcctgtgaatgtgtaagtgcatactgccagaagtgtgtatccagtttgtgtacaaagaactgttttaatatatatacacatacatacacagactaggggctaatgtcttttattaaatt

SEQ ID NO: 51 - DNA, 3733 bp, Homo sapiens
ctgcctcgcagccgtcgggatggaggtgagacggtagccgcgacgcccgccctcgcgcacccctgcaccacagcggcctgcagagctccccgccccccgcccccgcggcagcgggccttgccgtcgagtgacagcggcctgggggggcagggggggcgggggcggccagaccagcgatgccggcgggcatgacgaagcatggctcgcgctccaccagctcgctgccgcccgagcccatggagatcgtgcgcagcaaggcgtgctcgcgccgggtgcgcctcaacgtcgggggcctggcgcacgaggtgctgtggcgcactctggaccgcctgcctcgcacgcggctgggcaagctccgggactgcaacacgcacgactctctgctccaggtgtgcgacgactacagcctcgaggacaacgagtacttcttcgaccgccaccctggcgccttcacctctattctcaatttctaccgcaccggccggctgcacatgatggaggagatgtgcgcgctgagcttcagccaggagctggactactggggcatcgatgagatctacctggagtcctgctgccaggcccgctaccaccaaaagaaggagcagatgaacgaggagctgaagcgggaggctgagacgctgcgggagcgggagggcgaggagttcgacaacacgtgctgtgctgagaagaggaagaaactttgggacctgctggagaagcccaactcatcggtggccgccaagatcctggccatcatctccatcatgttcattgtcctctccaccattgccctgtcactcaacacactgcctgagctacagagcctggacgaattcggccagagcacggacaacccgcagctggcacacgtggaggctgtgtgcatcgcgtggttcaccatggagtacttgctgaggttcctgtcctcgcccaagaaatggaagttctttaagggccccctcaacgccattgacttactggccatcctgccctactacgtcaccatcttcctcacagaatccaacaagagcgtgctgcagttccagaatgtgcgccgtgtggtccagatcttccgcatcatgcgcatcctgcgcatcctgaagttggcccgccactccaccggtctgcagtccttgggcttcacgctgcgcaggagctacaacgagctgggcttgctcatcctcttcctcgccatgggcatcatgatcttctccagcctggtcttctttgccgagaaggatgaggatgacaccaagttcaaaagcatccccgcctctttctggtgggctaccatcaccatgacgaccgttggttacggagacatctaccctaagactctcctggggaaaatcgtggggggcctctgttgcattgccggtgtcctggtgattgccctccccattccaattatcgtcaataacttctccgagttctacaaggagcagaagcgccaggagaaagccatcaagcggagagaggctctggagagagccaagaggaacggcagcatcgtgtccatgaacatgaaggatgccttcgcccggagcatcgagatgatggacatcgtggtggagaaaaatggagagggcgtggctaagaaggacaaggtgcaggataaccacctgtctcccaacaagtggaaatggaccaagagggcgctctccgagaccagctcgagcaagtcctttgaaaccaaggagcagggatctcctgagaaggccaggtcctcgtctagtccgcagcacttgaacgtccagcagctgcaagacatgtacagcaagatggccaagacccagtctcaacccatcctcaacaccaaggagatggcgccgcagagccagccgcaggaagaactggagatgggcagcatgcccagccccgtggcccctctgcccacgcgcacagagggcgtcatcgacatgcgcagcatgtctagcatcgacagcttcatcagctgcgccacggacttccctgaagccaccagattctcccacagtcctctggcatccctctccggcaagtctgggggcagcacagccccggaggtgggctggcggggggctctgggtgccagcggcgggagactcatggagaccaaccccatccccgaggccagccgctctggtttcttcgtggagagcccccggagttccatgaagacccacaaccccatgaagctgcgagcgctcaaggttaacttcctggagggcgatcccaccccgctgctaccggctctgggcttgtatcacgatcctcttaggaacagaggaggcgcagcggctgcagtcgccggactggagtgtgcctccctcttagacaagcccgtgctgagcccggagtcctccatctacaccacagcaagtgccaggacgccccctcgctccccagagaaacacacagcaatagcgttcaacttcgaggcgggggtccaccagtacatagacaccgacactgatgacgagggtcagctgctctacagcgtggactccagtcctcccaagagtctccacgggagtaccagccccaagttcagccttggagctagaacagaaaagaaccacttcgagagctccccgttgcccacctcccctaagttcttaaggccgaactgtgtctacgcctcagaagggttacctgggaaaggtcccggggcccaagagaaatgcaagctggagaaccacacctcccccgatgtgcacatgctgcctgggggaggagcacacgggagcactcgggatcagagtatctgagctccctaccacagagggtatttgcaggagacatcgaggaggagggatgcttggcctgcctgccgctgagttctgcgtgaactcggagacccaaggaccccacacacacacaattttccccacccccgcagagccccagcgacaggagagaccctgagagccacgacaagtctgtggacaccaactaggatggggaccacacctaacagcttcttccttagatggcaggagcagagctgacagccacatactcctagtctcctttagaggctctgtctgcctctgagtgcgtcatcgccatctctcatggcgtcagctgaggcaggtgttggagcaaggggttgctagtgagagagtgggctggccaagcagtagtgggtgtcccccctcttgttagaggggtacagaggacacttgatcctgagggagtctccacttcatcagccacactcctagggcataacgcttaaacaagcagcggctcaaatgattcagtattctgtggtatctggggctttagtctgagaagctgtcaggaggagctgacaatttaaactcaatcctttctgctcacctggcaatccagaaagggttccaacagttggcacgcagccaaggggatataagccagggctgtcacaggaatgcgggtcccaaacagggctagcccggactgaaaagataagcgtcaaatatgcaaacgagatagattattcaaaaagactgtgtgtgactgaaaaacaacattaaaaaaataataaatttcattttctacatgaaaaaaaaaagaaaaaagaaaagaaaaatccttcactgaactgccctgtgaagaattgtgactactttctgctgtggtgtggtagggaacacacctactatgagaggaagttctctctctctttttttttctttctttttttgtttcctgtagtattcaagctaaaaaataataataataataagttaataaaaaaaaaaaaaaa

SEQ ID NO: 52 - DNA, 7048 bp, Homo sapiens
ggtccccgcgccgcggccgcgccaatccccgcgccgccgccgccgtcgccgctgtccctgccaccgccgatctagcagcagctctaaccccggctccggctacccagcgcacgtccgcagcccaggtcattggcgagcctaagctcagccccctcccggagcccgagcccggagcccggcgcccggagccagccccagccccgcggccgccgcggctgcggggagagggcgggggcgatgctgccggagccgccgccgctgccgccgcctcgatgagagccgcgccgcaccgcgcatagccgcgcaggctgacaggcagaaagactgacttccctctcccgggcatcctccctgggctgccgggaggcggcggcggaggaggaggaggagggaaaaggggagaaggcggagagcaggaacgagaggaggtggacctggatccgtttctccgggccaggactcgagcggccccagctaccgctacccgccggcgccgtcccctatccatcaaccctcctgcacccatccgcgaccctgggctctctgcgcgtcgggccggggctggagcggcgcggccgcagactgtctggcttccccactatgcccatgctttgctaagtgctggtggccatcgaagtttttgcatcctggggacgaatcctgagctgaccagagacggacggcgcaaggtccgggctctggttccctgtgagaagctgccacggcccaccgagatgtccaggcaccacagccgcttcgaaagagattaccgcataggctgggaccgccgtgagtggagcgtcaatgggacccacggggccaccagcgtctgcagcgtcacctccggtgctggtggcagcacagccagcagtctcagcgcaagaccgggcctcctaccactgcctgtggtaccctcccggctgcccacacccgccacggctcctgctccctgcacgaccggcagcagtgaagctatcaccagtcttgtggtcagctcggcctccgcagccaccaccaaggctcccggcatctccaaagctgacaatcagtctcaaggactcacgaccagcatccgatgggggcagacgcctgtcaatcagtccacgccctgggacactgatgagccgccctccaaacagatgagggagagcgacaatccaggcacagggccatgggtgaccacggtggccgctggaaaccagccttccctgatcacacactcctatggagtgacccagcctcccaccttcagtcctgctgtgaacgtccaggcccccgtcatcggggtgactccctcactgcctccccatgtgggaccccagctcccgctgataccaggccactactcactcccacagccgccctcccaaccgctgagcagtgtggtggtgaacatgcctgctcaggccctgtatgccagccctcagcccctggcagtatccactttgcctggagtagggcaggtatcccgcccgggacccacccctgtgggcaacggccacatggcagggcccctgctgcctccacctccaccagcccagccgtccgcagctctccccagtagtgttcctgccaccaatgggccgcccacaacagactcggcccatgggctgcaaatgcttcggaccattggagtggggaagtatgagttcaccgacccagggcaccctaaagaaatgttgaaggaattgaaccagcaacgcagagcgaaagcatttacagacctgaaaattgttgttgaaggcagagagtttgaagtccaccaaaatgttctagcttcctgcagcttgtatttcaaggacctgattcaaaggtcggtgcaggacagcagccagagcagccgggagaagttggagctggtgctctccaacctgcaggctgatgtcctggagctactgctagagtttgtctacacggggtcgctggtcatcgactcagccaatgccaagactctactcgaggcggccagcaagttccagtttcacaccttctgcaaagtctgtgtgtcctttctagagaagcagctgactgccagcaactgtctgggagtgctggccatggctgaggccatgcagtgcagtgagctctaccacatggccaaggccttcgcgctgcagatcttccccgaggtggccgcccaggaggagatcctcagcatctctaaagatgacttcattgcctacgtctccaatgacagcctcaataccaaggccgaggagctggtctacgagactgtcatcaagtggatcaagaaggacccagcaacacgtgcacagtatgcagcagagctcctggctgccgtccgcctcccgttcatccatcccagctatctgctcaatgtggtggacaacgaagagctgatcaagtcatcagaagcatgtcgggacctggtcaatgaggctaaacgctaccacatgctaccccacgcccggcaggagatgcagacaccccgaacccggccccgcctctctgcaggtgtggccgaggtcatcgttttggttgggggccgtcagatggttgggatgacccagcgctcactggtggctgtcacctgctggaacccacagaacaacaagtggtaccccttggcctcgctgcctttctatgaccgcgagttcttcagtgtagtgagtgctggggacaacatctacctctcaggtggtatggaatcaggggtgacgctggctgatgtctggtgctacatgtccctgttggataactggaacctggtctccagaatgactgtccctcgttgtcgccacaatagtctcgtctatgatgggaagatctacactcttgggggacttggagtggccggcaacgtggaccatgtggagaggtacgacactatcactaaccaatgggaggcagtagcccctctgcccaaggcggtacattcggcagcggccactgtgtgtggtggcaaaatctacgtgtttggtggcgtgaatgaggcaggccgagcggcaggtgtcctccagtcctatgtgccacagaccaacacgtggagcttcattgagtccccaatgatagacaacaaatatgcacctgctgtcaccctcaatggctttgtgttcatcctgggaggagcttatgccagagccaccaccatctacgacccagaaaaaggcaacatcaaggcggggccaaacatgaatcactctcggcagttctgcagtgccgtggtgctcgatggtaaaatttatgctactggaggcatcgtgagcagtgagggaccagccctgggcaacatggaagcctacgagcccacaaccaacacatggaccctcttaccccacatgccctgtcctgtgttcagacacggctgtgttgtgataaaaaagtatattcaaagcggctgacgtcagcagaaagcccaagacaagtctatgggacaagtccagtgaggcgaatgccacacaacatggttatctttcaacgccaacgagaggcccacaaaacacaatcgagaaactcactgcgctaacatttcgaatattctctacattgaatgtagaaaatcatcctcgccttttgggtgaagaggggcacagtgctccgggcagcaggaacctggaatgcacggagcccgggctcccaggccagcgaggcacttgctctgaacagagggcgctcgctctgcccggtgcaatagtttcacatatttttcaactgggagagaagctgtttttttccttcttgcagagcaagcttgatccctaaaccaccatagatcagttatcctgtgacaatttggggcatcacgctcttttggaataaggtcaaagtgtccttatcactttgattcctactttttttttttttttttggagttttttgggttttgtttttgtttttcttaaacctatctatactttcaagtggccgagagaaaaaggaggaatggaactgtgcagcaccaaggcctcggagagcgcctggtgccactagacaggatggagggccagctgcacgtgcagagccagggctggcagccgcaggggtcgctgggaagagcagccagggtcggcagccgccagggtcgctgggaagagccgcctgtccttcatggctttttctaccatgtttttgctgcaaaagaacatggtgggtatgctagctttctcttagccagtatgaattatttagatttccaaggcattttcttgatacacaaaaggctatttttaagtaccgagagaaggaggccacaggagggacaacgctgtgggacttcccaaagctctttgtaggtagcgccagaggtagggcccttgctctcatttttctatgtgcagaatagaggatctctcctgaggtgggccatgccccccactttatttttggaaaaagttaactcccagacagtcctggaagaccatgccatgtggaggcattgtcagagggaagggtctattctagaagaacatggttctggcttctgacaacagtccagctccctccatgaggtaaagctgacgacccaggccccgggaaagggactgagggagggagggagggagaagaaatgtctacaaagcacaggagactatttttgatatttatagctatatattcaaggcacctgccatgagagctctcaggatggggacagccttcttagacgagccatgacagtcacggcctgagaggcacaagcagaaacactcttcttgttgcctttctgcatagtaacctgagaaaagggaaggcaggagaggtcccagcgaggctcaaaagagaaaggccttccctttagtccttagaccctcaaacctcacccggtcatcagtttccattccagggcaggaagaaggccgccgccaccgtgctgtttagttgaagttagtaccaaatacacaccattttgctacctggaaagtcagcttgccatcacatcatgacaagaaccattgacagagagaaagagacactacatgctttcatcattcagtattggatgacccaaaattccagtcctaacgacaggcatcagcctctagcattacaagtgtggctcccacttgacaagataccgagcttcgttatgcagtttttaatattatttattattttaaaaaagtaataagcacaaaactacatacattgtatgtcatttaaagtatttatgtcaaacagggtgcaagtgtgaacccaaggactggagcacaaatttctaactgcctggggcagggcgaatgtgagcattggtgtgtgtctgcctccaagggaggtgttagtggtcaacaagactccgcggtgacactgaaattccgtttctctcctcatctatcacactggagcaaaactggctatttctgtgaatgatataaaacagggttctctgtaatggtattgtatatagtatatgtttattgttaagttgttatattataataaatatatttatagatctagactcggaagccaatgcaatgtctttgtgtgccattgtctttgcctcagtgacactacagtcctgttcttcctcttggtcatcaagagtgtgtatccttctacagatgtaggtttggatgattagagaggacccttttgcaaagggacagagacagatgaacggtaacctccagtaaggaacagctgaaagtcaaaagccgacttagagacttagagataccatggagttgatgcagcctgttccctgagctcctatgttcttcttctcacagcatgtaaaagccccacagcttgcttttgcccccttcacttctgcccaaaggcccccttttcttattttctcaattcatcagcctggagctacaagacacttcctccaggaagaaaatcagtgatttccatgctgagacccaggcagggtagccttcctgcttcctcagttgtgtctaaagggttctctggtctatcacacagacccgctttgccatgcccaggacacctaggcaagactgcttagcatgaaatacggaaatgcatctgtcaggaaggaagtttaggaagaaagggtagattttccagcatagtcagccacattaagcctccatgtcctccatccctaaatcgtaccaagacctagtgtctccttgaaagcaaaaccttaataagcaaaaaccagcacaggaggtgagaggagaggttctactggaaactctgttgaaacccttgggacaagaggaccattctcctgtcaaatcagtactttcctaggaacaactgtcctacaagggtggacacagactgtctagaagaagccaatcgtgaaggggaaggtgataatctgtgataaaaacagtctgccagctccattattaactatttcatttgtttaagtgaaggaacatgttcttgtggtccatgaagaatctctgtgttttatacctaactctgtgtggtaaagagatcccattgtaccatagacagacagctgagcacacgggagcaggagacccgccatggggactgtcaagacacggctgtctgatctaacaaagatgatgagaacagtgcacactgtgtgcatagattttttattttaaagaacttcaaaataagagtgcaatcaggggaagtacctcctgtgtgataaacagtatcttcattaaagaaaataggtaatgtaattggaaaatattagttaagaaataggtgttaggcctaaaagaaaaaaaaaaggtattagcaaacggaatccaaaagtaaaaaaccaagttcaacactaaaatctggcatgaatcggtaacacctcatagcttttgaataaccataggggaatctgtgctcaggaggagcaggagactccaacgtagtcctgagggctctgcagagggacagacagcaaggccgcaggcaccgtgtatcatgagctggaggagcactgagaaatcacttctagacagccataaagaaatgacatgaactctgggaaaataatgccaactattctattacaatggcgcgtgaaaagtgaccggtacttgtttgtcgacactaatgatgcgctgaccaccatgctaagctctcaaagccctgaatcctcaagattcaggtacaaaagtcatacacacaaaaa

SEQ ID NO: 53 - DNA, 1716 bp, Homo sapiens
cgggaggcggctagtgagcgggcgggcgtgggctgctggtggccggacgggtcacgctgcgggtttcgtcgccgccgccgccgccgccttgcaccattgcaccatgtccggccagctggagcgttgcgagcgcgagtggcacgagctggagggagaatttcaggagctgcaggagacacacaggatctataagcagaagctggaggagctgacctccctgcagacgctgtgcagcacctccatcagcaagcagaagaggcacctgaaggacctgaagcacacactccagaggtacaagcgccattccagccatgaggaggcggcgctcatccaacagatgaccgccaacattaaggagcgacagaatgtcttctttgacatggaggcctacctaccgaagaagaatgggctctacttgaatctggttcttggcaacgtgagcgtgaccctcctcagcaatcaggccaagtttgcttacaaggatgagtatgagaagtttaagctctacctgaccataattcttcttctgggtgctgtggcgtgtcgatttgtccttcattacagggtgaccgatgaagtcttcaacttcctgctcgtgtggtattactgcaccttgaccattcgagagagcatcctcatcagcaatggttccagaatcaagggctggtgggtgtctcaccattatgtctccacgttcctgtctggagtgatgctgacttggcctaatggactaatttaccagaagtttcgaaatcagtttttagcgttctccatttttcagagctgcgttcagtttctacagtactattaccagaggggctgtctctacagactgcgggccctgggggagaggaaccacctggacctcacagtggaagggttccaatcctggatgtggcggggcctcaccttcctcctgcctttcctcttctgtggccatttctggcagctctacaatgccgtcaccctgtttgagctctccacccatgaggaatgcaaagaatggcaggtattcgtgctggctctcaccttcctcatcctcttcctgggcaattttctgaccacgctcaaagttgtccatgccaaactccacaagaacagaaacaagacaaagcagccatgaacctccggacccttgtgccacctgggggccttgggacctaagactgctgggcgttccagtcagcaccctggctgccggtcagtggtagccatgggcagcaacatctcaggggccagcagcatcaccgggagcagggccaaggccccggtccccggcggacatgaggaatgtgaaggccacctgcgcgcgcagtattaacctgtcccagctccctatttataacatatttaatatcgggtcttccctgcttccctggaatccgaggcctgccttccctggagctctgtgtaatggccgtgggcaggcctgtgtctgtctttcagggaggggccagaagcctcagggaacccctctcttcctactcctatcatttgaatccctcactggggtttagatgtgcctcactcgagttggctttagccgacctgctactttactgtgtcccaggcaccgggaggaatgaaactttgagcatcctgtggagcctcccctgtcctgggccaccacctcttctgtaagcctcccacagccccaccttctgcggtggacaaatatttaagttctcagagaacctaaaaaaaaaaaaaaaaaaa

SEQ ID NO: 54 - DNA, 2943 bp, Homo sapiens
cccacgcgtccgcccacgcgtccggtgcatcttaaccgaccattgtcttgaattctagagtggatcatgacccatgaagagcatcatgcagccaaaaccctggggatcggcaaggccatcgccgtgttgacctctggtggagatgcccaaggtatgaatgctgccgtcagggctgtggtccgagttggtatcttcacgggcgcccgcgtcttctttgtccatgagggttaccaaggcctggtggacggtggcgagcacatcagggaggccacgtgggagagcgtgtctatgatgcttcagctgggtggcacagtgattggaagtgcccgatgcaaggacttccgggagcgagaaggacgactccgggccgcccacaacctggtgaagcgggggatcaccaatctgtgtgtcatcggaggcgatggcagcctcactggggctgacactttccgttcagagtggagcgacttgctgaatgatttccagaaagacgggaagatcacagccgaggaggctacaaagtccagctacctgaacatcgtgggcctggttggctcaatcgacaatgacttctgtggcactgatatgaccattggtactgactctgccctgcacaggattgtggagatcgtagacgccatcaccaccacggctcagagccaccagaggacgtttgtgttagaagtgatgggccgccactgtggatacctggcccttgtcacctctctgtcctgtggggccgactgggtttttattcctgagtgtccgccagacgatgactgggaagaacacctttgtcgccggctcagtgagacaaggacccgtggctctcgtctcaacatcatcattgttgctgaaggtgccatcgacaagaacgggaagccaatcacctcagaagacatcaagaacctggtggtgaagcgccttggatatgacacccgggtcactgttctgggacatgtacagcggggtgggacaccatcagcctttgaccggatcctgggcagcaggatgggtgtggaagcagtgatggcacttttggaggggaccccagacaccccagcctgtgtggtgagcctctctggtaaccaggctgtgcgtttgcccctcatggagtgcgtgcaggtgaccaaagacgtgaccaaggctatggatgagaagagatttgatgaagccattaagctgagaggccggagcttcatgaacaactgggaggtatacaagcttctagctcatgtcagacccccagtctctaagggtgggttgcacacagtggccgtgatgaatgtgggggccccagctgctggcatgaatgccgctgttcgctctaccgtgaggattggccttatccagggcaaccgggtgctggtcgtacatgatggctttgagggtctggccaaaggtcagattgaggaggctggctggagctatgttggaggctggactggtcaaggtggttccaaacttggtactaaaaggactctccccaagaagaacctggaacagatcagtgccaacataaccaagtttaacatccagggccttgtcatcattgggggctttgaggcttacacagggggcttggagctgatggaaggcaggaagcagtttgatgagctctgcatcccgtttgtggtcattccggccacggtttccaataacgtccctgggtcagacttcagcatcggggctgacacagcactgaacaccatctgcacgacttgtgaccgaatcaagcagtctgcagcaggcaccaaacgtcgggtgtttatcatcgagactatgggtggctactgtggctacctggccaccatggcagggctggccgctggggctgatgctgcctacatttttgaggaacccttcaccattcgcgatctccaggtgaatgttgaacatctggtgcagaagatgaaaacaactgtgaagagaggcctggtgctgaggaatgagaaatgcaatgagaactacactactgacttcattttcaacctgtactctgaggaggggaagggcatctttgacagcaggaagaacgtgcttggccacatgcagcagggtggaagcccaactccctttgacaggaattttgccactaagatgggtgctaaggctatgaactggatgtctgggaaaatcaaggagagttaccgtaatggacggatctttgccaacacccctgactcaggctgcgttctggggatgcgtaagagggccctggtctttcagccagtaactgagctgaaggaccagacagactttgaacaccgaatccccaaagaacagtggtggctgaagctgaggccaatcctcaaaatcctagccaagtacgagattgatctggacacctccgaccacgcccacctggagcacatttcaaggaaacggtctggagaagccgccgtctagacctctctggagtgagggggaaagatcatgtgaccatggtcagctcacaccctggtagatcatggcttctcagtgttctagcccagcccctccccttctaggtttccctgtactccatgcctgcaaccaggatcagctgtggccaggagctggaggaggggcggtgagtgactctcctaggtggctgtccttcctcccttccactccagcttcatcggtcacacaggctgggtctctctagtgctacggctagatttcagctactcaagagttttcctaaaaataagctttatttatttctttgtgataacaaagagtcttaggtccctttactcgcaccacagtgatgaagtgtaactacactaataaatgccagctggccactgtgttttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 55 - DNA, 3600 bp, Homo sapiens
ggtttaccggcggagcggccgttataaattataggaagattctgccaaagactgaactgtcttgactttcagatactgcatagttcaaggtcagcctcaattcctgggtctcatgaatgaaacaatggctacagattctcctcggagacccagtcgttgtactggcggagtcgtggtccgccctcaggccgtcacggagcagtcctacatggagagcgtcgtgacttttctgcaggatgttgtgccacaggcttacagtgggtctcccctaacagaagaaaaggagaagatagtctgggtcagatttgagaatgcagatctgaacgacacatcacagaatctagaatttcatgaactgcatagcactggaaatgagcctcctctgctggttatgatcggctacagtgacggaatgcaggtctggggcatccctatcagcggggaagcccaggagctcttctctgtacgacatggtccagtccgagcagctagaatcttgcctgctccacagttgggtgcccaaaagtgtgataactttgctgagaaaagacctcttcttggtgtttgtaagagcattggatcttcaggcacgacaccaccgtactgctgtgtggatctgtattcactccggactggggaaatggtcaagtccattcagtttaaaacacccatttatgatcttcactgtaacaagcggatcctcgtcgtagtcttgcaggagaaaattgctgcctttgatagctgtactttcacaaaaaaattttttgtcacaagttgttatccttgtcctgggccaaacatgaatcccattgctcttgggagccgctggcttgcttatgcagaaaataagctgattcgctgtcaccagtcccgtggaggagcctgtggagacaacattcagtcttacactgccactgtccttagtgctgctaaaacactgaaaagtggcctgacgatggttgggaaagtggtgactcaactgacaggcacactgccctcaggggtgacagaagatgacgttgccctacactgcaactcaagacggagccccttggttccaggcatcatcacagtcatagacactgagacggttggagagggccaggtgcttgtaagtgaggactcggacagtgatggcattgtggcccacttccctgcccatgagaagccagtatgctgtatggcttttaatactagtggaatgcttttagtcacaacagatactcttggccatgactttcatgtcttccaaatcctgactcatccttggtcatcatcacagtgtgctgtccatcatctgtacacacttcacaggggagagaccgaggccaaagttcaggacatctgtttcagccatgattgtcgttgggtggtggtcagtaccctccggggtacgtcccacgttttccccatcaatccttatggtggccagccttgtgtccgcacacacatgtcaccacgagtagtcaatcgcatgagccgtttccagaaaagtgctgggctggaagagattgaacaagagctgacatctaagcaaggcggccgctgtagtcctgttccaggcctgtccagcagcccttctggctccccgctgcatgggaagctgaccagccaagactcttacaataactttacaaacaacaaccctggcaatccccggctttctcccctccccagcttgatggtagtgacgcctcttgcgcaaatcaaacagccaatgacactgggaaccatcacgaagcgaacagggccttatctctttggagcggggtgtttttccataaaggctccatgcaaagttaaatcacctccacaaatttctccaagcaaatcaatgggcggtgagttttgcgtggctgcagtgtttgggacttccaggtcgtggtttgcaaacaacgctggtctgaaacgggaaaaagatcagtccaaacaagttgtagttgaatctctttacatcatcagctgctacgggaccttagtggagcacatgattgagccccggccgatcagtactgctcccaagataagtgacgacacaccactggagataatgacgtcaccccgtgccagctggacacttgttagaacgcctcagtggaatgaactgcagccgccattcaatgcgaatcaccctctgctcctcgctgcagaagcagtgcagtattaccagctcctgctggccggctctcttccccctggaagtcctggccccattactcgacatgggtcctatgacagcttagcctctgaccatagtggacaggaagatgaagaatggctttcccaggttgaaattgtgacacacaccggaccccacaggcgcctgtggatgggcccacaattccatttcaaaaccatccaaacctcaggccagaccacggtcatatcaactagttcttctgtgttacaatcgcacggacctagcgacacaccacagccgctcttggactttgatacggatgatctagatcttaacagcctcaggatccagcctgtccgctctgacccagtcagcatgccagggtcatcccgtgctgtctctgatcggaggggagtttccacagtgaccgatgctgcctcaggtacctttgaccggagcgtgaccctgctggaggtgtgtgggagctggcctgagggcttcgggctgcggcatatgtcctccatggagcactcagaggagggcctccgggaacggcttgcagatgccatggccgagtcaccaagccgagacgttgtgggatctggaacagaacttcagcgagagggaagcatcgagactctgagtaacagctccggctccaccagcggcagcatcccaagaaacttcgatggctaccgatctccgctccctaccaacgagagccagccgctcagcctcttccccactggcttcccctaggcaccagccacctgcttctgactggctggcccgctcacccctagagggactgactgggaggggggagtacccccatctccctacaccaccaccaccacccctgtcccgcacgcctctgttcccacccttttcagtctcctcaaccccaccttccctaagcttagtggcaacagctgttcatcccaaactggacgagaagagaccttcagaacacctcggtacaccctgacctcccccactcctgtcagtgggggctgtggcccaaagagactgcagaaactcagtcccttctctctttgcacacaacgtcccgcactggatccgcttttgtattttctaactactcacaggggactggaacaggggccagatcttagggcctctggtaggggagagttgaaagatgcagccaggcattttggtggacatcagccctagcccagttcaggctgggatgcacattgcccccaggtacccctgctcagttctaaaacaggagtgaactgagacctagagcgatagagaggaagaaggaaagaggcctccccttgcccttgcttccctgttggctcatggagagatttgtctgtcttatctttaagcccttgtcccctggtcctgtactgttccatgaagggaagtgcagttaccgaggccgtgtcaaaagtgcacgtcttgtccaataaatctcgctgcaattg

SEQ ID NO: 56 - DNA, 1517 bp, Homo sapiens
cgcagacggagcgaggcggcggcggcggcggggcggcgcagggcgcggggcggcatggccaccaccgcgcagtatctgccgcggggccccggcggcggagctgggggcacagggccgctcatgcatcccgatgccgccgcggcggcggcagcggcggccgagcggctgcacgcgggggccgcgtaccgcgaagtgcagaagctgatgcaccacgagtggctgggcgcgggcgcgggccaccccgtgggcctagcgcaccctcaatggctacccacgggaggaggcggcggcggcgactgggcgggcggcccgcacctggaacacggcaaggcaggcggtggcggtaccggccgagctgacgacggcggcggtggcggcggtttccacgcccgcctggtgcaccaaggggcggcccacgcgggcgcggcatgggcacaaggcggcacagcgcaccacttgggccccgccatgtcgccgtcgcccggggccggcgggggtcaccagccccagccgctcgggctgtacgctcaggcggcctaccccggtggcggcggcggcggcctggccgggatgctggcggcgggaggcggcggcgcgggacccggcctgcaccacgcactgcacgaggacggccacgaggcacagctggagccgtcgccaccaccgcacctgggcgcacacggacacgcacacggacatgcacacgcgggcggcctgcacgcggcggcggcgcacctgcacccgggcgcgggcggtggtggctcgtcggtgggcgagcactcggacgaggatgctcccagctccgacgacctggagcagttcgccaagcagttcaagcaacgacgcatcaagctgggcttcacccaggccgacgtgggactggcgctgggcaccctctacggtaacgtgttctcgcagaccaccatctgccgtttcgaggccctgcagctgagcttcaagaacatgtgcaagctcaagccgctgctcaacaagtggctggaggagaccgactcgtccagcggcagccccaccaacctggacaagatcgcggcgcagggccgcaagcgcaagaagcgcacgtccatcgaggtgggtgtcaaaggcgcgctcgagagccactttctcaagtgtcccaagccgtctgcgcacgagatcaccggcctggccgacagcctgcaactggagaaggaggtggtgcgtgtctggttctgcaaccggcggcagaaggagaagcgcatgacccccgcggccggcgcgggccacccgcccatggacgacgtttatgcgcctggggagctggggcctggcgggggcagcgcgtcgccaccttctgcgcccccgccacccccgccggccgcgctgcaccaccaccaccaccacacactgcccggctctgtgcagtgaccctgcggactgggttccccgccggcgcagcggtgcctccggcgcgcagttagcgcgcgcggcctggactctttttgttgtttattcggttttgctttggattttacaaaaag

SEQ ID NO: 57 - DNA, 2465 bp, Homo sapiens
ttttgccaagctccgggacttcatcacgttgcgcctgcctcctggcttcccagtcaagattgaaatcccgatttttcacatcctcaacgcccgcatcaccttcgggaacctcaatggctgcgatgagccagtgccctcggtgcgaggtagccccggcagcgagacgccctcccctggcagtgactcttccagcgtcagcagctccagctctaccacctcctgtcgtgcctgtgagatctcccctgcactgttcgaggccccacgtggctacagtgtgctgggcggccagcgagaggctgtgcctcgtgatgaagatgatgacctgctgcggtttgccatccagcagagcctgcttgaggcgggcagtgagtatgaccaggtcaccatctgggaggcgctaaccaacagcaagccaggcacccaccccatgtcctatgaaggccgccgacaggacaggtcagtgcccacgaagccaaagagagcctagaggtgcctgttccctctcttaccccagacccacatgcacagagatcctcactaagccatcatactgcacagtgacactggaaccctactaggcccagagaaccctcagtgccttcaacacctggtatcgagaacccgttatcaagatggcttctgccaaccccctttcctggcacagggagggtggccagtgctccaggcatgtgcagagatcagggacctagctccctggaccttcagaccctcaacaacagacaggttggcgggacctgggatctgccatcactgggtggggcccagacagcccagcgagactcatggcttccccaagagggtgggcaagcctgcccagccctgggacctccacgatgtctcctggccccgcttacgagcaggtgtcccaggtgtgccgggagcggggagcgctcgacaaatcctccccgcccccaggagcgccccacccacgccgcagcgccagcccatgcccccggctccagtgcccagccctcggcccagcccaggtcccggctccagcagccatgtgttccggagctacgacgagcagctgcggctggctatggagttgtccgctcaggagcaggaggagaggcggcggcgagtgcgccaggaggaggaagagttggagcggatcctgcggctctcgctgaccgaacagtagcgcctcctgctgggaccctcgcccactccacgcaggacccgggtctgcgcacctgccgagcggttgctggagtctggagccatcgcattgcgggtgtagcagcgcatcaggcttgagatgccaggggtttgatacaggcgtggaatcactttccaggccagagccctagaggggtgctggtctccctgctttgctgtatggtgacccctaatccgcttctctgggctcagatctgtccaagggcggaggcaggtggtattgaaaaggaaacgaatccttgagaggagcactgtcccgtcccttgctccttatggctggtcctccttcttgctcactgacccctctccaggccagagccctagaggggtgctggtctccctgctttgctgtatggtgacccctaatccgcttctctgggctcagatctgtccaagggcggaggcaggtggtattgaaaaggaaacgaatccttgagaggagcactgtcccgtcccttgctccttatggctggtcctccttcttgctcactgacccctctccaggacactgtccctgaagccttcgcatctcagctcttcacccctgggaggcagctgatcgccctgcgtgctgtgtcgcgctgctttgtcccagacacaaaccagcacgtcaagggcctagctcctccccaccccggcatttcagcgtcaagtgcacttagcggggtacccagcccccccagtcccaacacctgtcctgtgtctcagggtggtcctagcttctccttaggcagccaggaattggaacccccatgcccgcagcacgtttttcttatgatcagggagtgtgcctaaggtgccccctagtttgggcaaggcctggttccctcacggtgccttggggaatgggggcagcatattggcttgggggcaagcactagaaccgacacaaaagggttgatgagggccctggcaccactgtcaccacccaccccttcctaccagctgctgctagccctctgtggttgtgcacccctcttgcccccattcaggggccgactaacaccctttatccaatggtgctaaccccggctctcccttttccaccctacccacctaaagaagcacaggccccacaaagcaggggccacacccttgtcctgggctgccttctggctggcttctctctgccaggggctcagaaggggcacaaaagggggtcaagaaaagagcaacaaagcaaaattgttcttcctcttcccttccctgatgccaggggcaccagactgattctgaggcacaaataaaagcggtttcaaacc

SEQ ID NO: 58 - DNA, 1259 bp, Homo sapiens
gccccaagcccttccgttccgctactgaatgcttgctgaacagatgtttggacgcactttgcccatccttgcaggttaactgtgagtggacatcatcgaagggggcagcctgcgcacccccctccaggagctacatcagatgatcctgacacccatcaaggcctacagctctcccagtaccacccctgaggtgcgctgccgggagccctcactgatggagagcccaagccctgactgcaaagacagcgctgcagcagtcaccagcgccacagccagtgcctcagccggtgccagcggtggactgcaaccaccccagctgagcagctgcgacggagagctggctgtcgcccctctgccggagggggacctcccggggcagttcacccgagtcatggggaaagtgtgcacacagctcttggtctccagacccgatgaagaaaatataagctcctatctacagctcttagacaagtgtcttgttcatgaggcatttacagagacacagaaaaaaagattgttgtcatggaaacagcaggtgcagaagctctttcggtctttccctcggaaaacccttctagacatatcaggatatcgacagcaaagaaatcgtggctttgggcagtccaactccctcccaacagccagctctgtgggcagcggcatgggcagacggaacccacgacagtaccagatcgcatctcggaatgtcccttccgcccgccttggtctcctgggcaccagtggatttgtcagctccaaccagcgccacaccgctgccaaccccaccatcatgaaacaaggaagacagaacctctggtttgccaaccctgggggcagcaacagcatgcccagccgcacccacagctccgtccagaagacccgctcgctgcccgtgcatacttccccacaaaacatgctgatgttccagcaaccagaatttcagcttcctgtgactgaacctgacatcaacaataggctggagtccttgtgcctcagtatgacggagcatgccctgggagacggggttgaccggacctccacaatctagaaactgaagatgacagtgaccgcgctggccgtgaaatggactgctgcgggcccagtgtcagccatcttcagggttgcagagactcttccaagacactttgcagcctttttcccctggtccctcgcccattttgattttgtgagagcgtaggtcatccttgtaaacatatcagtagacctgcaaaaaaaaaaaaaaaaaaaaaaaaaaaaaa

SEQ ID NO: 59 - DNA, 10040 bp, Homo sapiens
tccgtcgttgctgaggatcgctcttagttcttgagccgccttgtgccgacttgctcggtttgtttggggagataacttgttttgcctcagcccgcatcccctttccttgaccccttgcaatcggatgttctagatgactgaatggagttttgagttggacttttgtgtccctgccgaaattgggcctgatcccagagcactgggggtggggtgtggaggtgttactgtaaaatgcaagttggataaaaagaagacctctcgccaagggccccaatgagtggcacacagtctactatcacggacaggtttcccctcaaaaaacctataaggcatggaagtattttgaaccgagagtcaccaacagataagaaacagaaagttgaacgcagttcatcgcatgattttgaccccacagatagctcctccaagaagacaaagtctagttcagaggagagtagatccgagatctatggtcttgttcagcgctgtgtgatcatccagaaagacgacaatggcttcgggctgacagtcagtggagacaatccggtcttcgtacaatctgtcaaagaagatggagcagccatgcgtgctggagtacaaacaggtgatcgaatcatcaaggtgaatggtaccctggtgactcattcaaatcatctggaggtggtgaaactaatcagatctggttcctatgtagctctcactgtccagggacgcccacctgggtcgccccagatcccacttgctgactctgaagtagagccatcagtcactggacatatgtctcccattatgacatcccctcattcccctggagcagccgggaatatggagagaatcaccagtcctgtgctcgtgggggaggaaaacaatgtggtccataaccagaaagtagagattctgagaaagatgttacagaaggaacaggaacggctgcagttgctgcaggaagattacaaccgaacggctacccaaagattgctcaaggagatccaggaggccaagaagcacatcccgcagctgcaggagcagctgtccaaagccacgggctctgctcaggacggagctgtaattgcaccctccaggccattaggtgatgccctaacactcagtgaggcagaagcagatcctggagatggcctgtgcaggactgactggagcagtggagatgcttctcggcccagtagtgacagtgctgatagtcccaagagtagcctgagagaaaggagttactcggaggaagccccagagagaagtgaaggagttcaggacgctgagccgcagtcgctggtcggcagtccctccacccggggcgcccctcacatcattggagcagaggatgatgattttggtactgagcatgagcagatcaatggacagtgcagctgtttccagagcattgaattgctgaagtctcgccctgctcacttggctgttttcttacaccatgtagtctcacagtttgaccctgcaacattgctctgttatctctattcagacctgtataagcagacaaactccaaggagactcggcgtgtcttcctggagtttcatcagttcttcttggaccggtctgcacacctgaaggttccagttcctgaggaaatctccgtggatctagaaaagagaagacctgagcttattcctgaggatctgcatcgcctctacattcagacaatgcaggagagagtccaccctgaagttcagaggcacttggaggatttccgacagaaacgcagcatggggctgaccttagctgagagtgagctgactaaacttgatgcagagagagacaaggaccggggaactctggagaaggagcgagcatgtgcggaacagatcgttaccaaaattgaagaagtgttgatgactgctcaggctgtagaagaagaaaggagttccacaatgcagtatgtaattctcatgtatatgaagtatttgggagtaaaagtgaaagaacctcgaaacttggagcacaagcggggtcggatcggcttccttccaaagatcaagcaaagcatgaagaaagatagagagggcgaagaaaaagggaagcgaagaggattccccagcattttgggacccccaagaagaccaagccgccacgacaacagtgcaattggcagggccatggagatacagaagtcacgccacccgaagcacttatccactccctcgtctgtgagtcctgagcctcaggaccctgccaagttgcgccagagtggagtagccaatgaaggaacagacactggatacctgcccgccagttccatgtcctctgccacctcagggactgctctttcccaagagggagggagagagaatgatacaggaacaaaacaagtgggagaagcatcggcgcctggagactgcttggacagcacacctcgagtccccactactgttttcgacttccctcctcctctattagatcaagtgcaggaggaggagtgtgaagtagagagggtggcagaacatgggaccccaaaaccctttcggaagtttgacagcatcgcctttggagaaagtcagagtgaggatgagcagtttgaaaatgacctggagacagatccacccaactggcagcagcttgttagcagagaagtgttactgggactgaaaccttctgagatcaaaagacaggaagtgattaatgaattgttctacactgagagagctcatgtccggacactgaaggttcttgaccaagtattctatcagcgagtgtccagagaagggattctatcaccttcagaactacgcaagattttttcaaacttggaagatatccttcaacttcatgttgggttgaatgagcaaatgaaagctgtccgaaaaaggaatgaaacttctgttattgatcatattggggaagatctgctcatctggtttagtggaccaggagaggagaagctgaaacatgctgctgccacattctgcagtaaccaacctttcgccctggaaatgatcaagtctcgccagaaaaaggattcacgattccatacttttgtgcaagacgctgagagtaaccccctgtgccgtcgtcttcagctgaaggacatcattcccacccaaatgcagagactcactaagtacccgcttcttttggataacattgccaagtacacagaatggcctccagaaagggaaaaggtgaagaaggctgcagaccattgtcgtcagatcttaaattatgtaaatcaggctgtcagggaggcagaaaacaagcagcgcttagaagactatcagcgtcgccttgacacctccaatctcaagttgtccgagtacccaaatgttgacgagctcaggaatttggatttaacaaaaagaaagatgattcatgaagggccgttggtctggaaggtgaatagagataaaagcatcgatctttatacactgctgctggaagacatccttgtgctgctacagaagcaggatgacaggctggtgctaaggtgccacagcaagattctggcatccacagccgacagcaaacacaccttcagccctgtcattaagctgagcacggtgctggttcggcaggtggcgacagataacaaagctttattcgtcatctccatgtcagataatggagcccaaatttatgaattggtagcacagacggtttctgaaaagacagtctggcaagacctcatctgtcgaatggctgcgtcagtgaaggagcagtctacaaagccgattcccctcccacagccgccaccttgcgaaggagacaatgatgaagaagaaccagccaagttaaaagtggagcaccatgatctttcagtcgctggtctgcaaagtccagacagagttttgggattagaatctcccttaatttcgtcaaagcctcagtctcattcactgaatacccctggcaaatcagcggcagaacatctctttgtgaccgcaacacagtttgcaaaggaacagcatgcaaatggggcactcaaggaaggtgatggcgggtatccagtcacaatcccaggtccacacttgcctgtgtcagaggagcggtgggcattggacgcactgagaaatttgggcttgttgaagcagttgctggtacagcagttaggtctcaccgagaagagtactcaggaagactggcagtccttctcacgatacgggccagcttctgaagaggtacaggcagacagtggcatccgggacttggaaaatgttaaggcttgtcatgccagggaaggacagatgtcttttaaaactggaactggtgatattgcaacttgtgacagtccacggacttcaactgagtcttgtgctgcacaggattcagtgatactggcatcccaagacagccaggcaagtaacgttttagtaatggaccacatgatcctgaccccagagatgcctcctgcagagccagaagggggtctggatgagagtggagagcacttttttgatgcccgtgaagcacatagtgatgataatccatcagaaggtgatggagcagttaaaaaggaagagaaggatgttaatttacgcatctcaggaaactgtttgatccttgatggctatgatgcagttcaggagagctccacagatgaggaggttgcctcctcgttccccctgcagcctgtgacgggcatccccgctgtggactccagccaccagcagcaacattcccctcagaatgtgcatcccgaggggccggtttcaccattcaccccagaattcctggtccagcggcactggcgagctatggaggatacctgttttgagatccagagtccctcatgtacagattcacaaagccaaatcctggagtacattcataaaatagaggctgacctggaacacttaaagaaggtggaggaaagttacgctcttctttgccaaaggctggctggctcagccctccccgacaagctctcagataaaagttagagccccgtgtcctggaggtgactgcagcttgttggattttgagtgtcggccctgtatcaaccacatcctggctccagtgtggacacagagagagtgcgacagagggatctgcctggaaccacctggcctcagccaggccccaggggccccgcggggactagttcttccttctggcagctgcacacatctgtcagtgagggaatgccaggtctctcacgcctctcctcactatcagaaatgcattttgattcagagtaaaaaccaaatgtatagagctttgggtgtaggatataaaattgtacgtaggtaagaaaaagagaaaatcagatgtatttatttgacttcattccgtatttgaaagcacactttaatttttactttgccttgttttgtttttaattgagtagtgagagttttagccctttgtatttagcacacccaaggatcaattgttcctgaagccgaggtcaggctggggatggagaggttagtagacaggcagtgcagaagagcaaagaggactacctggccctcgctaatctgtaacccagccctcctgtccagaaggctgcctttctgaggatcatctcagaggtgctgcatgactcctgtgtgtggatgccatgagctgtgtgtgcccgggtgctcggagcaggaggacctcctcccagcccaaggcgagcatctcctactgggttccattcccaggaaacaggaatcctcagtacactaaatggcaggcacttgaaaatgggtggatttcccttgtcgttgtcttttctattgatagttgggatttgggagagaagaaggaggttttcgaaactgaatcactactgagttgaaccatggctgtcactagccacgggtactgctgattataaggccagtaagaaaattagtacctggatgtttgactcttaaggttttgcactgatggtgccaaagggctctgagtcacacgcagagccaagggagggcagaggaaggcatgaaggagagttgctcgcatcgcatcccttcagagagctttctctgacctcactgccatagccctgtctgtctggtgccaggactggccttgggaacggggaccagcggtgagcagttgcagccgccaaggtgctgacgtgctgcagtgtggacagcgcaggtggaaggccagctggtcagactgtggcctgacaactcttaattctgatggtgttcagtggcttgctttgtgcatttgataaatatgtttttagagtgttctgtaaaaaaaaaaaatcatttgtctaatctaaattctgttaagaaggagctcacatgttgcttacatcttgcaggagggggcctctgaactctgggggcagtcaggatgtaattttattctccccttaatgcccctcacacaaaggaagctcactgtcgccaagaatagtcccccagctgcttttatgaacacctctacaacttaacaagctcatctgtctgggtccagattctcgtttctgtcctctaagctcttacgtcgattggctaggacccctgtacctcatctataaattggaatcatattttgagtcatagtcaaccctaactcataatggagctttgagattttggagtagaatctaatatataagtgaagtttgggatttggacctccctgtatctcttaggagaagcaagtagagaccgaatggttccgttgtcttccaggcagcctcttacactacaccatctgggtagctcatcagacagcagtgttccgacctcagacactacagatccctccgtggttctccagcacagttcagccggaactctgttctcctgcttgtttacttccctctttctgagtcccagcctctcagagtcacagcagggcttactggacaggtactctggcgttgctccagtacttccaaataaatgcatatcattacaccttcagatgaaacctcagaatgaagtgcattctgaaagacaacagcaacaaaccactttgtaagttttttttttactctaccagccttacttcttgcctgttcccctccctcgggcctcctgtgccttccactccattctggccattagaatccatttgactttgacattacatctcccttccgtggtggcgacagcagtgttcctcctgttctggaatgagatggtcattgtcatagtcctcttaaaggtgggtgacaagaaggtgcagtgtacttctgctccaaagaacactaggcaagcggaaagccatgtgtgccatgggtgcaacctgtgaccctgctctcctgtgagcttagtggctctcctgtcactaactggcaggttatgtgtggagtgtcaccttttcagagagaaaaactaaccaagccaaaccagcaaaccctccaagtactgtaatgtaaaggccggctttggcttccccacctgtatagtttcatggcagccccctggactgttgagttctgctgctttcacaacctaaactgtaactatttattgtatcacacgcaggttgttcatgagtattttttcctttgtttccccaaacaacttcttttttttcatgaagtcctggaagcttatggtttattgtggattgttctcaggagagtaagagacaagaaaagcaacatatatgtggggggaggggttgaagtggtggtggagcaaagccagctggagacaggctccagcctagacccttgaagaccagagagcagcaatgcaccatactcttgatttcatggggagaaagggggatggaaccccaggccaggaggtgtaactcctagctgcacctgtttccatatgaccaagagtaatcatccgggaatgagagagtctataaagaacactcattgctgctgcaggtgagacagacatggccaggtgacaccagccatgtctctgatttcagtcaactcttttcattagtttcctgaatagcaaacacttccttgaaactcttctcaaacttggactccagtctaagtcccatttcgccaaacttagggtccttgaacgtgattttgtgattccggaagatgagcctcatgtcttgcacaaagtcttctacttggcagtaagcctggtcactcagctttttcttgattatgttcaaccacataggctcttgcacacccacagtcatctctctaaaataataataatatggcatcttggaaaaaaaggagctctcagaacagcaatagactctcaagagaacatactcgcacttcaactgttcctggggagccatctccctctgcagtatctcagattctggaagactcggttgacttcctaaggactgcatcctacagaagatgcaactccatggtgttatctcagcttccactgttgggatatgacagtcctcatggaaggctctggaacaagtatcacagcagaagagtattcctccatcccggcacacctcacattcatctgagttctgaatactagggtcagctggggaacgatgcaagttctgtgttctctgctcttttcttttgcgatgtatccgtggaggattgggtagaaattttctctggatcaggagtttgaggggccaattatggcagcgcaggctcagtttccagttctttgactttccatagcctcccatcatttcaaattccgagggtgtgaaccagtttccattctgacactgtatggacttcacagatatcccttgtttgaatttatccttgtgtaacactcccttcaagttaccacacgtcacgggaagcaactcagcactaaaatcagcattttcttttctgcgtcttcttagacccctgatcttagcagtgcgtcctgggttctttttcattttctttctcttgaaagctgcctggacaacagctattctgccacgttttctcagtacactcattggctttctccatttggtttgaccagttgtggctttcttttttcttctctgcttcttgggttttctgcagaatgttcctcctggtgcccacacattctccttggctgttccatcctgcttccagtcttctggcacatatggaacacatgacacaggaacacttcttttcacgtgtgggttgctgtgctcctgggacaaaattgtgggtgtacataggtttgttatctaattttgtaatactggggaccaagctcagggtctcatttgtgcaaggcaagcagtctaccagtgccccaaacaacttctaatgacctctagaaagaacaagcccttttttagaaagtattctgtttcattgtcttttggggagtgaagaagagaaaagtccttgttgccagaattttagaagctaaaagactttatgctgcctctactgcaggggctcgttttcccgtcgctcctgggcactggcgctggcctgctgcctgcagctggtaccctgcccaagctgtcttctctgcagccgcctgcttgagcttgctctgtgcctgcttccctccacgctccgaaaaccatgatctacaaaaacagacttctgcaaggtctcgctcgcctgcccacccgccccctaacgttgtgtggttttggttttggttttggtttttgacagttcacccccatttagtgtgagctgtaaaactgtactttatttaaaggcgtgctcttgttcccgtctggctgccttccccatgggtgctgagggtttgggtaatacactgatcacggaaaaggtggtcttctcagaaaccagtccctccctcccagccattgccaggtgccacccgctaagcagacatactacagcaggcgaccgtagcgtgcacactgttggtggtttttaatgagcctctctttccccactaggctgtctcactttacttgttcaccatacaatcatttactctttaacaaaaattgcttttaagagaaatctggaactatctttaaaaaaaaaaccttattaataatcatgtatttttactgatcacattttgaaatgcctaaaagacttttattgctctaattatccagatgcacctttgtaaaaagctcttttatgaattagctgataaggctgtatgtttctggaacaaaatattgggtcatctaaaactttctgttttctggggtctgggaaaatagaaaataagaattcaaatattaaataagcttaaaagaactg

SEQ ID NO: 60 - DNA, 2003 bp, Homo sapiens
gggtgacgtgttagccatggagacccaggagggtgaggaagaccgagggtgatggggaaaccgaggctgggacggcgcggttgttcccgtggtccgcatgcgtgggggaggacgcggctcggcctggtgtctccaaacaaagcatacgtgaacggatttgggactacatggaatcccatgatatagctgactttccccgacccgttcatcacaggatccccaacttcaagggtgctgctcaggctgcggggcacctcccacacctgcaagctttccatgtggccagaaccattaaagtcaacccggacgctcctcagagaaacgcccgcttcctggtgctggagagcaaaaaaacactgctggtcccaacaccacgactgcgaacagggctgtttaacaagatcacgccaccccctggagccaccaaagacattctgaggaaatgtgccacctcccagggtgtgcggaatttcagcgtcccagtgggcttggattccagtgttcttgtggatctagttgtggtgggatctgttgctgtgtctgagaaaggctggcgaattgggaaaggggaaggctatgctgatcttgaatacgccatgatggtgtcaatgggagccgtccacaaggggacaccggttgtcacaattgtccacgactgccaggtcgtagacatccccgaggctcttgtggaggaccacgacctcacagttgactacatcctcactcccaccagggtcatcactacaggctgtgcacgcccaaagccaacgggcatcatgtggtccaaggtcagctgtgagatgcttacgaaaatcccagtgctcaggaacctccgggagagagagaaacaggctgggaaggatgtcactctccgggacgagcctggcagccagcagccagctccaggccccatcaggagaccccaggatagaccacaaacaggttctcgaggagggtcccgtagtcccctgcagggggcggacacccaactagctgccactgtgtgtgtgggaaacctgcccttcactactcgtgtgcgtgagctgaagagagtcctccaggagctgggggtggtgcccctgaggcttacctggcaggggccacagcacagggcagttctacactatacagactcagctgctgcccagcaggccgcctctctcctacagggcctacgtctgggtgccaacgccttgagggtgtcactggggcagcagagggatatgtgacctggtgagtaacccccagcagaggagtagcgccctttctgctgctgggatgtgtctgccgtgtgacagtttgccgtgtgacagtttgccattggtgatgaccggctgccgtgtgaggcacctgctctgttcttcctgggaagggggccatgcttccctaatgggcagggatgacggcgagcagcccatctcgacacccaggatgctgtgagctctggcccattcctggtctggttcttgtgaaatccacgtgaggttctctgggtgcagaaggttcagggacagagcagtccagacaagaggtagacaatggagacctgtttcagggtgttcaaggagagcggaggcctgcagagaagaggccgtctcggggatcaagggcatgtggccgagcagggtgtccttacgaccactgggaacccacttagcagaacaggagcggggagggcgggcttctgcatccagagaacacaaacttggagctctgttcctggagacgctgcattgtgagcatgcgtgatggctgctacccaggcagcttagtcatgccatctctcaggtgaacatggcggctgcctgtgtgctttcagaagcacttgtgccgttgagcctaaggaaaaacaagcattaggtggagggtttgtcctgttcaaactgcacacctgggcagtttcttgtttttaccaatgggtcttccacagcgtctattagcattgtcctttgtcccaaaagggcaggcctcgctcatattaaaggaaaagagcttgtgacacg

SEQ ID NO: 61 - DNA, 2841 bp, Homo sapiens
gttggaggggctgagttgatgaaggcaggaatttagccatccggcttctcgccctctggcggctccaaacggcaggccgacttgctttcactacccgcatgctcctagatccacaccgagagtctccaggggggtgacttgtcgggtgcgggcttccagatgggggatgaggggccgggaagcccggtgcatctgctgtgcctcgcagcctctagcggggttcccctgttttgtcggagtagcagcggcggcgccccctcccgtcaacagcttcctttctctgtcatcggctctctcaatggagtccacatgttcggccagaatctggatgtgcaactgaactctgcaagaacggaagacacaaccgtagtgtggaagaacttccatgacagtatcactctcattgttctgtcatctgaggagggcacctctgagctgaggctggaaagaatgctccacatggtgtttggggccatggttctcattgtgggacttgaggagctgaccaatatccggaatgtagaaagactgaagaaagaattgagggccagctactgtctcatcgacagcttcctggggaactcagaactcatcggagacctgacccagtgtgtggactgtgtcattcctccagaggggtctgccatgcaggtattggggcagctatagaaataactgtgtccccacggttctgctcagccttcctttgttttctttctctggacattgtcctaactccctcttatcatcctggaatcaagttttacctggaaccttctcaccagggtacactgactgcttgcctcctgtcctgcagcttcggcctcacaagcaccctgccttcctgccttgcagggaaccctgtctaggcaaatccgcctcaccccattctaagacagggctcccaggctgtcttgggactctctctgtaggccagtccggccgacctccaacttacaactcacaaacacccaccagcctttgcctctgagtgctgggattaaaggcgtgctccaccatgcctgcccaatcttcctttgctaaagttatgcctctcctggcagcatgtcctgacctcagagcttccgtaggtcaggactgtgccttctcagcaagtttagatgctgttgttaaaactattggcatcccaggacctcctggtgcaggcctgtcaccccagctactctggaggctggggagagtatgcaaagtttaaggccggcctaggcagctcagtgaacacatgactcaaaaattcagtatgacttggcaggagcagccccataactgaaggagaaataaattttaagaagcaaacaaacaaacagataaaataagtgtatggagaattgagagtgctgactgatcaggaaggttaaagtattctcgttcacagagagggtgtgagacaggagcttggcagccttgcgtgctagggtgtctctggtctcatggctgctcttcccctccctgactagccgggaaggtccctccttgccctatgacatctagtctgaccttttcaccaggaaaccctttcagggtttgcagaggccactggcacagcctttgtcagcctgcttgtatcaggccgagtggtggcagcgacagagggctggtggcgcctaggcatgcctgaagctgtactactcccctggctggtggggtccttgccaccacaagctgctcgagactacccggtgtatctgccccacgggagccccacggtcccacacaggttgttgaccttgacgctgctgcgtggcctggagctgtgtctgctgtgcgggccgcgcccgccccttggccagctggacccacagttaatggaacgctggtggcagcctctactggagcccctccgggcctgcctgccactgggccctagggcgctacccgaagggttcccactacacagcgatattcttgggtaaggctcgatggttgggcccagggccgggaaggccatttgctctgccagttccccacagcctgatagtgctttcccccagtctgttgttactccacttggaactgcgacgctgcctctttactgtggagccctccaaggataaaggtgcgtgggaacgcagtgagtgggagggccgtgagccagaggccacctagtcaaggtgctccctcttctttagagccttcccctgagcagcgcaggcgcctccttagaaacttctacacgctggtggccaccacccacttcccgccagaaccagggcctgcagagaaacaagaggacacagtctacccagcccagatgcccagagcctgttatctcgtgctagggccaggcatggggtggcagctggtggctgtgcaactagggctccggctgctactattgctgctctcccctcacacccccacccacgggctgcgaagcctagccacccgcactctgcaagccctcaccccgctcctctgatcactcagggtgggcagtcaatagctggggtaatattagcgtctctctgtttattcgctcacaataatacacagcccctggatggggaaagggaggaagggggagggtgacagggaaggggggggagaggtacctacttcctcggcccctctcccctctgtgcttcgggggacaagggagggagggtgcttcccttgacccccccctagaatgtaaacagcagcagatgaacaaaaataaaatacaaaaggccagagggagctcccaggcgcccccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 62 - DNA, 4277 bp, Homo sapiens
gcggcggcctggaaccggttggctgggacctgacaccgcctgcaagctgcaggttgcttctataactggatcacgagtttgctgcacaaagtaccatgtctcgatcccggcagcccccacttgtaacaggcatctctccaaatgaaggaataccttggaccaaagtcacaattagaggggagaacctgggtactggtcccactgacctcataggcttgacaatttgtggacataattgcctcctcacggcagaatggatgtctgcgagtaaaattgtctgtcgagtgggacaagccaaaaatgacaaaggagacattattgtcacaactaagtcagggggtaaaggaacctcaactgtctcttttaagctactcaagcctgaaaaaataggcattttagatcagtctgcagtgtgggtggatgaaatgaattattatgatatgcggactgacaggaacaaagggattcctcccttgtctctccgtcctgcgaatcctcttggcattgaaattgagaaatgtaaacttcctcagaagaacttagaagtgttattccatggcatgagtgctgattttacaagtgagaatttttcagcagcctggtatctcatagagaatcactccaccaccagttttgagcagctcaagatggcggtcaccaacctcaaaaggcaggccaacaagaagagtgagggtagtctggcatatgtgaagggcggcctcagtaccttctttgaagctcaagatgctctctcagctatccatcaaaaactagaagcagatggaactgaaaaagtagaaggatccatgacacagaaattggagaatgtcctcaatagagcaagtaacaccgctgacacattgttccaagaagtattaggtcggaaagacaaggcagattccactaggaacgccctcaacgtacttcagcgatttaaatttctcttcaaccttcctctcaatattaaacggaatattcaaaagggggattatgatgtagttattaatgattatgaaaaagccaaatcgctctttgggaaaacggaggtgcaagttttcaagaaatattatgctgaagtagaagcaggaattgaagatctaagagagttacttctaaagaaattgctcgagactccatcaaccttacatgaccagaagcgctacataaggtatctttccgacctccatgcacctggtgaccctgcttggcagtgtattggagctcagcacaagtggaccctcaagctcatgcaagactgcaaagaaggccacatgaagagcctaaaaggtcacccaggcccgcacagccccatgctcgacctagacaacgatgtgcgcccctcggtgttgggccacctcagtcagacagcgtcactgaagagggggagcagctttcagtctggccgagatgacacatggaggtataagactccccacagagtggcatttgttgaaaaattaaccaagcttgtgttaagtcagctgcctaacttctggaaactctggatttcatatgttaatggaagtcttttcagtgagacggctgagaagtcaggccagagtgaaagatcaaagaatgtgaggcagagacaaaacgatttcaagaaaatgattcaagaagtaatgcactccctggtgaagctgatccggggagccctgcttccactcagcctccgagaaggggatgggaggcagtatggcggctgggaggtgcaggcagagctttcggggcagtggctcgcacacgttatccagaccataaggctaacatatgaatcactgaccgccctggagattcccaatgacatgttacagattatccaggacctaattttggatctccgaatacgttgcataatggtgacattgcagcatacggccgaagagataaagagattagctgaaaaagaagactgggttgttgataatgaaggactgacttccctaccatgtcagtttgaacagagcattgttcactccctgcagtccttgaagggtgttgtcgattgcaaacctggagaagccagcgtctttcaacagcctaaaacgcaggaggaggtttgccagttatgcatcaatatcatgcaggtttttatatactgcctggaacaactgagcaccaagcctgatgcagacatagatactactcatctttctgttgatgtttcttctcctgatttgtttggaagcatccatgaagacttcagtttgacttcagagcagaggcttttgatagtcctgagtaattgctgctatttagaacgtcacaccttcctaaacatagcagaacattttgaaaaacacaacttccagggaatagagaaaataacacaggttagcatggcatcgctgaaagaactggatcaacgactctttgaaaattacattgagctaaaagcagatcccattgttggatccttagaacctgggatctacgcaggctactttgattggaaggactgcctgcctccggcaggtgtcagaaactatttaaaagaagcattggtgaatataattgctgtgcatgcagaggtattcactatttccaaggaactggtgccacgggtcttggccagagtggtagaagcagtctctgaagagctgagccggctgatgcagtgcgtctcatccttcagcagaaatggagcactgcaggccagacttgaaatctgtgctctgagggacactgtagccatttacctgacctcggaaagcaggtctagttttaaacaggccctggaggccctgcctcagcttgcaagtggtgcggacaaaaagtcactggaagagctgctcaacaagttcaagagcagcatgcacttgcagctcacctgcttccaagctgcgtctccagcagtcatgaaaacataggctctgcaagaagagcatcagacatggcggtggagactgtcctcgtgctgtgcagcagagattacacgtcaagcactgcatttaacgtctcttttccccagcatggtggtgcagaaagatgcccatggatatttaatttcaatctatttaaactaccatagtttaaacatccaaataaacagtttttgttattgctagctttggtcctgaaaggctgtgtagcatactcttcaaaatgctgtgagtctggggatttctccatcatggtccagagcggcccatggtgtgtgtgtgtgcccttctcagtgcccctcttctaggcctactgttggcagtttccagcccaggtgagacatttgagaagagatttgaagtcacagtttttagagctttgtcctaaactttaaaataaccatttcatacaagtggttctaagctccccaccctgtttctgtaaagaaaaatatacagtctacacataaaaatgttaatatttggataaatttgtgatccacaagtcctcgtatgaactctgaatttcatagtctgactttcatttctcccagtgctaaacactgaagcttgtggttctcactcagccttaagctgatggatcactgcagtggtgtggggagggttcacaaggacctgtatgtggaggccctgtcacacccacaaggcctggggaaggcttgtggtcttcaatgcagcctaaagatgaagccaggctcctcggcaaggcagcactccctccccctctgccacttcccacgcggtgaaatgtcttgctccatagctggaaagaggctgctctggcatgggctgtgttttgtaaagatttgaggtgccccaagtgaccaggcactgagctgtgactcagtggtgagtggcttgcgcagtagagtcaggtcattgtgacctgcaggcagctttcacctcactccatccttccgtcagcctctgttgcacttcactcttcagactgatctgtggataatgtaggctcctcttctagagttgacaaaactaattccagagcacagcatatgtaccctgtggattatttaaggtaaatgagtgtatgtttatttatcacagtgactggaagtagaaattgtaatcctttgttgcaaaataagtatgtaccgcattgtctcctccctgtgtgctgttattatatctgacataaacaaactaaattccaaagcagaagtacagaaccatttgttattttaaacacctaataaatacagtgcacaaagccagtagttgtccttctctgtaagacccctatctttatttcaccatttttattagctctgtaattgcttatattcctgattttaaaatccacaataaatagtggagttctctcttttaaaaaaaaaaaaaaaaaaaaaaaaaaaa

SEQ ID NO: 63 - DNA, 3022 bp, Homo sapiens
  misc_feature (2699)..(2699): n is a, c, g, or t
ccgggagcagatggctggcatcaggggtggcctctcgatcagagtggagctagagatagaggaagcgccctaggctgggtcgccttgagcaactggtgaaactctgcgtctggtgccccgagtgtgtcatagtccagaagtgagtgagtggcactcggggctaacttctcagtgtgtctgttggcaggcgaagagtcgtatggcgcttgttctttcttcagaactgtcacgatgcatgtgtctgacgcttgtaggcgcctttagtgtttgctagttcttccctttcacgttttcaaatgtggcactgccgcgaacagaggctcaggcacatccgcctggtagtggaggacggagtgcatgagacaattgaggaaccaacacaagacagcagcccttcgaggtctttaggcagcttggaggagaacacatgagagaaagaatcccaagaggttttgttttctttgagaaggtatttctgtccagctgctccaatgacagagatacctgcacctttgtcctacttccagaatgcccagatgtctgaggacagccactccagcagcgccatccggagccagaatgacagccaagaacggcagcagcagcatgacaggcagagacttgacaaccctgagccaatatctaatgggcggccccagagtaactcaagacaggtggtggaacaagatgaggaggaagacgaagagctgacattgaaatatggagccaagcatgtcatcatgctctttgtccccgtgaccctctgcatggtcgtcgtcgtggccaccatcaaatcagtcagcttctatacccggaaggacggtcagctaatctacaccccattcacagaagacactgagactgtaggccaaagagccctgcactcgatcctgaatgcggccatcatgatcagtgtcattgtcattatgaccatcctcctggtggtcctgtataaatacaggtgctacaaggtcatccacgcctggcttattatttcatctctgttgttgctgttctttttttcgttcatttacttaggggaagtatttaagacctacaatgtcgccgtggactacgttacagtagcactcctaatctggaattttggtgtggtcgggatgattgccatccactggaaaggcccccttcgactgcagcaggcgtatctcattatgatcagtgccctcatggccctggtatttatcaagtacctccccgaatggaccgcatggctcatcttggctgtgatttcagtatatgatttggtggctgttttatgtcccaaaggcccacttcgtatgctggttgaaacagctcaggaaagaaatgagactctctttccagctcttatctattcctcaacaatggtgtggttggtgaatatggctgaaggagacccagaagcccaaaggagggtacccaagaaccccaagtataacacacaaagagcggagagagagacacaggacagtggttctgggaacgatgatggtggcttcagtgaggagtgggaggcccaaagagacagtcacctggggcctcatcgctccactcccgagtcaagagctgctgtccaggaactttctgggagcattctaacgagtgaagacccggaggaaagaggagtaaaacttggactgggagatttcattttctacagtgttctggttggtaaggcctcagcaaccgccagtggagactggaacacaaccatagcctgctttgtagccatactgatcggcctgtgccttacattactcctgctcgccattttcaagaaagcgttgccagccctccccatctccatcaccttcgggctcgtgttctacttcgccacggattaccttgtgcagcccttcatggaccaacttgcattccatcagttttatatctagcctttctgcagttagaacatggatgtttcttctttgattatcaaaaacacaaaaacagagagcaagcccgaggaggagactggtgactttcctgtgtcctcagctaacaaaggcaggactccagctggacttctgcagcttccttccgagtctccctagccacccgcactactggactgtggaaggaagcgtctacagagaacggtttccaacatccatcgctgcagcagacggtgtccctcagtgacttgagagacaaggacaaggaaatgtgctgggccaaggagctgccgtgctctgctagctttgacccgtgggcatggagatttacccgcactgtgaactctctaaaggttaaacaaagtgaggtgaaccaaacagagctgccatcttccacaccatgttggaaataaaacacgtcctagctgaacccttactgtccaggaagttccgtgtggaagtggcactgggccgggcctccctctcaggctcctttgctgcccacttgtaagtttaaataaggacaccgccctacacaaacctcaccctgtcacatcagtgactctgaccacttttgttctcaaactctctcactattatctgtggttgccgtttcttcccaaggccagcctggacgaatttggggttgctctatcctgagagttgtaacctcaacttccaaagtttatattttcttgaaatgatggatctattgctcaacagtccctgtcatccttaagtgacttctgggtttcccacaaattcctcacttttagacacactctaagcttacttctggcctgnatgcttcctctccctgtctctcccttgccccacagcggttccctgacagcagacaaggcagctctgggaggtagctagtatccaataacccaggggtttcctcatgtgatgcaaatactacgtgtccaaccaatcagtgctgtcaacgggctgccatagctccttcgatggcaaataggatgtgtgcccaaagaattaaagcgatgagtggctggtgaagcgctctctgtcctggctggttttttgtcaccgcaatccactgccaaaggcagaatttaaactaggggctgaaggatagctccgagtaatgtccaatgctggga

SEQ ID NO: 64 - DNA, 2079 bp, Homo sapiens
gtccgccgccgcctgctgggccggccgaggatgcagcgcagcgcctcggtggccaggctcgctctgctccagcgtgcttgctaacttccccggctccgtctctgcctgccggggtcgccccgtgtccctgcgccctccgggtctggttctctaagctctctgggcgctgcctccgggtcccttgcagcccgctcgcgagcccgcgctcccctcctgcgccccaccctcgtcctcgccatgctgcccttcggcctcgtggccgccctgctgctggccgcagggcctcggccgagcctgggcgacgaagccatccactgccccccttgctccgaggagaagctggcgcgctgccgcccccccgtgggttgcgaggagttggtgcgggagccaggctgcggttgttgcgccacttgcgccctgggcttggggatgccctgcggggtgtacaccccacgctgtggctcaggcatgcgctgctaccctccccggggggtggagaagcccctgcgtacattgatgcacgggcaaggcgtgtgcacggagctgtcggaaatcgaagccatccaggaaagcctgcagacctctgacaaggatgagagcgaacatcccaacaacagcttcaacccctgcagtgcccatgatcacaggtgcctgcagaagcatatggccaaaattagagatcggagcaagatgaagatcgtggggacacctcgggaggaaccccgtcctgtgccccagggttcctgccagagcgagctgcaccgggccctggagcggctggccgcctcacagagccgtacccacgaagacctcttcatcatccccattccaaactgtgaccgcaacggcaacttccaccccaaacagtgtcaccccgccctggacggacagcgtggcaagtgctggtgtgtggatcggaagacaggggtgaagcttcctgggggtttggaacccaagggggagctggactgccaccagctggctgacagcttccaagagtgagacctgccagcaggtgggggactcagtgccccctactaggcccctatgctctggggttgtagagttgacctggcttggagtctgagtcctggctgtctgcctctggccctgcccaaaatttccctttgtgtgtgtgtgcacacgcgtgcacatgctcgtgtgtgtgtgtttataggatgtgccctacaggataagcctgagccttctcgtgcccatgatgggcttagatacccccaagaggtctgagtgtagcaataggaattcccagtgtgctttcaaatcaatatggaattcattcattcattcattcattcatccacctgaaaacacgtattgaaaacatactatgtgccagctttagttttcagcgccgggaagcctcgttgtgatttcctccgattgtagaatgtgacgaggtgttctcaactcaaggcccacgggagaagagaaacctcattcttggagtgagagagaaagaaagtggtgaacattgaacgccctccctctccatcttggccagagcgtgggaaggtgtagagtagaggctcacggctcctgagacccacctctgctctgtgctgctgcgtagagctctctccttggggcctccagggcacaagcttgagggacagggcccatctttggtcagggtattcggtcatccgacaatgccttgcaggtagatctctgctgtgggaaaggaatggggtgaggaaggccggaaggacagggggcagggaggtgggctcatcactctgagcctgcaggagagaggaaagaatgtgagattggattgtgtgtgtctgaggaatggccccattctggtccctatttgtggttattggtcatgaagtcaccaggatgactctatctttctagttgcccaccacctgtggctttgcctccgtccccatatttccctacccttatacctcctatcccgccatctctacccccctgggcattttctggcttgcctggatagatttagagacctgcttgttggctataatgtcttttcattctttcttcttccttttttaaaaaaatcagaacaaaaccaaaaaaaaaaaaaaa

SEQ ID NO: 65 - DNA, 1728 bp, Homo sapiens
gagactcagaaagcgccgagagaagcaagatggcggacgtgctggatcttcacgaggcggggggcgaaaatttcgccatggatgaggatggggacgaaagcatccacaaactaaaagaaaaagcaaagaaacggaagggccgcggctttggctccgaggggtcccgagcgcggatgcgggaggattacgacagtgtggagcaggacggcgatgaacctggaccacagcgctctgttgaaggttggattctctttgtcactggagtccacgaagaagccactgaagaagatatccatgacaaattcgctgaatatggggaaataaaaaatattcaccttaatttggacaggcgcacgggatacttgaaggggtatactctagttgaatatgaaacatacaaagaggctcaggctgccatggaaggactaaatggtcaagatttgatggggcagccaatcagtgtggactggtgttttgttcgtggaccaccaaagggcaagaggagaggaggccgaagacgaagcaggagtccagaccggagacgccgttgataaatcctctgtcgactgggtggtctctctacaagtccatttggttatgctgccttggataaacagggctagggcggaccttgtgtttatatttaatttcttaccctatctacttggcattgcttttgagttttagaaaaaataaatgttccattttgttttctatgtttattactttactgttctggaaactgaacaactacagtggggcattgtgaatgattgtgaagatccttctgtctggcgtggcattctaagtttctagaaaggctcatggagactgttcagcatttctttgtcttagaagataagggtagggataatgagatttttcttgtggaattaaggaatgtattttgcataactcttgtgagcagatgttatattagtgtctcttgcttctctgtcagagcctgtgcagctggccttgaacttcaggggctttgtgatgcttagcctactctatgttgaatatggtgctgaactgtgaagtttattagttcttacaatgtaaaagaaacccagcttgatgggctggagagatggctcagtagttaagggcactgactgtccttccagaggtcctgagttcaattcccagcacccacatggtggctcaccatgggatcccatgccctctgaagatagctacaacatactcatataaataatttttttttttaagaaaaaaaacccagcttgggtactatgtaacaatatgcacacctttaatcccagcacttgggaagcaggtggatcttttggtttaaggccaacctggtctacaggagagttttaggatagccagggctgcacagagagatcctgtctcaagaaaaaaagacattgctatccctttatcatagggttttggttgttttttttttttgggggggggttttgttttttttttttaatttttctgagacagggtttctctgtgtagccctgactgtctcccctgtgcctcccaggtgctaggattaaaggcgtgcaccaccactgcccggcttttatcatagtttttagttgaaattattttttggtatgtctgtagcagctggtttgttgtttgtgtaggtccctatgtgacccacactggcctcagactgatgttcctgtctctctctccccagtgcataatggcaaatattaaacttaacgttttttcagatgc

SEQ ID NO: 66 - DNA, 4460 bp, Homo sapiens
aggcaggggctgcaaggaaaggatgccttcggaggcgcagaaccccaaagaagccagctcctgaattctgttactccatcagtggcttggaaagtcagatacatcttgagaactctgaaataatatggataaattcatagacaacatggatgtccgaattaaatcagaaagtggttcgatgcaggtattcaaacaggtcactggccctgttccgaccagagatccttctgcgagagcagacagaaggaatatgacttctccatcattcctggctgccagcccaatggagaacccagctctgtttaatgatatcaagatcgagcccccagaagagctcctggagagtgatttcaacatgccccaggtggaacctgttgatctctcctttcacaaaccgaaggctccgctccagcctgctagcatgctgcaagctccaatccgtcctcccaagccaccgactgcacctcaggccattatggtgcctacttcagctgacacagtcacttcggcagccattcctactgttctcactccgggctctatcctggcctcgtctcaggggaccggtggccagccgattttacatgtgatccacaccatcccctcagtcagtttgccaaataagatgagtggactaaagacaatcccactggtggtccagtctttgcctatggtctataccagtctgcctacagatgggagccctgcagccattactgtcccactcattggaggagacggcaaaagtgctggatcggtcaaagttgaccccgcctccatgtgcccactggagtttccaagtgatagtgatgagagtgcaattgaaagtggctcttcagccttacagagtctccagggatttcatcatgagccagcgacaatggtccacatgcagggagaagagtctcttgacttaaagagaagacggattcatcagtgtgactttgcaggatgcagcaaagtgtacaccaaaagctctcacctgaaagctcaccgcagaatccatacaggagagaagccttacaagtgcacctgggatggctgttcctggaaatttgctcgctcggatgagcttactcgccatttccgtaagcacacgggcatcaagccattccggtgcacagactgcaaccgcagtttttctcgatctgaccacttgtccctccaccgccggcgtcatgacaccatgtgaatatcacaggccacagcaggcacgctgcaatggcctcctgagtgtgtgctgaagttgtaactacgtttgcctctttgactacaacttgcccccatgattggggtccgaacagcctactgagccaggttaatgagactggaaaacaagttagctggtctcccatgcggctcttcatattccactttcacctcttcactgtccagacttcagtttttgcaacctcttcctgggttgaattcagtgcggcgcccatggctgccctccctgatccctcatgctacattatagacatttttcctacaataacaaacaaaacaggaatcaaactcaaggctgtgaacaaacatccgctgctctcccctcccccctggtttctcttgttttctaaagattttattcacatgtgtgtatatatatatacacatatatgtgtatgtatatatatatatatatatatatactgtaaccagtacttaatatcacttagcaatgtcctaaaatgagggttttttttcaaagttgtagctacaaggggcataacagtagagttggttgatcaaatttacatagaattgagtctgaaatacagagtctcatgctaaccgtgaaggttttgtagatccagttttacttgttcttaacagtttggtttacaggcagtgccagccctccttttgaagttcatcaagagcaatgaagggattccttgacctccttttcctcctcatgctcctttagaataaaaggttttatggcacaattccattctttttaggttaagtttggacaaatattcaaagcacagaaagatagatcagtagggagaagtattctaggaaaatacgtgggcagtgacaggcaagagcagacctgggagcaccctgtgaacaattacttggataaggtgttagaactgctgttaatcccaagcctcgttcagagggcagcctgttgaaacatcactttcgatgggctccactgtactgttgattcttttgtttacataaggcaaccattcagcgtattacctgatgagaaattgcacactgagcacgttatagagaggtgaggggttgcactaaaggaattttgtttacttcatgccattttgggcatttgttcttctctttctaatgggatggaagaggttttctttctgacactgacatatagaaagcatttttttggatgttttacaacttctattaatacccaagccatttattttatttagatataacaacaagatgcgtaatatacatttggttgcccaatcagactgttcataatattctctggggcataaaaccttctgttcctggccctggacctcatatctacctcttcgtggcatatttcctctcctggggaaaaaggagaggggctttgtattagtaggattggacacagcctctcgcctaaaggttttcttcatatgaaacctttatttcttggcagatattcaaaatgttctggcttgaacaatttaggttactacctcctgtggctgtactttctaactaaagcctagaattctaccatccagaaagggtcctgtctacttctacccatccctcacgttttacagatgaggcaaatacgggtcagattctttctccagataattccttgaggccttacagacctgtatacaccatctcccagggatcacactagtaaaccaaattaaagaaaaagaagttatgtgattggttgcaatatttgtccaattttatgttacacttcagaaaactttaaattcatgtatatttggtgacaaagtaagtagggtgtgcttatggtcatgaccacttaacaccaggcacacagtgtttcaagtcacattttttgataattctagatttgttttaatttaccttgtttcaagtttcaatgtctttgcagagcataaattctccataagttaaggcctacttgtacttttcaacgtggcatgtaggttttactgttactgctttggtaagggatctgtcctgactctgggtgtacaagttccaggctgtatactaattactaagcactaatcaatatctagggctagtctactaatggggaccacaatgtaaaacagattgaaaaatgtatattcctaccatatatgggcttaagcttataattgagtagttggtacaatactatagaacagagtccaaagggttatgtacttctgttccactttggatcctaaatttgttttccagagattggaattggcatgtgccccttagacttcaggatattgagacatatgtttattaatgagcactatgaaattatagtactcataagggtgatcagctgaggatcccagatactgatgttcctagcatctctgagcctttattcaagtatgtttctgcagagtcttaatagactttgcaaatgctttgtccattgtctttagccaagaaccaattctaaccatattctctttgctctttttgttcccaaatggcaaaaatctttccagttacaagcatagtctttacaaatgtgtcatgattaagagaacttcgcagagtaaatgaactcaaacagtacagtgcagaattactgcaccaaaagcacatacgtttgctacatagtgggatataaaggaaaccattccacttctcttggttaacatctgaggtttaactaagtgtaatgggcttacaacagccaaagactgttaggatgacatttgcctaggatatggaaattgtgtctggatggataatattcaggagtttattgtagtgaagtttctccctctgtagacatctggcagctgaacataattgttggtaattgccattgtaaagtgtaagaaatgtaaactggtttgatggattagtggagttcattatgtacaaacccacgaatgtttgtaaagagaatttctttttaaataaactttacatgttggaactctttttttgagattttcaggaaaatgtgaaggtagaacagagttcctgctgactctacattatatatttgtttggtatacttgttacaattagtgaaattataccgatacattaatattaactacaatcaatgctttcgtcagactaccttggcttttatctgctgttctttttttctgttttaataccgccttcaaaatacaacattgtattaataataacagctccttattctcctcgttgtgatagcttcttggagttctttgttctcaaggaccttggagtatgtttttcagttggggtgtattttatattcttctcatgaaaattttccc

SEQ ID NO: 67 - DNA, 2030 bp, Homo sapiens
gacggttcggcctggtgccgcgagcggccgttgccagggtgggggtcgctttgcggcatggcgatggcggagggcgagcggactgagtgtgctgagaccccccgggacgaacccccggccgatggcgctctgaagcgggcagaggagctcaagacacaggccaacgactacttcaaagccaaggactacgagaacgcgatcaagttctacagtcaggccatcgagttgaaccccggcaatgccatctactatggcaaccgcagcctggcctacctgcgcactgagtgctatggctatgcactgggcgacgccacacgggccatcgagcttgacaagaagtacatcaaaggctactaccgccgggcggccagcaacatggcactgggcaagttccgggctgccctgcgtgactacgagacggtggtgaaagtgaagcctaatgacaaggatgccaagatgaagtaccaggagtgcagcaagattgtgaagcagaaggcctttgagagggccattgcaggtgacgagcacagacgctctgtcgtggactctctggacattgaaagcatgaccattgaagatgagtacagcgggcccaagcttgaggatggcaaagtgacaatcaccttcatgaaagacctcatgcagtggtacaaggatcagaagaaactgcaccggaagtgcgcctaccagatcctagtacaggtgaaagaagtcctctgcaagctgagcacgctggtggagacgacgctgaaagagacagagaagattacagtgtgcggggacacccatggccagttctacgacctcctcaacatatttgagctcaacggtttaccctcagagaccaacccctatatatttaatggcgattttgtggaccgtggttccttctccgttgaagtgatcctcaccctcttcggctttaagctcctgtatccagatcatttccatctacttcgaggcaaccacgagacagacaacatgaaccagatctacgggttcgagggcgaggtgaaggccaagtacacagcccagatgtatgagctcttcagcgaggtgttcgagtggcttccgctggcgcagtgtatcaatggcaaagtgctgatcatgcacggaggcctattcagcgaagatggtgtcactctggatgacatccgaaagattgagcggaatcggcagcccccagactcaggtcccatgtgtgacctgctgtggtcagatccccagccacagaatgggcgctccgtcagcaagcgtggtgtgagttgccagtttgggcctgatgtcaccaaggccttcctggaggagaatcaactggactatatcatccgcagccatgaagtcaaagccgagggctacgaggtggcccatggtggccgctgtgtcactgtcttttctgcccccaactattgtgaccagatgggaaacaaagcctcctacatccacctccagggctccgacctgcggccccagttccaccaattcacagcagtgcctcaccccaatgtcaagcccatggcatacgccaacacgcttctgcagctaggaatgatgtgaggctgtcagcagcagtgagccgggacttgcccagctgaatggccccgccctcggcctgctgccccgccccagggcaacgttggacccccttttactttgtaaagtttgtatttattcccctttaggttgcagagggggtgggggcagcgtggggcagcagacgacaggctcctggtcagagaaaggagactggaggtagggcacagcctgggcatttggaggaaggccagcctgggtaggggtggggccaagaggaggtcttgccccttcacctgcatggctcctcccctggtaaagcaataggaccctgccataggaagaccccaaaagagggtcatcagggaggcctcctcctggtagcctcagggtggggcaaggctggggcccaccccccagctattttatgttctgtaattaactatgttaaaataaaataaagtcattattgtaagtcaaaaaaaaaaaaaaaaaaa

SEQ ID NO: 68 - DNA, 4305 bp, Homo sapiens
gcgtcgtgcttgcgggctggagcggttgctgccgtgagtccgagtgctttcgcgagccccgtcggcttcccggttcggtcggtcctcgacctcccgacaggctgctcccaggctgcgtctgggctggaaggtaagacttatttgtagtagataaatagccaccatggagaccaaggaccacaagaaacataggaagaagcacagtgggcctaaagctgagaagaaaaagaaacggcacctgcaagacctacagcttggagatgaagaggatgcccggaagagaaaccccaaagctttcgcagtgcagtcagctgtgcggatggcgaggtcctttcacaggactcaggacttaaagacgaaaaagcatcacattccagtggttgacagaactccactggagcccccaccaatagtggtagtggtgatgggacctccaaaagttggaaagagcactttgatacgatgcctgattcggaattttaccagacagaaattgagtgagatcagaggccctgtgacaattgtgtcaggtaaaaagcgcagacttacaattattgagtgtgggtgtgacatcaatgtaatgattgatctagctaaagtagcagatctggtgctaatgcttatagatgccagctttgggtttgagatggagacatttgagtttctaaacatctgtcaagtacatggctttcctaaaattatgggagttctcacccatttggactccttcaagcataacaagcaacttaagaagacaaagaagcgtttaaagcacagattctggacagaagtctacccgggcgccaagctattctacctttctggaatggtgcatggagagtatcagaaccaagagattcacaacctgggccggtttattacagttatgaagtttaggcctctcacatggcagacttctcatccttacatcttggcagacaggatggaagacttgacaaatcctgaggctatccgcacaaacgtcaagtgtgaccgaaaggtgtctctttatggttatttacgaggggcctacctgaaaaataacagccagatccacatgccaggcgtgggggattttgtcgccagtgatgtcagttttctcccagacccctgcgctcttccggaacagcagaagaagcgctgtttgaatgagaaggagaaactggtttacgcgcctctctctggggttgggggtgtgctgtacgacaaagatgctgtctatgtcgacctaggtggcagccatggcttccaggcagcggaagaaacaggccctacgcatgaactggtccagagcctcatctccacccatgccagtatcgatgctaagatggcttcaagtagagtgacacttttctctgattccaaacctctggggtcagaagacatagataatcaagggctgtggatgccgaaagaggaaaagcaggtggatgtggaaacgggccgagtgcgtcggaaagccatttttggagatacagaagatgagtctggagatgaagagagtgaagatgaagaagacatgtctgaagctgacgggatggaacctggctctagtgatgatgaggcggaagaggcagaggaagaaggtgctgagagcagcagcggtaactacatgatgggtaggggacgcaaacgaccaaagcttgaggagcaggaggaagacagtgaggcagactcgccagcgtttgctgatagcgatgatgaccttgagaggagctctgggggcgaggaggaggccgaggaggcggatgaaagcagtgctgacggtgcctccacaggggagagggacgctggggaatgcggctctgttggagaagctagtgaggagagcccgtcagcagcccaccagtggaggccccatgcggatgcacagaagccctccccggtgagaaaagcagccctcaccacttctgactctggccactgtacagccgaggaggcgtttccatctgaggatgagtctgaggactctgagctcagtgcagaggaagaagactccgaccacagagaggttagggggaagcttgcacaccctctgcgcgacagcggacagaaacctgcctcggagagcctggttgatgagaccagcgacattgaaagtttactcaaagaggaggaagagtacaaggaggacaatagcagctctgtagaaacgtcaggtgctctgaagtggaaggaagacctttccagaaaagcagcagaggcctttctcaggcagcagcaagcagctccgaatcttcgaaagctgatttatgggacagtgacagaggataatgaagatgaagatggagaccccgcagaagagcttggagggctgtttcatgtcagccagccagatagagggtgcaagcacaaggctgactctctggactgctccagattccctgtggaggctcctcacgactgggatttagaggaggttatgaacagtatcagagactgctttgtgactgggaaatgggaagatgataaagatgcagccaagatcttagcagaggaagaggagctctatggtgactttgaagacctagaaacgggggacgtgcacaaaggaaaaccaggcccggatactcagagtgaagacatagaggaagaagaagtgaaggaagaaactgacccaagtgaggaggaaagtgctaggaaaaagcatctggataagaagagaaagctgaaggagctgtttgatgcagagtatgatgaaggagaaagcacctactttgatgatcttaagggggaaatgcagagacaagcgcagcttaaccaggctgaatttgaagatcaagaagatgaaaccagagttcagtatgaggggtttcgacctggaatgtatgttcgaattgagatcgaaaacatcccttgtgaatttgtgcagaactttgacccacactacccaattattctgggtggtttgggcaatagcgagggcactgtggggtatgtgcagatgcgcatcaagaaacatcgttggtataagaaaatcctcaagtctcgagaccccattattttttctgtaggatggaggagatttcagaccatcccattgtattacattgaagaccacaatggaagacaaaggcttctaaaatacaccccacaacacatgcattgtggagcaactttttggggtcctatcactccacaaggaactggatttttggcgatacagtctgtcagtggtgtaatgcctgagtttcggattgctgccactggagttgtccttgacctggataaatccataaaaattgtgaagaaattaaaactaactggttttccttttaaaatattcaagaatacttcatttattaagggaatgtttaattctgcattggaagtagccaaatttgaaggtgctgtcattcgaactgtcagtggaataaggggacagatcaagaaagcactccgggctcccgaaggagccttccgcgctagctttgaggacaagttgctaatgagtgatattgttttcatgcgaacttggtaccctgtctccattccggccttctacaatccagtaacatctttgttgaaaccagtgggtgagaaggatacctggtcagggatgcggacaacacatcagctgaggcttgcccatggaatcaagttaaaggcaaacaaggattccttgtataagccaatcctgagacaaaagaaacattttaattcactgcacattcccaaagccttgcagaaggctctgccatttaagaacaaacccaagactcaagcaaaggcaggcaaagtgccgagggacaggcagagaccggccgtcatacgtgaacctcatgagaggaaggtcctggctctgctagatgctctgagcacaatacacagtcagaaaatgaaaaaggccaaagagcagagacgcctgcacaacaaggagcatgtgaagatgaagcagaaggaggaagaggacaagctgaggcggcagaaggacctgaggaagaagctcttccggatgcagggtcagaaggaaaaacgaagtcagaagtctagtctgagaggagctgaggatacctgaatggagggggggtcttccagagctgcagccgtgatggcttcagtgcttgtgacaagtcatgacgagagcttgtggagactctgggatgtgtggtcccgccgtgcctggaggaactgtagaaagcctgctggtggcatccgtgctcattcacggcatccagcaagtcacagttcaaggctgggaaacttggggctttgtgaattcactcattggttcttcctgacttggtttattttgtgctataaaaatattaaactattttcccattaaaaaaaaaaaaaaaaaaa

SEQ ID NO: 69 - DNA, 2695 bp, Homo sapiens
caagactggtcagcggctccctaaccccgggctcgcaggctgatgaccctgggagacaggggcctcgaggggccgcaaactagcgcgccgcgggctgagcccgagaggccaggctggggggagggaggcccggagggagggagtgaaggaaggaggagggctggcgagctggagccggcaggcggcgggagcccgggcgagcctcgtcgggagtgcgtctcctcggcagagcaggacgcagccagagcttgtccaggtgactgagctccacccctgtgcaccaagacacccaccccaccccccactggtcaagcctcgctggagaaagagaccgggaagtcccagcatgagaatcagagctgcccagccagcttcatagatggggaagcagcctgcttgcctcccccatcaattacagggataactgagaaaaagccccacccaatatggaggctgaggactaccgcctctattaattccctgccttctcccctgccctgcccacctggccttagctgggtcactcatgcaatgctgagaagtaagcaagacttggggtcagcttgcctggctaccaggcaaggactgtgggcatcatgggggtgtgtgtgagcggggctccagggtgagacctagcccccgcccccaggaattcaagggggtgggggtggggccgaggatagaatggctcggggagggttgggggcagaagaggcctccctacgctcaaacgcattgtcctggctggcctgtgggctcctggccctgctggccaacgcttggatcatccttagcatctcagccaaacagcagaaacacaagccactggagttactgctctgcttcctggccggtactcatatactcatggcggctgtgccccttaccacctttgctgtggtgcagctccggcgccaggcttcctctgactatgactggaacgagagcatctgcaaggtctttgtgtccacctactacacgctggccctggccacctgcttcacggtcgcctcgctctcctaccatcgcatgtggatggtgcgatggcccgttaactaccgcctcagcaacgccaagaagcaggcgctgcatgccgtcatgggcatctggatggtcagcttcatcctctccacgctgccctccatcggctggcacaacaacggggagcgctactatgcccgaggctgccagttcatagtctccaagatcggcctcggttttggtgtctgcttcagcctcctgcttcttgggggcattgtcatggggttggtctgtgtggctatcacattctatcagacactgtgggcccggccccggagggctcggcaggcccggagagcggggggcagtgtgggcaccaaagcaggggggctgggggggttgggtacccggccagcttttgaggtgccagccattgtggtggaggatactcgagggaagcggcggtcctcgctggatggctctgaatctgccaagacatccttgcaggtcaccaacttggtcagcgccatcgtctttctctatgactcactcacgggggtgcccattttggtggtgagcttcttctccttgaagtcagactcagctccgccatggatggtgttggcagtgctgtggtgctccatggcacagacactgctcctgccctccttcatctggtcctgtgagcgctaccgtgcagatgtgcgcacagtgtgggagcagtgtgtggcgatcatgtccgaggatgacggtgatgacgatggcacctgtgacgactacacagatggccgagtgtgcaaaattcgctttgatgctaacggggccacagggtcagggagtcgggaccccacccaggtgaagctgctgcctgggcggcacatgctcttcccaccgctggagagagtacactacttacaggtccctttgtcccgacgattatcccacgatgagaccaacatcttttctactcctcgggcaccaggctccttcctgcacaagtggtcctcatcggatgatatccggatcctcccagcccagagccgagccctcggaggccctccagaatacctgggacagagacacaggctggaggatgaggaggatgaggaagaggctgaaggcgggggcttggccagccttcgacagtttctagaaagtggggttctggggtcgggtgggggacccccacgaggtcccggttttttccgggaggagatcaccaccttcatcgatgagacacctctgccttctccaactgcgtcaccaggaccctctcctcgtaggcctagaccactgggtttttcacctcgacggctctcccttgggtctccggatagccgagccgttggacttcctttggggttaagtgcaggaagacgctgttccctgaccgggagcgaagggagttcaagggcttggggaagaccctgggggccaggtaaccccatcttcccccagttgaccctgtgaggccgcataggcctgcttcactggaggggggggggaggcacgagcgagtgagtatgcctcatcctggccctgagtctatggcagctgcctccagggagatgggggggggggggcctacatccgggctctagagccccagctctctcccatccaagtgaccagatacccaacgcacccgtcttcacccctagcaaaatgtattaaaaggctgagctgccgtggaaaaaaaaaaaaaaaaaaaaaa

SEQ ID NO: 70 - DNA, 2452 bp, Homo sapiens
agcatggagggtctgtacttgagtctaactaggtcagatgctggggatctttaaagctgattcctgcaataatgccaggcagtctggttttatgacaaggttttgacctggacttctggaccagatgcctgggaacagatagtgtcaagcctttctgaatctgtcgcagatcatttatgaaaattgattttagccaatcataaattgtttgctactgccttccaaggtaagtgaaaaacgattggactgagtctatgttatttgacctcaggtgatattcccaaagagaaatgacagaagcttatctttgaatgcaaacaagaaatgtgaacctcctgatcagtgcagcactgagtgtgttactaatctatgtatccctaccccttccctctctctcttttaccctcctccctcctctcttctccctcttctatcctaccttcactttcaagaaactagagagccacctttttataagaaagttaagtgtcttaaaatttttaggtagaaaattttgtacgttttcatccgtgtggctataaacaccaacataggactctatttgcatattaaaacaaacactgcagacactgatctaaccttccgcagtgttcaactggaatttgttcagatgtcagccatgatgctgattggtaatgtatcagtggaaaacattaaggaaattggggtgtgacctaatggacagtaattacaaagctattacaaatctcattgcataagtaatgtctggaaactttgtgctctctcaaataaacctcgacagccagacgagtgcttcgcaatcacacagtggcatcataatcccgaaattgactgttctgtgctaattcacgctgatgacctcttttaaaattatttttaatttggcttcataagaacaattccaaagtagaagtgtgttgtcttttctggcattgtttttctttttccaatttgaaatgcaaccagctgtttctgtgtaataaaaactgaaccatgaaatacaatggctctttcaagcttcaacctgcaggcagctcacaattagcaaccttgtgttatactggaaatagccaattaaaaaaatgctgaacattctacattacaaaatatcaaatggcatgcaatcaaggtccctttaaaaggcaagctgctctttctctataaatttgccaataaaacgattaggatattattctaaaatcagtatgtaaaaatttccaaaagtcttttttaaaaaatctgtcagtgtcagtgttgaccagttccttctatgaggcttcacaaatgccagacatgatggcatttccattttgtgggtatctgccatatcaccaccttcaactaacatatccaccatgcaactgtattttccaggaggcttctggaaagattgcctgcatacatgaggatgatgtccagctgagttcagtgtctagagaatggactctgtatataggctgctcagtggtgacacgcggtgggcccacaataatttctgctaagtgggaattctgttggctctttggcagtactcaggaaactggggtacagtcaggtttccagagccaaatggggataagctggactctgaccagtacatacactttgacaatgtgtaaggaagtgttactttgatgtgataacacagatcacttttttttatttaagaaaatgcatgtccaggggtagccaggatttatgcatctacacctataacatttgcatcttagctgctcaccaactcaccaatgtggaaaactaagtcagctcagatctgaccatagaaactcaagtcttactctgctccgaagaaaagtgctctaggctagactagcatgtctaccataggaaccactggcttaatgtggctcatgaccgcccgatgtaaacatgcttaaactgttaatttagaaacagtataaaatagattcaaattatcttattaattttaattttgatgtaataatatgtgtatactggattaaaatatagtattaattccatattttctttttagaatgtagctaatagaaaatgtaaagatacatgcatggttatgggagtggtggcatatgcttataaccccagatccagggaaagatgaggcaggaagatcactgtgtgagtctgaggtcagcctagattacaaagtgaattcaaggccagcctgcaattcataataaaaccttgtctgagaattgtttttacatgtgtaccttattatactttttaatataaacacttatttcagaagataactttgtaaccattctattctaatctctcatttatgtgctgttgtggcaagagggctaacagtttgagaaaccactgtaagcacaggtggactttctaaataggagaaaaaaatttgcagactgtcctctgtggtgaaagaaccccgttaagctatatgtctgatcttgaaataaaaaccaataatgaatgg

SEQ ID NO: 71 - DNA, 1438 bp, Homo sapiens
gagtctgagttttcccgttcaggtactatactagtccaccagtgaccccggacagtaacaaagcattacagcccgaacccacctcccgccaccatcgtaggttctgtacctcacttgggaatcagtaaaccatggcatctaagaaatttgccgttaaatgtgggaacttcgcagtcctggtggatcttcatgtattgccacaaggttcaaacagagactccagttggttttctgaacagaagaaagaggaagtttgtttgctgttaaaagaaaccattgattccagagttaaggagtatgtgggcatttataaacagcgcaagccatcaagtgcagaattcacaagatccagtcccttgtccttaaaaggttatggctttcagatcacagcttatttcctcaagagagggatacaccttcactgtatccagaactcccagaatactgaacttcgtgtatttcctgaaagatttgtggtgtgtgttagtcagcttgcatttggtcatgatatttgggcaaaccagaatgaaaaatcgacaaaaaaagccctccatggagtgtctgattattttcctgagtgtgcagagagttcaccgtctcctggtacaaagctcaagaggaacgctctgaaggaaattgtgagaagaactaaatcaaaaggcactgatgtgagcaaaccacagcccagcggggacctcgtgggaagatctagtgactcagtaattactgtggtaccatggaggagggatgcctcagccattctcctgtcagaatctgtgggtcaagcacaagatgacataagagcagccaagagccaccaggagcttcctgttcaaaagctggaaaatgtttctcagactcagccaggagacactcgcagccagcagcaacttcatcctggggagtggttgaagacggggcttctaagcaggagccctgcgtataactatgagtcagcatcaccaggtccaaagcaaagtctgagagcagccaagacacagcagaaacacaggaattgtggctctgtggaagactgtgaccaccgcaggagagtttctcttggaaatgaaggattagtcccggaggacgcggatagggaaaggagcacagctgtcagggttttaccagccttagagttgtcagatcctgggttgcttttgaaacaagatttggcaaaagccaaggctaaggaagagctacatgctttggaaaatctctcctccagacatcttgtgacaaacaacccggggcaggcacagcagagcgactcagctgcaattactgaacaattagctacagatcagggcggccccagcaagaaaagaaagaaactacaaagttacaacagaggatgtagtggcaagaaaaactgaggatacagtacctggggtattggaaggaattaggacaattactttaaataaataaatttaggagc

SEQ ID NO: 72 - DNA, 2700 bp, Homo sapiens
  misc_feature (664)..(664): n is a, c, g, or t
tcctgggagaatccgtcaccgggaaagagtagaaaccacctggtcgaggcgcagaagtggctccggagggagccggcgtaaggtctgctgcctcgtgtccgctaagggcgcggcgatccgggatccgccggccggagcccaagcgcagccgggagcttgcgggtggcgaagccaggcgcaccgccttctgggtgtctgctgagcggttccccgcgtcccgcggggtcctgcagccccgaccccccggctggaagccacggagaaccgctgccgaggactgcggaccaacatcgtccctcccgaaagcggattctgctatgacagctgggctctccaaggggttaacctgggtgtcctccgtaaagttgtcgccgagccgggagcccgtttaggggtcgcagcgccgcggctcggggggcggccgagtggccggcggcggccgtggctcggcggaacccgcgcggccggggggcttctgggggtgtgcgcccccagccggctgccctcgtggatgcctcccggcggcggcgggcccatgaaagactgcgagtacagtcagatcagcacccccagctcctctcccatggaggcgccccaccagaaaaagaatatcgctgcccggaggaaatgggaggtgttcccaggaagaaacaagttcttttgncaacgggaggatcatgatggcccggcagaccggcgtcttctacctgactctcattctcatcctggtcactagtggactcttcttcgccttcgagcagctgtcggtacctggcagagaagatcacccctgccatccccgtggtcggtggcatcctgttcttctttgtgatggggacactgcttcggaccagcttcagtgacccgggagtcctccctcgagccactcctgatgaagccgctgatctggaaaggcaaatagatatagccaacggcaccagttcaggggggtatcgcccgcctcccagaaccaaagaagtcgtcatcaatggccagaccgtgaaactaaagtactgttttacctgcaagattttccgcccccctcgagcctcccactgcagcctgtgtgataactgcgtagaacagtttgatcaccactgtccctgggtaggcaactgtgtggggaaaagaaactacagatttttttatatgtttattttatccctgtcctttctgacagtctttatatttgcattcgttatcacacacgtcatccatcgttcacagcaaaagggattcctcgatgcccttaaggacagtccagcgagtgtgctagaggctgtaatatgcttcttctctgtctggtccatcattggcctttcaggtttccacacgtgcctgatcagctccaaccagaccacaaacgaagacatcaaaggctcttggtccaataaaagaggtaaagaaaattacaatccctacagctatggaaacatcttcacaaattgctgtgtcgccctatgtgggcccatctctcccagcctaattgacagaagaggctatgtccagcccgacacaccacagccagcagcaccatcaaatgggatcactatgtatggggccacacagtcgcagagtgacatgtgcgaccaagaccagtgcattcagagcaccaaattcgttttgcaggccgcagccacgcccctgcttcagagcgagcccagcctcaccagcgaagagctgcatatgcctgggaaacccggcctgggcaccccatgtgctagcctgacgctgggccagcccacaccaccctcctccatgcccaaccttgccacggaggccacactctcagacattatgcccctgaaggatgagcatgggggccatcagtttctgaccccagatgaggcaccctcaccgccaaggatgctgggggcaggcagccctctagcacatagccgtaccatgcacatgttgggtctggccagccaggactccctgcacgaagactccgtgaggggvttggtaaagctcagctccgtgtgacctgcatggccagccctggggaccacaggtgacaaacaaaaacaaattactggacatagccaagaggacgttttgtttggaatctgcagagtggaccgggaagaggagactccctcaatgggaggagcctggaattgaaagaggcagcgtggatgcagcgtgcagcgccctctgtcggctgcttttgggacagacacgaggaaaattttcgacactggtggccagactgaaattggacctaatgttacagctactaattgaaatccacccgccgttccatttgttcattaaaacaaaacgaaacaaaagttccaaagggaaaagccggccagacatgggattgcatgccacactatagtctgtgttacagcagtggtattccagaagaaaagatgtctctctgttttccttctttttaatcttgtgatactttccaatgtgacagaccgaacccggtgggtaatgccatccttcgcgacttactggacttagctgtatgaggatgggcgtttcctgatgaagagggttactgggggcatcccctgttgcagacggcacctggggtctatcctgccctggacagacattttaaccctctgtcaatgctcagatggatgtcatattgtgccaccatggggcagtcctcgcctcctgtgtgacaatgaccttcctatggacagccaataaaatgactcctctgttaaaaaaaaaaaa

SEQ ID NO: 73 - DNA, 3485 bp, Homo sapiens
tagacttcggccgtggtggccaggcgctccccacacattgtatacgcttgcagcttcaggaagctgctcctcaggctgtgcccatcctgtttggcagcccagctaccatggaggccccagaagtccctgtgggctccctgattgactttgagactgaaccacctacctcccctcctttggaagcagcaccgtcaacgctccaggacccagatggctccctgggtgatggtgcatctgagagtgagaccaccgagtccgcagacagtgagaacgacatgggcgagtcaccttcacacccatcctgggaccaggaccgacgctcttcctccaatgagtccttctcctccaatcagagtgctgattccgccccagatgaggagaccctggcacttcgagagtttatgcgcagctacgtagagaagatcttctctggaggggaggacttggaccaggaagagaaagccaagtttggggagtactgcagcggagaagacgggaaaggccgggaatggttcgcacgctttgtgagcgcacagcgctgcaagtccaagtgtgtgtcggagccaaccttgtaccgcctggtgcagtccttcgcggtagtgctgtttgaatgccaccagatggacgacttcgggcctgccaagaacctcatgaccatgtgcttcacttactaccatctgggcaagccccagctgcccccgacagagcccagggagaagccagcaggcagtattgactcctacctgaagtcagccaacagctggctggcagagaagaaggacattgccgagcggctgctgaagaacacagagaacatgaaaggctttttcggggggctggagaccaagctgaaaggaccgctggtcaggaaaaatgaggaagatgagaacaaacctaaggacagacagacgaagactgtgaccatgatcagccctgaggatgagcagaagggggagaaggtttacctgtacacgcacctgaggcagcagcccatctggcacacgctgaggttctggaatgctgccttttttgatgctgtccattgtgagaggcggaagcgatcccccaccaccagaggggatgctggagagcaggaggagaagagggagaaatggtgccacatgacccaggaagaacgggatgacagcctgcgcttcaacgagaacatcaccttcgggcagctgggcacgttcacccacaacatgctggcctttggcttgaacaagaagctatgcagcgacttcctgaagaagcaggctgtgattggcaatctagatgaagagcaatacaagctgctaagtgaccacatcgagcaaatggccacagagtaggagatcagcacgtgctcgtctcctaggatctgtcaccccagtgatgaccctgcatgacaccagcagcacccagagccgctccttgctgccctagaactagcagttaaaagactccagaagcctgtctcccctgcctgtgtctgctctccctctatgtctatggtgtcccctgggttacacagctaaacgtgagggaccatctaccccccacctatcctcgagcaccaggcttgtgagatgagacagagggtggaggaagccatgagggatggtccccacggatcctgccgagagccacggacatgtggttacagaggcgcaggaggggatccacacagctgcaacgtgaggacagaatcagtgctgtgaatgacagcctgccactccctgccacaggactccctgtgagctgtacagaggaccatggagccttcctgaaaggtctcggccatctgttcttctccacctcctcatgggataggaactgaaagagaactcaagggttgtcactttcctagagggtgtgagcgttctgccagccacaaccatgtctcagactcaagagctccgtcaacagagcctcggaactggccactcaatacagtctctccctcaatagcacagaaggtccttgagctcccaggccgcagcaacacatgcagtgggacgcaggaagcgtgggcgagcattctcctcggcagctctgggggggacagagctaagagcacacagaatggatctgactgagggaggagaccacgggaaagcaaggttgcctggtagctccagcccacagtcctccccttctgaaagacccacagcgatctgcctcctcagcatccagcgtgctggctccagagctgctgttcccatccacgccagtggcctcggtcccttctgtgccaggcattctggtcactgaccttgaactaggcctgcaggctctcctgtcaccccgcatcacctcacccttctctgtccctcagccttcggtcacatttagtcatggtctgcctgtgagcgcagtgagttctctcctgacagctgtgagctcagctggtgtgggtgagcctttcctgcaggaagagcctcccaagggatggccaaagaagagcaggataggcaaagctgtggaatgtacatttcctggatgtcaggggtgaccaaaacagtacatcagttcctgagatattcaagcaaatgcccccgatagcatagtgtgtgccaaccggtacacctgactcagcattaggatcctataaccatgcctgtccctgctctgtgttttagggaagtgacataaagtgaggtcgcccttgctaagtcctgctgtcctctctagagccttcctcagagctgtgcaagcatgggagcctctccttgctcctcccagtcctgcactggcatgagttcgggtgtaggaggacatgactccaaggtgctgggagatgcaccagtcttgcgcagcagcacctgtggtctggcctctgccttgccctcatgcagccatgagctgccttaaggggaagagtgccaatggtcttcgtgggcttcagaagtgactttgagactgttcctgctgaccttttgagatagtcacttaaggaaggatggctctctctcatctgtctcttgttccttattgttgcagagctagctccacagagtcagaaagttctgacccacattagacccgtgtgcgctgctaaggccatggtaaccctctggggacaggcagccattgatgggagcaaagcttgcaggtgcctggaggtggtcccaccattgcctgcagatgtccttcctctgttcacagttaggtttagtgagcatttcatgtcagggagcctctggccagtgtctctgggaggtcactaatctacccctgaggtacccactctttctgtcgtagctttcttctagggccccaacaagcaagcataaaccaggcttgctggccagcactccttggctcccctgccgaaccacagacagacagttcagattaccaggataatcccaagcagtatctccagtctcggctggtagtggcccagatctatgcaggcccattagcacacattcaaaccttccggggctgtgaccttcaaagctgtgtaaatcagtgtcaagcacattatagtgtttgccctc

SEQ ID NO: 74 - DNA, 3685 bp, Homo sapiens
gcggccgcgaattcggcacgaggggcggcgcgaggccggccgggagggaacagcctgctttctccggagtccggagcagccaaggactgagctgagcaagcggagcgcggtgcctgcgggaagccgggacagcagcagcctcaccgtgccatgcgggcctcaggattgtgccaatgaggacctaaggtgcctgcccgccaaccatcccagcctgcagtgaccatggccctccgcaagaggagtcctcatggcctgggcttcctttgctgctttggaggcagtgaccttcccgagatcgacctccgggacagccacccactgcagtatctggagttctctggtcccatcccgaatcctgaagagctcaatgtccgctttgcagagctggtggatgaattggacctcacagataaaaaccgagaggctgtgtttgcactaccacccgagaagaaatggcagatctactgcagcaagaggaaggagcaggaggatcccaacaagctggcaaccagttggcctgagtactacattgatcgtatcaatgccatggccgccatgcagaatctgtatgaaactgaagatgaggagacagacaagaggaaccaagttgtagaagacctgaagacagccctccggacacaacccatgaggtttgtgacccgcttcatcgatctggaaggcctgacctgtctgttgaatttcctccggggcatggaccacaccacctgtgaaagccgcatccacacatctctcattggctgcatcaaggctctgatgaacaactctcagggccgggcacacgtcctggcacaacctgaggccatcagcatcatcgctcagagcctgcgcacagagaacagcaagaccaaagttgcggtgctggagatcctgggtgcagtgtgccttgtacccggaggccacaagaaggttctacaggccatgctacactatcaggcgtatgcagccgagcgcactcgtttccagactttgctgaatgaactggatcgaagtttaggccgctaccgggatgaggtaaacctgaagacagccatcatgtccttcatcaatgctgtcctcaatgctggcgctggagaggataacctggaattccgcctgcatctccggtatgagtttctgatgctggggatccagccagtgattgataaactccggcaacacgagaatgccatcctggacaagcatttagacttctttgagatggtccggaatgaggatgacctggagctagccaggaggtttgacatggtccacattgacacaaaaagtgcctcccagatgtttgagctgatccacaagaagctgaagcacacggaagcctatccctgcctgctctctgtcctgcaccactgcctgcagatgccctacaagcggaatggtggctacttccagcagtggcagctcctggaccgaatccttcaacagattgtcctccaggatgagcgaggggtggaccctgacctggccccactggaaaactttaatgtcaagaacatcgttaacatgctcatcaatgagaatgaggtgaagcagtggagggaccaggcagagaagtttcggaaagaacacatggaactcatgagccggctggagagaaaagagcgggaatgtgagacaaagacactggagaaggaggagatgatgcggacgctcaacaagatgaaggacaaactggcccgcgagtcccaggagctgcgccaagctaggggacaagtggcagagctggtagcccgacataatgagagctcaaccggcccggtgtcctccccacctccccctggaggtccactcaccttgtcttcctccaggacaaccaatgatctgcctccacccccaccacctctcccctttgatagctgcccacccccacccgcaccacccctccctcccggagggcctcccatccccccaggtgccccaccctgcttcagctcaggcccacccccatctcatgaccccttctccagcaatgaagccccactcaggaaaaagcgcattccccagccttcacacccactgaagtccttcaactgggtcaagctgaatgaggagcgtgtttctggcaccgtgtggaatgaaattgatgactcgcaggtatttcggattctggacctggaagactttgaaaaaatgttctcggcatatcagaggcaccaggcttgcatgcaggaggggccccagagggagagaggaaatgtgagagacggaggggcggcgagcagacctctgccagctgtggaggcgagtgcccacagaactgagaaggcttctaggtctatggtatcagctacaggtgccaagaaagaactgggctccacggaggacatctacataacctcccgcaaggttaaagagctgtcagtcatcgatggccggagggcccagaactgtatcattcttctctccaagctgaagctttctaatgatgaaatacggcaggccatcttgcggatggatgaacaagaagacctcgctaaggacatgctggagcagcttctcaagttcatccccgagaagagcgacattgatctcctagaagagcacaaacatgagattgagaggatggcgcgtgcagatcgcttcctctatgaaatgagcaggatcgaccattaccagcaacggctacaggccctgttcttcaagaagaagttccaggaacgcctggctgaggccaagcccaaagtagaagccatcctgctggcctcccgggagctgactctcagccagcgcctgaagcaaatgctggaggtggtcctagccatcggcaacttcatgaacaaggggcagcgtggcggtgcctatggcttccgggtagccagtctcaacaagatcgctgacaccaagtccagcattgacaggaacatctctctgcttcattacctgatcatgatcctggagaagcacttcccagatatcctgaacatgccctcagagctaaagcacctatcagaagctgccaaagtcaacctggcagagctggagaaggaggtgagcattctcaggagaggcctccgagcagtggaagtggagttggaatatcagagacaccaggcacgagaccccaatgacaagttcgtccctgtcatgagtgacttcatcacagtgtctagcttcagcttctcagagctggaggaccagctgaatgaggccagggacaagtttgccaaggcactgacacatttccgagagcaggatagtaagatgcaaccggatgaattctttggcatcttcgacaccttcctgcaggccttcttggaggcccggcaggacctggaggccatgcggaggaggaaggaggaagatgagcggagagcacgtatggaattcatgttgaaggaacagcgtgagaaagagagatggcagaggcagaggaaggtcttggctggcggcgccctggaggagagcggcgagtttgatgacctagtgtcagccctgcgctctggagaagttttcgataaggacttaagcaagttcaagcgcaaccggaagcggcccgggagccaggttccggaggtcacccgcgagcgggccataaacaggctaaattattgacttggagactggggactaaccacagaccagcaagcctggagacagggattagaagaaaggagttgaatagtaaaagcaatgacttaagtgattttggaaaaaaaaaaaaaaaaaa

SEQ ID NO: 75 - DNA, 3510 bp, Homo sapiens
cgtgggtcggtccctccgacacacgagctccaagccgctcctctcttttcctgaagcccttcaaggcggtcttcggtttccttctttccagccgatcctcttcagccctgtgaagatggtggaccgcgagcaactggtgcagaaagcccggctggccgagcaggcggagcgctacgacgatatggccgcggccatgaagaacgtgaccgagctgaacgaaccactgtccaatgaggaacggaacctcctgtcggtggcctacaagaacgtggtgggggctcgccgctcctcctggagggtcatcagcagcatcgagcagaagacgtctgcggacggcaacgagaagaagatcgagatggtccgagcctaccgggagaagatcgagaaggagctggaggccgtgtgccaggacgtgctgagcctgctggacaactacctgatcaagaactgcagcgagacccagtacgagagcaaggtgttctacctgaagatgaaaggggactattaccgttacctggcagaagtggccaccggggagaagagggcgaccgtggtggagtcgtctgagaaggcctacagcgaagcccacgagatcagcaaggagcacatgcagcccacccaccccatccggctgggcctggcgctcaactactcggttttctactatgagatccagaacgccccggagcaagcgtgccacctggccaagaccgccttcgacgacgccatcgccgagctcgacactctgaacgaggactcctacaaggactccactctgatcatgcagctgctccgagacaacctcacgctctggacgagcgaccagcaagacgacgacggcggtgaaggcaacaactaaggccccaggtggactggcagcgcccgctgatgctactactgcagtctttatttttcccatgagttgggggtcgggtgggggaggggaaagggaggggtgaccttcccagggagaaacccacgactgtcctgtctttgattgcctctttgacatttttgccaaaataccactagtggaaagtcaggctagctatgctggtatgggagtagcagcctcacacagacgtccggactgttgtgcaggctcggaacgagcaagcggagctgtctgcctttagtttaacttattgctagacggtagggttctaagaagaaaaaccggagatggatggccctcattcagtgcgttctgtggttccagtaaggattcttctgtccatgcccttgtcctcagttccgtggctgttttctctctttggctgtgctttctctctctctctctctctctctctctctctctctctctctctctctctctctctcttttttccccctggggtgtacaccagagaagttatagtttaaattacccaactcgacaggcttagagtggaaggcacaccttttgttagtgagttagacgtggccctgtatttggaagtctctgacgggttgacagtattaacactcaattgtagtttacagtatttatacatgacagccatacatgacgccaagccatgggttctcagttttcctttgtcaggtcggctttcatccgcgtccgggttggtctggctcttcatcatttcgtaacgtggggagagtcagagctccctgttccgttttggttgatgggtgtttgggagggggctgcggggatacttttttttttagttcttctttacccttaaggtcagcccatcacaaaaacagaaaccgttaaataccccccacacagagaaaacggagaaaattcaggtctgtatgtcatttatcagagactctgctctcagcagccagggctccgtctcagtgtcactgcctgttgaatgttcctcagcacccaagtctactctgcaccttgtgctagcagcatgcaggtgccccgaggcgtaggactggagtgtagcgggtgggggattatcatccggtgccagcagctgagcctacagggaacccactaggaagaggcagttcacttgaggcagcctggtccctaagccagttgtccctgcctccctccagccccggcactcctttcccaagtgcttagtggagggtagccgaggttcggcaggaggccatgctcaccctgtttgacctaagctttcctggtttcccctttttgaatgaaatcgtagttgttgagattggcttctctctcatgaggtcgctgcacatgtgacattgaaggaagctctgctgctatgtcctacatggtccgtctcagccctaggtccctcattctcactgtggatagccctttccataaacacgcccggttgcatgcagagtgtgcagtttatgtcttctggagaactcatggtctcgtgggttcccaagacgggcagcgccccaaacttgcttaacttgtcttcagaggcccagcagccactttgtcgcccacttgagctggtgccgtggctgcggaaggatgacgcccctctgcacagtcccagcatgccctttctcggagcccctccccatgctagtgtgccagcaccccctaaccgagaccgcgcaaggccgcagcagaggaagcttggccaccagagtggacagaggggccggagaggcttcgcccgggggtttgtctccttgtccgcagtgagcgtgtctgtgtcccgcttgtactgtttggaaatgacctactggaattgcaaaacctagtttctctttacatttcagccttgacctttgctgtgttctagaacagtcatgtgagggtagaactgaagagggagagcgggagacctgccgtgagcttggttccttctccttcctttactgttgagaagtgtgcctgcctctgatgctgtccccagtgccgcgcggctgctcctccatcctattgatacagtattgtgcatgctggtgttgtatttctatacggtagcttgacatttattaccttacactgtaggtgttatgtatcctatgacaaaaaaacttaagacttcaatttttttttttaaacttcttttttttaatttaattttttcctttcaggggtaaagtttgctttaccaaatagtgattgtaacagattgatctgttttggatgttgctatagtgacatgcagttctatattttgtttttcaggggggaggggacaaaagaaacaccagtgttagcttactcttaataatgtctggtgtttgtcatggtgaaactataactatcgcagtgcaggagaacgcgcggacgttctcagaacgcgcctctgtgctttcttgtcatgttatgcagtgaaaatatttttaaggtctaatcaagtgattttttcccccccaactccgtgtttcgctaaggaattatttcgcacacggaccattcttagcagtttcctcagtgatggaatattcatgaatgtgagtcattatgtagccgtcgtacattgagcaaaataaacttacagatctgaaaaaaaaaaaaaaaaaaaaaaaaaaaaaaaaaaaaaaaaaaaaaaaaaaaaaaaaaaaaaaaaa

SEQ ID NO: 76 - DNA, 3874 bp, Homo sapiens
gcgggaactttccccggggtcggcggaggctcggccgtgccatggaggagggggctagcgccgcgcccccgctcttgaactgggactacctggagcgctgcttcgcccgccgccgcgtctgcatctccttcggcctgtggatctgcgccgcctgctgctggatcgctgcccacacgctgattctctacctgagatgcacaaagaagtgtagccaagaccagtcgacactgtgcgctgcgttctgcctcctaacgagtctgtgtgacaccgtgggggcgattctggccaggcagctcacgatccaggtgttcactggggcctacctagcagctgtcgatttcatgaacttcatgttcactctctttcctgtctgtggatccaaatccaagtccaagtccggtcagagcagcagggagaggagaagaaggcatctgagagccagtgtgttcgccctggctctgcctctgagcctgggtccaggctgggcaatctggaccgctatccccaaggctccagccccagtccgtgggccacaacggaggctcctgggaagccttctgcaggaaaacccggaagtcttcggctacctgctgggtgctattgctgcctttggctcctgggcatcccggatccctccattctccaatatcgtgagactgggttggggctggggctatagggcatctaggggcatcaggagatcctggaggtgggatgtccctgtgtggggcacacatgtgttttttctgaggagccagtatatgacagcagtgaggggcgtatgtgcatgggtctgtgtgtgtatgtgtgagtgagtctgtgtgtgtgtgtgtgtgtgtgtgtgtgtgtgtgtgtgtgtttatgaaggtccatggctaaacagtacagtcctgagacccacaggaacattccaggaaggctgggtgtttctccttacggaaaaaggtcacaggggtggaacaaactagaggctcacaagagaacaagaaagacacaggaagtctaacttggttagctacccaaggccttatcggcagatatttccagatctctgggctgagggaagacttacgagccaggcctgttgttgccaaatgggacctgtccctgggagctctgagtcccacggaagacacagatcacggtgtggtgggagggaggtttccttgctctccgaatagaggaagcaagtgtttagggtgggaagcaggagagaaggctcctgtgctgcgatggagtgagggcagaaaccattccagtgtcaggctgcaggtagtctttcacagtggtgcagagggagagtgggaccagaggaggcaacagctaagaaagggatgtctgtacccaggggaggacgtggagaagccaccatacaggacagtgcctgaatgagctcagggattggtgtagacactgcccagccaggttttctccatagaagactctgtggtaatgcccctgcctcggtaataggaccagcaccacgcctgatactagaaagtgagctgggcctgttatttaacccgaggagaagcactttcccaggagcaccgtgctttagtgccagggtcagaagaacaagggctgggaggagggcagcttgcgactgagcatgaagagccgggctgtgctttgaggcctgagcctgcaagtcctgtctccaggctaaaggaagagacagagatgctgccctggctgcagcagtctctgggtgagcttcacatgtcagggagaccttccatggatccagggcagtgtcctgacctcctgttccagttccctcgggcctgcagcgggatggtcaggctggggaactcacaggccctcaatagacctgagaggcttccactgtgctgtgggaaccatgcagaggggcaccattacccccagccattactggggccagggaccagacccaccctcacatccaggcactccacagttaggtgcttgactgggtctctctattcaggggccttcagcaggtggaggccagaacagtggctgggaagaaactagctgagatacaggggagatgcagcagtccctacagtgccttgtttgtttgttctgtgctgaacattgagccccagacctcacatgtaccaggcaggcagtcttccacggagctgcatccacagtccatctgagtattccttggggattcaaaacaaaaatgaaagaaaatcagccaaaatatacttttctcccgaaatttgttccattccttagccaacaccctaaatagttctaaatgccgccacttagcaagattttaaaaccagattatacatgttttatacttgcatatatatatatgtaaatttaaatatatattatgcaatttagtacatagtctttatatgcacatataaatgtatatataaaatgacttaaatattaatttatatttattaatttattaattgctggagacatggcacatgtataccatggctcaggttttacgaccacaggacaactttcagacctttctttcttctttgtgggttccaggcatccatcaaccttaagttgtcaacagcaggtgtctttatctgctgagccatctcactggccctaaagtgacctcatgcctacagacaactactaccatgaatattgcatatgtccctttttgtgagccagtccatcctctcagaatgtctccgtggcttcacaattccatgtcatctctcccttgatctccctgtttggtgtggttccacaggggaatagctggccagtggctctttgtaacccattctccctcaccttcccccgctggcatttccaagttcctacccactgtcagaagggacggtctcaggtctgctcccaggagtgccccgaatgtgtagagtctcacgcatccctccgtgtcaggcaggtgccaggtgttagggtccagaggagaatcagtattccataggctgcaaacagtaagaaaagcccccaccccgttttaagatttaaatcaggttctgaggagtttgccaggcaaaagtagcagcttgaagaacagctcgatcgatgcctagcagaaggagcgccctttgtaagtggccgctcatagagggcttgcccaatgccaccaagagatccagtgtcctcctgcccggcccctcctctctatgcagtgtcggggcaagtcactctcctatatccacctgtggacccggttcctgtcagctctggctggtctcctctacgcctcggccatcgtggctcatgaccggcagcctgaatacctccttcaggccacaccctggttcctgatttcactgggccgagctgcgctggaccttgctatcatctttctgtcctgcgtgataaagagcaggatgagccgagccttcagatttgctacagctgaagccagagagagtgccgacacacaagcccttttgacctgtgcagagaaggaagaagaaaaccaggaggccaggacggaggacaagaactcagactgggtgcctctgaccagcctgtcacactgcaagcccctgaggacaatgacagccatcagtcgctacatggagctgaccattgagcctgcccagcaggcaggctgtagcgccaccaggctgcccggtgacgggcagacaagcaccagagacgccgcctcgcaagagcctccctcataccctcccatccaggtcatccaggccagagtgtcttccagcagctcctccgaggtctcctccatcaactcggacctggagcagaagtattgggaagccctaaactcggagcaggcagctggccgtgtcctcacttgcaataccagatgggcatatttcccgacagtcacctgtgcctgtcttctatcacccaggagtgtttgtgacacaagaatacatcactcacccac

SEQ ID NO: 77 - DNA, 1639 bp, Homo sapiens
gagcaagaaggtgcaggatgtaggcctgcagaattgtcagacccagaggcggcttttgccactggaagaatttccaacataaaacagatgatcagtttgggacaatcgattctgcgaccagagtttataaagctgtccaagatctgcccagtcccagatgtcctgggtcctcagggcggctgtggtctgcgcccttctcctgcagctggatgccagaccatcctggactcggatccctttgggttcacactcgcttaggtatttctacaccgctgtgtcccggcctggccttggggagccctggttcataatcgtcggctatgtggacgacatgcaggtcctgctcttcagcagcaaggaggagactccgaggatggcaccctggctggagcaggaggaagcaggtgactgggagcagcagactcgtatagtcacaattcaaggacagctgtctgaaaggaatctgatgaccctggttcatttttacaacaagagcgtggacgactctcacacactacagtggctgcaaggctgcgatgtggagccagatcggcacctgtgtctctggtacaaccagctcgcctatgatagcgaggatctccacaccctgaacgaaaacccaagttcctgtacagtgggaaacagcactgtacctcacatctctcaggacctgaagagccactgctcagatctgctgcagaaatacctggaaaaagggaaggagaggctgctgcgttcagaacccccaaaggcacatgtgacccatcaccccagacctgcaggtgatgtcaccctgaggtgctgggccctgggcttctatcctgctgacctcaccctgacctggcagaaggatggggaggagctgacccaggacatggagtttgtagagaccaggcctgcaggggatggaaccttccagaagtgggcagctgtggtggtgcctcttgggaaagagcagagttacacatgccatgtgtaccatgaggggctgcctgagcccctcaccctgagatgggagcctgcatggtaccaaaagccttggatttggattgttgccatggttttcattttgctcatcatttgtctctgtgtggtttgcatatgcatgaagaagaatgcaggtgggagaggaaggcgtgacacccaagaagcaggcagagacagtccccaagactctagcaagactgttgtggatgatgaggagatgggggtttgcttttggaagattaagtactgtaagacttgtctaggccactccccaggaacttcagttggcgagtctttactgtcaccttgactggatttaggatcatctgggagatgcccctttgaatggctgggctgtgaggacagcagaccagttcttgccaccctggacagaaacacatctcacctttctggctcgaggatctgaacacctgtctcttgcctacttggcttctagtcaggcattttgtcaccttgtcaagggtcccagggacacaaaactccctcctctcacccacagcactctgggtcctaccctcagtgcttcagggacatttaatcacgtcaaattgggatcaatggctttgatgcagaaaagaactgtggactaatagagatagggtttaattaaaaaatatatctttttaatttt

SEQ ID NO: 78 - DNA, 2557 bp, Homo sapiens
tgctttctaggatgggggtgtgagtggaaaccagcgtcgctatggcaaccaggccgggtggtcgggccctggagccctgggagccagcgaaggcgagaggcggggcagcgggaggcgccagggctgcgcggcggggcgggggcctgggtctcccgggaggagctggggaacaccgggcgcctctcgcggaggtgcacgccaagttctcgggtgagtgtgcacgcgtgtgagctggcccaaggctcacgggcgggcgtgagcacgagcacacgtgtcgagtgtgcactcgcgtgcgttctggggcccgtgtctggccctctgcgccccgcgcccctcccggccgcccgcagcagctcccgccactcccgccccgcgccgcttctcgctcggtcctcgcccgccgcgccgcagtcgccgccgccgcccagcctgcgcccacgggccgcccgtgccgggcacctgttggaggctcagggacaggactgatctggcaacccacccagccctttgtgaagccaggcctcctgcctgcccaccagccagcgcagatcatcttttccctcgacacccaggaagggagggcagtgaggttcctacagaccccagccggccactccagctctacacccgtctcccagtcagaggtgctgcatgaggggccgcaggggcgatcgcatgaccatcaacatccaggagcacatggccatcaacgtgtgccctggacccatcaggcccatccgccagatctccgactacttccctcgcagggggccaggaccagagggtggcggcggcggcggtggcacgggctgcggggaagccccagctcatctggcccctctggctctggccccccctgcggctctccttggggccactacacccgacgatggagctgaggtagacagctacgactcggatgataccaccgccctgggcacactggaatttgaccttctctatgatcaggcttcctgcatgctgcactgtagaatcctcagggccaagggcctcaagcccatggatttcaatggcctggctgacccctatgtaaagcttcacctcctgccaggagcctgcaaggccaataagctaaaaaccaagacacagaggaacacactgaaccctgtgtggaatgaggagctgacgtacagcgggatcacggatgatgacatcacccacaaggtgctcaggatctctgtctgtgatgaggacaagctgagccacaatgaattcattggggagatccgagtgcccctccgccgcctcaagccttcacagaagaagcattttaacatctgccttgagcgccaggtcccgcttccttcaccctcttcaatgtctgcggcgctgaggggcatatcctgttacctgaaggagctggagcaggcagagcagggacctgggctgctggaagagcgcgggcgcatcctgctgagcctcagctacagctctcggcggcatgggctgctggtgggcattgttcgctgtgcgcacctggctgcaatggatgttaacggctactctgacccttatgtgaagacgtacttgagaccagatgtggataagaaatccaagcacaaaacatgtgtaaagaagaagacactaaatccggaatttaatgaggaattcttctatgagattgaactctccactctggccactaagaccctggaggtcacagtctgggactacgacattggcaaatccaatgacttcataggtggtgtgtctctggggccaggagcccggggagaggcccagaaacactggaatgactgtctacatcagccggacacagccctggagcgctggcatactctgaccagcgagctgccccctgcagcaggggcttaccctttggcttgagtgaacagcagctgtccatcacaggccctctggggccgggtccagtacccaaccttcgcacgagtgtgttgcacgtttacatggggtgggatgcccctgccccacactacctgtcttatttttgtgagtctctgtgacgggcctgtcttcttgtgagggactgtggagagctatattcacatatgcaaacttcctcctgcctgccttgctgttacctgcaaatatgcaaccacccgtgcacagggccacgtgggagtctcctgccagtgccccaccccatcctgcagctcctttcttggcctttccaggctgcctggtcccctgccccacagggagggggtcggattagggaagattagaggaggacgtttccaaatagaggaaaggacaaggaaagaaagagccaactctttaatacagagccttcactaaatcccagccctgcgtagctgctcttctaaaggggcggccaccgtaggtctctacctcccgaagatgtagcagacccttttggccactcgtttaaataactgttccctggatggggctgggatgtaagggcaggaccctgccaccttcctcggggacattgtgcatgtttacgccttttctgattgtgtcagtggccgaagcatggcaactgggcatcaataaacatctcttgaggaaaaaaaaaaaaaaaaaaaaa

SEQ ID NO: 79 - DNA, 2966 bp, Homo sapiens
cggacgcgtgggtctcttcggacccttttcgcctgtgggtgacccgacctcaggggcgggtggcaggtgaggagcgggtcgcagtgaggagggtcgccggtaccctcgaccttcccacagagggcggcgtcctcgcgtaatcaaccgctctcggccacgcagagagtagccccacgaggggctggctccttcgggctttagccaattctgtgtgctccctgtggttttggacaagacaacatggaaacagctaaagatggtgagcagagcccaagtgaggcatctccttcagcccaggcaggtcctgaaaacatcccggagcctatgtctcgggaggaggagagccagccattgtaccatgaagagaccattgatcttggtggagatgagtttgcatctgaagagaacgagcccacatcggaagggtctcacaacttcggagataagcttaatgagcacatgatggaaagtgtgctcatctccgactctcctaacaacagcgagggggacgttggcgacctcggctgtctacaggatgttggggagccccccagaggtgccactgatcaccgactgcccagtagcacagacaaagaagtggtggacactctgagtaatggcagtgaaaccgatggagacgacactcccagagacatctcagacatgacacctgatagcagagcctctctgaaggaagactcaacacaggaagatgtgacgagcatgcctgcactggagaatgctgccacggaggaagtcgggcctaaggacagcctcgcaccccgagaagagcagacctcagaagtgtcctccaatcagtcctcctccaaagacgagcccttgcccgtctgcaccatcttcagccaggctaccgccactccctcccagccacacctgtttctacaggatggcttcgagtcccagatggtgaaatcacccagtttcagtagcaccagtgagatgtcagccaagacccctcctccgatggtccagcccagtcccagcctcagtacattttttggagatacaatgagctctaattccttggcctctgacttctttgattccttcacaacttctactttcatttcagtcagtaatcccaatgcaggttccccagttccagaaaagctgtcttcactcactgcccctgtaggagagaagtccccggattccaccagcccatcttactccacaaggatggatagatcggaatcaggtgtttctcgggcccctctggatgtccctgaatctccaaagccattttcacagatccaggctgtgtttgctggaagtgatgaccccttcgccacagccctgagcatgagcgagatggaccggaggaatgacgcatggcttcccagtcaggcgaccagagaagtcctgatgtcagtagcaacccagcagtatggcactgtgttcatagacaaggagaacctcactatgcctggcctcaagtttgacaacattcagggagatgcagttaaagacttgatgcttcgcttcctgggtgagaaggctgcagcaaagagacaggtcttaaatgccagctcagtggagcagtcctttgtggggctgaagcagctcattagctgcaggaactggagggcagcagtcgacctgtgtggacgcctgctcacagcccacggacaaggctatggcaagaatgggctgcccaccagccacaccacagactccttgcagctctggtttgtgaggttggcgctgctggtgaagttgggccttttccagaatgctgagatggagtttgaaccctttggaaatctggatcaaccagacctatactatgaatactacccacacgtgtacccaggacgcaggggctccatggtccccttttcgatgcgtattttgcatgcagaactgcagcagtatctggggaacccacaggagtccctggaccgattacacagggtcaagaccgtctgcagcaagatcttggccaacttggagcaaggcttagctgaagatggtggtctgagcagcgtcacccaggaaagcaggcaagcctctgttcagctgtggaggtctcgtctgggccgtgtgctgtactccatggcaaactgtctgctcctgatgaaggactatgtgctggctgtggacgcctacctcacagtaatcaagtactacccggagcaggagccacagctgctgagtggaatcggccgcattctcctccagatcggagacataaaaacagctgaaaaatattttcaagacgttgagaaagtaactcagaagttggatggactgcagggtaaaataatggtcctaatgaacagagcctttctgtaccttggccagaataacttcgcagaagctcacaagttcttcacagagattttgagaatggacccaacaaatgcagtggccaacaacaatgctgctgtgtgtctgctttacctgggaaagctcaaggactccctgcggcagctggaggccatggtgcagcaggaccccaggcactacctgcacgagagcgtgctcttcaacctgaccaccatgtacgagctggagtcctcaaggagcatgcaaaagaagcagagcctacttgaggccgttgctagcaaagaaggggacagcttcaacacacagtgcctgaagctggcttagttccctccaggtctctgtacctgcagacacttgagcagatgggtcagtaaaagcatttgatgggtgtgcaggagcatgccttcggctggctgggctaccctcagacccgctgacctgcctaccatgtagttgcacaaaactgcctgttggtgaaggtgagccatatgcatggcattcagaaacagggaacataaggacgtggggttcccttggtttgttcatggaagctaacttgttctttggtcacttctgaatactcgtttggtaaataatactgcctgttctcactataggcctaaaaaaaaaaaaaaaaaaaaaaaaaaaaaaaaaaa

SEQ ID NO: 80 - DNA, 5242 bp, Homo sapiens
ccggcagtcagaacccgaggtggaaaggcaggctgccgactgcctgtcggaagccgcagggagtgagaaagggtgtcgcagagagcgcggcggtggctgagatttcttctgctgaagagaggaaatctcatccaaggccacaatgtcacttcccaattatccgagtgctgcctgctcaagctaacactgccagagtcctgctccctcgggaggagtcaagcttacttggcatgaggttcctgctttcctggggttgagagaagcacaactatggcatctcttgatgacccaggggaggtgagggaaggcttcctgtgccctttgtgcctgaaggaccttcagtctttctatcaacttcagtcacattatgaggaagaacacttggaagatcgtgatgtcaaagggcaaattaaaaatcttgttcagaaggctaggaaagcaaagaacaagttgttgaaacgagaaggagatgatcgagtggaaccagggacccaaggatatgaatcattcagttatggtggggttgatccttacatgtgggaaccccaagagcttggtgctatgagaagccatctttctgacttcaaaaaacatcgtgctgcaagaattgaccactatgttgttgaagtcaataaattaatcatcaggttggaaaagcttactgcatttgacagaacaaatactgagacttcaaagattagagcaatagaaaagtccgtggtgccttgggtcaatgaccaggatgtccccttctgtccagactgtgggaataaattcagcatccggaaccgccgtcaccactgccgcctctgtgggtctatcatgtgcaagaagtgtatggagctcatcggcctgcccttagcacataagctcaccagtgccagcaaggactctctgagcacccacaccagccccagccagtctcctaacagtgtccatggctcccgccggggcagcatcagcagcatgagcagtgtcagctcagtcctagacgagaaggatgatgaccgcatccgctgctgcacacactgcaaggacaagctgctcaagcgagagcagcagatggacgagaaggagcacacgcctgacatcgtgaagctctacgagaaacttcggctttgcatggagaaagttgaccagaaagctcccgaatacatcaggatggcagcatctttaaatgctggggagacgacctacaatctggagcatgccaatgaccttcgagtggaagtgcagaaagtatatgagctaatagacgctttaagtaagaagatcttaaccttgggcttgaaccaagacccttcaccacatccaaacactttgcggctacagagaatgatcagatactcagctacattatttgtgcaggaaaagttactgggtttgatgtcactgccaaccaaagaacagtttgaagaactgaaaaagaaaaggaagcaggacctggagcagaagaggaccgtggagagacaggctgctctggagtcacggcggaagcttgaggaaaggcagagtggtttggcatctcatacagccaatggggacgtgaggtctcttcgaggcatccctccccccttgcgaaaggctgagggctggctcccactgtcagaaggtcagggacagagtgaagatcctgaccctctcctccagcagatctataacattacatcgttcatcaggcaggccaaggctgcaggccgcacagatgaggtgcgcacacttcaggagaacctgcggcagctgcaggatgagtatgaccagcagcagactgagaaggccattgagctgtcccggaagcaggccgaggaggaggagctgcagcgggagcagctacagatgctccgcaagcgagagcttgaacgagaacaggagcagttcctggcagcatccctgcaaacacggactcgtgttctagaactccgagaagttatacccttccaattggaggccagtcgagggcctcacattgatctctcctattccttggatcaggactcctccccagttcagagcagcacagctcctgatatccttacacctggctcagctctagcacccatgcacttgtggtctgggcccccagcccttggccaggaaacactccctcagagcaccatgtcacagcaaagtgataaggcctcactcaacccgtttgatgaagacgacctctccagccctacagagggtgctatcagccctgctgctgtagaggctttcctgggccctccagctgctgtcaccaaagaatacaatccttttgaggaagatgctgaggaagaggaggtggcagagttgggggcaggaaatcccttcaccgacccagacagtccagcacccaacccttttgatgaggatgacggtcccaggcctgccagtccagccgcccctggaaacccttttgaagagtgcccctccaccaaccccttcgaggtggacagtgatagtggcatggaggccgaggaacacatcgaggaggagctgctcctgcagcaaattgacaacatcaaggcttacatctttgatgccaagcagtgtgggcgaatggacgaggttgaggttctaacggagaacctccgggagctgaagtgcaccctggctaagcagaagggggcccctaattgacagcaatgggcaggaactgggcagagtcttgggcagcaggatatcaagtgtgaggtggggatgggcaggcatctgaacacacacaggttggggtgggaacagtagtctcttgctgtgcactttgagacatttccactacagttgagcaagaacgggaaaaacaatcggcattcatgtgctggtttttctgtattctttttttaatgaagtattccattttcaggggactttaccattttattttcaagatacagctctgttgggccaaggcagaaatggctcatgggcacctcttagttcattttctgcggactgttgttggtcattatgcagtattatagggcagcccttgacaggcccttcctgtagccaggataggcagggctctagcagaagtctgtcctgacttggttgttccctttcagcacgtcacttagggagatactgctgtgaagcccagtgggaccagggcctctttatggatggcaggaatgttgccagtgttgtcccctcccagttaaaggaagtaaaattcccaagtgtgagggtagtgggctggtggtgtcagtgtgagcttgactgtgagatagtagtgttcaactttttggcggatgttatggtttggagaagtttctacttatgcagctgtctgatcccctgggaacctccttccaggtctttggtagaatgaccgttctgcaggtcacattcaagggcctgttgatttggaattgtgactgggaagcttctgtggagtattagcaggtgatggaacacttctgagtcaggatgattctagtaacttcaaggccctccagtcagccctcttccttgtggtttatcctgacctgggaaactggtaagatgtactgagaaatggaaatatttgaggcacagcctgagaccattccacagccctgaagtacttgaactcttgatagaaaacagaaagggaagttatgtagcttagtactttggtttacaggaacagatcgattgacagatgaccttgtaaggttcttggtgtgctcagctctgggtaatttctttggtgatgatactctttagttaatggacaataactgtccactagtcactttcccctcagctttctctgggccatgccttttgttggttactggtgtataagctgaaagtcttctgtcacattctggtgaagatccatctaggtggtaagaatgttatctgggtgcaaagatctctatgttggcagctttaatcccagttcattagtacatgttggggctttggctggatagagagccctgtatggggctatgggaagggctggagaaggagggagcgtatgaaccctttgagttctgctgacaggtgtttactggcctgctagtagattaccagccatttgccagctctgttggtgtcaagatgttctggaaggtaggcagcctggcattttatgttacctggggcaggctacctctttctaaagcccaggctccaggcatgcccaccatatctgccctgcttggtctgctattgtgtcatggacaaagaggcagtgctaacacaagaatgaagctaaattctggtttccaccattacttttccagactcaaatcaggtgttcagggtggtacaactgtagacaatgcttttcttataagactgggcttcttaattagctgtgagtgactcgatgggcagtagtggcacatgacttttatcccagcacttgggaagtagaggcaggaatttctgagttccaatccagcctggtctacagagtgagttccaggacagccagggctacacagagaaaccctgtctcaaaaaacaaacaaataaataaataaacaaaaataaaaattgagaaaaaaattatctagtgacttattaaatgtacttctttctggaactcaccccaggtagcagtctgactgagagcatggagaaaacaaatgcttttagactgactgactattaaaccgggtgaacccagatcccagagggtttcctgagtttctcattgaattcctcgctgaccatattctgaaagtccaacagaacacgagagacttgtctgccccttgtctggatcagctcccagaatgcatgcaggtcagagttggtttgcagagggggtttggcctgtgagggaaccacagtgccagcttcttaccgtgggtgaatactcgatgattcccaaggatggagcacgtccttgatacggtgttggcagatcccttaggtgtaggtcctgttcatagtggacaggcttcttgcttattcccttctaacaacactaaggaactggagcctcatcactgcacttaccacatatcatgtctttgtcgtcagacagtaaaacatctttctaagtcatcatgagccatattgtgtagctatgccatgcggttcaaattgtggaattggcctcggtacgaggtcagactagaggagctcggtgtgctggtaatcatgatgactgtcagcaagatggtcattgatgcgcatgcactacactggggccttgtattagaggcttttattaaaaacattaaaatatcatttatgtaaggcttttattaaaaaaataacatgttcatggctggaaaaaaaaaaaaaaaaaaaaaaa

SEQ ID NO: 81 - DNA, 430 bp, Homo sapiens
tgagagcacgcatgacatgctggggcaagcgtgatttttacagcacgcttcggaagctcgatcaagaccgctttgctggatgtttccagaacattcttaaggaatatttgaaaaccaggattggaaaaaaacccaaaacaacaacaacaacaacagattagatcaactctttgctgtggagattctggcttccaaaaatcctgctgactgcagcacagctgatgatgtcattgcatcatcacagcggaactggaattcctactgaaattacacttcacgacaaacaagacacaagccaagaacgccactcactgtgttttcccacaaataagtgctataatgtcctgtgagaattttgtctgtgcttttagagacatgggaatcatttgacagtatttaattttccagttgtgctccttatatacaaatg

SEQ ID NO: 82 - DNA, 3275 bp, Homo sapiens
agactgcggcgctgctccattcggccgccgggccccgcggagcccccatcggtgaggcgaggctccagcaggagacgagcgccccgctacactgcaggggtggcgtagcagcgggcggccgagcgcctgtgagagccaactgcgggaggccagcgtgggagcagagatagaaccaggagtcggacccgcgtcccctccgcggaaccccggttcctgtcactgtgggggacatcatggccgtggacgtgacagaatatcatctgagcgtcatcaagagccccccaggctgggaggtgggtgtctatgctgccggggccctggctctgctgggaattgctgctgtgagtctatggaagctctggacatcggggagcttccccagcccctccccgttccctaattacgactacaggtacctgcagcagaaatatggagaggcctatgtggaggctaagctgaagcgggtccctccttggaatgaccagcggaccactactcggggaccacccagccgcaaaggcagccttagtatagaagacacctttgaaagcatcagtgagctgggacccctggagctgatgggccgggaactggacttagccccctatggtactctccgaaagtcacagtcggctgactctctgaattccatctcctctgtgagcaacacctttgggcaggatttcaccctgggccaggtggaggtgagcatggactatgatggagcctcccacaccctccacgtggcagtactacaaggcaaagacctcctggagcgcgaggaggccaccttcgagtcctgcttcatgcgtgtcagtctgctgccagacgaacagatcgtgggcatctcccggatccagaggaatgcctattccatcttcttcgatgagaagttctctgtccccctggatcccacagccctggaggagaagagccttagattttctgtgtttggtatcgacgaggatgagcggaatgtgagcaccggcgtggtggagctgaagctgtccgttctggatctcccgctgcagcccttcagtggctggctatacttgcaggaccagaataaggctgctgacgctgtgggagagatcctgctgtccctcagttacctgcccacagctgagcggctgactgtggttgtggtgaaagccaagaatctcatctggaccaatgagaagagcacagcagaccccttcgtcaaagtatacctgctgcaggatgggaggaaaatgagcaagaagaagacggctgtgaagagggatgaccccaacccagtgttcaacgaagccatgatcttctcagtgccagccattgtgctacaggacctgtctctccgggtgacagtggctgagagcagcagcgacggccgtggggacaacgtgggccatgtcatcattgggccaggggtcagtggcatgggcaccacacattggaaccagatgctggccacactacgcaggcctgtgtccatgtggcatccagtccggcgaaactaacagccagagcaggccaggttgggcatcagagcctcaggagcctggcaccatctcagttctatccagcgatgccctctccatagccagctggagctgtgaacaccagggctgccccacctcctgggggatttctcatcagccatcttggatcctctgtccagactgtcatggaagaatgggtgtggctcttcatccggggacccagggtaatttccccactgaggtctacctgtagctggctggggacccaccagactgacctactgtattgaagagccttaggaagtatgtcccttggcctgcagaaggatgtgggcttcagaacttgccaactccccttctgccattctcctagaggacagctgttaataaatcttgaggggatagttcctgtggtggcaaggctaatattaacaagacatagcactcaggatgccaggaacccaagatgccctctctcatgctggcaccgagtaacaacccgggcagatgaacttgggaaggagccctgaggggtggggtaacagcgaggctctctcagacagggaaggagtttcactttgagggtgagagaaaaaatagcatcccatctttaccatgggggtcgtggggatcatggtgagcagtcacacaatagaagagacacaaactaccccatcccaaacacagactctgcccactacagtggacacaagaagaggccaggagaccaggcccaacagaagccagtaggccgtatgaaggctcccctgaccatccactctgagggaaccagaagtccccagtggcccaggtccagatccttgtgtggatccaaagcaaatcagcacagattccacagacaccagtgttggctcgcgtccctccaggccaggcttctctagaaaggaaggtgtcaagccggtctaatccaaccctaggaattgtaggtcaagttccctaaaactaagactgctccttagtcttgagccagtaggaactgggggaaatggtgttcctaagcaaagggcttggttgtacaccactaccagaatcgttctttggtcagagttattgaggggatcgcatcatacgtgaggtgctggctgtcacttgagaaaggcagttctcctagagaaatggtggcttttggctccttctctctggaacctgaagatttttttgttttttgatttttgggttttttttttttttttccgagacagggtttctctgtgtagccctggctgtcctggaactcactttgtagaccaggctggcctcaaactcagaaatctgcctgcctctgcctcccaagtgctggaattaaaggtgtgtaccaccacggcagactcgaacctgaagatttaaaagggcagtgggcgtgggagtggtcagaagagggctttggtccttggccctagtgaatcatggaggcccagatggaagcaggtctaataatggtcaatgataaggagaagggcagatagaatagggacacaggtcaacaggacaccagccacagctggacccactgaagcttctcaccctgtggccaatattcaagaggtgaaaacgggccagggatagtggtggctgggccaggccttgtcatctggaagcccccagactctttgtaatccaatggctttgcagggaagaccacaccccccaccctccatccccttattccttctttccttccttctatactgcatgaaacagtgtgcccagatggctgtggtggcagccagggatgattcttgaaacaaaataaaactggggcaggaaggaggc

SEQ ID NO: 83 - DNA, 7318 bp, Homo sapiens
aagcactcatagtcagctggggatacaacccctaaaaacagcacacccaccatgtttaacctcatgaagaaagataaggacaaagatggcgggcggaaggagaagaaggaaaaaaaggagaagaaggagaggatgtctgcggcagaactgcggagcctggaagagatgagcatgcgccgtggctttttcaacttgaaccggtcctccaagcgggaatctaagacgcgcctggaaatctccaaccccatccctatcaaggtggccagtggctctgacctgcacctgacggacattgactctgacagcaatcggggtagcatcatcctggactcaggacacctaagcacagccagctccagtgatgacctcaagggtgaagaaggcagcttccggggctctgtgctgcagcgggcagccaagttcggttcactggccaagcagaactctcagatgattgtcaaacgtttttccttctctcaacggagccgggatgagagtgcctcggagacctcaaccccctcagagcactctgcagccccatcacctcaggtagaggtgcggacgcttgagggacagctaatgcagcatcctgggctaggcattcctcgaccaggaccacggtctcgagtccctgagctggtgactaaaagattcccagctgatctacgtcttcccgctctggtgccaccaccacctcctgctctccgggagctggagttacaacgaagacccacaggagactttggcttctcactgcggcgcacaaccatgctggatcgggcccctgagggtcaggcttatcgacgggtggttcactttgctgagccaggggcaggcaccaaggacctggccttggggctggtaccaggagatcgactggtggagattaatggacagaatgtggagaataaatccagggatgaaattgtggagatgatccggcagtctggggacagtgtgcggctcaaggtacagcccatcccagagcttagcgagctgagccggagctggcttcggactggcgagggacaccgcagggagccggctgatgccaagacggaagagcagattgccgctgaagaggcctggtatgagacggagaaggtgtggctggtccatagagatggattctccttagccagccagctcaagtctgaggagctcagcctacccgaggggaaggcgcgtgtgaagctggaccatgacggagccatcctggatgtggatgaagatgatatagagaaggctaatgctccttcatgtgaccgtctagaagatctggcctcactggtatacctcaacgagtctagcgtcctgcatacactgcgccagcgctatggggctagcctgctacacacctatgctggccccagcctgcttgtccttagcacccgaggggctcctgctgtgtattcagagaaggtgatgcacatgttcaaggggtgtcggcgggaggacatggcaccccacatctacgctgtagcccagactgcatatagggcgatgttgatgagtcgtcaggaccagtccattgtccttctgggtagtagtggcagtggcaagactaccagctttcagcatctggtgcagtatctggctaccatcgcgggtaccagtgggaccaaggtgttctcagtggagaagtggcaggctctgtccaccctcctggaagcctttgggaacagccccaccatcatgaatggcagtgccacccgcttctcccagatcctctccctggactttgaccaagctggccaggtggcttcggcctccatccagacaatgctcctggagaagctgcgtgtggcccggcgcccagccagtgaggctactttcaatgtcttctactacttgctggcctgtggggatgccaccctcaggacagagctccacctgaaccacttggcagagaacaatgtgtttggaattgtgccactgtccaagcctgaggagaagcagaaggctgcacagcagttcagtaagctccaggctgccatgaaggtgctggccatctcccccgaggaacagaagacctgctggctcatcctggcttccatctaccacctgggggctgctggagctaccaaagaagctgctgaagctggacgaaagcagtttgcccgccacgaatgggcccagaaggcagcatacctgctgggctgcagcttggaagagctgtcctcggctatcttcaaacaccagctaaaaggaggcaccctgcaacggtccacctccttccgtcagggccctgaggagagcggcctgggagagggcaccaaactgagtgctctggagtgcttagagggtatggcgtctggcctctacagtgagctctttactctcctcatctccttggtaaacagggctctcaagtccagccagcactcactctgttccatgatgatcgtggacacaccaggcttccagaaccctgaatggggcggctcggcccgtggagcctccttcgaggagctgtgccacaactatgcccaggacaggctgcagaggctgttccacgagcgcaccttcctccaggagttggagagatacaaggaggataacatagaactggcgtttgatgacttggagccagttgcagatgactccgtggctgctgtggaccaggcttcccacctggttcgctcactggcccatgccgatgaggcaagagggctactgtggctcctggaagaggaggcactggtgcctggtgcgacagaggatgccctccttgatcgtcttttctcctattatggcccccaggagggtgacaaaaaaggccagagtcctcttctgcgaagtagcaaacctcggcactttctcctgggccacagccacggtaccaactgggtggagtacaacgtggctggctggctgaattacaccaagcagaacccagccacccagaatgcaccccggcttctgcaggactcccagaaaaagatcatcagtaacctgtttctgggccgggcgggcagtgccacggtgctctcaggctcgattgcaggcctggaaggtggctcccagctggccttgcgccgggcaaccagcatgaggaaaaccttcaccaccggcatggccgctgtcaagaagaagtcgctgtgcatacagattaagctgcaagtagatgccctcattgataccatcaagaggtccaagatgcatttcgtgcactgcttccttcctgtggccgagggctggcctggggagccccggtctgcctcttcccgcagagtcagcagcagcagtgagctggacctgcccccgggagacccctgtgaggctgggctgctacaactggatgtgtccctgctccgagcccagctccggggatcccgcctgcttgatgcgatgcgcatgtaccgccaaggttacccagaccacatggtgttttctgagttccgccggcgctttgatgtcctggctccacacctgaccaagaaacatggtcgtaactacatcgtggtggatgagaagcgggctgtggaagagctgctggagtccctggacctggaaaagagcagctgctgtctgggcctaagccgggtgttcttccgggcaggcacgttggcacggttggaagaacaacgagacgagcaaaccagcaggcacctgaccctgttccaggcagcctgccggggctaccttgcccgccagcacttcaagaagagaaagatccaggacctggccattcgctgtgtgcagaagaacatcaagaagaacaagggggtgaaggactggccctggtggaagctcttcaccaccgtgcggcccctcatccaagttcagctgtcagaggagcagatccgaaacaaagacgaggagatccagcagctgcgcagcaagcttgagaaggtggagaaggagaggaacgagctccgactcagcagtgaccggctagagacccggatctcagaactgacatcggagctgactgatgaacgcaacacgggagagtccgcctcccagctgctggacgcggagacagccgagaggctccggaccgagaaggagatgaaagagctacagacccagtatgacgcactgaagaagcagatggaggtgatggagatggaagtgatggaagcccggctcattcgggcagccgagatcaacggggaggtggatgatgatgatgcagggggagagtggaggctgaagtacgagcgagctgttcgggaggtggacttcaccaagaagcggctccagcaggagctggaagacaagatggaggtggaacagcagagcaggaggcagctggagaggcggcttggggacctgcaagcagatagtgatgagagtcaacgggcactgcagcagctcaagaagaagtgccaaaggctcacagctgagctgcaggacaccaagctgcacctggagggccagcaggtccgaaaccacgagctggagaagaagcagaggaggttcgacagtgagctttcccaggcccacgaggagacgcagcgggagaagctgcagagggagaaactccagcgggagaaggacatgctcctggctgaggctttcagcttgaagcagcaaatggaggaaaaagacttggacattgcggggtttacccagaaggtggtttccttggaggctgagcttcaggacatttcttctcaagagtctaaggatgaggcttctctggccaaggtcaagaagcagctccgggacttggaggccaaggtcaaggaccaggaagaggagctggatgagcaggctgggagtatacagatgctggagcaggccaagctgcgtctggagatggagatggagagaatgagacagacccattccaaggagatggagagtcgggatgaggaggtggaagaggcccggcagtcatgtcagaagaagttaaaacagatggaagtgcagctcgaggaggagtatgaagacaagcagaaggcgctgcgggagaaacgggagctggagagcaagctctccacgctcagtgaccaggtgaaccagcgggactttgaatcagagaagcggctacggaaagacctgaagcgcaccaaagcgctgctggcagatgcccagatcatgctggaccacttaaagaacaatgccccgagcaagagggagattgcccagctgaagaaccagctggaagagtcggaattcacctgtgcagcagctgtaaaggcacggaaagcgatggaggtggagatggaagacctgcacctgcagattgatgacatcgccaaagccaagacagcgctggaggagcaactgagtcgacttcagcgtgagaaaaacgagattcaaaaccggctggaagaggatcaggaggacatgaatgagctgatgaagaagcacaaggcagctgtggcccaggcctcccgggacatggcacagatgaacgatctccaggctcagattgaagagtccaacaaggagaagcaagagctacaggagaagctacaagctctgcagagccaggtggagttcctggagcagtccatggtggacaagtccctcgtcagcaggcaggaagcgaagatcagggagctggagacacgcctggagttcgaaaagacccaagtgaagcgcctggagaacctggccagtcggctcaaagaaaccatggagaagctgactgaggaacgggaccagcgcgccgcagctgagaaccgtgagaaggagcagaacaagaggctccagcgacagcttcgtgacaccaaggaggagatgagcgagcttgccaggaaggaagccgaggctagccgcaagaagcatgaactggagatggacctggagagcctggaagctgctaaccaaagcctgcaagccgacctaaagctggcgttcaaacgcattggggacttgcaagctgccattgaggatgagatggaaagtgacgagaacgaggacctcatcaacagtgagggggactcagatgtggactcagagctggaggaccgggtcgacggggtcaagtcctggttgtcaaagaacaagggaccttctaaggcaccttctgacgatggcagcttgaagagttccagcccaaccagccactggaagccactcgcccctgacccatcggatgatgagcatgatcctgtggacagcatctccagaccccggttctcccacagctatctgagtgacagcgacacagaggccaagctgacagagaccagtgcatagcctgggatggctcacgactcttccaccccacagcctctcccaggatagaggggcaccactgcccccacctgactgccgatctgcatggaaaacaccttggctttctgtcagggggactttccaggctgtgggccgtctgacagctccacgcggcagaggtgggcgaagcagagtctctccaaagagagcttccgctcttgcctctgaacgccatgctcttagctcccgctctgggccactatggaccacgtcaggtggcctggcattccatggagccggtggtggacctccatctcagcctcctgctcagggatggtggataggttacctgctgggccgctcgaagtcgctgggccccatgggaaagagtcaggagggagcagtgatgcctcccctctgctggggtgagggctttctcttctcagtgcctgctgtctttttactgtttaatttaaatacccaaacctccatgcagctgcatccctgagagtgggaggggctgccgctgcagctggggcctgtgggatgccttgggaatctcccctccatcttcacttctccagaggcccttttctcctgcagcctgctgcaggggacagcggttagcagaccggggccctggttctttgaaggcagggtggggaggatggcagctgggtgcttctggcttggaccgagccagcctcgttagagtattaagtgcaatgagcagttatttaagagctgatctcttccacctgcacccacccgcggcttccagctctgcctgcctctgaccgtattatagccggggccgctcattcggcgctgaggacctgtggtgggcacagtggaaggtagatgttttggtctgtttatcagactggagtcctaaccctttgtcatctttcttctgcccataaccctgtccccacacacttaccccagacacagcaacaagtggtctgttacttgttacttctcggtttcttaccataaagctcatctgacttttccactggatctgggggccccaaggcagtacctggtgctggacctgtttgccatcccccttcccttctgccattgctttgtactcattgggctgtgctctccctgtggacctgattgatgtaaggaaataaaaggccccttccctcctggcaaaaaaaaaaaaaaa

SEQ ID NO: 84 - DNA, 4273 bp, Homo sapiens
agtccctggaagcagacgtttcggccacagacccagagaggaggagctgacaatcaggaggcgtgagccgcctggagtctgcagaattcgtggtgtgaatgaactgggggcatcttgggcacagggattgccccccctccttccccgcctcgggccacagttgagtagtggggcattttttttcaccttcttgtgaagaattttttttattatttgttgtaaagtcttttgcacaatcacgcccacatttggggttggaaagccctaattaccgccgtcgctgatggacgttagagagggagcgcctcgccgcggaacagtcgcctgcgcgccctcgtcggacccgcggctcctgcactgtgtccccgctcggccctgcgcttgctgctcgcccgcgcgcgccggcgccctctcggttcctgggcacatttccacgctataccaactcctctgcccgagtccgggcgccagtgctcgcttccgctccgggtcgctgcgcccacccgacgcgcccaggaggactccgcagccctgctttggattgtcccccaaggcttaaccccgacgcttcgcttgaattcctcggccgccttcgctcgggtggcgacttcctctccgtgccccctccccctcgccatgaagaagcccattggaatattaagcccgggagtggctttggggaccgctggaggtgccatgtcttccaagttcttcctaatggctttggccacgtttttctccttcgcccaggttgttatagaagctaattcttggtggtctctaggtatgaataaccctgttcagatgtcagaagtatatatcataggtgcacagcctctctgcagccaactggcaggactttctcaaggacagaagaaactctgccacttgtatcaggaccacatgcagtacattggagaaggtgcgaagacaggcatcaaggaatgccagtaccagttccggcatcggagatggaactgcagcacagtggacaatacttctgtctttggcagggtgatgcaaataggcagccgagagacggccttcacgtacgcggtgagcgcagctggggtggtgaacgccatgagccgagcatgccgggagggcgagctgtctacctgtggctgcagccgcgctgcgcgccccaaggacctgcctcgggactggttgtggggcggctgcggagacaacatcgactatggctaccgcttcgccaaggagttcgtggacgctagagaaagggaacgaatccacgctaagggttcctatgagagcgcacgcatcctcatgaacttacacaacaatgaagcaggccgtaggacagtatacaacctggcagatgtagcctgtaagtgtcatggagtgtctggctcctgtagcctcaagacgtgctggctgcagctggcggacttccggaaggtgggcgatgccctcaaggagaagtatgatagcgcggcggccatgaggctcaacagccggggcaagctggtgcaggtcaacagccgcttcaactccccgaccacgcaggacctggtctacatcgaccccagtccggactactgtgtgcgcaacgagagcactggctcgctgggcacgcagggacgcctgtgcaacaagacctcagaggggatggacggctgcgagctcatgtgctgtgggcgtggctatgaccagtttaagacagtgcagaccgaacgctgtcattgcaagtttcactggtgctgctatgtcaaatgcaagaagtgcacggagattgtggatcagttcgtgtgcaaatagtggtgtgcctgcccttcacccagtcccactcccaggacccacttatttatagaaagtacagtgcttctggttctttttatttctcccccaagaattgcagctggaaccatgtgttttgttttgttttattttgttttttcttttctgttaccatctaagaactctgtggtttattattaatattataattaatatttggcaatagtgggggaaactaagaaaaatatttattttgaggatctttgcaaagttagtacaaaatttctttcttctgatgctacaggataaaggggaaaaactatgtattcgaacttagctgtgcagttgggggttcacatctagaaggtgtaggagccattttcttctcaaacagagagtcctttgagatgggtggtatccaggtgaaggaggaggtacagacccatgaataacagttcctgtgaccaaaatgaattgcaggtgctctggtacaaaagatcttaaatatagatatattaaatatacatatatgccaaaaatacagaatatgagacactccctaacccagaggttaccagcctggttttgtgggttttttgttttgttttgttttttctttttttgggttttgtttgtttgtttgtttgtttgtatttttggtgtgtgtgtgtgtgtatttctagaatgatcttttagaaggtacaagcaagaatctcatatcttcagaagcaggcatatcatgtatgttactgtgtcccacctacagatactccattcatgaatgggcctttttctaacagttcatgaatattggggagccggtgggctgggggagggaggtccccagaaattagaaaacttgaagtttcctacattgaggccataatcttgtgttagcccagctgattcttaataccagacttttagatccataaaggaatttttgactaaaaaaaaaaaatcttgttttgaaagccatcttattttcttaaaaatgaaaaattacccatgaatcccatttgcaacccctcacccccacaggcaacaagaaagtcccatgtagttgagcactgcgaacacctctgtgaggagatgatggcagccatcttcctgcatgatcccatgccctttctggactctctgctggccatgcttccgaatggcagccctggtggacactcactgctggtagggcagaaaatgtacacgaggagccatgttcagaaccagccacttaggggttgttctctgaggcttttctttggaggtacggtaacttgatgtgttttgatgatatctcttggcccagggagtccacagaggtgttgcagctgtttggttgttatcctcctgcgtttagactttccatttgtgcttttcctattaccctgcaggtgtaccctaaaactgttcctagtgtacttgaacagttgcatttataaggggggatgtggtttaatggtgcctgatatctcagtttttttgtatataacatatatataaatatacatatataaatatagatataattatatctcagtgcagtctgggatttagacctacagttttctctgggcttgctctctgcctggagtatcgtccttcattgcagtccaattgggatttctttttttccaaaaattttgagtcttaacattgacctgtgacaggatcctaccacgaataccaggaagcaagctaagactcggaggaagctctcagggctcatgtcctgaatgtatgttggttagaaagtagcctttctgcttcctgcccatggccagttctccaccctctctttggtgttctttgtggggagggcactgtggtttgtcgcagccctggacttcgagaggctcccagaacccaggatcaccagcctcctgtctgtttgcttcactcctttcccagggaggacttgggactgtcctgtctgacaggacggatctgagttcccgaagcaaaccagctcaccacatagatagctagtttaaacaatgttttaaaataagggcacctctgtttcaaaagtgacatctgctgtgttgttttcgaggcctgatactcttacaaggtttgaaaaaaaatgtgtgtatccattcatgggcttggtagccttctggtcacctcagtcctgtggctcttaacttattgcccaacaatattcatttcccctcagctacaatgaattgcaagcaaaagatgttgaaaaaaagcactaatttagtttaaaatgtcactttttggtttttattctacaaaaaccatgaagttctctctctctctctctctctctcttatttgttaaatcagattatgttctttttttgtttttgtttttagtgattcatgtttatgagcagagtggagtttaacaatcctagctttaaaaaaaacctatttaatgtaagatattctacgcatccttcagatattttgtatatcccctatggcctttattctgtacttttaatgtacatatttctgtcttgtgtgatttgtatatttcactggt

SEQ ID NO: 85 - DNA, 2821 bp, Homo sapiens
gatgatgctctgcgttgctgtcatggctgcctccatgcatgtataatctcacatgggagtctaagtttgtgagcgctatccgtcgtaacaagacgccggcgactttggaccctcctcctctcgtttttctcctccttagtgggacactgtatcgggtgcagcgatgccgggaatgatgtggacgccggagctggcgcttctgaggggcttctccacggaggcggagcggctggtttggaagcaggagggcatctgtggttcagatattggagttttcttagaattgctgttagaagggagctatgaggctttgttcttccattcaacgacacaaactattttaaattcaacaatgatggctgaagaaaagattgacagctacctggagaagcagatagtgaatttcctggattgctctacagatttggaagaaattgaaagacagcagctggtattcctacttggtgtgagcagcttgcagctttttgtccagagcaactggactggacctcttgtggacttgcatcctcaggatttcttgccatctggtctgctggagcaattcagtgaggtcaaaggactggatgctattattatgggtctcctcattctagatggcgaatcagtctacagcctcacttcaaagcccatactgctgctaatagcacgcattatcctagtgaacatcagacataaattgacagctctccagagcttgccatggtggacgttgagatatgtgaatattcatcagcaattgcttgaggagcgatcacctcagctttttgctcttgccaaaaactgtattgatcaagtgatgaaacaggagaatttatttgaaggtgattcaggtcgacttttggctattcaatttcatctggaatgtgcacatgtatttttatattattatgaatataaagaagcaaaggaccagttcagtactgctaaagacatcagcaagttagagattgacctgacaggtgccttgggaaaaaggacacgattccaggaaaattacgtggcgcagctgattgtagatgtcaggagaaaagaggctgtccctttcagttgtgagttcagcccagcacccacgccccaggagtgtttggccaagaatcttgagctcaatgatgatactgttctgaatgaaataaagttagcagatagtgaacggtttcagatgccagacctgtgtgcggaggagcttgccgtggtccttggagtctgcacaaatttccagaagaacaacccagttcataagctaacggaggaggagctgctggccttcacatcgtgtttactttcacaaccaaagttctgggccattcagatgtcagccttgatcctccggacaaaacttgagagaggaagcacacggcgagtggagcgggccatgaggcagacccaggctcttgcagaccaattcgaagataaagccacatctgtactggaacgtctgaagattttctattgttgtcaagtaccccctcactgggctgttcagcgccagctcgcaggtttgctgtttgagttgggatgcacctcctcagccctgcagatatttgagaagctggagatgtgggaggatgtggtcatctgccacgagagggctgggcggcacgggaaggcagaagaaatacttaggcaagaactggagaaaaaggaaacaccaggtttatattgcttacttggagatgtcctgcaggaccattcctgctacgacaaggcctgggagctgtcccggcatcgcagtgcacgtgcccagcgctccaaagctctcctccatcttcggaacaaggagtttcgggaatgtgtggagtgcttcgagcgctcagtgaagatcaatcccatgcagctcggggtctggttctctctaggctgcgcctacttggccttggaagactatggtggatcagcaaaggcgtttcagcgttgtgtgaccctagaacccgataatgctgaggcttggaacaatttatctacttcctatattcgattaaagcaaaaagtcaaagcttttagaacgttacaagaagccctcaagtgtaactacgaacactggcaaatctgggaaaactacattctcaccagcaccgatgtcggggagtttggggaagccatcaaagcttaccaccggctcctggacctccgagacaaatacaaagatattcaggtccttaaaattctggtccaggcagtggttaatgatatgaccgaccgaagtggagctgttgcctccagcctcaaaggcaagctgcaggagttgtttggcagaataacttcaagggtgaccaatgacggagaagtctggagactgtatgcccaagtgcatgggaatgggcagagtgagaagccagatgagaacgacaaggcattccagtgtctctcgaaggcgtacaagtgtgacacacagtccagttgttgggagaaagatgctacagcatttaaggaagttgttcaaagagccataggactcgctcatgtggctatgaaatgcgctgagagcaagtccattccccaggaagctgtacaaacactttcttcagttcgactcaatttacgtggcttgttatccaaagcaaagcagaattttacagatgtggtaagtggagaagtctctggagagctcgacagtgaaatagcagctgtgaatgctctggaggcagagctgcaagacctcagcaaccagcttcgaaaccggtactgactgaaaagcagcttgtggcgcagtctcttctgctttctccaattcctccagtacctgtatttctgaaacacaagatacccgtttttaaaataaatttgtttgctggcaaacgcc

SEQ ID NO: 86 - DNA, 2655 bp, Homo sapiens
gacctggagagccaggaagatggtggtgatggcgcgagttccgcgttccgagcggccggaccttgtttttgaggaagaggaccttccctatgaggaagaaatcatgaggaaccagttctccgtcaaatgctggctccgctacatcgagttcaaacagggggcccccaagccccgactcaaccagttgtatgagcgggcactcaaactgcttccctgcagctacaaactctggtaccgatatctgaaggcacgccgggcacaggtgaaacatcgctgtgtgactgatcctgcctatgaagatgtcaacaactgccatgagagggcctttgtgttcatgcacaagatgccacgcctgtggttagactactgccagtttctcatggaccagggaagggtaacacatacccgccgcacctttgaccgtgccctcagggcactgcccatcacacaacactctcgcatctggcccctgtatctacgtttcctgcgctcccacccactgcctgagactgctgtgcgaggctaccgtcgcttcctcaagctgagtcctgagagcgctgaggaatacatcgagtatctcaaatccagtgaccggctagacgaggctgcacagcgcctggccaccgtggtgaatgatgagcgctttgtatccaaggctggtaaatccaactaccagctgtggcacgagctgtgtgaccttatctcccagaatccggacaaggtacagtctctcaatgtggatgccataatccgtggtgggctcacccgtttcactgaccagctgggcaagctgtggtgctctttggcagactactatatccgcagtggccacttcgaaaaggctcgggatgtgtatgaggaggccatccgcacggtgatgaccgtgcgggacttcacccaggtgttcgacagctacgcccagtttgaggagagcatgattgcagcgaagatggagactgcctctgaactggggcgtgaggaggaggacgatgtggacctagagctgcgcctggcccgcttcgaacagctcatcagccgacggcccctgcttctcaatagtgtccttctgcgccagaacccacaccacgtccacgagtggcacaagcgtgtagccctccatcagggccgccctcgggagattatcaacacatacacagaggctgtgcaaacagtagaccccttcaaagccacaggcaagccccacacactgtgggttgcattcgccaagttttacgaagacaatggacagctggatgatgctcgtgtcatcctagagaaggccaccaaggtgaacttcaagcaggtggatgacctggcaagtgtgtggtgccagtgtggggagctagagctcaggcacgagaattacgatgaggccttaaagctgctgcggaaagccacggctttgcctgcccgccgagctgagtactttgatggttcagaacctgtgcagaaccgtgtatataagtcgctgaaggtgtggtccatgcttgccgacttggaggaaagcctgggcactttccagtcaaccaaggccgtgtatgaccgaatcctggacctgcgcatagccacgccacagatcgttattaactatgccatgttcctggaggagcacaagtactttgaggagagcttcaaggcatatgagcgtggcatctcactgttcaagtggcccaatgtttcagacatctggagcacctacctgaccaaattcatctcgcgctatgggggccgaaagctggagcgtgcacgggatctctttgagcaggcactcgatggctgcccaccaaaatatgccaagactttatacctgctctacgcacagctggaggaggagtggggccttgcccgccatgccatggctgtgtatgaccgtgcgaccagggcagtggagccagcacagcagtacgacatgtttaacatctacatcaagcgagctgctgagatctatggcgtcacccacactcgtggcatctaccagaaggccattgaggtgctgtctgatgagcatgcccgtgagatgtgcctgcggtttgcagacatggagtgcaagctcggtgaaatcgaccgcgcccgggccatctacagcttctgctcccagatctgtgatccccggacaactggggcattctggcaaacatggaaggactttgaggttcggcacggcaatgaggataccatcagggagatgctgaggatacggcggagtgtgcaggccacatacaatactcaggtcaacttcatggcttcacagatgctcaaggtgtcgggcagtgccacaggcaccgtgtctgacctggctcctgggcagagtggcatggatgacatgaagctgctggaacagagagcagaacagctggcagctgaggcggagcgggaccagccccccagggcccagagcaagatcttctttgtgaggagtgatgcatccagggaggaattggcagagctggctcagcaggccaaccctgaggagatccagctgggcgaggatgaggacgaggacgagatggacctggagcccaatgaagtccgactggaacagcagagtgtaccagctgctgtgttcgggagcctaaaggaagactgactccctccacactttttcttccatggtcactcccccataactacccttctaatacatcccctctttgt

SEQ ID NO: 87 - DNA, 4131 bp, Homo sapiens
cgggcgccgagcgccgagcgccgagcgccgcccgccgagcgccgcgtcttcccgcgccgccgccccgagctccgcccgccgcgcgctccgcccgccgcgccgccaggctgcccggcggccgcccagagcgaggatgcggcggcgctcgcggatgctgctgtgcttcgcgctgctctgggtcctgggcatcgcctactacatgtactcggggggcggctccgcgctggccgcgggcgggggcggcgccggcaggaagggtgactggaatgacatcgactccattaagaagaaagaccttcaccacagcagaggagatgagaaagcacagggtgtggagaccctccctccaggcaaagtccggtggccagactttaaccaggaggcatacgttggcggtacaatggttcgctctgggcaggacccgtacgcacgcaacaagttcaaccaggtggagagtgacaagctgcacatggacagaggcatccccgacacccggcatgaccagtgtcagcgcaagcagtggcgagtggacctgccagccaccagcgtggtgatcacattccacaatgaagccaggtcggccctgctgcggacggtggtcagtgtcctcaagaggagtccacctcacctcatcaaggagataatcctggtggatgactacagcaacgaccctgaggacggggccctcttggggaaaatcgagaaggtgcgggtcctcagaaatgaccggagagaaggtctaatgcgctctcgggtccggggtgccgacgccgcccaggccaaggtcctgaccttcctagacagtcactgcgagtgtaacgagcgatggctggagccactcttggagcgggttgctgaggacaggacccgagttgtatcccccattatcgatgtcattaacatggataatttccagtatgtgggcgcctctgctgacctcaagggtggttttgactggaacttggtgttcaagtgggattacatgacaccagagcagaggcgatcccggcaggggaacccagttgcccctataaaaactccgatgatcgctggcggcctcttcgtgatggacaagctctattttgaagagctggggaagtacgacatgatgatggacgtgtggggaggagagaacttggagatctcattccgagtgtggcagtgtggtggcagcctggagatcatcccgtgcagccgcgtgggccacgtgttccggaagcagcatccctacacgttccccggtggcagcggcactgtctttgcccggaacacacgccgggcagctgaagtctggatggatgagtacaaacatttctactacgcagcggtgccttctgcacgaaacgtgccctatgggaatattcagagcaggctggagctcaggaaaaagctgggctgcaagcccttcaagtggtacctggacaacgtctacccggagctgagggttccagaccaccaggacatagctttcggggccttacagcagggaaccaactgcctggacaccttgggacacttcgcggatggagttgttggaatttatgagtgtcacaacgctggaggaaaccaggaatgggccttgacaaaggagaagtcggtgaagcacatggacctgtgccttactgtggtggaccgttcgcctgggtcactcatcaggctgcagggctgccgggagaacgacagcagacagaagtgggagcagatcgaaggcaactccaaactgcgacacgtgggcagcaacctgtgcctggatagccgcactgccaagagcggaggcctgagtgtggaggtgtgtggcccggccctgtcacaacagtggaagttctcactcaacctgcagcaataagaggcccccggggcccaacgtccacccgcacaccggcccagtttgttgatcacgtaattacgtttctgaaactttccgcaaaactatatacctcagtgttccatcacggtctgacagtcttaagtctcaagtcttcatcgaggcatgagcggggcagacagcagtgcagaagaggaaaggagcccctggggctcaggacagagggtccctcccaaggctggcgccctctgcctccctctttctgcttcctgctcacaccccagcactggccggaacaagtgggcaagcagcaatcatgtttatatctcagacagaggaagcaggggcaggcgcccccacagtgagagctcgccaatctccactctgttttacctattcaaaatcgtgtgtgtgtgtgtgaaccaggcatggtggccagtgcctgtaatcccatcactcaggaggctgaggcaggcaaggccctgtctaaggaaaaaaaaaaaaatgatttccacaaagctggcctttccttgtctgtccctgcacccgggctcagctggcctcccagccacgtcccctcttccattgacatacttgctgtatttgaacgtgaactttcctctggtggacactaaatagagataaagagaatcatggtccgttccctacgcttccccgacatcatcttgtgaaccctgcaacgcagcgcagcccagagcaaaggtgcatctctggggcctcaccagccggcccacgctgctcctccagtaggggcatccccaaaccactggccccacccacaagcgtcaatgtttaacttcagcgaggggatcgcagctgggttagaaagtctgctttgtgcccattgtcatctgtaaaactaaataggtcacctcttttggcaagtattgctttgggttttcttttaaaattagatttcttttctttaggaaaactcaaccccacagcttctaactaatgtgagccagcaggaccctactgccttgtgacagaccagggacactggacagctgtcacccactatccctaggctcacagtggtttgagaccctgtatgttgggtctttggtactaagctgccggcagagcacagaagagcagctcttcagcctgtgggtggaccccacgtgcccgtagccctggtgtgggtaatacccccggtacatccacaggcaactgcaggaattatgggtagagttcagatcacaaggcctgcgcactcccactgaggacctaggcatgtgttagccacagaggtgacctggatgtggggaagtgacacctggtgggctgcaggcatcatacgtgctcatctcgggtatcccagagatctcgggaatcagagttccaggtggtcagagggcctgtaggtgagtgctgtgcctcccgcctgctccagaaggggggtagctggtttcttctacctgaggggctccatgtgtgtactcaccatgtgtgtattcgccatgaccatggaccctctgcgaaggcctggccaggtgctgaccctctgtctttctacccctgccaagcaaccaacagaagagacattgtaacagacagggtgcccacatgtctaattaagttcatacaatgtccaccttccaatgtaggctctgggaactgtcactcccaggtacaggtgcaaagctgtcaaggacaaggcaggagtttgctggagtccggtcaccacgacctttgctaaccctgacactcctgcactgcttctatcagagtgactccacagatgtgagacaacatggggcgtcccaaccacacgtgtaccccaccccaccctgtggcctcttaacctttgtaccatctgattgtctggccaccagggtgccaactcaacccagctccgagcagaggggggtgtgggcagggcccgtcggtgagccatgtttacatgacagtgtgccaaagtgactcgccgagcttgtattgctttgtagtcatctgggctatcccaccctcccaactcctgtttttaaaactaaaaactcccaggagccctgggcgcacacaggcaaaggtggctgtcttctgtggtggtccttgagtctccgtgaagctgaaaatgatggtgtctgtgagtatgttttgcaaattcaaaatatagtttggtaattttttttccagttgatttttaaaaaaacaaaacaaaaaactgctgtacagagcttgtactttgtccattttatagatggaaaccatccttgaaattgtttaacttaaataaagagaagatactttatagataaaaaaaaaaaaaaaaaaaaaaa

SEQ ID NO: 88 - DNA, 1099 bp, Homo sapiens
ggcaggcgcactggctttaccatggcggagttgatctctttcgtagttcccacccagagcgacaaagttttgttggtgtgggatctgagcactgggcccccagccgaagccttaagtcattctctgttcacagtgttctctcagtttggccttctgtattcagtccgagtcttcccgaacgctgcagtggctcgtcctggtttctacgccatcatcaagttttactcctcgcgggacgcacagagagcccagaaggcttgcgatgggaaacccctttttcagacatcaccagtgaaggttcgtcttggaaccagacacaaggcactgcagcatcaggcctttgctctaaacagctcacgatgccaggaactggccaattactactttggcttcagtggatggtcgaaaaggatcatcaagctgcaggagctctccggactggaggatgcagctctcgctgtgcccatgcagaaggggagcccccagttcctctgcgctgtagaggtggtgctgcccccctacggatgcaggagccctggggttggcatctctgaggagcctctgcgccagctggaggaagggcagtcctcgtttcttatgaagaggaagacagctcagaagcttgccttccaggcggctgtatcagatgccttccagaagctgaccatcgtggttctggaaagtggcaggatcgcggtggagtacaggcccacggcggaggatctagatgccagaagtgaagaagagctgcagaatttaatccaggtcagctgctcctcttggagtcagtccagccaaagggaggaagaatgtctctcggatttcagcctggaggaggaagacctgaagctgtgtgatccgcactagcacctctgaccgtgtcaggtgacctgacctgccttctgactttgacaggaagttctgcgtgtccaatcagtgggttgctgccctccctccctgagcatccaagtgtgaagccttgatcttgctgggctttgtaggagggctctgggggagaggtctaaaggaggcttgagccccgtcactaaggtggaaaaaggttccccatgcaggcgtcccatcctaataaagactacgt

SEQ ID NO: 89 - DNA, 1214 bp, Homo sapiens
ggccggaagtagtgattgctagcgggggggataccttcgctcagcatggcggcgattcccccagacacctggcagccgcccaacgtctacctggagactagcatgggagtcattgtcctggagctatactggaagcatgcgcccaagacctgcaagaacttcgcggagctggctcggcggggctactacaatggcaccaagttccacaggatcatcaaggacttcatgatccaaggcggcgacccgacaggcacaggtcgaggtggcgcatctatttatggcaaacagtttgaagatgagcttcatccagacctgaagttcactggggctgggattctcgccatggccaacgcaggaccagacaccaatggcagccagttctttgtgaccctggcccccacgcaatggctggacggcaagcatactatttttggccgagtgtgccagggtatagggatggtgaatcgagtgggcatggtggaaacaaactcccaggaccgtcctgtggacgacgtgaagattcttaaggcgtacccctctggataggtgggatgctgtcccagcactcccaggccccgggagctggtctccggatgacgtagggtgctgtgtgattctgcgtcatgcatacctactctgaagcagaggggtcgtctctggggacgtttgtattctgccagacagtcacagacttgaaaagcgggccgcgttgatgagcgcactagctgcgcaccccctggtcacagccccgttcagcctccagcgtgagttcttccaggaatatgcacagctcactcgctcttccctctgagcctgagcatcaatactgctgcttctgacactacacatcccacgcaaagccatattgaattgttgccaaatgaaaaatgcatccaacagtcaagtgctgagaaggtgtcaagtggggatgataatgtgtaatgattgggcaatgaatctctgagaggaagcaggaaggagccttggccatttttccccggggagggatgggagtctagtgagcacaaggccgtcacaggcttcccacaggaagtgacatgctttagggctttagagaaaataggtaagaggtttttgtgcatgatgtccttggttttttttttactatttggatttcaagggagtggggcaagaaaggtattaatacctatgtaatacatagaaacttacagaataaaccaccgatgtggttg

SEQ ID NO: 90 - DNA, 3706 bp, Homo sapiens
ggcgggacaacggtcggcgacgcagcttgtggtgggagcggaagagctctcccggcgggtctctgtgcagttggcgaggcagagggccgccgcagcaccatggcgaccaccgtcagcacgcagcgcgggccggtgtacattggtgaactcccccaggacttcctccgtataacacccacacaacagcagcagcagatccagctggatgcccaggcagcccagcagctgcagtatggaggaacagttggtaccgtgggtcgcctcagcatcactgtggtacaggcaaaattggcaaagaattatggcatgactcgtatggacccttactgccgtctgcgtctgggctatgctgtttatgaaactcccacagcacacaatggggccaagaaccctcgctggaacaaagtcattcagtgcacagtgcccccaggtgtggactcgttctaccttgagatcttcgatgagcgcgccttctccatggatgaccgcatagcttggacccacatcaccatccccgagtccctgaagcagggccaagtggaggacgagtggtatagcctgagtgggaggcagggcgatgacaaggagggcatgattaacctggtcatgtcctacacgtccctcccagctgccatgatgatgcctcctcagcctgtggtcctgatgccaactgtgtaccagcagggtgttggctatgtgcccattgcagggatgcctgctgtctgcagccctggcatggtgccgatggccatgcccccaccagctgtggctcctcagccccgctgtaatgaagaggacctcaaagctatccaggacatgtttcccaacatggacagggaagtaatccgttctgtgcttgaagcccagagagggaacaaagatgctgccatcaattccttgctgcaaatgggtgaagagtcctagacccgcctgtggcccttggcttcacaccactgacccttggacttgtccacccagggctccccagggtcaccatccccacaagattccaatgaaagaccatccacataccccttccttggacatctgcgccgccccttcctacatatttcggggatgcatgtggatttttggttctaagcaggcagtcctcttcattgttgctgggggacagagttaggggacagttctctcctagtctctagctgggtgatggtggcatggccctgttattgcacagatcagtggtcctgtttggggacctgcatctcctgtcctcaccctattctggaaagcattcttacagaaagcaactgcctctgccaagatactagaaattgtacagcatccttctgagttgatggatggatggcatggagttactcagtccagctgggatggttgttgagggtgcactgagttcatgtgcacttacttacaggttatttttagaactttcaagctaaaagctccttccctcttctgatgttgtatgtgaacatgggcccctcagctagtcctggccagtgagatatgcttcccagccatatccagtgggtctctaacctcctgtctagcttatctgccactgtctggctaggcttcagccacagaaatccaacttgaatacagaaggtatccagacctggtgcttggttggccaggccccacattagccctcactgctggaggctgctttccacatcctcttgtttagtaccagcatcatgggtcttactccaggggctgttcttgttgatagaccttcatcacagagcccagagtcaagcgaaggagatgggtgggtgtggcactatgtgcttactactaaaattcaagcattgtgacactactgacctgcagtatactctgtggacccatactgtagcttggcttgagtgataaaaattcatgcttaaattaaactttcataaaggtagaggtgcctgtacctgaccagtggatattgtatagtgtattttaggttgcaaacctttgagcccgtgacctggccatacatgtatttagggagaaattgagtggtgccttgtgctaagatacagaccgtgaaagctttctcagaaccagtactgtcacaaggtgctgttaggaagctccagcattgccacagggttatggtgggacagttttgccttacattgccgttactttttgttttctgagaatctgcacacccggtttggatatgtgatccaccaataagggcatgaaggtgactcctagcagtttggactgttgtgaaggacttcaaataagaaaattagtgcttcagtttttggtctttgcctcctaaaaacgcggtaaaatatcctacttgctgttatctacaggggtgctttttgtcaaatgtattgtttataagtatttatttttgtaagcctaaaggcagtatgtggtgatggtgagcatgggatggtggtaacaagcacttttctttagttgtaggtctttaaaccttttaactgcaaaatggagaagaaagaccaatgtttattaaatagattcaaccacttactttaacacaaccaggcactggaagcctccattctagactgttgatcggccttgtgcttggtaagcagatgccctcttgcagggtggatgagtcaggcaggcagaacctccttaagtggtccacttagtgttaccagcagactgcagacactgcagagcaggaacctcactcagtacctgtctctaggtctcaggtgtggcggctgtgtgcatccgcatctgccttacggaggtataggttgaacacagggccactttgttctgtgaggttctataggaagcatcacgatttgctgccagcccctgagcagtcccacagcactcagaggcagcgttcactaacttacgactgcttaaatgaggaagagtgatggttttaagacagatttaagacttgagctgaacacagactcttccctttgtcatgaacattttatttttttaatagattgaagaaagatcacattttataaggcctgagtgatactcctaggttttgctgtttttttgtcttggaatttcatgtcagcgcccatctagtgcaaggagagaggcagcacttgtcagcgcctgtgtaggtaccagggcatcagagcatggcttgttggcttccatcccatcctgaatcttgttgagatgaagagtcatccctagaaggagctcagaggcagaggccacagccgagtcagacaggcttctgcatggggaccaggctctcactgggtgtaattgctgctcttgccaagtttgaagattgatgttggtagcaaaatttctcaaggtgttcactggagccccctcccctcgggccagctctagtgtaggcatgcatctagggatggcctggcctgttttcttctctggacatgttggttggctgaagttgtaacctgctgcacgcttgtgggctgacatggccagaggctttcatctcaccatgtgagtgatgcctacccgatgacagaagtgttgtgcgactgggctctgccctcgccctcccaggccaccctttacagacactggtatcatctggtacactgtctggaggttggtgggaatggttctcagaactgtctgtgattattttgggaatgggggggtccatgtccatatttttctttgattatatgatatatttttacttttcagccttctaatttaataagtggtctatataaaatagaaaaataaagtcctctgaggaaatgtt

SEQ ID NO: 91 - DNA, 3847 bp, Homo sapiens
gcggcccagcccgccacggaggcgcgcaggcgggcgcgcggagtggccgctcaggagtatctctttaccacccccaagaaactactcaatgctgaagcccttcaaagtgatagcggagactttggaagcaccatggatgggttttgtgatcagcaagtcccttttatggtcccagggaaatctcgatcagaggactgtcgagggcgacctttgattgacagaaagaggaagtttgtggacacagatctggctcacgattctgaagagttgtttcaggatcttagccagctgcaggaggcttggttagctgaagcacaagttcctgatgatgagcagtttgtcccagattttcagtctgataacttggtgcttcatgctccacctcccaccaagatcaaacgggagctgcacagcccctcctccgagttgtcgtcctgtagccacgagcaggctcttggtgctaagtatggagaaaagtgcctctacaactattgtgcctacgataggaagcctccctctgggttcaagccattaaccccgcccgccactcctctgtcacccacccatcagaattccctatttcccccacctcaggcaactctgcccacctcaggtctcacccctggagctggcccagttcaaggggtgggtcctgccccgactcctcactcacttccagaacctggatcacagcaacaaacatttgcggtcccccggccaccacatcagcccctgcaaatgccaaagatgatgcctgaaagccagtatccatcagaacagagatttcagcgacagctgtctgagcccagccatcccttccctcctcagtcaggagttcctggagatagtcgccccagctaccaccggcagatgtcagagcccattgtccccgcagctccgccaccccttcagggattcaagcaggaataccatgacccactgtatgaacatggggtccctgggatgcctggtcctccagcacatgggttccagtcacccatggggatcaagcaggagccccgagattactgtgctgactcagaagtgcctaactgccagtcatcctacatgagaggcgggtatttctccagcagccatgaaggattcccgtatgagaaggaccccaggctgtactttgatgatacttgcgtggtacccgagagactggaaggcaaagtcaagcaggagcccaccatgtatcgagaggggcctccttatcagagacgaggctctctgcagctgtggcagttccttgtcaccctcctggatgacccagccaatgcccacttcatcgcctggactggccgaggcatggaatttaagctcatagaaccggaagaggttgctcgccgttggggtatccagaagaatcggccagccatgaactatgacaagctgagccgctctctccgctattactatgaaaagggcatcatgcagaaggtggctggggaacgctacgtctacaaatttgtgtgtgacccggatgcactcttctctatggccttcccggacaatcagcgcccgttcctgaaggcagaatccgaatgccccctcaatgaggaggacacgctgcctctgactcactttgaagacaaccctgcttacctcctggacatggatcgctgcagcagcctcccctacaccgaaggcttcgcttactaagtttctgaatggccgagtagccacaccctagagctagcagtcctcatccaggcaaccgagggcagtggttttgtttgtgttcttggctgttcctaaagcttgccctttgagtattatcaggagaacccaagctgtctctggattcgacaccctttaagacaaataccttggctcgggaggagtgggaacagggagagacaaaaaccaccacttcctggggagacgtggataccgtcctccttgccaccatggccagtatgtgcagagcagtactgcagtgtgttcagggttagcgcccatgaaaggggaccgagtgtatgacaatctgcgttgatcatggactactaaaatgccttcacacaggaggctttgacttgcacaattctttgggaagaaactgcacacccataggacagtcgataaggctgagtcggggaggctcagaccattgaggcttgaaattgcaaaactcttgtagctcattctgaaatctttcccctgtagtccagaactggggaggccaggagagccgttactcactccatggctctcagaccgcgttcctctttggccttccttccagccgagccgtcaccataagtaaatcaagtcccttcctcccagtcacacggctgagcgcagacggcttgttttaaaaagatgatgaacatgacagtattttgttttgctttgatttgggggggaagggttcatcttgcttttggaggttgttttggggaaaaaacagtttatataaactgatttttgtagttttggtatttaaagcaaaaacaaacaaaaaacaaacccctttttggtacctgcactgcatccctcgggtggggcagtgcctactttggaagatgctgcagcctgcgaggggatctgctggggcttccccttcgccacacagaagcctgcttagtgctttctgcaccagacaacctgttgaggcgtataacctgatctgtacatttttttaagtgtgcagggcagcctggacacagcttagcgttctacgtgtatagttctccatgctcactaactctccttctctgggaagcagatgtacatactacatacatgtcacatactacatacatgtcatgtactacacacatgtcatatagtacatacatgtcatgtccatttaacgactggtcacctgctggggacccaggcatctgccttgttttcctttttctttcagttttggactttggaccctgcacagccgttccccagggtctgaaacttacctgtcacgcacaggtagagcagtgctagctagcattactggcttcctgctactttttcgtttttttaaattttttattctgattgctgtattatattggggatgttctaaacaaccaaactaaagctgtgtacagattgggtttggagcggtggggacgtcagcaggaaacgccagtgctttgctgcctgattgacaggaatttgctgtccttgacactccacacattgtgttcctcacgtgtgggacaggacacagagctgactgagcgtgggacactccacagccttctcatgacttgtgaaggaggttcacaggaacgaggggtatgttttagagctatgagaacctagtcagcccacagaggcgtcaaacctgaagcttgggccttaattagcattgtactctgatgaaaggactctttcataagcatatttatagctttctaatctatacacagtctatccatagatgcatattttaccccaactggctagagatttatttgttgtaaatgctgtatagattttgggttttttttttttgttcttcctctcttcacttatactggtgtggggtttgttttagttttctttagtgaatttatgctgttttggggggcggattgagaagaatcttccgtagcctgagctgtccctcctgttttgacggcacatgtgccagtcaagggacatgggacagtcgcgccactttgcattcccacgggactcatgtacctcccgcagagccgtccccgtcaaggtgtggggctcctttcatgcatttgcttcttttccagagtgtggatttgaactacttccccctgcaaagggccgcgtgcaccctgccgagctgcgtatattctgtactgtgtacaataaagaagattgctttccttaaaaaaaaaaaaaaaaaaaaaaaaaa

SEQ ID NO: 92 - DNA, 3664 bp, Homo sapiens
cttttctctccgagcaagatggctgggggtgagggtggagtgactctcggtcagccccatctttcccgacaggatctcgccacattggatgttaccaagttgacaccactttcacatgaagttatcagcagacaagctacaattaatattggtacaattggtcacgtagctcatgggaagtccactgtggtgaaggctatttctggagttcacactgtcagattcaaaaatgaactcgaaaggaacattaccatcaagcttggatatgctaatgctaagatttataaacttgatgacccaagttgtcctagaccagaatgttacagatcgtgtggaagcagcacaccagatgagtttcctacagacattccggggaccaaagggaacttcaagttagtcaggcatgtttcctttgttgactgtcctggccatgatattttgatggctacgatgctgaacggtgcagcagtgatggatgcggcccttctgttgatagctggtaacgaatcatgccctcagccccagacttccgaacacctggctgccatagaaatcatgaaactcaagcatattttgatcttacagaataaaattgatctggtaaaagaaagtcaggctaaggaacaatatgagcaaatcctcgcatttgtgcaaggaaccgtagcagaaggggcaccaattattccaatttctgctcaactgaagtacaatattgaagttgtctgtgagtatatagtgaagaaaataccagtgcccccaagagactttacttcagagccccgtctcattgtcatcaggtcatttgatgtcaacaaacctggctgtgaagtcgatgaccttaagggaggtgtagctggtggtagtatcctaaaaggagtgttaaaggtaggccaggagatagaagtgagacccggtattgtctccaaagacagtgaaggaaaactcatgtgtaaaccaatcttttccaaaattgtgtcactttttgcggaacataatgatcttcaatatgctgccccgggtggtctcattggagttggaacaaagatcgaccccaccttgtgccgagctgacagaatggtgggacaagtgctcggtgcagttggagctttacctgaaatattcaccgaactggagatttcctatttcctgcttagacgtcttctgggtgtacgtactgaaggagacaagaaagcagcaaaggtccaaaagctgtctaagaatgaagtgcttatggtcaacataggctccctgtcgacaggaggaagagttagtgcagtcaaggctgatttgggcaaaattgttttgaccaatcccgtgtgcacagaagtaggagaaaaaattgcccttagcagaagagttgagaaacactggcgtttaattggttggggtcagataagaagaggagtgaccattaagccaactgtagatgatgactgaagacttcaagttaaacaatgccttgggaaggagttggaatttcttttactagcctaggggtatattttcaaagcaatactagaaagaattaatttcctagctcattaccttactagtagcagctataaatttattcttatattttggcgatgaaaatgtgacctctacttttttttttctttttattgattccttgtttgacattatgtagcccaatcccattcatcccctcatatccaacctttgcccatgcgacctcccccaaagtagcacaccaacaacagaacaaagcctagaaaacatgccatcgtggaagctgtagcttctgtgacaacaacaatgttggctcctcatcaggactccctccagttatcctgttgctgcgctgtgtcatagagatcctgcagctttggaacagcagggtggccctttcacgatccccaccattcgaacatgacatagattttaggtgggtcaattcagagccccggataagggccaggatggtagctgagctggtcagctggggcccattcttccaagtgctgcggcttgtgagagcctgggccaattctccctacagaaggtggcaacgggggagggtgtcccatccacaccacctcataacagacaagtggcagggccagctgtccagagtgctatatccaatgagggatggtggcagacctgcacagcctcagacacccagtgttccctagtggtaataatgagccatggacactgacccaccactgtggatccacagactcaaacaaggccctgatggcagggatgcccacttataacaggctacttctctccacccttgaattcctttttctctcccgtctgtcatggtgacaagcaggtatctatgcttatgcactggagggcaggactgtgggtggcatggaggtctacaagaccttgtgttcctcttctcatgccgtgctgccttgctttaatttgatttttatgagtcctaggcataagacaaggtttggccatcaagctaggatgaacaaaggactgccatctgctctgcccctgataaagaacaataccaccaataaggggtctctgctggttgctggggttgtaacctacttctaatatggggcttaaaattaaatgtgaaaagtggtatactatatacattggatcaagtctatatagtagctaaaattaagcattccaaagtaatgtctaatttatcaatcaatgaaagtttgaagtgaaacttggctgcaaccagtctttggcgtagcacttgtatcatgaacctgtctaagatgaaaatgatctcttctcatgattcatctgtgagtagctccatagtgaagggcaattagtaaaggctaaagacttacccctttatttgtccttctatttggggttctgttgtctgaaatttgaaggaatacgaccagtgggctcaaatgagcaccactcttagaagttgtgtgcccattcccttaacacaggtagatctgtcttacccgtttcctgggtgggaccttaggaatccagtgccctgagactatgtctttattatttatgcctagtatctcagtcttcccacactggcaaagagtatagcaactggtggttagtgataattgacaaggttctgtccaggctgcttttttgaggtctttgttttagtaagctatgaatggagctgtctcttgcatgatgaataaacactctaccattgtctccactgcccctagctgtagtactggctgataggattatggtggaaattgtgttattggtaatctttgagccttgagagaggtagaaattgttcagaaactgtgtcctctggtttctaaatctattccccacgtgcctccaattttcttgtgctgtgcattttaaattaatcatgttttttcttaattaaatactctttcacagcaatgatttccaactctggctgacgtaagaattgctggggaagtttgaaggggggaaaaagaaagctctggggtggagtccaggctttgatagttttttttgtttgtttgtttgtttgttttttttttttgaggaaacctattgatatatgtatttgattgactctaaaaagttgttgtattggaacatgaagaaaacatttaaataaaacaagattctgaaacaagaaaaaaaaaaaaaaaaaaaaaaaaaaaaaaaaaaaaaaaaaaaaaaaaaaaaaaaaaaaaaaaaaaaaaaaaaaaaa

SEQ ID NO: 93 - DNA, 2856 bp, Homo sapiens
gcgcactggtaccgacttgccgcgcgccgccgctcctctttccttcctcttccgagcacgtcctgctccgtgtacctctcgggtccctcgccatggctgcgctcacccggaacccgcagttccagaagctcctggagtggcaccgcgcgaactctgccaacctcaagctgcgcgaactttttgaggcggatccggagcgcttcaacaacttcagcttgaacctcaacaccaaccatgggcatattctggtggactactccaagaaccttgtgaacaaggaggtgatgcagatgctggtggagctggccaagtccagaggcgtggaggctgcacgggacaacatgttcagtggttccaagatcaactacaccgagaatcgggcggtgctgcatgtggcccttcggaaccggtccaacacacccatcaaggtggacggcaaagatgtgatgccggaggtgaacagggttctggacaagatgaagtctttctgccagcgggtccggagtggtgactggaaagggtacactggcaaatccatcacggacatcatcaacatcggcatcgggggctctgacctgggacccctcatggtgactgaagctctcaagccttactcgaaaggaggtccccgtgtctggtttgtctctaacattgatgggacccacattgccaaaacactggccagcttgtcccctgagacttccctctttataatcgcctccaagaccttcaccacccaggagaccatcaccaatgcagagacagcaaaggagtggtttctcgaagcggccaaggatccatctgcagttgcaaagcactttgtcgccctgtctacgaacacggccaaagtgaaagagtttggaattgaccctcaaaacatgttcgagttctgggattgggtaggtggccgctattcgctgtggtcagccattggactttccattgctctgcatgtaggttttgaccacttcgagcagctgctgtccggggctcactggatggaccagcacttcctcaagacgcccctggagaagaatgcccccgtcctgctggctctactgggcatctggtacatcaactgctacggctgtgagacccacgccttgctgccctatgaccagtacatgcaccgctttgctgcctatttccagcagggtgacatggagtccaacggaaagtacatcaccaagtccggggcccgtgtggaccaccagacaggccccatcgtgtggggggaaccagggaccaatggtcaacatgcattctaccagctcatccaccaaggcaccaagatgataccctgtgactttctcatccctgtccagacccagcaccccatacggaaaggtctgcatcacaagatcctcctggctaacttcttggcccagactgaggccctgatgaaggggaagttgcctgaagaggccaggaaggagctccaggctgccggaaagagcccagaagacttggagaaactcttgccacacaaggtctttgaaggaaaccgcccgaccaactctattgtgtttaccaagctgacacccttcattctgggggccttgattgccatgtatgagcacaagatctttgttcagggcatcatgtgggacatcaacagcttcgaccagtggggagtggagctggggaagcagctggccaagaaaattgagccggagctggagggcagctctgctgtgacctcccatgattcctccactaacggactgatcagcttcatcaagcaacagcgggacaccaaactagaataactccagccgcggccctactgactggtcctccgtgtcccttctcaccatatgcactgcatggtcctgcccctccctgcccagagcgcaccaccggtagttggcctggactacaaggctgttgggagaagctggtctggaactgccatccacccactacgcaccctccctgttgaagctgatggaagggctttgacgtgtcatgttgttctgacctgtatttcacaccccagctagaataaagacacctagaggaggcatgggggctcagcctcttcagtttactgagtcagtacttttatctgttacttgtgggtgaccctgcttatgccttgtttggttggctctttacttggtggtgggctggtccacagtctcggaggattgatcgttatgaggggacagctgggtgataggggcacttgggaggcacaggcaggtctatctgaattcaaggccagcctggtccacaaaatgagttcccggatagccagggctacacagagaaaccctgtctacaaaaaaggggttgggtagggtggggtcagggatctgcagttaggtgggcaacttgctctccagtcacgtgttcacctccttcccctcctcaccttggtgacgtggaaaaagtaattctgcttcactaggtaggtttttgcttgcttgtttgtttgtttaccttcgctttatttttggtatcaaaacctaggaatggggctggagagatggctcaacagttaagagtactgactgctcttccagaggtcctgagttcaattcccagcaaccacatggtggctcacaaccacctgtaatgagatccgatgccctcttctggtgtgtctgaggacagctacaatgtactcacatacataaaataaataagtcttaaaaacaaacaaaagaacccagggatgaactcgattatacccttcaaaagacagttttagcagggtgtcattactgtcacattcatatcctggctatcagtgagtccttgtgagactgagtgacagccctgtcattctactttccaccatgttaattaggcccatttgatggccagtcaataaaaagacttggtacatgt

SEQ ID NO: 94 - DNA, 1568 bp, Homo sapiens
ccaggcaggggtcgccgcagcctggatgcgccctgtggcgcgcgcgcacgcagcatcccgagcctcccgccgcgcggcggggatgcctgctctccgagccccggggctgggcccccggcggtaaccggagcgggggggcgcgcccccccagcagcagctgccgcgcccgcgcccgggccagtcgccgccggggcccatcgcctgtcgccgcgctctgcgcacccaccgccttgcgcggccatggggacgctgctggctctcgtggtgggcgcggcgctggtatcctcagcctgggggggctgcgtggaggtggattccgataccgaggctgtgtatgggatgaccttcaaaatcctgtgcatctcctgtaagcgtcgtagtgagaccaccgccgaaaccttcacggagtggaccttccgccagaaggggacagaggaatttgtcaagatcctacgctatgagaatgaggtgctgcagctggaggaagatgagcgctttgagggccgagtggtgtggaacggtagtcggggcaccaaggacctgcaggacctgtccatcttcatcaccaacgtcacctacaaccactctggcgactacgaatgtcacgtctaccgtctcctcttctttgataattatgagcacaacaccagcgtcgtcaagaagatccacctggaggtggtggacaaggccaacagagatatggcatccatcgtgtcagagatcatgatgtacgtgctcattgtggtgttgaccatatggcttgtggcagagatggtgtactgctacaagaagattgctgctgctacggaggctgctgcacaagagaacgcctcagaatacctggccattacatccgagagcaaagaaaactgtacaggcgtccaggtggctgaatagcgctggctctgggctccgcctcatggaagagccagcccctacgggtaccctcccgccctgcagtggggatcagcccctggtgggtaccctcccccggcagtggggatcagcccttcggtctccccagcctcacagttctgcagtggagccacgagggtgggagcgggcagggactgatcccacctcacccaccgcctcccacctaccctcccaccgccatgcatgatgggtgaagcaatatggccgccccaccctgcttttgctgcctgtttgggggagggggcggtgaggcgagggggcaggccccgccccttctttttgctgatttgcacataggccacttcccacacgcactgccaggccagccggccccacccctgcttgatggggtcaagaggggtcgggacagggacagtagtgggcaggaggttctgggcctcatctcccctccgcttcctccggctggacctggggttcccttcctgtgacacctcctagccctggcccacccgccctctctcaccagccttcaattgtggtctcttgggaagggcctcttcggcctcctctctttacagaagtagtttttgttcatgaaataaagattcttggactcgaaaaaaaaaaaaaaaaaaaaaaaaaaaaaaaaaaaaaaaaaaaaaaaaaaaaaa

SEQ ID NO: 95 - DNA, 2639 bp, Homo sapiens
ggaaggggccggggcagctccggggaagggaagggctggagctacggcgtccggcggccgagaggggcggtgggctgggtgcgccgcggagcccggcccgagtgtcccgtgcagcgtccccgggtgccgccatgacgcccgatctgctcaacttcaagaaaggatggatgtcaatcttggatgagcctggagagtggaaaaagcattggtttgtgctgacggattcaagcctcaaatattacagagactccacggctgaggaggcagatgagctggacggtgagatcgacctgcggtcctgtactgacgtcacagagtatgcggtccagcgcaactatggcttccagatccacaccaaggacgccgtttataccctgtcggccatgacctcgggcatccggcggaactggattgaggctctcaggaaaactgtgcggccaacttcagccccagatgtcaccaagctctccgactgcaataaagaaaacacgctgcatggctatggcacccagaagagctccctgaagataggggagcagcggacaggctctgaggttatcggccggggtggtcctcgcaaggcggatggaccacggccgtcactggactacgtggagctctctccattagcgccaagctccccacagcgcatgcgcactctctcccgctccactcccgagcgccccaccaagcaggaggatctggagcgggacttggcccaacgctctgaggaaagacgcaagtggttcgagtccacggacggcaggaccccagagacgcccagtggcgatgggtctcgaaggggcctgggtgcccccctgactgatgaccagcagagcaggctcagtgaggagattgagaagaagtggcaggagttggaaaagctgcccctgcgggagaacaagcgagtgccactcaccgccctgctcaaccaagcccacaacgatcgtcggggacctacaagtgacagccatgaggccctggagaaggaggtccagtctctccgtgcccagctggaggcttggcgtctccgaggggaagctcctcagaatgcaccgagactccaggaggacagccatatccccccaggctacatctcgcaggaagcctgtgagcgcagcctggcagagatggagtcctcacaccagcaggtgatggagcagctgcagcggcaccatgagcgagagctgcagcggctgcagcaggagaaggagtggctcctggccgaggagacggctgccacggcctcagctattgaagccatgaagaaggcgtatcaagaagagctaagccgggagctgagcaaaacccggagtctccagcagggcccagagagccttcggaagcaacaccagctggacatggaggcgctaaagcaggagctgcaggtgctatctgagaggtactcgcagaagtgcctggagatcggggccctgacaaggcaggcagaagagcgtgagcacaccctgcggcgctgccagcaagagggccaggagctgctgcgccacaaccaggagctgcactcccacctgtccgaggagatcgaccgtctgcgcagcttcatcgcctcgcagggcacaggcaacagctgcgggcgcagcaatgagaggagttcctgtgagctggaggtgctgctgcgagtgaaggagaatgaactccagtacctgaagaaggaggtgcagtgtctccgagatgagctccaggtgatacagaaggacaagcgcttcacggggaagtaccaggatgtgtacgtggagctgaaccacatcaagactcggtcggagcgtgagatcgagcagctgaaggagcacctgcgcctggccatggctgccctgcaggagaaggaggctgtgaggaacagcttggctgagtagaggtccctgtggttccgatcagctgaggaccctccagccccaggggaccagccaccctccttctctggcagaagtcagaagccaagctttctccccaccctgggtcacatgtgcactcacaccacacacacccacacacacacgcacacatacacacacacacacacacacacgcatatgcacacacatgcacacacacatacacgcacacacatgcacagacactgcggatctgagctcgtcccttgcacagggtctttaccttgcaccttcttcaggattttatatgtgaagagatttttatatagatttttttttttgcctttttttttttctaaaacatttaaactttttaaaaaggcaatttcttggtggcgtgtgcctcaagcactggcttctctccctcttgtcccctgcttggccctccttcctacctgggtgtctcacaaaggcccaaggagagccagggtgagtcaaccagagggaaaaggcagccctcagttccctgccctggcctctgggcagagggaggagccagtggctggccccatatccactcctctgaggggcgctgggcgtgggccaccagcgccaccagcgccacccatgaagacacttctcttctgttccgtgagtcaccctaacttaataaaaccttccggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 96 - DNA, 5869 bp, Homo sapiens
tggggtcagccgccgcaacccgggggaggaggttcccactttaagaagtgaagttttcctccccctccccctcctgcccacctcctgccgcctcccgcgccccgcggggccgcggatggagctccgccgtggcggcgtggggaaccaggctgcgggccggaggatggatggggactgccgcgatggcggctgcggcagcaaggacgccgggtcggaggactacgagaacctaccgaccagcgcctcggtgtccacccacatgacagcgggagcgatggccgggattctggagcactccatcatgtacccggtggactccgtgaagacacggatgcagagtttgaatccagatcccaaagcccggtatacaagcatctatggcgccctcaagaggatcatgcacactgaaggcttctggaggcccctgcggggcctgaacgtgatgatgatgggtgcagggcccgcgcacgccatgtattttgcctgctatgaaaacatgaaaaggactttaaatgacgttttcagccaccaaggaaacagccatctagctaatggtattttgaaagcgtttgtctggagctgggaagctcttctctctggggcttcctcccccggacccagcaaccttcatcctcggcaaacagagaactctagggtcacgtgaatgcggtcacgtgaacgcgcgctttttttttttttttctttcaattcaatccctctttctctgggggaagctggtggtagatgtgttttggtatttggtatttattattgtgtggcttttttttttttcttttaagaaaacactcgaaacaattaatgatcagtcataggaaaaaatatcttttgaatgctggatgcaaaccaaaaatacacagaaaaggacagcttctttgagactatttaaaaactggtacagcaggtatctccagcgccaggatctaactaagctttgaaaggtcaagacgttttatgctgacagaggactccgagccactgtggaaggccatccccactttaagctgccggagactcgggcctttgacccccccccacagcgtcctccgtgtgtctgagtcgcatctttctgcaagccagggctgaactagtttcctgtggttgatctagggagggcgggtggaccgcaatggggcacttgtggacagcttttacataaggcaggagtcgatgggctgggactttaacaggcttttcagtgggtgggagaggaccgtcgatagcagcaaagcaacttaaaataatgtttattttgacaaaatttactctgaggaatggagaagtggatgtgatcaactctcccctttgctttttattcgacccacaataaaaacggtttcccttctgtcagccacccttagacccaagcatacccgggtgctttgctgaagtcctttgctttttcatttggtgaccattctttgaatttattcctttggtcaccaagagccatgcccccatgcttctgagatggtggtaacagaccctcatttcacccaaactaccttacgtaatggcttggctcctttaaggaggaggctaggggacttgacaaaacccatccctgcaccagggtctgcccaatgctgtccctgctgcattggtgtaaatgctgtggtgtctgccacctttctgggaataattgtgtgtgtgtctatgtgtgcactaagtaagaggtgccttcctgagaggtggcctgtagtctgacgctcatccctccctctttccacctgttataattaacgggcagctgcattgctgtggaagtcactgggaggccagaagcagaaattccagggccaattcaggctttcaaagccaggctaaaggagacttcagatgaaattcttcacttacaaggctggtcctaagtctgcttagacttcagctaaggcaactagccttctgcatgattgtatcctcttcagtagccttagcttaagggcccagagagctctctgtacctaaaggcttgggacaagggaataaaattctaggcagtgggaaatgcctgaggtatggtttctgagctctaacttttcccaaggttgatttacaaacaaaaggagtctctgaggtttagctgcaacggtgctccagatagctgctctccctattgctgttcagcgtttctgccaccactttgggattttaaaattttctcaccttggctgagctgctaggcaactttgagccttagaaagctaagtccagagtttatctgacttaagttcatggaacttcccatgagcaccaggtgacagcgggtaattttctggagtttgttttgcaggggtagctgggagtatggccaccctactccacgatgcagtaatgaatccagcagaagtggtgaaacagcgcttacagatgtacaactcccagcaccagtcagccttcagttgtatccggacagtgtggcggaccgaggggttgggggccttctacaggagttacaccacacagctgaccatgaatatccccttccagtcaattcacttcatcacctatgagtttctgcaggagcaagtcaaccctcgccgggactacaacccacagtctcacatcatctcaggaggcctggccggggcactggccgcagctgccaccaccccgctggacgtctgcaaaaccctcctcaacacgcaggagaacatggctctctccctggccaacgtcagcggccggctgtcgggcatggccaatgccttccggacggtgtaccagctcaacggccttgccggctatttcaaaggcatccaggctcgagtcatctaccagatgccttccaccgccatctcctggtccgtttatgagttcttcaagtacatccttacaaagaggcagctggagaatcgaactctgtactaaaggaatggggcagtgggaagtggttctgacatcctttgttccagcttcatcctcttgttctcgcacccagggcttgtcctgaagggtgcttgcgccaggcccttcgttccctggtgccgtagagagggaggaccggcctccactgaccagcatgctgtctgtggggacatccgaggtggtggtaggcgggagaaacttggggagagggagaagttgcttttctcctccttcctccagaggaatgtagcttttctgcctcactgtggccttctccctagatctttagatcacaccgcagaggcaaacagtgacacagcaaagaaaaagtttatgtacataaaagtaccattaccccagtctgaaataggtggataatgttatcctttatttttctacatagagatttttgaaattgtgtgtgcttgtgtgtgtctataaataatattagagttgggaatgatggacctgttttgtttttttgttttttaaataagagagttccattcttccatcatgagaagtaaaacaaacaacaacaacaacaaacaaggggaaaaaggcaccaaagtaataaatggctttatgtggcctgaaattgtgtgtagcgcccctcccatgggagtttaacttgaatgtaaaataataaatataaagtttatattttgaatttctcttttcatggggaaaaaggtacgttgaaaacaaatcaaaatagtctatcacttggagcttttttttgttttgtttttaattttaattttaaatacagttaccctgcgtgcctttctgtcttttctaagacttccaggaccaaaattagggtcctggagcattttctccctctgtagacacccccgtacagtcctcagcaccatggctgtctcagtagaataagaatggctggccttaaccttccgtgtgagtgatgtgagcgtgtgcctccctgttcagctcttagcactgggaatgccactgtatgcctagccaattgatgagacagttcctgcctttgtcttaggagtaatctccatagcaacctcctccttgggcacttctgctaacctggtctataaggtttgttagtagtctcctgttgacccatttccacggcagtctagggattttgagtatttctggtactcatctgtcacctaccctgttcagctccacgacttaaacatcagagcctcttcactgcactgaggccagccctccagccgggactgaagcctgccacgatcctccgcccctcccgagctgctcagcacgcttgtctcaccctgtgtcacttgagaacctccgtcctttccgctttgttttctgaagtggagtcagatcatctcagagaaagcacaagcctgtgttgtccagtctcccatcctcatggagatgacaggataggctcagtttccttcttccagttgtcaacgagtaagctgggaagacgctcatgttttcgtgtccattgccctggtctcatttgggagccacgactagcatgctccctcactggggaggcttagaccttgagttcccacggatggtcatgctgctgcctgggaagaaattgagcttgtacttggagaatttcatcttggacccacgtaccacttggtgccagtagatgcagctttttggttttgtccagcagctatgagaagttacacactggtcagctgactttttttttttccccagagagaaccacctctctcaagtactcctatgtattccaggagggcttcctggctggctgcagtttgtcttactagtgtcctgggctggctggtgtcctattatcactgacaatgtttctattcttaccctgctagcattgagtagagttaaattctataaatggagagggaagtcaaattgtagagtctaaagtcaaatttaaatcaaaccccaaactattcctataccacgcttagatacattgttgactaatcctgctgcaagaccaatattttcccttggattttctttacagttatctaatactaggatagcccctttggtcatggccttataaggtaggaccaggacttcaggactctgtgagcttgaagtccaggggtcccatcctggctatcctctcctcatgattggtgggtctgcaggaagatagcttttttaacctgcccatccaattcagtcaccctaaggttgagcgagtgatatgtctgcaaaaaagttgttaaaggttgggatacattcaacgtaagttctgtgtcagtcagtcccccctcccttagctccaacattgaaattgtccttgaccctcacgcacatgatagccagtgtgttgattgtcatttttgacgttgcaaatttcaagataggaaaaggctcagtgaccagtttaggatagccataacctatctcggtcattggcatctttggagtgacatccaaggatggaccatcccacctgtgtacaatgataatactttagctgtgtgtgtgtgtgtgtgtgtgtgtgttcaggccatgtgtcagtcaaccatttaatagatgagtgttaatttattatagataaatattgatctttaaaaatgggatgctcttaattcatgactttttaaagaattggattaaatatagctacttttatgtgagctttcccctctgtccccctgccccttctctctgtctcatttctcacaaccatcaccctccagtgtcacccccaaggctatatctccaacatactgctatgccattaaggttttatttaactttatttatggataattatgtctatgaacaacaaatgtgccgttaccggtcacacagtgtgtgggataagagtggcctagtgttgctttccgaatgtcttaaaatacctgtggtcaagattgtttttgcctttgtggcctttgggtttggagagcagcattgtgttcacatgtgtggatgtctgtcgtgctgctacagttgtaacactggagctggaaaaaataaagcattgatcttg